(19) United States
(12) Patent Application Publication
(10) Pub. No.: US 2008/0200454 A1
(43) Pub. Date: Aug. 21, 2008

(54) CARBON-LINKED TETRAHYDRO-PYRAZOLO-PYRIDINE MODULATORS OF CATHEPSIN S

(76) Inventors: Michael K. Ameriks, San Diego, CA (US); Frank U. Axe, Sutter Creek, CA (US); James P. Edwards, San Diego, CA (US); Cheryl A. Grice, Carlsbad, CA (US); Hui Cai, San Diego, CA (US); Elizabeth Ann Gleason, San Diego, CA (US); Steven P. Meduna, San Diego, CA (US); Kevin L. Tays, Cardiff by the Sea, CA (US); John J. M. Wiener, La Jolla, CA (US); Alvah T. Wickboldt, San Diego, CA (US)

Correspondence Address: WOODCOCK WASHBURN LLP, CIRA CENTRE, 12TH FLOOR, 2929 ARCH STREET, PHILADELPHIA, PA, 19104, US

(21) Appl. No.: 12/031,010

(22) Filed: Feb. 14, 2008

Related U.S. Application Data
(60) Provisional application No. 60/889,977, filed on Feb. 15, 2007.

(51) Int. Cl. A61K 31/437; A61P 37/00; A61P 29/00; A61P 1/00; A61P 25/00; A61P 35/00; A61K 31/496; A61K 31/541; A61K 31/55; A61P 17/00; A61P 3/10; A61P 11/00; C07D 471/04; A61K 31/53

(52) U.S. Cl. 514/217.07; 544/127; 514/234.2; 514/303; 546/119; 544/362; 514/253.04; 540/599; 544/58.6; 514/228.5

(57) ABSTRACT

Carbon-linked tetrahydro-pyrazolo-pyridine compounds are described, which are useful as cathepsin S modulators. Such compounds may be used in pharmaceutical compositions and methods for the treatment of disease states, disorders, and conditions mediated by cathepsin S activity, such as psoriasis, pain, multiple sclerosis, atherosclerosis, and rheumatoid arthritis.

This application claims the benefit of U.S. provisional patent application Ser. No. 60/889,977, filed Feb. 15, 2007 which is incorporated herein by reference.

### FIELD

The present invention relates to certain carbon-linked tetrahydro-pyrazolo-pyridine compounds, pharmaceutical compositions containing them, and methods of using them for the treatment of disease states, disorders, and conditions mediated by cathepsin S activity.

### BACKGROUND

Cathepsin S is one of the major cysteine proteases expressed in the lysosome of antigen presenting cells, mainly dendritic cells, B cells and macrophages. Cathepsin S is best known for its critical function in the proteolytic digestion of the invariant chain chaperone molecules, thus controlling antigen presentation to CD4⁺ T cells by major histocompatibility complex class II molecules or to NK1.1⁺ T cells via CD1 molecules. Cathepsin S also appears to participate in direct processing of exogenous antigens for presentation by MHC class II to CD4⁺ T cells or crosspresentation by MHC class I molecules to CD8⁺ T cells. In addition, cathepsin S in secreted form is implicated in degradation of extracellular matrix, which may contribute to the pathology of a number of diseases, including arthritis, atherosclerosis, and chronic obstructive pulmonary disease. Therefore, inhibition of cathepsin S is a promising target for the development of novel therapeutics for a variety of indications. For a review, see: Thurmond, R. L. et al. Curr. Opin. Invest. Drugs 2005, 6(5), 473-482.

Pyrazole inhibitors of cathepsin S were disclosed in a series of applications from Ortho-McNeil, and additional publications on this work have also appeared (See: U.S. Pat. Appl. Publ. Nos. 2002/0040020, 2003/0078419, and 2002/0040019; Wei, J. et al. Bioorg. Med. Chem. Lett. 2007, 17, 5525-5528; Grice, C. A. et al. Bioorg. Med. Chem. Lett. 2006, 16, 2209-2212; and Gustin, D. J. et al. Bioorg. Med. Chem. Lett. 2005, 15, 1678-1691). See also: Thurmond, R. L. et al. J. Pharm. Exp. Ther. 2004, 308, 268-276; and Thurmond, R. L. et al. J. Med. Chem. 2004, 47, 4799-4801). However, there remains a need for potent cathepsin S modulators with desirable pharmaceutical properties.

### SUMMARY

In one aspect the invention relates to compounds of the following Formula (I):

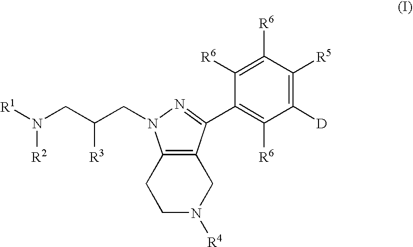

wherein:
R¹ and R² taken together with the nitrogen to which they are attached form a saturated monocyclic heterocycloalkyl group, each group optionally containing one additional heteroatom ring member that is O, S, or NR^{a}, and each group unsubstituted or substituted with one, two, or three R^{b} substituents; where R^{a} is H, C₁₋₄alkyl, —COC₁₋₄alkyl, —CO(phenyl), or —CO₂C₁₋₄alkyl, or a monocyclic cycloalkyl ring, phenyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
   each R^{b} substituent is independently:
   i) OH, C₁₋₄alkyl, —C₁₋₄alkyl-OH, CF₃, —NR^{c}R^{d}, halo, —OC₁₋₄alkyl, —COC₁₋₄alkyl, —CO₂C₁₋₄alkyl, —CO₂H, or —CONR^{e}R^{f};
   ii) a monocyclic heterocycloalkyl group unsubstituted or substituted with C₁₋₄alkyl, —COC₁₋₄alkyl, —CO₂C₁₋₄alkyl, OH, —OC₁₋₄alkyl, —NR^{c}R^{d}, or halo;
   iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
   iv) a phenyl group or monocyclic heteroaryl group, each group unsubstituted or substituted with C₁₋₄alkyl, OH, —OC₁₋₄alkyl, —NR^{c}R^{d}, or halo; or
   v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo;
   vi) two R substituents form a methylene or ethylene bridge; or
   vii) two R substituents on adjacent carbons taken together with the carbons to which they are attached form a saturated monocyclic cycloalkyl group or saturated monocyclic heterocycloalkyl group, each group unsubstituted or substituted with C₁₋₄alkyl, OH, —OC₁₋₄alkyl, —NR^{c}R^{d}, or halo;
      where R^{c} is H or C₁₋₄alkyl;
      R⁴ is H, C₁₋₄alkyl, —COC₁₋₄alkyl, —COC₁₋₄alkyl-OH, —CO₂C₁₋₄alkyl, —CONR^{x}R^{y}, or —SO₂C₁₋₄alkyl;
      where R^{x} and R^{y} are each independently H or C₁₋₄alkyl; and
      R^{e} and R^{f} are each independently H or C₁₋₄alkyl;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, or —NR^{t}R^{u}; —COCF₃; —CO-(monocyclic heteroaryl); —CO—(C-linked monocyclic heterocycloalkyl); —CO-(phenyl); —SO₂C₁₋₄alkyl; —SO₂CF₃; —SO₂NR^{t}R^{u}; —CONR^{t}R^{u}; —COCO₂C₁₋₄alkyl; or —COCONR^{t}R^{u};
   where R^{t} and R^{u} are each independently H, C₁₋₄alkyl, or —COC₁₋₄alkyl; or R^{t} and R^{u} taken together with the nitrogen to which they are attached form a monocyclic heterocycloalkyl ring;
R⁵ is halo or CF₃;
each R⁶ is independently H or F;
D is —C≡C—R⁷, —CH═CH—R³, —(CH₂)₂₋₃—R³, or —(CH₂)₃₋₅—R⁹;
   where R⁷ is:
   I) a C₁₋₄alkyl group unsubstituted or substituted with OH, —OC₁₋₄alkyl, —NR^{g}R^{h}, phenyl, or phenoxy, each phenyl or phenoxy being unsubstituted or substituted with C₁₋₄alkyl, OH, —OC₁₋₄alkyl, halo, or CF₃;
      where R^{g} and R^{h} are each independently H, C₁₋₄alkyl, —COC₁₋₄alkyl, —COphenyl, —CO₂C₁₋₄alkyl, —SO₂C₁₋₄alkyl, or —SO₂-phenyl; or R^{g} and R^{h} taken together with the nitrogen to which they are attached form a monocyclic heterocycloalkyl group; or
   II) a monocyclic cycloalkyl group, phenyl group, or monocyclic heteroaryl group, each group unsubstituted or substituted with one or two R^{k} substituents;
   where R⁹ is a phenyl group or monocyclic heteroaryl group, each group unsubstituted or substituted with one or two R^{k} substituents;
   where R⁹ is OH or —NRⁿR^{o};
      where Rⁿ is H or C₁₋₄alkyl; and
      R^{o} is H, C₁₋₄alkyl, monocyclic cycloalkyl, —COC₁₋₄alkyl, —COphenyl, —CO₂C₁₋₄alkyl, —SO₂C₁₋₄alkyl, —SO₂-phenyl, —SO₂-benzyl, or —SO₂NR^{p}R^{g}, each phenyl or benzyl group being unsubstituted or substituted with one or two R^{k} substituents; where R^{p} and R^{g} are each independently H or C₁₋₄alkyl;
      or Rⁿ and R^{o} taken together with the nitrogen to which they are attached form a monocyclic saturated heterocycloalkyl ring unsubstituted or substituted with C₁₋₄alkyl, OH, —OC₁₋₄alkyl, halo, or CF₃;
   where each R^{k} substituent in D is independently:
   a) a C₁₋₄alkyl group unsubstituted or substituted with OH, —OC₁₋₄alkyl, —OC₁₋₄alkyl-OH, halo, —CO₂C₁₋₄alkyl, CO₂H, CN, —NR^{r}R^{s}, —N(R^{r})COphenyl, —N(R^{r})SO₂C₁₋₄alkyl, —N(R^{r}) SO²-phenyl, —SO₂C₁₋₄alkyl, phenyl, or phenoxy;
   b) a substituent of formula

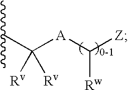

where each R^{v} is independently H or C₁₋₄alkyl, or both R^{v} substituents together form a carbonyl;
R^{w} is H, C₁₋₄alkyl, —CH₂OH, or —CO₂C₁₋₄alkyl;
A is O or NR^{aa};
   where R^{aa} is H or C₁₋₄alkyl; and
Z is a phenyl group, benzyl group, cycloalkyl group, heterocycloalkyl group, heteroaryl group, or —CH₂— (heteroaryl) group, each group unsubstituted or substituted with one or two substituents independently selected from the group consisting of C₁₋₄alkyl, CF₃, halo, OH, —OC₁₋₄alkyl, —OCF₃, —OCHF₂, NR^{dd}R^{ee}, —CO₂C₁₋₄alkyl, —SC₁₋₄alkyl, and —SO₂C₁₋₄alkyl;
   where R^{dd} and R^{ee} are each independently H or C₁₋₄alkyl;
c) two adjacent R^{k} substituents taken together with the carbons to which they are attached form a fused phenyl ring, monocyclic heteroaryl ring, monocyclic heterocycloalkyl ring, or monocyclic cycloalkyl ring, each fused ring unsubstituted or substituted with: C₁₋₄alkyl, —C₁₋₄alkyl-CF₃, —C₁₋₄alkyl-OH, —C₁₋₄alkyl-CO₂C₁₋₄alkyl, CF₃, C₂₋₄alkenyl, halo, OH, —OC₁₋₄alkyl, —COC₁₋₄alkyl, —COCF₃, —CO₂C₁₋₄alkyl, —CO₂H, —CONR^{ff}R^{gg}, or —SO₂C₁₋₄alkyl; or with a cycloalkyl group, —CH₂-(cycloalkyl) group, or benzyl group, each group unsubstituted or substituted with C₁₋₄alkyl, OH, —OC₁₋₄alkyl, halo, or CF₃;
   where R^{ff} and R^{gg} are each independently H or C₁₋₄alkyl, or R^{ff} and R^{gg} taken together with the nitrogen to which they are attached form a monocyclic heterocycloalkyl ring, unsubstituted or substituted with C₁₋₄alkyl or OH; or
d) OH; —OC₁₋₄alkyl; halo; CF₃; —CHO; —CO₂C₁₋₄alkyl; CO₂H; CN; —NO₂; —CONR^{r}R^{s}, —NR^{r}R^{s}; —N(R^{r})-phenyl; —N(R^{r})-benzyl; —N(R^{r})-phenethyl; —N(R^{r})COC₁₋₄-alkyl; —N(R^{r})CO-phenyl; —N(R^{r})SO₂C₁₋₄alkyl; —N (R^{r})SO₂-phenyl; —SO₂C₁₋₄alkyl; phenoxy; or a heteroaryl group; where each phenyl, benzyl, phenethyl, phenoxy, or heteroaryl group is unsubstituted or substituted with C₁₋₄alkyl, OH, —OC₁₋₄alkyl, halo, or CF₃;
   where R^{r} is H, C₁₋₄alkyl, C₂₋₄alkyl-OH; and
   R^{s} is H, C₁₋₄alkyl, —C₁₋₄alkyl-CF₃, —C₁₋₄alkyl-CN, —C₂₋₄alkyl-OH, —C₂₋₄alkyl-NR^{bb}R^{cc}, —C₁₋₄alkylCO₂C₁₋₄alkyl, —C₁₋₄alkylCO₂H, C₃₋₄alkenyl, —COC₁₋₄alkyl, or —CO₂C₁₋₄alkyl;
      where R^{bb} is H or C₁₋₄alkyl; and R^{cc} is H, C₁₋₄alkyl, —COC₁₋₄alkyl, or —CO₂C₁₋₄alkyl;
      or R^{bb} and R^{cc} taken together with the nitrogen to which they are attached from a monocyclic heterocycloalkyl ring;
   or R^{r} and R^{s} taken together with the nitrogen to which they are attached form a heterocycloalkyl group unsubstituted or substituted with C₁₋₄alkyl, OH, —OC₁₋₄alkyl, halo, CF₃, or a monocyclic heterocycloalkyl ring unsubstituted or
      substituted with OH;
         and pharmaceutically acceptable salts, prodrugs, and metabolites thereof.

In certain embodiments, the compound of Formula (I) is a compound selected from those species described or exemplified in the detailed description below.

In a further aspect, the invention relates to pharmaceutical compositions each comprising: (a) an effective amount of at least one chemical entity selected from compounds of Formula (I), and pharmaceutically acceptable salts, prodrugs, and metabolites thereof; and (b) a pharmaceutically acceptable excipient.

In another aspect, the invention is directed to a method of treating a subject suffering from or diagnosed with a disease, disorder, or medical condition mediated by cathepsin S activity, comprising administering to the subject in need of such treatment an effective amount of at least one chemical entity selected from compounds of Formula (I), and pharmaceutically acceptable salts, prodrugs, and metabolites thereof. Diseases, disorders and medical conditions that are mediated by cathepsin S activity include those referred to herein.

In another aspect, the chemical entities of the present invention are useful as cathepsin S modulators. Thus, the invention is directed to a method for modulating cathepsin S activity, including when such receptor is in a subject, comprising exposing cathepsin S to an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I).

Additional embodiments, features, and advantages of the invention will be apparent from the following detailed description and through practice of the invention.

### DETAILED DESCRIPTION

For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a saturated, straight- or branched-chain alkyl group having from 1 to 12 carbon atoms in the chain. Examples of alkyl groups include methyl (Me, which also may be structurally depicted by a bond, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “alkenyl” refers to a straight- or branched-chain alkenyl group having 2 to 12 carbons in the chain. Examples of alkenyl groups include vinyl, propenyl, 2-methyl-propenyl, butenyl, butadienyl, 2-methyl-butenyl, pentenyl, 2-methyl-pentenyl, 2-ethyl-pentenyl, 3-methyl-pentenyl, hexenyl, and groups that in light of the ordinary skill in the art and the teachings herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

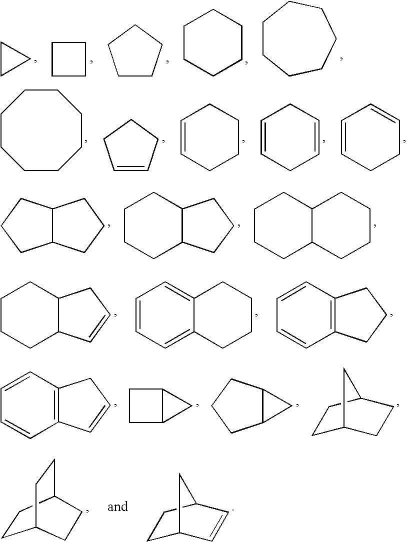

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. In preferred embodiments, heterocycloalkyl rings have one or two heteroatoms. The ring structure may optionally contain up to two oxo groups on carbon or sulfur ring members. Illustrative entities, in the form of properly bonded moieties, include:

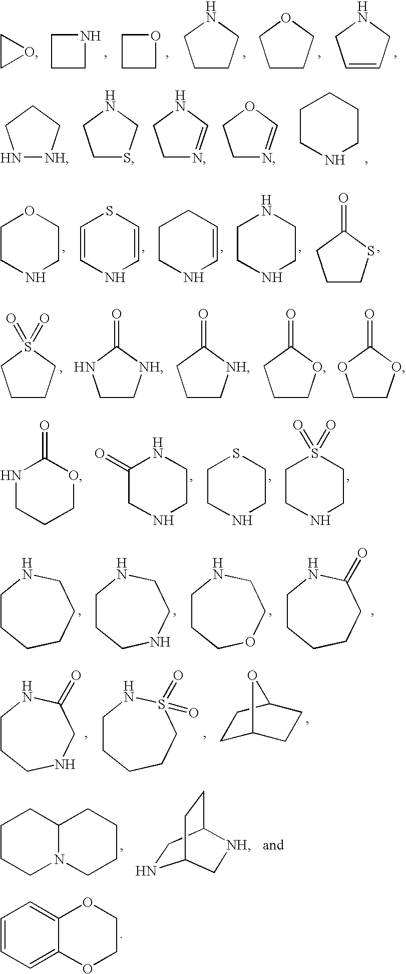

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

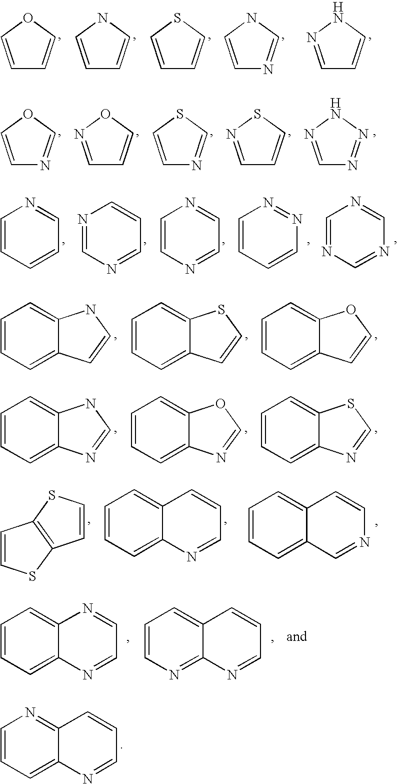

Those skilled in the art will recognize that the species of heteroaryl, cycloalkyl, and heterocycloalkyl groups listed or illustrated above are not exhaustive, and that additional species within the scope of these defined terms may also be selected.

The term “halogen” represents chlorine, fluorine, bromine, or iodine. The term “halo” represents chloro, fluoro, bromo, or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system that yields a stable chemical structure.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to represent hydrates, solvates, and polymorphs of such compounds, and mixtures thereof.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Reference to a chemical entity herein stands for a reference to any one of: (a) the actually recited form of such chemical entity, and (b) any of the forms of such chemical entity in the medium in which the compound is being considered when named. For example, reference herein to a compound such as R—COOH, encompasses reference to any one of, for example, R—COOH₍ₛ₎, R—COOH₍ₛₒₗ₎, and R—COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In this regard, if such entity is for example in an aqueous environment, it is understood that the compound R—COOH is in such same medium, and therefore the entity is being exposed to species such as R—COOH_{(aq)} and/or R—COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, salvation, including hydration, protonation, and deprotonation. No further examples in this regard are provided herein because these interactions and transformations in a given medium are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³³P, ³³P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this invention given according to the choices S¹ₑₓₐₘₚₗₑ is S¹ and S²ₑₓₐₘₚₗₑ is S₃; S¹ₑₓₐₘₚₗₑ is S¹ and S²ₑₓₐₘₚₗₑ is S₄; S example is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to any generic substituent symbol used herein.

Furthermore, when more than one assignment is given for any member or substituent, embodiments of this invention comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this invention for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to any generic substituent symbol used herein.

The nomenclature “Cᵢ₋ⱼ” with j>i, when applied herein to a class of substituents, is meant to refer to embodiments of this invention for which each and every one of the number of carbon members, from i to j including i and j, is independently realized. By way of example, the term C₁₋₃ refers independently to embodiments that have one carbon member (C₁), embodiments that have two carbon members (C₂), and embodiments that have three carbon members (C₃).

The term Cₙ₋ₘalkyl refers to an aliphatic chain, whether straight or branched, with a total number N of carbon members in the chain that satisfies n≦N≦m, with m>n.

Any disubstituent referred to herein is meant to encompass the various attachment possibilities when more than one of such possibilities are allowed. For example, reference to disubstituent -A-B-, where A≠B, refers herein to such disubstituent with A attached to a first substituted member and B attached to a second substituted member, and it also refers to such disubstituent with A attached to the second substituted member and B attached to the first substituted member.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

In some embodiments of Formula (I), —NR¹R² is a structure of Formula (II):

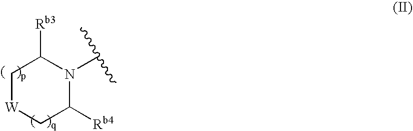

wherein:
W is NR^{a}, O, S, or C(R^{b1})(R^{b2});
   where R^{a} is H or C₁₋₄alkyl;
   R^{b1} is H, OH, or C₁₋₄alkyl; and
   R^{b2} is H; C₁₋₄alkyl; a monocyclic heterocycloalkyl group unsubstituted or substituted with C₁₋₄alkyl, OH, —OC₁₋₄alkyl, NR^{c}R^{d}, or halo; or a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
one of R^{b3} and R^{b4} is H and the other is H or C₁₋₄alkyl;
p is 0, 1, or 2; and
q is 0, 1, 2, or 3;
with the proviso that when W is NR^{a}, O, or S, then p and q are each greater than or equal to 1.

In other embodiments, —NR¹R² is a structure of Formula (III):

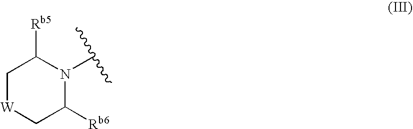

wherein W is O or S; and R^{b5} and R^{b6} are each independently H or C₁₋₄alkyl.

In other embodiments of Formula (I), R¹ and R² taken together with the nitrogen to which they are attached form azetidine, pyrrolidine, piperidine, piperazine substituted with R^{a}, morpholine, or thiomorpholine, each unsubstituted or substituted with one, two, or three R substituents as described for Formula (I). In other embodiments, R¹ and R² taken together with the nitrogen to which they are attached form piperidine, piperazine substituted with R^{a}, or morpholine, each unsubstituted or substituted with one, two, or three R substituents as described for Formula (I). In other embodiments, R¹ and R² taken together with the nitrogen to which they are attached form 1,1-dioxo-1λ⁶-thiomorpholine, thiomorpholine 1-oxide, piperazinone substituted with R^{a}, [1,4]oxazepane, each unsubstituted or substituted with one, two, or three R^{b} substituents; or 2,5-diaza-bicyclo[2.2.1]heptane substituted with R^{a}, 2-oxa-5-aza-bicyclo[2.2.1]heptane, 2-oxa-6-aza-spiro[3.3]heptane, or hexahydro-furo[3,4-c]pyrrole, each of the latter four groups unsubstituted or substituted with one R^{b} substituent.

In some embodiments, R^{a} is H, methyl, isopropyl, acetyl, or tert-butoxycarbonyl. In other embodiments, R^{a} is phenyl, 2-hydroxyphenyl, 3-hydroxyphenyl, 4-hydroxyphenyl, benzoyl, pyridyl, 1-hydroxy-pyridyl, or cyclobutyl.

In some embodiments, each R^{b} substituent is independently OH, methyl, CF₃, methoxycarbonyl, dimethylamino, acetamido, tert-butoxycarbamoyl, fluoro, or methoxy. In other embodiments, each R^{b} substituent is independently carbamoyl, amino, ethoxycarbonyl, carboxy, hydroxymethyl, 2-hydroxyacetylamino, methanesulfonylamino, or tert-butyl; or two R substituents on the same carbon taken together with the carbon to which they are attached form a dioxolane ring. In other embodiments, R^{b} is pyrrolidinyl, 2-oxo-pyrrolidinyl, or piperidinyl, each optionally substituted. In other embodiments, R^{b} is 2-oxo-piperidinyl, morpholinyl, 1-tert-butoxycarbonyl-piperidin-4-yl, 1-methyl-piperidin-4-yl, or 1-acetyl-piperidin-4-yl. In still other embodiments, R^{b} is pyrrolidin-1-yl, 2-oxo-pyrrolidin-1-yl, or 5-dimethylamino-1-methyl-1,3-dihydro-imidazo[4,5-b]pyridin-2-onyl, or two R^{b} substituents on the same carbon taken together with the carbon to which they are attached form 2-oxo-pyrrolidin-3-yl. In still other embodiments, R^{b} is phenyl or pyridyl, each optionally substituted.

In some embodiments, R³ is H or OH.

In some embodiments, R⁴ is —SO₂CH₃, —CONH₂, or —COCONH₂. In other embodiments, R⁴ is dimethylaminooxalyl, acetyl, dimethylsulfamoyl, methylcarbamoyl, dimethylcarbamoyl, 2-aminoacetyl, 2-acetoxyacetyl, 2-acetylamino-acetyl, tetrahydrofuran-2-carbonyl, or morpholine-4-carbonyl. In other embodiments, R⁴ is —SO₂CH₃.

In some embodiments, R⁵ is chloro or CF₃. In other embodiments, R⁵ is chloro.

In some embodiments, each R⁶ is H.

In some embodiments, D is —C≡C—R⁷, and R⁷ is benzyl, phenethyl, phenpropyl, hydroxymethyl, 2-hydroxyethyl, 3-hydroxypropyl, butyl, phenoxymethyl, 2-methyl propyl, diethylaminomethyl, (1,1-dioxo-1λ⁶-thiomorpholin-4-yl)-methyl, benzamidomethyl, or (benzenesulfonamido)methyl. In other embodiments, R⁷ is cyclopentyl, cyclohexyl, phenyl, thiophenyl, or pyridyl, each unsubstituted or substituted with one or two R^{k} substituents. In other embodiments, R⁷ is phenyl, unsubstituted or substituted with two R^{k} substituents. In still other embodiments, R⁷ is 1H-indol-5-yl, 4-cyanomethyl-phenyl, 3-cyanomethyl-phenyl, 4-hydroxymethyl-phenyl, 3-hydroxymethyl-phenyl, 4-hydroxy-phenyl, 4-(3-carboxy-propyl)-phenyl, 4-(2-carboxy-ethyl)-phenyl, 4-(methoxycarbonyl)methyl-phenyl, 3-(methoxycarbonyl)methyl-phenyl, thiophen-2-yl, 3,4-dichloro-phenyl, 4-(4-iodo-phenoxy)-phenyl, 4-carboxymethyl-phenyl, 3-carboxymethyl-phenyl, 4-phenoxy-phenyl, 4-bromo-phenyl, 4-carboxy-phenyl, pyridin-4-yl, pyridin-3-yl, pyridin-2-yl, thiophen-3-yl, 2-methoxy-phenyl, 3-chlorophenyl, 2-chlorophenyl, 3-hydroxyphenyl, 4-chlorophenyl, 4-methylphenyl, 4-trifluoromethylphenyl, 4-fluorophenyl, 4-methoxyphenyl, 2,4-difluorophenyl, 2-trifluoromethylphenyl, 2-methylphenyl, 3-trifluoromethylphenyl, 4-amino-phenyl, phenyl, 4-(tert-butoxcarbamoyl)methyl-phenyl, benzyl, phenethyl, phenpropyl, hydroxymethyl, 2-hydroxyethyl, 3-hydroxypropyl, butyl, cyclohexyl, (diethylamino)methyl, (1,1-dioxo-1λ⁶-thiomorpholin-4-yl)methyl, 2-methylpropyl, phenoxymethyl, (benzamido)methyl, (benzenesulfonamido)methyl, 4-acetamido-phenyl, 4-aminomethyl-phenyl, 4-(methanesulfonamido)methyl-phenyl, 4-(benzenesulfonamido)methyl-phenyl, 4-(acetamido)methyl-phenyl, 4-(benzamido)methyl-phenyl, 4-(benzylamino)methyl-phenyl, 4-(4-methyl-benzylamino)methyl-phenyl, 4-(4-chloro-benzylamino)methyl-phenyl, 4-[benzyl(methyl)amino]methyl-phenyl, 4-pyrrolidin-1-ylmethyl-phenyl, 4-piperidin-1-ylmethyl-phenyl, or 1,2,3,4-tetrahydro-isoquinolin-1-yl.

In some embodiments, each R^{k} substituent in D is independently a methyl or ethyl group unsubstituted or substituted with OH, methoxy, fluoro, —CO₂CH₃, CO₂H, CN, amino, tert-butoxycarbamoyl, methylsulfonamido, acetamido, pyrrolidinyl, or piperidinyl.

In still other embodiments, each R^{k} substituent in D is a methyl group substituted with NR^{r}R^{s}. In other embodiments, each R^{k} substituent in D is methylaminomethyl, dimethylaminomethyl, diethylaminomethyl, isobutylaminomethyl, tert-butoxycarbonylamino-methyl, (2-hydroxyethyl)aminomethyl, (3-hydroxypropyl)aminomethyl, (methoxycarbonylmethyl-amino)-methyl, (carboxymethyl-amino)-methyl, (2,2,2-trifluoroethyl-amino)-methyl, allylamino-methyl, (2-hydroxy-2-methyl-propylamino)-methyl, ethylaminomethyl, propylaminomethyl, [bis-(2-hydroxy-ethyl)-amino]-methyl, 3-hydroxy-propoxymethyl, phenylsulfonylamino-methyl, or benzoylamino-methyl. In other embodiments, each R^{k} substituent in D is 3,4-dihydro-1H-isoquinolin-2-ylmethyl, 1,3-dihydro-isoindol-2-ylmethyl, 4-(2-oxo-pyrrolidin-1-yl)-piperidin-1-ylmethyl, or 4-(4-hydroxy-2-oxo-pyrrolidin-1-yl)-piperidin-1-ylmethyl, morpholin-4-ylmethyl.

In still other embodiments, each R^{k} substituent in D is independently OH, methoxy, chloro, bromo, fluoro, CF₃, CO₂H, CN, amino, dimethylamino, acetylamino, methylsulfonamido, or methylsulfonyl. In other embodiments, each R^{k} substituent in D is phenoxy, 4-iodo-phenoxy, benzylamino, cyanomethyl-amino, benzimidazol-2-yl, phenethyl-amino, 3-(tert-butoxycarbonyl-methyl-amino)-propylcarbamoyl, 3-methylamino-propylcarbamoyl, pyrrolidine-1-carbonyl, 3-hydroxy-pyrrolidine-1-carbonyl, piperazine-1-carbonyl, [1,4]diazepane-1-carbonyl, 3-hydroxy-propylcarbamoyl, or 2-morpholin-4-yl-ethylcarbamoyl.

In further embodiments, R^{k} is a substituent of formula

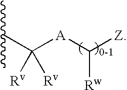

In other embodiments, R^{k} is phenethylamino-methyl, cyclopropylamino-methyl, cyclobutylamino-methyl, cyclopentylamino-methyl, cyclohexylamino-methyl, cyclopropylmethylamino-methyl, benzylamino-methyl, (4-chloro-benzylamino)-methyl, (4-methanesulfonyl-benzylamino)-methyl, (2-chloro-benzylamino)-methyl, (3-chloro-benzylamino)-methyl, (2-fluoro-benzylamino)-methyl, (3-fluoro-benzylamino)-methyl, (4-fluoro-benzylamino)-methyl, (3,4-dichloro-benzylamino)-methyl, (2-methoxy-benzylamino)-methyl, (3-methoxy-benzylamino)-methyl, (4-methoxy-benzylamino)-methyl, (2-methyl-benzylamino)-methyl, (3-methyl-benzylamino)-methyl, (4-methyl-benzylamino)-methyl, (4-dimethylamino-benzylamino)-methyl, (4-isopropoxy-benzylamino)-methyl, (4-difluoromethoxy-benzylamino)-methyl, (4-amino-benzylamino)-methyl, (benzyl)-methyl-amino)-methyl, [(4-chloro-benzyl)-methyl-amino]-methyl, (1-phenyl-ethylamino)-methyl, phenylaminomethyl, [(pyridin-2-ylmethyl)-amino]-methyl, [(pyridin-3-ylmethyl)-amino]-methyl, [(pyridin-4-ylmethyl)-amino]-methyl, (2-hydroxy-1-phenyl-ethylamino)-methyl, [(methoxycarbonyl-phenyl-methyl)-amino]-methyl, [(thiophen-2-ylmethyl)-amino]-methyl, [(thiophen-3-ylmethyl)-amino]-methyl, (2-thiophen-2-yl-ethylamino)-methyl, [(3-methyl-thiophen-2-ylmethyl)-amino]-methyl, [(furan-2-ylmethyl)-amino]-methyl, [(2-trifluoromethyl-furan-3-ylmethyl)-amino]-methyl, (1,2,3,4-tetrahydro-naphthalen-1-ylamino)-methyl, indan-1-ylaminomethyl, (2-hydroxy-indan-1-ylamino)-methyl, [(thiazol-2-ylmethyl)-amino]-methyl, [(1-methyl-1H-imidazol-2-ylmethyl)-amino]-methyl, [(tetrahydro-furan-2-ylmethyl)-amino]-methyl, or [(tetrahydro-pyran-4-ylmethyl)-amino]-methyl. In still other embodiments, R^{k} is (pyridin-2-ylmethyl)-carbamoyl, (pyridin-3-ylmethyl)-carbamoyl, (pyridin-4-ylmethyl)-carbamoyl, benzyl-carbamoyl, (4-chlorobenzyl)-carbamoyl, (pyrrolidin-2-ylmethyl)-carbamoyl, (pyrrolidin-3-ylmethyl)-carbamoyl, 2-hydroxy-1-phenyl-ethylcarbamoyl, (morpholin-2-ylmethyl)-carbamoyl, (piperidin-3-ylmethyl)-carbamoyl, or (azetidin-3-ylmethyl)-carbamoyl. In still other embodiments, R^{k} is pyridin-2-ylmethoxymethyl, pyridin-3-ylmethoxymethyl, pyridin-4-ylmethoxymethyl, piperidin-4-ylmethoxymethyl, morpholin-2-ylmethoxymethyl, pyrrolidin-3-yloxymethyl, or 1-tert-butoxycarbonyl-pyrrolidin-3-yloxymethyl.

In still further embodiments, two adjacent R^{k} substituents taken together with the ring to which they are attached form a bicyclic fused ring system selected from the group consisting of indole, tetrahydroisoquinoline, 3,4-dihydro-2H-isoquinolin-1-one, 2,3,4,5-tetrahydro-1H-benzo[d]azepine, 2,3,4,5-tetrahydro-1H-benzo[c]azepine, 2,3-dihydro-1H-isoindole, benzimidazole, imidazole, 1H-pyrrolo[2,3-b]pyridine, and 5,6,7,8-tetrahydro-[1,2,4]triazolo[4,3-a]pyrazine, each fused ring system optionally substituted. In other embodiments, the fused ring system is substituted with methyl, isopropyl, isobutyl, 2,2,2-trifluoroethyl, hydroxymethyl, ethoxycarbonylmethyl, allyl, acetyl, —COCF₃, tert-butoxycarbonyl, methoxycarbonyl, carboxy, carbamoyl, methylcarbamoyl, dimethylcarbamoyl, pyrrolidine-1-carbonyl, piperidine-1-carbonyl, 4-methyl-piperazine-1-carbonyl, or morpholine-4-carbonyl.

In some embodiments, R^{r} is H or methyl.

In some embodiments, R^{s} is H, methyl, acetyl, or tert-butoxycarbonyl. In still other embodiments, R^{r} and R⁵ taken together with the nitrogen to which they are attached form azetidinyl, pyrrolidinyl, or piperidinyl, each unsubstituted or substituted with methyl, OH, methoxy, fluoro, or CF₃.

In some embodiments, D is —CH═CH—R⁸ or —(CH₂)₂₋₃—R⁸, and R⁸ is phenyl, unsubstituted or substituted with one or two R^{k} substituents. In other embodiments, R⁸ is 1H-indol-5-yl, phenyl, 4-phenoxyphenyl, 3-hydroxyphenyl, 4-chlorophenyl, 4-methoxyphenyl, 2,4-difluorophenyl, 2-methylphenyl, or 4-hydroxymethyl-phenyl.

In some embodiments, D is —(CH₂)₃₋₅—R⁹, and R⁹ is OH. In other embodiments, R⁹ is NRⁿR^{o}. In other embodiments, R⁹ is dimethylamino, cyclopentylamino, acetamido, or methanesulfonamido. In other embodiments, R⁹ is benzamido, benzenesulfonamido, or benzylsulfonamido, each unsubstituted or substituted with one or two R^{k} substituents. In other embodiments, R⁹ is pyrrolidine, piperidine, morpholine, piperazine, or azepine, each unsubstituted or substituted with methyl, OH, fluoro, or CF₃. In other embodiments, R⁹ is OH, benzamido, methanesulfonamido, benzene-sulfonamido, benzylsulfonamido, 3,4-dichlorobenzenesulfonamido, 4-chlorobenzene-sulfonamido, 4-methylbenzenesulfonamido, 4-methoxybenzene-sulfonamido, N,N-dimethyl-sulfamoylurea, acetamido, 2-carboxybenzenesulfonamido, 2-nitrobenzene-sulfonamido, 3-chlorobenzenesulfonamido, 3-methoxybenzene-sulfonamido, 2-methylbenzene-sulfonamido, 2-chlorobenzenesulfonamido, 3-nitrobenzenesulfonamido, 3-methylbenzenesulfonamido, 3-cyanobenzenesulfonamido, 3-methanesulfonyl-benzenesulfonamido, 2-methanesulfonyl-benzenesulfonamido, pyrrolidin-1-yl, piperidin-1-yl, 3-methyl-pipieridin-1-yl, 4,4-difluoro-piperidin-1-yl, morpholin-4-yl, 4-methyl-piperazin-1-yl, azepan-1-yl, or cyclopentylamino.

In some embodiments, compounds of Formula (I) are selected from compounds of the following Formula (IV):

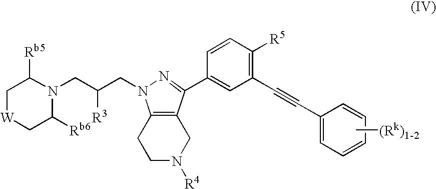

wherein:
W is O or S;
R^{b5} and R^{b6} are each independently H or C₁₋₄alkyl;
R³ is H or OH;
R⁴ is —SO₂CH₃, —CONH₂, or —COCONH₂;
R⁵ is chloro; and
each R^{k} substituent is independently:
a) a methyl group or ethyl group, each group unsubstituted or substituted with OH, —OC₁₋₄alkyl, halo, —CO₂C₁₋₄alkyl, CO₂H, CN, —NR^{r}R^{s}, —N(R^{r})COphenyl, —N(R^{r})SO₂C₁₋₄alkyl, —N(R^{r}) SO₂-phenyl, or —SO₂C₁₋₄alkyl;
   where R^{r} is H, C₁₋₄alkyl, C₂₋₄alkyl-OH; and
   R^{s} is H, C₁₋₄alkyl, C₁₋₄alkyl-CF₃, C₁₋₄alkyl-CN, C₂₋₄alkyl-OH, C₂₋₄alkyl-NR^{bb}R^{cc}, —C₁₋₄alkylCO₂C₁₋₄alkyl, —C₁₋₄alkylCO₂H, C₃₋₄alkenyl, —COC₁₋₄alkyl, or —CO₂C₁₋₄alkyl;
      where R^{bb} is H or C₁₋₄alkyl; and
      R^{cc} is H, C₁₋₄alkyl, —COC₁₋₄alkyl, or —CO₂C₁₋₄alkyl;
      or R^{bb} and R^{cc} taken together with the nitrogen to which they are attached from a monocyclic heterocycloalkyl ring;
   or R^{r} and R^{s} taken together with the nitrogen to which they are attached form a heterocycloalkyl group unsubstituted or substituted with C₁₋₄alkyl, OH, —OC₁₋₄alkyl, halo, CF₃, or a monocyclic heterocycloalkyl ring unsubstituted or substituted with OH;
b) a substituent of formula

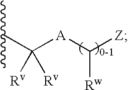

where each R^{v} is independently H or C₁₋₄alkyl, or both R^{v} substituents together form a carbonyl;
R^{w} is H, C₁₋₄alkyl, —CH₂OH, or —CO₂C₁₋₄alkyl;
A is O or NR^{aa};
   where R^{aa} is H or C₁₋₄alkyl; and
Z is a phenyl group, benzyl group, cycloalkyl group, heterocycloalkyl group, heteroaryl group, or —CH₂— (heteroaryl) group, each group unsubstituted or substituted with one or two substituents independently selected from the group consisting of C₁₋₄alkyl, CF₃, halo, OH, —OC₁₋₄alkyl, —OCF₃, —OCHF₂, NR^{dd}R^{ee}, —CO₂C₁₋₄alkyl, —SC₁₋₄alkyl, and —SO₂C₁₋₄alkyl;
   where R^{dd} and R^{ee} are each independently H or C₁₋₄alkyl; or
c) two adjacent R^{k} substituents taken together with the carbons to which they are attached form a fused phenyl ring, monocyclic heteroaryl ring, monocyclic heterocycloalkyl ring, or monocyclic cycloalkyl ring, each fused ring unsubstituted or substituted with: C₁₋₄alkyl, —C₁₋₄alkyl-CF₃, —C₁₋₄alkyl-OH, —C₁₋₄alkyl-CO₂C₁₋₄alkyl, CF₃, C₂₋₄alkenyl, halo, OH, —OC₁₋₄alkyl, —COC₁₋₄alkyl, —COCF₃, —CO₂C₁₋₄alkyl, —CO₂H, CONR^{ff}R^{gg}, or —SO₂C₁₋₄alkyl; or with a cycloalkyl group, —CH₂— (cycloalkyl) group, or benzyl group, each group unsubstituted or substituted with C₁₋₄alkyl, OH, —OC₁₋₄alkyl, halo, or CF₃;
   where R^{ff} and R^{gg} are each independently H or C₁₋₄alkyl, or R^{ff} and R^{gg} taken together with the nitrogen to which they are attached form a monocyclic heterocycloalkyl ring, unsubstituted or substituted with C₁₋₄alkyl or OH;
      and pharmaceutically acceptable salts, prodrugs, and metabolites thereof.

Other embodiments include combinations of the above variable definitions for Formula (I) and Formula (IV).

The invention includes also pharmaceutically acceptable salts of the compounds represented by Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), pharmaceutical compositions containing such pharmaceutically acceptable prodrugs, and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I), and uses of such metabolites in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites (collectively, “active agents”) of the present invention are useful in the methods of the invention.

The compounds of Formula (I) and their pharmaceutically acceptable salts, pharmaceutically acceptable prodrugs, and pharmaceutically active metabolites, whether alone or in combination, (collectively, “active agents”) of the present invention are useful as cathepsin S modulators in the methods of the invention. Such methods for modulating cathepsin S activity comprise exposing cathepsin S to an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I). Embodiments of this invention inhibit cathepsin S activity.

In some embodiments, the cathepsin S is in a subject with a disease, disorder, or medical condition mediated through modulation of the cathepsin S, such as those described herein. Symptoms or disease states are intended to be included within the scope of “medical conditions, disorders, or diseases.”

Accordingly, the invention relates to methods of using the active agents described herein to treat subjects diagnosed with or suffering from a disease, disorder, or condition mediated through cathepsin S activity, such as an autoimmune disease, an allergic condition, inflammation, a bowel disorder, tissue transplant rejection, pain, or cancer. Active agents according to the invention may therefore be used as immunomodulating agents, immunosuppressants, anti-allergy agents, anti-inflammatory agents, analgesics, or anti-cancer agents.

In some embodiments, an active agent of the present invention is administered to treat lupus, asthma, allergic reaction, atopic allergy, hay fever, atopic dermatitis, food allergy, rhinitis (such as allergic rhinitis and the inflammation caused by non-allergic rhinitis), skin immune system disorders (such as psoriasis), uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease (IBD), chronic obstructive pulmonary disorder (COPD), tissue transplant rejection, pain, neuropathic pain, chronic pain (such as pain due to conditions such as cancer, neuropathic pain, rheumatoid arthritis, osteoarthritis and inflammatory conditions), or cancer (and cancer-related processes such as angiogenesis, tumor growth, cell proliferation, and metastasis). In certain embodiments, an active agent of the present invention is administered to treat psoriasis, pain, multiple sclerosis, atherosclerosis, or rheumatoid arthritis.

Thus, the active agents may be used to treat subjects diagnosed with or suffering from a disease, disorder, or condition mediated through cathepsin S activity. The term “treat” or “treating” as used herein is intended to refer to administration of an active agent or composition of the invention to a subject for the purpose of effecting a therapeutic or prophylactic benefit through modulation of cathepsin S activity. Treating includes reversing, ameliorating, alleviating, inhibiting the progress of, lessening the severity of, or preventing a disease, disorder, or condition, or one or more symptoms of such disease, disorder or condition mediated through modulation of cathepsin S activity. The term “subject” refers to a mammalian patient in need of such treatment, such as a human. “Modulators” include both inhibitors and activators, where “inhibitors” refer to compounds that decrease, prevent, inactivate, desensitize or down-regulate cathepsin S expression or activity, and “activators” are compounds that increase, activate, facilitate, sensitize, or up-regulate cathepsin S expression or activity.

In treatment methods according to the invention, an effective amount of at least one active agent according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.001 to about 200 mg of active agent per kg of subject's body weight per day, preferably about 0.05 to 100 mg/kg/day, or about 1 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be coadministered separately with an active agent of Formula (I) or included with such an agent in a pharmaceutical composition according to the invention. In an exemplary embodiment, additional active ingredients are those that are known or discovered to be effective in the treatment of conditions, disorders, or diseases mediated by cathepsin S activity, such as another cathepsin S modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the active agent according to the invention.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary chemical entities useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, artisans will note that the various transformations described in the following Schemes may be performed in a different order than that depicted. Unless otherwise specified, the variables are as defined above in reference to Formula (I).

Abbreviations and acronyms used herein include the following:

| Term | Acronym |
|---|---|
| Tetrahydrofuran | THF |
| N,N-Dimethylformamide | DMF |
| N,N-Dimethylacetamide | DMA |
| Dimethyl sulfoxide | DMSO |
| Ethyl acetate | EtOAc |
| tert-Butylcarbamoyl | Boc |
| Bovine serum albumin | BSA |
| High-pressure liquid chromatography | HPLC |
| Thin layer chromatography | TLC |
| Diisobutylaluminum hydride | DIBAL-H |
| Acetate | OAc |
| Acetic acid | AcOH |
| O-(7-Azabenzotriazol-1-yl)-N,N.N′,N′- | HATU |
| tetramethyluronium hexafluorophosphate | |
| 1-Hydroxy-7-azabenzotriazole | HOAt |
| Diisopropylethylamine | DIPEA |
| 4-(Dimethylamino)pyridine | DMAP |
| 1-(3-Dimethylaminopropyl)-3- | EDC |
| ethylcarbodiimide hydrochloride | |
| 1-Hydroxybenzotriazole | HOBt |
| Methanesulfonyl chloride | MsCl |
| Tetrabutylammonium fluoride | TBAF |
| (Trimethylsilyl)acetylene | TMSA |
| Triethylamine | TEA |
| Trifluoroacetic acid | TFA |
| Phenyl | Ph |
| Dichloroethane | DCE |
| 1,8-Diazabicyclic[5.4.0]undec-7-ene | DBU |
| Methanol | MeOH |
| Ethanol | EtOH |
| Dibenzylideneacetone | dba |
| Diethyl ether | Et₂O |
| Isopropanol | IPA |

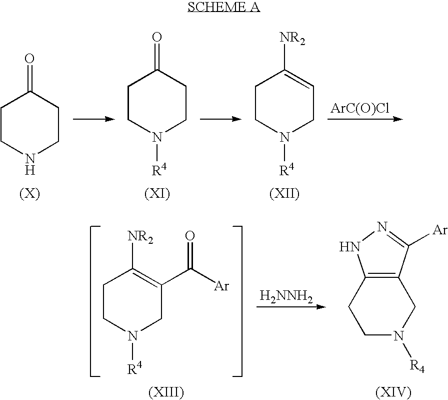

Referring to Scheme A, the tetrahydro-pyrazolo-pyridine core structure of Formula (I) may be prepared from commercially available piperidones (X). Installation of the R⁴ substituent is accomplished through, for example, alkylation, acylation, sulfonylation, amide formation, or other suitable methods known in the art to provide ketones (XI). Alternatively, an amine protecting group, such as a Boc group, may be installed, and, at a later point in the synthesis, be removed (for example, by treatment with an acid such as HCl or TFA) and replaced with R⁴ (See Scheme F). Enamine formation according to general methods gives enamines (XII), which are then reacted with acyl chlorides, ArC(O)Cl, where Ar is a suitable substituted phenyl group, in the presence of a suitable tertiary amine base, to form enamines (XIII) or the corresponding beta-diketones, or a mixture thereof (not isolated). In situ reaction of the enamines with hydrazine generates pyrazoles (XIV).

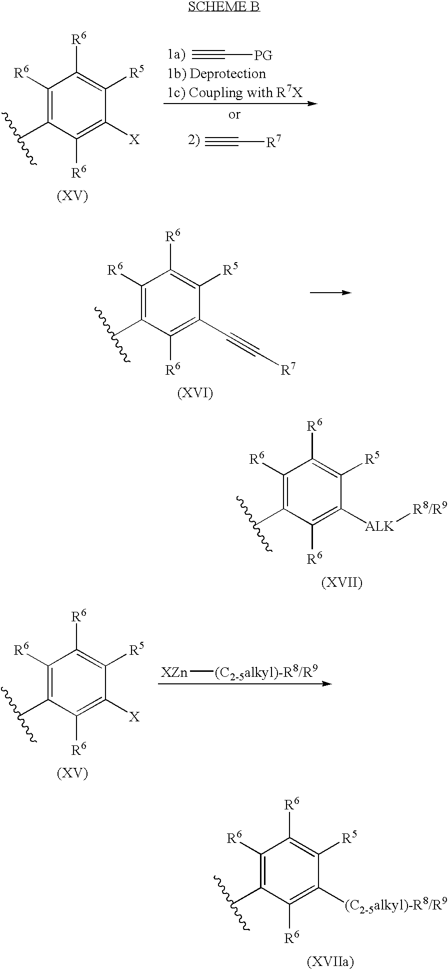

Where Ar is a suitably substituted group as in Formula (XV), where substituent X is iodide, bromide, or trifluoromethanesulfonate (triflate), formation of alkynes (XVI) may be accomplished by any one of several methods, as depicted in Scheme B. In some embodiments, a three-step process is employed, including: 1) palladium-catalyzed coupling with a suitably protected alkyne ≡-PG, where PG is, for example, a trialkylsilyl group such as trimethylsilyl; 2) deprotection of the alkyne with, for example, a fluoride source such as TBAF; and 3) coupling with a suitable reagent R⁷X, where R⁷ is as defined for Formula (I) and substituent X is as defined above, to give alkynes (XVI). Alternatively, palladium-mediated coupling of compounds (XV) with alkynes ≡-R⁷ yields alkynes (XVI) in one step. In preferred embodiments, palladium-catalyzed couplings are performed in the presence of a palladium(0) catalyst such as PdCl₂(PPh₃)₂, Pd(PPh₃)₄, or Pd₂(dba)₃, or a mixture thereof, a copper(1) salt such as copper(1) iodide, a tertiary amine base such as Et₃N or DBU, in a polar aprotic solvent such as THF or DMF or a mixture thereof, at a temperature from about room temperature to the reflux temperature of the solvent. Alkynes (XVI) may then be hydrogenated or otherwise reduced using methods known in the art to provide alkanes and alkenes (XVII), where ALK is C₂₋₅alkyl or C₂-alkenyl. In another embodiment, phenyl groups (XV) where substituent X is iodide may be coupled with suitably substituted Reformatsky reagents (where substituent X is preferably Cl) in the presence of a palladium catalyst to form alkanes (XVIIIa) in one step.

One skilled in the art will recognize that the chemistry described in Scheme B may be used with alkynes other than ≡-R⁷ or ≡-PG or reagents other than XZn(C₂₋₅alkyl)R⁸/R⁹ to generate other embodiments of Formula (I) or intermediates that are amenable to subsequent conversion into other embodiments of Formula (I). Examples of such conversions are shown in Scheme C. For example, where R⁷ is substituted with one or two R^{k} substituents, such substituents may be present for the couplings shown in Scheme B, or such substituents may be installed in subsequent reaction step(s).

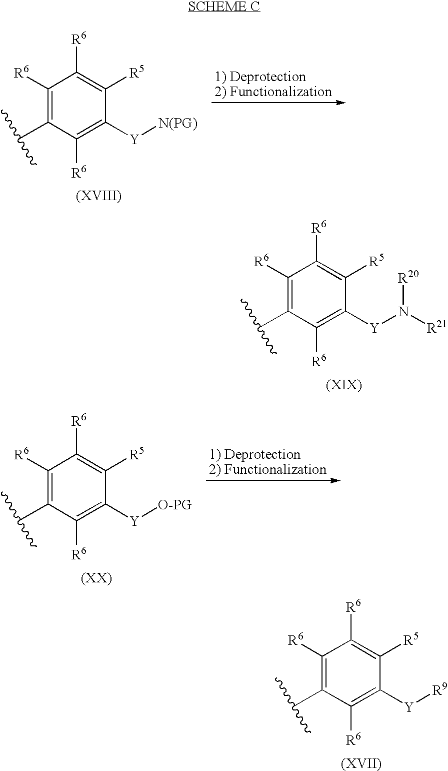

As shown in Scheme C, protected amines (XVIII), where Y is ethynyl, ethynyl-(C₁₋₄alkyl), or C₃₋₅alkyl, and PG is a suitable nitrogen protecting group, such as a Boc or phthalimide group, may be protected and converted into substituted amines (XIX). Exemplary transformations include reactions with suitably substituted alkyl halides, sulfonyl chlorides, acyl chlorides, or carboxylic acids. Alternatively, ethers (XX), where PG is H or a suitable hydroxyl protecting group, may be optionally deprotected and subsequently acylated, alkylated, or activated by conversion to the corresponding iodide, bromide, chloride, tosylate, or mesylate, and then displaced with suitable nucleophiles. The intermediate alcohols may also be oxidized to the corresponding aldehydes and reacted with amines using reductive amination methods known in the art.

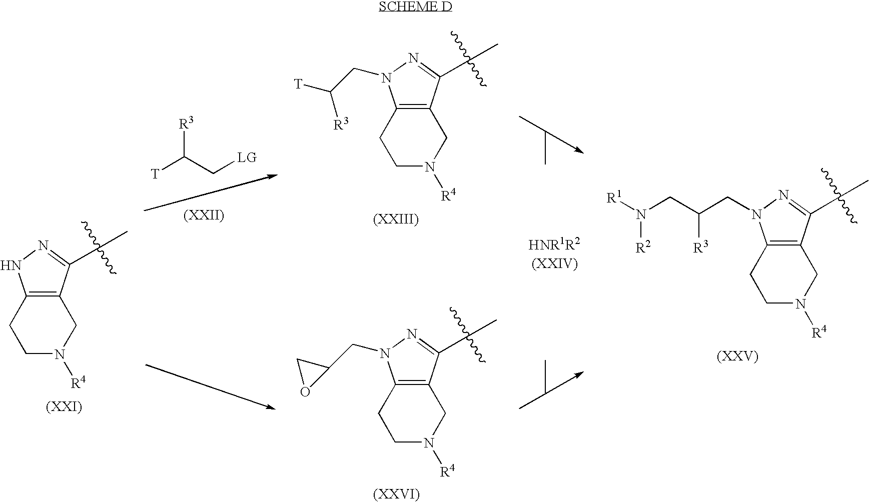

Two variations for the installation of the propyl amino chain are shown in Scheme D. One skilled in the art will recognize an amine protecting group, such as a Boc group, may be installed in place of the R⁴ shown, and, at a later point in the synthesis, be removed (for example, by treatment with an acid such as HCl or TFA) and replaced with R⁴. Pyrazoles (XXI) are alkylated with optionally protected reagents (XXII), where R³ is H, C₁₋₄alkyl, —OC₁₋₄alkyl, or a protected hydroxyl group, T is an aldehyde, a protected aldehyde, —CH₂—OH, a —CH₂— (protected hydroxyl) group, —CH₂—Cl, or —CH₂NR¹R², and LG is a suitable leaving group, such as a chloride, bromide, iodide, mesylate or tosylate, to give compounds (XXIII). Where T is a protected aldehyde (such as an acetal) or protected hydroxyl group, deprotection of (XXIII) is accomplished under general conditions. Resulting aldehydes are reacted with amines (XXIV) under reductive amination conditions, to provide propyl amines (XXV) where R³ is H, C₁₋₄alkyl, or —OC₁₋₄alkyl. Alcohols are converted to suitable leaving groups (LG), and displaced with amines HNR¹R², or are oxidized to the corresponding aldehydes for coupling with amines HNR¹R² by reductive amination. One skilled in the art will recognize that transformations from T to —CH₂NR¹R² may be performed at any one of several later points in the synthesis. Where T is —CH₂NR¹R², the alkylation step leads to compounds (XXV) directly.

Alternatively, pyrazoles (XXI) are reacted with epichlorohydrin or glycidyinosylate (each optionally as racemic mixtures or single enantiomers), in the presence of a suitable base, to give epoxides (XXVI). Epoxide opening with amines (XXIV), preferably at elevated temperatures, yields propyl amines (XXV) where R³ is OH.

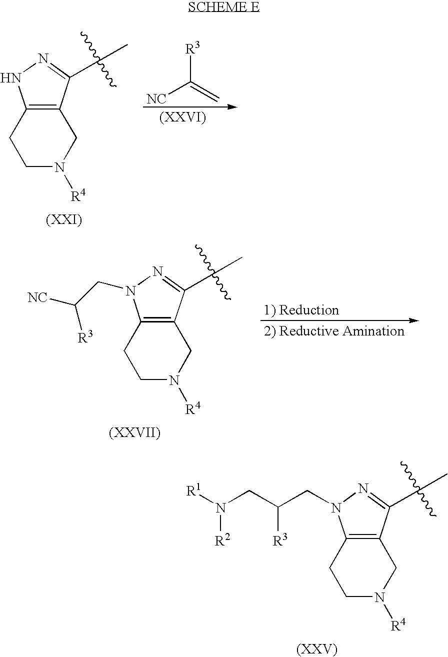

In another embodiment, shown in Scheme E, addition of pyrazoles (XXI) to α,β-unsaturated nitriles (XXVI), in the presence of a suitable base, such as aq. NaOH, generates nitriles (XXVII). Reduction of the nitriles to the corresponding aldehydes (XXIII, not shown) is accomplished with a reducing agent such as DIBAL-H. Reductive amination of aldehydes (XXIII) with amines (XXIV) gives amines (XXV) as described in Scheme D.

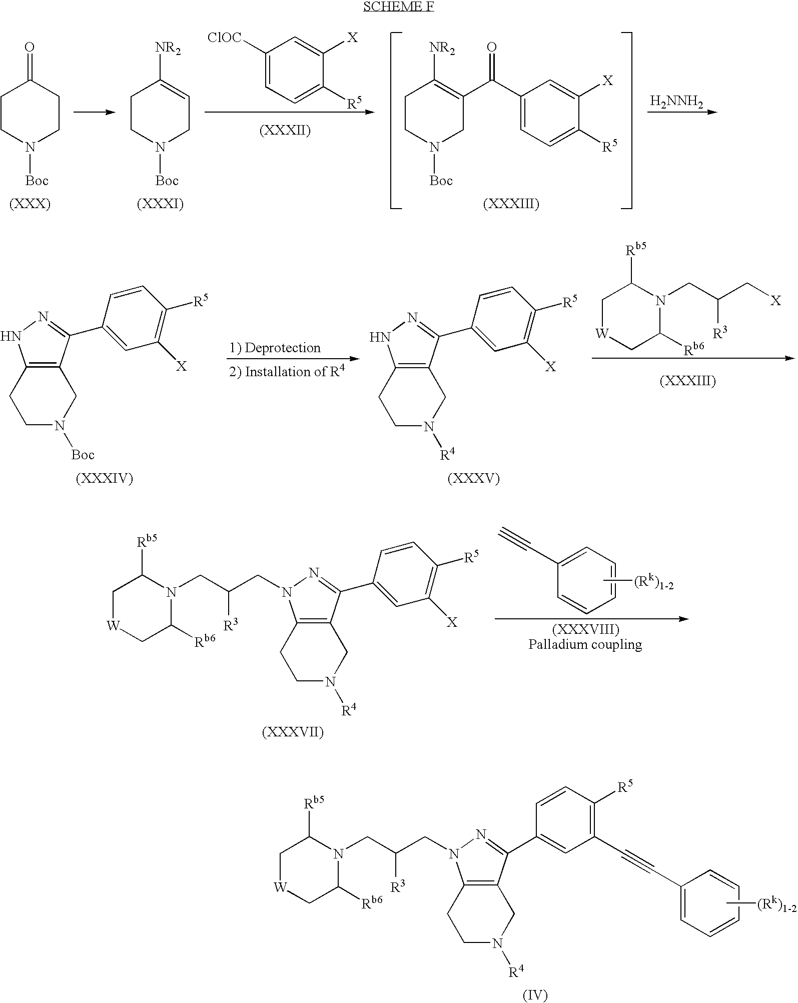

In preferred embodiments, compounds of Formula (IV) are prepared as shown in Scheme F. N-Boc-Piperidone (XXX) is reacted with cyclic secondary amines HNR₂, such as pyrrolidine, morpholine, or piperidine, in the presence of a catalytic amount of an acid catalyst such as p-toluenesulfonic acid or citric acid, in a solvent such as benzene or toluene, under dehydrating conditions (involving, for example, addition of molecular sieves or reaction at reflux temperature with a Dean-Stark trap), to form enamines (XXXI). Enamines (XXXI) are reacted with acid chlorides (XXXII, where substituent X is as defined above), prepared using methods known in the art, in the presence of a tertiary amine base such as TEA, DIPEA, or DBU, in a solvent such as CH₂Cl₂, DCE, or toluene, to provide enamines (XXXIII) or their corresponding beta-diketones (not shown), or a mixture thereof. Such compounds are not isolated, but are reacted directly with hydrazine, in a solvent such as MeOH or EtOH, to form pyrazoles (XXXIV).

The Boc protecting group of compounds (XXXIV) is removed using methods known in the art. In preferred embodiments, compounds (XXXIV) are treated with HCl or TFA in a solvent such as CH₂Cl₂ or 1,4-dioxane, to give the corresponding secondary amines (not shown, R⁴═H). Installation of R⁴ substituents other than H is accomplished using standard methods, including alkylation, acylation, amide coupling, sulfonylation, and other suitable transformations. In preferred embodiments, reaction with methanesulfonyl chloride in presence of a suitable tertiary amine base, or with oxamic acid in the presence of a coupling agent such as 1,1′-carbonyldiimidazole, provides pyrazoles (XXXV).

Alkylation of pyrazoles (XXXV) with reagents (XXXVI, where substituent X is iodo, bromo, chloro, mesylate or trifluoromethanesulfonate (triflate)), in the presence of a suitable base, gives compounds (XXXVII). Alkylations may also be performed as shown in Schemes D and E. In preferred embodiments, X in formula (XXXVI) is chloro, and reactions are performed using in DMF using Cs₂CO₃ as the base.

Compounds of Formula (IV) are prepared by reaction of compounds (XXXVII) under palladium-catalyzed conditions with alkynes (XXXVIII). In preferred embodiments, palladium-catalyzed couplings are performed in the presence of a palladium(0) catalyst such as PdCl₂(PPh₃)₂, Pd(PPh₃)₄, or Pd₂(dba)₃, or a mixture thereof, a copper(1) salt such as copper(1) iodide, a tertiary amine base such as TEA, DIPEA, or DBU, in a polar aprotic solvent such as THF or DMF or a mixture thereof, at a temperature from about room temperature to the reflux temperature of the solvent. In further preferred embodiments, substituents R^{k} are present in reagents (XXXVIII). In alternative embodiments, suitable surrogate substituents are present, and the R^{k} substituents are formed in subsequent reaction steps using standard chemical transformations.

Additional preferred embodiments, including preferred solvents, temperatures, reagents, and other conditions, are provided in the Examples below.

Compounds of Formula (I) may be converted to their corresponding salts using methods described in the art. For example, an amine of Formula (I) may be treated with trifluoroacetic acid, HCl, citric acid, oxalic acid, tartaric acid, 2-oxo-butanoic acid, 2-oxo-hexanoic acid, 2-keto-glutaric acid, 2-pyrrolidone-5-carboxylic acid, or phosphoric acid in a solvent such as CH₃CN, Et₂O, CH₂Cl₂, THF, or MeOH to provide the corresponding salt form. Alternatively, compounds of Formula (I) may be converted to their corresponding tartrate salts by reaction with tartaric acid in Et₂O, CH₂Cl₂, THF, or MeOH; to their corresponding monoethyl oxalate salts by reaction with mono- or diethyl oxalate in CH₃CN; or to their corresponding 2-oxo-pentanoate salts by reaction with 2-oxo-pentanoic acid in CH₃CN.

Those skilled in the art will recognize that the chemical transformations described above may be performed in a different order than that depicted in the above Schemes. Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following specific examples are provided to further illustrate the invention and various preferred embodiments.

### EXAMPLES

### Chemistry

In obtaining the compounds described in the examples below and the corresponding analytical data, the following experimental and analytical protocols were followed unless otherwise indicated.

Unless otherwise stated, reaction mixtures were magnetically stirred at room temperature (rt). Where solutions are “dried,” they are generally dried over a drying agent such as Na₂SO₄ or MgSO₄. Where mixtures, solutions, and extracts were “concentrated”, they were typically concentrated on a rotary evaporator under reduced pressure.

Microwave reactions were performed on a Personal Chemistry Emrys Optimizer. Individual reactions were heated to the desired temperature and held at that temperature for the allotted time.

Analytical HPLC retention times are reported in minutes, and were obtained on an Agilent HP-1100 instrument with a Phenomenex Luna C-18 (5 uM, 4.6×150 mm) column, with a flow rate of 1 mL/min, detection at 230, 254, and 280 nM, and a gradient of 10 to 100% CH₃CN (0.05% TFA)/H₂O (0.05% TFA).

Preparatory HPLC purifications were typically performed under one of the following sets of conditions:

Method A: Compounds were purified on a Phenomenex Synergi column (4 μm, 21×150 mm), with a flow rate of 25 mL/min, and solvent conditions as described for Analytical HPLC.
Method B: Compounds were injected onto a YMC column (C-18, 5 PM, 30×75 mm); with a flow rate of 30 mL/min; UV detection at 254 and 280 nM; and a gradient of 0 to 100% CH₃CN (0.05% TFA)/H₂O (0.05% TFA) over 21 min. The purified compounds were analyzed and tested as TFA salts following lyophilization, or as HCl salts following basic aqueous work up and treatment with dry HCl in 1,4-dioxane, Et₂O, or MeOH.
Method C: Compounds were injected onto an Intersil ODS-3 column (C-18, 3 μM, 30×100 mm); with a flow rate of 90 mL/min; UV detection at 254 and 280 nM; and a gradient of 0 to 60% CH₃CN/H₂O (0.05% TFA) over 2 min. The purified compounds were analyzed and tested as TFA salts following lyophilization, or as HCl salts following basic aqueous work up and treatment with dry HCl in 1,4-dioxane, Et₂O, or MeOH.
Method D: Compounds were injected onto an Intersil ODS-3 column (C-18, 3 μM, 30×100 mm); a flow rate of 90 mL/min; UV detection at 254 and 280 nM, and a gradient of 0 to 60% CH₃CN/H₂O (0.1% formic acid) over 2 min. The purified compounds were analyzed and tested as formic acid salts unless noted otherwise.
Method E: Compounds were injected onto a Phenomenex Luna column (C-18,10 μM, 50×250 mm); with a flow rate of 100 mL/min; UV detection at 254 and 280 nM; and a gradient of 0 to 100% CH₃CN (0.05% TFA)/H₂O (0.05% TFA) over 35 min. The purified compounds were analyzed and tested as TFA salts following lyophilization, or as HCl salts following basic aqueous work up and treatment with dry HCl in 1,4-dioxane, Et₂O, or MeOH.
Method F: Compounds were injected onto a Xbridge Prep column (C-18, 5 μM, 30×100 mm); with a flow rate of 30 mL/min; UV detection at 254 and 280 nM; and a gradient of 5 to 99% CH₃CN/H₂O (20 mM NH₄OH) over 18 min. The purified compounds were analyzed and tested as HCl salts following lyophilization and treatment with dry HCl in 1,4-dioxane, Et₂O, or MeOH.

Mass spectra (MS) were obtained on an Agilent series 1100 MSD using electrospray ionization (ESI) in positive mode unless otherwise indicated. The MS data presented is the m/z found (typically [M+H]⁺) for the molecular ion.

Nuclear magnetic resonance (NMR) spectra were obtained on Bruker model DRX spectrometers (400, 500, or 600 MHz). The format of the ¹H NMR data below is: chemical shift in ppm downfield of the tetramethylsilane reference (multiplicity, coupling constant J in Hz, integration). All ¹H NMR data was acquired in CD₃OD solvent unless otherwise indicated.

Chemical names were generated using ChemDraw Version 6.0.2 (CambridgeSoft, Cambridge, Mass.) or ACD/Name Version 9 (Advanced Chemistry Development, Toronto, Ontario, Canada).

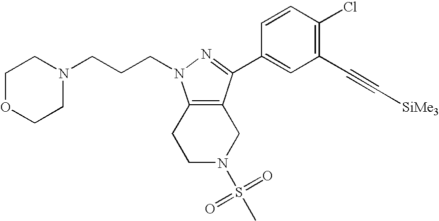

### Intermediate 1

### 3-(4-Chloro-3-trimethylsilanylethynyl-phenyl)-5-methanesulfonyl-1-(3-morpholin-4-yl-propyl)-4,5,6,7-tetrahydro-1H-pyrazolo[4,3-c]pyridine

A. 1-Methanesulfonyl-piperidin-4-one. To a solution of 4-piperidone monohydrate hydrochloride (90 g, 586 mmol) in CHCl₃ (300 mL) and H₂O (300 mL) was added K₂CO₃ (324 g, 2340 mmol). The slurry was cooled to 0 LC and treated with methanesulfonyl chloride (MsCl; 136 mL, 1.76 mol) by dropwise addition over a 1 h period (gas evolution was observed). The reaction mixture was allowed to stir for 72 h and was partitioned between CH₂Cl₂ (500 mL) and aq. NaHCO₃ (500 mL). The aqueous layer was extracted with CH₂Cl₂ (3×200 mL). The organic layer was washed with 1% KHSO₄ (250 mL), dried (Na₂SO₄), and concentrated to give the desired product (90.5 g, 87%) as a white solid. HPLC: Rₜ=2.2. MS (ESI): mass calcd. for C₆H₁₁NO₃S, 178.1; m/z found, 178.1 [M+H]⁺. ¹H NMR (CDCl₃): 3.60 (t, J=6.5, 4H), 2.89 (s, 3H), 2.59 (t, J=6.3, 4H).

B. 3-(4-Chloro-3-iodo-phenyl)-5-methanesulfonyl-4,5,6,7-tetrahydro-1H-pyrazolo[4,3-c]pyridine. To a solution of the above piperidone (10 g, 56 mmol) and p-toluenesulfonic acid (40 mg) in benzene (60 mL) was added morpholine (4.9 mL, 56 mmol). The reaction mixture was heated in a flask equipped with a condenser and a Dean-Stark trap at 90° C. for 16 h. The reaction mixture was cooled and concentrated to give the desired enamine as a beige solid, which was used without further purification. The enamine was dissolved in CH₂Cl₂ (40 mL), treated with TEA (9.4 mL, 67.2 mmol), and cooled to 0 LC. To this solution was added 4-chloro-3-iodobenzoyl chloride* (16.9 g, 56 mmol). The reaction mixture was allowed to warm to rt, stirred for 14 h, and then concentrated. The resulting red oil was diluted with EtOH (56 mL) and treated with hydrazine (5.34 mL, 170 mmol) at 0 LC. The resulting slurry was allowed to warm to rt and stirred for 16 h. EtOAc (120 mL) was added, and after 2 h the resulting precipitate was filtered and washed with additional EtOAc to afford the desired product as a white solid (8.80 g, 36%). HPLC: Rₜ=6.08. MS (ESI): mass calcd. for C₁₃H₁₃CIIN₃O₂S, 437.7; m/z found, 438.1 [M+H]⁺. ¹H NMR (DMSO-d₆): 8.05 (d, J=1.9, 1H), 7.51 (d, J=8.3, 1H), 7.43 (dd, J=8.4, 1.9, 2H), 4.30 (s, 2H), 3.36 (t, J=5.8, 2H), 3.30 (br s, 1H), 2.86 (s, 3H), 2.69 (t, J=5.6, 2H).

*4-Chloro-3-iodobenzoyl chloride was prepared by dissolving 4-chloro-3-iodobenzoic acid (15.8 g, 56 mmol) in CH₂Cl₂ (40 mL) and treating with oxalyl chloride (4.1 mL, 46.7 mmol) and a catalytic amount of DMF (400 μL; vigorous gas evolution). The mixture was stirred at rt for 3 h. The reaction mixture was concentrated to afford a white solid, which was used without further purification.

C. 3-(4-Chloro-3-iodo-phenyl)-1-(2-[1,3]dioxolan-2-yl-ethyl)-5-methanesulfonyl-4,5,6,7-tetrahydro-1H-pyrazolo[4,3-c]pyridine. A slurry of the above pyrazole (10 g, 22.8 mmol) and Cs₂CO₃ (11.9 g, 45.6 mmol) in DMF (75 mL) was stirred at rt for 2 h. 2-(2-Bromoethyl)1,3-dioxolane (3.5 mL, 34.2 mmol) was added dropwise and stirring maintained for 2 days. Ice water was added slowly to form a precipitate. The white solid was collected by suction filtration and washed with H₂O and Et₂O to afford the desired product (10.4 g, 85%). HPLC: Rₜ=6.98. MS (ESI): mass calcd. for C₁₈H₂₁CIIN₃O₄S, 537.8; m/z found, 538.2 [M+H]⁺. ¹H NMR (CDCl₃): 8.15 (s, 1H), 7.46-7.45 (m, 2H), 4.83 (t, J=4.6, 1H), 4.49 (s, 2H), 4.17 (t, J=7.1, 2H), 4.01-3.97 (m, 2H), 3.89-3.86 (m, 2H), 3.65 (t, J=5.8, 2H), 2.89 (s, 3H), 2.87 (t, J=5.8, 2H), 2.28-2.26 (m, 2H).

D. 3-(4-Chloro-3-iodo-phenyl)-5-methanesulfonyl-1-(3-morpholin-4-yl-propyl)-4,5,6,7-tetrahydro-1H-pyrazolo[4,3-c]pyridine. A mixture of the above acetal (4.65 g, 8.64 mmol) and 1 N HCl (19 mL) in acetone (75 mL) was heated at 55 LC for 5 h. The clear solution was concentrated to remove acetone, and the aqueous layer was extracted with CH₂Cl₂ (3×). The combined organic extracts were dried (Na₂SO₄), filtered, and concentrated to give a white solid, which was used directly in the next reaction. The crude aldehyde was dissolved in CH₂Cl₂ (80 mL) and morpholine (2.5 mL, 28.6 mmol) and acetic acid (1.0 mL) were added sequentially. After 10 min, NaB(OAc)₃H (3.48 g, 13 mmol) was added and stirring was continued for 2.5 days. After the addition of 1 N NaOH, the layers were separated and the aqueous layer was extracted with CH₂Cl₂ (3×). The combined organic extracts were washed with brine, dried (Na₂SO₄), filtered, and concentrated to give an orange oil. Purification (SiO₂; 0-3% 2 M NH₃ in MeOH/CH₂Cl₂) afforded the title compound as a white solid (2.9 g, 60%). HPLC: Rₜ=4.78. MS (ESI): mass calcd. for C₂₀H₂₆CIIN₄O₃S, 564.9; m/z found, 566.2 [M+H]⁺. ¹H NMR (CDCl₃): 8.15 (s, 1H), 7.46-7.45 (m, 2H), 4.49 (s, 2H), 4.09 (t, J=6.8, 2H), 3.70 (t, J=4.6, 4H), 3.64 (t, J=5.8, 2H), 2.90 (s, 3H), 2.89 (t, J=5.8, 2H), 2.44-2.39 (br m, 4H), 2.32 (t, J=6.8, 2H), 2.11-2.03 (m, 2H).

E. 3-(4-Chloro-3-trimethylsilanylethynyl-phenyl)-5-methanesulfonyl-1-(3-morpholin-4-yl-propyl)-4,5,6,7-tetrahydro-1H-pyrazolo[4,3-c]pyridine. To a solution of the above iodoarene (1.0 g, 1.8 mmol), PdCl₂(PPh)₃ (124 mg, 0.177 mmol), and CuI (34 mg, 0.177 mmol) in degassed THF (10 mL) was added TEA (1.2 mL, 8.85 mmol), followed by (trimethylsilyl)acetylene (TMSA; 276 μL). The reaction mixture was stirred under N₂ at rt for 17 h. Satd. aq. NaHCO₃ was added, and the aqueous layer was extracted with CH₂Cl₂ (3×). The combined organic extracts were washed with brine, dried (Na₂SO₄), filtered, and concentrated to give a brown oil. Purification (SiO₂; 0-3% 2 M NH₃ in MeOH/CH₂Cl₂) afforded the desired product as a beige solid (860 mg, 91%). HPLC: Rₜ=5.45. MS (ESI): mass calcd. for C₂₅H₃₅ClN₄O₃SSi, 535.2; m/z found, 536.3 [M+H]⁺. ¹H NMR (CDCl₃): 7.73 (d, J=2.0, 1H), 7.46 (dd, J=8.4, 2.1, 1H), 7.40 (d, J=8.2, 1H), 4.51 (s, 2H), 4.09 (t, J=6.8, 2H), 3.70 (t, J=4.6, 4H), 3.65 (t, J=5.8, 2H), 2.90 (s, 3H), 2.89 (t, J=5.7, 2H), 2.43-2.38 (br m, 4H), 2.32 (t, J=6.8, 2H), 2.10-2.03 (m, 2H), 0.29 (s, 9H).

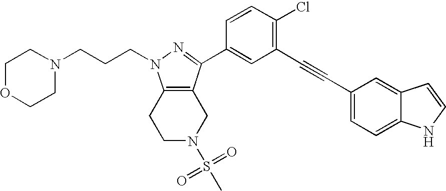

### Example 1

### 3-[4-Chloro-3-(1H-indol-5-ylethynyl)-phenyl]-5-methanesulfonyl-1-(3-morpholin-4-yl-propyl)-4,5,6,7-tetrahydro-1H-pyrazolo[4,3-c]pyridine

A. 3-(4-Chloro-3-ethynyl-phenyl)-5-methanesulfonyl-1-(3-morpholin-4-yl-propyl)-4,5,6,7-tetrahydro-1H-pyrazolo[4,3-c]pyridine. To a solution of 3-(4-chloro-3-trimethylsilanylethynyl-phenyl)-5-methanesulfonyl-1-(3-morpholin-4-yl-propyl)-4,5,6,7-tetrahydro-1H-pyrazolo[4,3-c]pyridine (947 mg, 1.77 mmol) in THF (7 mL) was added tetrabutylammonium fluoride (1.0 M in THF; 2.0 mL, 1.95 mmol) dropwise. The resulting solution was stirred at rt for 30 min, then diluted with H₂O. The aqueous layer was extracted with CH₂Cl₂ (3×). The combined organic extracts were washed with brine, dried (Na₂SO₄), filtered, and concentrated to give a black oil. Purification (SiO₂; 0-3% 2 M NH₃ in MeOH/CH₂Cl₂) provided the title compound as a yellow oil (550 mg, 67%). HPLC: Rₜ=4.62. MS (ESI): mass calcd. for C₂₂H₂₇ClN₄O₃S, 463.0; m/z found, 464.3 [M+H]⁺. ¹H NMR (CDCl₃): 7.75 (d, J=2.1, 1H), 7.52 (dd, J=8.4, 2.2, 1H), 7.43 (d, J=8.4, 1H), 4.49 (s, 2H), 4.09 (t, J=6.9, 2H), 3.71 (t, J=4.6, 4H), 3.64 (t, J=5.7, 2H), 2.91 (s, 3H), 2.88 (t, J=5.7, 2H), 2.47-2.42 (br m, 4H), 2.36 (t, J=7.1, 2H), 2.11-2.00 (m, 3H).

B. 3-[4-Chloro-3-(1H-indol-5-ylethynyl)-phenyl]-5-methanesulfonyl-1-(3-morpholin-4-yl-propyl)-4,5,6,7-tetrahydro-1H-pyrazolo[4,3-c]oyridine. To a solution of the above alkyne (120 mg, 0.259 mmol), 5-iodoindole (82 mg, 0.337 mmol), PdCl₂(PPh₃)₂ (18 mg, 0.026 mmol), and CuI (4.9 mg, 0.026 mmol) in degassed THF (2.4 mL) was added TEA (108 μL, 0.777 mmol). The reaction mixture was stirred at rt under N₂ for 16 h. The mixture was concentrated and the resulting black oil was purified (SiO₂; 0-5% 2 M NH₃ in MeOH/CH₂Cl₂) to provide the title compound as a white solid (132 mg, 88%). HPLC: Rₜ=4.73. MS (ESI): mass calcd. for C₃₀H₃₂ClN₅O₃S, 578.1; m/z found, 579.3 [M+H]⁺. ¹H NMR (CDCl₃): 8.60 (s, 1H), 7.92-7.90 (m, 1H), 7.80 (t, J=1.2, 1H), 7.44 (d, J=1.2, 2H), 7.40 (dd, J=8.4, 1.5, 1H), 7.35 (d, J=8.4, 1H), 7.23-7.20 (m, 1H), 6.56-6.54 (m, 1H), 4.50 (s, 2H), 4.07 (t, J=6.8, 2H), 3.69 (t, J=4.6, 4H), 3.60 (t, J=5.7, 2H), 2.88 (s, 3H), 2.83 (t, J=5.7, 2H), 2.41-2.37 (br m, 4H), 2.31 (t, J=6.8, 2H), 2.08-2.01 (m, 2H).

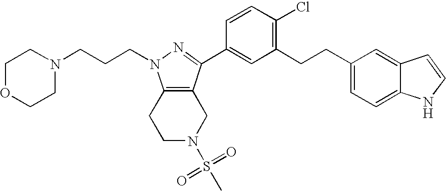

### Example 2

### 3-{4-Chloro-3-[2-(1H-indol-5-yl)-ethyl]-phenyl}-5-methanesulfonyl-1-(3-morpholin-4-yl-propyl)-4,5,6,7-tetrahydro-1H-pyrazolo[4,3-c]pyridine

A pressure tube containing a suspension of 3-[4-chloro-3-(1H-indol-5-ylethynyl)-phenyl]-5-methanesulfonyl-1-(3-morpholin-4-yl-propyl)-4,5,6,7-tetrahydro-1H-pyrazolo[4,3-c]pyridine (45 mg, 0.079 mmol) and PtO₂ (8.5 mg) in 1:1 EtOH/EtOAc (4 mL) was placed in a shaker under H₂ (20 psi). After 17 h, the reaction mixture was filtered through diatomaceous earth and concentrated to give a brown oil. Purification (SiO₂; 0-3% 2 M NH₃ in MeOH/CH₂Cl₂) provided the title compound as a white powder (39 mg, 85%). HPLC: Rₜ=5.10. MS (ESI): mass calcd. for C₃₀H₃₆ClN₅O₃S, 582.2; m/z found, 583.5 [M+H]⁺. ¹H NMR (CDCl₃): 8.25 (br s, 1H), 7.52-7.45 (m, 2H), 7.40 (d, J=8.2, 1H), 7.35-7.31 (m, 2H), 7.19-7.16 (m, 1H), 7.07 (dd, J=8.3, 1.5, 1H), 6.51-6.48 (m, 1H), 4.39 (s, 2H), 4.08 (t, J=6.8, 2H), 3.69 (t, J=4.6, 4H), 3.62 (t, J=5.8, 2H), 3.13-3.08 (m, 2H), 3.06-3.00 (m, 2H), 2.91-2.84 (m, 2H), 2.84 (s, 3H), 2.44-2.38 (br m, 4H), 2.32 (t, J=6.8, 2H), 2.11-2.02 (m, 2H).

The compounds in Examples 3-14 were prepared according to the methods described for Intermediate 1 and Example 1, substituting the appropriate iodide for 5-iodoindole in Example 1, Step B.

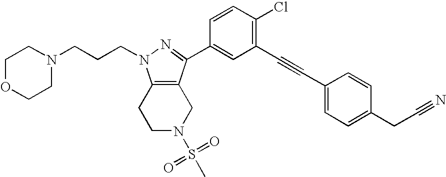

### Example 3

### (4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile

HPLC: Rₜ=4.81. MS (ESI): mass calcd. for C₃₀H₃₂ClN₅O₃S, 578.1; m/z found, 579.4 [M+H]⁺. ¹H NMR (CDCl₃): 7.81 (d, J=1.9, 1H), 7.62-7.59 (m, 2H), 7.50 (dd, J=8.4, 2.1, 2H), 7.34 (d, J=8.4, 2H), 4.54 (s, 2H), 4.10 (t, J=6.8, 2H), 3.79 (s, 2H), 3.70 (t, J=4.6, 4H), 3.65 (t, J=5.8, 2H), 2.91 (s, 3H), 2.90-2.87 (m, 2H), 2.43-2.38 (br m, 4H), 2.32 (t, J=6.8, 2H).

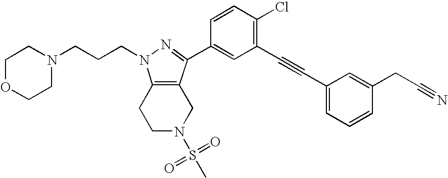

### Example 4

### (3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile

HPLC: Rₜ=5.43. MS (ESI): mass calcd. for C₃₀H₃₂ClN₅O₃S, 578.1; m/z found, 579.3 [M+H]⁺. ¹H NMR (CDCl₃): 7.80 (d, J=2.0, 1H), 7.59-7.55 (m, 2H), 7.53-7.33 (m, 4H), 4.55 (s, 2H), 4.11 (t, J=6.8, 2H), 3.78 (s, 2H), 3.70 (t, J=4.6, 4H), 3.66 (t, J=5.8, 2H), 2.91 (s, 3H), 2.90 (t, J=5.6, 2H), 2.43-2.39 (br m, 4H), 2.33 (t, J=6.8, 2H), 2.12-2.04 (m, 2H).

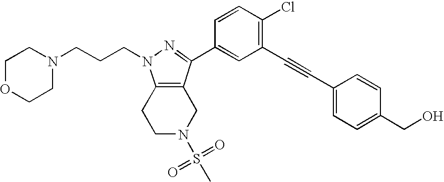

### Example 5

### (4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol

HPLC: Rₜ=4.82. MS (ESI): mass calcd. for C₂₉H₃₃ClN₄O₄S, 569.1; m/z found, 570.3 [M+H]⁺. ¹H NMR (CDCl₃): 7.80 (d, J=1.8, 1H), 7.61-7.57 (m, 2H), 7.51-7.44 (m, 2H), 7.37 (d, J=8.3, 2H), 4.73 (s, 2H), 4.54 (s, 2H), 4.10 (t, J=6.8, 2H), 3.69 (t, J=4.6, 4H), 3.65 (t, J=5.8, 2H), 2.90 (s, 3H), 2.90-2.87 (m, 2H), 2.43-2.38 (m, 4H), 2.32 (t, J=6.8, 2H), 2.11-2.04 (m, 2H), 1.87 (br s, 1H).

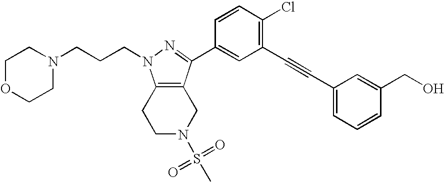

### Example 6

### (3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol

MS (ESI): mass calcd. for C₂₉H₃₃ClN₄O₄S, 569.1; m/z found, 570.3 [M+H]⁺.

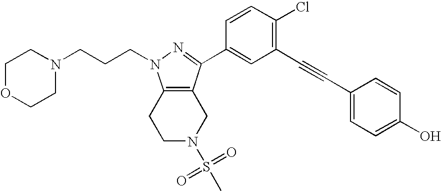

### Example 7

### 4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol

HPLC: Rₜ=4.61. MS (ESI): mass calcd. for C₂₈H₃₁ClN₄O₄S, 555.1; m/z found, 556.4 [M+H]⁺. ¹H NMR (CDCl₃): 7.77-7.75 (m, 1H), 7.42-7.37 (m, 4H), 6.81-6.77 (m, 2H), 4.50 (s, 2H), 4.11 (t, J=6.8, 2H), 3.70 (t, J=4.5, 4H), 3.61 (t, J=5.3, 2H), 2.91 (s, 3H), 2.86 (t, J=5.6, 2H), 2.46-2.41 (m, 4H), 2.37 (t, J=6.9, 2H), 2.12-2.04 (m, 2H).

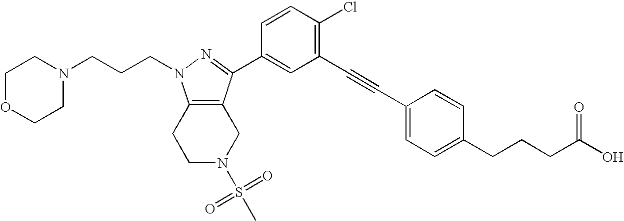

### Example 8

### 4-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-butyric acid

HPLC: Rₜ=4.89. MS (ESI): mass calcd. for C₃₂H₃₇ClN₄O₅S, 625.2; m/z found, 626.5 [M+H]⁺. ¹H NMR (CDCl₃): 7.77 (s, 1H), 7.52 (d, J=8.0, 2H), 7.47 (s, 2H), 7.21 (d, J=8.0, 2H), 4.52 (s, 2H), 4.11 (t, J=6.7, 2H), 3.74 (t, J=4.2, 4H), 3.68-3.63 (m, 4H), 3.10 (dd, J=14.6, 7.3, 1H), 2.93 (s, 3H), 2.92-2.88 (m, 2H), 2.69 (t, J=7.6, 2H), 2.53-2.47 (br m, 3H), 2.41 (t, J=7.0, 2H), 2.33 (t, J=7.4, 2H), 2.14-2.06 (m, 2H), 2.00-1.91 (m, 2H).

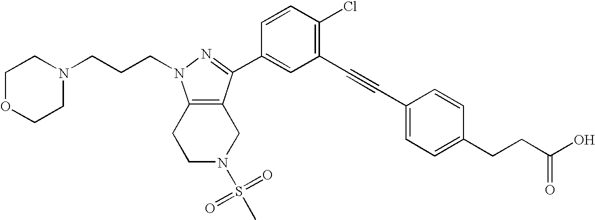

### Example 9

### 3-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-propionic acid

HPLC: Rₜ=4.64. MS (ESI): mass calcd. for C₃₁H₃₅ClN₄O₅S, 611.2; m/z found, 612.4 [M+H]⁺. ¹H NMR (CDCl₃): 7.70 (s, 1H), 7.45 (d, J=7.5, 2H), 7.38 (d, J=8.4, 2H), 7.22 (d, J=7.8, 2H), 6.68 (br s, 1H), 4.50 (s, 2H), 4.01 (t, J=6.5, 2H), 3.77-3.71 (m, 4H), 3.61-3.56 (m, 2H), 2.97 (t, J=7.1, 2H), 2.88 (s, 3H), 2.78-2.73 (m, 2H), 2.65 (t, J=7.1, 2H), 2.61-2.54 (m, 4H), 2.49 (t, J=6.7, 2H), 2.07 (t, J=6.6, 2H).

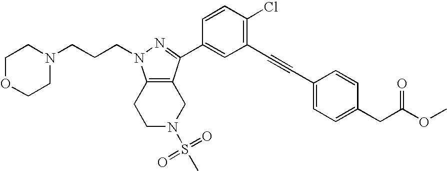

### Example 10

### (4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester

MS (ESI): mass calcd. for C₃₁H₃₅ClN₄O₅S, 611.2; m/z found, 612.3 [M+H]⁺.

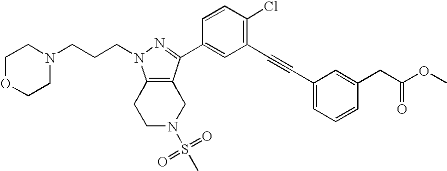

### Example 11

### (3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester

MS (ESI): mass calcd. for C₃₁H₃₅ClN₄O₅S, 611.2; m/z found, 612.3 [M+H]⁺.

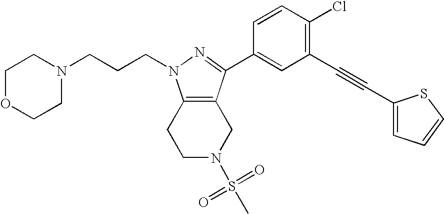

### Example 12

### 3-(4-Chloro-3-thiophen-2-ylethynyl-phenyl)-5-methanesulfonyl-1-(3-morpholin-4-yl-propyl)-4,5,6,7-tetrahydro-1H-pyrazolo[4,3-c]pyridine

HPLC: Rₜ=4.91. MS (ESI): mass calcd. for C₂₆H₂₉ClN₄O₃S₂, 545.1; m/z found, 546.3 [M+H]⁺. ¹H NMR (CDCl₃): 7.78 (d, J=2.0, 1H), 7.49 (dd, J=8.4, 2.0, 1H), 7.45 (d, J=8.4, 1H), 7.37-7.33 (m, 2H), 7.04 (dd, J=5.2, 3.7, 1H), 4.53 (s, 2H), 4.13-4.08 (m, 2H), 3.70 (t, J=4.6, 4H), 3.65 (t, J=5.8, 2H), 2.91 (s, 3H), 2.88 (t, J=5.8, 2H), 2.44-2.38 (br m, 4H), 2.33 (t, J=6.8, 2H), 2.11-2.03 (m, 2H).

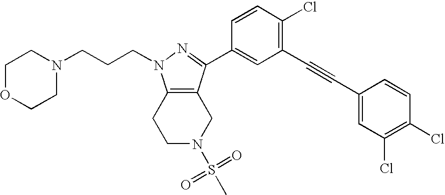

### Example 13

### 3-[4-Chloro-3-(3,4-dichloro-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₈H₂₉Cl₃N₄O₃S, 608.0; m/z found, 609.2 [M+H]⁺.

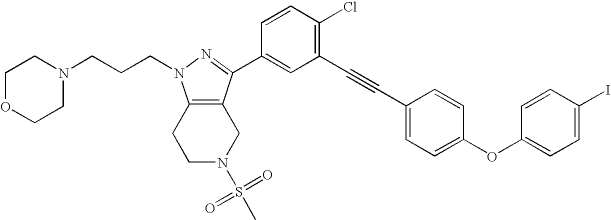

### Example 14

### 3-{4-Chloro-3-[4-(4-iodo-phenoxy)-phenylethynyl]-phenyl}-5-methanesulfonyl-1-(3-morpholin-4-yl-propyl)-4,5,6,7-tetrahydro-1H-pyrazolo[4,3-c]pyridine

MS (ESI): mass calcd. for C₃₄H₃₄CIIN₄O₄S, 757.1; m/z found, 758.2 [M+H]⁺.

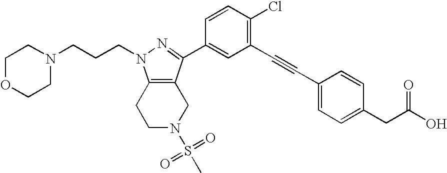

### Example 15

### (4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid, trifluoroacetic acid salt

To a solution of (4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester (19 mg, 0.031 mmol) in 2:1 EtOH/H₂O (0.9 mL) was added NaOH (3.7 mg, 0.093 mmol). The reaction mixture was stirred at rt for 30 min and then purified directly by reverse-phase HPLC (0.05% TFA/10-98% MeCN/H₂O) to afford the title compound as an off-white solid (11.7 mg, 53%). HPLC: Rₜ=4.75. MS (ESI): mass calcd. for C₃₀H₃₃ClN₄O₅S, 597.1; m/z found, 598.3 [M+H]⁺. ¹H NMR (CDCl₃): 7.74 (d, J=1.6, 1H), 7.56-7.53 (m, 2H), 7.46-7.43 (m, 2H), 7.32-7.29 (m, 2H), 4.50 (s, 2H), 4.12 (t, J=6.5, 2H), 3.95 (br m, 4H), 3.65-3.63 (m, 4H), 3.62 (br s, 1H), 3.15-3.09 (m, 2H), 2.92 (s, 3H), 2.82 (t, J=5.6, 4H), 2.39-2.31 (br m, 4H).

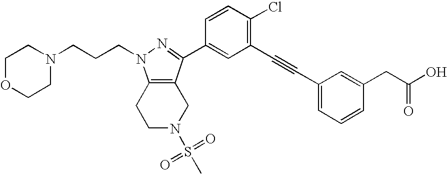

### Example 16

### (3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid

The title compound was prepared from (3-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester according to the method described in Example 15. MS (ESI): mass calcd. for C₃₀H₃₃ClN₄O₅S, 597.1; m/z found, 598.3 [M+H]

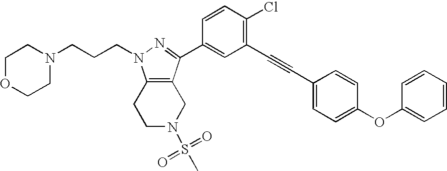

### Example 17

### 3-[4-Chloro-3-(4-phenoxy-phenylethynyl)-phenyl]-5-methanesulfonyl-1-(3-morpholin-4-yl-propyl)-4,5,6,7-tetrahydro-1H-pyrazolo[4,3-c]pyridine

Formic acid (12 μL) was added to a degassed solution of 3-{4-chloro-3-[4-(4-iodo-phenoxy)-phenylethynyl]-phenyl}-5-methanesulfonyl-1-(3-morpholin-4-yl-propyl)-4,5,6,7-tetrahydro-1H-pyrazolo[4,3-c]pyridine (119 mg, 0.157 mmol), Pd(OAc)₂ (3.6 mg, 0.016 mmol), PPh₃ (8.2 mg, 0.032 mmol), and TEA (66 μL, 0.473 mmol) in DMF (1.5 mL). The reaction mixture was heated at 60° C. under N₂ for 1.5 h. After cooling to rt, saturated (satd.) aq. NaHCO₃ was added, and the aqueous layer was extracted with CH₂Cl₂ (3×). The combined organic extracts were dried (Na₂SO₄), filtered, and concentrated to give a brown oil. Purification (SiO₂; 0-3% 2 M NH₃ in MeOH/CH₂Cl₂) afforded the title compound as a white solid (51 mg, 52%). MS (ESI): mass calcd. for C₃₄H₃₅ClN₄O₄S, 631.2; m/z found, 632.3 [M+H]⁺.

The compounds in Example 18-54 were prepared using methods analogous to those described for Intermediate 1, substituting the appropriate alkyne for TMSA in Step E.

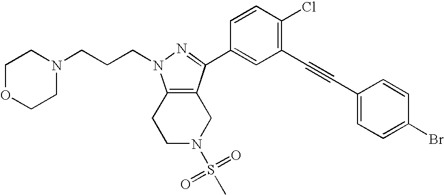

### Example 18

### 3-[3-(4-Bromo-phenylethynyl)-4-chloro-phenyl]-5-methanesulfonyl-1-(3-morpholin-4-yl-propyl)-4,5,6,7-tetrahydro-1H-pyrazolo[4,3-c]pyridine

HPLC: Rₜ=5.61. MS (ESI): mass calcd. for C₂₈H₃₀BrClN₄O₃S, 618.0; m/z found, 619.2 [M+H]⁺. ¹H NMR (CDCl₃): 7.79 (d, J=1.9, 1H), 7.53-7.44 (m, 6H), 4.54 (s, 2H), 4.10 (t, J=6.7, 2H), 3.70 (t, J=4.4, 4H), 3.66 (t, J=5.7, 2H), 2.91 (s, 3H), 2.91-2.88 (m, 2H), 2.32 (t, J=6.6, 2H).

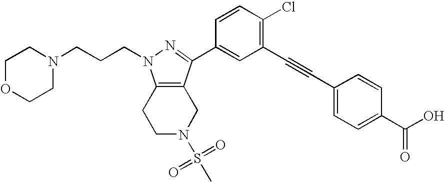

### Example 19

### 4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzoic acid

HPLC: Rₜ=4.66. MS (ESI): mass calcd. for C₂₉H₃₁ClN₄O₅S, 583.1; m/z found, 584.4 [M+H]⁺. ¹H NMR (CDCl₃): 8.06 (d, J=8.2, 2H), 7.80 (s, 1H), 7.67 (d, J=8.2, 2H), 7.54-7.50 (m, 2H), 4.54 (s, 2H), 4.28-4.18 (m, 2H), 4.06-4.00 (br m, 4H), 3.60 (t, J=5.7, 2H), 3.24-3.13 (m, 4H), 3.08-3.01 (m, 2H), 2.98 (s, 3H), 2.82-2.77 (m, 2H).

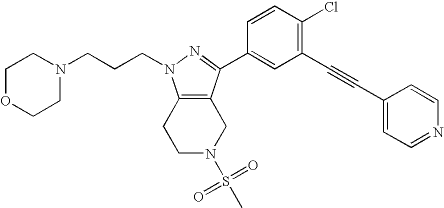

### Example 20

### 3-(4-Chloro-3-pyridin-4-ylethynyl-phenyl)-5-methanesulfonyl-1-(3-morpholin-4-yl-propyl)-4,5,6,7-tetrahydro-1H-pyrazolo[4,3-c]pyridine

HPLC: Rₜ=4.02. MS (ESI): mass calcd. for C₂₇H₃₀ClN₅O₃S, 540.1; m/z found, 541.3 [M+H]⁺. ¹H NMR (CDCl₃): 8.65-8.62 (m, 2H), 7.83 (d, J=2.0, 1H), 7.54 (dd, J=8.4, 2.1, 1H), 7.50-7.44 (m, 3H), 4.54 (s, 2H), 4.11 (t, J=6.8, 2H), 3.70 (t, J=4.5, 4H), 3.66 (t, J=5.8, 2H), 2.91 (s, 3H), 2.92-2.88 (m, 2H), 2.44-2.39 (m, 4H), 2.33 (t, J=6.8, 2H), 2.12-2.04 (m, 2H).

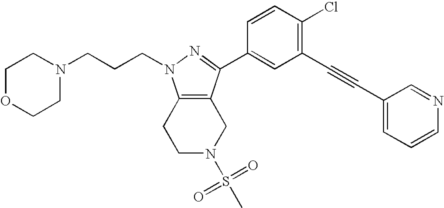

### Example 21

### 3-(4-Chloro-3-pyridin-3-ylethynyl-phenyl)-5-methanesulfonyl-1-(3-morpholin-4-yl-propyl)-4,5,6,7-tetrahydro-1H-pyrazolo[4,3-c]pyridine

HPLC: Rₜ=4.34. MS (ESI): mass calcd. for C₂₇H₃₀ClN₅O₃S, 540.1; m/z found, 541.3 [M+H]⁺. ¹H NMR (CDCl₃): 8.83 (s, 1H), 8.58 (d, J=3.9, 1H), 7.88 (dt, J=1.9, 7.9, 1H), 7.83 (d, J=2.0, 1H), 7.52 (dd, J=8.4, 2.1, 1H), 7.47 (d, J=8.4, 1H), 7.34-7.29 (m, 1H), 4.54 (s, 2H), 4.10 (t, J=6.8, 2H), 3.69 (t, J=4.6, 4H), 3.65 (t, J=5.8, 2H), 2.91 (s, 3H), 2.90 (t, J=5.8, 2H), 2.43-2.38 (br m, 4H), 2.32 (t, J=6.8, 2H), 2.11-2.04 (m, 2H).

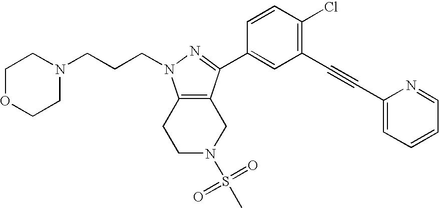

### Example 22

### 3-(4-Chloro-3-pyridin-2-ylethynyl-phenyl)-5-methanesulfonyl-1-(3-morpholin-4-yl-propyl)-4,5,6,7-tetrahydro-1H-pyrazolo[4,3-c]pyridine

HPLC: Rₜ=4.50. MS (ESI): mass calcd. for C₂₇H₃₀ClN₅O₃S, 540.1; m/z found, 541.3 [M+H]⁺. ¹H NMR (CDCl₃): 8.65 (d, J=4.5, 1H), 7.86 (d, J=2.1, 1H), 7.72 (dt, J=1.7, 7.7, 1H), 7.63-7.60 (m, 1H), 7.56 (dd, J=8.4, 2.1, 1H), 7.47 (d, J=8.4, 1H), 7.30-7.26 (m, 1H), 4.52 (s, 2H), 4.09 (t, J=6.8, 2H), 3.70 (t, J=4.6, 4H), 3.65 (t, J=5.8, 2H), 2.92 (s, 3H), 2.89 (t, J=5.7, 2H), 2.43-2.39 (br m, 4H), 2.33 (t, J=6.8, 2H), 2.11-2.03 (m, 2H).

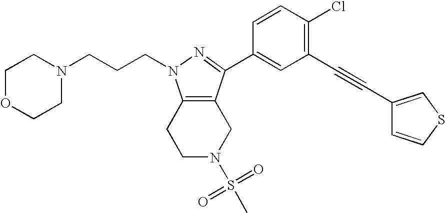

### Example 23

### 3-(4-Chloro-3-thiophen-3-ylethynyl-phenyl)-5-methanesulfonyl-1-(3-morpholin-4-yl-propyl)-4,5,6,7-tetrahydro-1H-pyrazolo[4,3-c]pyridine

HPLC: Rₜ=5.15. MS (ESI): mass calcd. for C₂₆H₂₉ClN₄O₃S₂, 545.1; m/z found, 546.3 [M+H]⁺. ¹H NMR (CDCl₃): 7.78 (d, J=1.9, 1H), 7.61 (dd, J=3.0, 1.1, 1H), 7.49 (dd, J=8.4, 2.1, 1H), 7.44 (d, J=8.4, 1H), 7.33 (dd, J=5.0, 3.0, 1H), 7.25 (dd, J=5.0, 1.1, 1H), 4.53 (s, 2H), 4.09 (t, J=6.8, 1H), 3.69 (t, J=4.5, 4H), 3.65 (t, J=5.8, 2H), 2.90 (s, 3H), 2.89 (t, J=5.7, 2H), 2.42-2.38 (br m, 4H), 2.32 (t, J=6.8, 2H), 2.10-2.03 (m, 2H).

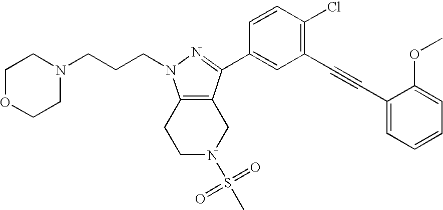

### Example 24

### 3-[4-Chloro-3-(2-methoxy-phenylethynyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.18. MS (ESI): mass calcd. for C₂₉H₃₃ClN₄O₄S, 569.1; m/z found, 570.3 [M+H]⁺. ¹H NMR (CDCl₃): 7.83 (d, J=1.5, 1H), 7.56 (dd, J=7.6, 1.7, 1H), 7.47-7.44 (m, 2H), 7.36-7.31 (m, 1H), 6.98-6.91 (m, 2H), 4.52 (s, 2H), 4.09 (t, J=6.8, 2H), 3.93 (s, 3H), 3.70 (t, J=4.6, 4H), 3.64 (t, J=5.8, 2H), 2.90 (s, 3H), 2.88 (t, J=5.7, 2H), 2.44-2.38 (br m, 4H), 2.33 (t, J=6.8, 2H), 2.11-2.03 (m, 2H).

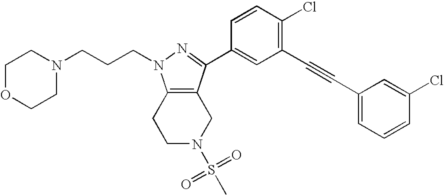

### Example 25

### 3-[4-Chloro-3-(3-chloro-phenylethynyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.60. MS (ESI): mass calcd. for C₂₈H₃₀Cl₂N₄O₃S, 573.6; m/z found, 574.3 [M+H]⁺. ¹H NMR (CDCl₃): 7.79 (d, J=2.0, 1H), 7.60-7.57 (m, 1H), 7.53-7.45 (m, 3H), 7.36-7.28 (m, 2H), 4.54 (s, 2H), 4.10 (t, J=6.8, 2H), 3.70 (t, J=4.6, 4H), 3.66 (t, J=5.8, 2H), 2.91 (s, 3H), 2.90 (t, J=5.7, 2H), 2.43-2.39 (br m, 4H), 2.33 (t, J=6.8, 2H), 2.12-2.05 (m, 2H).

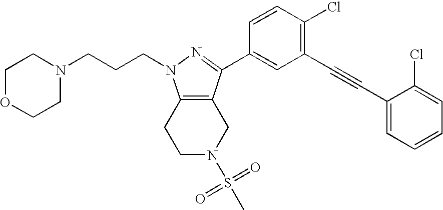

### Example 26

### 3-[4-Chloro-3-(2-chloro-phenylethynyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.48. MS (ESI): mass calcd. for C₂₈H₃₀Cl₂N₄O₃S, 573.6; m/z found, 574.3 [M+H]⁺. ¹H NMR (CDCl₃): 7.85 (d, J=1.8, 1H), 7.65-7.62 (m, 1H), 7.50-7.43 (m, 3H), 7.32-7.27 (m, 2H), 4.53 (s, 2H), 4.15-4.08 (m, 2H), 3.70 (t, J=4.6, 4H), 3.65 (t, J=5.8, 2H), 2.91-2.87 (m, 2H), 2.90 (s, 3H), 2.43-2.39 (br m, 4H), 2.33 (t, J=6.8, 2H), 2.11-2.04 (m, 2H).

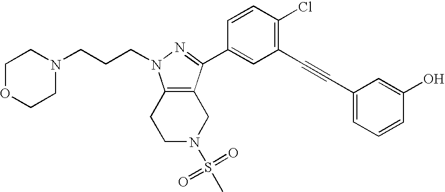

### Example 27

### 3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol

HPLC: Rₜ=4.80. MS (ESI): mass calcd. for C₂₈H₃₁ClN₄O₄S, 555.1; m/z found, 556.6 [M+H]⁺. ¹H NMR (CDCl₃): 7.69 (d, J=2.1, 1H), 7.45 (dd, J=8.5, 2.1, 1H), 7.36 (d, J=8.4, 1H), 7.18 (t, J=7.9, 1H), 7.10-7.06 (m, 1H), 6.88-6.87 (m, 1H), 6.81-6.78 (m, 1H), 4.47 (s, 2H), 4.07 (t, J=6.7, 2H), 3.71 (t, J=4.5, 4H), 3.54 (t, J=5.7, 2H), 2.88 (s, 3H), 2.77 (t, J=5.5, 2H), 2.48-2.43 (br m, 4H), 2.38 (t, J=7.1, 2H), 2.09-2.01 (m, 2H).

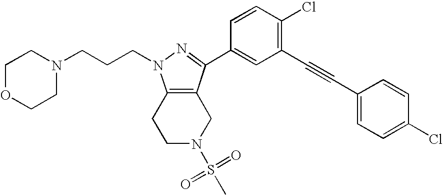

### Example 28

### 3-[4-Chloro-3-(4-chloro-phenylethynyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.56. MS (ESI): mass calcd. for C₂₈H₃₀Cl₂N₄O₃S, 573.6; m/z found, 574.3 [M+H]⁺. ¹H NMR (CDCl₃): 7.79 (d, J=2.0, 1H), 7.54-7.48 (m, 2H), 7.46 (d, J=8.4, 2H), 7.37-7.33 (m, 2H), 4.53 (s, 2H), 4.10 (t, J=6.8, 2H), 3.70 (t, J=4.6, 4H), 3.65 (t, J=5.8, 2H), 2.90 (s, 3H), 2.89 (t, J=5.9, 2H), 2.42-2.38 (br m, 4H), 2.32 (t, J=6.9, 2H), 2.11-2.04 (m, 2H).

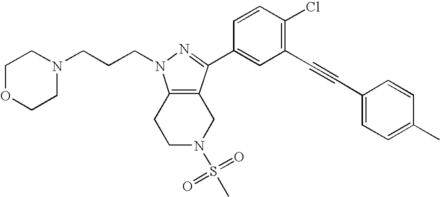

### Example 29

### 3-(4-Chloro-3-p-tolylethynyl-phenyl)-5-methanesulfonyl-1-(3-morpholin-4-yl-propyl)-4,5,6,7-tetrahydro-1H-pyrazolo[4,3-c]pyridine

MS (ESI): mass calcd. for C₂₉H₃₃ClN₄O₃S, 553.1; m/z found, 554.6 [M+H]⁺.

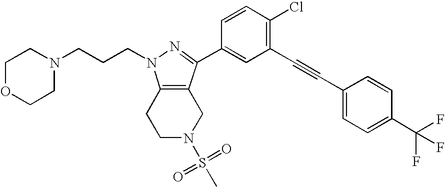

### Example 30

### 3-[4-Chloro-3-(4-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₉H₃₀ClF₃N₄O₃S, 607.1; m/z found, 608.3 [M+H]⁺.

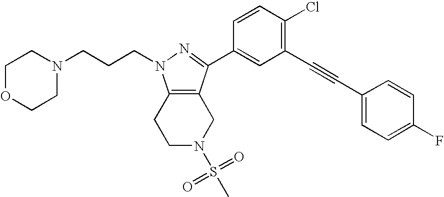

### Example 31

### 3-[4-Chloro-3-(4-fluoro-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₈H₃₀ClFN₄O₃S, 557.1; m/z found, 558.3 [M+H]⁺.

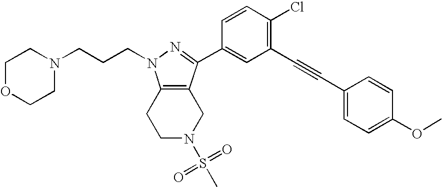

### Example 32

### 3-[4-Chloro-3-(4-methoxy-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₉H₃₃ClN₄O₄S, 569.1; m/z found, 570.3 [M+H]⁺.

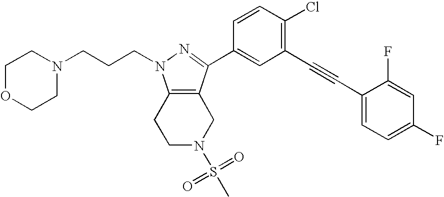

### Example 33

### 3-[4-Chloro-3-(2,4-difluoro-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₈H₂₉ClF₂N₄O₃S, 575.1; m/z found, 576.3 [M+H]⁺.

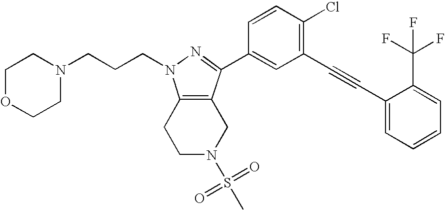

### Example 34

### 3-[4-Chloro-3-(2-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₉H₃₀ClF₃N₄O₃S, 607.1; m/z found, 608.3 [M+H]⁺.

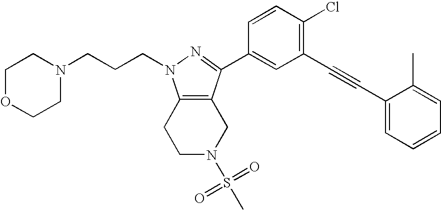

### Example 35

### 3-(4-Chloro-3-o-tolylethynyl-phenyl)-5-methanesulfonyl-1-(3-morpholin-4-yl-propyl)-4,5,6,7-tetrahydro-1H-pyrazolo[4,3-c]pyridine

MS (ESI): mass calcd. for C₂₉H₃₃ClN₄O₃S, 553.1; m/z found, 554.6 [M+H]⁺.

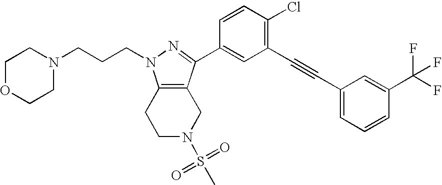

### Example 36

### 3-[4-Chloro-3-(3-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₉H₃₀ClF₃N₄O₃S, 607.1; m/z found, 608.3 [M+H]⁺.

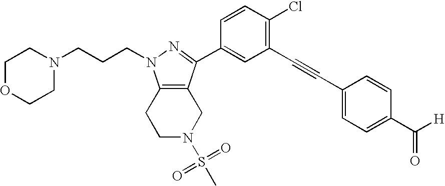

### Example 37

### 4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzaldehyde

MS (ESI): mass calcd. for C₂₉H₃₁ClN₄O₄S, 567.1; m/z found, 568.3 [M+H]⁺.

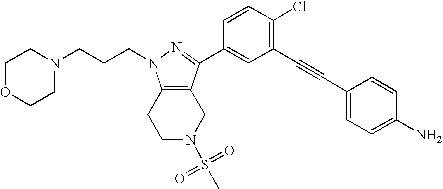

### Example 38

### 4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenylamine

HPLC: Rₜ=4.47. MS (ESI): mass calcd. for C₂₈H₃₂ClN₅O₃S, 554.1; m/z found, 555.4 [M+H]⁺. ¹H NMR (CDCl₃): 7.74 (s, 1H), 7.43-7.36 (m, 4H), 6.64 (d, J=8.5, 2H), 4.50 (s, 2H), 4.09 (t, J=6.9, 2H), 3.93 (br s, 2H), 3.72 (t, J=4.3, 4H), 3.62 (t, J=5.6, 2H), 2.89 (s, 3H), 2.88-2.84 (m, 2H), 2.50-2.45 (br m, 4H), 2.39 (t, J=6.8, 2H), 2.14-2.06 (m, 2H).

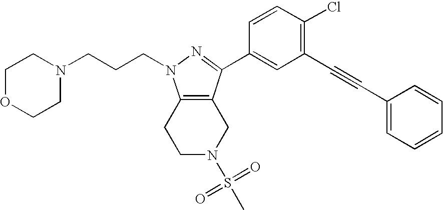

### Example 39

### 3-(4-Chloro-3-phenylethynyl-phenyl)-5-methanesulfonyl-1-(3-morpholin-4-yl-propyl)-4,5,6,7-tetrahydro-1H-pyrazolo[4,3-c]pyridine

HPLC: Rₜ=5.19. MS (ESI): mass calcd. for C₂₈H₃₁ClN₃O₃S, 539.1; m/z found, 540.4 [M+H]⁺. ¹H NMR (CDCl₃): 7.80 (d, J=1.7, 1H), 7.61-7.59 (m, 2H), 7.47-7.44 (m, 2H), 7.38-7.36 (m, 3H), 4.52 (s, 2H), 4.10 (t, J=6.8, 2H), 3.69 (t, J=4.4, 4H), 3.64 (t, J=5.7, 2H), 2.89-2.87 (m, 5H), 2.40-2.39 (m, 4H), 2.32 (t, J=1.7, 2H), 2.08-2.05 (m, 2H).

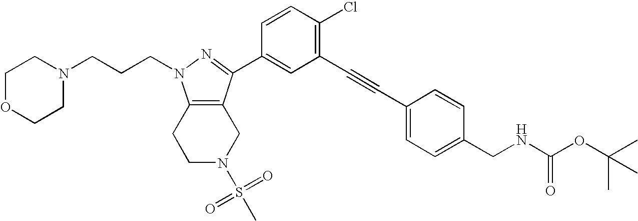

### Example 40

### (4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-carbamic acid tert-butyl ester

MS (ESI): mass calcd. for C₃₄H₄₂ClN₅O₅S, 668.3; m/z found, 669.6 [M+H]⁺.

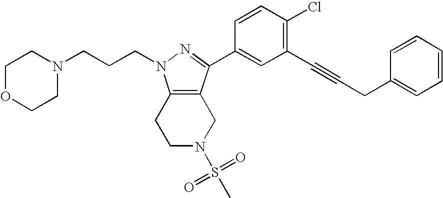

### Example 41

### 3-[4-Chloro-3-(3-phenyl-prop-1-ynyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.20. MS (ESI): mass calcd. for C₂₉H₃₃ClN₄O₃S, 553.1; m/z found, 554.4 [M+H]⁺. ¹H NMR (CDCl₃): 7.71 (d, J=1.9, 1H), 7.49-7.40 (m, 4H), 7.38-7.33 (m, 2H), 7.28-7.23 (m, 1H), 4.49 (s, 2H), 4.07 (t, J=6.8, 2H), 3.92 (s, 2H), 3.69 (t, J=4.4, 4H), 3.62 (t, J=5.7, 2H), 2.87 (s, 3H), 2.86 (t, J=5.4, 2H), 2.44-2.39 (br m, 4H), 2.33 (t, J=6.8, 2H), 2.10-2.03 (m, 2H).

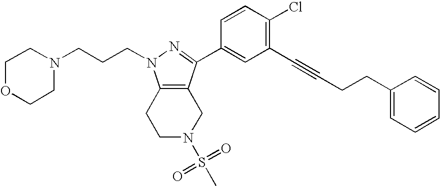

### Example 42

### 3-[4-Chloro-3-(4-phenyl-but-1-ynyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.35. MS (ESI): mass calcd. for C₃₀H₃₅ClN₄O₃S, 567.2; m/z found, 568.3 [M+H]⁺. ¹H NMR (CDCl₃): 7.63 (d, J=2.1, 1H), 7.47-7.44 (m, 2H), 7.39 (d, J=8.4, 1H), 7.33-7.30 (m, 3H), 7.25-7.20 (m, 1H), 4.50 (s, 2H), 4.09 (t, J=6.8, 2H), 3.69 (t, J=4.5, 4H), 3.64 (t, J=5.8, 2H), 2.98 (t, J=7.5, 2H), 2.89 (s, 3H), 2.88 (t, J=5.7, 2H), 2.78 (t, J=7.5, 2H), 2.43-2.38 (br m, 4H), 2.32 (t, J=6.8, 2H), 2.10-2.03 (m, 2H).

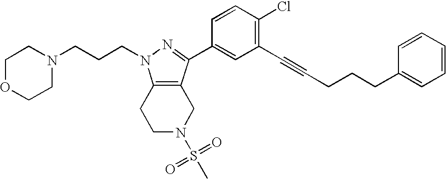

### Example 43

### 3-[4-Chloro-3-(5-phenyl-pent-1-ynyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.58. MS (ESI): mass calcd. for C₃₁H₃₇ClN₄O₃S, 581.2; m/z found, 582.4 [M+H]⁺. ¹H NMR (CDCl₃): 7.67 (d, J=2.0, 1H), 7.45 (dd, J=8.4, 2.1, 1H), 7.40 (d, J=8.4, 1H), 7.22-7.17 (m, 1H), 4.49 (s, 2H), 4.08 (t, J=6.8, 2H), 3.68 (t, J=4.6, 4H), 3.62 (t, J=5.8, 2H), 2.88-2.82 (m, 4H), 2.87 (s, 3H), 2.49 (t, J=6.9, 2H), 2.41-2.37 (br m, 4H), 2.31 (t, J=6.8, 2H), 2.09-2.01 (m, 2H), 2.01-1.92 (m, 2H).

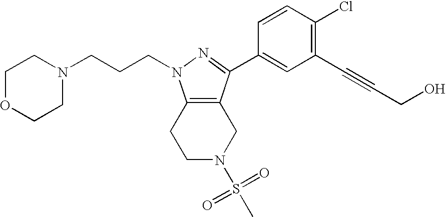

### Example 44

### 3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-yn-1-ol

MS (ESI): mass calcd. for C₂₃H₂₉ClN₄O₄S, 493.0; m/z found, 494.4 [M+H]⁺.

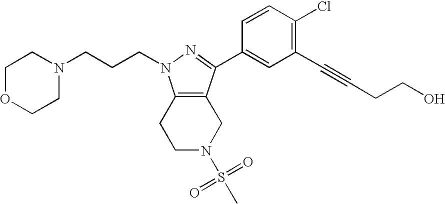

### Example 45

### 4-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-but-3-yn-1-ol

MS (ESI): mass calcd. for C₂₄H₃₁ClN₄O₄S, 507.1; m/z found, 508.4 [M+H]⁺.

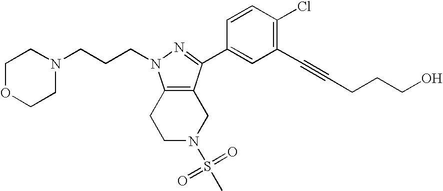

### Example 46

### 5-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-pent-4-yn-1-ol

MS (ESI): mass calcd. for C₂₅H₃₃ClN₄O₄S, 521.1; m/z found, 522.4 [M+H]⁺.

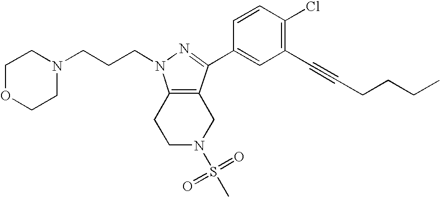

### Example 47

### 3-(4-Chloro-3-hex-1-ynyl-phenyl)-5-methanesulfonyl-1-(3-morpholin-4-yl-propyl)-4,5,6,7-tetrahydro-1H-pyrazolo[4,3-c]pyridine

HPLC: Rₜ=5.33. MS (ESI): mass calcd. for C₂₆H₃₅ClN₄O₃S, 519.1; m/z found, 520.4 [M+H]⁺. ¹H NMR (CDCl₃): 7.65 (d, J=2.0, 1H), 7.43 (dd, J=8.4, 2.1, 1H), 7.39 (d, J=8.4, 1H), 4.50 (s, 2H), 4.08 (t, J=6.8, 2H), 3.69 (t, J=4.6, 4H), 3.64 (t, J=5.8, 2H), 2.89 (s, 3H), 2.88 (t, J=5.8, 2H), 2.49 (t, J=7.0, 2H), 2.42-2.38 (br m, 4H), 2.32 (t, J=6.8, 2H), 2.09-2.02 (m, 2H), 1.68-1.60 (m, 2H), 1.58-1.48 (m, 2H), 0.96 (t, J=7.3, 3H).

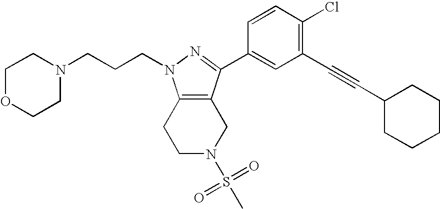

### Example 48

### 3-(4-Chloro-3-cyclohexylethynyl-phenyl)-5-methanesulfonyl-1-(3-morpholin-4-yl-propyl)-4,5,6,7-tetrahydro-1H-pyrazolo[4,3-c]pyridine

HPLC: Rₜ=5.61. MS (ESI): mass calcd. for C₂₈H₃₇ClN₄O₃S, 545.2; m/z found, 546.4 [M+H]⁺. ¹H NMR (CDCl₃): 7.67-7.65 (m, 1H), 7.41-7.40 (m, 2H), 4.51 (s, 2H), 4.09 (t, J=6.8, 2H), 3.70 (t, J=4.6, 4H), 3.65 (t, J=5.8, 2H), 2.89 (s, 3H), 2.88 (t, J=5.8, 2H), 2.73-2.66 (m, 1H), 2.44-2.38 (br m, 4H), 2.32 (t, J=6.8, 2H), 2.10-2.03 (m, 2H), 1.94-1.86 (m, 2H), 1.84-1.75 (m, 2H), 1.66-1.52 (m, 3H), 1.44-1.35 (m, 3H).

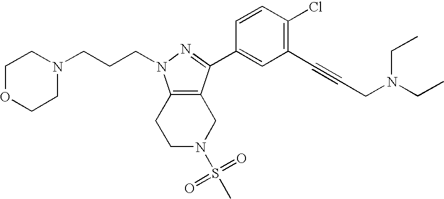

### Example 49

### (3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-diethyl-amine

HPLC: Rₜ=3.98. MS (ESI): mass calcd. for C₂₇H₃₈ClN₅O₃S, 548.2; m/z found, 549.4 [M+H]⁺. ¹H NMR (CDCl₃): 7.61 (d, J=2.0, 1H), 7.39 (dd, J=8.4, 2.1, 1H), 7.34 (d, J=8.4, 1H), 4.43 (s, 2H), 4.02 (t, J=6.8, 2H), 3.66 (s, 2H), 3.62 (t, J=4.6, 4H), 3.57 (t, J=5.8, 2H), 2.82 (s, 3H), 2.81 (t, J=5.9, 2H), 2.61 (q, J=7.2, 4H), 2.35-2.31 (br m, 4H), 2.25 (t, J=6.8, 2H), 2.03-1.96 (m, 2H), 1.07 (t, J=7.2, 6H).

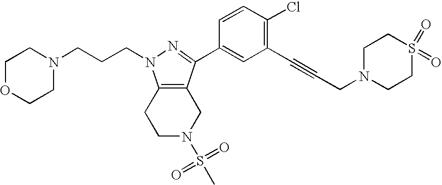

### Example 50

### 3-{4-Chloro-3-[3-(1,1-dioxo-1 A⁶-thiomorpholin-4-yl)-prop-1-ynyl]-phenyl}-5-methanesulfonyl-1-(3-morpholin-4-yl-propyl)-4,5,6,7-tetrahydro-1H-pyrazolo[4,3-c]pyridine

MS (ESI): mass calcd. for C₂₇H₃₆ClN₅O₅S₂, 610.2; m/z found, 611.3 [M+H]⁺.

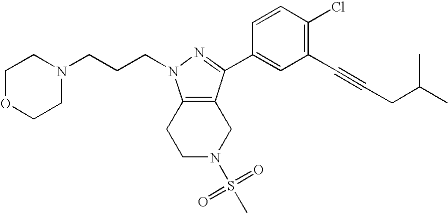

### Example 51

### 3-[4-Chloro-3-(4-methyl-pent-1-ynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₆H₃₅ClN₄O₃S, 519.1; m/z found, 520.4 [M+H]⁺.

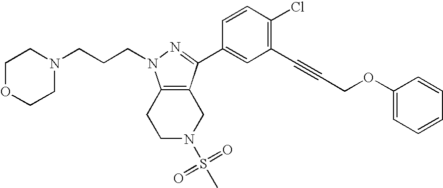

### Example 52

### 3-[4-Chloro-3-(3-phenoxy-prop-1-ynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₂₉H₃₃ClN₄O₄S, 569.1; m/z found, 570.3 [M+H]⁺.

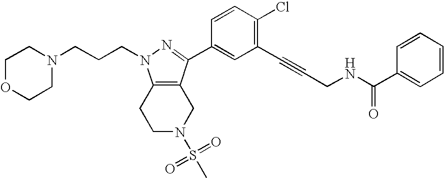

### Example 53

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzamide

MS (ESI): mass calcd. for C₃₀H₃₄ClN₅O₄S, 596.2; m/z found, 597.4 [M+H]⁺.

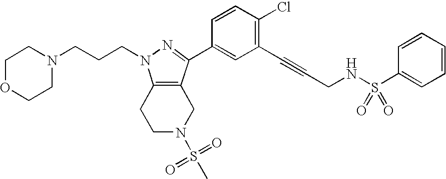

### Example 54

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl]-prop-2-ynyl)-benzenesulfonamide

MS (ESI): mass calcd. for C₂₉H₃₄ClN₅O₅S₂, 632.2; m/z found, 633.4 [M+H]⁺.

The compounds in Examples 55-65 were prepared using methods analogous to those described in Example 2. The alkynes used as starting materials for Examples 55-65 are described in the preceding examples.

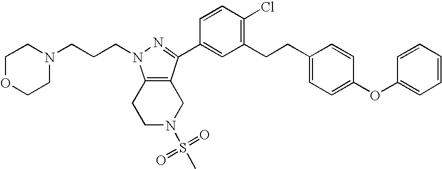

### Example 55

### 3-{4-Chloro-3-[2-(4-phenoxy-phenyl)-ethyl]-phenyl}-5-methanesulfonyl-1-(3-morpholin-4-yl-propyl)-4,5,6,7-tetrahydro-1H-pyrazolo[4,3-c]pyridine

MS (ESI): mass calcd. for C₃₄H₃₉ClN₄O₄S, 635.2; m/z found, 636.4 [M+H]⁺.

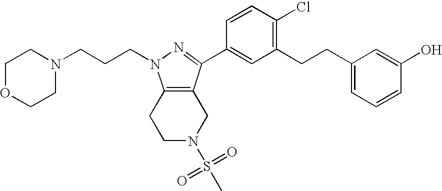

### Example 56

### 3-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenol

MS (ESI): mass calcd. for C₂₈H₃₅ClN₄O₄S, 559.1; m/z found, 560.4 [M+H]⁺.

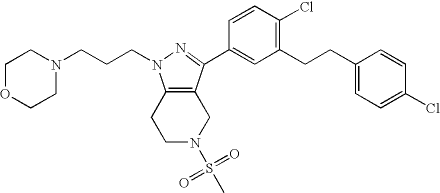

### Example 57

### 3-{4-Chloro-3-[2-(4-chloro-phenyl)-ethyl]-phenyl}-5-methanesulfonyl-1-(3-morpholin-4-yl-propyl)-4,5,6,7-tetrahydro-1H-pyrazolo[4,3-c]pyridine

HPLC: Rₜ=5.89. MS (ESI): mass calcd. for C₂₈H₃₄Cl₂N₄O₃S, 577.6; m/z found, 578.3 [M+H]⁺. ¹H NMR (CDCl₃): 7.42-7.38 (m, 2H), 7.33 (dd, J=8.3, 2.1, 1H), 7.27-7.23 (m, 2H), 7.17-7.13 (m, 2H), 4.44 (s, 2H), 4.10 (t, J=6.8, 2H), 3.70 (t, J=4.6, 2H), 3.65 (t, J=5.8, 2H), 3.06-3.01 (m, 2H), 2.94-2.87 (m, 4H), 2.89 (s, 3H), 2.43-2.37 (br m, 4H), 2.32 (t, J=6.8, 2H), 2.10-2.03 (m, 2H).

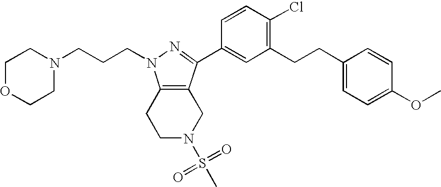

### Example 58

### 3-{4-Chloro-3-[2-(4-methoxy-phenyl)-ethyl]-phenyl}-5-methanesulfonyl-1-(3-morpholin-4-yl-propyl)-4,5,6,7-tetrahydro-1H-pyrazolo[4,3-c]pyridine

HPLC: Rₜ=5.22. MS (ESI): mass calcd. for C₂₉H₃₇ClN₄O₄S, 573.2; m/z found, 574.4 [M+H]⁺. ¹H NMR (CDCl₃): 7.46 (d, J=2.0, 1H), 7.39 (d, J=8.3, 1H), 7.33 (dd, J=8.3, 2.1, 1H), 7.18-7.14 (m, 2H), 6.87-6.83 (m, 2H), 4.45 (s, 2H), 4.10 (t, J=6.8, 2H), 3.79 (s, 3H), 3.70 (t, J=4.6, 4H), 3.64 (t, J=5.8, 2H), 3.06-3.01 (m, 2H), 2.91-2.87 (m, 4H), 2.88 (s, 3H), 2.44-2.40 (br m, 4H), 2.34 (t, J=6.9, 2H), 2.11-2.03 (m, 2H).

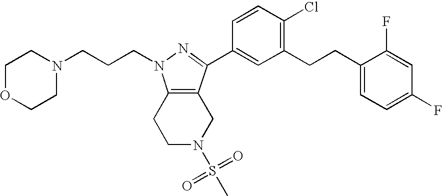

### Example 59

### 3-{4-Chloro-3-[2-(2,4-difluoro-phenyl)-ethyl]-phenyl}-5-methanesulfonyl-1-(3-morpholin-4-yl-propyl)-4,5,6,7-tetrahydro-1H-pyrazolo[4,3-c]pyridine

HPLC: Rₜ=5.80. MS (ESI): mass calcd. for C₂₈H₃₃ClF₂N₄O₃S, 579.1; m/z found, 580.3 [M+H]⁺. ¹H NMR (CDCl₃): 7.43 (d, J=2.0, 1H), 7.39 (d, J=8.3, 1H), 7.33 (dd, J=8.3, 2.1, 1H), 7.15-7.08 (m, 1H), 6.82-6.76 (m, 2H), 4.45 (s, 2H), 4.10 (t, J=6.8, 2H), 3.71 (t, J=4.6, 4H), 3.65 (t, J=5.8, 2H), 3.06-3.01 (m, 2H), 2.97-2.91 (m, 2H), 2.90-2.86 (m, 2H), 2.89 (s, 3H), 2.45-2.41 (br m, 4H), 2.35 (t, J=6.9, 2H), 2.11-2.05 (m, 2H).

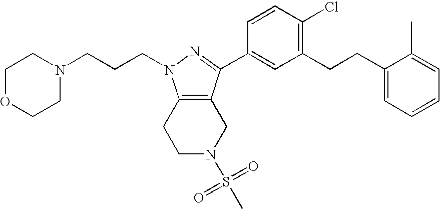

### Example 60

### 3-[4-Chloro-3-(2-o-tolyl-ethyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.40. MS (ESI): mass calcd. for C₂₉H₃₇ClN₄O₃S, 557.2; m/z found, 558.5 [M+H]⁺. ¹H NMR (CDCl₃): 7.49 (d, J=2.0, 1H), 7.40 (d, J=8.3, 1H), 7.33 (dd, J=8.3, 2.1, 1H), 7.22-7.11 (m, 3H), 4.44 (s, 2H), 4.09 (t, J=6.8, 2H), 3.69 (t, J=4.6, 4H), 3.63 (t, J=5.8, 2H), 3.04-2.99 (m, 2H), 2.96-2.86 (m, 4H), 2.87 (s, 3H), 2.43-2.38 (br m, 4H), 2.36 (s, 3H), 2.32 (t, J=6.6, 2H), 2.10-2.03 (m, 2H).

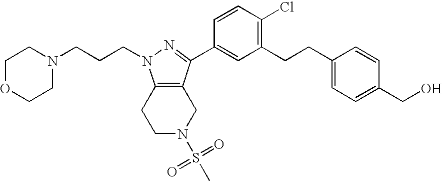

### Example 61

### [4-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenyl]-methanol

HPLC: Rₜ=4.70. MS (ESI): mass calcd. for C₂₉H₃₇ClN₄O₄S, 573.2; m/z found, 574.2 [M+H]⁺. ¹H NMR (CDCl₃): 7.39 (d, J=8.2, 1H), 7.37-7.32 (m, 2H), 7.27 (d, J=8.1, 2H), 7.19 (d, J=8.0, 2H), 4.63 (s, 2H), 4.39 (s, 2H), 4.07 (t, J=6.8, 2H), 3.67 (t, J=4.6, 4H), 3.63 (t, J=5.8, 2H), 3.10-3.02 (m, 2H), 2.98-2.92 (m, 2H), 2.89-2.83 (m, 2H), 2.86 (s, 3H), 2.45 (br s, 1H), 2.42-2.36 (br m, 4H), 2.31 (t, J=6.9, 2H), 2.10-2.01 (m, 2H).

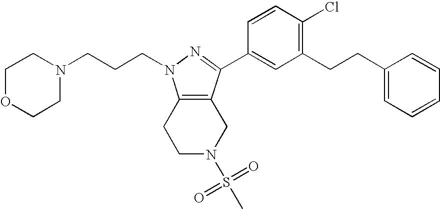

### Example 62

### 3-(4-Chloro-3-phenethyl-phenyl)-5-methanesulfonyl-1-(3-morpholin-4-yl-propyl)-4,5,6,7-tetrahydro-1H-pyrazolo[4,3-c]pyridine

HPLC: Rₜ=5.24. MS (ESI): mass calcd. for C₂₈H₃₅ClN₄O₃S, 543.1; m/z found, 544.5 [M+H]⁺. ¹H NMR (CDCl₃): 7.48 (d, J=2.0, 1H), 7.39 (d, J=8.3, 1H), 7.32 (dd, J=8.1, 2.2, 2H), 7.31-7.18 (m, 5H), 4.44 (s, 2H), 4.09 (t, J=6.7, 2H), 3.69 (t, J=4.6, 4H), 3.64 (t, J=5.7, 2H), 3.09-3.04 (m, 2H), 2.97-2.92 (m, 2H), 2.89-2.86 (m, 2H), 2.87 (s, 3H), 2.43-2.38 (br m, 4H), 2.32 (t, J=6.8, 2H), 2.10-2.03 (m, 2H).

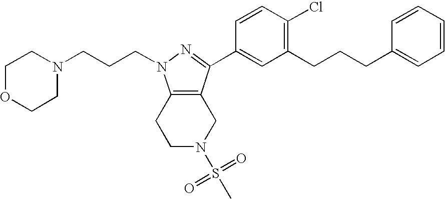

### Example 63

### 3-[4-Chloro-3-(3-phenyl-propyl)-phenyl]-5-methanesulfonyl-1-(3-morpholin-4-yl-propyl)-4,5,6,7-tetrahydro-1H-pyrazolo[4,3-c]pyridine

HPLC: Rₜ=5.42. MS (ESI): mass calcd. for C₂₉H₃₇ClN₄O₃S, 557.2; m/z found, 558.3 [M+H]⁺. ¹H NMR (CDCl₃): 7.51 (d, J=2.0, 1H), 7.36 (d, J=8.3, 1H), 7.31-7.26 (m, 4H), 7.23-7.17 (m, 2H), 4.49 (s, 2H), 4.09 (t, J=6.8, 2H), 3.69 (t, J=4.6, 4H), 3.64 (t, J=5.8, 2H), 2.90-2.85 (m, 2H), 2.86 (s, 3H), 2.84-2.78 (m, 2H), 2.72 (t, J=7.8, 2H), 2.42-2.38 (m, 4H), 2.32 (t, J=6.8, 2H), 2.09-1.95 (m, 4H).

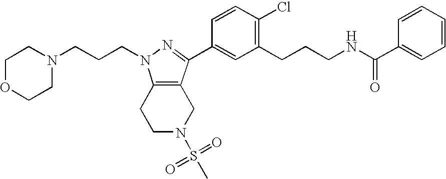

### Example 64

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzamide

MS (ESI): mass calcd. for C₃₀H₃₈ClN₅O₄S, 600.2; m/z found, 601.5 [M+H]⁺.

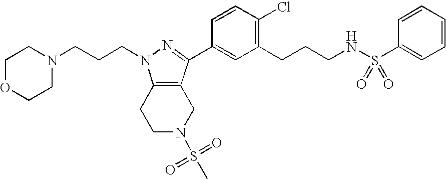

### Example 65

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide

HPLC: Rₜ=4.82. MS (ESI): mass calcd. for C₂₉H₃₈ClN₅O₅S₂, 636.2; m/z found, 637.4 [M+H]⁺. ¹H NMR (CDCl₃): 7.87-7.84 (m, 2H), 7.59-7.54 (m, 1H), 7.52-7.47 (m, 2H), 7.44 (d, J=1.7, 1H), 7.37-7.31 (m, 2H), 4.87 (t, J=6.1, 1H), 4.51 (s, 2H), 4.09 (t, J=6.8, 2H), 3.69 (t, J=4.6, 4H), 3.64 (t, J=5.8, 2H), 2.99 (q, J=6.5, 2H), 2.91 (s, 3H), 2.89 (t, J=5.8, 2H), 2.79-2.74 (m, 2H), 2.43-2.38 (m, 4H), 2.32 (t, J=6.8, 2H), 2.10-2.03 (m, 2H), 1.86-1.78 (m, 2H).

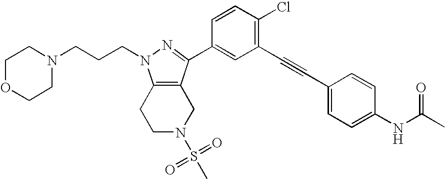

### Example 66

### N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetamide

To a solution of 4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenylamine (80 mg, 0.144 mmol) and pyridine (28 μL, 0.347 mmol) in CH₂Cl₂ (1.4 mL) was added Ac₂O (17 μL, 0.173 mmol) dropwise. The reaction mixture was stirred at rt for 14 h and transferred directly to a silica gel column. Purification (SiO₂; 0-10% 2 M NH₃ in MeOH/CH₂Cl₂) afforded the title compound as a clear oil (65 mg, 76%). HPLC: Rₜ=5.90. MS (ESI): mass calcd. for C₃₀H₃₄ClN₅O₄S, 596.2; m/z found, 597.4 [M+H]⁺. ¹H NMR (CDCl₃): 7.78 (d, J=1.8, 1H), 7.72 (s, 1H), 7.57-7.51 (m, 4H), 7.48-7.42 (m, 2H), 4.52 (s, 2H), 4.09 (t, J=6.8, 2H), 3.69 (t, J=4.6, 4H), 3.64 (t, J=5.8, 2H), 2.90 (s, 3H), 2.90-2.86 (m, 2H), 2.42-2.38 (m, 4H), 2.32 (t, J=6.8, 2H), 2.18 (s, 3H), 2.10-2.04 (m, 2H).

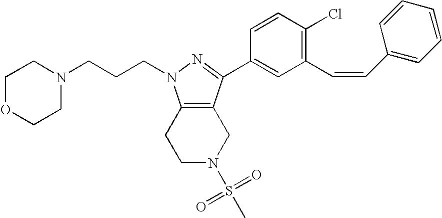

### Example 67

### 3-(4-Chloro-3-Z-styryl-phenyl)-5-methanesulfonyl-1-(3-morpholin-4-yl-propyl)-4,5,6,7-tetrahydro-1H-pyrazolo[4,3-c]pyridine

To a pressure tube containing a mixture of 3-(4-chloro-3-phenylethynyl-phenyl)-5-methanesulfonyl-1-(3-morpholin-4-yl-propyl)-4,5,6,7-tetrahydro-1H-pyrazolo[4,3-c]pyridine (100 mg, 0.185 mmol), and Lindlar's catalyst (100 mg) in EtOAc (6 mL) was added quinoline (75 mg). The tube was placed in a shaker under 15 psi of H₂ for 5 h. The reaction mixture was filtered through diatomaceous earth and concentrated to give a yellow oil. Purification (SiO₂; 0-3% 2 M NH₃ in MeOH/CH₂Cl₂) provided the title compound as a clear oil. The compound was obtained and analyzed as a 1.5:1 mixture of atropisomers at rt. HPLC: Rₜ=5.11 (major), 5.24 (minor). MS (ESI): mass calcd. for C₂₈H₃₃ClN₄O₃S, 541.1; m/z found, 542.3 [M+H]⁺. Major atropisomer: ¹H NMR (CDCl₃): 7.81-7.15 (m, 4.8H), 6.73 (d, J=12.2, 0.6H), 6.68 (d, J=12.2, 0.6H), 4.04 (s, 1.2H), 4.02 (t, J=6.8, 1.2H), 3.67 (t, J=4.7, 2.4H), 3.53 (t, J=5.8, 1.2H), 2.79 (t, J=5.7, 1.2H), 2.77 (s, 1.8H), 2.39-2.34 (br m, 2.4H), 2.28 (t, J=6.9, 1.2H), 2.04-1.96 (m, 1.2H). Minor atropisomer: ¹H NMR (CDCl₃): 7.81-7.15 (m, 4H), 4.53 (s, 0.8H), 4.10 (t, J=6.8, 0.8H), 3.70 (t, J=4.5, 1.6H), 3.64 (t, J=5.7, 0.8H), 2.90 (s, 1.2H), 2.88 (t, J=4.4, 0.8H), 2.43-2.39 (br m, 1.6H), 2.33 (t, J=6.7, 0.8H), 2.11-2.04 (m, 0.8H).

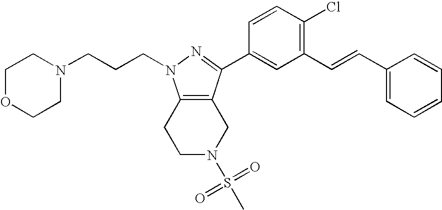

### Example 68

### 3-(4-Chloro-3-E-styryl-phenyl)-5-methanesulfonyl-1-(3-morpholin-4-yl-propyl)-4,5,6,7-tetrahydro-1H-pyrazolo[4,3-c]pyridine

A solution of 3-(4-chloro-3-iodo-phenyl)-5-methanesulfonyl-1-(3-morpholin-4-yl-propyl)-4,5,6,7-tetrahydro-1H-pyrazolo[4,3-c]pyridine (100 mg, 0.177 mmol), trans-cinnamylboronic acid (29 mg, 0.195 mmol), PdCl₂(dppf)-CH₂Cl₂ (14.5 mg, 0.018 mmol), and K₃PO₄ (45 mg, 0.212 mmol) in degassed DMF (1.0 mL) was heated at 50° C. under N₂ for 16 h. After cooling to rt, the reaction mixture was diluted with H₂O and extracted with CH₂Cl₂ (2×). The combined organic extracts were washed with brine, dried (Na₂SO₄), filtered, and concentrated to give a brown oil. Purification (SiO₂; 0-3% 2 M NH₃ in MeOH/CH₂Cl₂) provided the title compound as a clear oil (57 mg, 60%). HPLC: Rₜ=5.29. MS (ESI): mass calcd. for C₂₈H₃₃ClN₄O₃S, 541.1; m/z found, 542.3 [M+H]⁺. ¹H NMR (CDCl₃): 7.97 (d, J=2.0, 1H), 7.59-7.56 (m, 2H), 7.52 (d, J=16.3, 1H), 7.46-7.28 (m, 5H), 7.16 (d, J=16.3, 1H), 4.55 (s, 2H), 4.13 (t, J=6.9, 2H), 3.71 (t, J=4.6, 4H), 3.67 (t, J=5.8, 2H), 2.91 (t, J=5.8, 2H), 2.89 (s, 3H), 2.45-2.40 (br m, 4H), 2.35 (t, J=6.8, 2H), 2.13-2.05 (m, 2H).

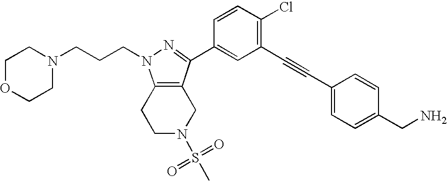

### Example 69

### 4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzylamine

To a solution of (4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-carbamic acid tert-butyl ester (622 mg, 0.931 mmol) in CH₂Cl₂ (4.7 mL) was added TFA (1.2 mL). The reaction mixture was stirred at rt for 1 h and then concentrated. Purification (SiO₂; 0-5% 2 M NH₃ in MeOH/CH₂Cl₂) afforded the title compound as a white foam (400 mg, 76%). MS (ESI): mass calcd. for C₂₉H₃₄ClN₅O₃S, 568.1; m/z found, 569.5 [M+H]⁺.

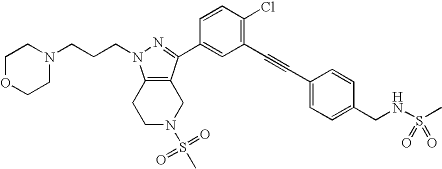

### Example 70

### N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methanesulfonamide

To a solution of 4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzylamine (70 mg, 0.123 mmol) and pyridine (22 μL, 0.272 mmol) in CH₂Cl₂ (1.2 mL) was added MsCl (11 μL, 0.136 mmol). The reaction mixture was stirred at rt for 16 h and transferred directly to a silica gel column. Purification (SiO₂; 0-3% 2 M NH₃ in MeOH/CH₂Cl₂) afforded the title compound as a clear oil (65 mg, 82%). HPLC: Rₜ=4.73. MS (ESI): mass calcd. for C₃₀H₃₆ClN₅O₅S₂, 646.2; m/z found, 647.5 [M+H]⁺. ¹H NMR (CDCl₃): 7.80 (d, J=1.8, 1H), 7.61-7.57 (m, 2H), 7.51-7.44 (m, 2H), 7.38-7.34 (m, 2H), 4.98 (br t, J=6.0, 1H), 4.53 (s, 2H), 4.34 (d, J=6.1, 2H), 4.10 (t, J=6.8, 2H), 3.69 (t, J=4.5, 4H), 3.65 (t, J=5.8, 2H), 2.90 (s, 3H), 2.90-2.87 (m, 2H), 2.89 (s, 3H), 2.43-2.38 (m, 4H), 2.32 (t, J=6.8, 2H), 2.11-2.03 (m, 2H).

The compounds in Examples 71-72 were prepared using methods analogous to those described in Example 70, substituting the appropriate sulfonyl chloride or acid chloride for MsCl.

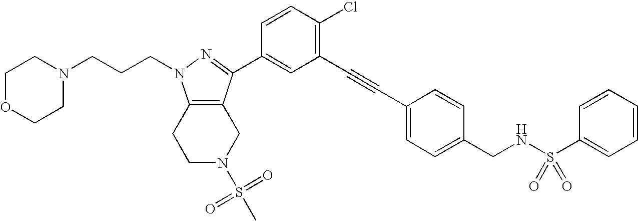

### Example 71

### N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzenesulfonamide

HPLC: Rₜ=5.19. MS (ESI): mass calcd. for C₃₅H₃₈ClN₅O₅S₂, 708.3; m/z found, 709.5 [M+H]⁺. ¹H NMR (CDCl₃): 7.88-7.84 (m, 1H), 7.78 (d, J=1.9, 1H), 7.62-7.57 (m, 1H), 7.54-7.45 (m, 6H), 7.19 (d, J=8.2, 2H), 5.10 (t, J=6.1, 1H), 4.52 (s, 2H), 4.16 (d, J=6.2, 2H), 4.10 (t, J=6.8, 2H), 3.68 (t, J=4.5, 4H), 3.64 (t, J=5.8, 2H), 2.90 (s, 3H), 2.90-2.87 (m, 2H), 2.42-2.37 (m, 4H), 2.32 (t, J=6.8, 2H), 2.10-2.03 (m, 2H).

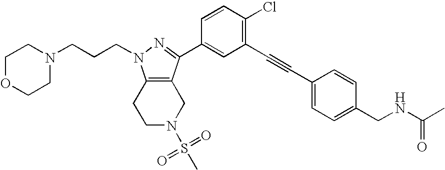

### Example 72

### N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-acetamide

MS (ESI): mass calcd. for C₃₁H₃₆ClN₅O₄S, 610.2; m/z found, 611.5 [M+H]⁺.

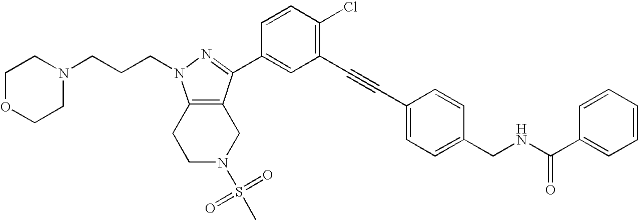

### Example 73

### N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzamide

HPLC: Rₜ=5.05. MS (ESI): mass calcd. for C₃₆H₃₈ClN₅O₄S, 672.3; m/z found, 673.5 [M+H]⁺. ¹H NMR (CDCl₃): 7.83-7.79 (m, 3H), 7.58-7.54 (m, 2H), 7.53-7.41 (m, 5H), 7.35 (d, J=8.2, 2H), 6.65 (br t, J=5.6, 1H), 4.66 (d, J=5.8, 2H), 4.52 (s, 2H), 4.09 (t, J=6.7, 2H), 3.68 (t, J=4.6, 4H), 3.64 (t, J=5.8, 2H), 2.89 (s, 3H), 2.89-2.86 (m, 2H), 2.42-2.37 (br m, 4H), 2.31 (t, J=6.8, 2H), 2.10-2.02 (m, 2H).

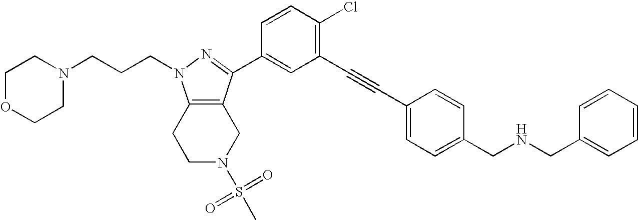

### Example 74

### Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine

A solution of 4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzylamine (87 mg, 0.153 mmol), benzaldehyde (17.2 μL, 0.17 mmol), and ACOH (9 μL, 0.153 mmol) in CH₂Cl₂ (1.5 mL) was stirred at rt for 30 min. The mixture was treated with NaB(OAc)₃H (42 mg, 0.20 mmol) and was stirred for 16 h. The reaction mixture was transferred directly to a silica gel column and purified (SiO₂; 0-5% 2 M NH₃ in MeOH/CH₂Cl₂) to afford the title compound as a clear oil (25 mg, 25%). HPLC: Rₜ=4.37. MS (ESI): mass calcd. for C₃₆H₄₀ClN₅O₃S, 658.3; m/z found, 659.5 [M+H]⁺. ¹H NMR (CDCl₃): 7.80 (d, J=1.8, 1H), 7.58-7.55 (m, 2H), 7.50-7.45 (m, 2H), 7.37-7.33 (m, 6H), 7.29-7.25 (m, 1H), 4.53 (s, 2H), 4.10 (t, J=6.8, 2H), 3.83 (s, 2H), 3.81 (s, 2H), 3.69 (t, J=4.5, 4H), 3.65 (t, J=5.7, 2H), 2.90 (s, 3H), 2.89 (t, J=5.7, 2H), 2.42-2.38 (br m, 4H), 2.32 (t, J=6.8, 2H), 2.11-2.03 (m, 2H), 1.73 (br s, 1H).

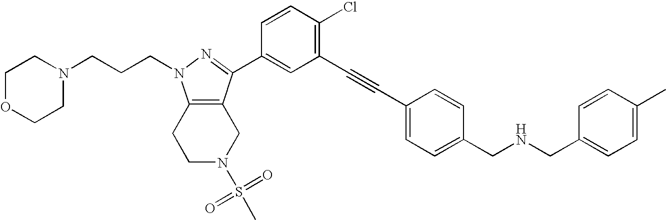

### Example 75

### (4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl]-benzyl)-(4-methyl-benzyl)-amine

A solution of 4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzaldehyde (50 mg, 0.088 mmol), 4-methylbenzylamine (13.4 μL, 0.106 mmol), and ACOH (6 μL, 0.106 mmol) in CH₂Cl₂ (1.0 mL) were stirred at rt for 30 min. The mixture was treated with NaB(OAc)₃H (24 mg, 0.106 mmol) and was stirred for 14 h. The reaction mixture was transferred directly to a silica gel column and purified (SiO₂; 0-5% 2 M NH₃ in MeOH/CH₂Cl₂) to afford the title compound as a clear oil (30 mg, 50%). MS (ESI): mass calcd. for C₃₇H₄₂ClN₅O₃S, 672.3; m/z found, 673.2 [M+H]⁺.

The compounds in Examples 76-79 were prepared using methods analogous to those described in Example 75, substituting the appropriate amine for 4-methylbenzylamine.

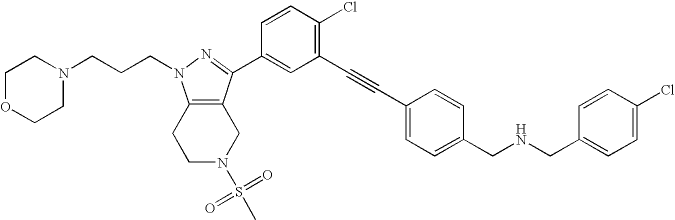

### Example 76

### (4-Chloro-benzyl)-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine

MS (ESI): mass calcd. for C₃₆H₃₉Cl₂N₅O₃S, 692.7; m/z found, 693.2 [M+H]⁺.

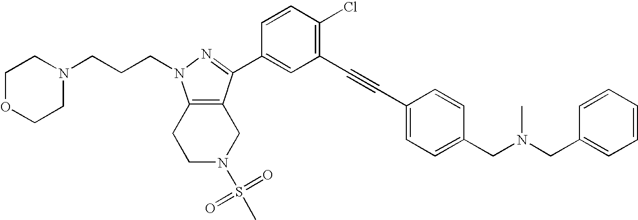

### Example 77

### Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methyl-amine

MS (ESI): mass calcd. for C₃₇H₄₂ClN₅O₃S, 672.3; m/z found, 673.2 [M+H]⁺.

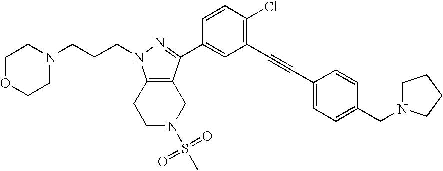

### Example 78

### 3-[4-Chloro-3-(4-pyrrolidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₃₃H₄₀ClN₅O₃S, 622.2; m/z found, 623.2 [M+H]⁺.

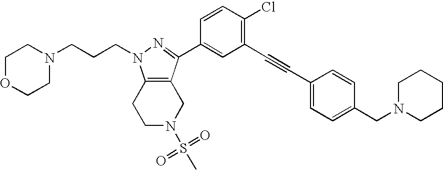

### Example 79

### 3-[4-Chloro-3-(4-piperidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine

MS (ESI): mass calcd. for C₃₄H₄₂ClN₅O₃S, 636.3; m/z found, 637.3 [M+H]⁺.

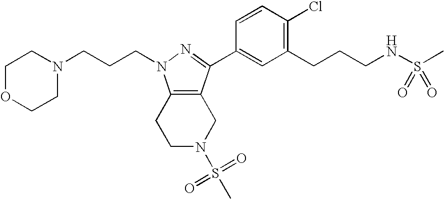

### Example 80

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-methanesulfonamide

A. 2-(3-[2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-isoindole-1,3-dione. This compound was prepared according to the methods described for Intermediate 1, substituting N-propargylphthalimide for TMSA in Step E. HPLC: Rₜ=4.48. MS (ESI): mass calcd. for C₃₁H₃₂ClN₅O₅S, 622.2; m/z found, 623.4 [M+H]⁺. ¹H NMR (CDCl₃): 7.90 (dd, J=5.4, 3.0, 2H), 7.75 (dd, J=5.5, 3.1, 2H), 7.68 (d, J=2.1, 1H), 7.45 (dd, J=8.5, 2.1, 1H), 7.37 (d, J=8.4, 1H), 4.75 (s, 2H), 4.48 (s, 2H), 4.07 (t, J=6.8, 2H), 3.68 (t, J=4.5, 4H), 3.63 (t, J=5.7, 2H), 2.90 (s, 3H), 2.87 (t, J=5.7, 2H), 2.42-2.37 (m, 4H), 2.30 (t, J=6.8, 2H), 2.08-2.01 (m, 2H).

B. 3-[2-Chloro-5-[5-methanesulfonyl-1-(3-morpholin-4-yl-propyl)-4,5,6,7-tetrahydro-1H-pyrazolo[4,3-c]pyridin-3-yl]-phenyl]-prop-2-ynylamine. To a solution of the above phthalimide (172 mg, 0.276 mmol) in 1:1 EtOH/DMF (2 mL) was added hydrazine monohydrate (134 μL). The reaction mixture was stirred at rt for 16 h, diluted with satd. aq. NaHCO₃, and extracted with CH₂Cl₂ (3×). The combined organic extracts were dried (Na₂SO₄), filtered, and concentrated to give a yellow oil. Purification (SiO₂; 0-10% 2 M NH₃ in MeOH/CH₂Cl₂) afforded the title compound as a white solid (86 mg, 63%). HPLC: Rₜ=3.84. MS (ESI): mass calcd. for C₂₃H₃₀ClN₅O₃S, 492.0; m/z found, 493.4 [M+H]⁺. ¹H NMR (CDCl₃): 7.67 (d, J=2.1, 1H), 7.49 (dd, J=8.4, 2.1, 1H), 7.41 (d, J=8.4, 1H), 4.52 (s, 2H), 4.09 (t, J=6.8, 2H), 3.73 (s, 2H), 3.70 (t, J=4.6, 4H), 3.65 (t, J=5.8, 2H), 2.90 (s, 3H), 2.89 (t, J=5.7, 2H), 2.43-2.38 (m, 4H), 2.32 (t, J=6.8, 2H), 2.11-2.04 (m, 2H), 1.58 (br s, 2H).

C. 3-[2-Chloro-5-[5-methanesulfonyl-1-(3-morpholin-4-yl-propyl)-4,5,6,7-tetrahydro-1H-pyrazolo[4,3-c]pyridin-3-yl]-phenyl]-propylamine. This compound was prepared from the above propynyl amine according to the methods described in Example 2. HPLC: Rₜ=3.97. MS (ESI): mass calcd. for C₂₃H₃₄ClN₅O₃S, 496.1; m/z found, 497.5 [M+H]⁺. ¹H NMR (CDCl₃): 7.54 (d, J=2.0, 1H), 7.36 (d, J=8.3, 1H), 7.30 (dd, J=8.3, 2.2, 1H), 4.50 (s, 2H), 4.09 (t, J=6.8, 2H), 3.70 (t, J=4.5, 4H), 3.65 (t, J=5.8, 2H), 3.28 (t, J=7.1, 2H), 2.90-2.80 (m, 4H), 2.88 (s, 3H), 2.43-2.38 (br m, 4H), 2.32 (t, J=6.8, 2H), 2.10-1.99 (m, 6H).

D. N-(3-[2-Chloro-5-[5-methanesulfonyl-1-(3-morpholin-4-yl-propyl)-4,5,6,7-tetrahydro-1H-pyrazolo[4,3-c]pyridin-3-yl-phenyl]-propyl)-methanesulfonamide.

To a solution of the above amine (50 mg, 0.101 mmol) and pyridine (20 μL) in CH₂Cl₂ (1.0 mL) was added MsCl (10 μL, 0.121 mmol). The reaction mixture was stirred at rt for 16 h. The mixture was concentrated and the crude residue transferred directly to a silica gel column. Purification (SiO₂; 0-3% 2 M NH₃ in MeOH/CH₂Cl₂) provided the title compound as a white foam (49 mg, 84%). HPLC: Rₜ=4.42. MS (ESI): mass calcd. for C₂₄H₃₆ClN₅O₅S₂, 574.2; m/z found, 575.4 [M+H]⁺. ¹H NMR (CDCl₃): 7.49 (s, 1H), 7.37 (d, J=8.3, 1H), 7.38-7.36 (m, 2H), 4.69 (t, J=6.1, 1H), 4.51 (s, 2H), 4.09 (t, J=6.9, 2H), 3.70 (t, J=4.6, 4H), 3.64 (t, J=5.8, 2H), 3.19 (q, J=6.7, 2H), 2.96 (s, 3H), 2.91 (s, 3H), 2.90-2.83 (m, 4H), 2.43-2.38 (br m, 4H), 2.32 (t, J=6.8, 2H), 2.10-2.03 (m, 2H), 1.98-1.91 (m, 2H).

The compounds in Examples 81-87 were prepared using methods analogous to those described in Example 80, Step D, substituting the appropriate sulfonyl chloride, acid chloride, or sulfamoyl chloride for MsCl.

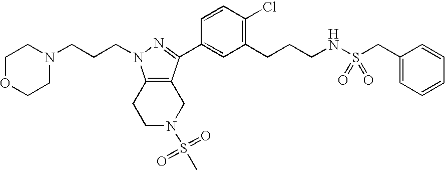

### Example 81

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-C-phenyl-methanesulfonamide

MS (ESI): mass calcd. for C₃₀H₄₀ClN₅O₅S₂, 650.3; m/z found, 651.5 [M+H]⁺.

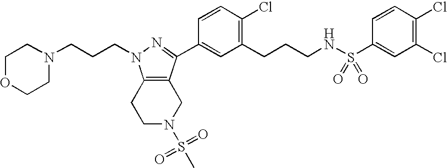

### Example 82

### 3,4-Dichloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide

MS (ESI): mass calcd. for C₂₉H₃₆Cl₃N₅O₅S₂, 705.1; m/z found, 706.4 [M+H]⁺.

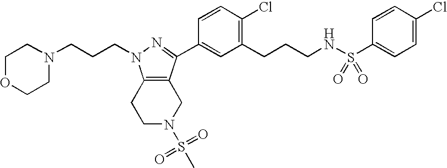

### Example 83

### 4-Chloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide

MS (ESI): mass calcd. for C₂₉H₃₆Cl₃N₅O₅S₂, 705.1; m/z found, 706.4 [M+H]⁺.

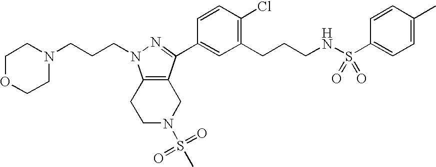

### Example 84

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methyl-benzenesulfonamide

MS (ESI): mass calcd. for C₃₀H₄₀ClN₅O₅S₂, 650.3; m/z found, 651.5 [M+H]⁺.

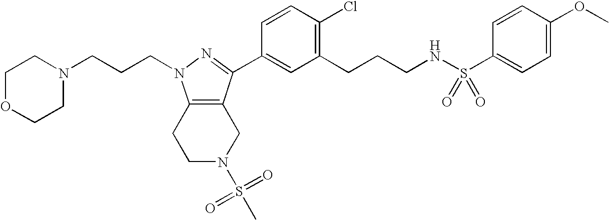

### Example 85

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methoxy-benzenesulfonamide

MS (ESI): mass calcd. for C₃₀H₄₀ClN₅O₆S₂, 666.3; m/z found, 667.5 [M+H]⁺.

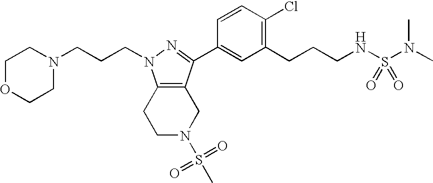

### Example 86

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl]-propyl)-N,N-dimethyl-sulfamoylurea

MS (ESI): mass calcd. for C₂₅H₃₉ClN₆O₅S₂, 603.2; m/z found, 604.4 [M+H]⁺.

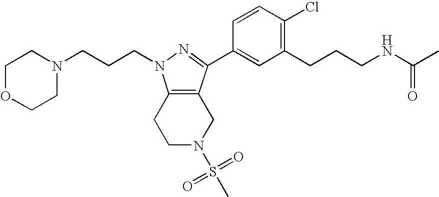

### Example 87

### N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-acetamide

MS (ESI): mass calcd. for C₂₅H₃₆ClN₅O₄S, 538.1; m/z found, 539.5 [M+H]⁺.

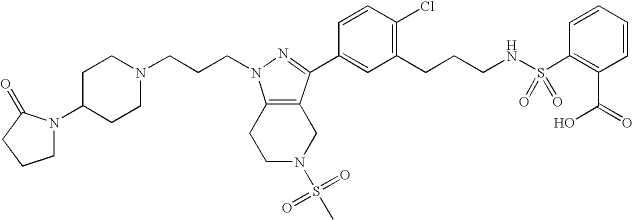

### Example 88

### 2-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propylsulfamoyl}-benzoic acid, trifluoroacetic acid salt

A. 1-(1-{3-[3-(4-Chloro-3-iodo-phenyl)-5-methanesulfonyl-4,5,6,7-tetrahydro-pyrazolo[4,3-c]pyridin-1-yl]-propyl}-piperidin-4-yl)-pyrrolidin-2-one. This compound was prepared according to the method described in Intermediate 1, Step D, substituting 1-piperidin-4-yl-pyrrolidin-2-one for morpholine. HPLC: Rₜ=4.65. MS (ESI): mass calcd. for C₂₅H₃₃CIIN₅O₃S, 646.0; m/z found, 647.3 [M+H]⁺. ¹H NMR (CDCl₃): 8.15 (s, 1H), 7.45 (m, 2H), 4.48 (s, 2H), 4.07 (t, J=6.8, 2H), 4.00-3.91 (m, 1H), 3.64 (t, J=5.7, 2H), 3.35 (t, J=7.0, 2H), 2.93-2.86 (m, 4H), 2.90 (s, 3H), 2.39 (t, J=8.1, 2H), 2.32 (t, J=6.9, 2H), 2.08-1.97 (m, 6H), 1.70-1.63 (m, 4H).

B. 2-[3-[2-Chloro-5-(5-methanesulfonyl-1f-3-[4-(2-oxo-pyrrolidin-1-yl)-piperidin-1-yl]-pro yl]-4,5,6,7-tetrahydro-1H-pyrazolo[4,3-c]pyridin-3-yl)-phenyl-prop-2-ynyl]-isoindole-1,3-dione. This compound was prepared from the above iodide according to the method described in Example 80, Step A. HPLC: Rₜ=4.79. MS (ESI): mass calcd. for C₃₆H₃₉ClN₆O₅S, 703.3; m/z found, 704.4 [M+H]⁺. ¹H NMR (CDCl₃): 7.90 (dd, J=5.4, 3.1, 2H), 7.75 (dd, J=5.4, 3.1, 2H), 7.68 (d, J=2.0, 1H), 7.45 (dd, J=8.4, 2.1, 1H), 7.37 (d, J=8.4), 4.75 (s, 2H), 4.48 (s, 2H), 4.06 (t, J=6.7, 2H), 4.00-3.91 (m, 1H), 3.64 (t, J=5.7, 2H), 3.35 (t, J=7.0, 2H), 2.93-2.85 (m, 4H), 2.91 (s, 3H), 2.38 (t, J=8.1, 2H), 2.32 (t, J=6.9, 2H), 2.08-1.96 (m, 6H), 1.70-1.62 (m, 4H).

C. 2-[3-[2-Chloro-5-(5-methanesulfonyl-1-[3-[4-(2-oxo-pyrrolidin-1-yl)-piperidin-1-yl]-propyl]-4,5,6,7-tetrahydro-1H-pyrazolo[4,3-c]pyridin-3-yl)-phenyl]-propyl]-isoindole-1,3-dione. This compound was prepared from the above alkyne according to the method described in Example 2. HPLC: Rₜ=4.82. MS (ESI): mass calcd. for C₃₆H₄₃ClN₆O₅S, 707.3; m/z found, 708.4 [M+H]⁺. ¹H NMR (CDCl₃): 7.84 (dd, J=5.4, 3.0, 2H), 7.72 (dd, J=5.4, 3.1, 2H), 7.57 (d, J=2.0, 1H), 7.35-7.33 (m, 2H), 4.54 (s, 2H), 4.08 (t, J=6.8, 2H), 4.00-3.91 (m, 1H), 3.79 (t, J=7.0, 2H), 3.65 (t, J=5.6, 2H), 3.35 (t, J=7.0, 2H), 2.93-2.86 (m, 4H), 2.92 (s, 3H), 2.83 (t, J=7.5, 2H), 2.38 (t, J=8.1, 2H), 2.33 (t, J=6.9, 2H), 2.10-1.97 (m, 8H), 1.70-1.62 (m, 4H).

D. 2-[3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]1-propylsulfamoyl}-benzoic acid methyl ester. To a solution of the above phthalimide (461 mg, 0.656 mmol) in EtOH (3.3 mL) was added hydrazine monohydrate (319 μL, 6.56 mmol). The reaction mixture was stirred at rt for 18 h. The resulting precipitate was filtered, and the filter cake washed with CH₂Cl₂. The filtrate was concentrated to provide a white solid, which was used directly in the next reaction without further purification.

To a solution of the crude amine (74 mg, 0.128 mmol) and pyridine (46 μL, 0.563 mmol) in CH₂Cl₂ (1.3 mL) was added 2-chlorosulfonyl-benzoic acid methyl ester (66 mg, 0.282 mmol). The reaction mixture was stirred at rt for 26 h and then transferred directly to a silica gel column. Purification (SiO₂; 0-5% 2 M NH₃ in MeOH/CH₂Cl₂) afforded the title compound as a clear oil (88 mg, 89%). HPLC: Rₜ=4.85. MS (ESI): mass calcd. for C₃₆H₄₇ClN₆O₇S₂, 775.4; m/z found, 776.3 [M+H]⁺. ¹H NMR (CDCl₃): 8.09-8.06 (m, 1H), 7.84-7.81 (m, 1H), 7.64-7.61 (m, 2H), 7.45 (s, 1H), 7.35-7.33 (m, 2H), 6.04 (t, J=6.1, 1H), 4.52 (s, 2H), 4.08 (t, J=6.7, 2H), 4.00-3.92 (m, 1H), 3.99 (s, 3H), 3.65 (t, J=5.6, 2H), 3.35 (t, J=7.0, 2H), 3.06 (q, J=6.5, 2H), 2.93-2.86 (m, 4H), 2.91 (s, 3H), 2.79 (t, J=7.5, 2H), 2.38 (t, J=8.1, 2H), 2.33 (t, J=6.9, 2H), 2.09-1.96 (m, 6H), 1.88-1.80 (m, 2H), 1.70-1.62 (m, 4H).

E. 2-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propylsulfamoyl}-benzoic acid, trifluoroacetic acid salt. To a solution of the above ester (63 mg, 0.078 mmol) in THF (0.8 mL) was added LiOH (2 M in H₂O; 0.2 mL). The reaction mixture was stirred at rt for 12 h, and then purified directly by reverse-phase HPLC (0.05% TFA/10-98% MeCN/H₂O) to provide the TFA salt of the title compound as a white solid (63 mg, 91%). HPLC: Rₜ=4.68. MS (ESI): mass calcd. for C₃₅H₄₅ClN₆O₇S₂, 761.4; m/z found, 762.2 [M+H]⁺. ¹H NMR (CD₃OD): 7.98-7.94 (m, 1H), 7.83-7.78 (m, 1H), 7.70-7.61 (m, 2H), 7.58 (s, 1H), 7.43-7.39 (m, 2H), 4.46 (s, 2H), 4.26 (t, J=6.0, 2H), 4.10-4.02 (m, 1H), 3.67-3.58 (br m, 2H), 3.35-3.24 (m, 4H), 3.17-2.95 (m, 4H), 2.97 (s, 3H), 2.92 (t, J=5.6, 2H), 2.81 (t, J=7.3, 2H), 2.34 (t, J=8.0, 2H), 2.33-2.29 (m, 2H), 2.01-1.94 (m, 4H), 1.90-1.78 (m, 6H).

The compounds in Examples 89-100 were prepared using methods analogous to those described in Example 88, Steps A-D, with the appropriate substituent changes.

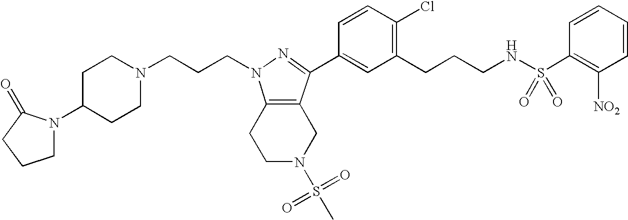

### Example 89

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-nitro-benzenesulfonamide

HPLC: Rₜ=4.82. MS (ESI): mass calcd. for C₃₄H₄₄ClN₇O₇S₂, 762.4; m/z found, 763.3 [M+H]⁺. ¹H NMR (CDCl₃): 8.13-8.10 (m, 1H), 7.85-7.82 (m, 1H), 7.74-7.71 (m, 2H), 7.45 (d, J=1.5, 1H), 7.35-7.33 (m, 2H), 5.54 (br s, 1H), 4.50 (s, 2H), 4.08 (t, J=6.8, 2H), 4.00-3.91 (m, 1H), 3.65 (t, J=5.7, 2H), 3.35 (t, J=7.0, 2H), 3.17 (t, J=6.4, 2H), 2.93-2.86 (m, 4H), 2.91 (s, 3H), 2.80 (t, J=7.5, 2H), 2.38 (t, J=8.1, 2H), 2.33 (t, J=6.9, 2H), 2.10-1.96 (m, 6H), 1.93-1.85 (m, 2H), 1.70-1.62 (m, 4H).

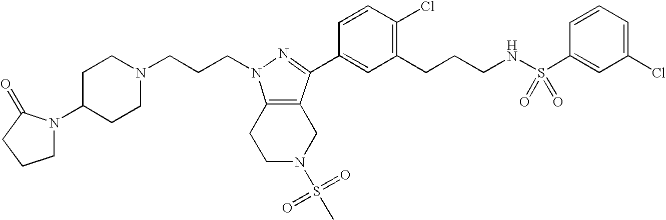

### Example 90

### 3-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide

HPLC: Rₜ=4.95. MS (ESI): mass calcd. for C₃₄H₄₄ClN₆O₅S₂, 751.8; m/z found, 753.3 [M+H]⁺. ¹H NMR (CDCl₃): 7.85 (t, J=1.8, 1H), 7.76-7.73 (m, 1H), 7.55-7.52 (m, 1H), 7.47-7.42 (m, 2H), 7.35-7.33 (m, 2H), 5.19 (br t, J=5.7, 1H), 4.51 (s, 2H), 4.07 (t, J=6.8, 2H), 4.00-3.91 (m, 1H), 3.65 (t, J=5.7, 2H), 3.35 (t, J=7.0, 2H), 3.17 (t, J=6.4, 2H), 3.00 (dd, J=12.8, 6.6, 4H), 2.94-2.86 (m, 4H), 2.91 (s, 3H), 2.79 (t, J=7.5, 2H), 2.39 (t, J=8.1, 2H), 2.34 (t, J=6.9, 2H), 2.10-1.96 (m, 6H), 1.89-1.85 (m, 2H), 1.70-1.62 (m, 4H).

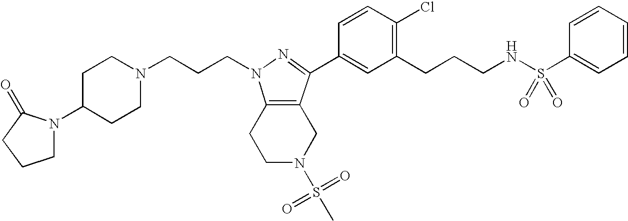

### Example 91

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide

MS (ESI): mass calcd. for C₃₄H₄₅ClN₆O₅S₂, 717.4; m/z found, 718.5 [M+H]⁺.

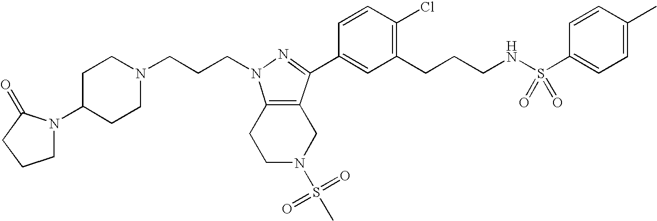

### Example 92

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-4-methyl-benzenesulfonamide

MS (ESI): mass calcd. for C₃₅H₄₇ClN₆O₅S₂, 731.4; m/z found, 732.5 [M+H]⁺.

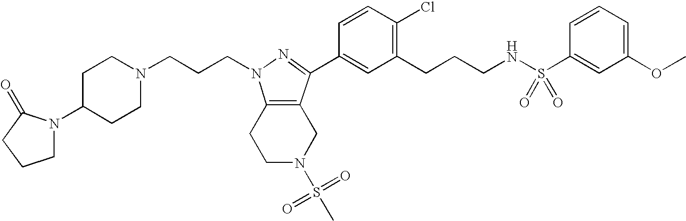

### Example 93

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methoxy-benzenesulfonamide

MS (ESI): mass calcd. for C₃₅H₄₇ClN₆O₆S₂, 747.4; m/z found, 748.3 [M+H]⁺.

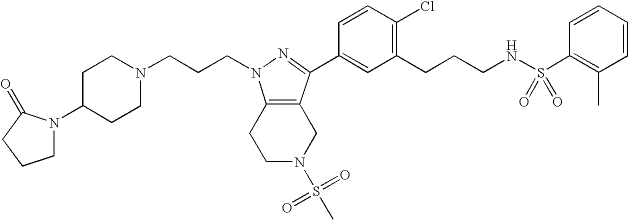

### Example 94

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methyl-benzenesulfonamide

MS (ESI): mass calcd. for C₃₅H₄₇ClN₆O₅S₂, 731.4; m/z found, 732.3 [M+H]⁺.

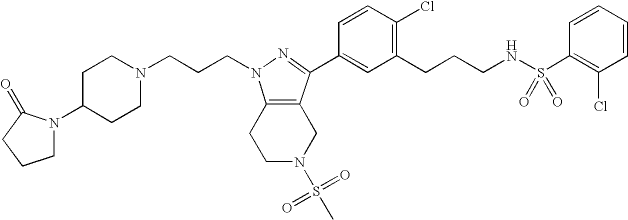

### Example 95

### 2-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide

MS (ESI): mass calcd. for C₃₄H₄₄Cl₂N₆O₅S₂, 751.8; m/z found, 753.2 [M+H]⁺.

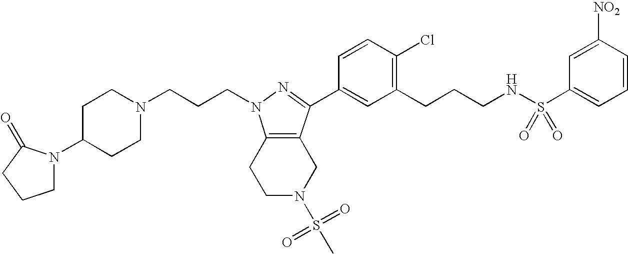

### Example 96

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-nitro-benzenesulfonamide

MS (ESI): mass calcd. for C₃₄H₄₄ClN₇O₇S₂, 762.4; m/z found, 763.2 [M+H]⁺.

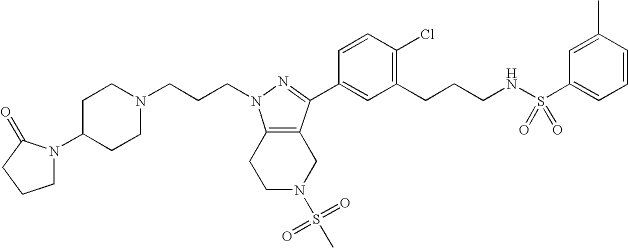

### Example 97

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methyl-benzenesulfonamide

MS (ESI): mass calcd. for C₃₅H₄₇ClN₆O₅S₂, 731.4; m/z found, 732.3 [M+H]⁺.

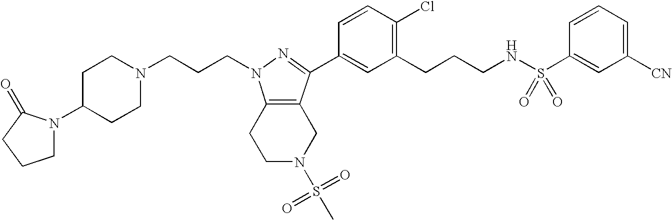

### Example 98

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-cyano-benzenesulfonamide

MS (ESI): mass calcd. for C₃₅H₄₄ClN₇O₅S₂, 742.4; m/z found, 743.3 [M+H]⁺.

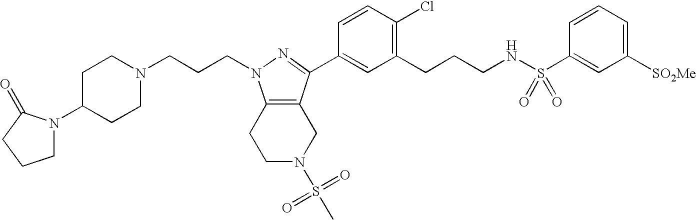

### Example 99

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methanesulfonyl-benzenesulfonamide

MS (ESI): mass calcd. for C₃₅H₄₇ClN₆O₇S₃, 795.5; m/z found, 796.2 [M+H]⁺.

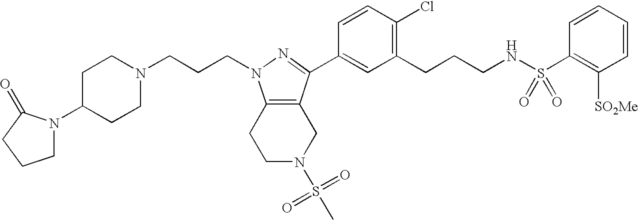

### Example 100

### N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methanesulfonyl-benzenesulfonamide

MS (ESI): mass calcd. for C₃₅H₄₇ClN₆O₇S₃, 795.5; m/z found, 796.3 [M+H]⁺.

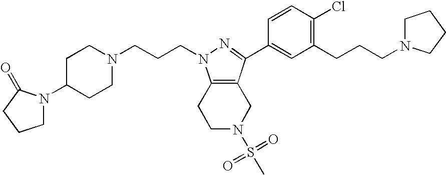

### Example 101

### 1-[1-(3-{3-[4-Chloro-3-(3-pyrrolidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one

A. 1-[1-(3-[3-{4-Chloro-3-(3-hydroxy-prop-1-ynyl)-phenyl]-5-methanesulfonyl-4,5,6,7-tetrahydro-pyrazolo[4,3-c]pyridin-1-yl}-propyl)-piperidin-4-yl]-pyrrolidin-2-one. This compound was prepared following the methods described in Example 80, Step A, substituting propargyl alcohol for N-propargylphthalimide. HPLC Rₜ=4.30. MS (ESI): mass calcd. for C₂₈H₃₆ClN₅O₄S, 574.2; m/z found, 575.3 [M+H]⁺. ¹H NMR (CDCl₃): 7.64 (d, J=2.1, 1H), 7.48 (dd, J=8.4, 2.1, 1H), 7.39 (d, J=8.4, 1H), 4.54 (s, 2H), 4.46 (s, 2H), 4.05 (t, J=6.8, 2H), 3.99-3.91 (m, 1H), 3.61 (t, J=5.7, 2H), 3.35 (t, J=7.0, 2H), 2.94 (br s, 1H), 2.91 (s, 3H), 2.89-2.83 (m, 4H), 2.39 (t, J=7.9, 2H), 2.34 (t, J=7.2, 2H), 2.10-1.97 (m, 6H), 1.71-1.64 (m, 4H).

B. 1-fi-(3-[3-{4-Chloro-3-(3-hydroxy-propyl)-phenyl]-5-methanesulfonyl-4,5,6,7-tetrahydro-pyrazolo[4,3-c]pyridin-4-yl-pyrrolidin-2-one. This compound was prepared from the above alkyne following the methods described in Example 2. MS (ESI): mass calcd. for C₂₈H₄₀ClN₅O₄S, 578.2; m/z found, 579.3.

C. 1-fi-(3-[3-[4-Chloro-3-(3-pyrrolidin-1-yl-propyl)-phenyl]-5-methanesulfonyl-4,5,6,7-tetrahydro-pyrazolo[4,3-c]pyridin-1-yl]-propyl)-piperidin-4-yl]-pyrrolidin-2-one. To a solution of the above alcohol (584 mg, 1.01 mmol) and pyridine (250 μL, 3.03 mmol) in CH₂Cl₂ (8 mL) was added MsCl (120 μL, 1.52 mmol). The reaction mixture was stirred at rt for 4 h, diluted with H₂O, and extracted with CH₂Cl₂ (3×). The combined organic extracts were dried (Na₂SO₄), filtered, and concentrated to give a tan solid, which was used directly without further purification. To a solution of the crude mesylate (100 mg, 0.152 mmol) in EtOH (1.0 mL) was added pyrrolidine (3.04 mmol). The reaction mixture was heated to 60° C. and stirred for 16 h. The solvent was removed and the crude residue was purified (SiO₂; 2 M NH₃ in MeOH/CH₂Cl₂) to provide the title compound as a white solid (54 mg, 71%). HPLC: Rₜ=4.01. MS (ESI): mass calcd. for C₃₂H₄₇ClN₆O₃S, 631.3; m/z found, 632.3 [M+H]⁺. ¹H NMR (CDCl₃): 7.52 (d, J=2.0, 1H), 7.36 (d, J=8.2, 1H), 7.29 (dd, J=8.3, 2.1, 1H), 4.50 (s, 2H), 4.08 (t, J=6.8, 2H), 4.01-3.93 (m, 1H), 3.65 (t, J=5.7, 2H), 3.35 (t, J=7.0, 2H), 2.93-2.86 (m, 4H), 2.89 (s, 3H), 2.83-2.78 (m, 2H), 2.55-2.49 (m, 6H), 2.39 (t, J=7.9, 2H), 2.33 (t, J=7.0, 2H), 2.09-2.03 (m, 8H), 2.09-1.97 (m, 6H), 1.92-1.85 (m, 2H), 1.80-1.76 (m, 4H), 1.70-1.63 (m, 4H).

The compounds in Examples 102-108 were prepared using methods analogous to those described in Example 101 with the appropriate substituent changes.

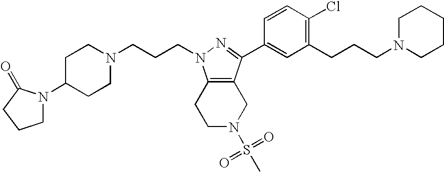

### Example 102

### 1-[1-(3-{3-[4-Chloro-3-(3-piperidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₃H₄₉ClN₆O₃S, 645.3; m/z found, 646.3 [M+H]⁺.

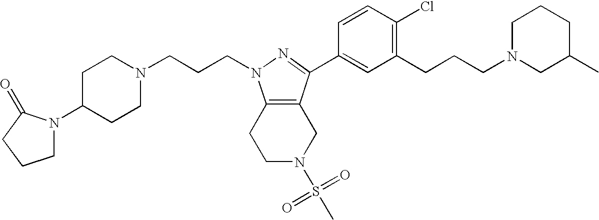

### Example 103

### 1-{1-[3-(3-{4-Chloro-3-[3-(3-methyl-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₅₁ClN₆O₃S, 659.3; m/z found, 660.4 [M+H]⁺.

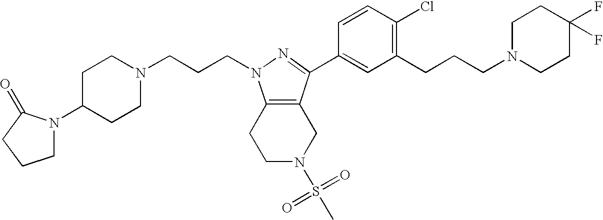

### Example 104

### 1-{1-[3-(3-{4-Chloro-3-[3-(4,4-difluoro-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₄₇ClF₂N₆O₃S, 681.3; m/z found, 682.4 [M+H]⁺.

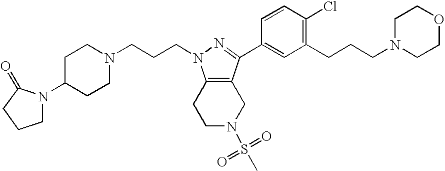

### Example 105

### 1-[1-(3-{3-[4-Chloro-3-(3-morpholin-4-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₂H₄₇ClN₆O₄S, 647.3; m/z found, 648.3 [M+H]⁺.

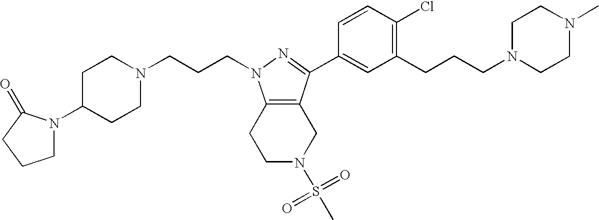

### Example 106

### 1-{1-[3-(3-{4-Chloro-3-[3-(4-methyl-piperazin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₃H₅₀ClN₇O₃S, 660.3; m/z found, 661.4 [M+H]⁺.

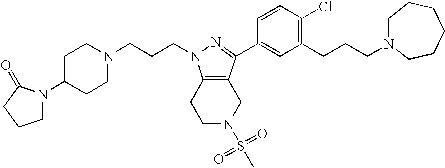

### Example 107

### 1-[1-(3-{3-[3-(3-Azepan-1-yl-propyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₄H₅₁ClN₆O₃S, 659.3; m/z found, 660.3 [M+H]⁺.

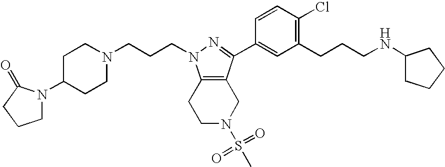

### Example 108

### 1-[1-(3-{3-[4-Chloro-3-(3-cyclopentylamino-propyl)-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₃H₄₉ClN₆O₃S, 645.3; m/z found, 646.3 [M+H]⁺.

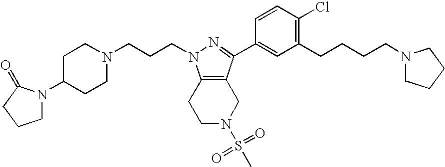

### Example 109

### 1-[1-(3-{3-[4-Chloro-3-(4-pyrrolidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one

A. 1-[1-(3-[3-{4-Chloro-3-(4-hydroxy-but-1-ynyl)-phenyl]-5-methanesulfonyl-4,5,6,7-tetrahydro-pyrazolo[4,3-c]pyridin-1-yl}-prolyl)-piperidin-4-yl]-pyrrolidin-2-one. This compound was prepared following the methods described in Example 101, Step A, substituting for 3-butyn-1-ol for propargyl alcohol. HPLC: Rₜ=4.33. MS (ESI): mass calcd. for C₂₉H₃₈ClN₅O₄S, 588.2; m/z found, 589.3 [M+H]⁺. ¹H NMR (CDCl₃): 7.68 (d, J=1.3, 1H), 7.45 (dd, J=8.4, 1.6, 1H), 7.40 (d, J=8.4, 1H), 4.49 (s, 2H), 4.06 (t, J=6.6, 2H), 3.99-3.91 (m, 1H), 3.85 (t, J=6.2, 1H), 3.63 (t, J=5.7, 2H), 3.35 (t, J=7.0, 2H), 2.93-2.84 (m, 4H), 2.90 (s, 3H), 2.76 (t, J=6.3, 2H), 2.39 (t, J=7.9, 2H), 2.33 (t, J=7.2, 2H), 2.09-1.97 (m, 6H), 1.70-1.62 (m, 4H).

B. 1-[1-(3-{3-[4-Chloro-3-(4-hydroxy-butyl)-phenyl]-5-methanesulfonyl-4,5,6,7-tetrahydro-pyrazolo[43-c]pyridin-4-yl-pyrrolidin-2-one. This compound was prepared from the above alkyne following the methods described in Example 101, Step B. MS (ESI): mass calcd. for C₂₉H₄₂ClN₅O₄S, 592.2; m/z found, 593.4 [M+H]

C. 1-[1-(3-{3-[4-Chloro-3-(4-pyrrolidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one. The title compound was prepared from the above alcohol following the methods described in Example 101, Step C. HPLC: Rₜ=4.07. MS (ESI): mass calcd. for C₃₃H₄₉ClN₆O₃S, 645.3; m/z found, 646.4 [M+H]⁺. ¹H NMR (CDCl₃): 7.50 (d, J=2.0, 1H), 7.35 (d, J=8.2, 1H), 7.28 (dd, J=8.3, 2.1, 1H), 4.50 (s, 2H), 4.08 (t, J=6.8, 2H), 4.01-3.92 (m, 1H), 3.65 (t, J=5.7, 2H), 3.35 (t, J=7.0, 2H), 2.92-2.87 (m, 4H), 2.89 (s, 3H), 2.78 (t, J=7.3, 2H), 2.54-2.47 (m, 6H), 2.39 (t, J=7.9, 2H), 2.33 (t, J=7.0, 2H), 2.09-1.98 (m, 6H), 1.80-1.75 (m, 4H), 1.71-1.60 (m, 8H).

The compounds in Examples 110-115 were prepared using methods analogous to those described in Example 109 with the appropriate substituent changes.

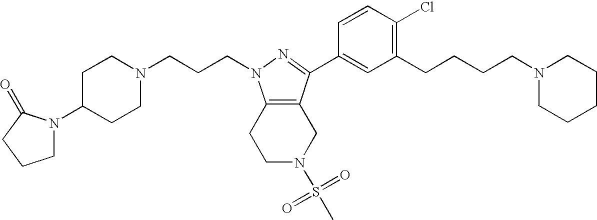

### Example 110

### 1-[1-(3-{3-[4-Chloro-3-(4-piperidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₄H₅₁ClN₆O₃S, 659.3; m/z found, 660.4 [M+H]⁺.

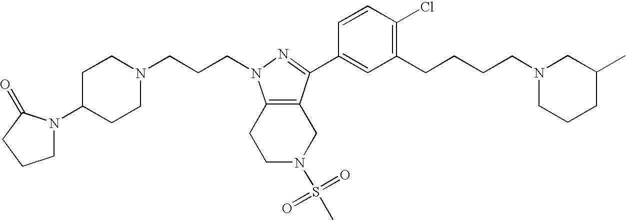

### Example 111

### 1-{1-[3-(3-{4-Chloro-3-[4-(3-methyl-piperidin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₅H₅₃ClN₆O₃S, 673.4; m/z found, 674.4 [M+H]⁺.

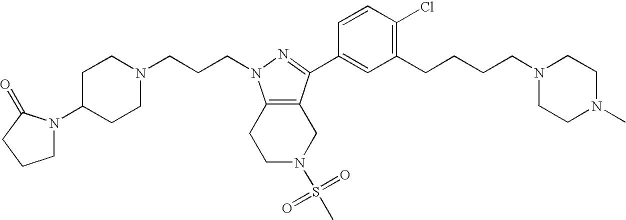

### Example 112

### 1-{1-[3-(3-{4-Chloro-3-[4-(4-methyl-piperazin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one

MS (ESI): mass calcd. for C₃₄H₅₂ClN₇O₃S, 674.4; m/z found, 675.4 [M+H]⁺.

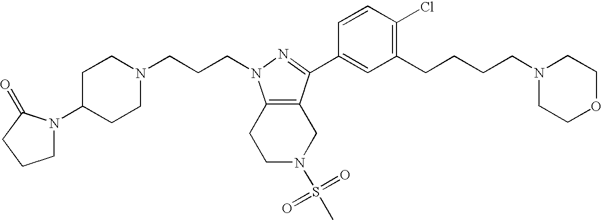

### Example 113

### 1-[1-(3-{3-[4-Chloro-3-(4-morpholin-4-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₃H₄₉ClN₆O₄S, 661.3; m/z found, 662.4 [M+H]⁺.

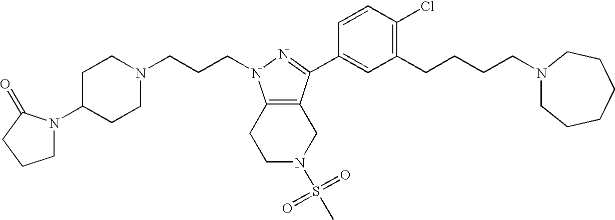

### Example 114

### 1-[1-(3-{3-[3-(4-Azepan-1-yl-butyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₅H₅₃ClN₆O₃S, 673.4; m/z found, 674.5 [M+H]⁺.

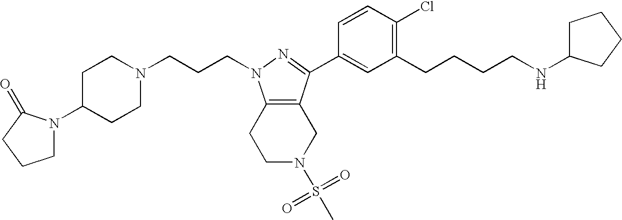

### Example 115

### 1-[1-(3-{3-[4-Chloro-3-(4-cyclopentylamino-butyl)-phenyl]-5-methanesulfonyl-4,5,6,7-tetrahydro-pyrazolo[4,3-c]pyridin-1-yl}-propyl)-piperidin-4-yl]-pyrrolidin-2-one

MS (ESI): mass calcd. for C₃₄H₅₁ClN₆O₃S, 659.3; m/z found, 660.4 [M+H]⁺.

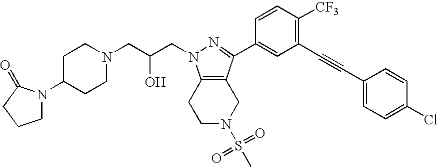

### Example 116

### 1-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one

A. 3-Amino-4-trifluoromethyl-benzoic acid. A mixture of 3-nitro-4-trifluoromethylbenzoic acid (19.5 g, 83 mmol) and 10% Pd/C (4 g) in EtOAc (300 mL) was shaken under 40 psi of H₂ in a pressure vessel. The vessel was repeatedly refilled until the pressure stabilized at 40 psi. Shaking was continued for 10 min, and then N₂ was bubbled through the mixture for 30 min. The resulting black mixture was filtered through diatomaceous earth and washed with MeOH. The filtrate was concentrated to afford the desired product as a white solid (19.5 g, 98%), which needed no further purification. HPLC: Rₜ=5.46. MS (ESI): mass calcd. for C₈H₆F₃NO₂, 205.1; m/z found, 204.3 [M−H]⁻. ¹H NMR (CD₃OD): 7.51 (s, 1H), 7.45 (d, J=8.2, 1H), 7.30 (d, J=8.0, 1H), 5.02 (br s, 3H).

B. 3-Iodo-4-trifluoromethyl-benzoic acid. To a −5 LC slurry of the above aniline (16.6 g, 80.9 mmol) and conc. H₂SO₄ (33 mL) in H₂O (17 mL) was added a solution of NaNO₂ (6.7 g, 97.1 mmol) in H₂O (25 mL). The mixture was stirred at 0 LC for 1 h, then treated with a solution of KI (26.9 g, 161.8 mmol) and 12 (1 crystal) in H₂O (30 mL). The reaction mixture was stirred for 1 h at rt and then heated at reflux for 4 h, during which time significant foaming and frothing was observed. The thick brown mixture was cooled to 0° C., diluted with satd. aq. Na₂S₂O₃ (100 mL), and filtered to provide a beige solid. The solid was washed with H₂O and recrystallized from CH₃CN to afford the desired product as a white solid (18.3 g, 72%). HPLC: Rₜ=6.33. MS (ESI): mass calcd. for C₈H₄F₃IO₂, 316.0; m/z found, 315.2 [M−H]⁻. ¹H NMR (CD₃OD): 8.68 (s, 1H), 8.11 (d, J=7.9, 1H), 7.73 (d, J=8.2, 1H), 4.44 (br s, 1H).

C. 3-(3-Iodo-4-trifluoromethyl-phenyl)-5-methanesulfonyl-4,5,6,7-tetrahydro-1H-pyrazolo[4,3-c]pyridine. To a 0 LC solution of the above acid (6.3 g, 20 mmol) and oxalyl chloride (1.9 mL, 22 mmol) in benzene (20 mL) was added DMF (0.15 mL). The reaction mixture was warmed to rt and stirred for 5 h. The mixture was concentrated to afford a white solid, which was used directly in the next reaction following the procedure outlined in Intermediate 1, Step B. HPLC: Rₜ=6.25. MS (ESI): mass calcd. for C₁₄H₁₃F₃1N₃O₂S, 471.2; m/z found, 472.3 [M+H]⁺. ¹H NMR (DMSO-d₆): 8.39 (s, 1H), 7.82 (d, J=8.3, 1H), 7.72 (d, J=8.3, 1H), 4.49 (s, 2H), 3.52 (t, J=5.7, 2H), 3.00 (s, 3H), 2.85 (t, J=5.6, 2H).

D. 1-[1-(3-[3-[3-(4-Chloro-phenylethynyl)-4-trifluoromethyl-phenyl]-5-methanesulfonyl-4,5,6,7-tetrahydro-pyrazolo[4,3-c]pyridin-1-yl]-2-hydroxy-propyl)-piperidin-4-yl]-pyrrolidin-2-one. To a slurry of the above pyrazole (1.28 g, 2.72 mmol) and Cs₂CO₃ (1.33 g, 4.08 mmol) in DMF (9 mL) was added epichlorohydrin (1.26 g, 13.6 mmol). The reaction mixture was stirred at rt for 17 h, then cooled to 0 LC, and treated slowly with ice water. The resulting precipitate was filtered to provide the desired epoxide as a white solid, which was used directly in the next reaction. The crude epoxide (105 mg, 0.199 mmol) suspended in EtOH (1.0 mL) and treated with 1-piperidin-4-yl-pyrrolidin-2-one (37 mg, 0.219 mmol). The reaction mixture was heated at reflux for 16 h and then concentrated. Purification (SiO₂; 0-3% 2 M NH₃ in MeOH/CH₂Cl₂) provided a white solid, which was converted to the title compound following the procedure described in Intermediate 1, Step E, substituting 4-chloro-ethynylbenzene for TMSA. HPLC: Rₜ=5.45. MS (ESI): mass calcd. for C₃₄H₃₇ClF₃N₅O₄S, 704.2; m/z found, 705.4 [M+H]⁺. ¹H NMR (CDCl₃): 7.91 (s, 1H), 7.72 (d, 8.3, 1H), 7.61 (d, J=8.3, 1H), 7.50 (d, J=8.5, 2H), 7.35 (d, J=8.6, 2H), 4.58 (dd, J=18.4, 14.6, 2H), 4.19 (dd, J=13.8, 2.8, 1H), 4.15-4.09 (br m, 1H), 4.02-3.94 (br m, 2H), 3.73-3.62 (m, 2H), 3.34 (t, J=7.0, 2H), 3.09-2.99 (m, 2H), 2.95-2.92 (m, 2H), 2.89 (s, 3H), 2.49-2.34 (m, 5H), 2.16-2.09 (m, 1H), 2.04-1.96 (m, 2H), 1.78-1.61 (m, 4H).

The compounds in Examples 117-122 were prepared using methods analogous to those described in Example 116, with the appropriate substituent changes.

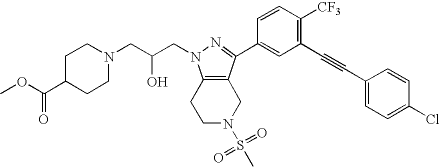

### Example 117

### 1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid methyl ester

HPLC: Rₜ=5.73. MS (ESI): mass calcd. for C₃₂H₃₄ClF₃N₄O₅S, 679.2; m/z found, 680.4 [M+H]⁺. ¹H NMR (CDCl₃): 7.91 (s, 1H), 7.71 (d, J=8.3, 1H), 7.61 (d, J=8.2, 1H), 7.50 (d, J=8.6, 2H), 7.35 (d, J=8.6, 2H), 4.58 (dd, J=22.5, 14.4, 2H), 4.20 (dd, J=13.7, 2.9, 1H), 4.17-4.14 (m, 1H), 4.01 (dd, J=13.6, 6.5, 1H), 3.75-3.61 (m, 2H), 3.68 (s, 3H), 3.11-3.04 (m, 1H), 2.96-2.93 (m, 2H), 2.89 (s, 3H), 2.49-2.31 (m, 4H), 2.15-2.10 (m, 1H), 1.94-1.90 (m, 2H), 1.83-1.66 (m, 4H).

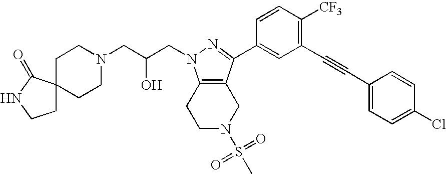

### Example 118; 8-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-2,8-diaza-spiro[4.5]decan-1-one.

HPLC: Rₜ=5.43. MS (ESI): mass calcd. for C₃₃H₃₅ClF₃N₅O₄S, 690.2; m/z found, 691.4 [M+H]⁺. ¹H NMR (CDCl₃): 7.91 (s, 1H), 7.71 (d, J=8.3, 1H), 7.60 (d, J=8.3, 1H), 7.50 (d, J=8.6, 2H), 7.35 (d, J=8.6, 2H), 6.37 (s, 1H), 4.58 (dd, J=21.4, 14.5, 2H), 4.20 (dd, J=13.7, 2.8, 1H), 4.16-4.12 (br m, 1H), 4.02 (dd, J=13.6, 6.6, 1H), 3.75-3.61 (m, 2H), 3.32 (t, J=6.9, 2H), 3.11-3.04 (m, 1H), 2.96-2.93 (m, 2H), 2.89 (s, 3H), 2.83-2.80 (br m, 1H), 2.50-2.37 (m, 3H), 2.18-2.12 (m, 1H), 2.02 (t, J=6.9, 2H), 2.01-1.85 (m, 3H), 1.46 (br m, 2H).

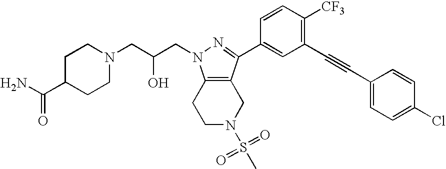

### Example 119

### 1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid amide

HPLC: Rₜ=5.31. MS (ESI): mass calcd. for C₃₁H₃₃ClF₃N₅O₄S, 664.2; m/z found, 665.4 [M+H]⁺. ¹H NMR (CDCl₃): 7.91 (s, 1H), 7.71 (d, J=8.3, 1H), 7.60 (d, J=8.3, 1H), 7.50 (d, J=8.5, 2H), 7.35 (d, J=8.6, 2H), 6.58 (br s, 1H), 6.49 (br s, 1H), 4.57 (dd, J=19.1, 14.5, 2H), 4.19 (dd, J=13.8, 2.8, 1H), 4.16-4.09 (br m, 1H), 4.00 (dd, J=13.8, 6.7, 1H), 3.73-3.61 (m, 2H), 3.14-3.03 (m, 1H), 2.99-2.84 (m, 2H), 2.89 (s, 3H), 2.45-2.35 (m, 2H), 2.32-2.24 (m, 1H), 2.20-2.12 (m, 1H), 2.06-1.98 (m, 1H), 1.86-1.63 (m, 4H).

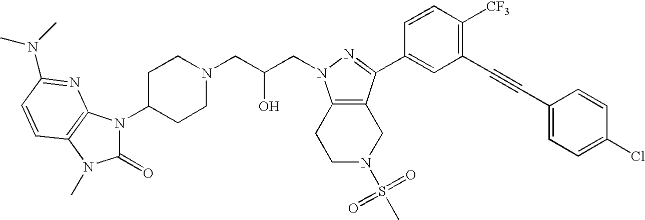

### Example 120

### 3-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-1,3-dihydro-imidazo[4,5-b]pyridin-2-one

HPLC: Rₜ=5.69. MS (ESI): mass calcd. for C₃₉H₄₂ClF₃N₈O₄S, 811.3; m/z found, 812.5 [M+H]⁺. ¹H NMR (CDCl₃): 7.92 (s, 1H), 7.71 (d, J=8.3, 1H), 7.62 (d, J=7.7, 1H), 7.50 (dt, J=2.1, 8.6, 2H), 7.35 (dt, J=2.1, 8.6, 2H), 7.03 (d, J=8.5, 1H), 6.16 (d, J=8.5, 1H), 4.59 (dd, J=23.5, 15.0, 2H), 4.41-4.33 (m, 1H), 4.23 (dd, J=13.8, 2.7, 1H), 4.19-4.13 (br m, 1H), 4.05 (dd, J=13.8, 6.8, 1H), 3.77-3.63 (m, 2H), 3.34 (s, 3H), 3.16-3.05 (m, 1H), 3.03 (s, 6H), 2.98-2.93 (m, 2H), 2.90 (s, 3H), 2.86-2.70 (m, 2H), 2.57-2.44 (m, 3H), 2.26-2.20 (m, 1H), 1.77-1.74 (br m, 2H).

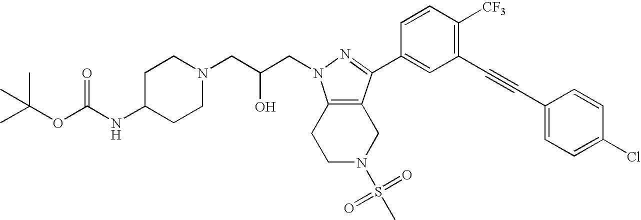

### Example 121

### [1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester

HPLC: Rₜ=5.96. MS (ESI): mass calcd. for C₃₅H₄₁ClF₃N₅O₅S, 736.3; m/z found, 737.5 [M+H]⁺. ¹H NMR (CDCl₃): 7.91 (s, 1H), 7.71 (d, J=8.3, 1H), 7.60 (d, J=8.8, 1H), 7.52-7.48 (m, 2H), 7.38-7.33 (m, 2H), 4.57 (dd, J=20.5, 14.5, 2H), 4.47 (br s, 1H), 4.18 (dd, J=13.8, 2.8, 1H), 4.15-4.07 (br m, 1H), 3.99 (dd, J=13.8, 6.8, 1H), 3.73-3.60 (m, 2H), 3.47 (br s, 1H), 3.09-3.02 (m, 1H), 2.96-2.86 (m, 3H), 2.88 (s, 3H), 2.77-2.70 (br m, 1H), 2.47-2.34 (m, 3H), 2.12 (t, J=11.1, 1H), 1.93 (br d, J=11.1, 2H), 1.49-1.36 (m, 2H), 1.44 (s, 9H).

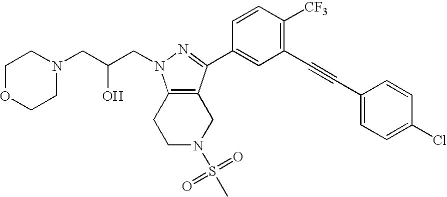

### Example 122

### 1-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-3-morpholin-4-yl-propan-2-ol

MS (ESI): mass calcd. for C₂₉H₃₀ClF₃N₄O₄S, 623.1; m/z found, 624.4 [M+H]⁺.

The compounds in Examples 123-131 were prepared from 3-(4-chloro-3-iodo-phenyl)-5-methanesulfonyl-4,5,6,7-tetrahydro-1H-pyrazolo[4,3-c]pyridine (Intermediate 1, Step B) according to the methods described in Example 116, Step D, substituting the appropriate amine for 1-piperidin-4-yl-pyrrolidin-2-one.

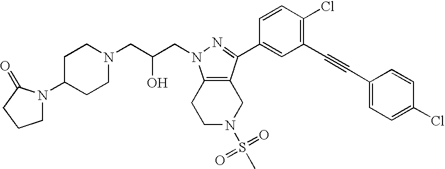

### Example 123

### 2-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-cyclopentanone

HPLC: Rₜ=5.29. MS (ESI): mass calcd. for C₃₃H₃₇Cl₂N₅O₄S, 670.7; m/z found, 671.5 [M+H]⁺. ¹H NMR (CDCl₃): 7.79 (d, J=1.7, 1H), 7.54-7.47 (m, 4H), 7.35 (dd, J=6.6, 1.9, 2H), 4.55 (d, J=4.3, 2H), 4.17 (dd, J=13.8, 2.8, 1H), 4.13-4.07 (m, 1H), 4.02-3.96 (m, 2H), 3.73-3.62 (m, 3H), 3.34 (t, J=7.0, 2H), 3.08-2.99 (m, 2H), 2.94-2.91 (m, 3H), 2.85 (s, 3H), 2.45-2.37 (m, 5H), 2.00 (t, J=7.6, 2H), 1.75-1.61 (m, 4H).

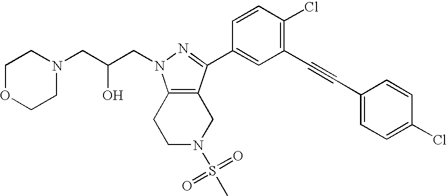

### Example 124

### 1-{3-[4-Chloro-3-(4-chloro-phenylethynyl)-phenyl]-5-methanesulfonyl-4,5,6,7-tetrahydro-pyrazolo[4,3-c]pyridin-1-yl}-3-morpholin-4-yl-propan-2-ol

HPLC: Rₜ=5.47. MS (ESI): mass calcd. for C₂₈H₃₀Cl₂N₄O₄S, 589.6; m/z found, 590.2 [M+H]⁺. ¹H NMR (CDCl₃): 7.78 (d, J=1.5, 1H), 7.54-7.50 (m, 2H), 7.48-7.45 (m, 2H), 7.37-7.32 (m, 2H), 4.53 (dd, J=18.3, 14.5, 2H), 4.18 (dd, J=13.3, 2.9, 1H), 4.17-4.10 (m, 1H), 4.02-3.96 (m, 1H), 3.74-3.59 (m, 7H), 3.07-2.99 (m, 1H), 2.94-2.88 (m, 1H), 2.87 (s, 3H), 2.65-2.58 (m, 2H), 2.48-2.38 (m, 4H).

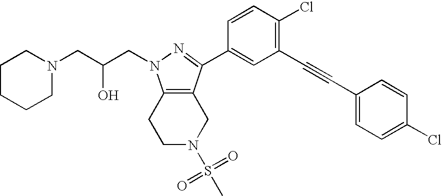

### Example 125

### 1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperidin-1-yl-propan-2-ol

HPLC: Rₜ=5.64. MS (ESI): mass calcd. for C₂₉H₃₂Cl₂N₄O₃S, 587.6; m/z found, 588.5 [M+H]⁺. ¹H NMR (CDCl₃): 7.79 (d, J=1.9, 1H), 7.53 (d, J=8.5, 2H), 7.49-7.47 (m, 2H), 7.35 (d, J=8.5, 2H), 4.55 (dd, J=24.4, 14.5, 2H), 4.16 (dd, J=13.7, 2.8, 1H), 4.13-4.08 (m, 1H), 3.98 (dd, J=13.7, 6.8, 1H), 3.73-3.60 (m, 2H), 3.12-3.05 (m, 1H), 2.96-2.90 (m, 1H), 2.88 (s, 3H), 2.56 (br m, 2H), 2.42-2.27 (m, 4H), 1.59-1.52 (m, 4H), 1.47-1.42 (m, 3H).

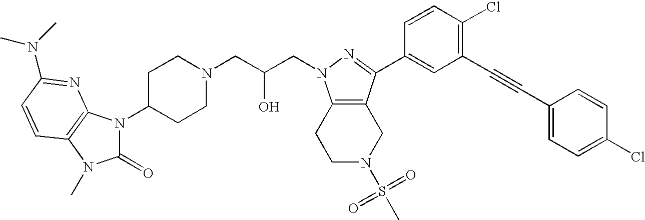

### Example 126

### 3-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-1,3-dihydro-imidazo[4,5-b]pyridin-2-one

HPLC: Rₜ=5.60. MS (ESI): mass calcd. for C₃₈H₄₂Cl₂N₈O₄S, 777.8; m/z found, 778.4 [M+H]⁺. ¹H NMR (CDCl₃): 7.80 (d, J=1.9, 1H), 7.53-7.45 (m, 4H), 7.34 (dd, J=6.6, 2.0, 2H), 7.03 (d, J=8.5, 1H), 6.16 (d, J=8.5, 1H), 4.56 (dd, J=23.7, 14.5, 2H), 4.40-4.32 (m, 1H), 4.21 (dd, J=13.8, 2.7, 1H), 4.17-4.11 (m, 1H), 4.02 (dd, J=13.8, 6.8, 1H), 3.76-3.61 (m, 2H), 3.33 (s, 3H), 3.16-3.07 (m, 2H), 3.03 (s, 6H), 2.96-2.90 (m, 2H), 2.89 (s, 3H), 2.86-2.69 (m, 2H), 2.53-2.42 (m, 3H), 2.23-2.18 (m, 1H), 1.78-1.71 (m, 2H).

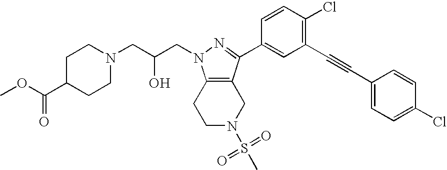

### Example 127

### 1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid methyl ester

HPLC: Rₜ=5.36. MS (ESI): mass calcd. for C₃₁H₃₄Cl₂N₄O₅S, 645.6; m/z found, 646.4 [M+H]⁺. ¹H NMR (CDCl₃): 7.79 (d, J=1.8, 1H), 7.54-7.45 (m, 4H), 7.35 (dt, J=2.1, 8.6, 2H), 4.55 (dd, J=22.8, 14.5, 2H), 4.17 (dd, J=13.8, 2.8, 1H), 4.14-4.08 (m, 1H), 3.98 (dd, J=13.8, 6.8, 1H), 3.74-3.60 (m, 2H), 3.68 (s, 3H), 3.06 (dt, J=5.5, 16.1, 1H), 2.94-2.87 (m, 2H), 2.88 (s, 3H), 2.79-2.76 (br m, 1H), 2.44-2.29 (m, 4H), 2.08-2.03 (br m, 1H), 1.92-1.88 (br m, 3H), 1.80-1.63 (m, 2H).

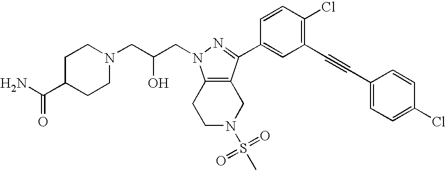

### Example 128

### 1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid amide

HPLC: Rₜ=4.97. MS (ESI): mass calcd. for C₃₀H₃₃Cl₂N₅O₄S, 630.6; m/z found, 631.3 [M+H]⁺. ¹H NMR (CDCl₃): 7.78 (d, J=1.6, 1H), 7.54-7.51 (m, 2H), 7.48-7.47 (m, 2H), 7.37-7.33 (m, 2H), 5.63 (br s, 1H), 5.53 (br s, 1H), 4.54 (dd, J=20.0, 14.5, 2H), 4.17 (dd, J=13.7, 2.8, 1H), 4.14-4.08 (m, 1H), 3.98 (dd, J=13.7, 6.6, 1H), 3.73-3.59 (m, 2H), 3.09-2.84 (m, 4H), 2.88 (s, 3H), 2.44-2.26 (m, 4H), 2.20-2.11 (m, 1H), 2.06-1.99 (m, 1H), 1.89-1.63 (m, 4H).

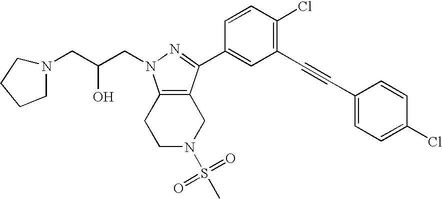

### Example 129

### 1-{3-[4-Chloro-3-(4-chloro-phenylethynyl)-phenyl]-5-methanesulfonyl-4,5,6,7-tetrahydro-pyrazolo[4,3-c]pyridin-1-yl}-3-pyrrolidin-1-yl-propan-2-ol

HPLC: Rₜ=5.53. MS (ESI): mass calcd. for C₂₈H₃₀Cl₂N₄O₃S, 573.6; m/z found, 574.3 [M+H]⁺. ¹H NMR (CDCl₃): 7.79 (d, J=1.9, 1H), 7.54-7.51 (m, 2H), 7.50-7.46 (m, 2H), 7.36-7.33 (m, 2H), 4.54 (dd, J=20.2, 14.5, 2H), 4.18 (dd, J=13.8, 2.8, 1H), 4.13-4.07 (br m, 2H), 3.99 (dd, J=13.8, 7.0, 1H), 3.72-3.58 (m, 2H), 3.08-3.01 (m, 1H), 2.94-2.85 (m, 1H), 2.87 (s, 3H), 2.66-2.61 (m, 3H), 2.53-2.47 (m, 2H), 2.43 (dd, J=12.0, 4.4, 1H), 1.80-1.73 (m, 6H).

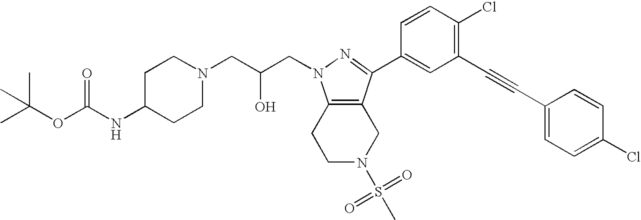

### Example 130

### [1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester

HPLC: Rₜ=5.51. MS (ESI): mass calcd. for C₃₄H₄₁Cl₂N₅O₅S, 702.7; m/z found, 703.5 [M+H]⁺. ¹H NMR (CDCl₃): 7.78 (d, J=1.6, 1H), 7.52 (d, J=8.6, 2H), 7.48-7.46 (m, 2H), 7.35 (d, J=8.6, 2H), 4.54 (dd, J=20.6, 14.5, 2H), 4.48-4.45 (br s, 1H), 4.16 (dd, J=13.8, 2.7, 1H), 4.12-4.07 (br m, 1H), 3.97 (dd, J=13.8, 6.8, 1H), 3.72-3.59 (m, 2H), 3.46 (br s, 1H), 3.07-3.01 (m, 1H), 2.92-2.84 (m, 2H), 2.87 (s, 3H), 2.76-2.70 (br m, 1H), 2.44-2.34 (m, 3H), 2.11 (t, J=11.1, 1H), 1.96-1.88 (br m, 2H), 1.44 (s, 9H), 1.41-1.31 (m, 3H).

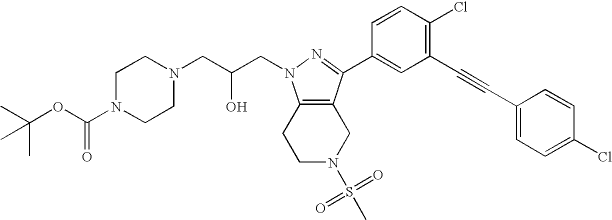

### Example 131

### 4-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperazine-1-carboxylic acid tert-butyl ester

HPLC: Rₜ=5.57. MS (ESI): mass calcd. for C₃₃H₃₉Cl₂N₅O₅S, 688.7; m/z found, 689.4 [M+H]⁺. ¹H NMR (CDCl₃): 7.78 (d, J=1.5, 1H), 7.55-7.51 (m, 2H), 7.49-7.47 (m, 2H), 7.37-7.34 (m, 2H), 4.55 (dd, J=19.9, 14.5, 2H), 4.22-4.09 (m, 3H), 4.00 (dd, J=13.6, 6.5, 1H), 3.74-3.61 (m, 2H), 3.47-3.38 (br m, 4H), 3.08-3.01 (m, 1H), 2.95-2.87 (m, 1H), 2.89 (s, 3H), 2.61-2.54 (m, 2H), 2.49-2.35 (m, 4H), 1.45 (s, 9H).

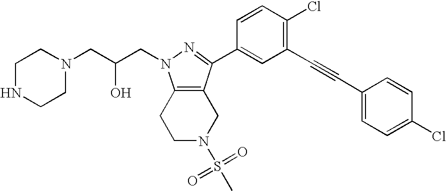

### Example 132

### 1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperazin-1-yl-propan-2-ol

To a solution of 4-(3-{3-[4-chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperazine-1-carboxylic acid tert-butyl ester (117 mg, 0.161 mmol) in CH₂Cl₂ (1.1 mL) was added TFA (0.5 mL). The reaction mixture was stirred at rt for 1 h and then concentrated to give a brown oil. Purification (SiO₂; 0-10% 2 M NH₃ in MeOH/CH₂Cl₂) afforded the title compound as a clear oil (80 mg, 84%). MS (ESI): mass calcd. for C₂₈H₃₁Cl₂N₅O₃S, 588.6; m/z found, 589.3 [M+H]⁺.

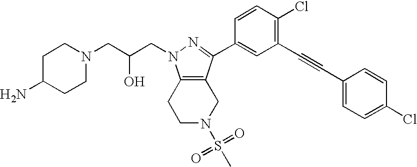

### Example 133

### 1-(4-Amino-piperidin-1-yl)-3-{3-[4-chloro-3-(4-chloro-phenylethynyl)-phenyl]-5-methanesulfonyl-4,5,6,7-tetrahydro-pyrazolo[4,3-c]pyridin-1-yl}-propan-2-ol

This compound was prepared from [1-(3-{3-[4-chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester according to the method described in Example 132. MS (ESI): mass calcd. for C₂₉H₃₃Cl₂N₅O₃S, 602.6; m/z found, 603.4 [M+H]⁺.

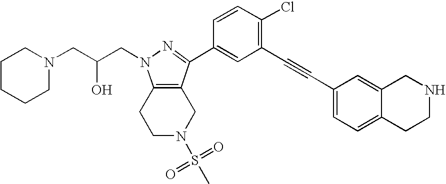

### Example 134

### 1-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-3-piperidin-1-yl-propan-2-ol

A. 7-Bromo-3,4-dihydro-1H-isoquinoline-2-carboxylic acid tert-butyl ester. To a solution of 7-bromo-1,2,3,4-tetrahydroisoquinoline hydrochloride (4.0 g, 16.1 mmol) and Et₃N (6.7 mL, 48.3 mmol) in CH₂Cl₂ (500 mL) was added di-tert-butyl-dicarbonate (4.2 g, 19.2 mmol). After 18 h at rt, the mixture was concentrated and the product was purified on SiO₂ (EtOAc/hexanes) to afford a clear oil (5.00 g, 99%).

B. 7-Trimethylsilanylethynyl-3,4-dihydro-1H-isoquinoline-2-carboxylic acid tert-butyl ester. To a solution of the above bromide (3.0 g, 9.6 mmol), PdCl₂(PPh)₃ (675 mg, 0.96 mmol), and CuI (183 mg, 0.96 mmol) in degassed DMF (50 mL) was added ethynyltrimethylsilane (2.7 mL, 19.2 mmol), followed by Et₃N (4.0 mL, 28.9 mmol). The reaction mixture was stirred under N₂ at 60° C. for 1 h. The mixture was diluted with satd. aq. NaHCO₃ and extracted with EtOAc (×2). The combined organic extracts were washed with water (×3), dried (Na₂SO₄), filtered, and concentrated to give a brown oil. Purification on SiO₂ (EtOAc/hexanes) afforded the desired product (2.78 g, 88%).

C. 7-Ethynyl-3,4-dihydro-1H-isoquinoline-2-carboxylic acid tert-butyl ester. To a solution of the above alkyne (2.7 g, 8.45 mmol) in THF (150 mL) was added tetrabutylammonium fluoride (1.0 M in THF; 16.9 mL, 16.9 mmol). After 1 h at rt, the mixture was diluted with water and extracted with EtOAc (×3). The combined organic extracts were washed with water (×3), dried (Na₂SO₄), filtered, and concentrated to give a brown oil. Purification on SiO₂ (EtOAc/hexanes) provided the title compound as a yellow solid (1.82 g, 84%).

D. 7-[2-Chloro-5-[1-(2-hydroxy-3-piperidin-1-yl-propyl)-5-methanesulfonyl-4,5,6,7-tetrahydro-1H-pyrazolo[4,3-c]pyridin-3-yl]-phenylethynyl]-3,4-dihydro-1H-isoquinoline-2-carboxylic acid tert-butyl ester. To a solution of 1-[7-(4-chloro-3-iodo-phenyl)-2-methanesulfonyl-2,3,4,5-tetrahydro-1H-[2]pyridin-5-yl]-3-piperidin-1-yl-propan-2-ol (25 mg, 0.04 mmol), PdCl₂(PPh)₃ (3 mg, 0.004 mmol), and CuI (1 mg, 0.004 mmol) in degassed THF (1 mL) was added the above alkyne (17 mg, 0.065 mmol) in degassed THF (200 μL), followed by Et₃N (200 μL, 0.129 mmol). The reaction mixture was stirred under N₂ at 50° C. for 18 h. Additional alkyne (17 mg, 0.065 mmol) in degassed THF (200 μL) was added and the mixture was heated at 50° C. for an additional 1 h. The mixture was diluted with satd. aq. NaHCO₃ and extracted with CH₂Cl₂ (×3). The combined organic extracts were dried (Na₂SO₄) and concentrated to give a brown oil. Purification by preparative thin layer chromatography (SiO₂, 3% MeOH/NH₃/CH₂Cl₂) afforded the desired product as a yellow oil (26 mg, 87%).

E. 1-[3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-3-piperidin-1-yl-propan-2-ol. To a solution of 7-{2-chloro-5-[1-(2-hydroxy-3-piperidin-1-yl-propyl)-5-methanesulfonyl-4,5,6,7-tetrahydro-1H-pyrazolo[4,3-c]pyridin-3-yl]-phenylethynyl}-3,4-dihydro-1H-isoquinoline-2-carboxylic acid tert-butyl ester (23 mg, 0.033 mmol) in CH₂Cl₂ (1 mL), was added HCl (1.0 M in Et₂O; 100 μL, 0.100 mmol). After 1 h at rt, additional CH₂Cl₂ (1 mL) and HCl (1.0 M in Et₂O; 1 mL, 1.00 mmol) were added and the mixture was stirred for an additional 1 h. HCl (4.0 M in dioxane; 400 μL, 1.6 mmol) was added and the mixture was stirred for an additional 1 h, then was concentrated to afford the desired product. HPLC: Rₜ=4.31. MS (ESI): mass calculated for C₃₂H₃₈ClN₅O₃S, 607.2; m/z found, 608.3 [M+H]⁺. ¹H NMR (CD₃OD): 7.87 (s, 1H), 7.60 (dd, J=17.9, 8.4, 2H), 7.52-7.46 (m, 2H), 7.32 (d, J=7.9, 1H), 4.53 (s, 2H), 4.50 (br s, 1H), 4.40 (s, 2H), 4.21 (dd, J=12.6, 4.8, 2H), 3.68-3.64 (m, 4H), 3.66 (s, 3H), 3.62-3.50 (m, 4H), 3.18 (dd, J=14.1, 8.2, 3H), 3.11-2.97 (m, 6H), 1.98-1.71 (m, 4H).

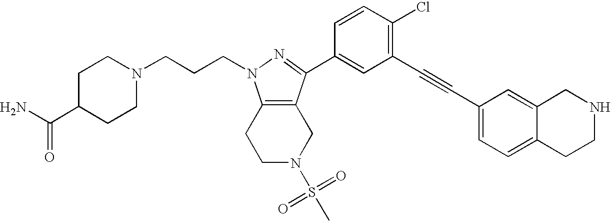

### Example 135

### 1-(3-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-propyl)-piperidine-4-carboxylic acid amide

A. 1-{3-[3-(4-Chloro-3-iodo-phenyl)-5-methanesulfonyl-4,5,6,7-tetrahydro-pyrazolo[4,3-c]pyridin-1-yl]-propyl}-piperidine-4-carboxylic acid amide. The title compound was prepared using methods analogous to those described in Example 1, Steps A-D, substituting isonipecotamide for morpholine in Step D.

B. 7-(5-[1-[3-(4-Carbamoyl-piperidin-1-yl)-propyl]-5-methanesulfonyl-4,5,6,7-tetrahydro-1H-pyrazolo[4,3-c]pyridin-3-yl]-2-chloro-phenylethynyl)-3,4-dihydro-1H-isoquinoline-2-carboxylic acid tert-butyl ester. The title compound was prepared using methods analogous to those described in Example 134, Steps A-D.

C. 1-(3-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl-pr piperidine-4-carboxylic acid amide. To a 0° C. solution of 7-(5-{1-[3-(4-carbamoyl-piperidin-1-yl)-propyl]-5-methanesulfonyl-4,5,6,7-tetrahydro-1H-pyrazolo[4,3-c]pyridin-3-yl}-2-chloro-phenylethynyl)-3,4-dihydro-1H-isoquinoline-2-carboxylic acid tert-butyl ester (80 mg, 0.109 mmol) in CH₂Cl₂ (3 mL) was added TFA (100 μL, 1.32 mmol). After 30 min at 0° C., additional TFA (1 mL, 13.2 mmol) was added. After 30 min, the mixture was diluted with satd. aq. NaHCO₃ and extracted with CH₂Cl₂. The organic layer was removed and aqueous layer was decanted to leave an oil which was combined with the organic layer and concentrated. The residue was dissolved in MeOH, sonicated, and decanted from precipitates. The MeOH solution was concentrated to afford the desired product (10 mg, 14%). HPLC: Rₜ=4.14. MS (ESI): mass calcd. for C₃₃H₃₉ClN₆O₃S, 634.3; m/z found, 635.3 [M+H]⁺. ¹H NMR (CD₃OD): 7.86 (d, J=1.8, 1H), 7.59 (dt, J=14.2, 5.2, 2H), 7.53-7.48 (m, 1H), 7.46 (s, 1H), 7.32 (d, J=8.0, 1H), 4.51 (s, 2H), 4.40 (s, 2H), 4.23 (t, J=6.4, 2H), 3.69-3.60 (m, 4H), 3.53 (t, J=6.4, 2H), 3.24-3.13 (m, 4H), 3.04-2.90 (m, 4H), 2.98 (s, 3H), 2.59-2.47 (m, 1H), 2.39-2.28 (m, 2H), 2.07 (d, J=13.9, 2H), 1.96-1.82 (m, 2H).

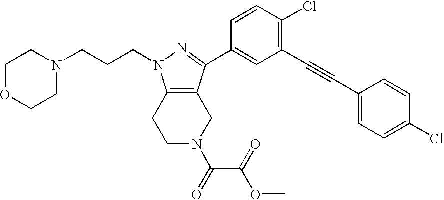

### Example 136

### [3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-oxo-acetic acid methyl ester

A. 3-(4-Chloro-3-iodo-phenyl)-1,4,6,7-tetrahydro-pyrazolo[4,3-c]pyridine-5-carboxylic acid tert-butyl ester. This compound was prepared according to the method described in Intermediate 1, Step B, substituting 4-oxo-piperidine-1-carboxylic acid tert-butyl ester for 1-methanesulfonyl-piperidin-4-one. HPLC: Rₜ=7.69. MS (ESI): mass calcd. for C₁₇H₁₉ClIN₃O₂, 459.7; m/z found, 460.2 [M+H]⁺. ¹H NMR (CDCl₃): 8.08 (br s, 1H), 7.45 (br m, 2H), 4.63 (br s, 2H), 3.73 (br m, 2H), 2.75 (br m, 2H), 1.49 (s, 9H).

B. 3-(4-Chloro-3-iodo-phenyl)-1-(2-cyano-ethyl)-1,4,6,7-tetrahydro-pyrazolo[4,3-c]pyridine-5-carboxylic acid tert-butyl ester. To a solution of the above pyrazole (2.8 g, 6.09 mmol) and acrylonitrile (14 mL) in THF (14 mL) was added 1% aq. NaOH (5.6 mL) dropwise. The reaction mixture was stirred at rt for 8 h, diluted with satd. aq. NH₄Cl, and extracted with CH₂Cl₂ (3×). The combined organic extracts were dried (Na₂SO₄), filtered, and concentrated to give a brown paste. Recrystallization from EtOH afforded the title compound as a white solid (2.24 g, 72%). HPLC: Rₜ=7.91. MS (ESI): mass calcd. for C₂₀H₂₂CIIN₄O₂, 512.8; m/z found, 514.2 [M+H]⁺. ¹H NMR (CDCl₃): 8.18 (s, 1H), 7.51-7.46 (br m, 2H), 4.60 (br s, 2H), 4.30 (t, J=6.5, 2H), 3.76-3.72 (br m, 2H), 2.98 (t, J=6.5, 2H), 2.80-2.78 (br m, 2H), 1.49 (s, 9H).

C. 3-(4-Chloro-3-iodo-phenyl)-1-(3-morpholin-4-yl-propyl)-1,4,6,7-tetrahydro-pyrazolo[4,3-c]pyridine-5-carboxylic acid tert-butyl ester. To a −78 LC solution of the above nitrile (2.39 g, 4.66 mmol) in CH₂Cl₂ (23 mL) was added DIBAL-H (1.5 M in toluene; 7.75 mL, 5.17 mmol) dropwise. The mixture was stirred at −78 LC for 30 min and then warmed to rt for 30 min. The reaction mixture was cooled to −78 LC and MeOH (15 mL) was added dropwise. The mixture was warmed to rt over 1 h and 1 N H₂SO₄ (10 mL) was added. After 1 h, the cloudy mixture was diluted with satd. aq. potassium sodium tartrate and extracted with CH₂Cl₂ (3×). The combined organic extracts were washed with brine, dried (Na₂SO₄), filtered and concentrated to give a white foam, which was used directly in the next reaction. The reductive amination was conducted in a similar manner to that reported in Intermediate 1, Step D, to provide the title compound as a white solid (1.43 g, 52%). HPLC: Rₜ=5.42. MS (ESI): mass calcd. for C₂₄H₃₂CIIN₄O₃, 586.9; m/z found, 588.3 [M+H]⁺. ¹H NMR (CDCl₃): 8.18 (d, J=2.1, 1H), 7.54-7.49 (m, 2H), 4.60 (br s, 2H), 4.08 (t, J=6.8, 2H), 3.76-3.71 (br m, 2H), 3.70 (br t, J=4.5, 4H), 2.74 (br t, J=5.4, 2H), 2.43-2.38 (br m, 4H), 2.32 (t, J=6.8, 2H), 2.09-2.01 (m, 2H), 1.49 (s, 9H).

D. 3-[4-Chloro-3-(4-chloro-phenylethynyl)-phenyl]-1-(3-morpholin-4-yl-propyl)-1,4,6,7-tetrahydro-pyrazolo[4,3-c]pyridine-5-carboxylic acid tert-butyl ester. This compound was prepared from the above iodide according to the method described in Intermediate 1, Step E, substituting 4-chloro-ethynylbenzene for TMSA. HPLC: Rₜ=6.43. MS (ESI): mass calcd. for C₃₂H₃₆Cl₂N₄O₃, 595.6; m/z found, 596.4 [M+H]⁺. ¹H NMR (CDCl₃): 7.85 (s, 1H), 7.65-7.42 (m, 2H), 7.51 (d, J=8.3, 2H), 7.37-7.32 (m, 2H), 4.65 (br s, 2H), 4.09 (t, J=6.8, 2H), 3.78-3.71 (br m, 2H), 3.69 (br t, J=4.5, 4H), 2.75 (br t, J=5.2, 2H), 2.43-2.37 (br m, 4H), 2.32 (t, J=6.8, 2H), 2.10-2.02 (m, 2H), 1.49 (s, 9H).

E. 3-[4-Chloro-3-(4-chloro-phenylethynyl)-phenyl]-1-(3-morpholin-4-yl-propyl)-4,5,6,7-tetrahydro-1H-pyrazolo[4,3-c]pyridine. To a solution of the above carbamate (1.45 g, 2.43 mmol) in CH₂Cl₂ (20 mL) was added TFA (5 mL) dropwise. The reaction mixture was stirred at rt for 3 h and then concentrated to give an orange oil. Purification (SiO₂; 0-10% 2 M NH₃ in MeOH/CH₂Cl₂) provided the title compound as a clear oil (1.11 g, 92%). HPLC: Rₜ=4.53. MS (ESI): mass calcd. for C₂₇H₂₈Cl₂N₄O, 495.5; m/z found, 496.3 [M+H]⁺. ¹H NMR (CDCl₃): 7.82 (d, J=2.1, 1H), 7.52-7.49 (m, 3H), 7.43 (d, J=8.4, 1H), 7.34 (dd, J=6.6, 2.0, 2H), 4.10-4.06 (m, 4H), 3.70 (t, J=4.6, 4H), 3.18 (t, J=5.4, 2H), 2.92 (br s, 1H), 2.72 (t, J=5.7, 2H), 2.42 (br m, 4H), 2.34 (t, J=6.9, 2H), 2.09-2.03 (m, 2H).

F. [3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-oxo-acetic acid methyl ester. To a solution of the above amine (100 mg, 0.202 mmol) and pyridine (36 μL, 0.444 mmol) in CH₂Cl₂ (2.0 mL) was added methyl chlorooxoacetate (20 μL, 0.222 mmol) dropwise. The reaction mixture was stirred at rt for 1.5 h, diluted with satd. aq. NaHCO₃, and extracted with CH₂Cl₂ (3×). The combined organic extracts were dried (Na₂SO₄), filtered, and concentrated to give a yellow oil. Purification (SiO₂; 0-3% 2 M NH₃ in MeOH/CH₂Cl₂) provided the title compound as a clear oil (104 mg, 89%). HPLC: Rₜ=5.36. MS (ESI): mass calcd. for C₃₀H₃₀Cl₂N₄O₄, 581.5; m/z found, 582.4 [M+H]⁺. ¹H NMR (CDCl₃, 2:1 mixture of rotamers): 7.83 (d, J=2.1, 0.7H), 7.78 (d, J=2.0, 0.3H), 7.57-7.40 (m, 4H), 7.35 (dd, J=8.6, 2.0, 2H), 4.83 (s, 1.3H), 4.66 (s, 0.7H), 4.15-4.08 (m, 2H), 3.98 (t, J=5.9, 1H), 3.93 (s, 2H), 3.87 (s, 1H), 3.75-3.69 (m, 5H), 2.91 (t, J=5.6, 1H), 2.86 (t, J=5.8, 1H), 2.40 (br m, 4H), 2.35-2.29 (m, 2H), 2.11-2.06 (m, 2H).

### Alternative Synthesis of 3-(4-Chloro-3-iodo-phenyl)-1,4,6,7-tetrahydro-pyrazolo[4,3-c]pyridine-5-carboxylic acid tert-butyl ester

A. 4-Morpholin-4-yl-3,6-dihydro-2H-pyridine-1-carboxylic acid tert-but I ester. In a 3-L round-bottom flask equipped with a Dean-Stark trap, a reflux condenser and an internal thermocouple, N-Boc-piperidone (200 g, 1.0 mol, 1.0 equiv), toluene (2 L), morpholine (92 mL, 1.05 mol, 1.05 equiv), and p-toluenesulfonic acid (1.0 g, 0.005 mmol, 0.5% equiv) were added sequentially. The reaction solution was refluxed under N₂ for 16 h (about 18 mL water was collected). The solvent was evaporated and the residue was used directly in the next reaction (colorless oil, ˜270 g, 100%).

B. 4-Chloro-3-iodo-benzoyl chloride. In a 5-L round-bottom flask equipped with a magnetic stirring bar and a gas scrubber, 4-chloro-3-iodobenzoic acid (275 g, 0.975 mol, 1.0 equiv) was suspended in CH₂Cl₂ (3 L) and DMF (2 mL, 0.026 mol, 2.5% equiv) was added. Under N₂ at 0° C., oxalyl chloride (93.5 mL, 1.1 mol, 1.1 equiv) was added dropwise over 1 h. The ice bath was removed and the reaction solution was stirred at rt for 16 h. The solvent was evaporated and the residue was used directly in the next reaction (−290 g, 100%).

C. 3-(4-Chloro-3-iodo-phenyl)-1,4,6,7-tetrahydro-pyrazolo[4,3-c]pyridine-5-carboxylic acid tert-butyl ester. 4-Morpholin-4-yl-3,6-dihydro-2H-pyridine-1-carboxylic acid tert-butyl ester (270 g, 1.0 mol, 1.0 equiv) was dissolved in CH₂Cl₂ (1.6 L) and then Et₃N (209 mL, 1.5 mol, 1.5 equiv) was added. At 0° C. under N₂, a solution of 4-chloro-3-iodo-benzoyl chloride (290 g, 1.0 mol, 1.0 equiv) in CH₂Cl₂ (400 mL) was added over 30 min. The ice bath was then removed and the reaction solution was stirred at rt for 3 h. All the volatile solvents were removed in vacuo and the residue was re-dissolved in EtOH (1.5 L). At 0° C., anhydrous NH₂NH₂ (47 mL, 1.5 mol, 1.5 equiv) was added over 30 min (exothermic reaction). The reaction solution was stirred at rt for 16 h. The precipitated white solid was collected by filtration and washed with cold EtOH to afford the desired pyrazole product (white solid, ˜333 g, 0.73 mol, >95% purity, 73%). The mother liquor was concentrated and was partitioned between CH₂Cl₂ and H₂O. Emulsion was observed due to the low solubility of the desired product in CH₂Cl₂. The insoluble solid was collected by filtration to provide an additional portion of the desired product. The organic layer was warmed slightly to assist phase separation. The organic layer was washed with water (3×), dried, and concentrated. The crude product (filtered material plus residue) was recrystallized from hot CH₃CN to give the title compound (74 g, 0.16 mmol, 16%). The total yield was 89% for the three steps.

The compounds in Examples 137-142 were prepared using methods analogous to those described in Example 136, substituting the appropriate acid chloride for methyl chlorooxoacetate in Step F.

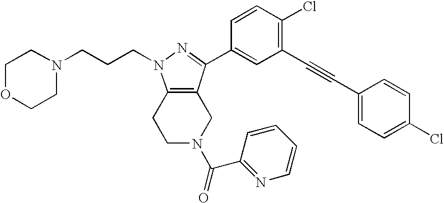

### Example 137

### [3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-pyridin-2-yl-methanone

HPLC: Rₜ=5.48. MS (ESI): mass calcd. for C₃₃H₃₁Cl₂N₅O₂, 600.6; m/z found, 601.5 [M+H]⁺. ¹H NMR (CDCl₃): 8.60 (t, J=4.8, 1H), 7.90 (d, J=2.0, 1H), 7.87-7.77 (m, 1H), 7.76-7.70 (m, 1H), 7.63 (dd, J=8.4, 2.1, 1H), 7.56-7.46 (m, 3H), 7.42-7.33 (m, 4H), 4.99 (s, 1H), 4.14-4.08 (m, 3H), 3.86 (t, J=5.6, 1H), 3.72-3.68 (m, 4H), 2.98-2.90 (m, 2H), 2.44-2.38 (br m, 4H), 2.37-2.31 (m, 2H), 2.13-2.05 (m, 2H).

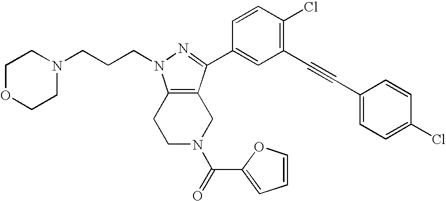

### Example 138

### [3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-furan-2-yl-methanone

HPLC: Rₜ=5.27. MS (ESI): mass calcd. for C₃₂H₃₀Cl₂N₄O₃, 589.5; m/z found, 590.4 [M+H]⁺. ¹H NMR (CDCl₃): 7.86 (s, 1H), 7.54-7.50 (m, 3H), 7.46 (d, J=8.4, 2H), 7.37-7.33 (m, 2H), 7.08 (d, J=3.2, 1H), 6.52-6.50 (m, 1H), 4.95 (br s, 2H), 4.11 (t, J=6.9, 2H), 4.05 (t, J=5.6, 2H), 3.70 (t, J=4.6, 4H), 2.96-2.90 (br m, 2H), 2.45-2.39 (br m, 4H), 2.34 (t, J=6.8, 2H), 2.12-2.06 (m, 2H).

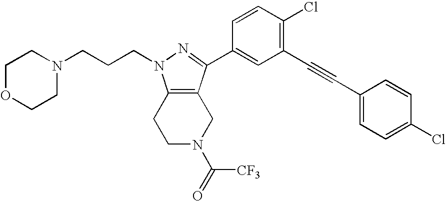

### Example 139

### 1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2,2,2-trifluoro-ethanone

MS (ESI): mass calcd. for C₂₉H₂₇Cl₂F₃N₄O₂, 591.5; m/z found, 592.4 [M+H]⁺.

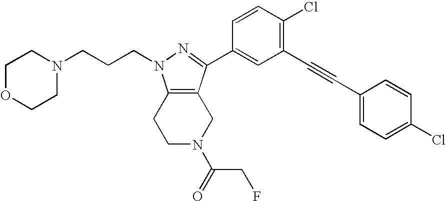

### Example 140

### 1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-fluoro-ethanone

MS (ESI): mass calcd. for C₂₉H₂₉Cl₂FN₄O₂, 555.5; m/z found, 556.4 [M+H]⁺.

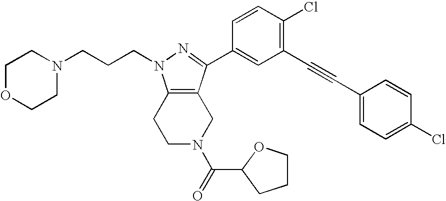

### Example 141

### [3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-(tetrahydro-furan-2-yl)-methanone

MS (ESI): mass calcd. for C₃₂H₃₄Cl₂N₄O₃, 593.6; m/z found, 594.4 [M+H]⁺.

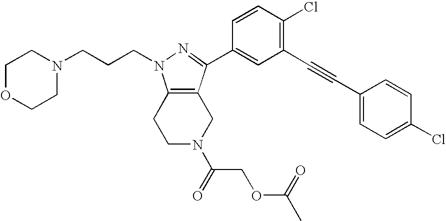

### Example 142

### Acetic acid 2-[3-[4-chloro-3-(4-chloro-phenylethynyl)-phenyl]-1-(3-morpholin-4-yl-propyl)-1,4,6,7-tetrahydro-pyrazolo[4,3-c]pyridin-5-yl]-2-oxo-ethyl ester

MS (ESI): mass calcd. for C₃₁H₃₂Cl₂N₄O₄, 595.5; m/z found, 596.4 [M+H]⁺.

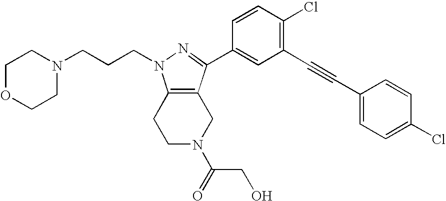

### Example 143

### 1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-hydroxy-ethanone

To a slurry of acetic acid 2-[3-[4-chloro-3-(4-chloro-phenylethynyl)-phenyl]-1-(3-morpholin-4-yl-propyl)-1,4,6,7-tetrahydro-pyrazolo[4,3-c]pyridin-5-yl]-2-oxo-ethyl ester (105 mg, 0.176 mmol) in 2:1 MeOH/H₂O (1.5 mL) was added NaOH (14 mg, 0.352 mmol). The reaction mixture was stirred at rt for 2 h, diluted with satd. aq. NaHCO₃, and extracted with CH₂Cl₂ (3×). The combined organic extracts were dried (Na₂SO₄), filtered, and concentrated to give a yellow oil. Purification (SiO₂; 0-5% 2 M NH₃ in MeOH/CH₂Cl₂) provided the title compound as a white solid (62 mg, 64%). MS (ESI): mass calcd. for C₂₉H₃₀Cl₂N₄O₃, 553.5; m/z found, 554.4 [M+H]⁺.

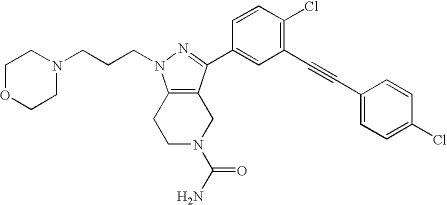

### Example 144

### 3-[4-Chloro-3-(4-chloro-phenylethynyl)-phenyl]-1-(3-morpholin-4-yl-propyl)-1,4,6,7-tetrahydro-pyrazolo[4,3-c]pyridine-5-carboxylic acid amide

This compound was prepared according to the methods described in Example 136, substituting trimethylsilyl isocyanate for methyl chlorooxoacetate in Step F. HPLC: Rₜ=4.81. MS (ESI): mass calcd. for C₂₈H₂₉Cl₂N₅O₂, 538.5; m/z found, 539.3 [M+H]⁺. ¹H NMR (CDCl₃): 7.79 (d, J=2.0, 1H), 7.52-7.50 (m, 2H), 7.43 (d, J=8.4, 2H), 7.34 (d, J=8.5, 2H), 4.90 (s, 2H), 4.58 (s, 2H), 4.12-4.06 (m, 2H), 3.74 (t, J=5.6, 2H), 3.68 (t, J=4.5, 4H), 2.77 (t, J=5.4, 2H), 2.39 (br m, 4H), 2.32 (t, J=6.8, 2H), 2.08-1.99 (m, 2H).

The compounds in Examples 145-149 were prepared using methods analogous to those described in Example 2. The alkynes used as starting materials for Examples 145-149 are described in the preceding examples.

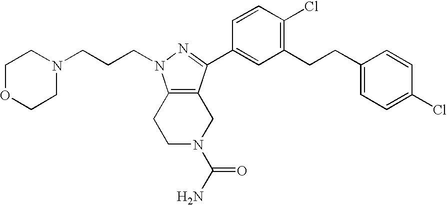

### Example 145

### 3-{4-Chloro-3-[2-(4-chloro-phenyl)-ethyl]-phenyl}-1-(3-morpholin-4-yl-propyl)-1,4,6,7-tetrahydro-pyrazolo[4,3-c]pyridine-5-carboxylic acid amide

HPLC: Rₜ=5.04. MS (ESI): mass calcd. for C₂₈H₃₃Cl₂N₅O₂, 542.5; m/z found, 543.5 [M+H]⁺. ¹H NMR (CDCl₃): 7.43 (d, J=1.8, 1H), 7.36 (dd, J=8.3, 2.0, 2H), 7.26-7.23 (m, 2H), 7.16-7.13 (m, 2H), 4.61 (br s, 2H), 4.47 (s, 2H), 4.09 (t, J=6.9, 2H), 3.76 (t, J=5.7, 2H), 3.69 (t, J=4.6, 4H), 3.06-3.01 (m, 2H), 2.95-2.90 (m, 2H), 2.78 (t, J=5.7, 2H), 2.42-2.38 (br m, 4H), 2.33 (t, J=6.8, 2H), 2.13-2.05 (m, 2H).

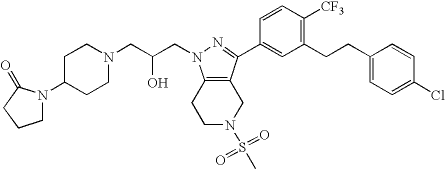

### Example 146

### 1-{1-[3-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-pyrrolidin-2-one

HPLC: Rₜ=5.33. MS (ESI): mass calcd. for C₃₄H₄₁ClF₃N₅O₄S, 708.3; m/z found, 709.5 [M+H]⁺. ¹H NMR (CDCl₃): 7.68 (d, J=8.2, 1H), 7.54 (s, 1H), 7.46 (d, J=8.2, 1H), 7.28-7.25 (m, 2H), 7.17-7.13 (m, 2H), 4.49 (dd, J=18.5, 14.6, 2H), 4.19 (dd, J=13.8, 2.8, 1H), 4.16-4.09 (m, 1H), 4.04-3.96 (m, 2H), 3.73-3.62 (m, 2H), 3.34 (t, J=7.0, 2H), 3.13-2.99 (m, 4H), 2.95-2.86 (m, 5H), 2.88 (s, 3H), 2.50-2.42 (m, 3H), 2.39 (t, J=8.2, 2H), 2.05-1.97 (m, 3H), 1.76-1.62 (m, 4H).

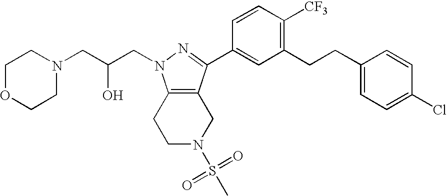

### Example 147

### 1-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-3-morpholin-4-yl-propan-2-ol

HPLC: Rₜ=5.56. MS (ESI): mass calcd. for C₂₉H₃₄ClF₃N₄O₄S, 627.1; m/z found, 628.4 [M+H]⁺. ¹H NMR (CDCl₃): 7.68 (d, J=8.2, 1H), 7.53 (s, 1H), 7.46 (d, J=8.2, 1H), 7.28-7.25 (m, 2H), 7.17-7.14 (m, 2H), 4.49 (dd, J=19.4, 14.5, 2H), 4.21 (dd, J=13.7, 2.8, 1H), 4.18-4.13 (m, 1H), 4.02 (dd, J=13.6, 6.7, 1H), 3.75-3.62 (m, 6H), 3.12-3.02 (m, 3H), 2.96-2.87 (m, 4H), 2.88 (s, 3H), 2.66-2.60 (m, 2H), 2.50-2.40 (m, 4H).

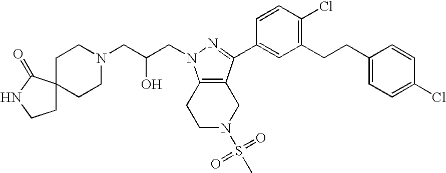

### Example 148

### 8-[3-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-2-hydroxy-propyl]-2,8-diaza-spiro[4.5]decan-1-one

HPLC: Rₜ=5.21. MS (ESI): mass calcd. for C₃₂H₃₉Cl₂N₅O₄S, 660.7; m/z found, 661.5 [M+H]⁺. ¹H NMR (CDCl₃): 7.40-7.30 (m, 4H), 7.23 (d, J=7.5, 2H), 7.13 (d, J=7.9, 2H), 4.43 (dd, J=17.8, 15.0, 2H), 4.17-4.10 (m, 2H), 4.01-3.94 (m, 1H), 3.70-3.60 (m, 2H), 3.35-3.33 (m, 1H), 3.30 (t, J=6.8, 2H), 3.07-2.99 (m, 3H), 2.93-2.78 (m, 5H), 2.87 (s, 3H), 2.45-2.38 (m, 2H), 2.30 (br t, J=10.8, 2H), 2.10 (br t, J=11.7, 2H), 2.02 (t, J=6.9, 2H), 1.95-1.82 (m, 2H).

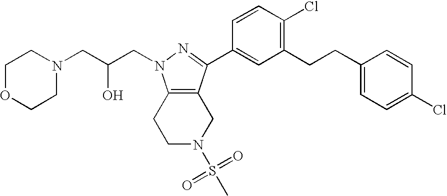

### Example 149

### 1-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-3-morpholin-4-yl-propan-2-ol

HPLC: Rₜ=5.47. MS (ESI): mass calcd. for C₂₈H₃₀Cl₂N₄O₄S, 589.6; m/z found, 590.2 [M+H]⁺. ¹H NMR (CDCl₃): 7.78 (d, J=1.5, 1H), 7.54-7.50 (m, 2H), 7.48-7.46 (m, 2H), 7.36-7.33 (m, 2H), 4.53 (dd, J=18.3, 14.5, 2H), 4.18 (dd, J=13.3, 2.9, 1H), 4.17-4.10 (m, 1H), 3.99 (dd, J=13.7, 6.6, 1H), 3.71-3.60 (m, 6H), 3.07-2.99 (m, 1H), 2.93-2.85 (m, 2H), 2.87 (s, 3H), 2.65-2.58 (m, 2H), 2.48-2.38 (m, 4H).

Unless otherwise specified, the compounds in Examples 150-621 were prepared as free base, hydrochloride salt, trifluoroacetic acid salt, citric acid, or formic acid salt forms.

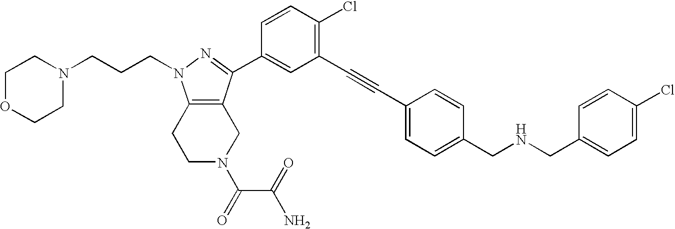

### Example 150

### 2-[3-(4-Chloro-3-{[4-({[(4-chlorophenyl)methyl]amino}methyl)phenyl]ethynyl}phenyl)-1-(3-morpholin-4-ylpropyl)-1,4,6,7-tetrahydro-5H-pyrazolo[4,3-c]pyridin-5-yl]-2-oxoacetamide

A. 3-(4-Chloro-3-iodo-phenyl)-1-(3-hydroxy-propyl)-1,4,6,7-tetrahydro-pyrazolo[4,3-c]pyridine-5-carboxylic acid tert-butyl ester. A mixture of 3-(4-chloro-3-iodo-phenyl)-1,4,6,7-tetrahydro-pyrazolo[4,3-c]pyridine-5-carboxylic acid tert-butyl ester (21.0 mmol) and Cs₂CO₃ (43.8 mmol) in DMF (73 mL) at 0° C., was treated with 3-bromo-propan-1-ol (32.9 mmol). The mixture was stirred at rt for 15 h. The mixture was poured into water and extracted with EtOAc (3×). The combined organic layers were washed satd. aq. NaCl (4×). The organic phase was dried and concentrated, and the resulting oil was purified (SiO₂; 10-50% EtOAc/hexanes) to afford the title compound (74%). MS (ESI): mass calcd. for C₂₀H₂₅CIIN₃O₃, 517.06; m/z found, 518.1 [M+H]⁺.

B. 3-(4-Chloro-3-iodo-phenyl)-1-(3-oxo-propyl)-1,4,6,7-tetrahydro-pyrazolo[4,3-c]pyridine-5-carboxylic acid tert-butyl ester. A solution of the alcohol above (9.8 mmol) and Dess-Martin periodinane (14.7 mmol) in CH₂Cl₂ (43.4 mL) was stirred for 2 h at rt. The mixture was poured into satd. aq. NaHCO₃ and extracted with CH₂Cl₂ (3×). The combined organic layers were dried and concentrated. The resulting oil was passed through a plug of SiO₂ eluting with CH₂Cl₂ and EtOAc. The filtrate was concentrated and used immediately. MS (ESI): mass calcd. for C₂₀H₂₃CIIN₃O₃, 515.05; m/z found, 516.1 [M+H]⁺.

C. 3-(4-Chloro-3-iodo-phenyl)-1-(3-morpholin-4-yl-propyl)-1,4,6,7-tetrahydro-pyrazolo[4,3-c]pyridine-5-carboxylic acid tert-butyl ester. This compound was prepared from the aldehyde above according to the method described for Intermediate 1, Part D. MS (ESI): mass calcd. for C₂₄H₃₂CIIN₄O₃, 586.12; m/z found, 587.2 [M+H]⁺

D. 3-[4-Chloro-3-(4-{[(4-chloro-benzyl)-(2,2,2-trifluoro-acetyl)-amino]-methyl]-phenylethynyl)-phenyl]-1-(3-morpholin-4-yl-propyl)-1,4,6,7-tetrahydro-pyrazolo[4,3-c]pyridine-5-carboxylic acid tert-butyl ester. This compound was prepared according to the method described for Intermediate 1, Part E, substituting N-(4-chloro-benzyl)-N-(4-ethynyl-benzyl)-2,2,2-trifluoro-acetamide for (trimethylsilyl)acetylene. MS (ESI): mass calcd. for C₄₂H₄₄Cl₂F₃N₅O₄, 809.27; m/z found, 810.2 [M+H]⁺.

E. N-(4-Chloro-benzyl)-N-(4-{2-chloro-5-[1-(3-morpholin-4-yl-prolyl)-4,5,6,7-tetrahydro-1H-pyrazolo[4,3-c]pyridin-3-yl phenylethynyl]-benzyl)-2,2,2-trifluoro-acetamide. This compound was prepared according to the method described in Example 136, Part E. MS (ESI): mass calcd. for C₃₇H₃₆Cl₂F₃N₅O₂, 709.22; m/z found, 710.2 [M+H]⁺.

F. N-(4-[5-[5-Aminooxalyl-1-(3-morpholin-4-propyl)-3a,4,5,6,7,7a-hexahydro-1H-pyrazolo[4,3-c]pyridin-3-yl]-2-chloro-phenylethynyl]-benzyl)-N-(4-chloro-benzyl)-2,2,2-trifluoro-acetamide. To N-(4-chloro-benzyl)-N-(4-{2-chloro-5-[1-(3-morpholin-4-yl-propyl)-4,5,6,7-tetrahydro-1H-pyrazolo[4,3-c]pyridin-3-yl]phenylethynyl}-benzyl)-2,2,2-trifluoro-acetamide (0.132 mmol) was added oxalamic acid (0.527 mmol), HATU (0.264 mmol), HOAT (0.5 M in DMF; 530 μL), DMF (0.66 mL), and iPr₂NEt (0.396 mmol). The mixture was stirred at 80° C. for 26.5 h. The mixture was poured into satd. aq. NaHCO₃ (10 mL). The aqueous phase was extracted CH₂Cl₂ (3×10 mL) and EtOAc (2×10 mL). The combined organic layers were dried and concentrated. The resulting oil was purified (SiO₂; 0-5% 0.2 M NH₃ in MeOH/CH₂Cl₂) to afford the title compound as an oil (60%, 0.080 mmol). MS (ESI): mass calcd. for C₃₉H₃₉Cl₂F₃N₆O₄, 782.24; m/z found, 783.2 [M+H]⁺.

G. 2-[3-(4-Chloro-3-{[4-(ff(4-chlorophenyl)methyl]amino]methyl)phenyl]ethynyl}phenyl)-1-(3-morpholin-4-ylpropyl)-1,4,6,7-tetrahydro-5H-pyrazolo[4,3-c]pyridin-5-yl]-2-oxoacetamide. A mixture of N-(4-{5-[5-aminooxalyl-1-(3-morpholin-4-yl-propyl)-4,5,6,7-tetrahydro-1H-pyrazolo[4,3-c]pyridin-3-yl]-2-chloro-phenylethynyl}-benzyl)-N-(4-chloro-benzyl)-2,2,2-trifluoro-acetamide (0.026 mmol) and K₂CO₃ (0.153 mmol) in MeOH/H₂O (2:1, 1.5 mL) was stirred for 30 min. The mixture was poured into H₂O, extracted with CH₂Cl₂ (2×) and EtOAc (2×). The combined organic layers were dried and concentrated. The resulting oil was purified using preparatory HPLC (Method E). MS (ESI): mass calcd. for C₃₇H₃₈Cl₂N₆O₃, 684.24; m/z found, 685.2 [M+H]⁺. ¹H NMR: 7.92-7.81 (m, 1H), 7.68-7.63 (m, 3H), 7.60-7.56 (m, 3H), 7.55-7.52 (m, 2H), 7.50-7.44 (m, 2H), 4.80 (s, 1H), 4.32-4.23 (m, 6H), 4.05-3.91 (m, 4H), 3.78 (t, J=12, 2H), 3.51 (d, J=12, 2H), 3.31-3.23 (m, 6H), 3.15 (dt, J=12, 3, 2H), 3.00-2.87 (m, 2H), 2.42-2.33 (m, 2H).

The compounds in Examples 151-155 were prepared using methods analogous to those described in Example 150, substituting oxalamic acid with the appropriate carboxylic acids (Step F), or substituting oxalamic acid, HATU, HOAt, iPr₂NEt, and DMF with the appropriate sulfonyl chlorides, acid chlorides, or isocyanates in CH₂Cl₂, with exceptions or alterations where noted.

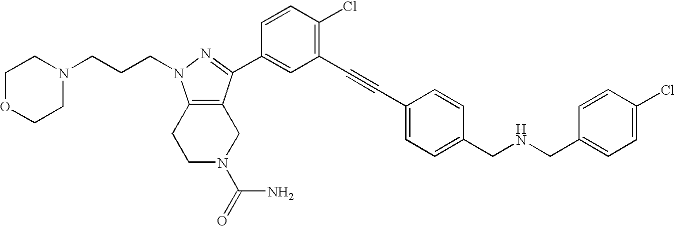

### Example 151

### 3-(4-Chloro-3-{[4-({[(4-chlorophenyl)methyl]amino}methyl)phenyl]ethynyl}phenyl)-1-(3-morpholin-4-ylpropyl)-1,4,6,7-tetrahydro-5H-pyrazolo[4,3-c]pyridine-5-carboxamide

The compound was purified using preparatory HPLC (Method E; HCl salt). MS (ESI): mass calcd. for C₃₆H₃₈Cl₂N₆O₂, 656.24; m/z found, 657.2 [M+H]⁺. ¹H NMR: 7.93 (d, J=1.9, 1H), 7.68-7.64 (m, 3H), 7.63-7.59 (m, 3H), 7.59-7.54 (m, 3H), 7.49-7.45 (m, 2H), 4.76 (s, 1H), 4.35-4.25 (m, 7H), 4.02 (dd, J=13, 2.9, 2H), 3.90 (t, J 5.5, 2H), 3.84 (dd, J=25, 13, 2H), 3.52 (d, J=12, 2H), 3.30-3.25 (m, 4H), 3.17 (dt, J=12, 3.4, 2H), 2.96 (t, J=5.2, 2H), 2.46-2.36 (m, 2H).

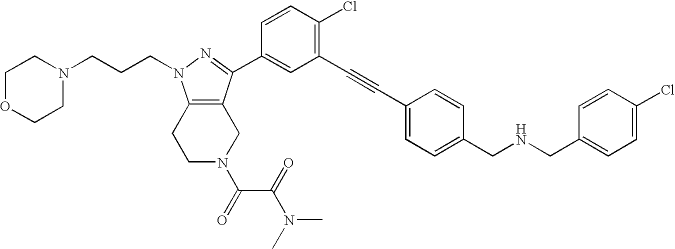

### Example 152

### 2-[3-(4-Chloro-3-{[4-({[(4-chlorophenyl)methyl]amino}methyl)phenyl]ethynyl}phenyl)-1-(3-morpholin-4-ylpropyl)-1,4,6,7-tetrahydro-5H-pyrazolo[4,3-c]pyridin-5-yl]-N,N-dimethyl-2-oxoacetamide

The compound was purified using preparatory HPLC (Method F; HCl salt). MS (ESI): mass calcd. for C₃₉H₄₂Cl₂N₆O₃, 712.27; m/z found, 713.3 [M+H]⁺. ¹H NMR: 7.89 (m, 1H), 7.70-7.65 (m, 3H), 7.63-7.58 (m, 3H), 7.57-7.54 (m, 2H), 7.52-7.47 (m, 2H), 4.87-4.85 (m, 1H), 4.32 (d, J=15, 4H), 4.28 (t, J=6.6, 2H), 4.07-4.01 (m, 3H), 3.86-3.74 (m, 4H), 3.52 (d, J=13, 2H), 3.31-3.24 (m, 2H), 3.17 (dt, J=12, 3.6, 2H), 3.07 (d, J=11, 5H), 3.01-2.92 (m, 4H), 2.44-2.35 (m, 2H).

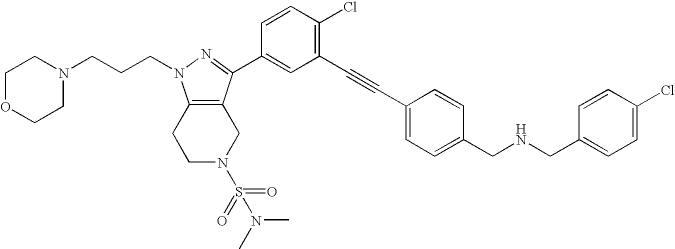

### Example 153

### 3-(4-Chloro-3-{[4-({[(4-chlorophenyl)methyl]amino}methyl)phenyl]ethynyl}phenyl)-N,N-dimethyl-1-(3-morpholin-4-ylpropyl)-1,4,6,7-tetrahydro-5H-pyrazolo[4,3-c]pyridine-5-sulfonamide

The compound was purified using preparatory HPLC (Method F; HCl salt). MS (ESI): mass calcd. for C₃₇H₄₂Cl₂N₆O₃S, 720.24; m/z found, 721.3 [M+H]⁺. ¹H NMR (DMSO): 7.90 (d, J=1.7, 1H), 7.71-7.62 (m, 7H), 7.60 (dd, J=8.4, 1.7, 1H), 7.51 (d, J=8.3, 2H), 4.49 (s, 2H), 4.22-4.13 (m, 6H), 3.93 (d, J=11, 2H), 3.88-3.84 (m, 3H), 3.58 (t, J=5.3, 2H), 3.41 (d, J=12, 2H), 3.19-3.11 (m, 2H), 3.11-2.99 (m, 2H), 2.90-2.86 (m, 2H), 2.80-2.73 (m, 6H), 2.34-2.27 (m, 2H).

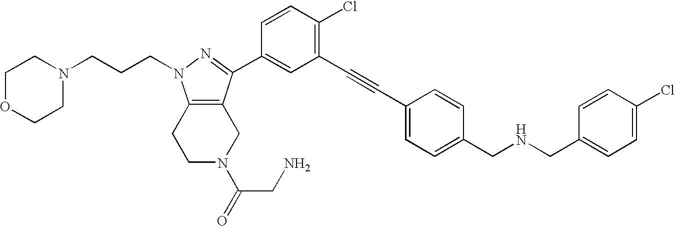

### Example 154

### 2-[3-(4-Chloro-3-{[4-({[(4-chlorophenyl)methyl]amino}methyl)phenyl]ethynyl}phenyl)-1-(3-morpholin-4-ylpropyl)-1,4,6,7-tetrahydro-5H-pyrazolo[4,3-c]pyridin-5-yl]-2-oxoethanamine

The compound was purified using preparatory HPLC (Method E). Collected fractions were concentrated and the residue was treated with HCl (1.25 N in MeOH; 3 mL) in CH₂Cl₂ (3 mL). After 18 h, the mixture was concentrated to give the compound as the HCl salt. MS (ESI): mass calcd. for C₃₇H₄₀Cl₂N₆O₂, 670.26; m/z found, 671.3 [M+H]⁺. ¹H NMR: 7.99-7.89 (m, 1H), 7.72-7.64 (m, 3H), 7.63-7.59 (m, 3H), 7.57-7.52 (m, 2H), 7.51-7.48 (m, 2H), 4.86 (s, 2H), 4.70 (d, J=18, 1H), 4.36-4.25 (m, 6H), 4.17 (s, 1.3H), 4.11 (s, 0.7H), 4.04 (d, J=13, 3H), 3.89-3.75 (m, 4H), 3.54 (d, J=12, 2H), 3.31-3.24 (m, 3H), 3.18 (t, J=11, 2H), 3.05 (s, 1.3H), 2.92 (s, 0.7H), 2.41 (d, J=1.0, 2H).

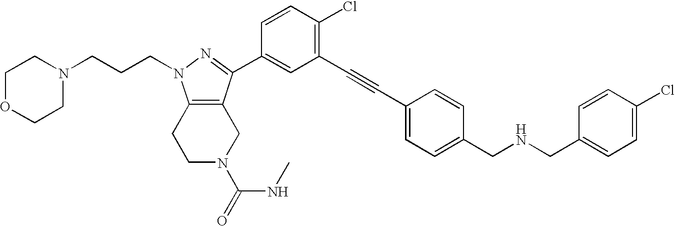

### Example 155

### 3-(4-Chloro-3-{[4-({[(4-chlorophenyl)methyl]amino}methyl)phenyl]ethynyl}phenyl)-N-methyl-1-(3-morpholin-4-ylpropyl)-1,4,6,7-tetrahydro-5H-pyrazolo[4,3-c]pyridine-5-carboxamide

The compound was purified using preparatory HPLC (Method F; HCl salt). MS (ESI): mass calcd. for C₃₇H₄₀Cl₂N₆O₂, 670.26; m/z found, 671.3 [M+H]⁺. ¹H NMR: 7.84 (d, J=2.1, 1H), 7.61-7.54 (m, 3H), 7.53-7.48 (m, 3H), 7.47-7.45 (m, 2H), 7.40-7.36 (m, 2H), 4.94 (s, 1H), 4.54 (s, 2H), 4.24-4.18 (m, 6H), 3.93 (dd, J=13, 3.2, 2H), 3.75-3.67 (m, 4H), 3.42 (d, J=13, 2H), 3.20-3.16 (m, 2H), 3.07 (dt, J=12, 3.6, 2H), 2.77 (t, J=5.6, 2H), 2.66 (s, 3H), 2.35-2.25 (m, 2H).

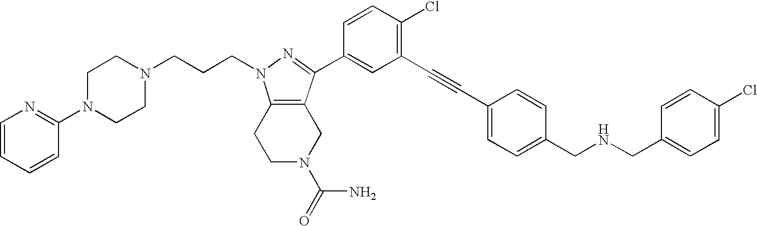

### Example 156

### 3-(4-Chloro-3-{[4-({[(4-chlorophenyl)methyl]amino}methyl)phenyl]ethynyl}phenyl)-1-[3-(4-pyridin-2-ylpiperazin-1-yl)propyl]-1,4,6,7-tetrahydro-5H-pyrazolo[4,3-c]pyridine-5-carboxamide

This compound was prepared according to the methods described in Example 150, following sequentially Steps A, B, C, E, F, and then D with the appropriate substituent changes. The compound was purified using preparatory HPLC (Method B; TFA salt). MS (ESI): mass calcd. for C₄₁H₄₂Cl₂N₈O, 732.29; m/z found, 733.3 [M+H]⁺. ¹H NMR: 8.17 (t, J=7.7, 1H), 8.08 (d, J=5.8, 1H), 7.96-7.93 (m, 1H), 7.69 (d, J=7.9, 3H), 7.64-7.57 (m, 3H), 7.57-7.53 (m, 2H), 7.53-7.45 (m, 3H), 7.17 (t, J=6.5, 1H), 4.73 (s, 2H), 4.53-4.36 (m, 2H), 4.31 (d, J=12.2, 6H), 4.01-3.55 (m, 8H), 3.43-3.34 (m, 3H), 2.98-2.91 (m, 2H), 2.52-2.43 (m, 2H), 1.43-1.25 (m, 2H).

The compounds in Examples 157-165 were prepared using methods analogous to those described for Example 156.

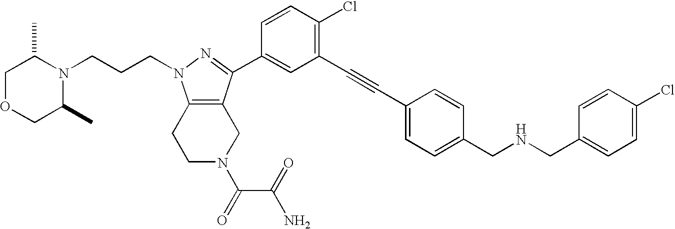

### Example 157

### 2-(3-{4-Chloro-3-[(4-{[(4-chlorobenzyl)amino]methyl}phenyl)ethynyl]phenyl}-1-{3-[(3S,5S)-3,5-dimethylmorpholin-4-yl]propyl}-1,4,6,7-tetrahydro-5H-pyrazolo[4,3-c]pyridin-5-yl)-2-oxoacetamide

The compound was purified using preparatory HPLC (Method B; HCl salt). MS (ESI): mass calcd. for C₃₉H₄₂Cl₂N₆O₃, 712.27; m/z found, 714.2 [M+H]⁺. ¹H NMR: 7.93-7.83 (m, 1H), 7.71-7.67 (m, 3H), 7.60 (t, J=9.3, 3H), 7.55-7.49 (m, 4H), 4.93-4.90 (m, 1H), 4.84-4.81 (m, 1H), 4.31 (d, J=13.2, 4H), 4.28-4.24 (m, 3H), 4.06-3.93 (m, 3H), 3.89 (q, J=13.0, 2H), 3.77-3.69 (m, 1H), 3.69-3.60 (m, 1H), 3.62-3.42 (m, 2H), 3.38-3.35 (m, 2H), 3.22-3.08 (m, 1H), 3.09-2.87 (m, 2H), 2.50-2.36 (m, 1H), 2.31-2.19 (m, 1H), 1.42 (d, J=6.5, 3H), 1.30 (d, J=6.1, 3H).

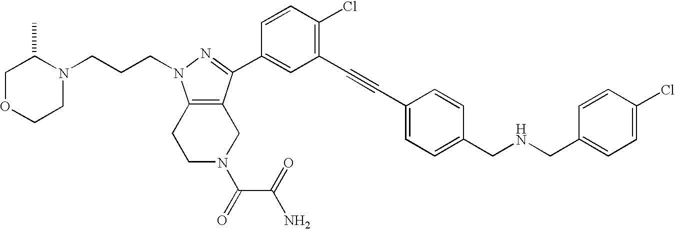

### Example 158; 2-[3-(4-Chloro-3-{[4-({[(4-chlorophenyl)methyl]amino}methyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide.

The compound was purified using preparatory HPLC (Method B; HCl salt). MS (ESI): mass calcd. for C₃₈H₄₀Cl₂N₆O₃, 698.25; m/z found, 699.2 [M+H]⁺. ¹H NMR: 7.90-7.89 (m, 0.7H), 7.84-7.82 (m, 0.3H), 7.70-7.66 (m, 3H), 7.61-7.55 (m, 3H), 7.53-7.45 (m, 4H), 4.81 (s, 1H), 4.31 (s, 2H), 4.28 (s, 2H), 4.27-4.22 (m, 2H), 4.05 (d, J=13, 1H), 4.02-3.94 (m, 3H), 3.87-3.68 (m, 2H), 3.58-3.39 (m, 4H), 3.35-3.32 (m, 1H), 3.26-3.13 (m, 3H), 3.02-2.85 (m, 2H), 2.47-2.35 (m, 1H), 2.33-2.20 (m, 1H), 1.45-1.22 (m, 4H).

### Alternative Synthesis of 2-[3-(4-Chloro-3-{[4-({[(4-chlorophenyl)methyl]amino]methyl)phenyl]ethynyl]phenyl)-1-[3-[(3S)-3-methylmorpholin-4-yl]propyl]-1,4,6,7-tetrahydro-5H-pyrazolo[4,3-c]pyridin-5-yl]-2-oxoacetamide.

A. 4-(3-Chloro-propyl}-(3S)-methyl-morpholine. A solution of (3S)-methyl-morpholine (1 g, 9.9 mmol, 1.0 equiv) and 1-bromo-3-chloro-propane (3.1 g, 19.8 mmol, 2.0 equiv) in THF (5 mL) was treated with NaH (60%, 2 equiv) in two portions. The resulting slurry was heated at 65° C. for 18 h. Slowly, the reaction was quenched with ice water (20 mL). During the addition, excess bubbling occurred. The resulting mixture was stirred for 16 h and then was extracted with EtOAc (3×15 mL). The combined organic layers were extracted with 1 N HCl (40 mL). The aqueous layer was cooled in an ice bath and basified slowly to pH ˜9-10 with NaOH pellets. The aqueous layer was extracted with EtOAc (3×15 mL). The combined organic layers were dried and concentrated to give the title compound as a clear oil (1.3 g, 74%).

B. 3-(4-Chloro-3-iodo-phenyl)-4,5,6,7-tetrahydro-1H-pyrazolo[4,3-c]pyridine. A suspension of 3-(4-chloro-3-iodo-phenyl)-1,4,6,7-tetrahydro-pyrazolo[4,3-c]pyridine-5-carboxylic acid tert-butyl ester (260 g, 0.57 mol, 1.0 equiv) in CH₂Cl₂ (750 mL) was treated with TFA (250 mL) over 20 min. The resulting solution was stirred at rt for 16 h. The mixture was diluted with water (2 L) and then basified with satd. aq. NaOH to a pH>12. The mixture was stirred for 3 h. The white precipitate was collected by filtration, washed with water and dried in a vacuum oven to provide the title compound (205 g, 0.57 mol, 100%), which was used in the next reaction without further purification.

C. 2-[3-(4-Chloro-3-iodo-phenyl)-1,4,6,7-tetrahydro-pyrazolo[4,3-c]pyridin-5-yl]-2-oxo-acetamide. A suspension of CDI (110.5 g, 0.68 mol, 1.2 equiv) in DMF (1.5 L) was treated with oxalamic acid (60.7 g, 0.68 mol, 1.2 equiv). After 3 h at rt, 3-(4-chloro-3-iodo-phenyl)-4,5,6,7-tetrahydro-1H-pyrazolo[4,3-c]pyridine (205 g, 0.57 mol, 1.0 equiv) was added as solid over 10 min. After 20 min, water (2.5 L) was added slowly over 2 h and the mixture was stirred at rt for 16 h. The resulting white precipitate was collected by filtration, washed with water and dried in a vacuum oven to provide the title compound (243 g, 0.56 mol, 100%), which was used in the next reaction without further purification.

D. 2-[3-(4-Chloro-3-iodo-phenyl)-1-[3-((3S)-methyl-morpholin-4-yl)-propyl]-1,4,6,7-tetrahydro-pyrazolo[4,3-c]pyridin-5-yl]-2-oxo-acetamide. A solution of 2-[3-(4-chloro-3-iodo-phenyl)-1,4,6,7-tetrahydro-pyrazolo[4,3-c]pyridin-5-yl]-2-oxo-acetamide (190 g, 0.44 mol, 1.0 equiv) in DMF (1.8 L) was treated with Cs₂CO₃ (180 g, 0.55 mol, 1.25 equiv) followed by 4-(3-chloro-propyl)-(3S)-methyl-morpholine (86 g, 0.48 mol, 1.1 equiv). The reaction mixture was stirred via mechanical stirrer at 50° C. under N₂ for 12 h and then cooled to rt. Water (3.5 L) was added slowly over 30 min. The mixture was stirred at rt for 16 h and the resulting white solid was collected by filtration, washed with water and dried in a vacuum oven. The crude compound was triturated from hot EtOH (−1.5 L) to provide the title compound (176 g, 0.31 mol, 70%).

E. 2-[3-(4-Chloro-3-{[4-(ff(4-chlorophenyl)methyl]amino]methyl)phenyl]ethynyl}phenyl)-1-{3-[(3S)-3-methylmorpholin-4-yl]propyl-1,4,6,7-tetrahydro-5H-pyrazolo[4,3-c]pyridin-5-yl-2-oxoacetamide. To a 5 L flask equipped with a mechanical stirrer and an internal thermocouple, was added 2-{3-(4-chloro-3-iodo-phenyl)-1-[3-((3S)-methyl-morpholin-4-yl)-propyl]-1,4,6,7-tetrahydro-pyrazolo[4,3-c]pyridin-5-yl}-2-oxo-acetamide (120 g, 0.21 mol, 1.0 equiv), (4-chloro-benzyl)-(4-ethynyl-benzyl)-amine (56.3 g, 0.22 mol, 1.0 equiv), DMF (1.5 L) and Et₃N (120 mL, 0.84 mol, 4.0 equiv) sequentially. A stream of N₂ was bubbled into the solution for 15 min. A mixture of Pd(PPh₃)₂Cl₂ (0.37 g, 0.5 mmol, 0.0025 equiv) and CuI (0.2 g, 1.0 mmol, 0.005 equiv) was added under N₂. The solution was degassed with N₂ for another 10 min. The reaction solution was stirred at 50° C. for 16 h. The reaction solution was cooled to rt and water (2 L) was added with stirring. The liquid layer was decanted away from an oily precipitate, which was then partitioned between EtOAc (2 L) and 2:1 water/satd. aq. NaHCO₃ (1.5 L). The organic layer was dried and concentrated to provide the crude material as a foamy yellow solid (145 g, ˜85% purity by HPLC). The crude material was purified (SiO₂; 2 N NH₃ in MeOH/CH₂Cl₂) to provide the title compound (87 g, 59%, >98% purity).

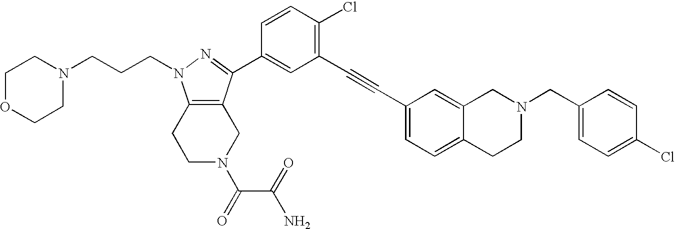

### Example 159

### 2-{3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridin-5-yl}-2-oxoacetamide

The compound was purified using preparatory HPLC (Method B; HCl salt). MS (ESI): mass calcd. for C₃₉H₄₀Cl₂N₆O₃, 710.25; m/z found, 711.2 [M+H]⁺. ¹H NMR: 7.80-7.69 (m, 1H), 7.59-7.54 (m, 1H), 7.54-7.38 (m, 6H), 7.35 (s, 1H), 7.25 (d, J=7.9, 1H), 4.70 (s, 2H), 4.43 (s, 2H), 4.36 (d, J=7.3, 2H), 4.20-4.09 (m, 2H), 4.01-3.77 (m, 4H), 3.72 (s, 1H), 3.64 (t, J=12.0, 2H), 3.40 (d, J=12.8, 3H), 3.18-3.12 (m, 5H), 3.11-2.96 (m, 3H), 2.87 (s, 1H), 2.83-2.78 (m, 1H), 2.34-2.14 (m, 2H).

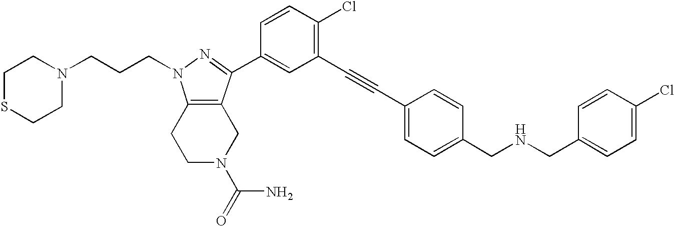

### Example 160

### 3-(4-Chloro-3-{[4-({[(4-chlorophenyl)methyl]amino}methyl)phenyl]ethynyl}phenyl)-1-(3-thiomorpholin-4-ylpropyl)-1,4,6,7-tetrahydro-5H-pyrazolo[4,3-c]pyridine-5-carboxamide

The compound was purified using preparatory HPLC (Method B; HCl salt). MS (ESI): mass calcd. for C₃₆H₃₈Cl₂N₆OS, 672.22; m/z found, 673.3 [M+H]⁺. ¹H NMR: 7.92-7.90 (m, 1H), 7.70-7.65 (m, 3H), 7.60-7.55 (m, 3H), 7.53-7.47 (m, 4H), 4.93-4.90 (m, 1H), 4.66 (s, 2H), 4.29 (d, J=16.2, 4H), 4.24 (t, J=6.4, 2H), 3.85-3.74 (m, 4H), 3.35-3.33 (m, 1H), 3.28-3.17 (m, 5H), 3.06 (t, J=13.3, 2H), 2.87-2.81 (m, 4H), 2.38-2.31 (m, 2H).

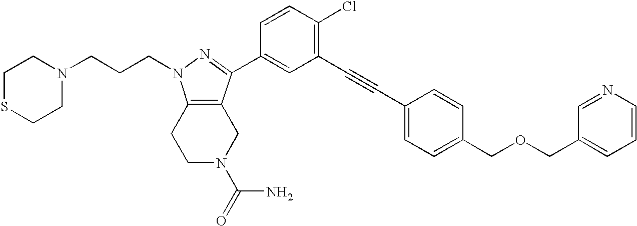

### Example 161

### 3-{4-Chloro-3-[(4-{[(pyridin-3-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide

The compound was purified using preparatory HPLC (Method B; HCl salt). MS (ESI): mass calcd. for C₃₅H₃₇ClN₆O₂S, 640.24; m/z found, 641.2 [M+H]⁺. ¹H NMR: 8.89 (s, 1H), 8.81 (d, J=5.4, 1H), 8.67 (d, J=7.9, 1H), 8.11 (dd, J=7.8, 6.0, 1H), 7.91 (s, 1H), 7.65 (d, J=8.3, 1H), 7.62-7.56 (m, 3H), 7.51-7.47 (m, 2H), 4.85 (s, 2H), 4.77 (s, 2H), 4.67 (s, 2H), 4.27-4.23 (m, 2H), 3.87-3.74 (m, 4H), 3.28-3.17 (m, 5H), 3.14-3.03 (m, 2H), 2.89-2.79 (m, 4H), 2.36 (d, J=1.2, 2H), 1.29-1.28 (m, 1H).

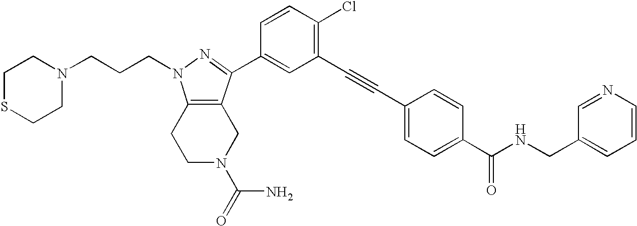

### Example 162

### 3-{4-Chloro-3-[(4-{[(pyridin-3-ylmethyl)amino]carbonyl}phenyl)ethynyl]phenyl}-1-(3-thiomorpholin-4-ylpropyl)-1,4,6,7-tetrahydro-5H-pyrazolo[4,3-c]pyridine-5-carboxamide

The compound was purified using preparatory HPLC (Method B; HCl salt). MS (ESI): mass calcd. for C₃₅H₃₆ClN₇O₂S, 653.23; m/z found, 654.2 [M+H]⁺. ¹H NMR: 8.92 (s, 1H), 8.80 (d, J=5.2, 1H), 8.68 (d, J=8.0, 1H), 8.11 (dd, J=7.9, 5.8, 1H), 7.99-7.90 (m, 3H), 7.73-7.65 (m, 3H), 7.59 (d, J=8.3, 1H), 4.83-4.74 (m, 3H), 4.70 (s, 2H), 4.30-4.20 (m, 2H), 3.82 (d, J=10.3, 3H), 3.35 (s, 4H), 3.28-3.17 (m, 4H), 3.17-3.06 (m, 2H), 2.95-2.73 (m, 3H), 2.43-2.30 (m, 2H).

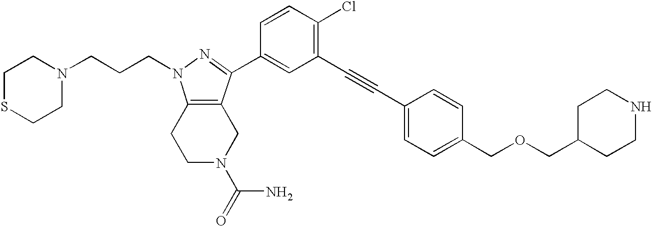

### Example 163

### 3-{4-Chloro-3-[(4-{[(piperidin-4-ylmethyl)oxy]methyl}phenyl)ethynyl]phenyl}-1-(3-thiomorpholin-4-ylpropyl)-1,4,6,7-tetrahydro-5H-pyrazolo[4,3-c]pyridine-5-carboxamide

The compound was purified using preparatory HPLC (Method B; HCl salt). MS (ESI): mass calcd. for C₃₅H₄₃ClN₆O₂S, 646.29; m/z found, 647.3 [M+H]⁺. ¹H NMR: 7.91 (s, 1H), 7.65 (dd, J=8.4, 2.1, 1H), 7.57 (dd, J=9.6, 8.4, 3H), 7.40 (d, J=8.1, 2H), 4.93 (s, 1H), 4.66 (s, 2H), 4.56 (s, 2H), 4.24 (t, J=6.5, 2H), 3.85-3.75 (m, 4H), 3.47-3.36 (m, 5H), 3.28-3.17 (m, 5H), 3.14-3.03 (m, 2H), 3.00 (t, J=11.8, 2H), 2.88-2.79 (m, 4H), 2.42-2.28 (m, 2H), 2.03-1.96 (m, 3H), 1.57-1.48 (m, 2H).

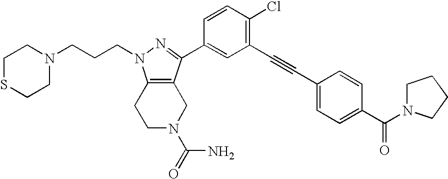

### Example 164

### 3-(4-Chloro-3-{[4-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide

The compound was purified using preparatory HPLC (Method B; HCl salt). MS (ESI): mass calcd. for C₃₃H₃₇ClN₆O₂S, 616.24; m/z found, 617.2 [M+H]⁺. ¹H NMR (DMSO): 7.93 (d, J=1.6, 1H), 7.70 (dd, J=8.5, 1.7, 1H), 7.67 (d, J=8.5, 3H), 7.60 (d, J=8.1, 2H), 4.57 (s, 2H), 4.14 (t, J=6.8, 2H), 3.71 (d, J=8.4, 2H), 3.64 (t, J=5.4, 2H), 3.48 (t, J=6.7, 2H), 3.44-3.31 (m, 4H), 3.20-3.09 (m, 6H), 2.81 (d, J=13.1, 2H), 2.73 (t, J=4.5, 2H), 2.31-2.18 (m, 2H), 1.88 (td, J=13.0, 6.4, 2H), 1.85-1.79 (m, 2H).

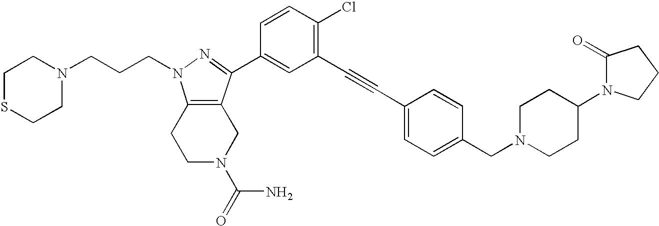

### Example 165

### 3-{4-Chloro-3-[(4-{[4-(2-oxopyrrolidin-1-yl)piperidin-1-yl]methyl}phenyl)ethynyl]phenyl}-1-(3-thiomorpholin-4-ylpropyl)-1,4,6,7-tetrahydro-5H-pyrazolo[4,3-c]pyridine-5-carboxamide

The compound was purified using preparatory HPLC (Method B; HCl salt). MS (ESI): mass calcd. for C₃₈H₄₆ClN₇O₂S, 699.31; m/z found, 700.3 [M+H]⁺. ¹H NMR: 7.95 (d, J=2.0, 1H), 7.72 (d, J=8.2, 2H), 7.69 (dd, J=8.4, 2.1, 1H), 7.65 (d, J=8.2, 2H), 7.60 (d, J=8.4, 1H), 4.69 (s, 2H), 4.39 (s, 2H), 4.26 (t, J=6.5, 2H), 4.21-4.08 (m, 1H), 3.86-3.80 (m, 4H), 3.60 (d, J=12.6, 2H), 3.46 (t, J=7.0, 2H), 3.30-3.05 (m, 9H), 2.91-2.80 (m, 4H), 2.39 (dd, J=15.2, 6.9, 4H), 2.21-2.10 (m, 2H), 2.10-2.01 (m, 2H), 2.00-1.92 (m, 2H), 1.30 (s, 1H).

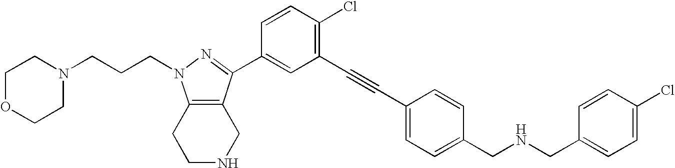

### Example 166

### 1-[4-({2-Chloro-5-[1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine

The title compound was prepared according to the methods describe for Example 150, but the reaction described in Step G proceeded for 1 h. The compound was purified using preparatory HPLC (Method B; TFA salt). MS (ESI): mass calcd. for C₃₅H₃₇Cl₂N₅O, 613.24; m/z found, 614.2 [M+H]⁺. ¹H NMR: 7.88 (s, 1H), 7.66 (d, J=8.1, 2H), 7.60-7.58 (m, 2H), 7.56 (d, J=8.2, 2H), 7.52-7.47 (m, 4H), 4.94-4.91 (m, 2H), 4.48 (s, 2H), 4.33-4.23 (m, 6H), 4.12-3.91 (m, 2H), 3.87-3.67 (m, 2H), 3.61 (t, J=6.1, 2H), 3.57-3.42 (m, 2H), 3.34 (s, 1H), 3.25 (dd, J=9.4, 6.7, 2H), 3.16 (t, J=6.0, 3H), 2.41-2.34 (m, 2H).

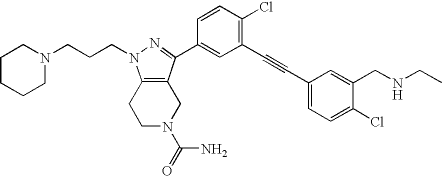

### Example 167

### 3-[4-Chloro-3-({4-chloro-3-[(ethylamino)methyl]phenyl}ethynyl)phenyl]-1-(3-piperidin-1-ylpropyl)-1,4,6,7-tetrahydro-5H-pyrazolo[4,3-c]pyridine-5-carboxamide

A. 3-(4-Chloro-3-iodo-phenyl)-1-(3-piperidin-1-yl-prolyl)-1,4,6,7-tetrahydro-pyrazolo[4,3-c]pyridine-5-carboxylic acid amide. This compound was prepared according to the methods described in Example 150, following sequentially Steps A, B, C, E, and then F, with the appropriate substituent changes. MS (ESI): mass calcd. for C₂₁H₂₇CIIN₅O, 527.09; m/z found, 528.1 [M+H]⁺.

B. 3-(4-Chloro-3-trimethylsilanylethynyl-phenyl)-1-(3-piperidin-1-yl-propyl)-1,4,6,7-tetrahydro-pyrazolo[4,3-c]pyridine-5-carboxylic acid amide. This compound was prepared according to the methods described for Intermediate 1, Step E. MS (ESI): mass calcd. for C₂₆H₃₆ClN₅OSi, 497.24; m/z found, 498.2 [M+H]⁺.

C. 3-[4-Chloro-3-({4-chloro-3-[(ethylamino)methyl]phenyl}ethynyl)phenyl]-1-(3-piperidin-1-ylpropyl)-1,4,6,7-tetrahydro-5H-pyrazolo[4,3-c]oyridine-5-carboxamide. This compound was prepared according to the methods described for Intermediate 1, Step E, substituting (2-chloro-5-iodo-benzyl)-ethyl-amine for (trimethylsilyl)acetylene, DMF instead of THF, DBU instead of TEA, and adding 2.8 eq. H₂O. The compound was purified using preparatory HPLC (Method C; HCl salt). MS (ESI): mass calcd. for C₃₂H₃₈Cl₂N₆O, 592.25; m/z found, 593.1 [M+H]⁺. ¹H NMR: 8.02-7.78 (m, 2H), 7.70-7.55 (m, 4H), 4.76-4.67 (m, 2H), 4.46-4.37 (m, 2H), 4.30-4.22 (m, 2H), 3.94-3.79 (m, 2H), 3.54 (d, J=9.2, 2H), 3.29-3.13 (m, 4H), 3.03-2.85 (m, 4H), 2.45-2.30 (m, 2H), 1.92 (d, J=12.6, 2H), 1.80 (d, J=11.5, 4H), 1.59-1.46 (m, 1H), 1.43-1.38 (m, 4H), 1.31-1.26 (m, 1H).

The compounds in Examples 168-169 were prepared using methods analogous to those described in Example 167, with the appropriate substituent changes.

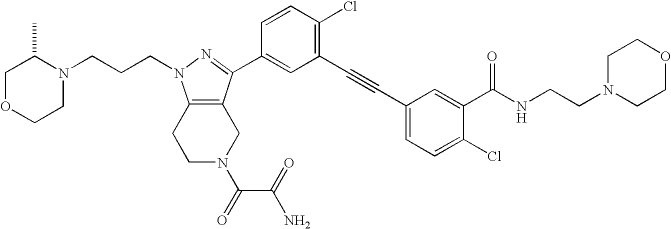

### Example 168

### 5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(2-morpholin-4-ylethyl)benzamide

The compound was purified using preparatory HPLC (Method B; TFA salt). MS (ESI): mass calcd. for C₃₇H₄₃Cl₂N₇O₅, 735.27; m/z found, 736.3 [M+H]⁺. ¹H NMR: 7.88-7.79 (m, 1H), 7.74 (d, J=1.9, 1H), 7.66 (dd, J=8.4, 2.1, 1H), 7.62 (dd, J=8.3, 2.0, 1H), 7.58-7.52 (m, 2H), 4.83-4.76 (m, 1H), 4.25-4.19 (m, 2H), 4.17-4.00 (m, 3H), 3.99-3.91 (m, 4H), 3.87-3.70 (m, 5H), 3.69-3.59 (m, 2H), 3.59-3.49 (m, 3H), 3.49-3.42 (m, 3H), 3.28-3.12 (m, 5H), 2.96-2.92 (m, 1.3H), 2,89-2.85 (m, 0.7H), 2.47-2.32 (m, 1H), 2.32-2.18 (m, 1H), 1.39 (s, 1H), 1.27 (d, J=5.6, 2H).

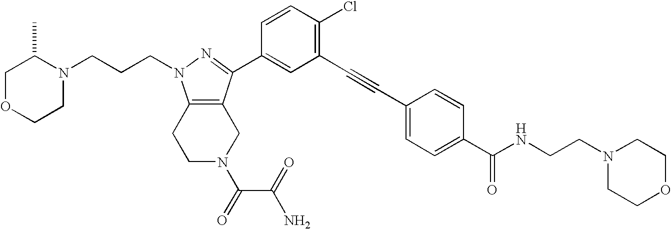

### Example 169

### 4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(2-morpholin-4-ylethyl)benzamide

The compound was purified using preparatory HPLC (Method D; formic acid salt). MS (ESI): mass calcd. for C₃₇H₄₄ClN₇O₅, 701.31; m/z found, 702.2 [M+H]⁺.

¹H NMR: 7.92-7.80 (m, 3H), 7.66 (d, J=8.4, 2H), 7.63 (dd, J=8.4, 2.1, 1H), 7.56-7.54 (m, 1H), 4.88-4.85 (m, 2H), 4.79 (s, 2H), 4.14 (t, J=5.9, 2H), 3.95 (td, J=19.4, 5.8, 2H), 3.85-3.79 (m, 1H), 3.73 (dd, J=11.8, 2.7, 1H), 3.69-3.64 (m, 1H), 3.51 (t, J=6.6, 2H), 3.36-3.32 (m, 2H), 3.08-3.04 (m, 2H), 3.02-2.97 (m, 2H), 2.97-2.86 (m, 2H), 2.72 (s, 4H), 2.62-2.49 (m, 2H), 2.23-2.13 (m, 1H), 2.12-2.05 (m, 1H), 2.03-1.97 (m, 2H), 1.04 (d, J=6.4, 3H).

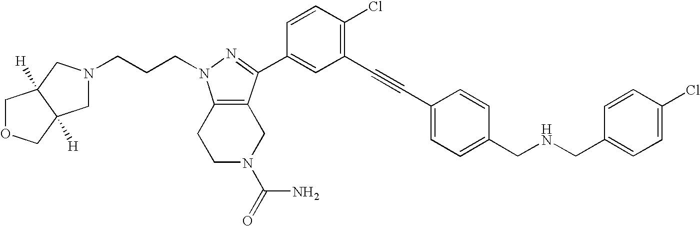

### Example 170

### 3-[4-Chloro-3-[(4-{[(4-chlorobenzyl)amino]methyl]phenyl)ethynyl]phenyl}-1-{3-[(3aR,6aS)-tetrahydro-1H-furo[3,4-c]pyrrol-5(3H)-yl]propyl}-1,4,6,7-tetrahydro-5H-pyrazolo[4,3-c]pyridine-5-carboxamide

A. (4-[5-[5-Carbamoyl—(3-hydroxy-propyl)-4,5,6,7-tetrahydro-1 H-pyrazolo[4,3-c]pyridin-3-yl]-2-chloro-phenylethynyl]-benzyl)-(4-chloro-benzyl)-carbamic acid tert-butyl ester. This compound was prepared according to the methods described in Example 150, following sequentially Steps A, E, F, D, B, and then C, with the appropriate substituent changes. MS (ESI): mass calcd. for C₃₇H₃₉Cl₂N₅O₄, 687.24; m/z found, 688.2 [M+H]⁺.

B. 3-[4-Chloro-3-[(4-{[(4-chlorobenzyl)amino]methyl]phenyl)ethynyl]phenyl}-1-{3-[(3aR,6aS)-tetrahydro-1H-furo[3,4-c]pyrrol-5(3H)-yl]propyl-1,4,6,7-tetrahydro-5H-pyrazolo[4,3-c]pyridine-5-carboxamide. A solution of the carbamate above in 2.5:1 CH₂Cl₂:TFA (7 mL) was stirred at rt for 2 h. The reaction was quenched with satd. aq. NaHCO₃ and extracted with CH₂Cl₂ (2×). The combined organic layers were dried and concentrated, and the residue was purified using preparatory HPLC (Method E; HCl salt). MS (ESI): mass calcd. for C₃₈H₄₀Cl₂N₆O₂, 682.26; m/z found, 683.2 [M+H]⁺. ¹H NMR: 7.91 (d, J=1.7, 1H), 7.71-7.62 (m, 3H), 7.60-7.55 (m, 3H), 7.56-7.45 (m, 4H), 4.67 (s, 2H), 4.29 (d, J=10.3, 4H), 4.26-4.21 (m, 2H), 4.00-3.88 (m, 1H), 3.86-3.77 (m, 4H), 3.72-3.65 (m, 1H), 3.59 (d, J=11.6, 1H), 3.53 (dd, J=9.6, 5.3, 1H), 3.34 (s, 3H), 3.25-3.15 (m, 2H), 3.14-3.05 (m, 1H), 2.86 (t, J=5.6, 2H), 2.77 (dd, J=10.6, 8.8, 1H), 2.40-2.23 (m, 2H).

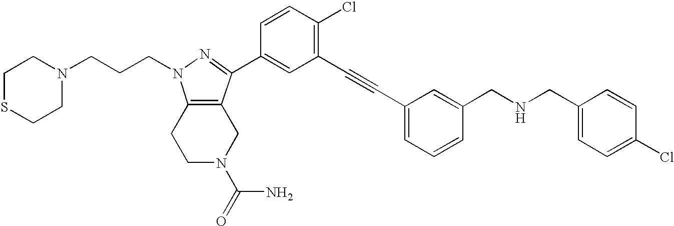

### Example 171

### 3-(4-Chloro-3-{[3-({[(4-chlorophenyl)methyl]amino}methyl)phenyl]ethynyl}phenyl)-1-(3-thiomorpholin-4-ylpropyl)-1,4,6,7-tetrahydro-5H-pyrazolo[4,3-c]pyridine-5-carboxamide

A. 3-[4-Chloro-3-(3-formyl-phenylethynyl)-phenyl]-1-(3-thiomorpholin-4-yl-propyl)-1,4,6,7-tetrahydro-prazolo[4,3-c]pyridine-5-carboxylic acid amide. This compound was prepared according to the methods described in Example 150, following sequentially Steps A, B, C, E, and F, and then Example 167, Step C, with the appropriate substituent changes. MS (ESI): mass calcd. for C₂₉H₃₀ClN₅O₂S, 547.18; m/z found, 548.2 [M+H]⁺.

B. 3-(4-Chloro-3-{[3-(ff(4-chlorophenyl)methyl]amino]methyl)phenyl]ethynyl]phenyl)-1-(3-thiomorpholin-4-ylpropyl)-1,4,6,7-tetrahydro-5H-pyrazolo[4,3-c]pyridine-5-carboxamide. A solution of the aldehyde above (0.194 mmol), 4-chlorobenzylamine (0.388 mmol), and AcOH (16 μL) in CH₂Cl₂ (2 mL) was stirred for 30 min, and then treated with NaBH(OAc)₃ (0.388 mmol). After 23 h, the mixture was diluted with satd. aq. NaHCO₃ and extracted CH₂Cl₂ (3×). The combined organic layers were dried and concentrated. The residue was purified using preparatory HPLC (Method F; HCl salt). MS (ESI): mass calcd. for C₃₆H₃₈Cl₂N₆OS, 672.22; m/z found, 673.3 [M+H]⁺. ¹H NMR: 7.84 (s, 1H), 7.70 (s, 1H), 7.58 (d, J=7.8, 2H), 7.53-7.35 (m, 7H), 4.60 (s, 2H), 4.21 (d, J=13.9, 4H), 4.16 (t, J=6.0, 2H), 3.77-3.68 (m, 4H), 3.21-3.07 (m, 5H), 3.03 (t, J=13.1, 2H), 2.79 (s, 2H), 2.74 (d, J=13.8, 2H), 2.32-2.25 (m, 2H).

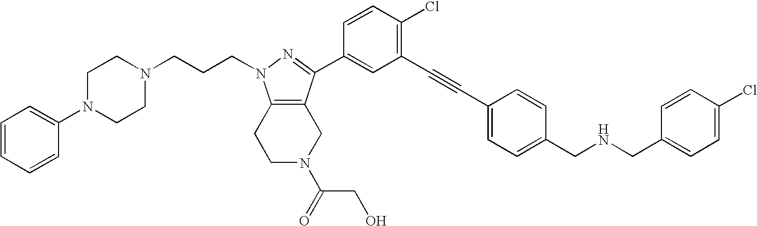

### Example 172

### 2-{3-(4-Chloro-3-{[4-({[(4-chlorophenyl)methyl]amino}methyl)phenyl]ethynyl}phenyl)-1-[3-(4-phenylpiperazin-1-yl)propyl]-1,4,6,7-tetrahydro-5H-pyrazolo[4,3-c]pyridin-5-yl}-2-oxoethanol

The title compound was prepared using methods analogous to those described for Example 150, following sequentially Steps A, D, B, C, E, F, and then G. MS (ESI): mass calcd. for C₄₃H₄₄Cl₂N₆O₂, 746.29; m/z found, 748.2 [M+H]⁺. ¹H NMR: 7.65-7.51 (m, 4H), 7.41-7.39 (m, 1H), 7.25 (dd, J=8.6, 7.4, 1H), 6.92 (d, J=7.9, 1H), 4.34-4.11 (m, 4H), 3.56-2.92 (m, 24H).

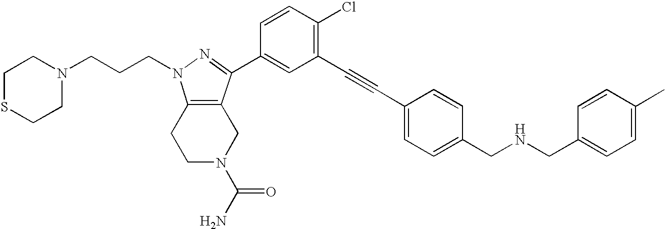

### Example 173

### 3-(4-Chloro-3-{[4-({[(4-methylphenyl)methyl]amino}methyl)phenyl]ethynyl}phenyl)-1-(3-thiomorpholin-4-ylpropyl)-1,4,6,7-tetrahydro-5H-pyrazolo[4,3-c]pyridine-5-carboxamide

A. 3-[4-Chloro-3-(4-formyl-phenylethynyl)-phenyl]-1-(3-thiomorpholin-4-yl-propyl)-1,4,6,7-tetrahydro-pyrazolo[4,3-c]pyridine-5-carboxylic acid amide. The title compound was prepared using methods analogous to those described for Example 151. MS (ESI): mass calcd. for C₂₉H₃₀ClN₅O₂S, 548.10; m/z found 549.3 [M+H]⁺.

B. 3-(4-Chloro-3-{[4-(ff(4-methylphenyl)methyl]amino]methyl)phenyl]ethynyl]phenyl)-1-(3-thiomorpholin-4-ylpropyl)-1,4,6,7-tetrahydro-5H-pyrazolo[4,3-c]pyridine-5-carboxamide. The title compound was prepared using methods analogous to those described for Example 171, Step B. The compound was purified by Preparative HPLC Method B. MS (ESI): mass calcd. for C₃₇H₄₁ClN₆OS, 652.29; m/z found, 653.3 [M+H]⁺. ¹H NMR: 7.91 (d, J=2.1, 1H), 7.70-7.65 (m, 3H), 7.59 (dd, J=11.1, 8.4, 3H), 7.41 (d, J=8.1, 2H), 7.30 (d, J=7.9, 2H), 4.66 (s, 2H), 4.30-4.21 (m, 6H), 3.87-3.75 (m, 4H), 3.30-3.08 (m, 6H), 2.92-2.78 (m, 4H), 2.40-2.34 (m, 4H).

The compounds in Examples 174-176 were prepared using methods analogous to those described for Example 173.

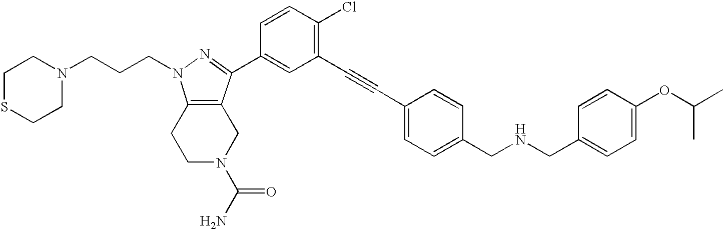

### Example 174

### 3-{4-Chloro-3-[(4-{[({4-[(1-methylethyl)oxy]phenyl}methyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide

MS (ESI): mass calcd. for C₃₉H₄₅ClN₆O₂S, 696.34; m/z found, 697.3 [M+H]⁺. ¹H NMR: 7.92 (d, J=2.1, 1H), 7.71-7.67 (m, 3H), 7.62-7.54 (m, 3H), 7.47-7.35 (m, 2H), 7.01-6.98 (m, 2H), 4.72-4.62 (m, 3H), 4.29-4.19 (m, 5H), 3.87-3.70 (m, 3H), 3.30-3.15 (m, 4H), 3.14-2.75 (m, 6H), 2.38-2.32 (m, 2H), 1.33 (d, J=6.0, 6H).

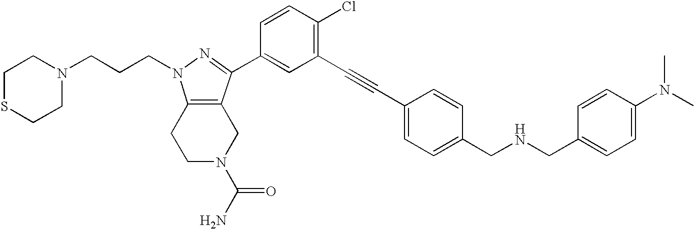

### Example 175

### 3-[4-Chloro-3-({4-[({[4-(dimethylamino)phenyl]methyl}amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide

MS (ESI): mass calcd. for C₃₈H₄₄ClN₇OS, 681.33; m/z found, 682.3 [M+H]⁺. ¹H NMR: 7.92 (d, J=2.1, 1H), 7.71-7.65 (m, 3H), 7.61-7.53 (m, 3H), 7.41-7.37 (m, 1H), 6.98 (d, J=8.8, 2H), 4.66 (s, 2H), 4.30-4.18 (m, 6H), 3.89-3.77 (m, 4H), 3.30-3.19 (m, 4H), 3.08-3.03 (m, 7H), 2.91-2.78 (m, 4H), 2.38-2.32 (m, 2H), 2.05 (s, 2H).

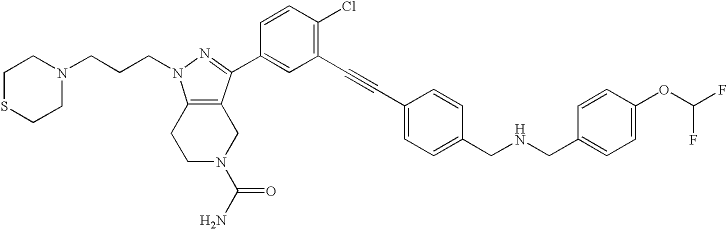

### Example 176

### 3-{4-Chloro-3-[(4-{[({4-[(difluoromethyl)oxy]phenyl(methyl)amino]methyl(phenyl)ethynyl]phenyl}-1-(3-thiomorpholin-4-ylpropyl)-1,4,6,7-tetrahydro-5H-pyrazolo[4,3-c]pyridine-5-carboxamide

MS (ESI): mass calcd. for C₃₇H₃₉ClF₂N₆O₂S, 704.27; m/z found, 705.2 [M+H]⁺. ¹H NMR: 7.93 (d, J=2.1, 1H), 7.71-7.67 (m, 3H), 7.62-7.54 (m, 5H), 7.26 (d, J=8.6, 2H), 4.66 (s, 2H), 4.31 (d, J=11.1, 4H), 4.24 (t, J=6.5, 2H), 3.79 (t, J=5.7, 3H), 3.29-2.76 (m, 10H), 2.39-2.31 (m, 2H).

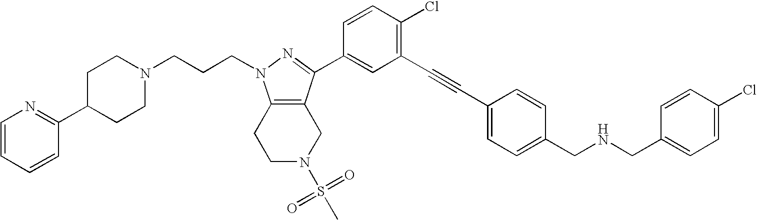

### Example 177

### 1-{4-[(2-Chloro-5-{5-(methylsulfonyl)-1-[3-(4-pyridin-2-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine

A. (4-Chlorobenzyl)-(4-ethynylbenzyl)-amine. Acetic acid (0.95 g, 15.7 mmol) was added at once to a solution of 4-chlorobenzylamine (2.2 g, 15.4 mmol) and 4-ethynylbenzaldehyde (1.0 g, 7.7 mmol) in CH₂Cl₂ (40 mL). The resulting slurry was stirred for 30 min and sodium triacetoxyborohydride (2.5 g, 11.6 mmol) was added portionwise. After 3 days, the reaction mixture was diluted with satd. aq. NaHCO₃ and extracted with CH₂Cl₂ (×3). The combined organic extracts were dried (Na₂SO₄) and concentrated to give a yellow oil. Purification by silica gel chromatography (0 to 30% EtOAc/hexanes) afforded the title compound as a colorless oil (1.7 g, 85%). HPLC: Rₜ=4.70. MS (ESI): mass calcd. for C₁₆H₁₄ClN, 256.7; m/z found, 256.1 [M+H]⁺. ¹H NMR (CDCl₃): 7.46 (d, J=8.2, 2H), 7.31-7.27 (m, 4H), 3.78 (s, 2H), 3.75 (s, 2H), 3.06 (s, 1H), 1.58 (br s, 1H).

B. 1-[4-[(2-Chloro-5-[5-(methylsulfonyl)-1-{3-(4-pyridin-2-ylpiperidin-1-yl)propyl]-4,5,6,7-tetrahydro-1H-pyrazolo[4,3-c]pyridin-3-ylphenyl)ethynyl]phenyl]-N-[(4-chlorophenyl)methyl]methanamine. The title compound was prepared using methods analogous to those described for intermediate 1, substituting 2-(4 piperidinyl)pyridine for morpholine in Step D, and (4-chlorobenzyl)-(4-ethynylbenzyl)-amine for TMSA in Step E. HPLC: Rₜ=4.39. HPLC: Rₜ=4.39. MS (ESI): mass calcd. for C₄₂H₄₄Cl₂N₆O₂S, 766.3; m/z found, 767.4 [M+H]⁺. ¹H NMR (CDCl₃): 8.55-8.52 (m, 1H), 7.82 (d, J=2.0, 1H), 7.62 (dt, J=7.7, 1.8, 1H), 7.56 (d, J=8.1, 2H), 7.49 (dd, J=8.4, 2.1, 1H), 7.45 (d, J=8.4, 1H), 7.38 (d, J=8.1, 2H), 7.30-7.27 (m, 4H), 7.17 (d, J=7.9, 1H), 7.12 (ddd, J=7.5, 4.9, 1.0, 1H), 4.54 (s, 2H), 4.12 (t, J=6.7, 2H), 3.81 (s, 2H), 3.76 (s, 2H), 3.66 (t, J=5.7, 2H), 3.00-2.91 (m, 5H), 2.89 (s, 3H), 2.74-2.65 (m, 1H), 2.33 (d, J=6.7, 2H), 2.14-2.02 (m, 4H), 1.98-1.91 (m, 2H), 1.87-1.75 (m, 2H).

The compounds in Examples 178-183 were prepared using methods analogous to those described for Example 177, substituting the appropriate amine for 2-(4-piperidinyl)pyridine in Step B.

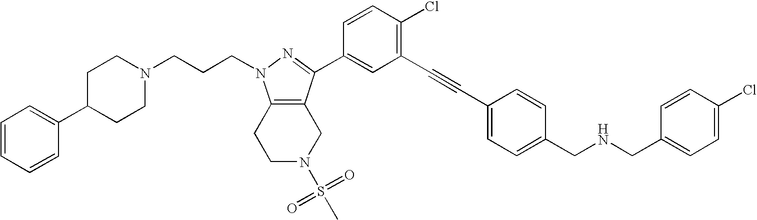

### Example 178

### 1-{4-[(2-Chloro-5-{5-(methylsulfonyl)-1-[3-(4-phenylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine

HPLC: Rₜ=4.77. MS (ESI): mass calcd. for C₄₃H₄₅Cl₂N₅O₂S, 765.3; m/z found, 766.3 [M+H]⁺. ¹H NMR (CDCl₃): 7.81 (d, J=2.0, 1H), 7.58-7.54 (m, 2H), 7.50 (dd, J=8.4, 2.1, 1H), 7.45 (d, J=8.4, 1H), 7.35-7.30 (m, 4H), 7.29 (d, J=2.7, 4H), 7.24-7.19 (m, 3H), 4.54 (s, 2H), 4.12 (t, J=6.8, 2H), 3.81 (s, 2H), 3.77 (s, 2H), 3.66 (t, J=5.7, 2H), 3.00-2.90 (m, 4H), 2.89 (s, 3H), 2.54-2.45 (m, 1H), 2.35 (t, J=6.9, 2H), 2.15-2.08 (m, 2H), 2.07-2.00 (m, 2H), 1.88-1.82 (m, 2H), 1.79-1.68 (m, 3H).

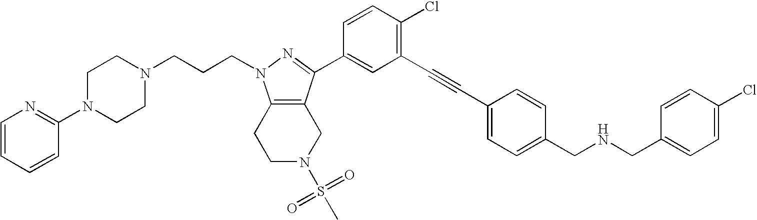

### Example 179

### 1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine

HPLC: Rₜ=4.35. MS (ESI): mass calcd. for C₄₁H₄₃Cl₂N₇O₂S, 767.3; m/z found, 768.3 [M+H]⁺. ¹H NMR (CDCl₃): 8.20-8.17 (m, 1H), 7.81 (d, J=1.9, 1H), 7.59-7.55 (m, 2H), 7.57-7.44 (m, 3H), 7.36-7.33 (m, 2H), 7.30-7.28 (m, 4H), 6.66-6.61 (m, 2H), 4.54 (s, 2H), 4.13 (t, J=6.8, 2H), 3.81 (s, 2H), 3.77 (s, 2H), 3.65 (t, J=5.8, 2H), 3.55-3.51 (m, 4H), 2.93-2.91 (m, 2H), 2.90 (s, 3H), 2.54-2.51 (m, 4H), 2.37 (t, J=6.7, 2H), 2.16-2.09 (m, 2H), 1.64 (bs, 1H).

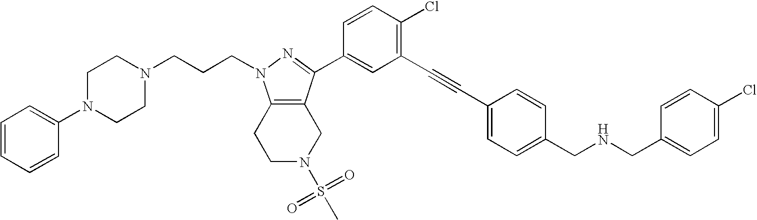

### Example 180

### 1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine

HPLC: Rₜ=4.76. MS (ESI): mass calcd. for C₄₂H₄₄Cl₂N₆O₂S, 766.3; m/z found, 767.3 [M+H]⁺. ¹H NMR (CDCl₃): 7.81 (d, J=1.9, 1H), 7.58-7.55 (m, 2H), 7.49 (dd, J=8.4, 2.0, 1H), 7.45 (d, J=8.4, 1H), 7.36-7.32 (m, 2H), 7.30-7.25 (m, 6H), 6.94-6.90 (m, 2H), 6.88-6.83 (m, 1H), 4.53 (s, 2H), 4.12 (t, J=6.8, 2H), 3.80 (s, 2H), 3.76 (s, 2H), 3.64 (t, J=5.8, 2H), 3.20-3.17 (m, 4H), 2.92-2.89 (m, 2H), 2.89 (s, 3H), 2.58-2.55 (m, 4H), 2.37 (t, J=6.8, 2H), 2.15-2.08 (m, 2H), 1.87-1.83 (m, 1H).

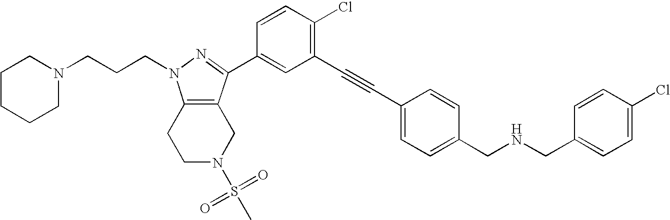

### Example 181

### 1-[4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine

HPLC: Rₜ=4.60. MS (ESI): mass calcd. for C₃₇H₄₁Cl₂N₅O₂S, 689.2; m/z found, 690.3 [M+H]⁺. ¹H NMR (CDCl₃): 7.80 (d, J=1.9, 1H), 7.58-7.55 (m, 2H), 7.49 (dd, J=8.4, 2.0, 1H), 7.45 (d, J=8.4, 1H), 7.35-7.32 (m, 2H), 7.30-7.28 (m, 4H), 4.53 (s, 2H), 4.08 (t, J=6.8, 2H), 3.81 (2, 2H), 3.77 (s, 2H), 3.65 (t, J=5.8, 2H), 2.93-2.90 (m, 2H), 2.89 (s, 3H), 2.36-2.30 (bm, 4H), 2.25 (t, J=6.8, 2H), 2.10-2.02 (m, 2H), 1.83 (bs, 1H), 1.59-1.53 (m, 4H), 1.47-1.40 (m, 2H).

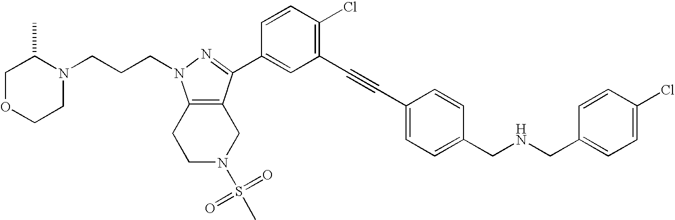

### Example 182

### 1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine

HPLC: Rₜ=4.53. MS (ESI): mass calcd. for C₃₇H₄₁Cl₂N₅O₃S, 705.2; m/z found, 706.3 [M+H]⁺. ¹H NMR (CDCl₃): 7.80-7.79 (m, 1H), 7.56 (d, J=7.9, 2H), 7.48 (dd, J=8.4, 2.0, 1H), 7.45 (d, J=8.4, 1H), 7.34 (d, J=8.0, 2H), 7.31-7.27 (m, 4H), 4.53 (s, 2H), 4.12-4.01 (m, 2H), 3.81 (s, 2H), 3.81-3.79 (m, 1H), 3.79 (s, 2H), 3.68-3.61 (m, 4H), 3.24 (dd, J=11.0, 8.8, 1H), 2.95-2.85 (m, 2H), 2.90 (s, 3H), 2.80-2.70 (m, 2H), 2.44-2.36 (m, 1H), 2.30-2.20 (m, 2H), 2.12-1.98 (m, 2H), 1.77 (bs, 1H), 0.91 (d, J=6.3, 3H).

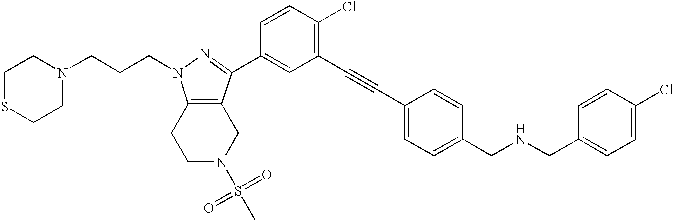

### Example 183

### 1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine

HPLC: Rₜ=4.57. MS (ESI): mass calcd. for C₃₆H₃₉Cl₂N₅O₂S, 707.2; m/z found, 708.3 [M+H]⁺. ¹H NMR (CDCl₃): 7.80 (d, J=1.8, 1H), 7.56 (d, J=8.1, 2H), 7.48 (dd, J=8.4, 2.0, 1H), 7.45 (d, J=8.3, 1H), 7.34 (d, J=8.1, 2H), 7.30-7.28 (m, 6H), 4.53 (s, 2H), 4.07 (t, J=6.8, 2H), 3.81 (s, 2H), 3.79 (s, 2H), 3.64 (t, J=5.8, 2H), 3.28 (s, 3H), 2.89-2.85 (m, 2H), 2.68-2.63 (m, 8H), 2.34 (t, J=6.8, 2H), 2.09-2.01 (m, 2H).

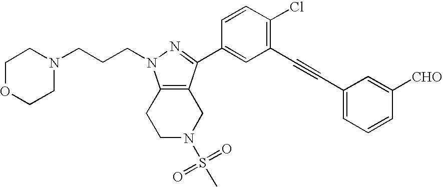

### Example 184

### 3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)benzaldehyde

A solution of DMF (15 mL) and distilled water (1.6 mL) was degassed with nitrogen for 1 h and transferred via syringe to a degassed flask containing 3-(4-chloro-3-iodophenyl)-5-(methylsulfonyl)-1-(3-morpholin-4-ylpropyl)-4,5,6,7-tetrahydro-1H-pyrazolo[4,3-c]pyridine (2.3 g, 4.0 mmol), Pd(PPh₃)₂Cl₂ (281 mg, 0.4 mmol), and CuI (76 mg, 0.4 mmol). The mixture was treated with 3-trimethylsilanylethynyl-benzaldehyde (1.2 g, 6.0 mmol) and DBU (1.8 g, 12 mmol), and the resulting mixture was stirred at rt under N₂ for 18 h. The mixture was concentrated under high vacuum, and the crude residue was diluted with satd. aq. NaCl and extracted with CH₂Cl₂. The combined organic layers were dried and concentrated to give a brown oil. Purification by silica gel chromatography (0 to 3% MeOH/CH₂Cl₂) afforded the title compound as a beige foam (1.7 g, 76%). HPLC: Rₜ=4.86. MS (ESI): mass calcd. for C₂₉H₃₁ClN₄O₄S, 566.2; m/z found, 567.1 [M+H]⁺. ¹H NMR (CDCl₃): 10.00 (s, 1H), 8.10-8.09 (m, 1H), 7.90-7.87 (m, 1H), 7.86-7.84 (m, 1H), 7.82 (d, J=1.9, 1H), 7.57 (t, J=7.7, 1H), 7.52 (d, J=2.1, 1H), 7.48 (d, J=8.4, 1H), 4.55 (s, 2H), 4.11 (t, J=6.8, 2H), 3.70 (t, J=4.6, 4H), 3.67 (t, J=5.8, 2H), 2.93 (s, 3H), 2.93-2.89 (m, 2H), 2.44-2.40 (m, 4H), 2.34 (t, J=6.8, 2H), 2.12-2.06 (m, 2H).

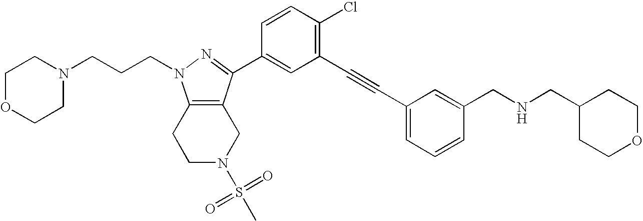

### Example 185

### 1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydro-2H-pyran-4-ylmethyl)methanamine

To a solution of 3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)benzaldehyde (77 mg, 0.14 mmol) in CH₂Cl₂ (1.4 mL) were added 1-tetrahydro-2H-pyran-4-ylmethanamine (31 mg, 0.27 mmol) and ACOH (4 μL). The resulting slurry was stirred at rt for 30 min, then was treated with Na(OAc)₃BH (57 mg, 0.27 mmol) and stirred for an additional 16 h. The mixture was diluted with satd. aq. NaHCO₃ and extracted with CH₂Cl₂ (3×). The combined organic layers were dried and concentrated to afford a yellow oil. Purification of the residue (SiO₂; 0-3% 2 M NH₃ in MeOH/CH₂Cl₂) afforded the title compound as a white powder (72 mg, 79%). HPLC: Rₜ=4.24. MS (ESI): mass calcd. for C₃₅H₄₄ClN₅O₄S, 665.3; m/z found, 666.2 [M+H]⁺. ¹H NMR (CDCl₃): 7.80 (d, J=1.8, 1H), 7.68 (s, 1H), 7.51-7.47 (m, 2H), 7.46 (d, J=8.4, 1H), 7.33 (d, J=4.9, 1H), 4.54 (s, 2H), 4.10 (t, J=6.8, 2H), 4.02-3.95 (m, 3H), 3.70 (t, J=4.6, 4H), 3.65 (t, J=5.8, 2H), 3.43-3.35 (m, 3H), 2.91 (s, 3H), 2.91-2.88 (m, 2H), 2.43-2.38 (m, 4H), 2.32 (t, J=6.8, 2H), 2.11-2.04 (m, 2H), 1.77-1.61 (m, 4H), 1.37-1.25 (m, 4H).

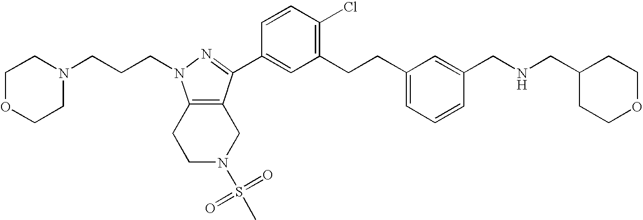

### Example 186

### 1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(tetrahydro-2H-pyran-4-ylmethyl)methanamine

The title compound was prepared from 1-[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydro-2H-pyran-4-ylmethyl)methanamine using a method analogous to described for Example 2. HPLC: Rₜ=4.24. MS (ESI): mass calcd. for C₃₅H₄₈ClN₅O₄S, 669.3; m/z found, 670.3 [M+H]⁺. ¹H NMR (CDCl₃): 7.51 (d, J=2.1, 1H), 7.40 (d, J=8.3, 1H), 7.32 (dd, J=8.3, 2.1, 1H), 7.27 (t, J=7.5, 1H), 7.21-7.14 (m, 3H), 4.47 (s, 2H), 4.10 (t, J=6.8, 2H), 3.97 (dd, J=10.8, 3.5, 2H), 3.77 (s, 2H), 3.70 (t, J=4.6, 4H), 3.65 (t, J=5.8, 2H), 3.39 (dt, J=11.8, 1.8, 2H), 3.08-3.03 (m, 2H), 2.97-2.92 (m, 2H), 2.89 (s, 3H), 2.91-2.88 (m, 2H), 2.53 (d, J=6.5, 2H), 2.43-2.39 (m, 4H), 2.33 (t, J=6.8, 2H), 2.11-2.04 (m, 2H), 1.76-1.63 (m, 4H), 1.36-1.25 (m, 2H).

The compounds in Examples 187-188 were prepared using methods analogous to those described for Example 185.

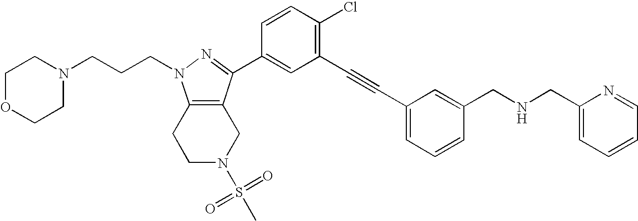

### Example 187

### 1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine

HPLC: Rₜ=4.27. MS (ESI): mass calcd. for C₃₅H₃₉ClN₆O₃S, 658.3; m/z found, 659.2 [M+H]⁺. ¹H NMR (CDCl₃): 8.57 (d, J=4.4, 1H), 7.79 (d, J=1.9, 1H), 7.65 (dt, J=7.7, 1.8, 1H), 7.61 (s, 1H), 7.56-7.44 (m, 3H), 7.40-7.36 (m, 1H), 7.34 (t, J=7.4, 2H), 7.19-7.15 (m, 1H), 4.53 (s, 2H), 4.10 (t, J=6.8, 2H), 3.94 (s, 2H), 3.87 (s, 2H), 3.70 (t, J=4.6, 4H), 3.65 (t, J=5.8, 2H), 2.90 (s, 3H), 2.90-2.87 (m, 2H), 2.42-2.38 (m, 4H), 2.32 (t, J=6.8, 2H), 2.11-2.05 (m, 3H).

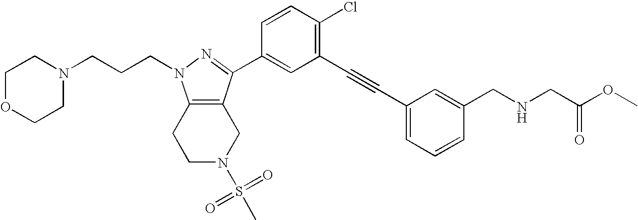

### Example 188

### Methyl N-{[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate

HPLC: Rₜ=4.42. MS (ESI): mass calcd. for C₃₂H₃₈ClN₅O₅S, 639.2; m/z found, 640.2 [M+H]⁺. ¹H NMR (CDCl₃): 7.79 (d, J=1.8, 1H), 7.58 (s, 1H), 7.52-7.47 (m, 2H), 7.45 (d, J=8.3, 1H), 7.35-7.33 (m, 2H), 4.53 (s, 2H), 4.10 (t, J=6.8, 2H), 3.83 (s, 2H), 3.74 (s, 3H), 3.70 (t, J=4.6, 4H), 3.65 (t, J=5.8, 2H), 3.44 (s, 2H), 2.91 (s, 3H), 2.90-2.87 (m, 2H), 2.43-2.38 (m, 4H), 2.33 (t, J=6.8, 2H), 2.11-2.04 (m, 2H), 1.96 (bs, 1H).

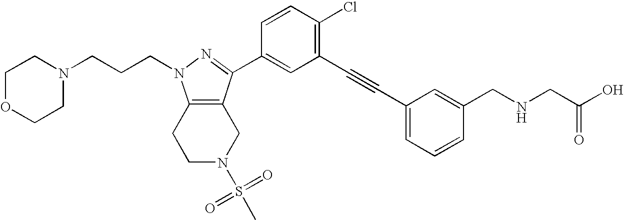

### Example 189

### N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycine

To a solution of methyl N-{[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate (46 mg, 0.07 mmol) in THF (0.3 mL) was added 1 N NaOH (0.3 mL). After 2 h at rt, the mixture was neutralized with 1 N HCl and extracted with 20% IPA/CHCl₃ (3×). The combined organic extracts were dried and concentrated to give the title compound as a beige solid (36 mg, 82%). HPLC: Rₜ=4.17. MS (ESI): mass calcd. for C₃₁H₃₆ClN₅O₅S, 625.2; m/z found, 626.2 [M+H]⁺. ¹H NMR (CDCl₃): 7.74 (d, J=1.6, 1H), 7.64 (bs, 1H), 7.57 (m, 1H), 7.50 (dd, J=8.4, 1.9, 1H), 7.46 (d, J=8.4, 2H), 7.42-7.38 (bm, 1H), 4.51 (s, 2H), 4.09 (t, J=6.8, 4H), 3.73-3.68 (m, 6H), 3.64 (t, J=5.7, 2H), 3.43-3.39 (m, 2H), 2.92 (s, 3H), 2.91-2.87 (m, 2H), 2.45-2.41 (bm, 4H), 2.34 (t, J=7.0, 2H), 2.10-2.03 (m, 2H).

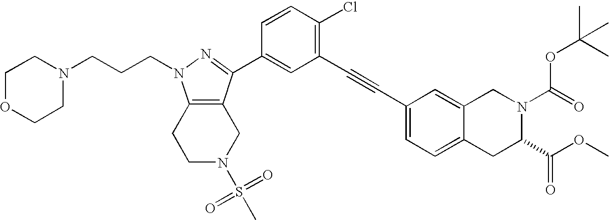

### Example 190

### 2-(1,1-Dimethylethyl) 3-methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate

A. 7-Hydroxy-(3S)-1,2,3,4-tetrahydroisoquinoline-3-carboxylic acid methyl ester. To a cooled (0° C.) slurry of 7-hydroxy-(3S)-1,2,3,4-tetrahydroisoquinoline-3-carboxylic acid (770 mg, 3.6 mmol) in MeOH (13 mL) was added oxalyl chloride (1.8 mL, 18 mmol) dropwise. The resulting mixture was heated at 65° C. for 5 h. After cooling to rt, the reaction was slowly quenched with satd. aq. NaHCO₃, and the solvent was removed in vacuo. The crude residue was dissolved in 20% IPA/CHCl₃ and washed with satd. aq NaHCO₃. The organic layer was dried and concentrated to give the title compound as a white solid (523 mg, 63%). ¹H NMR: 6.92 (d, J=8.3, 1H), 6.59 (dd, J=8.3, 2.5, 1H), 6.47 (d, J=2.4, 1H), 5.48 (s, 1H), 3.97 (d, J=15.9, 1H), 3.91 (d, J=15.9, 1H), 3.75 (s, 3H), 3.67 (dd, J=10.3, 4.7, 1H), 2.97 (dd, J=15.9, 4.7, 1H), 2.82 (dd, J=15.9, 10.3, 1H).

B. 7-Hydroxy-(3S)-3,4-dihydro-1H-isoquinoline-2,3-dicarboxylic acid 2-tert-butyl ester 3-methyl ester. To a solution of the above amine (100 mg, 0.48 mmol) in 2:1 THF:water (2.4 mL) was added di-tert-butyldicarbonate (105 mg, 0.48 mmol) dropwise. After stirring for 18 h, the reaction mixture was diluted with 0.5 N HCl and extracted with EtOAc (3×). The combined organic extracts were dried and concentrated to afford the title compound as a colorless oil (124 mg, 84%). ¹H NMR (CDCl₃): 7.21 (bs, 1H), 6.98 (d, J=8.0, 1H), 6.68 (dt, J=8.1, 2.3, 1H), 6.62-6.60 (m, 1H), 5.11-5.09 (m, 0.4H, minor rotamer), 4.74 (t, J=5.5, 0.6H, major rotamer), 4.68-4.59 (m, 1H), 4.50-4.40 (m, 1H), 3.66 (s, 1.8H, major rotamer), 3.59 (s, 1.2H, minor rotamer), 3.18-3.02 (m, 2H), 1.52 (s, 3.6H, minor rotamer), 1.46 (s, 5.4H, major rotamer).

C. 7-Trifluoromethanesulfonyloxy-(3S)-3,4-dihydro-1H-isoquinoline-2,3-dicarboxylic acid 2-tert-butyl ester 3-methyl ester. To an ice-cooled solution of the above phenol (157 mg, 0.51 mmol) in CH₂Cl₂ (2.6 mL) was added pyridine (161 mg, 2.04 mmol) and trifluoromethanesulfonic anhydride (288 mg, 1.02 mmol). The mixture was warmed to rt and stirred at rt for 4 h. The reaction mixture was diluted with 0.5 N HCl and extracted with CH₂Cl₂ (3×). The combined organic extracts were dried and concentrated to give the title compound as an orange oil (220 mg, 98%). ¹H NMR (CDCl₃): 7.23 (d, J=8.4, 1H), 7.11 (d, J=8.5, 1H), 7.06 (d, J=9.8, 1H), 5.20 (dd, J=6.2, 2.5, 0.4H, minor rotamer), 4.84 (t, J=5.2, 0.6H, major rotamer), 4.58-4.44 (m, 1H), 3.65 (s, 1.2H, minor rotamer), 3.64 (s, 1.8H, major rotamer), 3.30 (dd, J=16.2, 2.2, 0.6H, major rotamer), 3.20-3.16 (m, 1H), 3.16-3.12 (m, 0.4H, minor rotamer), 1.53 (s, 5.4H, major rotamer), 1.47 (s, 3.6H, minor rotamer).

D. 2-(1,1-Dimethylethyl) 3-methyl (3S)-7-([2-chloro-5-[5-(methylsulfonyl)-1-(3-morpholin-4-ylpropyl)-4,5,6,7-tetrahydro-1H-pyrazolo[4,3-c]pyridin-3-yl]-phenylethynyl)-3,4-dihydroisoquinoline-2,3(1H)-dicarboxylate. The title compound was prepared using methods analogous to those described in Example 1, Step B, substituting DMF for THF and 7-trifluoromethanesulfonyloxy-(3S)-3,4-dihydro-1H-isoquinoline-2,3-dicarboxylic acid 2-tert-butyl ester 3-methyl ester for 5-iodoindole. HPLC: Rₜ=5.38. MS (ESI): mass calcd. for C₃₈H₄₆ClN₅O₇S, 751.3; m/z found, 752.1 [M+H]⁺. ¹H NMR (CDCl₃): 7.78-7.76 (m, 1H), 7.51-7.47 (m, 1H), 7.44 (d, J=8.4, 1H), 7.39 (d, J=8.7, 2H), 7.16 (d, J=7.7, 1H), 5.17 (dd, J=6.0, 2.8, 0.6H, major rotamer), 4.82 (t, J=5.2, 0.4H, minor rotamer), 4.74 (d, J=16.9, 1H), 4.56-4.47 (m, 1H), 4.53 (s, 2H), 4.10 (t, J=6.8, 2H), 3.69 (t, J=4.6, 4H), 3.67-3.63 (m, 2H), 3.65 (s, 1.2H, minor rotamer), 3.63 (s, 1.8H, major rotamer), 3.28 (dd, J=16.1, 2.6, 0.6H, major rotamer), 3.21-3.14 (m, 0.4H, minor rotamer), 3.19-3.17 (m, 1H), 2.90 (s, 3H), 2.90-2.86 (m, 2H), 2.42-2.38 (m, 4H), 2.32 (t, J=6.8, 2H), 2.11-2.03 (m, 2H), 1.54 (s, 5H, major rotamer), 1.50 (s, 4H, minor rotamer).

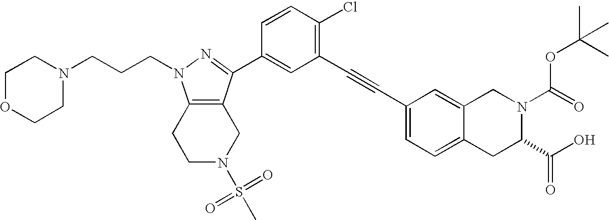

### Example 191

### (3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid

To a solution of 2-(1,1-dimethylethyl) 3-methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate (379 mg, 0.50 mmol) in THF (2 mL) was added 1 N NaOH (2 mL). The reaction mixture was stirred at rt for 4 h and then neutralized with 1 N HCl. Following extraction with CH₂Cl₂ (2×) and 20% IPA/CHCl₃, the combined organic extracts were dried and concentrated to afford the title compound as a white solid (356 mg, 96%). HPLC: Rₜ=5.14. MS (ESI): mass calcd. for C₃₇H₄₄ClN₅O₇S, 737.3; m/z found, 738.1 [M+H]⁺.

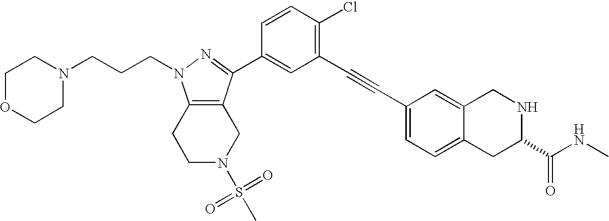

### Example 192

### (3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-methyl-1,2,3,4-tetrahydroisoquinoline-3-carboxamide

To a solution of (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid (68 mg, 0.09 mmol), HOBt (14 mg, 0.10 mmol), and EDC (19 mg, 0.10 mmol) in CH₂Cl₂(1.0 mL) was added methylamine (2.0 M in THF; 70 μL, 0.14 mmol) and iPr₂NEt (24 mg, 0.18 mmol). The reaction mixture was stirred at rt for 16 h, diluted with satd. aq. NaHCO₃, and extracted with CH₂Cl₂ (3×). The combined organic extracts were dried and concentrated to give a brown oil. Purification (SiO₂: 0 to 3% 2 M NH₃ in MeOH/CH₂Cl₂) afforded a white foam, which was dissolved in 2:1 CH₂Cl₂:TFA (1.0 mL) and stirred at rt for 1 h. The mixture was diluted with satd. aq. NaHCO₃ and extracted with CH₂Cl₂ (3×). The combined organic extracts were dried and concentrated to give the title compound as a white solid (34 mg, 56%). HPLC: Rₜ=4.12. MS (ESI): mass calcd. for C₃₃H₃₉ClN₆O₄S, 650.2; m/z found, 651.1 [M+H]⁺. ¹H NMR (CDCl₃): 7.78 (d, J=1.9, 1H), 7.49 (dd, J=8.4, 2.0, 1H), 7.45 (d, J=8.4, 1H), 7.40 (dd, J=7.8, 1.4, 1H), 7.30 (s, 1H), 7.23-7.20 (m, 1H), 7.17 (d, J=7.9, 1H), 4.53 (s, 2H), 4.10 (t, J=6.8, 2H), 4.00 (s, 2H), 3.79 (t, J=4.6, 4H), 3.65 (t, J=5.8, 2H), 3.55 (dd, J=10.5, 5.1, 1H), 3.27 (dd, J=16.9, 5.1, 1H), 2.91 (s, 3H), 2.91-2.88 (m, 3H), 2.86 (d, J=5.0, 3H), 2.85-2.80 (m, 1H), 2.43-2.39 (m, 4H), 2.32 (t, J=6.8, 2H), 2.11-2.05 (m, 2H), 1.80 (bs, 1H).

The compounds in Examples 193-194 were prepared using methods analogous to those described for Examples 190-192, substituting the appropriate amine for methylamine.

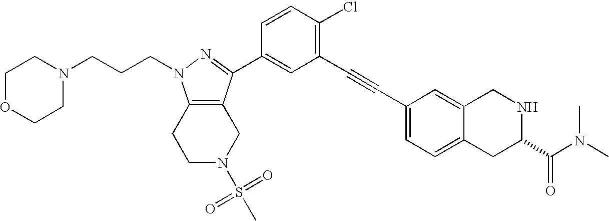

### Example 193

### (3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N,N-dimethyl-1,2,3,4-tetrahydroisoquinoline-3-carboxamide

HPLC: Rₜ=4.17. MS (ESI): mass calcd. for C₃₄H₄₁ClN₆O₄S, 664.3; m/z found, 665.1 [M+H]⁺. ¹H NMR (CDCl₃): 7.78 (d, J=1.9, 1H), 7.49 (dd, J=8.4, 2.0, 1H), 7.45 (d, J=8.4, 1H), 7.37 (dd, J=7.9, 1.4, 1H), 7.30 (s, 1H), 7.10 (d, J=7.9, 1H), 4.54 (s, 2H), 4.13-4.08 (m, 4H), 3.94 (dd, J=10.8, 3.8, 1H), 3.70 (t, J=4.6, 4H), 3.65 (t, J=5.8, 2H), 3.14 (s, 3H), 3.02 (s, 3H), 2.99-2.94 (m, 1H), 2.91 (s, 3H), 2.91-2.88 (m, 2H), 2.85-2.79 (m, 1H), 2.43-2.39 (m, 4H), 2.32 (t, J=6.8, 2H), 2.11-2.04 (m, 2H), 1.94 (bs, 1H).

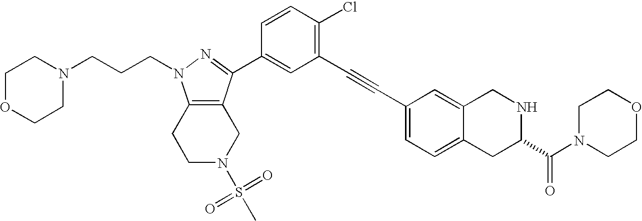

### Example 194

### (3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(morpholin-4-ylcarbonyl)-1,2,3,4-tetrahydroisoquinoline

HPLC: Rₜ=4.10. MS (ESI): mass calcd. for C₃₆H₄₃ClN₆O₅S, 706.3; m/z found, 707.2 [M+H]⁺. ¹H NMR (CDCl₃): 7.78 (d, J=1.9, 1H), 7.49 (dd, J=8.4, 2.0, 1H), 7.45 (d, J=8.4, 1H), 7.38 (dd, J=7.9, 1.4, 1H), 7.29 (s, 1H), 7.11 (d, J=7.9, 1H), 4.53 (s, 2H), 4.12-4.08 (m, 4H), 3.89 (dd, J=10.9, 4.2, 1H), 3.76-3.72 (m, 4H), 3.71-3.68 (m, 7H), 3.65 (t, J=5.8, 2H), 3.56-3.52 (m, 1H), 3.07-2.99 (m, 1H), 2.90 (s, 3H), 2.90-2.87 (m, 2H), 2.79 (dd, J=16.9, 3.6, 1H), 2.43-2.38 (m, 4H), 2.32 (t, J=6.8, 2H), 2.11-2.04 (m, 2H), 1.90 (bs, 1H).

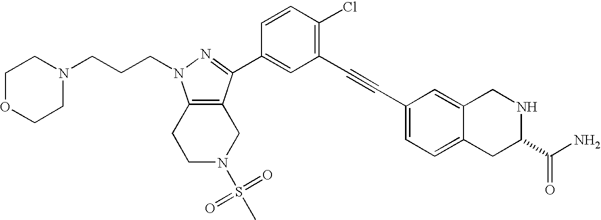

### Example 195

### (3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide

The title compound was prepared using methods analogous to those described for Examples 190-192, substituting ammonium chloride for methylamine, DMF for THF, and N-methylmorpholine for iPr₂NEt in Example 192. HPLC: Rₜ=4.09. MS (ESI): mass calcd. for C₃₂H₃₇ClN₆O₄S, 636.2; m/z found, 637.1 [M+H]⁺. ¹H NMR (CDCl₃): 7.78 (d, J=1.9, 1H), 7.49 (dd, J=8.4, 2.0, 1H), 7.45 (d, J=8.4, 1H), 7.40 (dd, J=7.8, 1.4, 1H), 7.30 (s, 1H), 7.16 (d, J=7.9, 1H), 7.07 (bs, 1H), 5.73 (bs, 1H), 4.53 (s, 2H), 4.10 (t, J=6.8, 2H), 4.03 (s, 2H), 3.70 (t, J=4.6, 4H), 3.65 (t, J=5.8, 2H), 3.59 (dd, J=10.4, 5.1, 1H), 3.23 (dd, J=16.8, 5.1, 1H), 2.92-2.87 (m, 3H), 2.91 (s, 3H), 2.43-2.38 (m, 4H), 2.32 (t, J=6.8, 2H), 2.11-2.04 (m, 2H), 1.88 (bs, 1H).

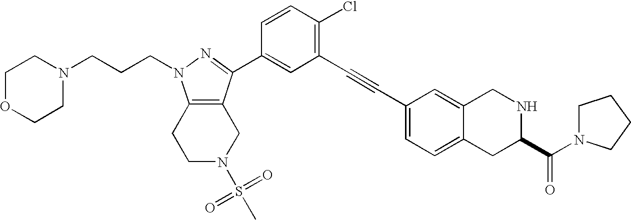

### Example 196

### (3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline

The title compound was prepared using methods analogous to those described for Examples 190-192, substituting 7-hydroxy-(3R)-1,2,3,4-tetrahydroisoquinoline-3-carboxylic acid for 7-hydroxy-(3S)-1,2,3,4-tetrahydroisoquinoline-3-carboxylic acid in Example 190, Step A, and pyrrolidine for methylamine in Example 192. HPLC: Rₜ=4.24. MS (ESI): mass calcd. for C₃₆H₄₃ClN₆O₄S, 690.3; m/z found, 691.3 [M+H]⁺. ¹H NMR (CDCl₃): 7.78 (d, J=1.8, 1H), 7.49 (dd, J=8.4, 2.0, 1H), 7.45 (d, J=8.3, 1H), 7.37 (dd, J=7.9, 1.4, 1H), 7.29 (s, 1H), 7.10 (d, J=7.9, 1H), 4.54 (s, 2H), 4.13-4.08 (m, 4H), 3.77 (dd, J=11.0, 4.1, 1H), 3.72-3.68 (m, 5H), 3.65 (t, J=5.7, 2H), 3.58-3.44 (m, 3H), 3.00 (dd, J=16.6, 11.1, 1H), 2.91 (s, 3H), 2.91-2.87 (m, 2H), 2.83 (dd, J=16.8, 3.1, 1H), 2.43-2.39 (m, 4H), 2.32 (t, J=6.8, 2H), 2.11-2.05 (m, 2H), 2.02-1.87 (m, 6H).

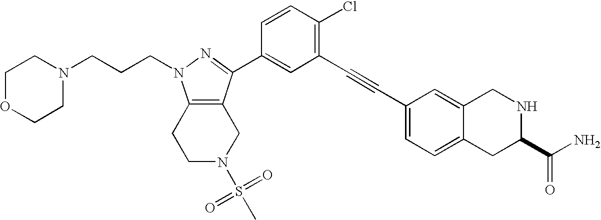

### Example 197

### (3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide

The title compound was prepared using methods analogous to those described for Example 195, substituting 7-hydroxy-(3R)-1,2,3,4-tetrahydroisoquinoline-3-carboxylic acid for 7-hydroxy-(3S)-1,2,3,4-tetrahydroisoquinoline-3-carboxylic acid in Example 190, Step A. HPLC: Rₜ=4.10. MS (ESI): mass calcd. for C₃₂H₃₇ClN₆O₄S, 636.2; m/z found, 637.2 [M+H]⁺. ¹H NMR (CDCl₃): 7.78 (d, J=1.9, 1H), 7.49 (dd, J=8.4, 2.0, 1H), 7.45 (d, J=8.4, 1H), 7.40 (dd, J=7.8, 1.3, 1H), 7.30 (s, 1H), 7.16 (d, J=7.9, 1H), 7.06 (bs, 1H), 5.72 (bs, 1H), 4.53 (s, 2H), 4.10 (t, J=6.8, 2H), 4.03 (s, 2H), 3.70 (t, J=4.6, 4H), 3.65 (t, J=5.8, 2H), 3.59 (dd, J=10.4, 5.2, 1H), 3.23 (dd, J=16.9, 5.1, 1H), 2.93-2.85 (m, 3H), 2.90 (s, 3H), 2.43-2.38 (m, 4H), 2.32 (t, J=6.8, 2H), 2.11-2.04 (m, 2H).

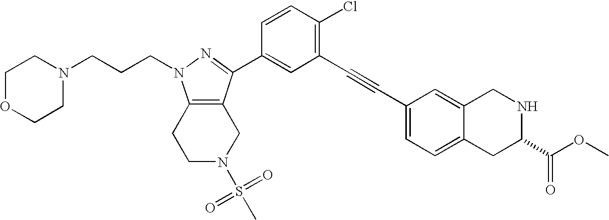

### Example 198

### Methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate

To an ice-cooled solution of 2-(1,1-dimethylethyl) 3-methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate (160 mg, 0.21 mmol) in CH₂Cl₂ was added TFA (0.8 mL). After 3 h, the mixture was diluted with satd. aq NaHCO₃ and extracted with CH₂Cl₂ (3×). The combined organic extracts were dried and concentrated to afford the title compound as a white foam (133 mg, 96%). HPLC: Rₜ=4.22. MS (ESI): mass calcd. for C₃₃H₃₈ClN₅O₅S, 651.1; m/z found, 652.1 [M+H]⁺. ¹H NMR (CDCl₃): 7.77 (d, J=1.9, 1H), 7.48 (dd, J=8.4, 2.0, 1H), 7.44 (d, J=8.4, 1H), 7.37 (dd, J=7.8, 1.5, 1H), 7.29 (s, 1H), 7.12 (d, J=7.9, 1H), 4.52 (s, 2H), 4.13-4.07 (m, 4H), 3.78 (s, 3H), 3.78-3.74 (m, 1H), 3.69 (t, J=4.6, 4H), 3.64 (t, J=5.7, 2H), 3.11 (dd, J=16.6, 4.6, 1H), 3.02-2.94 (m, 1H), 2.90 (s, 3H), 2.90-2.86 (m, 2H), 2.43-2.38 (m, 4H), 2.32 (t, J=6.8, 2H), 2.13 (bs, 1H), 2.10-2.13 (m, 2H).

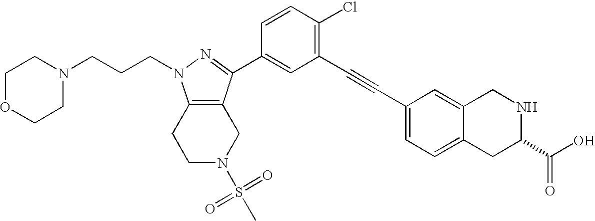

### Example 199

### (3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid

The title compound was prepared from (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-di methylethyl)oxy]carbonyl}-1,2,3,4-tetrahydroisoquinoline-3-carboxylic acid using methods analogous to those described for Example 189. HPLC: Rₜ=4.10. MS (ESI): mass calcd. for C₃₂H₃₆ClN₅O₅S, 637.2; m/z found, 638.1 [M+H]⁺. ¹H NMR: 7.86 (d, J=1.9, 1H), 7.60 (dd, J=8.5, 2.0, 1H), 7.56 (d, J=8.4, 1H), 7.55-7.49 (m, 3H), 7.37 (d, J=7.9, 1H), 4.55-4.47 (m, 1H), 4.51 (s, 2H), 4.45-4.40 (m, 1H), 4.23 (t, J=6.5, 2H), 4.06-4.00 (m, 2H), 3.76-3.68 (m, 2H), 3.65 (t, J=5.8, 2H), 3.56-3.47 (m, 3H), 3.33-3.29 (m, 3H), 3.28-3.22 (m, 2H), 3.17-3.09 (m, 2H), 2.98 (s, 3H), 2.92 (t, J=5.6, 2H), 2.38-2.30 (m, 2H).

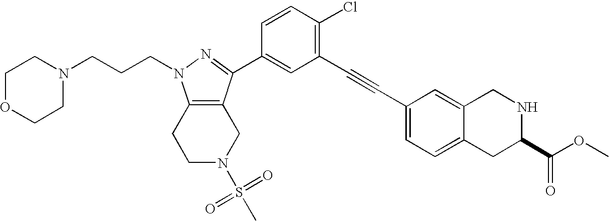

### Example 200

### Methyl (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate

The title compound was prepared from 2-(1,1-dimethylethyl) 3-methyl (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate using methods analogous to those described for Example 198. HPLC: Rₜ=4.23. MS (ESI): mass calcd. for C₃₃H₃₈ClN₅O₅S, 651.2; m/z found, 652.2 [M+H]⁺. ¹H NMR (CDCl₃): 7.77 (d, J=1.9, 1H), 7.48 (dd, J=8.4, 2.0, 1H), 7.44 (d, J=8.4, 1H), 7.37 (dd, J=7.9, 1.4, 1H), 7.29 (s, 1H), 7.12 (d, J=7.9, 1H), 4.52 (s, 2H), 4.14-4.08 (m, 4H), 3.79 (s, 3H), 3.78-3.74 (m, 1H), 3.69 (t, J=4.6, 4H), 3.64 (t, J=5.8, 2H), 3.11 (dd, J=16.6, 4.5, 1H), 2.98 (dd, J=16.6, 9.9, 1H), 2.90 (s, 3H), 2.90-2.87 (m, 2H), 2.42-2.38 (m, 4H), 2.32 (t, J=6.8, 2H), 2.20 (bs, 1H), 2.10-2.03 (m, 2H).

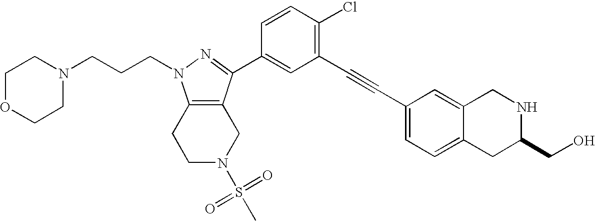

### Example 201

### [(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol

To a cooled (−78° C.) solution of methyl (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate (67 mg, 0.10 mmol) in THF was added DIBAL-H (1.5 M in toluene; 0.35 mL, 0.53 mmol). The reaction mixture was stirred at −78° C. for 1 h and at 0° C. for 30 min. After cooling to −78° C., the reaction was quenched with MeOH (1 mL), diluted with 1 N NaOH, and extracted with CH₂Cl₂ (3×). The combined organic extracts were dried and concentrated to give a brown oil. Purification (SiO₂: 0 to 5% 2 M NH₃ in MeOH/CH₂Cl₂) afforded the title compound as a yellow oil (19 mg, 30%). HPLC: Rₜ=4.13. MS (ESI): mass calcd. for C₃₂H₃₈ClN₅O₄S, 623.2; m/z found, 624.2 [M+H]⁺. ¹H NMR (CDCl₃): 7.77 (d, J=1.9, 1H), 7.48 (dd, J=8.4, 2.0, 1H), 7.44 (d, J=8.4, 1H), 7.39-7.35 (m, 1H), 7.29 (s, 1H), 7.09 (d, J=7.9, 1H), 4.53 (s, 2H), 4.10 (t, J=6.8, 2H), 4.07 (s, 2H), 3.82 (dd, J=10.8, 3.4, 1H), 3.70 (t, J=4.6, 4H), 3.65 (t, J=5.8, 2H), 3.55 (dd, J=10.6, 8.1, 1H), 3.15-3.08 (m, 1H), 2.91 (s, 3H), 2.91-2.87 (m, 2H), 2.75 (dd, J=16.7, 4.0, 1H), 2.61 (dd, J=16.7, 10.8, 1H), 2.43-2.38 (m, 4H), 2.33 (t, J=6.8, 2H), 2.21 (bs, 2H), 2.11-2.04 (m, 2H).

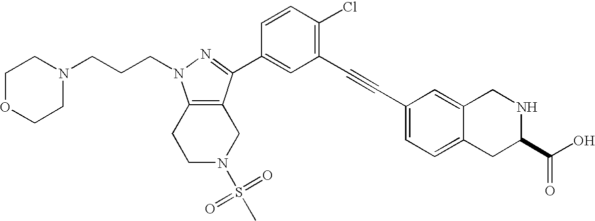

### Example 202

### (3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid

The title compound was prepared from (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-di methylethyl)oxy]carbonyl}-1,2,3,4-tetrahydroisoquinoline-3-carboxylic acid using methods analogous to those described for Example 199. HPLC: Rₜ=4.16. MS (ESI): mass calcd. for C₃₂H₃₆ClN₅O₅S, 637.2; m/z found, 638.2 [M+H]⁺. ¹H NMR: 7.85 (d, 1.8, 1H), 7.59 (dd, J=8.4, 2.0, 1H), 7.55 (d, J=8.4, 1H), 7.53-7.49 (m, 3H), 7.36 (d, J=8.0, 1H), 4.55-4.47 (m, 1H), 4.50 (s, 2H), 4.46-4.40 (m, 1H), 4.22 (t, J=6.5, 2H), 4.05-3.98 (m, 2H), 3.77-3.69 (m, 2H), 3.64 (t, J=5.7, 2H), 3.55-3.47 (m, 3H), 3.33-3.29 (m, 3H), 3.28-3.22 (m, 2H), 3.17-3.09 (m, 2H), 2.98 (s, 3H), 2.91 (t, J=5.6, 2H), 2.37-2.30 (m, 2H).

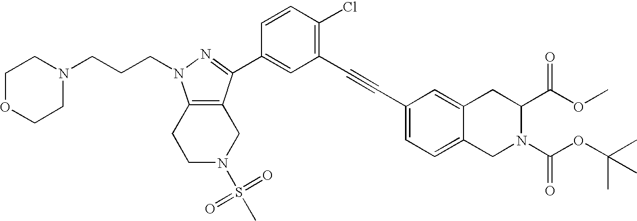

### Example 203

### 2-(1,1-Dimethylethyl) 3-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate

The title compound was prepared from 6-hydroxy-1,2,3,4-tetrahydroisoquinoline-3-carboxylic acid using methods analogous to those described for Example 190. HPLC: Rₜ=5.47. MS (ESI): mass calcd. for C₃₂H₃₈ClN₅O₄S, 751.3; m/z found, 752.1 [M+H]⁺. ¹H NMR (CDCl₃): 7.78 (d, J=1.9, 1H), 7.50 (dd, J=8.4, 2.1, 1H), 7.45 (d, J=8.4, 1H), 7.40 (s, 1H), 7.39-7.36 (m, 1H), 7.17 (d, J=7.9, 0.55H, major rotamer), 7.12 (d, J=7.9, 0.45H, minor rotamer), 5.19 (dd, J=6.1, 2.7, 0.55H, major rotamer), 4.84 (t, J=5.2, 0.45H, minor rotamer), 4.74 (dd, J=17.0, 7.5, 1H), 4.59-4.49 (m, 1H), 4.54 (s, 2H), 4.10 (t, J=6.8, 2H), 3.70 (t, J=4.6, 4H), 3.68-3.64 (m, 2H), 3.65 (s, 1.65H, major rotamer), 3.64 (s, 1.35H, minor rotamer), 3.29 (dd, J=16.0, 2.5, 0.55H, major rotamer), 3.18 (d, J=5.3, 1H), 3.15 (d, J=6.3, 0.45H, minor rotamer), 2.91 (s, 3H), 2.91-2.88 (m, 2H), 2.43-2.39 (m, 4H), 2.33 (t, J=6.8, 2H), 2.11-2.04 (m, 2H), 1.54 (s, 5H, major rotamer), 1.47 (s, 4H, minor rotamer).

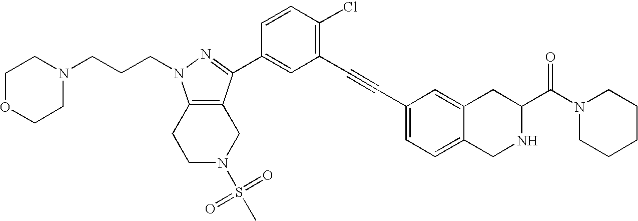

### Example 204

### 6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline

The title compound was prepared from 2-(1,1-dimethylethyl) 3-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate using methods analogous to those described for Examples 191-192. HPLC: Rₜ=5.47. MS (ESI): mass calcd. for C₃₇H₄₅ClN₆O₄S, 704.3; m/z found, 705.2 [M+H]⁺. ¹H NMR (CDCl₃): 7.78 (d, J=1.9, 1H), 7.50 (dd, J=8.4, 2.1, 1H), 7.45 (d, J=8.3, 1H), 7.37 (d, J=7.9, 1H), 7.35 (s, 1H), 7.05 (d, J=7.9, 1H), 4.54 (s, 2H), 4.15-4.12 (m, 2H), 4.10 (t, J=6.9, 2H), 3.95 (dd, J=10.9, 4.2, 1H), 3.70 (t, J=4.6, 4H), 3.66 (t, J=5.8, 2H), 3.64-3.60 (m, 2H), 3.52-3.48 (m, 2H), 2.97-2.92 (m, 1H), 2.91 (s, 3H), 2.91-2.88 (m, 2H), 2.80 (dd, J=16.5, 3.7, 1H), 2.43-2.39 (m, 4H), 2.33 (t, J=6.8, 2H), 2.11-2.04 (m, 2H), 1.83 (bs, 1H), 1.71-1.57 (m, 6H).

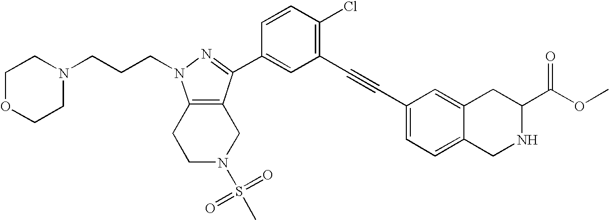

### Example 205

### Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate

The title compound was prepared from 2-(1,1-dimethylethyl) 3-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate using methods analogous to those described for Example 198. HPLC: Rₜ=4.17. MS (ESI): mass calcd. for C₃₃H₃₈ClN₅O₅S, 651.2; m/z found, 652.1 [M+H]⁺. ¹H NMR (CDCl₃): 7.79 (d, J=1.8, 1H), 7.49 (dd, J=8.4, 2.0, 1H), 7.45 (d, J=8.4, 1H), 7.39-7.36 (m, 2H), 7.04 (d, J=8.0, 1H), 4.53 (s, 2H), 4.16-4.08 (m, 4H), 3.79 (s, 3H), 3.78-3.74 (m, 2H), 3.70 (t, J=4.6, 4H), 3.66 (t, J=5.8, 2H), 3.11 (dd, J=16.4, 4.5, 1H), 2.98 (dd, J=16.3, 9.9, 1H), 2.91 (s, 3H), 2.91-2.88 (m, 2H), 2.43-2.38 (m, 4H), 2.33 (t, J=6.8, 2H), 2.11-2.04 (m, 2H).

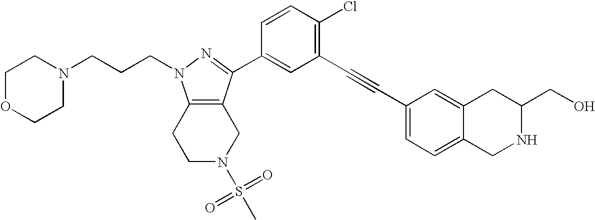

### Example 206

### [6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol

The title compound was prepared from methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate using methods analogous to those described for Example 201. HPLC: Rₜ=4.10. MS (ESI): mass calcd. for C₃₂H₃₈ClN₅O₄S, 623.2; m/z found, 624.2 [M+H]⁺. ¹H NMR (CDCl₃): 7.78 (d, J=1.8, 1H), 7.49 (dd, J=8.4, 2.0, 1H), 7.45 (d, J=8.4, 1H), 7.37 (d, J=8.1, 1H), 7.37 (s, 1H), 7.05 (d, J=7.8, 1H), 5.80 (s, 2H), 4.13-4.07 (m, 4H), 3.82 (dd, J=10.7, 3.5, 1H), 3.70 (t, J=4.6, 4H), 3.66 (t, J=5.8, 2H), 3.57-3.52 (m, 1H), 3.27-3.21 (m, 1H), 3.15-3.08 (m, 1H), 2.91 (s, 3H), 2.91-2.87 (m, 2H), 2.75 (dd, J=16.2, 3.8, 1H), 2.57 (dd, J=16.0, 10.9, 1H), 2.44-2.38 (m, 4H), 2.33 (t, J=6.8, 2H), 2.11-2.04 (m, 2H), 1.68-1.60 (bm, 1H).

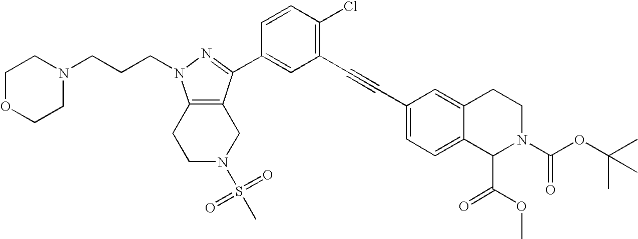

### Example 207

### 2-(1,1-Dimethylethyl) 1-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-1,2(1H)-dicarboxylate

The title compound was prepared from 6-hydroxy-1,2,3,4-tetrahydro-isoquinoline-1-carboxylic acid using methods analogous to those described for Example 190. HPLC: Rₜ=5.73. MS (ESI): mass calcd. for C₃₈H₄₆ClN₅O₇S, 751.3; m/z found, 752.3 [M+H]⁺. ¹H NMR (CDCl₃): 7.79 (d, J=2.0, 1H), 7.50 (dd, J=8.4, 2.0, 1H), 7.50-7.44 (m, 2H), 7.45 (d, J=8.4, 1H), 7.41 (s, 1H), 5.62 (s, 0.4H, minor rotamer), 5.46 (s, 0.6H, major rotamer), 4.54 (s, 2H), 4.13-4.08 (m, 2H), 3.81-3.75 (m, 2H), 3.73 (s, 3H), 3.70 (t, J=4.6, 4H), 3.66 (t, J=5.8, 2H), 2.98-2.93 (m, 2H), 2.91 (s, 3H), 2.91-2.87 (m, 2H), 2.43-2.39 (m, 4H), 2.33 (t, J=6.8, 2H), 2.11-2.04 (m, 2H), 1.51 (s, 3.6H, minor rotamer), 1.49 (5.4H, major rotamer).

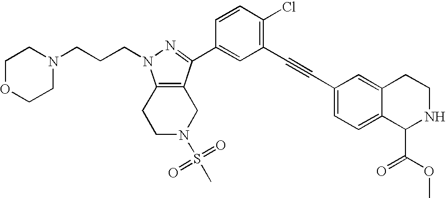

### Example 208

### Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-1-carboxylate

The title compound was prepared from 2-(1,1-dimethylethyl) 1-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenylethynyl)-3,4-dihydroisoquinoline-1,2(1H)-dicarboxylate using methods analogous to those described for Example 198. HPLC: Rₜ=4.23. MS (ESI): mass calcd. for C₃₃H₃₈ClN₅O₅S, 651.2; m/z found, 652.3 [M+H]⁺. ¹H NMR (CDCl₃): 7.78 (d, J=1.9, 1H), 7.49 (dd, J=8.4, 2.0, 1H), 7.45 (d, J=8.4, 1H), 7.40 (dd, J=7.9, 1.6, 1H), 7.38-7.35 (m, 2H), 4.76 (s, 1H), 4.54 (s, 2H), 4.10 (t, J=6.8, 2H), 3.78 (s, 3H), 3.70 (t, J=4.6, 4H), 3.66 (t, J=5.8, 2H), 3.32-3.25 (m, 1H), 3.09 (dt, J=12.6, 5.4, 1H), 2.91 (s, 3H), 2.91-2.88 (m, 2H), 2.87-2.75 (m, 2H), 2.43-2.39 (m, 4H), 2.33 (t, J=6.8, 2H), 2.11-2.04 (m, 1H).

The compounds in Examples 209-219 were prepared using methods analogous to those described for Example 177, substituting the appropriate amines for 4-chlorobenzylamine in Step A and 2-(4-piperidinyl)pyridine in Step B.

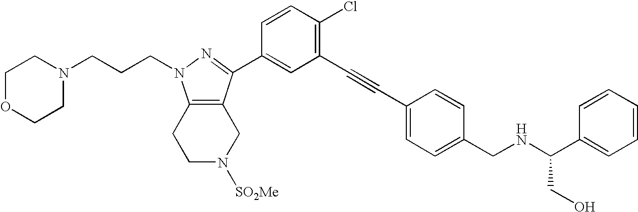

### Example 209

### (2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}amino)-2-phenylethanol

MS (ESI): mass calcd. for C₃₇H₄₂ClN₅O₄S, 687.3; m/z found, 688.3 [M+H]⁺. ¹H NMR (CDCl₃): 7.80 (d, J=1.6, 1H), 7.55 (d, J=8.2, 2H), 7.51-7.43 (m, 2H), 7.41-7.36 (m, 2H), 7.35-7.28 (m, 5H), 4.54 (s, 2H), 4.10 (t, J=6.8, 2H), 3.84-3.73 (m, 3H), 3.72-3.68 (m, 4H), 3.68-3.56 (m, 4H), 2.91 (s, 3H), 2.90-2.87 (m, 2H), 2.46-2.37 (m, 4H), 2.33 (t, J=6.8, 2H), 2.13-2.03 (m, 2H).

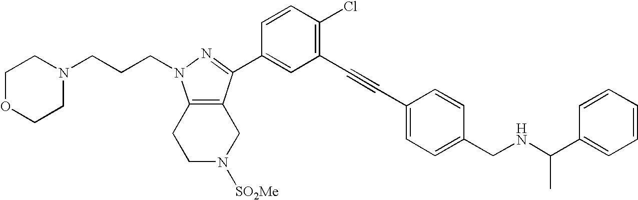

### Example 210

### N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine

MS (ESI): mass calcd. for C₃₇H₄₂ClN₅O₃S, 671.3; m/z found, 672.3 [M+H]⁺. ¹H NMR (CDCl₃): 7.80 (d, J=1.7, 1H), 7.54 (d, J=8.2, 2H), 7.50-7.44 (m, 2H), 7.36 (d, J=4.4, 4H), 7.31-7.25 (m, 3H), 4.54 (s, 2H), 4.10 (t, J=6.8, 2H), 3.80 (q, J=6.6, 1H), 3.73-3.68 (m, 4H), 3.68-3.59 (m, 4H), 2.91 (s, 3H), 2.90-2.87 (m, 2H), 2.45-2.37 (m, 4H), 2.33 (t, J=6.8, 2H), 2.12-2.04 (m, 2H), 1.38 (d, J=6.6, 3H).

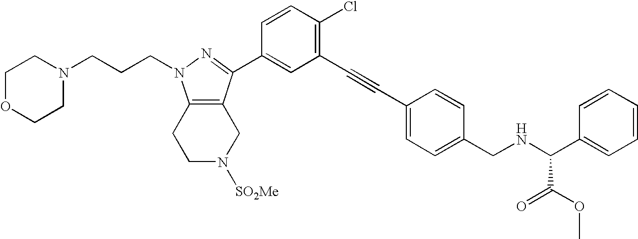

### Example 211

### Methyl (2R)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}amino)(phenyl)ethanoate

MS (ESI): mass calcd. for C₃₈H₄₂ClN₅O₅S, 715.3; m/z found, 716.3 [M+H]⁺. ¹H NMR (CDCl₃): 7.80 (d, J=1.8, 1H), 7.55 (d, J=8.2, 2H), 7.50-7.44 (m, 2H), 7.40-7.35 (m, 4H), 7.35-7.31 (m, 3H), 4.54 (s, 2H), 4.38 (s, 1H), 4.11 (t, J=6.8, 2H), 3.75 (s, 2H), 3.74-3.68 (m, 7H), 3.66 (t, J=5.8, 2H), 2.91 (s, 3H), 2.90-2.87 (m, 2H), 2.48-2.39 (m, 4H), 2.34 (t, J=6.6, 2H), 2.14-2.03 (m, 2H).

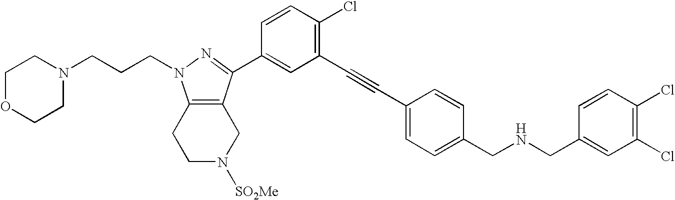

### Example 212

### 1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3,4-dichlorophenyl)methyl]methanamine

MS (ESI): mass calcd. for C₃₇H₄₀ClF₃N₆O₂S, 724.3; m/z found, 725.2 [M+H]⁺. ¹H NMR (CDCl₃): 7.80 (d, J=1.8, 1H), 7.57 (d, J=8.2, 2H), 7.51-7.44 (m, 3H), 7.40 (d, J=8.2, 1H), 7.35 (d, J=8.2, 2H), 7.19 (dd, J=8.2, 1.9, 1H), 4.54 (s, 2H), 4.11 (t, J=6.8, 2H), 3.83-3.76 (m, 4H), 3.73-3.69 (m, 4H), 3.66 (t, J=5.7, 2H), 2.91 (s, 3H), 2.90-2.87 (m, 2H), 2.49-2.38 (m, 4H), 2.34 (t, J=6.8, 2H), 2.13-2.04 (m, 2H).

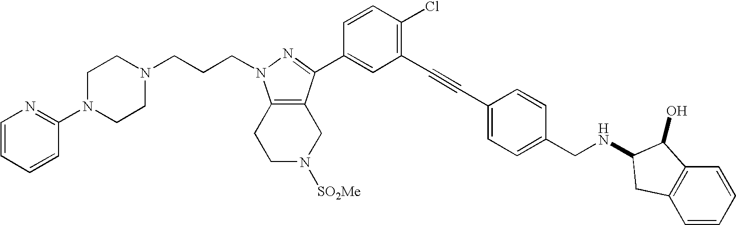

### Example 213

### (1S,2R)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)amino]-2,3-dihydro-1H-inden-1-ol

MS (ESI): mass calcd. for C₄₃H₄₆ClN₇O₃S, 775.3; m/z found, 776.2 [M+H]⁺. ¹H NMR (CDCl₃): 8.20-8.18 (m, 1H), 7.81 (d, J=1.9, 1H), 7.63-7.61 (m, 1H), 7.60-7.59 (m, 1H), 7.53-7.45 (m, 3H), 7.42 (d, J=8.2, 2H), 7.26-7.21 (m, 4H), 6.68-6.60 (m, 2H), 4.55 (s, 2H), 4.44-4.39 (m, 1H), 4.18-4.10 (m, 3H), 4.04 (d, J=2.4, 2H), 3.66 (t, J=5.8, 2H), 3.58-3.49 (m, 4H), 3.11-2.94 (m, 2H), 2.94-2.91 (m, 2H), 2.90 (s, 3H), 2.59-2.49 (m, 4H), 2.44-2.32 (m, 2H), 2.20-2.09 (m, 2H).

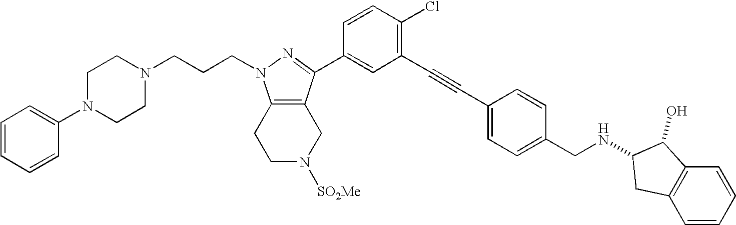

### Example 214

### (1R,2S)-1-[({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)amino]-2,3-dihydro-1H-inden-2-ol

MS (ESI): mass calcd. for C₄₄H₄₇ClN₆O₃S, 774.3; m/z found, 775.2 [M+H]⁺. ¹H NMR (CDCl₃): 7.81 (d, J=1.9, 1H), 7.60 (d, J=8.2, 2H), 7.53-7.44 (m, 2H), 7.42 (d, J=8.2, 2H), 7.30-7.21 (m, 6H), 6.96-6.90 (m, 2H), 6.86 (t, J=7.3, 1H), 4.54 (s, 2H), 4.44-4.39 (m, 1H), 4.16-4.10 (m, 3H), 4.04 (d, J=2.5, 2H), 3.65 (t, J=5.8, 2H), 3.22-3.16 (m, 4H), 3.10-2.94 (m, 2H), 2.93-2.90 (m, 2H), 2.90 (s, 3H), 2.63-2.54 (m, 4H), 2.38 (t, J=6.8, 2H), 2.20-2.07 (m, 2H).

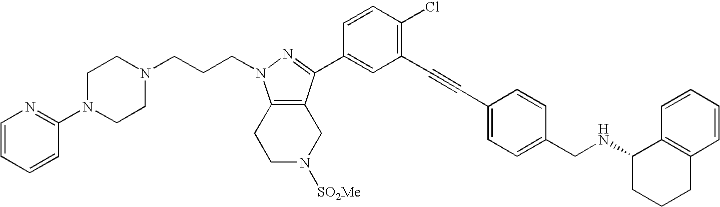

### Example 215

### (1R)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine

MS (ESI): mass calcd. for C₄₄H₄₈ClN₇O₂S, 773.3; m/z found, 774.2 [M+H]⁺. ¹H NMR (CDCl₃). 8.19 (dd, J=4.9, 1.3, 1H), 7.81 (d, J=1.9, 1H), 7.57 (d, J=8.2, 2H), 7.52-7.44 (m, 3H), 7.42 (d, J=8.2, 2H), 7.38-7.35 (m, 1H), 7.18-7.12 (m, 2H), 7.11-7.06 (m, 1H), 6.68-6.59 (m, 2H), 4.55 (s, 2H), 4.13 (t, J=6.8, 2H), 3.99-3.86 (m, 2H), 3.81 (t, J=4.9, 1H), 3.66 (t, J=5.7, 2H), 3.56-3.50 (m, 4H), 2.94-2.91 (m, 2H), 2.90 (s, 3H), 2.88-2.69 (m, 2H), 2.55-2.50 (m, 4H), 2.37 (t, J=6.7, 2H), 2.17-2.07 (m, 2H), 2.06-1.97 (m, 1H), 1.94-1.88 (m, 2H), 1.80-1.70 (m, 1H).

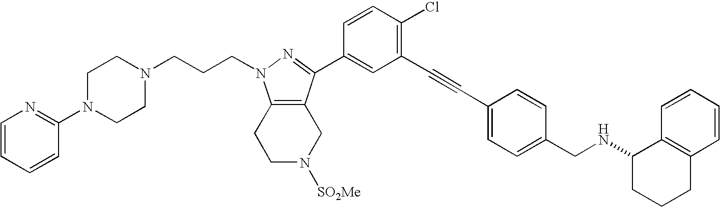

### Example 216

### (1S)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine

MS (ESI): mass calcd. for C₄₄H₄₈ClN₇O₂S, 773.3; m/z found, 774.2 [M+H]⁺. ¹H NMR (CDCl₃): 8.19 (dd, J=4.9, 1.2, 1H), 7.81 (d, J=1.8, 1H), 7.57 (d, J=8.2, 2H), 7.52-7.44 (m, 3H), 7.42 (d, J=8.3, 2H), 7.39-7.34 (m, 1H), 7.18-7.13 (m, 2H), 7.11-7.07 (m, 1H), 6.68-6.59 (m, 2H), 4.55 (s, 2H), 4.13 (t, J=6.8, 2H), 3.99-3.86 (m, 2H), 3.81 (t, J=4.9, 1H), 3.66 (t, J=5.8, 2H), 3.56-3.50 (m, 4H), 2.94-2.91 (m, 2H), 2.90 (s, 3H), 2.89-2.68 (m, 2H), 2.55-2.51 (m, 4H), 2.37 (t, J=6.8, 2H), 2.17-2.08 (m, 2H), 2.06-1.96 (m, 1H), 1.94-1.87 (m, 2H), 1.80-1.71 (m, 1H).

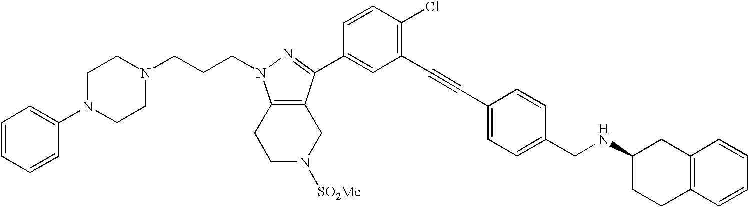

### Example 217

### (1R)—N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine

MS (ESI): mass calcd. for C₄₅H₄₉ClN₆O₂S, 772.3; m/z found, 773.3 [M+H]⁺. ¹H NMR (CDCl₃): 7.81 (d, J=1.9, 1H), 7.57 (d, J=8.2, 2H), 7.52-7.44 (m, 2H), 7.42 (d, J=8.2, 2H), 7.39-7.34 (m, 1H), 7.29-7.23 (m, 2H), 7.19-7.12 (m, 2H), 7.11-7.07 (m, 1H), 6.93 (d, J=7.9, 2H), 6.86 (t, J=7.3, 1H), 4.54 (s, 2H), 4.13 (t, J=6.8, 2H), 3.99-3.86 (m, 2H), 3.81 (t, J=4.9, 1H), 3.66 (t, J=5.7, 2H), 3.23-3.14 (m, 4H), 2.94-2.90 (m, 2H), 2.90 (s, 3H), 2.87-2.69 (m, 2H), 2.60-2.56 (m, 4H), 2.38 (t, J=6.8, 2H), 2.17-2.07 (m, 2H), 2.07-1.95 (m, 1H), 1.95-1.87 (m, 2H), 1.80-1.69 (m, 1H).

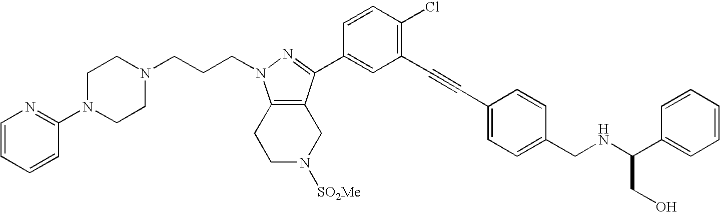

### Example 218

### (2S)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)amino]-2-phenylethanol

MS (ESI): mass calcd. for C₄₂H₄₆ClN₇O₃S, 763.3; m/z found, 764.2 [M+H]⁺. ¹H NMR (CDCl₃): 8.21-8.16 (m, 1H), 7.81 (d, J=1.8, 1H), 7.55 (d, J=8.2, 2H), 7.52-7.44 (m, 3H), 7.41-7.36 (m, 2H), 7.35-7.28 (m, 5H), 6.68-6.60 (m, 2H), 4.54 (s, 2H), 4.13 (t, J=6.8, 2H), 3.86-3.69 (m, 3H), 3.68-3.56 (m, 4H), 3.55-3.51 (m, 4H), 2.94-2.91 (m, 2H), 2.90 (s, 3H), 2.56-2.51 (m, 4H), 2.38 (t, J=6.8, 2H), 2.17-2.08 (m, 2H).

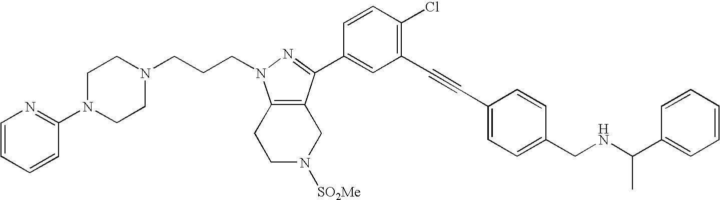

### Example 219

### N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1-phenylethanamine

MS (ESI): mass calcd. for C₄₂H₄₆ClN₇O₂S, 747.3; m/z found, 748.2 [M+H]⁺. ¹H NMR (CDCl₃): 8.20-8.17 (m, 1H), 7.81 (d, J=1.8, 1H), 7.55 (d, J=8.2, 2H), 7.51-7.44 (m, 3H), 7.36 (d, J=4.4, 4H), 7.29 (d, J=8.2, 2H), 7.28-7.24 (m, 1H), 6.68-6.59 (m, 2H), 4.54 (s, 2H), 4.13 (t, J=6.8, 2H), 3.81 (q, J=6.6, 1H), 3.69-3.62 (m, 4H), 3.56-3.50 (m, 4H), 2.94-2.90 (m, 2H), 2.90 (s, 3H), 2.55-2.50 (m, 4H), 2.37 (t, J=6.8, 2H), 2.17-2.07 (m, 2H), 1.38 (d, J=6.6, 3H).

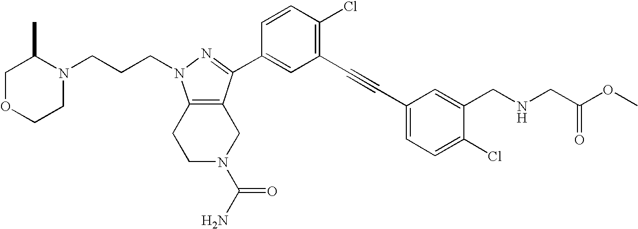

### Example 220

### Methyl N-{[5-({5-[5-(aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl}ethynyl)-2-chlorophenyl]methyl}glycinate

The title compound was prepared using methods analogous to those described for Example 285. MS (ESI): mass calcd. for C₃₃H₃₈Cl₂N₆O₄, 652.2; m/z found, 653.2 [M+H]⁺. ¹H NMR (CDCl₃; mixture of rotamers): 7.78 (d, J=2.1, 1H), 7.65 (d, J=1.9, 1H), 7.56 (d, J=2.1, 0.4H), 7.54 (d, J=2.2, 0.6H), 7.46 (s, 0.6H), 7.45-7.43 (m, 0.8H), 7.42 (d, J=2.0, 0.6H), 7.37 (s, 0.6H), 7.35 (s, 0.4H), 4.69 (s, 2H), 4.59 (s, 2H), 4.16-3.99 (m, 2H), 3.92 (s, 2H), 3.84-3.75 (m, 3H), 3.74 (s, 3H), 3.69-3.59 (m, 2H), 3.48 (s, 2H), 3.24 (dd, J=11.1, 8.8, 1H), 2.88-2.69 (m, 4H), 2.47-2.35 (m, 1H), 2.30-2.20 (m, 2H), 2.13-1.96 (m, 3H), 0.91 (d, J=6.3, 3H).

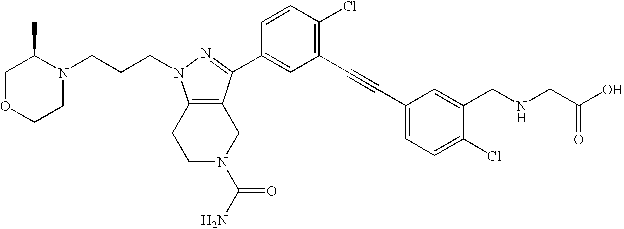

### Example 221

### N-{[5-({5-[5-(Aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl}ethynyl)-2-chlorophenyl]methyl}glycine

A solution of methyl N-{[5-({5-[5-(aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl}ethynyl)-2-chlorophenyl]methyl}glycinate (387 mg, 0.592 mmol) and LiOH (71 mg, 2.96 mmol) in 3:1:1 THF:MeOH:H₂O (3.2 mL) was stirred at rt for 18 h. The mixture was diluted with 1 M HCl and washed with CH₂Cl₂. The aqueous layer was concentrated and purified by reverse-phase HPLC (0-100% MeCN/5 mM NH₄OH in H₂O) to afford the desired product as a yellow solid (226 mg, 60%). MS (ESI): mass calcd. for C₃₂H₃₆Cl₂N₆O₄, 638.2; m/z found, 639.1 [M+H]⁺. ¹H NMR (CDCl₃): 7.78 (s, 1H), 7.73-7.70 (m, 1H), 7.64-7.59 (m, 1H), 7.51-7.38 (m, 3H), 4.59 (s, 2H), 4.26 (s, 2H), 4.12-3.97 (m, 2H), 3.85-3.59 (m, 5H), 3.51 (s, 2H), 3.37 (td, J=3.3, 1.6, 1H), 3.30-3.22 (m, 1H), 2.84-2.70 (m, 4H), 2.51-2.40 (m, 1H), 2.36-2.25 (m, 2H), 2.15-1.92 (m, 2H), 0.94 (d, J=6.3, 3H).

The compounds in Examples 222-224 were prepared using methods analogous to those described for Example 285.

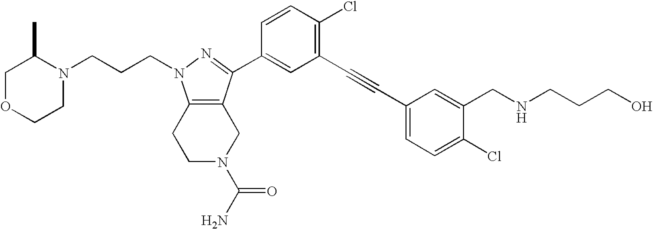

### Example 222

### 3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridine-5-carboxylic acid amide

MS (ESI): mass calcd. for C₃₃H₄₀Cl₂N₆O₃, 638.3; m/z found, 639.2 [M+H]⁺. ¹H NMR (CDCl₃): 7.77 (d, J=2.1, 1H), 7.59-7.54 (m, 2H), 7.47-7.41 (m, 2H), 7.36 (d, J=8.2, 1H), 4.78 (s, 2H), 4.60 (s, 2H), 4.15-3.98 (m, 2H), 3.90 (s, 2H), 3.85-3.81 (m, 2H), 3.81-3.71 (m, 3H), 3.69-3.58 (m, 2H), 3.24 (dd, J=11.1, 8.8, 1H), 2.92-2.88 (m, 2H), 2.86-2.69 (m, 5H), 2.47-2.34 (m, 1H), 2.30-2.19 (m, 2H), 2.12-1.96 (m, 2H), 1.80-1.72 (m, 2H), 0.90 (t, J=7.5, 3H).

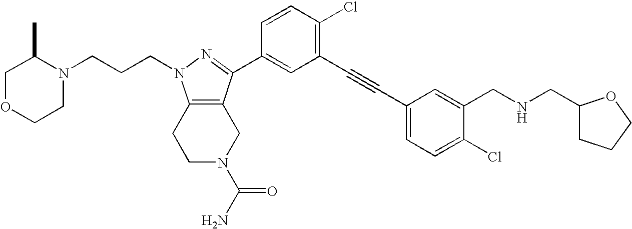

### Example 223

### 3-{4-Chloro-3-[(4-chloro-3-{[(tetrahydrofuran-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide

MS (ESI): mass calcd. for C₃₅H₄₂Cl₂N₆O₃, 664.3; m/z found, 665.2 [M+H]⁺. ¹H NMR (CDCl₃; mixture of rotamers): 7.78 (d, J=2.1, 1H), 7.66 (d, J=1.8, 1H), 7.56 (d, J=2.2, 0.4H), 7.54 (d, J=2.2, 0.6H), 7.47 (s, 0.6H), 7.45 (s, 0.4H), 7.42 (d, J=2.0, 0.4H), 7.40 (d, J=2.0, 0.6H), 7.36 (s, 0.6H), 7.34 (s, 0.4H), 4.67 (d, J=9.8, 3H), 4.17-4.01 (m, 3H), 3.95-3.91 (m, 2H), 3.90-3.82 (m, 1H), 3.81-3.72 (m, 3H), 3.69-3.58 (m, 2H), 3.28-3.19 (m, 1H), 2.85-2.65 (m, 6H), 2.44-2.33 (m, 1H), 2.31-2.20 (m, 2H), 2.12-1.95 (m, 3H), 1.93-1.85 (m, 2H), 1.64-1.53 (m, 1H), 0.91 (d, J=6.3, 3H).

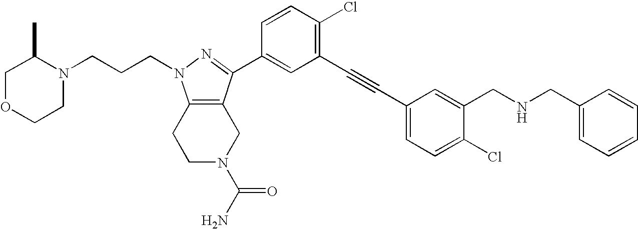

### Example 224

### 3-{4-Chloro-3-[(4-chloro-3-{[(phenylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide

MS (ESI): mass calcd. for C₃₇H₄₀Cl₂N₆O₂, 670.3; m/z found, 671.2 [M+H]⁺. ¹H NMR (CDCl₃; mixture of rotamers): 7.79 (d, J=2.1, 1H), 7.66 (d, J=1.9, 1H), 7.55 (d, J=2.2, 0.4H), 7.53 (d, J=2.1, 0.6H), 7.47 (s, 0.6H), 7.45 (s, 0.4H), 7.43 (d, J=2.0, 0.4H), 7.41 (d, J=2.0, 0.6H), 7.40-7.32 (m, 5H), 7.30-7.24 (m, 1H), 4.65-4.59 (m, 3H), 4.15-3.99 (m, 2H), 3.92 (s, 2H), 3.84 (s, 2H), 3.82-3.71 (m, 3H), 3.69-3.58 (m, 2H), 3.24 (dd, J=11.1, 8.8, 1H), 2.89-2.67 (m, 4H), 2.48-2.34 (m, 1H), 2.31-2.19 (m, 2H), 2.12-1.96 (m, 2H), 0.91 (d, J=6.3, 3H).

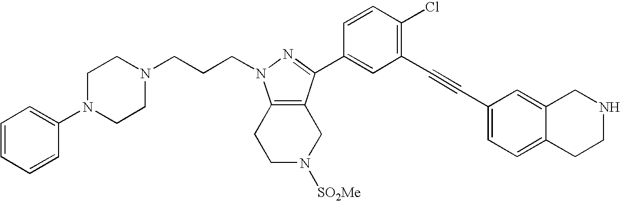

### Example 225

### 7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-1,2,3,4-tetrahydroisoquinoline

The title compound was prepared using methods analogous to those described for Example 135. MS (ESI): mass calcd. for C₃₇H₄₁ClN₆O₂S, 668.3; m/z found, 669.3 [M+H]⁺. ¹H NMR: 7.81-7.79 (m, 1H), 7.52-7.50 (m, 2H), 7.40-7.37 (m, 1H), 7.30 (s, 1H), 7.25 (dd, J=8.7, 7.4, 2H), 7.15 (d, J=7.9, 1H), 6.95 (d, J=7.9, 2H), 6.86 (t, J=7.3, 1H), 4.53 (s, 2H), 4.16 (t, J=6.7, 2H), 4.04 (s, 2H), 3.67 (t, J=5.8, 2H), 3.21-3.15 (m, 6H), 2.96 (s, 3H), 2.95-2.89 (m, 4H), 2.65-2.61 (m, 4H), 2.43 (t, J=7.2, 2H), 2.16-2.11 (m, 2H).

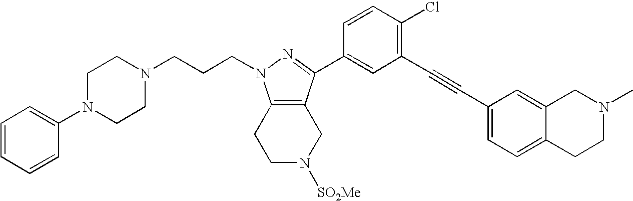

### Example 226

### 7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-methyl-1,2,3,4-tetrahydroisoquinoline

Formaldehyde (37% in H₂O; 0.06 mL) and sodium triacetoxyborohydride (16 mg, 0.075 mmol) were added to a solution of 7-[(2-chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-1,2,3,4-tetrahydroisoquinoline (25 mg, 0.037 mmol) in dichloroethane (0.1 mL) and the resulting mixture was stirred at rt for 18 h. Additional formaldehyde (37% in H₂O, 0.06 mL) and sodium triacetoxyborohydride (16 mg, 0.075 mmol) were added and the mixture was stirred an additional 4 h. The mixture was diluted with 1 M NaOH and extracted with 20% isopropanol in CHCl₃. The organic layer was washed with H₂O, dried, and concentrated. Purification by preparative thin layer chromatography (SiO₂; 3% 2 M NH₃ in MeOH/CH₂Cl₂) afforded the desired product as a white solid (10 mg, 40%). MS (ESI): mass calcd. for C₃₈H₄₃ClN₆O₂S, 682.3; m/z found, 683.3 [M+H]⁺. ¹H NMR (CDCl₃): 7.78 (s, 1H), 7.51-7.48 (m, 2H), 7.42-7.38 (m, 1H), 7.33-7.24 (m, 3H), 7.16 (d, J=7.9, 1H), 6.95 (d, J=8.2, 2H), 6.89 (t, J=7.3, 1H), 4.54 (s, 2H), 4.14 (t, J=6.8, 2H), 3.74-3.70 (m, 2H), 3.67 (t, J=5.7, 2H), 3.26-3.18 (m, 4H), 3.01 (t, J=5.9, 2H), 2.97-2.91 (m, 5H), 2.85 (t, J=5.9, 2H), 2.68-2.62 (m, 4H), 2.55-2.52 (m, 3H), 2.45 (t, J=5.9, 2H), 2.21-2.08 (m, 2H).

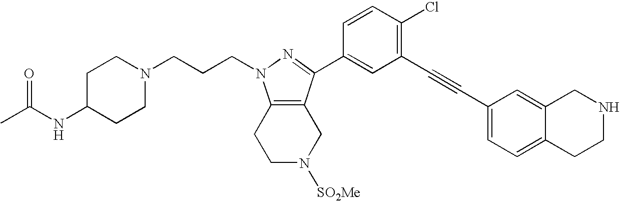

### Example 227

### N-[1-(3-{3-[4-Chloro-3-(1,2,3,4-tetrahydroisoquinolin-7-ylethynyl)phenyl]-5-(methylsulfonyl)-4,5,6,7-tetrahydro-1H-pyrazolo[4,3-c]pyridin-1-yl}propyl)piperidin-4-yl]acetamide

The title compound was prepared using methods analogous to those described for Example 135. MS (ESI): mass calcd. for C₃₄H₄₁ClN₆O₃S, 648.3; m/z found, 649.3 [M+H]⁺. ¹H NMR: 7.83 (s, 1H), 7.55 (s, 2H), 7.37-7.33 (m, 1H), 7.29 (s, 1H), 7.16 (d, J=8.0, 1H), 4.51 (s, 2H), 4.14 (t, J=6.5, 2H), 4.00 (s, 2H), 3.65 (t, J=5.8, 2H), 3.62-3.56 (m, 1H), 3.13 (t, J=6.0, 2H), 2.97 (s, 3H), 2.96-2.92 (m, 2H), 2.91-2.83 (m, 4H), 2.36 (t, J=7.2, 2H), 2.13-1.99 (m, 4H), 1.90 (s, 3H), 1.86-1.78 (m, 2H), 1.53-1.40 (m, 2H).

The compounds in Examples 227-228 were prepared using methods analogous to those described for Example 2.

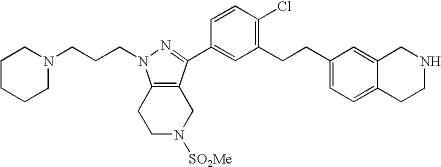

### Example 228

### 7-(2-{2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethyl)-1,2,3,4-tetrahydroisoquinoline

MS (ESI): mass calcd. for C₃₂H₄₂ClN₅O₂S, 595.3; m/z found, 596.2 [M+H]⁺. ¹H NMR: 7.44-7.39 (m, 3H), 7.07-7.04 (m, 2H), 6.92 (s, 1H), 4.40 (s, 2H), 4.12 (m, 2H), 4.05 (s, 2H), 3.64 (t, J=5.8, 2H), 3.21 (t, J=6.1, 2H), 3.10-3.02 (m, 2H), 2.97-2.84 (m, 9H), 2.55-2.42 (m, 4H), 2.41-2.36 (m, 2H), 2.12-2.01 (m, 2H), 1.66-1.56 (m, 4H), 1.53-1.42 (m, 2H).

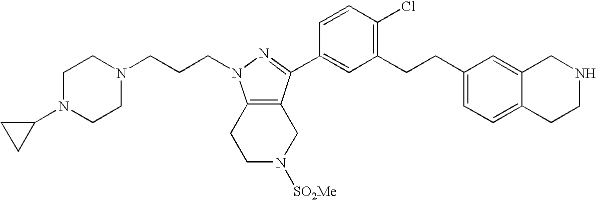

### Example 229

### 7-[2-(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethyl]-1,2,3,4-tetrahydroisoquinoline

MS (ESI): mass calcd. for C₃₄H₄₅ClN₆O₂S, 636.3; m/z found, 637.2 [M+H]⁺. ¹H NMR (CDCl₃): 7.58-7.49 (m, 1H), 7.42-7.37 (m, 1H), 7.34-7.29 (m, 1H), 7.09-7.00 (m, 2H), 6.97-6.86 (m, 1H), 4.48 (s, 2H), 4.09 (t, J=6.8, 2H), 4.00 (s, 2H), 3.66 (t, J=5.8, 2H), 3.13 (t, J=5.9, 2H), 3.06-3.00 (m, 2H), 2.93-2.84 (m, 8H), 2.77 (t, J=5.7, 2H), 2.70-2.55 (m, 4H), 2.47-2.35 (m, 3H), 2.30 (t, J=6.8, 3H), 2.12-1.99 (m, 2H), 0.53-0.35 (m, 4H).

The compounds in Examples 230-237 were prepared according to the methods described for Example 249.

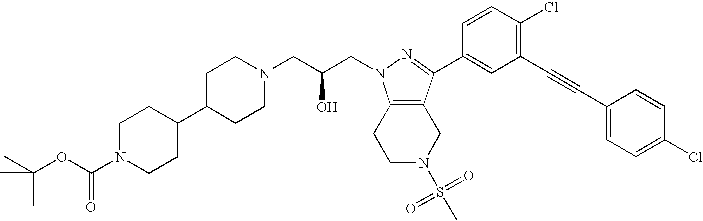

### Example 230

### 1,1-Dimethylethyl 1′-{(2S)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate

MS (ESI): mass calcd. for C₃₉H₄₉Cl₂N₅O₅S, 769.3; m/z found, 770.2 [M+H]⁺. ¹H NMR (CDCl₃): 7.75 (d, J=1.8, 1H), 7.52-7.42 (m, 4H), 7.36 (d, J=7.4, 2H), 4.54 (dd, J=14.5, 6.4, 2H), 4.20-3.95 (m, 5H), 3.70-3.57 (m, 2H), 3.10-3.00 (m, 2H), 2.95-2.85 (m, 2H), 2.85 (s, 3H), 2.70-2.35 (m, 4H), 2.30 (t, J=10.8, 2H), 2.03 (t, J=11.5, 2H), 1.70-1.60 (m, 4H), 1.42 (s, 9H), 1.40-1.10 (m, 4H).

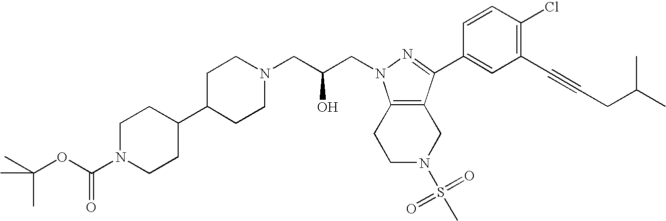

### Example 231

### 1,1-Dimethylethyl 1′-[(2S)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl}-2-hydroxypropyl]-4,4′-bipiperidine-1-carboxylate

MS (ESI): mass calcd. for C₃₇H₅₄ClN₅O₅S, 715.3; m/z found, 716.3 [M+H]⁺. ¹H NMR (CDCl₃): 7.60 (d, J=1.7, 1H), 7.35-7.30 (m, 2H), 4.48 (q, J=14.4, 2H), 4.10-3.85 (m, 5H), 3.65-3.52 (m, 2H), 3.00-2.82 (m, 4H), 2.80 (s, 3H), 2.60-2.50 (m, 2H), 2.35-2.30 (m, 2H), 2.30 (d, J=6.5, 2H), 2.22-2.18 (m, 2H), 1.95-1.85 (m, 2H), 1.65-1.55 (m, 4H), 1.38 (s, 9H), 1.40-1.10 (m, 5H), 1.00 (d, J=6.7, 6H).

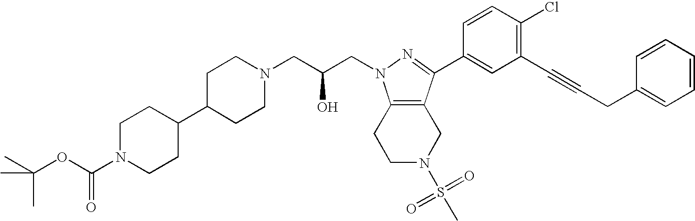

### Example 232

### 1,1-Dimethylethyl 1′-[(2S)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl}-2-hydroxypropyl]-4,4′-bipiperidine-1-carboxylate

MS (ESI): mass calcd. for C₄₀H₅₂ClN₅O₅S, 749.3; m/z found, 750.2 [M+H]⁺. ¹H NMR (CDCl₃): 7.62 (s, 1H), 7.40-7.30 (m, 4H), 7.28 (t, J=7.5, 2H), 7.21-7.17 (m, 1H), 4.48 (q, J=14.4, 2H), 4.10-3.88 (m, 5H), 3.85 (s, 2H), 3.65-3.52 (m, 2H), 3.00-2.92 (m, 2H), 2.85-2.80 (m, 2H), 2.80 (s, 3H), 2.57-2.50 (m, 2H), 2.43-2.33 (m, 2H), 2.23-2.18 (m, 2H), 1.95-1.90 (m, 2H), 1.65-1.55 (m, 4H), 1.38 (s, 9H), 1.40-1.10 (m, 4H).

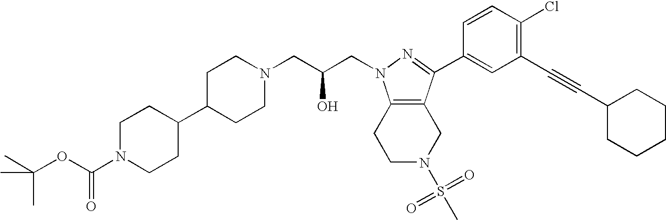

### Example 233

### 1,1-Dimethylethyl 1′-[(2S)-3-{3-[4-chloro-3-(cyclohexylethynyl)phenyl]-5-(methylsulfonyl)-4,5,6,7-tetrahydro-1H-pyrazolo[4,3-c]pyridin-1-yl}-2-hydroxypropyl]-4,4′-bipiperidine-1-carboxylate

MS (ESI): mass calcd. for C₃₉H₅₆ClN₅O₅S, 741.4; m/z found, 742.3 [M+H]⁺. ¹H NMR (CDCl₃): 7.58 (s, 1H), 7.32 (s, 2H), 4.45 (q, J=14.4, 2H), 4.15-3.88 (m, 5H), 3.62-3.52 (m, 2H), 3.07 (q, J=7.3, 1H), 3.00-2.82 (m, 4H), 2.80 (s, 3H), 2.60-2.38 (m, 4H), 2.25 (t, J=11.4, 1H), 1.94 (t, J=11.4, 1H), 1.85-1.80 (m, 2H), 1.75-1.67 (m, 2H), 1.65-1.40 (m, 7H), 1.38 (s, 9H), 1.40-1.10 (m, 9H).

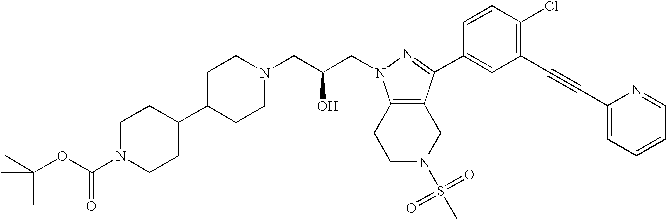

### Example 234

### 1,1-Dimethylethyl 1′-[(2S)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl}-2-hydroxypropyl]-4,4′-bipiperidine-1-carboxylate

MS (ESI): mass calcd. for C₃₈H₄₉ClN₆O₅S, 736.3; m/z found, 737.2 [M+H]⁺. ¹H NMR (CDCl₃): 8.57 (d, J=4.2, 1H), 7.80 (d, J=2.1, 1H), 7.65 (dt, J=7.7, 1.8, 1H), 7.55 (dd, J=7.8, 1.0, 1H), 7.47 (dd, J=8.4, 2.1, 1H), 7.40 (d, J=8.4, 1H), 7.20 (m, 1H), 4.47 (q, J=14.4, 2H), 4.10-3.85 (m, 5H), 3.65-3.50 (m, 2H), 3.00-2.75 (m, 4H), 2.80 (s, 3H), 2.60-2.50 (m, 2H), 2.35-2.25 (m, 2H), 2.17 (t, J=11.5, 2H), 1.90-1.80 (m, 2H), 1.70-1.60 (m, 4H), 1.38 (s, 9H), 1.40-1.10 (m, 4H).

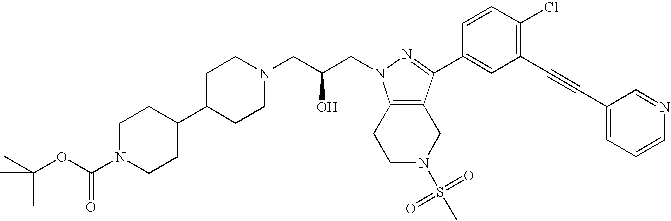

### Example 235

### 1,1-Dimethylethyl 1′-[(2S)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl}-2-hydroxypropyl]-4,4′-bipiperidine-1-carboxylate

MS (ESI): mass calcd. for C₃₈H₄₉ClN₆O₅S, 736.3; m/z found, 737.2 [M+H]⁺. ¹H NMR (CDCl₃): 8.75 (s, 1H), 8.50 (d, J=4.3, 1H), 7.80 (d, J=8.7, 1H), 7.74 (s, 1H), 7.45-7.35 (m, 2H), 7.25-7.22 (m, 1H), 4.48 (q, J=14.4, 2H), 4.10-3.90 (m, 5H), 3.65-3.52 (m, 2H), 3.00-2.93 (m, 2H), 2.87-2.82 (m, 2H), 2.80 (s, 3H), 2.60-2.50 (m, 2H), 2.43-2.33 (m, 2H), 2.22-2.18 (m, 2H), 1.97-1.90 (m, 2H), 1.65-1.55 (m, 4H), 1.38 (s, 9H), 1.40-1.10 (m, 4H).

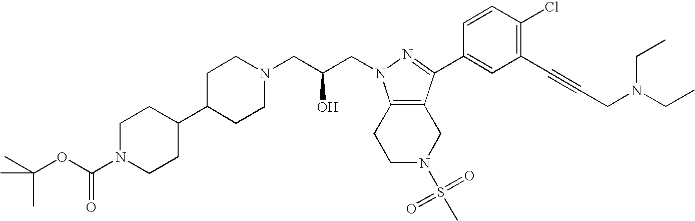

### Example 236

### 1,1-Dimethylethyl 1′-{(2S)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate

MS (ESI): mass calcd. for C₃₈H₅₇ClN₆O₅S, 744.4; m/z found, 745.3 [M+H]⁺. ¹H NMR (CDCl₃): 7.62 (d, J=1.9, 1H), 7.40-7.30 (m, 2H), 4.48 (q, J=14.4, 2H), 4.10-3.88 (m, 5H), 3.65 (s, 2H), 3.65-3.52 (m, 2H), 3.00-2.80 (m, 4H), 2.80 (s, 3H), 2.62 (q, J=7.2, 4H), 2.57-2.50 (m, 2H), 2.35-2.25 (m, 2H), 2.20-2.10 (m, 2H), 1.90-1.80 (m, 2H), 1.65-1.55 (m, 4H), 1.38 (s, 9H), 1.40-1.10 (m, 4H), 1.13 (t, J=7.2, 6H).

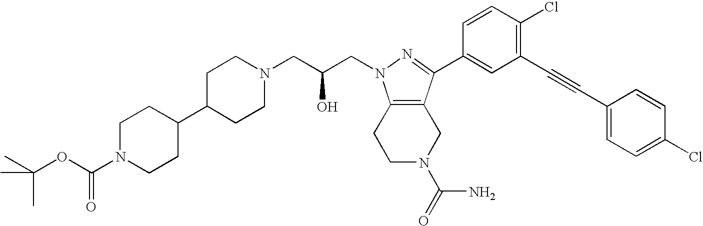

### Example 237

### 1,1-Dimethylethyl 1′-{(2S)-3-[5-(aminocarbonyl)-3-{4-chloro-3-[(4-chlorophenyl)ethynyl]phenyl}-4,5,6,7-tetrahydro-1H-pyrazolo[4,3-c]pyridin-1-yl]-2-hydroxypropyl}-4,4′-bipiperidine-1-carboxylate

MS (ESI): mass calcd. for C₃₉H₄₈Cl₂N₆O₄, 734.3; m/z found, 735.2 [M+H]⁺. ¹H NMR (CDCl₃): 7.80 (d, J=2.0, 1H), 7.55-7.40 (m, 4H), 7.36 (d, J=8.6, 2H), 5.05 (br s, 2H), 4.54 (q, J=14.5, 2H), 4.20-3.95 (m, 5H), 3.80-3.60 (m, 2H), 2.95-2.65 (m, 6H), 2.37 (d, J=6.6, 2H), 2.22-2.15 (m, 2H), 1.95-1.85 (m, 2H), 1.70-1.60 (m, 4H), 1.42 (s, 9H), 1.40-1.10 (m, 4H).

The compounds in Examples 238-239 were prepared according to the methods described for Example 132.

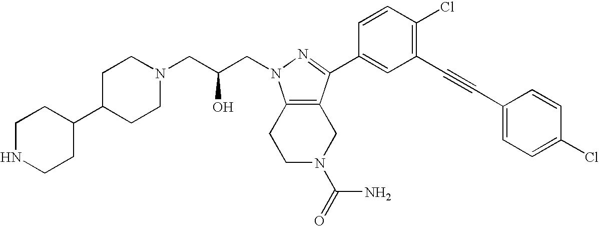

### Example 238

### 1-[(2S)-3-(4,4′-Bipiperidin-1-yl)-2-hydroxypropyl]-3-{4-chloro-3-[(4-chlorophenyl)ethynyl]phenyl}-1,4,6,7-tetrahydro-5H-pyrazolo[4,3-c]pyridine-5-carboxamide

MS (ESI): mass calcd. for C₃₄H₄₀Cl₂N₆O₂, 634.3; m/z found, 635.2 [M+H]⁺. ¹H NMR (CDCl₃): 7.72 (d, J=2.0, 1H), 7.45-7.35 (m, 4H), 7.24 (d, J=8.6, 2H), 4.80 (br s, 2H), 4.52 (br s, 2H), 4.10-3.85 (m, 5H), 3.75-3.55 (m, 2H), 3.15 (d, J=12, 2H), 2.90-2.50 (m, 6H), 2.28 (d, J=6.6, 2H), 2.10 (t, J=11.2, 1H), 1.82 (t, J=11.2, 1H), 1.70-1.50 (m, 4H), 1.40-1.10 (m, 4H).

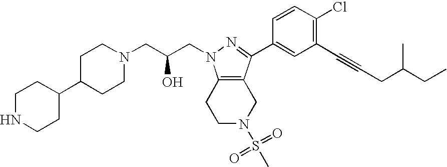

### Example 239

### (2S)-1-(4,4′-Bipiperidin-1-yl)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl}propan-2-ol

MS (ESI): mass calcd. for C₃₂H₄₆ClN₅O₃S, 615.3; m/z found, 616.2 [M+H]⁺. ¹H NMR: 7.62 (s, 1H), 7.40 (s, 2H), 4.40-4.35 (m, 2H), 4.12-3.05 (m, 2H), 3.65-3.50 (m, 4H), 3.35-3.30 (m, 2H), 3.17-3.08 (m, 2H), 3.00-2.80 (m, 4H), 2.85 (s, 3H), 2.30 (d, J=6.5, 2H), 2.18-1.80 (m, 4H), 1.95-1.85 (m, 2H), 1.65-1.55 (m, 4H), 1.40-1.30 (m, 4H), 1.00 (d, J=6.7, 6H).

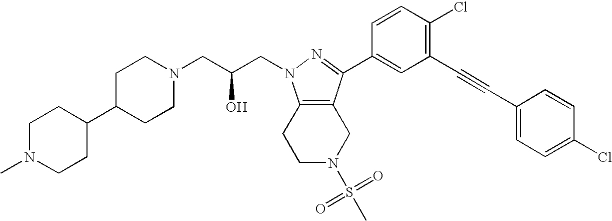

### Example 240

### (2S)-1-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol

The title compound was prepared by methods analogous to those described for Example 74, using formaldehyde (37 wt % in H₂O; 3 equiv.) in dichloroethane. MS (ESI): mass calcd. for C₃₅H₄₃Cl₂N₅O₃S, 683.3; m/z found, 684.2 [M+H]⁺. ¹H NMR (CDCl₃): 7.72 (d, J=1.8, 1H), 7.47-7.38 (m, 4H), 7.28 (d, J=8.7, 2H), 4.50 (dd, J=14.5, 6.4, 2H), 4.10-3.85 (m, 5H), 3.70-3.52 (m, 2H), 3.05-2.80 (m, 4H), 2.80 (s, 3H), 2.30-2.10 (m, 4H), 2.25 (s, 3H), 1.95-1.80 (m, 4H), 1.65-1.55 (m, 4H), 1.40-0.90 (m, 4H).

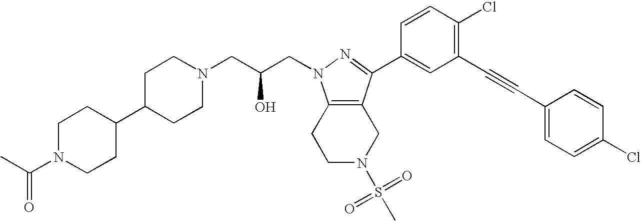

### Example 241

### (2S)-1-(1′-Acetyl-4,4′-bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol

The title compound was prepared using methods analogous to those described for Example 66. MS (ESI): mass calcd. for C₃₆H₄₃Cl₂N₅O₄S, 711.2; m/z found, 712.2 [M+H]⁺. ¹H NMR (CDCl₃): 7.70 (s, 1H), 7.47-7.40 (m, 4H), 7.30-7.25 (m, 2H), 4.52-4.42 (m, 2H), 4.35-4.25 (m, 1H), 4.15-4.10 (m, 1H), 4.05-3.95 (m, 1H), 3.80-3.70 (m, 1H), 3.65-3.55 (m, 2H), 3.30-3.10 (m, 2H), 2.95-2.85 (m, 3H), 2.80 (s, 3H), 2.70-2.60 (m, 2H), 2.40 (t, J=7.6, 2H), 2.30-1.70 (m, 4H), 2.00 (s, 3H), 1.75-1.40 (m, 4H), 1.30-1.00 (m, 4H).

The compounds in Examples 242-248 were prepared using methods analogous to those described for Example 2, using the appropriate alkynes as starting materials.

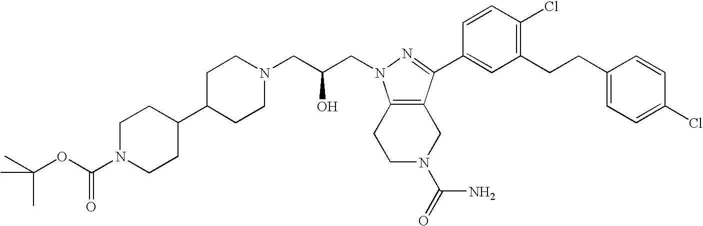

### Example 242

### 1,1-Dimethylethyl 1′-{(2S)-3-[5-(aminocarbonyl)-3-{4-chloro-3-[2-(4-chlorophenyl)ethyl]phenyl}-4,5,6,7-tetrahydro-1H-pyrazolo[4,3-c]pyridin-1-yl]-2-hydroxypropyl}-4,4′-bipiperidine-1-carboxylate

MS (ESI): mass calcd. for C₃₉H₅₂Cl₂N₆O₄, 738.3; m/z found, 739.2 [M+H]⁺. ¹H NMR (CDCl₃): 7.35-7.10 (m, 5H), 7.07 (d, J=7.4, 2H), 4.55 (br s, 2H), 4.40 (br s, 2H), 4.10-3.85 (m, 5H), 3.65-3.50 (m, 2H), 3.05-2.75 (m, 8H), 2.60-2.50 (m, 2H), 2.35-2.30 (d, J=6.5, 2H), 2.15 (t, J=10.3, 1H), 1.90 (t, J=10.5, 1H), 1.65-1.52 (m, 4H), 1.38 (s, 9H), 1.40-1.10 (m, 6H).

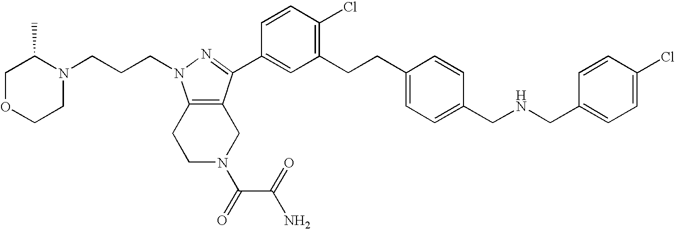

### Example 243

### 2-[3-(4-Chloro-3-{2-[4-({[(4-chlorophenyl)methyl]amino}methyl)phenyl]ethyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide

MS (ESI): mass calcd. for C₃₈H₄₄Cl₂N₆O₃, 702.3; m/z found, 703.2 [M+H]⁺. ¹H NMR (CDCl₃): 7.50-7.15 (m, 11H), 5.65 (br s, 2H), 4.52 (br s, 2H), 4.10-4.00 (m, 2H), 3.70-3.55 (m, 9H), 3.23 (dd, J=11.1, 8.8, 1H), 3.10-3.00 (m, 2H), 2.95-2.90 (m, 2H), 2.80-2.70 (m, 4H), 2.43-2.35 (m, 1H), 2.30-2.20 (m, 2H), 2.10-1.95 (m, 2H), 0.90 (d, J=6.3, 3H).

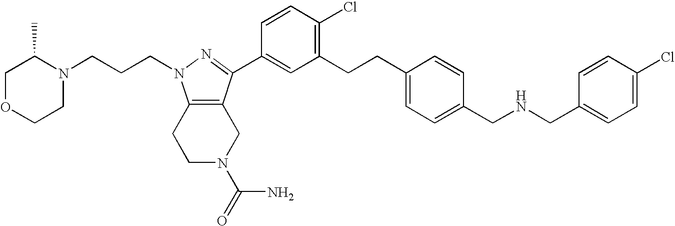

### Example 244

### 3-(4-Chloro-3-{2-[4-({[(4-chlorophenyl)methyl]amino}methyl)phenyl]ethyl}phenyl)-1-{3-[(3S)-3-methylmorpholin-4-yl]propyl}-1,4,6,7-tetrahydro-5H-pyrazolo[4,3-c]pyridine-5-carboxamide

MS (ESI): mass calcd. for C₃₇H₄₄Cl₂N₆O₂, 674.3; m/z found, 675.2 [M+H]⁺. ¹H NMR (CDCl₃): 7.45-7.15 (m, 11H), 4.65 (br s, 2H), 4.52 (br s, 2H), 4.10-4.00 (m, 2H), 3.70-3.55 (m, 9H), 3.23 (dd, J=11.1, 8.8, 1H), 3.10-3.00 (m, 2H), 2.95-2.90 (m, 2H), 2.80-2.70 (m, 4H), 2.43-2.35 (m, 1H), 2.30-2.20 (m, 2H), 2.10-1.95 (m, 2H), 0.90 (d, J=6.3, 3H).

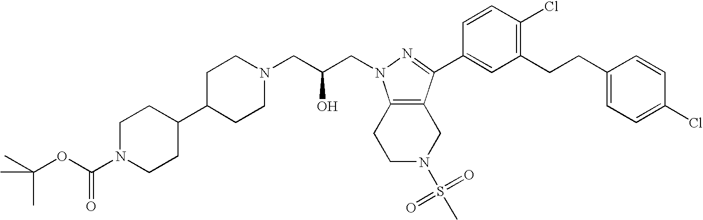

### Example 245

### 1,1-Dimethylethyl 1′-{(2S)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate

MS (ESI): mass calcd. for C₃₉H₅₃Cl₂N₅O₅S, 773.3; m/z found, 774.2 [M+H]⁺. ¹H NMR (CDCl₃): 7.32-7.12 (m, 5H), 7.07 (d, J=7.4, 2H), 4.54 (q, J=14.5, 2H), 4.10-3.85 (m, 5H), 3.65-3.50 (m, 2H), 3.05-2.75 (m, 10H), 2.80 (s, 3H), 2.60-2.50 (m, 2H), 2.32-2.20 (m, 2H), 2.15 (t, J=11.4, 1H), 1.85 (t, J=11.4, 1H), 1.65-1.52 (m, 4H), 1.48 (s, 9H), 1.40-1.10 (m, 4H).

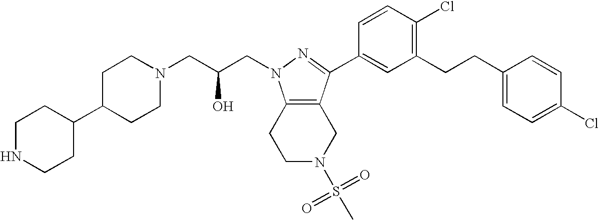

### Example 246

### (2S)-1-(4,4′-Bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol

MS (ESI): mass calcd. for C₃₄H₄₅Cl₂N₅O₃S, 673.3; m/z found, 674.2 [M+H]⁺. ¹H NMR (CDCl₃): 7.35-7.12 (m, 5H), 7.07 (d, J=7.4, 2H), 4.38 (q, J=14.5, 2H), 4.10-3.85 (m, 5H), 3.65-3.50 (m, 2H), 3.05-2.75 (m, 10H), 2.80 (s, 3H), 2.60-2.50 (m, 2H), 2.32-2.20 (m, 2H), 2.12 (t, J=11.4, 1H), 1.85 (t, J=11.4, 1H), 1.65-1.52 (m, 4H), 1.40-1.10 (m, 4H).

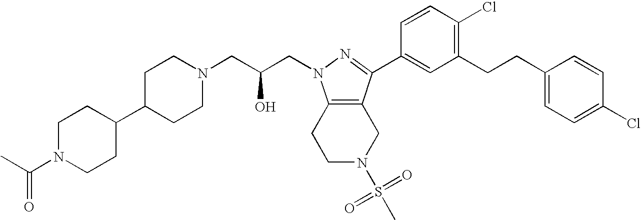

### Example 247

### (2S)-1-(1′-Acetyl-4,4′-bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol

MS (ESI): mass calcd. for C₃₆H₄₇Cl₂N₅O₄S, 715.3; m/z found, 716.2 [M+H]⁺. ¹H NMR (CDCl₃): 7.32-7.15 (m, 5H), 7.05 (d, J=7.4, 2H), 4.60-4.50 (br s, 1H), 4.38 (q, J=14.5, 2H), 4.15-3.90 (m, 5H), 3.80-3.70 (m, 2H), 3.60-3.50 (m, 2H), 3.20-3.05 (m, 2H), 3.05-2.75 (m, 6H), 2.80 (s, 3H), 2.60-2.50 (m, 2H), 2.32-2.20 (m, 4H), 2.00 (s, 3H), 1.70-1.60 (m, 4H), 1.40-1.10 (m, 4H).

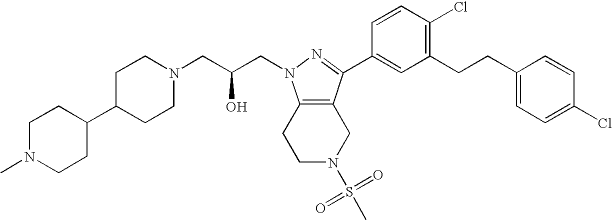

### Example 248

### (2S)-1-[3-{4-Chloro-3-[2-(4-chlorophenyl)ethyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol

MS (ESI): mass calcd. for C₃₅H₄₇Cl₂N₅O₃S, 687.3; m/z found, 688.2 [M+H]⁺. ¹H NMR (CDCl₃): 7.82-7.18 (m, 5H), 7.07 (d, J=7.4, 2H), 4.38 (q, J=14.5, 2H), 4.15-4.00 (m, 2H), 3.92-3.85 (m, 1H), 3.65-3.50 (m, 2H), 3.05-2.75 (m, 9H), 2.80 (s, 3H), 2.32-2.10 (m, 3H), 2.25 (s, 3H), 2.00-1.80 (m, 4H), 1.68-1.58 (m, 4H), 1.40-0.90 (m, 6H).

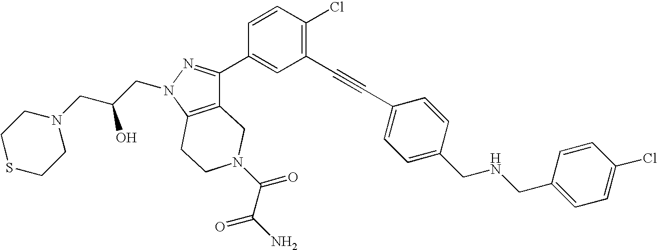

### Example 249

### (S)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide

A. (R)-3-(4-Chloro-3-iodo-phenyl)-1-oxiranylmethyl-1,4,6,7-tetrahydro-pyrazolo[4,3-c]pyridine-5-carboxylic acid tert-butyl ester. (S)-(+)-Glycidyinosylate (17.5 g, 67.5 mmol) was added to a stirring mixture of 3-(4-chloro-3-iodo-phenyl)-1,4,6,7-tetrahydro-pyrazolo[4,3-c]pyridine-5-carboxylic acid tert-butyl ester (15.1 g, 33.0 mmol) and Cs₂CO₃ (21.8 g, 67.0 mmol) in DMF (200 mL) at rt. After 21 h, the mixture was filtered and concentrated to give a crude oil. The oil was diluted with CH₂Cl₂ (150 mL) and washed with satd. aq. Na₂CO₃ (2×75 mL). The organic layer was dried and concentrated to give a gold oil. The oil was purified on SiO₂ (CH₂Cl₂ to 5% acetone/CH₂Cl₂) to provide the desired product (11.9 g, 70%) as a white solid. TLC: R_{f}=0.36 [5% acetone/CH₂Cl₂]. MS (ESI): mass calcd. for C₂₀H₂₃CIIN₃O₃, 515.1; m/z found, 516.0 [M+H]⁺. ¹H NMR (d₆-DMSO): 8.07 (s, 1H), 7.55 (d, J=8.4, 1H), 7.47 (br s, 1H), 4.45 (s, 2H), 4.33 (dd, J=14.8, 3.2, 1H), 4.03 (dd, J=15.2, 6.0, 1H), 3.62-3.48 (m, 2H), 3.23 (br s, 1H), 2.74-2.68 (m, 1H), 2.65 (br s, 2H), 2.49-2.45 (m, 1H), 1.32 (s, 9H).

B. (S)-3-(4-Chloro-3-iodo-phenyl)-1-(2-hydroxy-3-thiomorpholin-4-yl-propyl)-1,4,6,7-tetrahydro-pyrazolo[4,3-c]oyridine-5-carboxylic acid tert-butyl ester. Thiomorpholine (11 mL, 116 mmol) was added to a stirring mixture of (R)-3-(4-chloro-3-iodo-phenyl)-1-oxiranylmethyl-1,4,6,7-tetrahydro-pyrazolo[4,3-c]pyridine-5-carboxylic acid tert-butyl ester (11.8 g, 22.9 mmol) in EtOH (120 mL). After 20 h, the mixture was concentrated to give a golden oil. The oil was purified on SiO₂ (CH₂Cl₂ to 20% acetone/CH₂Cl₂) to give the desired product (13.7 g, 97%) as a white solid. TLC: R_{f}=0.46 [15% acetone/CH₂Cl₂]. MS (ESI): mass calcd. for C₂₄H₃₂CIIN₄O₃S, 618.1; m/z found, 619.1 [M+H]⁺. ¹H NMR (d₆-DMSO): 7.97 (s, 1H), 7.46 (d, J=8.0, 1H), 7.38 (br s, 1H), 4.72 (d, J=4.8, 1H), 4.36 (s, 2H), 3.98-3.92 (m, 1H), 3.89-3.71 (m, 2H), 3.45 (br s, 2H), 2.58 (d, J=4.4, 2H), 2.51 (d, J=4.8, 4H), 2.42-2.35 (m, 4H), 2.18 (t, J=6.0, 2H), 1.24 (s, 9H).

C. 1-[3-(4-Chloro-3-iodo-phenyl)-4,5,6,7-tetrahydro-pyrazolo[4,3-c]pyridin-1-yl-3-thiomorpholin-4-yl-propan-2-ol. Trifluoroacetic acid (30 mL) was added to a stirring solution of (S)-3-(4-chloro-3-iodo-phenyl)-1-(2-hydroxy-3-thiomorpholin-4-yl-propyl)-1,4,6,7-tetrahydro-pyrazolo[4,3-c]pyridine-5-carboxylic acid tert-butyl ester (13.6 g, 22.0 mmol) in CH₂Cl₂ (30 mL). After 2 h, the mixture was concentrated to give a golden liquid. The liquid was set stirring in CH₂Cl₂ (200 mL) and satd. aq. K₂CO₃ (200 mL) was added slowly over 15 min. The organic layer was separated, dried, and concentrated to give the desired product (9.3 g, 82%) as a white solid. MS (ESI): mass calcd. for C₁₉H₂₄CIIN₄OS, 518.0; m/z found, 519.0 [M+H]⁺. ¹H NMR (d₆-DMSO): 8.34 (s, 1H), 7.79 (d, J=8.4, 1H), 7.74 (dd, J=8.4, 2.0, 1H), 5.06 (d, J=4.8, 1H), 4.28 (dd, J=13.6, 3.6, 1H), 4.27-4.23 (m, 1H), 4.22-4.07 (m, 1H), 4.05 (s, 2H), 3.12 (br s, 2H), 2.95-2.73 (m, 11H), 2.54 (t, J=6.0, 2H).

D. (S)-2-[3-(4-Chloro-3-iodo-phenyl)-1-(2-hydroxy-3-thiomorpholin-4-yl-propyl)-1,4,6,7-tetrahydro-pyrazolo[4,3-c]pyridin-5-yl]-2-oxo-acetamide. Oxamic acid (1.76 g, 19.8 mmol) was added to a stirring mixture of 1,1′-carbonyldiimidazole (CDI) (3.19 g, 19.7 mmol) in DMF (50 mL). After 75 min, a solution of 1-[3-(4-chloro-3-iodo-phenyl)-4,5,6,7-tetrahydro-pyrazolo[4,3-c]pyridin-1-yl]-3-thiomorpholin-4-yl-propan-2-ol (5.3 g, 10.2 mmol) in DMF (50 mL) was added and the mixture was stirred at rt. After 15 min, the mixture was concentrated to give a cloudy yellow liquid. The liquid was diluted with EtOAc (200 mL) and washed twice with H₂O (200 mL). The organic layer was dried and concentrated to give the desired product (6.0 g, 100%) as a white solid. MS (ESI): mass calcd. for C₂₁H₂₅CIIN₅O₃S, 589.0; m/z found, 590.0 [M+H]⁺. ¹H NMR (d₆-DMSO): 8.30-8.25 (m, 2H), 8.21-7.90 (m, 2H), 7.88-7.50 (m, 1H), 5.06-4.75 (m, 3H), 4.28-4.00 (m, 2H), 3.97-3.78 (m, 2H), 3.05-2.75 (m, 11H), 2.62-2.42 (m, 2H).

E. (4-Chloro-benzyl)-(4-ethynyl-benzyl)-amine. 4-Trimethylsilanylethynyl-benzaldehyde (commercially available or available via Sonogashira coupling; 1.0 g, 4.9 mmol, 1.0 equiv) was dissolved in MeOH (20 mL) and 4-chlorobenzylamine (0.74 g, 5.2 mmol, 1.05 equiv) was added. After stirring at rt for 16 h, NaBH₄ (0.19 g, 4.9 mmol, 1.0 equiv) was added slowly. 1 N HCl was slowly added to quench the unreacted NaBH₄. The mixture was concentrated and the residue was partitioned between CH₂Cl₂ and satd. aq. NaHCO₃. The organic layer was dried and concentrated. The crude compound was purified (SiO₂; EtOAc/hexanes) to provide the title compound (1.1 g, 4.3 mmol, 90%). TLC: R_{f}=0.54 (50% EtOAc/hexanes). Alternatively, the material was converted to the corresponding HCl salt and purified by recrystallization.

F. (S)-2-[3-(4-Chloro-3-[4-[(4-chloro-benzylamino)-methyl]-phenylethynyl]-phenyl)-1-(2-hydroxy-3-thiomorpholin-4-yl-propyl)-1,4,6,7-tetrahydro-pyrazolo[4,3-c]pyridin-5-yl]-2-oxo-acetamide. Triethylamine (1.5 mL, 10.8 mmol) was added to a stirring mixture of (S)-2-[3-(4-chloro-3-iodo-phenyl)-1-(2-hydroxy-3-thiomorpholin-4-yl-propyl)-1,4,6,7-tetrahydro-pyrazolo[4,3-c]pyridin-5-yl]-2-oxo-acetamide (2.1 g, 3.5 mmol) and (4-chloro-benzyl)-(4-ethynyl-benzyl)-amine (1.5 g, 6.0 mmol) in DMF (15 mL). The flask was equipped with a nitrogen inlet, septum, and vacuum line. The mixture was evacuated and flushed with nitrogen three times. To the mixture was added Pd(PPh₃)₂Cl₂ (51 mg, 0.073 mmol) and CuI (36 mg, 0.19 mmol) in one portion while keeping the contents under a positive pressure of nitrogen. After 3 h, the mixture was added to H₂O (100 mL) and EtOAc (100 mL). The organic layer was separated, washed once with H₂O (50 mL), dried, and concentrated to give a golden brown oil. The oil was purified on SiO₂ (CH₂Cl₂ to 100% (5% MeOH(2 M NH₃)/CH₂Cl₂)) to give the desired product (1.72 g, 68%) as a light yellow solid. TLC: R_{f}=0.42 [10% MeOH(2 M NH₃)/CH₂Cl₂]. MS (ESI): mass calcd. for C₃₇H₃₈Cl₂N₆O₃S, 716.2; m/z found, 717.2 [M+H]⁺. ¹H NMR (d₆-DMSO): 8.00-7.88 (m, 2H), 7.82-7.75 (m, 2H), 7.74-7.65 (m, 2H), 7.55 (d, J=8.4, 2H), 7.50 (s, 4H), 5.08-5.00 (m, 1H), 4.89-4.83 (m, 2H), 4.30-4.23 (m, 1H), 4.21-4.07 (m, 2H), 3.95-3.72 (m, 6H), 3.12-2.91 (m, 4H), 2.90-2.80 (m, 4H), 2.79-2.69 (m, 4H), 2.54-2.47 (m, 2H).

The compounds in Examples 250-275 were prepared using methods analogous to those described for Example 249, with exceptions as noted.

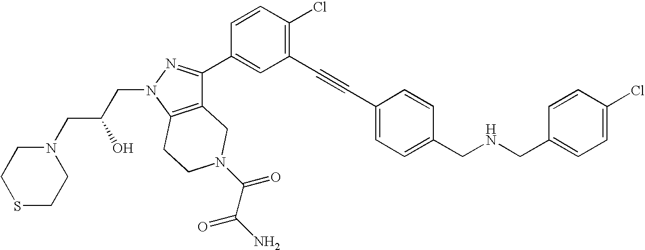

### Example 250

### (R)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide

MS (ESI): mass calcd. for C₃₇H₃₈Cl₂N₆O₃S, 716.2; m/z found, 717.2 [M+H]⁺. ¹H NMR (d₆-DMSO): 8.00-7.88 (m, 2H), 7.82-7.75 (m, 2H), 7.74-7.65 (m, 2H), 7.55 (d, J=8.4, 2H), 7.50 (s, 4H), 5.08-5.00 (m, 1H), 4.89-4.83 (m, 2H), 4.30-4.23 (m, 1H), 4.21-4.07 (m, 2H), 3.95-3.72 (m, 6H), 3.12-2.91 (m, 4H), 2.90-2.80 (m, 4H), 2.79-2.69 (m, 4H), 2.54-2.47 (m, 2H).

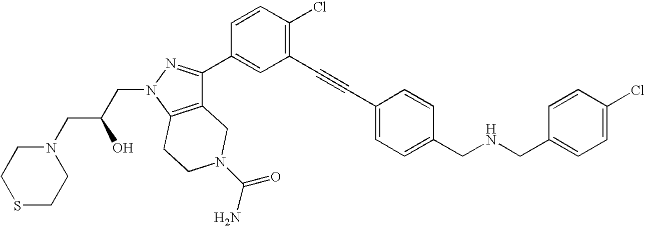

### Example 251

### (S)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide

MS (ESI): mass calcd. for C₃₆H₃₈Cl₂N₆O₂S, 688.2; m/z found, 689.2 [M+H]⁺. ¹H NMR (d₆-DMSO): 10.11 (s, 1H), 9.69 (s, 2H), 7.83 (s, 1H), 7.65-7.48 (m, 8H), 7.42 (d, J=8.4, 2H), 4.54-4.44 (m, 6H), 4.18-4.00 (m, 5H), 3.68-3.52 (m, 4H), 3.35-3.25 (m, 1H), 3.24-3.00 (m, 6H), 2.78-2.60 (m, 4H).

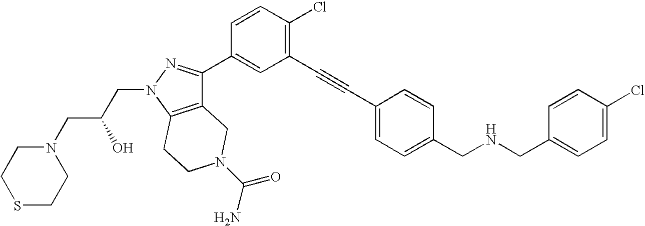

### Example 252

### (R)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide

MS (ESI): mass calcd. for C₃₆H₃₈Cl₂N₆O₂S, 688.2; m/z found, 689.2 [M+H]⁺. ¹H NMR (d₆-DMSO): 10.11 (s, 1H), 9.69 (s, 2H), 7.83 (s, 1H), 7.65-7.48 (m, 8H), 7.42 (d, J=8.4, 2H), 4.54-4.44 (m, 6H), 4.18-4.00 (m, 5H), 3.68-3.52 (m, 4H), 3.35-3.25 (m, 1H), 3.24-3.00 (m, 6H), 2.78-2.60 (m, 4H).

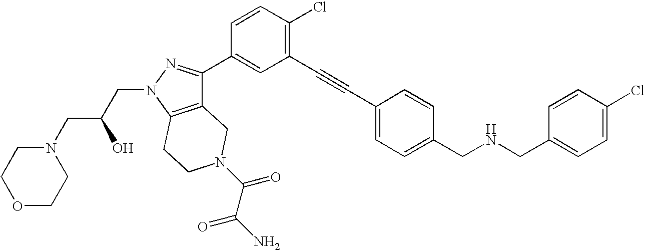

### Example 253

### (S)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide

MS (ESI): mass calculated for C₃₇H₃₈Cl₂N₆O₄, 700.2; m/z found, 701.2 [M+H]⁺. ¹H NMR (d₆-DMSO) 8.16 (s, 1H), 7.90-7.75 (m, 2H), 7.73-7.50 (m, 4H), 7.43 (d, J=8.4, 2H), 7.38 (s, 4H), 5.00-4.95 (m, 1H), 4.80-4.70 (m, 2H), 4.25-4.15 (m, 1H), 4.13-3.95 (m, 2H), 3.94-3.65 (m, 6H), 3.59-3.50 (br s, 4H), 2.90-2.80 (m, 3H), 2.48-2.30 (m, 6H).

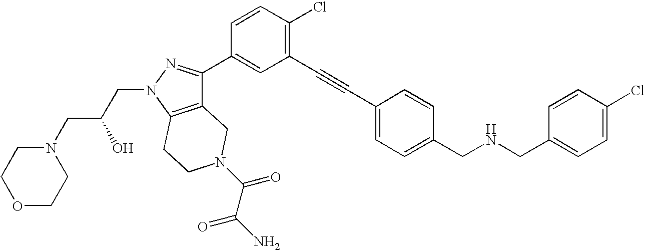

### Example 254

### (R)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide

MS (ESI): mass calcd. for C₃₇H₃₈Cl₂N₆O₄, 700.2; m/z found, 701.2 [M+H]⁺. ¹H NMR (d₆-DMSO): 8.16 (s, 1H), 7.90-7.75 (m, 2H), 7.73-7.50 (m, 4H), 7.43 (d, J=8.4, 2H), 7.38 (s, 4H), 5.00-4.95 (m, 1H), 4.80-4.70 (m, 2H), 4.25-4.15 (m, 1H), 4.13-3.95 (m, 2H), 3.94-3.65 (m, 6H), 3.59-3.50 (br s, 4H), 2.90-2.80 (m, 3H), 2.48-2.30 (m, 6H).

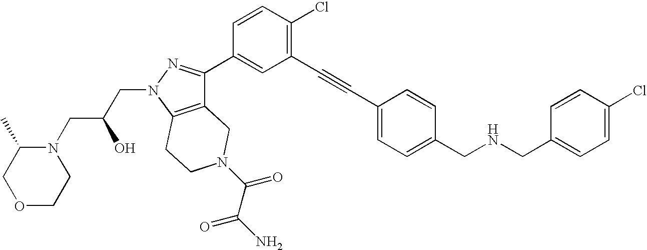

### Example 255

### 2-{3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₈H₄₀Cl₂N₆O₄, 714.3; m/z found, 715.2 [M+H]⁺. ¹H NMR (d₆-DMSO): 12.40 (br s, 1H), 10.91 (br s, 2H), 9.16 (s, 1H), 8.90-8.35 (m, 10H), 8.28 (d, J=9.8, 2H), 7.38 (s, 4H), 5.05-5.00 (m, 1H), 4.70-4.60 (m, 1H), 4.50-4.35 (m, 6H), 4.32-3.85 (m, 5H), 3.82-3.65 (m, 3H), 3.50-3.15 m, 2H), 3.10-2.90 (m, 2H), 1.06 (d, J=7.6, 2H).

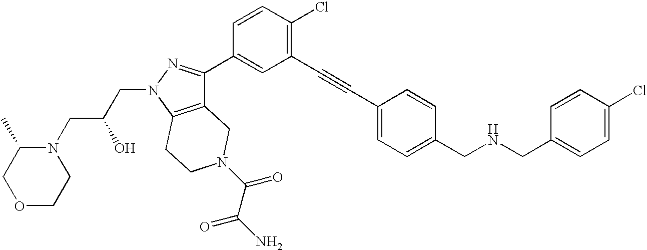

### Example 256

### 2-{3-(4-Chloro-3-{4-[(4-chloro-benzylamino)-methyl]-phenylethynyl}-phenyl)-1-[(2R)-2-hydroxy-3-((3S)-3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₈H₄₀Cl₂N₆O₄, 714.3; m/z found, 715.2 [M+H]⁺. ¹H NMR (d₆-DMSO): 12.40 (br s, 1H), 10.91 (br s, 2H), 9.16 (s, 1H), 8.90-8.35 (m, 10H), 8.28 (d, J=9.8, 2H), 7.38 (s, 4H), 5.05-5.00 (m, 1H), 4.70-4.60 (m, 1H), 4.50-4.35 (m, 6H), 4.32-3.85 (m, 5H), 3.82-3.65 (m, 3H), 3.50-3.15 m, 2H), 3.10-2.90 (m, 2H), 1.06 (d, J=7.6, 2H).

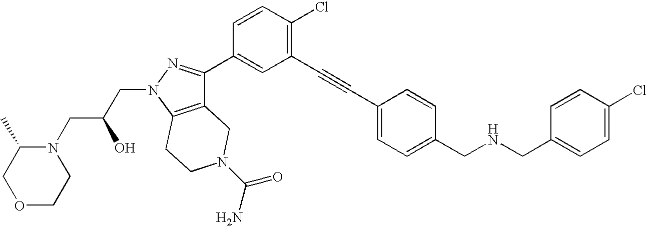

### Example 257

### 3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide

MS (ESI): mass calcd. for C₃₇H₄₀Cl₂N₆O₃, 686.3; m/z found, 687.2 [M+H]⁺. ¹H NMR (d₆-DMSO): 12.30 (br s, 1H), 10.83 (br s, 2H), 8.73 (s, 1H), 8.65-8.38 (m, 10H), 8.27 (d, J=7.6, 2H), 4.98-4.35 (m, 8H), 4.25-3.95 (m, 4H), 3.92-3.55 (m, 7H), 3.50-3.15 m, 3H), 2.79 (s, 2H), 1.05 (d, J=7.8, 2H).

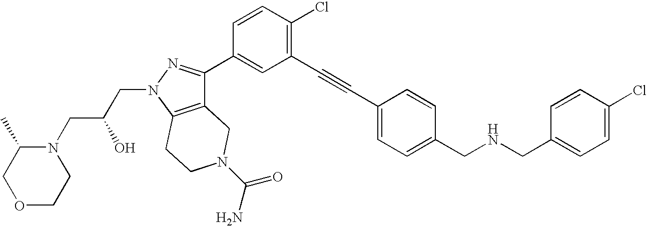

### Example 258

### 3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide

MS (ESI): mass calcd. for C₃₇H₄₀Cl₂N₆O₃, 686.3; m/z found, 687.2 [M+H]⁺. ¹H NMR (d₆-DMSO): 12.30 (br s, 1H), 10.83 (br s, 2H), 8.73 (s, 1H), 8.65-8.38 (m, 10H), 8.27 (d, J=7.6, 2H), 4.98-4.35 (m, 8H), 4.25-3.95 (m, 4H), 3.92-3.55 (m, 7H), 3.50-3.15 m, 3H), 2.79 (s, 2H), 1.05 (d, J=7.8, 2H).

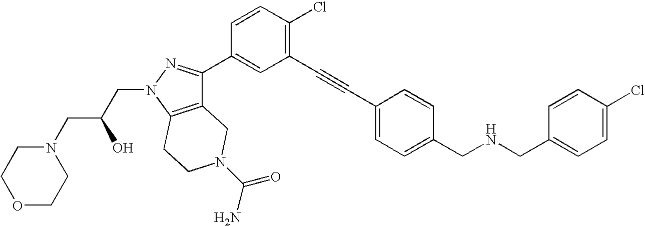

### Example 259

### (S)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide

MS (ESI): mass calcd. for C₃₆H₃₈Cl₂N₆O₃, 672.2; m/z found, 673.2 [M+H]⁺. ¹H NMR (d₆-DMSO): 10.30 (br s, 1H), 9.77 (br s, 2H), 7.92 (s, 1H), 7.80-7.55 (m, 10H), 7.52 (d, J=6.6, 2H), 4.58 (s, 2H), 4.40 (br s, 1H), 4.25-3.90 (m, 9H), 3.89-3.73 (m, 2H), 3.70-3.58 (br s, 2H), 3.56-3.38 (m, 3H), 3.16 (br s, 3H), 2.77 (s, 2H).

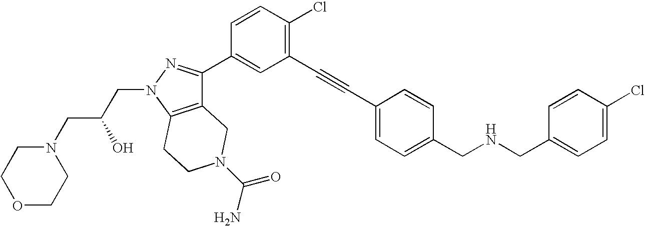

### Example 260

### (R)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide

MS (ESI): mass calcd. for C₃₆H₃₈Cl₂N₆O₃, 672.2; m/z found, 673.2 [M+H]⁺. ¹H NMR (d₆-DMSO): 10.30 (br s, 1H), 9.77 (br s, 2H), 7.92 (s, 1H), 7.80-7.55 (m, 10H), 7.52 (d, J=6.6, 2H), 4.58 (s, 2H), 4.40 (br s, 1H), 4.25-3.90 (m, 9H), 3.89-3.73 (m, 2H), 3.70-3.58 (br s, 2H), 3.56-3.38 (m, 3H), 3.16 (br s, 3H), 2.77 (s, 2H).

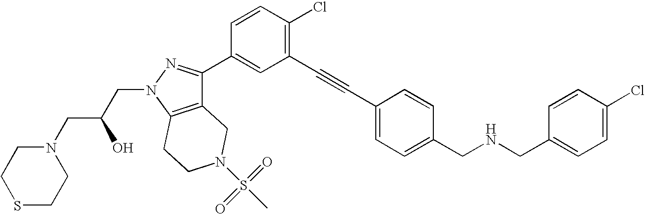

### Example 261

### (S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol

MS (ESI): mass calcd. for C₃₆H₃₉Cl₂N₅O₃S₂, 723.2; m/z found, 724.2 [M+H]⁺. ¹H NMR (d₆-DMSO): 10.23 (br s, 1H), 9.78 (br s, 2H), 7.90 (s, 1H), 7.75-7.58 (m, 8H), 7.51 (d, J=8.4, 2H), 4.46 (s, 2H), 4.30-4.05 (m, 6H), 3.82-3.68 (m, 2H), 3.55-3.45 (m, 2H), 3.40-3.32 (m, 1H), 3.25-3.00 (m, 10H), 2.93 (br s, 2H), 2.86-2.76 (m, 2H).

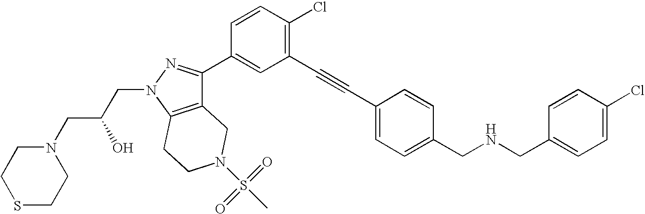

### Example 262

### (R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol

MS (ESI): mass calcd. for C₃₆H₃₉Cl₂N₅O₃S₂, 723.2; m/z found, 724.2 [M+H]⁺. ¹H NMR (d₆-DMSO): 10.23 (br s, 1H), 9.78 (br s, 2H), 7.90 (s, 1H), 7.75-7.58 (m, 8H), 7.51 (d, J=8.4, 2H), 4.46 (s, 2H), 4.30-4.05 (m, 6H), 3.82-3.68 (m, 2H), 3.55-3.45 (m, 2H), 3.40-3.32 (m, 1H), 3.25-3.00 (m, 10H), 2.93 (br s, 2H), 2.86-2.76 (m, 2H).

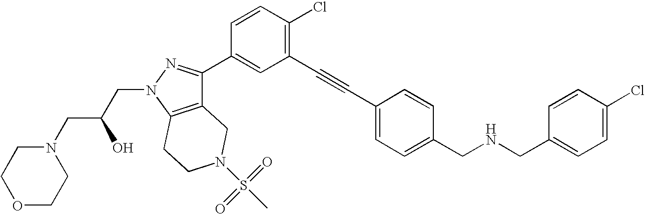

### Example 263

### (S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol

MS (ESI): mass calcd. for C₃₆H₃₉Cl₂N₅O₄S, 707.2; m/z found, 708.1 [M+H]⁺. ¹H NMR (d₆-DMSO): 10.28 (br s, 1H), 9.71 (br s, 2H), 7.91 (s, 1H), 7.74-7.57 (m, 8H), 7.52 (d, J=6.5, 2H), 4.47 (s, 3H), 4.28-4.06 (m, 6H), 3.98-3.90 (m, 2H), 3.89-3.27 (m, 10H), 3.23-3.05 (m, 4H), 2.94 (br s, 2H).

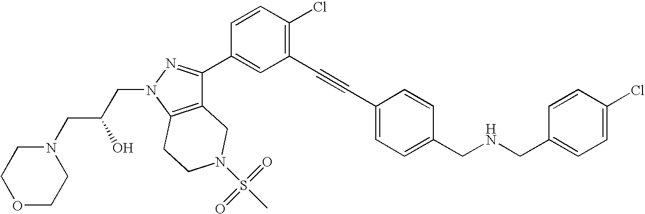

### Example 264

### (R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol

MS (ESI): mass calcd. for C₃₆H₃₉Cl₂N₅O₄S, 707.2; m/z found, 708.1 [M+H]⁺. ¹H NMR (d₆-DMSO): 10.28 (br s, 1H), 9.71 (br s, 2H), 7.91 (s, 1H), 7.74-7.57 (m, 8H), 7.52 (d, J=6.5, 2H), 4.47 (s, 3H), 4.28-4.06 (m, 6H), 3.98-3.90 (m, 2H), 3.89-3.27 (m, 10H), 3.23-3.05 (m, 4H), 2.94 (br s, 2H).

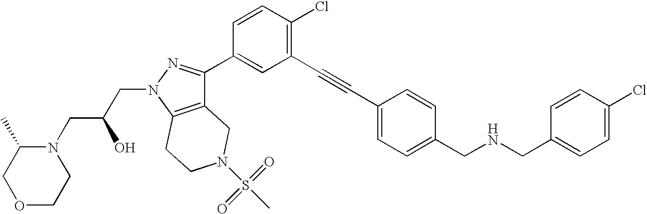

### Example 265

### (2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol

MS (ESI): mass calcd. for C₃₇H₄₁Cl₂N₅O₄S, 721.2; m/z found, 722.2 [M+H]⁺. ¹H NMR (d₆-DMSO): 12.28 (br s, 1H), 10.81 (br s, 2H), 8.73 (s, 1H), 8.74-8.57 (m, 8H), 8.52 (d, J=9.8, 2H), 4.76 (s, 3H), 4.48-4.34 (m, 6H), 4.25-4.00 (m, 3H), 3.91-3.67 (m, 5H), 3.50-2.95 (m, 10H), 1.02 (d, J=7.2, 2H).

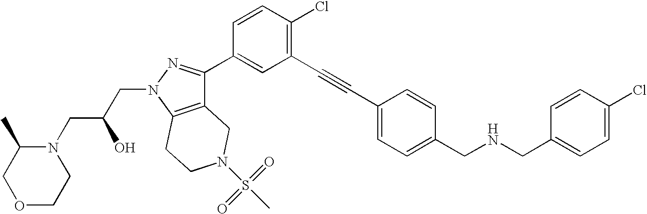

### Example 266

### (2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol

MS (ESI): mass calcd. for C₃₇H₄₁Cl₂N₅O₄S, 721.2; m/z found, 722.3 [M+H]⁺. ¹H NMR (d₆-DMSO): 9.81 (br s, 1H), 9.69 (br s, 2H), 7.82 (s, 1H), 7.72-7.52 (m, 8H), 7.43 (d, J=6.4, 2H), 4.39 (s, 1H), 4.32 (br s, 2H), 4.20-4.01 (m, 6H), 4.00-3.62 (m, 9H), 3.55-3.25 (m, 5H), 3.18-2.95 (m, 2H), 2.86 (br s, 2H), 1.10 (d, J=7.2, 2H).

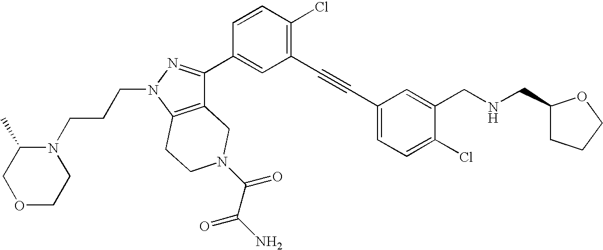

### Example 267

### 2-{3-[4-Chloro-3-(4-chloro-3-{[((2S)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₆H₄₂Cl₂N₆O₄, 692.3; m/z found, 693.2 [M+H]⁺. ¹H NMR (d₆-DMSO): 11.08 (br s, 1H), 9.18 (br s, 2H), 8.06 (s, 1H), 7.92-7.68 (m, 3H), 7.61-7.45 (m, 4H), 4.65 (s, 2H), 4.32-4.17 (m, 2H), 4.16-3.98 (m, 3H), 3.92-3.53 (m, 7H), 3.20-2.90 (m, 5H), 2.89-2.68 (m, 3H), 2.25-2.00 (br s, 2H), 1.98-1.82 (m, 1H), 1.78-1.68 (m, 2H), 1.58-1.45 (m, 1H), 1.23-1.05 (m, 5H).

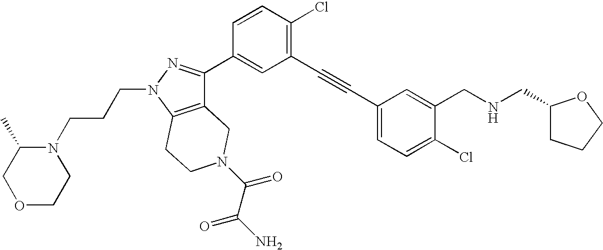

### Example 268

### 2-{3-[4-Chloro-3-(4-chloro-3-{[((2R)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₆H₄₂Cl₂N₆O₄, 692.3; m/z found, 693.2 [M+H]⁺. ¹H NMR (d₆-DMSO): 11.08 (br s, 1H), 9.18 (br s, 2H), 8.06 (s, 1H), 7.92-7.68 (m, 3H), 7.61-7.45 (m, 4H), 4.65 (s, 2H), 4.32-4.17 (m, 2H), 4.16-3.98 (m, 3H), 3.92-3.53 (m, 7H), 3.20-2.90 (m, 5H), 2.89-2.68 (m, 3H), 2.25-2.00 (br s, 2H), 1.98-1.82 (m, 1H), 1.78-1.68 (m, 2H), 1.58-1.45 (m, 1H), 1.23-1.05 (m, 5H).

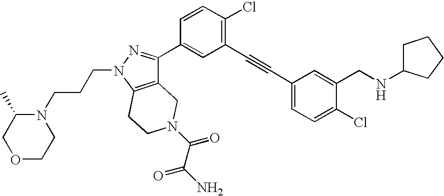

### Example 269

### (S)-2-{3-[4-Chloro-3-(4-chloro-3-cyclopentylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide

A. 2-Chloro-5-iodo-benzoyl chloride. A stirring solution of 2-chloro-5-iodo-benzoic acid (12.4 g, 43.9 mmol) in SOCl₂ (40 mL, 550 mmol) was heated to reflux under a N₂ atmosphere. After 20 h, the mixture was concentrated to give the product (13.2 g, 100%) as a yellow solid. ¹H NMR (d₆-DMSO): 7.88 (s, 1H), 7.65 (dd, J=8.4, 2.0, 1H), 7.14 (d, J=8.4, 1H).

B. 2-Chloro-N-cyclopentyl-5-iodo-benzamide. 2-Chloro-5-iodo-benzoyl chloride (4.1 g, 13.6 mmol) was added to a stirring solution of cyclopentylamine (4.0 mL, 40.5 mmol) in THF (50 mL) at rt. After 1 h, the mixture was concentrated, and the residue was diluted with CH₂Cl₂ (100 mL) and washed with 0.5 M HCl. The organic layer was dried and concentrated to give the desired product (4.5 g, 94%) as a white solid. MS (ESI): mass calcd. for C₁₂H₁₃ClINO, 349.0; m/z found, 350.0 [M+H]⁺. ¹H NMR (d₆-DMSO): 8.22 (d, J=7.2, 1H), 7.52 (d, J=8.4, 1H), 7.4 (s, 1H), 7.04 (d, J=8.4, 1H), 3.98-3.80 (m, 1H), 1.69-1.55 (m, 2H), 1.48-1.38 (m, 2H), 1.37-1.18 (m, 4H).

C. (2-Chloro-5-iodo-benzyl)-cyclopentyl-amine. Borane dimethyl sulfide (3.5 mL, 76.0 mmol) was added via syringe over 5 min to a stirring solution of 2-chloro-N-cyclopentyl-5-iodo-benzamide (4.3 g, 12.3 mmol) in THF (75 mL). Once addition was complete the mixture was heated to 70° C. After 3 h, the mixture was cooled to rt and MeOH (10 mL) was added slowly over 10 min. Once addition of MeOH was complete, 1 M NaOH (10 mL) was added and the mixture was heated to 70° C. After 2 h, the mixture was cooled to rt and concentrated to give a golden oil. The oil was diluted with CH₂Cl₂ (100 mL) and washed with H₂O (75 mL). The organic layer was dried and concentrated to give a clear golden oil. The oil was purified on SiO₂ (CH₂Cl₂ to 5% acetone/CH₂Cl₂) to provide a colorless oil. The oil was diluted with 1:1 CH₂Cl₂/Et₂O (25 mL) and treated with 1 M HCl/Et₂O. The mixture was concentrated to give the desired product (3.3 g, 72%) as a white solid. MS (ESI): mass calcd. for C₁₂H₁₅ClIN, 335.0; m/z found, 336.0 [M+H]⁺. ¹H NMR (d₆-DMSO): 9.39 (s, 2H), 8.18 (d, J=2.1, 1H), 7.80 (dd, J=8.4, 2.2, 1H), 7.36 (s, J=8.4, 1H), 4.20 (t, J=6.2, 2H), 3.62-3.46 (m, 2H), 2.10-1.96 (m, 2H), 1.80-1.68 (m, 3H), 1.62-1.46 (m, 2H).

D. The title compound was prepared using methods analogous to those described for Example 249. MS (ESI): mass calcd. for C₃₆H₄₂Cl₂N₆O₃, 676.3; m/z found, 677.3 [M+H]⁺. ¹H NMR (d₆-DMSO): 11.15 (br s, 1H), 9.24 (br s, 2H), 8.17 (s, 1H), 8.05-7.80 (m, 3H), 7.77-7.50 (m, 4H), 4.75 (s, 2H), 4.32-4.17 (m, 2H), 4.00-3.68 (m, 6H), 3.67-3.53 (m, 2H), 3.52-3.32 (m, 2H), 3.31-3.05 (m, 2H), 2.98-2.75 (br s, 2H), 2.35-2.15 (m, 2H), 2.12-1.98 (m, 2H), 1.85-1.48 (m, 6H), 1.24 (dd, J=12.4, 6.4, 5H).

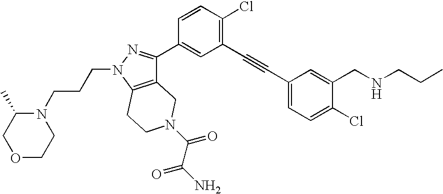

### Example 270

### (S)-2-{3-[4-Chloro-3-(4-chloro-3-propylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₄H₄₀Cl₂N₆O₃, 650.3; m/z found, 651.3 [M+H]⁺. ¹H NMR (d₆-DMSO): 10.95 (br s, 1H), 9.05 (br s, 2H), 8.07 (s, 1H), 7.92 (s, 1H), 7.85-7.68 (m, 2H), 7.72-7.45 (m, 4H), 4.65 (s, 2H), 4.35-3.98 (m, 4H), 3.95-3.58 (m, 3H), 3.55-3.20 (m, 4H), 3.18-2.68 (m, 7H), 2.25-2.09 (br s, 2H), 1.70-1.50 (m, 2H), 1.19 (dd, J=20.8, 6.4, 4H), 0.86 (t, J=7.6, 3H).

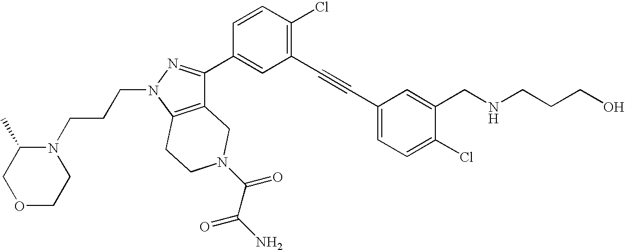

### Example 271

### (S)-2-{3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₄H₄₀Cl₂N₆O₄, 666.3; m/z found, 667.2 [M+H]⁺. ¹H NMR (d₆-DMSO): 11.08 (br s, 1H), 9.08 (br s, 2H), 8.08 (s, 1H), 7.94 (s, 1H), 7.87-7.68 (m, 2H), 7.67-7.43 (m, 4H), 4.78-4.58 (m, 3H), 4.25 (s, 2H), 4.09 (s, 3H), 3.95-3.58 (m, 7H), 3.44 (t, J=5.6, 3H), 3.02 (s, 4H), 2.77 (br s, 2H), 2.30-2.05 (br s, 2H), 1.90-1.69 (m, 2H), 1.12 (br s, 3H).

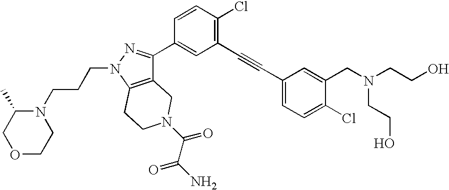

### Example 272

### (S)-2-{3-[3-(3-{[Bis-(2-hydroxy-ethyl)-amino]-methyl}-4-chloro-phenylethynyl)-4-chloro-phenyl]-1-[3-(3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₅H₄₂Cl₂N₆O₅, 696.3; m/z found, 697.4 [M+H]⁺. ¹H NMR (d₆-DMSO): 11.08 (br s, 1H), 9.57 (br s, 1H), 8.25-8.05 (m, 2H), 7.94-7.80 (m, 2H), 7.79-7.50 (m, 4H), 5.39 (br s, 2H), 4.73 (s, 2H), 4.65 (s, 2H), 4.25-4.05 (s, 2H), 4.00-3.62 (m, 10H), 3.56-3.46 (m, 2H), 3.22 (s, 3H), 3.07 (br s, 3H), 2.95-2.75 (m, 2H), 2.22 (br s, 2H), 1.24 (d, J=6.8, 4H).

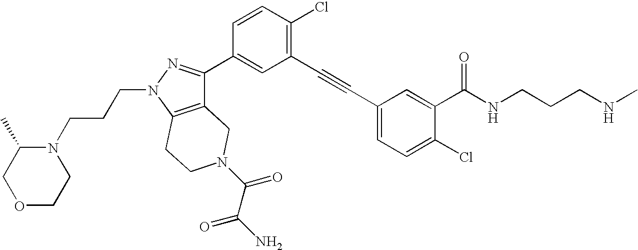

### Example 273

### (S)-5-(5-{5-Aminooxalyl-1-[3-(3-methyl-morpholin-4-yl)-propyl]-4,5,6,7-tetrahydro-1H-pyrazolo[4,3-c]pyridin-3-yl}-2-chloro-phenylethynyl)-2-chloro-N-(3-methylamino-propyl)-benzamide

MS (ESI): mass calcd. for C₃₅H₄₁Cl₂N₇O₄, 693.3; m/z found, 694.2 [M+H]⁺. ¹H NMR (d₆-DMSO): 10.71 (br s, 1H), 8.51 (t, J=5.6, 1H), 8.38 (br s, 2H), 7.94 (br s, 1H), 7.69 (s, 1H), 7.65-7.55 (m, 2H), 7.54-7.32 (m, 4H), 4.58-4.40 (m, 2H), 4.05-3.88 (m, 2H), 3.78-3.05 (s, 11H), 3.02-2.55 (m, 6H), 2.33 (t, J=5.2, 2H), 2.12-1.92 (m, 2H), 1.70-1.50 (m, 2H), 1.04 (d, J=6.8, 4H).

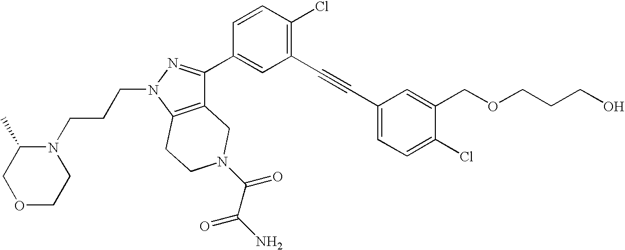

### Example 274

### (S)-2-{3-{4-Chloro-3-[4-chloro-3-(3-hydroxy-propoxymethyl)-phenylethynyl]-phenyl}-1-[3-(3-methyl-morpholin-4-yl)-propyl]-1,4,6,7-tetrahydro-pyrazolo[4,3-c]pyridin-5-yl}-2-oxo-acetamide

A. 3-(2-Chloro-5-iodo-benzyloxy)-propan-1-ol. 1,3-Propanediol (700 μL, 9.7 mmol) was added to a stirring mixture of NaH (60% in oil; 0.45 g, 11.3 mmol) and 2-bromomethyl-1-chloro-4-iodo-benzene (2.6 g, 7.7 mmol) in THF (20 mL). The mixture was heated at reflux under a N₂ atmosphere for 20 h. The mixture was cooled to rt, diluted with satd. aq. NH₄Cl and extracted with CH₂Cl₂ (2×25 mL). The organic layers were combined, dried, and concentrated to give a golden oil. The oil was purified on SiO₂ (CH₂Cl₂ to 10% acetone/CH₂Cl₂) to give the desired product (1.5 g, 60%) as a clear light golden oil. TLC: R_{f}=0.17 [CH₂Cl₂]. MS (ESI): mass calcd. C₁₀H₁₂ClIO₂, 326.0; m/z found, 349.0 [M+Na]⁺. ¹H NMR (d₆-DMSO): 7.80 (d, J=2.2, 1H), 7.66 (dd, J=8.4, 2.2, 1H), 7.25 (d, J=8.3, 1H), 4.48 (s, 2H), 4.46 (t, J=5.2, 1H), 3.57 (t, J=6.4, 2H), 3.49 (q, J=6.3, 2H), 1.72 (p, J=6.4, 2H).

B. The title compound was prepared using methods analogous to those described for Example 249. MS (ESI): mass calcd. for C₃₄H₃₉Cl₂N₅O₅, 667.2; m/z found, 668.2 [M+H]⁺. ¹H NMR (d₆-DMSO): 8.09 (s, 1H), 7.88-7.68 (m, 2H), 7.65-7.54 (m, 3H), 7.52-7.42 (m, 2H), 4.67 (s, 2H), 4.49 (s, 2H), 4.38 (t, J=5.2, 2H), 3.99 (t, J=6.8, 2H), 3.78-3.68 (m, 2H), 3.67-3.32 (m, 7H), 3.08-2.98 (m, 1H), 2.90-2.70 (m, 2H), 2.68-2.55 (m, 2H), 2.32-2.14 (m, 2H), 1.98-1.75 (m, 2H), 1.67 (p, J=6.4, 2H), 0.77 (d, J=6.0, 3H).

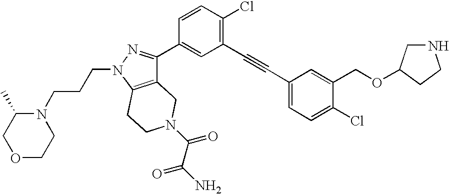

### Example 275

### (S)-2-[3-[4-Chloro-3-[4-chloro-3-(pyrrolidin-3-yloxymethyl)-phenylethynyl]-phenyl}-1-[3-(3-methyl-morpholin-4-yl)-propyl]-1,4,6,7-tetrahydro-pyrazolo[4,3-c]pyridin-5-yl}-2-oxo-acetamide

MS (ESI): mass calcd. for C₃₅H₄₀Cl₂N₆O₄, 678.3; m/z found, 679.2 [M+H]⁺. ¹H NMR (d₆-DMSO): 10.92 (br s, 1H), 8.91 (br s, 2H), 7.93 (s, 1H), 7.75-7.55 (m, 3H), 7.54-7.44 (m, 2H), 7.40-7.32 (m, 2H), 4.52 (s, 2H), 4.39 (s, 2H), 4.17 (s, 1H), 4.02-3.85 (m, 2H), 3.80-38 (m, 4H), 3.36-2.95 (m, 9H), 2.94-2.75 (m, 2H), 2.70-2.50 (m, 2H), 2.10-1.92 (m, 3H), 1.90-1.66 (m, 1H), 1.00 (d, J=8.2, 3H).

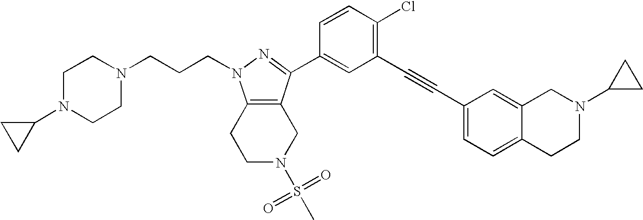

### Example 276

### 7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-2-cyclopropyl-1,2,3,4-tetrahydroisoquinoline

A. 7-Bromo-2-cyclopropyl-1,2,3,4-tetrahydro-isoquinoline. A sample of 7-bromo-1,2,3,4-tetrahydroisoquinoline hydrochloride (1.31 g, 5.27 mmol) was partitioned between 1.0 N NaOH (10 mL) and EtOAc (30 mL). The organic layer was concentrated. A solution of the resulting free amine in MeOH (7 mL), THF (7 mL) and acetic acid (1.5 mL) was treated with (1-ethoxy-cyclopropoxy)-trimethyl-silane (1.84 g, 10.5 mmol) followed by sodium cyanoborohydride (1.09 g. 17.4 mmol). The mixture was heated at 60° C. for 24 h. The mixture was partitioned between EtOAc and satd. aq. NaHCO₃. The aqueous layer was extracted with CH₂Cl₂. The combined organic layers were dried and concentrated. Purification of the residue (SiO₂; hexanes to 30% EtOAc/hexanes) afforded the desired product (1.17 g, 88%). ¹H NMR (CDCl₃): 7.22 (dd, J=8.2, 2.0, 1H), 7.18-7.15 (m, 1H), 6.94 (d, J=8.2, 1H), 3.74 (s, 2H), 2.90 (t, J=5.9, 2H), 2.80 (t, J=5.9, 2H), 1.82-1.74 (m, 4H).

B. The title compound was prepared according to the methods described for Example 134, substituting 7-bromo-2-cyclopropyl-1,2,3,4-tetrahydro-isoquinoline for 7-bromo-3,4-dihydro-1H-isoquinoline-2-carboxylic acid tert-butyl ester in Step B. MS (ESI): mass calcd. for C₃₇H₄₅ClN₆O₂S, 672.3; m/z found, 673.3 [M+H]⁺. ¹H NMR (CDCl₃): 7.70 (d, J=1.8, 1H), 7.42-7.36 (m, 2H), 7.29-7.26 (m, 1H), 7.22 (s, 1H), 7.02 (d, J=7.9, 1H), 4.47 (s, 2H), 4.03 (t, J=6.7, 2H), 3.73 (s, 2H), 3.59 (t, J=5.7, 2H), 2.90-2.80 (m, 9H), 2.70-2.26 (m, 10H), 2.11-1.99 (m, 2H), 1.78-1.71 (m, 1H), 1.62-1.55 (m, 1H), 0.50-0.44 (m, 4H), 0.41-0.32 (m, 4H).

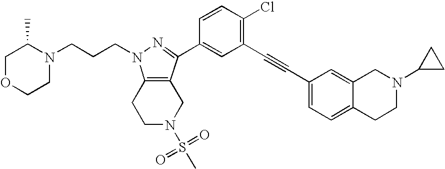

### Example 277

### 7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline

The title compound was prepared using methods analogous to those described for Example 276. MS (ESI): mass calcd. for C₃₅H₄₂ClN₅O₃S, 647.3; m/z found, 648.2 [M+H]⁺. ¹H NMR (CDCl₃): 7.77 (d, J=1.9, 1H), 7.50-7.43 (m, 2H), 7.36-7.33 (m, 1H), 7.30-7.29 (m, 1H), 7.09 (d, J=7.9, 1H), 4.54 (s, 2H), 4.15-4.00 (m, 2H), 3.83-3.76 (m, 3H), 3.71-3.59 (m, 4H), 3.28-3.21 (m, 1H), 2.98-2.70 (m, 12H), 2.47-2.36 (m, 1H), 2.32-2.19 (m, 2H), 2.16-1.98 (m, 1H), 1.85-1.78 (m, 1H), 0.92 (d, J=6.30, 3H), 0.57-0.51 (m, 4H).

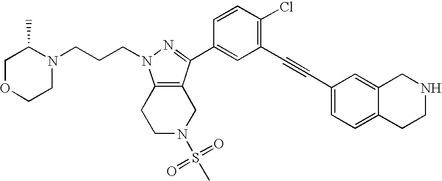

### Example 278

### 7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline

The title compound was prepared using methods analogous to those described for Example 134. MS (ESI): mass calcd. for C₃₂H₃₈ClN₅O₃S, 607.2; m/z found, 608.2 [M+H]⁺. ¹H NMR (CDCl₃): 7.78 (d, J=2.0, 1H), 7.53-7.43 (m, 2H), 7.39-7.35 (m, 1H), 7.32-7.27 (m, 1H), 7.10 (d, J=7.9, 1H), 4.54 (s, 2H), 4.14-4.00 (m, 3H), 3.82-3.75 (m, 1H), 3.70-3.59 (m, 4H), 3.32-3.14 (m, 3H), 2.97-2.70 (m, 10H), 2.46-2.35 (m, 1H), 2.32-2.18 (m, 2H), 2.16-1.97 (m, 2H), 1.77-1.68 (m, 1H), 0.92 (d, J=6.3, 3H).

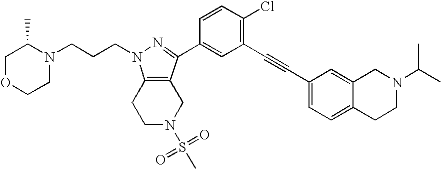

### Example 279

### 7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-(1-methylethyl)-1,2,3,4-tetrahydroisoquinoline

To a solution of 7-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline (0.05 g, 0.082 mmol) in CH₂Cl₂ (0.41 mL) was added acetone (7 mg, 0.121 mmol) followed by sodium triacetoxyborohydride (0.043 g, 0.206 mmol). After 18 h, the mixture was diluted with satd. aq. NaHCO₃ and extracted with CH₂Cl₂. The combined organic layers were dried and concentrated. Purification of the residue (SiO₂; CH₂Cl₂ to 3% MeOH/CH₂Cl₂) afforded the desired product (0.04 g, 75%). MS (ESI): mass calcd. for C₃₅H₄₄ClN₅O₃S, 649.3; m/z found, 650.2 [M+H]⁺. ¹H NMR (CDCl₃): 7.77 (d, J=1.9, 1H), 7.54-7.41 (m, 2H), 7.36-7.33 (m, 1H), 7.31-7.29 (m, 1H), 7.10 (d, J=7.9, 1H), 4.54 (s, 2H), 4.15-4.00 (m, 2H), 3.84-3.57 (m, 7H), 3.24 (dd, J=11.1, 8.7, 1H), 2.99-2.69 (m, 12H), 2.46-2.35 (m, 1H), 2.31-2.20 (m, 2H), 2.14-1.98 (m, 2H), 1.16 (d, J=6.5, 6H), 0.92 (d, J=6.3, 3H).

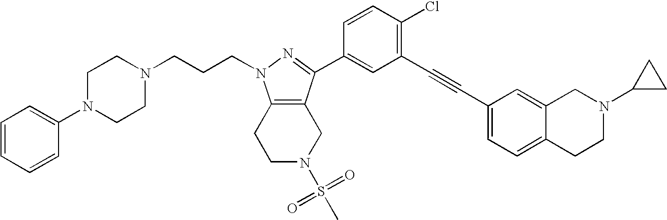

### Example 280

### 7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-cyclopropyl-1,2,3,4-tetrahydroisoquinoline

The title compound was prepared using methods analogous to those described for Example 276. MS (ESI): mass calcd. for C₄₀H₄₅ClN₆O₂S, 708.3; m/z found, 709.2 [M+H]⁺. ¹H NMR (CDCl₃): 7.79 (d, J=1.9, 1H), 7.53-7.43 (m, 2H), 7.37-7.33 (m, 1H), 7.31-7.23 (m, 3H), 7.09 (d, J=7.9, 1H), 6.95-6.91 (m, 2H), 6.89-6.84 (m, 1H), 4.54 (s, 2H), 4.12 (t, J=6.8, 2H), 3.80 (s, 2H), 3.65 (t, J=5.8, 2H), 3.22-3.17 (m, 4H), 2.97-2.87 (m, 9H), 2.60-2.56 (m, 4H), 2.38 (t, J=6.8, 2H), 2.17-2.08 (m, 2H), 1.86-1.77 (m, 1H), 0.58-0.50 (m, 4H).

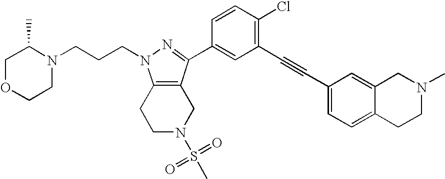

### Example 281

### 7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline

The title compound was prepared using methods analogous to those described for Example 279. MS (ESI): mass calcd. for C₃₃H₄₀ClN₅O₃S, 621.3; m/z found, 622.2 [M+H]⁺. ¹H NMR (CDCl₃): 7.78 (d, J=1.9, 1H), 7.51-7.42 (m, 2H), 7.38-7.35 (m, 1H), 7.28 (s, 1H), 7.11 (d, J=1.9, 1H), 4.54 (s, 2H), 4.14-4.00 (m, 2H), 3.82-3.76 (m, 1H), 3.72-3.58 (m, 6H), 3.24 (dd, J=11.2, 8.7, 1H), 2.98-2.69 (m, 11H), 2.48 (s, 3H), 2.45-2.36 (m, 1H), 2.31-2.19 (m, 2H), 2.15-1.97 (m, 2H), 0.92 (d, J=6.3, 3H).

The compounds in Examples 282-284 were prepared using methods analogous to those described for Example 276.

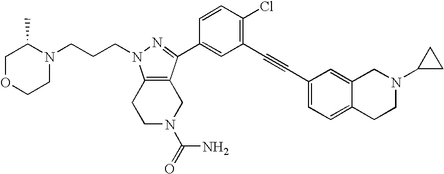

### Example 282

### 3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridine-5-carboxamide

MS (ESI): mass calcd. for C₃₅H₄₁ClN₆O₂, 612.3; m/z found, 613.2 [M+H]⁺. ¹H NMR (CDCl₃): 7.77 (d, J=2.1, 1H), 7.52 (dd, J=8.4, 2.1, 1H), 7.45 (d, J=8.4, 1H), 7.36-7.32 (m, 1H), 7.29-7.28 (m, 1H), 7.09 (d, J=8.4, 1H), 4.61-4.54 (m, 4H), 4.14-4.00 (m, 2H), 3.85-3.74 (m, 5H), 3.68-3.59 (m, 2H), 3.24 (dd, J=11.1, 8.8, 1H), 2.97-2.87 (m, 4H), 2.86-2.70 (m, 4H), 2.45-2.36 (m, 1H), 2.31-2.20 (m, 2H), 2.14-1.97 (m, 2H), 1.84-1.78 (m, 1H), 0.92 (d, J=6.3, 3H), 0.57-0.52 (m, 4H).

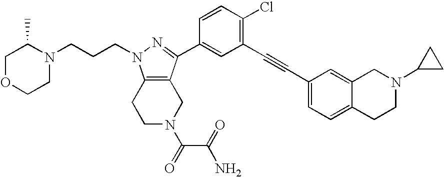

### Example 283

### 2-(3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide

MS (ESI): mass calcd. for C₃₆H₄₁ClN₆O₃, 640.3; m/z found, 641.2 [M+H]⁺. ¹H NMR (CDCl₃): 7.87-7.79 (m, 1H), 7.55-7.40 (m, 2H), 7.36-7.32 (m, 1H), 7.30-7.28 (m, 1H), 7.09 (d, J=7.9, 1H), 5.67-5.59 (m, 1H), 5.24-5.19 (m, 1H), 4.85-4.77 (m, 1H), 4.33-4.20 (m, 2H), 4.14-3.99 (m, 2H), 3.82-3.75 (m, 3H), 3.70-3.57 (m, 2H), 3.28-3.20 (m, 1H), 3.00-2.69 (m, 9H), 2.46-2.35 (m, 1H), 2.31-2.19 (m, 2H), 2.13-1.98 (m, 2H), 1.84-1.78 (m, 1H), 0.95-0.87 (m, 3H), 0.59-0.49 (m, 4H).

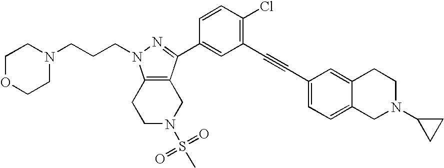

### Example 284

### 6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline

MS (ESI): mass calcd. for C₃₄H₄₀ClN₅O₃S, 633.3; m/z found, 634.2 [M+H]⁺. ¹H NMR (CDCl₃): 7.77 (d, J=1.8, 1H), 7.50-7.43 (m, 2H), 7.37-7.32 (m, 2H), 7.04 (d, J=8.4, 1H), 4.54 (s, 2H), 4.10 (t, J=6.8, 2H), 3.81 (s, 2H), 3.72-3.63 (m, 6H), 2.97-2.86 (m, 9H), 2.45-2.37 (m, 4H), 2.33 (t, J=6.9, 2H), 2.13-2.02 (m, 2H), 1.84-1.77 (m, 1H), 0.57-0.50 (m, 4H).

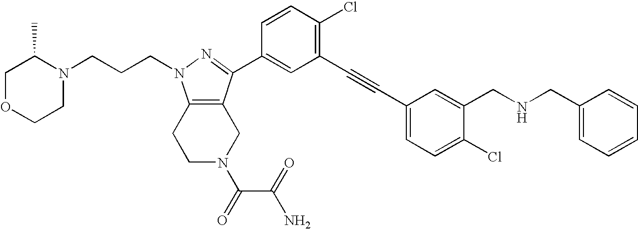

### Example 285

### 2-(3-{4-Chloro-3-[(4-chloro-3-{[(phenylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide

A. 2-Chloro-5-iodo-benzaldehyde. To a 0° C. solution of 2-chloro-5-iodo-benzonitrile (10.6 g, 40.4 mmol) in toluene (100 mL) was added DiBAL-H (1.5 M in toluene; 40.4 mL, 60.6 mmol). The mixture was stirred at 0° C. for 15 min and then warmed to rt for 3 h. The mixture was poured over 1.0 M H₂SO₄ (10 mL), MeOH (100 mL), and ice. The mixture was stirred vigorously and then was extracted with EtOAc (200 mL). The organic layer was dried and concentrated to give the title compound (2.98 g, 31%). ¹H NMR (CDCl₃): 10.37 (s, 1H), 8.22 (d, J=2.2, 1H), 7.83 (dd, J=8.4, 2.2, 1H), 7.21 (d, J=8.4, 1H).

B. Benzyl-(2-chloro-5-iodo-benzyl)-amine. To a slurry of 2-chloro-5-iodo-benzaldehyde (5.1 g, 21.5 mmol) and benzylamine (4.6 g, 43.1 mmol) in dichlorothethane (100 mL) was added sodium triacetoxyborohydride (13.7 g, 64.5 mmol). The reaction mixture was stirred for 18 h, then was diluted with satd. aq. NaHCO₃, and extracted with CH₂Cl₂ (2×). The combined organic extracts were washed with water (3×), dried, and concentrated. Purification by silica gel chromatography (hexanes to 20% EtOAc/hexanes) afforded the desired product (5.48 g, 80%).

C. To a solution of 2-[3-(4-chloro-3-iodo-phenyl)-1-(3-morpholin-4-yl-propyl)-1,4,6,7-tetrahydro-pyrazolo[4,3-c]pyridin-5-yl]-2-oxo-acetamide (prepared as described for Example 158, Step D; 103 mg, 0.180 mmol), PdCl₂(PPh)₃ (13 mg, 0.018 mmol), trimethylsilylacetylene (21 mg, 0.216 mmol), and CuI (3 mg, 0.018 mmol) in degassed DMF (1 mL) was added Et₃N (75 μL, 0.54 mmol). The reaction mixture was stirred under N₂ for 18 h. The mixture was then treated with benzyl-(2-chloro-5-iodo-benzyl)-amine (97 mg, 0.270 mmol), DBU (81 μL, 0.54 mmol), and degassed water (25 μL), and the reaction was stirred for an additional 24 h. The mixture was diluted with satd. aq. NaHCO₃ and extracted with CH₂Cl₂ (3×). The combined organic extracts were dried and concentrated. Purification by preparative thin layer chromatography (SiO₂; 7% MeOH/NH₃/CH₂Cl₂) afforded the desired product (68 mg, 54%). MS (ESI): mass calcd. for C₃₈H₄₀Cl₂N₆O₃, 698.3; m/z found, 699.2 [M+H]⁺. ¹H NMR (CDCl₃): 7.88-7.76 (m, 1H), 7.73-7.64 (m, 1H), 7.59-7.51 (m, 1H), 7.51-7.21 (m, 7H), 7.17-6.99 (m, 1H), 5.78-5.62 (m, 1H), 5.31-5.14 (m, 1H), 4.88-4.75 (m, 1H), 4.32-4.19 (m, 2H), 4.16-3.59 (m, 9H), 3.35-3.22 (m, 1H), 3.06-2.68 (m, 4H), 2.54-1.96 (m, 5H), 0.94 (d, J=5.8, 3H).

The compounds in Examples 286-287 were prepared using methods analogous to those described for Example 285.

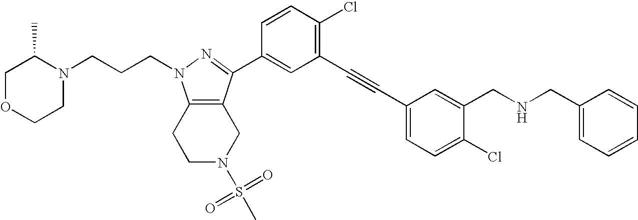

### Example 286

### 1-[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(phenylmethyl)methanamine

MS (ESI): mass calcd. for C₃₇H₄₁Cl₂N₅O₃S, 705.2; m/z found, 706.2 [M+H]⁺. ¹H NMR (CDCl₃): 7.79 (d, J=1.9, 1H), 7.67 (d, J=1.9, 1H), 7.53-7.41 (m, 3H), 7.40-7.32 (m, 5H), 7.30-7.24 (m, 1H), 4.61-4.46 (m, 2H), 4.17-3.99 (m, 2H), 3.92 (s, 2H), 3.85 (s, 2H), 3.83-3.76 (m, 1H), 3.73-3.59 (m, 4H), 3.25 (dd, J=11.1, 8.8, 1H), 2.98-2.83 (m, 5H), 2.83-2.69 (m, 2H), 2.48-2.36 (m, 1H), 2.33-2.20 (m, 2H), 2.17-1.96 (m, 2H), 0.92 (d, J=6.3, 3H).

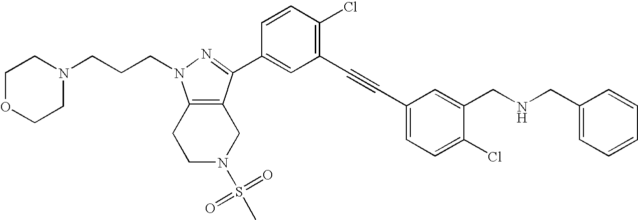

### Example 287

### 1-[2-Chloro-5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine

MS (ESI): mass calcd. for C₃₆H₃₉Cl₂N₅O₃S, 691.2; m/z found, 692.1 [M+H]⁺. ¹H NMR (CDCl₃): 7.79 (d, J=1.9, 1H), 7.67 (d, J=1.9, 1H), 7.53-7.41 (m, 3H), 7.41-7.31 (m, 5H), 7.29-7.24 (m, 1H), 4.54 (s, 2H), 4.11 (t, J=6.8, 2H), 3.92 (s, 2H), 3.85 (s, 2H), 3.74-3.63 (m, 6H), 2.93-2.87 (m, 5H), 2.47-2.37 (m, 4H), 2.33 (t, J=6.8, 2H), 2.13-2.03 (m, 2H).

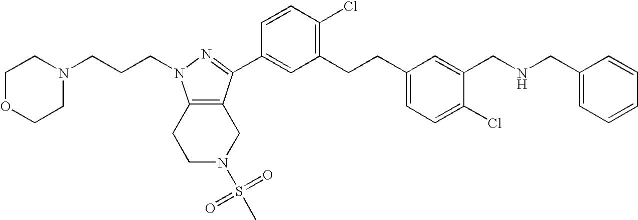

### Example 288

### 1-[2-Chloro-5-(2-{2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine

The title compound was prepared using methods analogous to those described for Example 2. MS (ESI): mass calcd. for C₃₆H₄₃Cl₂N₅O₃S, 695.3; m/z found, 696.2 [M+H]⁺. ¹H NMR (CDCl₃): 7.45 (d, J=2.0, 1H), 7.41-7.37 (m, 1H), 7.36-7.30 (m, 5H), 7.30-7.23 (m, 3H), 7.08 (dd, J=8.1, 2.2, 1H), 4.44 (s, 2H), 4.09 (t, J=6.8, 2H), 3.87 (s, 2H), 3.78 (s, 2H), 3.74-3.60 (m, 6H), 3.09-3.01 (m, 2H), 2.95-2.84 (m, 7H), 2.44-2.37 (m, 4H), 2.32 (t, J=6.8, 2H), 2.11-2.02 (m, 2H).

The compounds in Examples 289-295 were prepared using methods analogous to those described for Example 285.

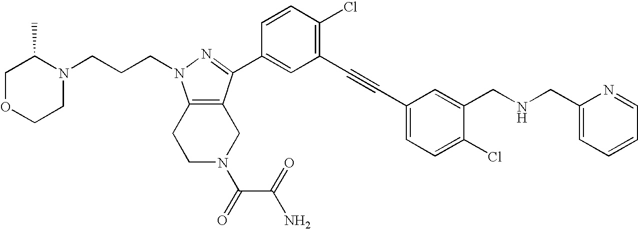

### Example 289

### 2-(3-{4-Chloro-3-[(4-chloro-3-{[(pyridin-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide

MS (ESI): mass calcd. for C₃₇H₃₉Cl₂N₇O₃, 699.3; m/z found, 700.2 [M+H]⁺. ¹H NMR (CDCl₃): 8.59-8.55 (m, 1H), 7.86-7.81 (m, 1H), 7.73-7.62 (m, 2H), 7.59-7.52 (m, 1H), 7.51-7.32 (m, 5H), 7.20-7.14 (m, 1H), 7.10-6.99 (m, 1H), 5.87-5.56 (m, 1H), 5.31-5.19 (m, 1H), 4.85-4.80 (m, 1H), 4.35-4.18 (m, 2H), 4.16-3.87 (m, 6H), 3.84-3.74 (m, 1H), 3.72-3.56 (m, 2H), 3.26 (t, J=9.9, 1H), 3.03-2.69 (m, 4H), 2.48-1.99 (m, 5H), 0.92 (d, J=6.2, 3H).

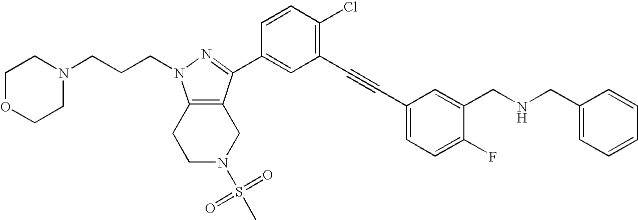

### Example 290

### 1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-(phenylmethyl)methanamine

MS (ESI): mass calcd. for C₃₆H₃₉ClFN₅O₃S, 675.2; m/z found, 676.2 [M+H]⁺. ¹H NMR (CDCl₃): 7.78 (d, J=1.9, 1H), 7.64 (dd, J=7.1, 2.1, 1H), 7.53-7.44 (m, 3H), 7.40-7.31 (m, 4H), 7.29-7.25 (m, 1H), 7.05 (dd, J=9.7, 8.5, 1H), 4.54 (s, 2H), 4.11 (t, J=6.8, 2H), 3.88 (s, 2H), 3.84 (s, 2H), 3.73-3.64 (m, 6H), 2.93-2.87 (m, 5H), 2.46-2.39 (m, 4H), 2.34 (t, J=6.8, 2H), 2.14-2.04 (m, 2H).

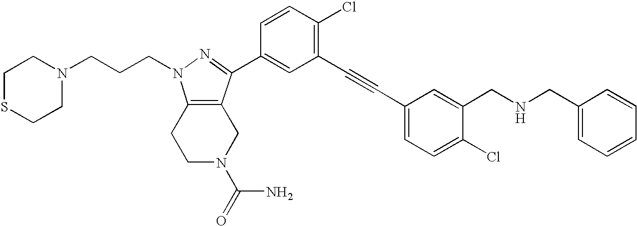

### Example 291

### 3-{4-Chloro-3-[(4-chloro-3-{[(phenylmethyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide

MS (ESI): mass calcd. for C₃₆H₃₈Cl₂N₆OS, 672.2; m/z found, 673.2 [M+H]⁺. ¹H NMR (CDCl₃): 7.80 (d, J=9.9, 1H), 7.66 (d, J=9.9, 1H), 7.56-7.51 (m, 1H), 7.48-7.31 (m, 7H), 7.30-7.25 (m, 1H), 4.68-4.51 (m, 4H), 4.07 (t, J=6.8, 2H), 3.92 (s, 2H), 3.85 (s, 2H), 3.77 (t, J=5.7, 2H), 2.79 (t, J=5.6, 2H), 2.71-2.62 (m, 8H), 2.35 (t, J=5.6, 2H), 2.10-2.01 (m, 2H).

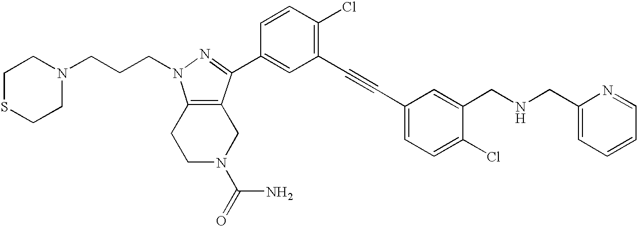

### Example 292

### 3-{4-Chloro-3-[(4-chloro-3-{[(pyridin-2-ylmethyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide

MS (ESI): mass calcd. for C₃₅H₃₇Cl₂N₇OS, 673.2; m/z found, 674.2 [M+H]⁺. ¹H NMR (CDCl₃): 8.63-8.51 (m, 1H), 7.78 (d, J=2.1, 1H), 7.71-7.63 (m, 2H), 7.57-7.52 (m, 1H), 7.49-7.31 (m, 4H), 7.21-7.15 (m, 1H), 4.66-4.52 (m, 4H), 4.08 (t, J=6.8, 2H), 3.98 (s, 2H), 3.97 (s, 2H), 3.78 (t, J=5.7, 2H), 2.79 (t, J=5.3, 2H), 2.71-2.62 (m, 8H), 2.36 (t, J=6.8, 2H), 2.10-2.00 (m, 2H).

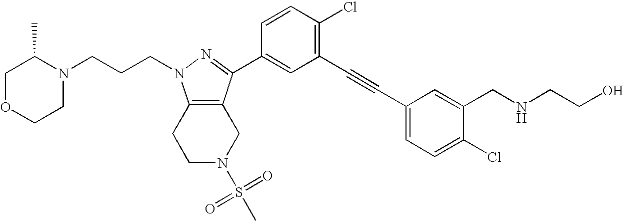

### Example 293

### 2-({[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]methyl}amino)ethanol

MS (ESI): mass calcd. for C₃₂H₃₉Cl₂N₅O₄S, 659.2; m/z found, 660.2 [M+H]⁺. ¹H NMR (CDCl₃): 7.79 (d, J=2.0, 1H), 7.62 (d, J=1.9, 1H), 7.54-7.42 (m, 3H), 7.37 (d, J=8.2, 1H), 4.55 (s, 2H), 4.16-4.00 (m, 2H), 3.93 (s, 2H), 3.82-3.76 (m, 1H), 3.73-3.58 (m, 6H), 3.25 (dd, J=11.2, 8.7, 1H), 2.96-2.70 (m, 9H), 2.47-2.37 (m, 1H), 2.33-2.19 (m, 2H), 2.15-1.98 (m, 2H), 0.92 (d, J=6.3, 3H).

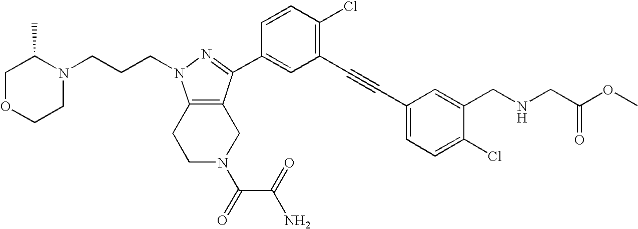

### Example 294

### Methyl N-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)methyl]glycinate

MS (ESI): mass calcd. for C₃₄H₃₈Cl₂N₆O₅, 680.2; m/z found, 681.3 [M+H]⁺. ¹H NMR (CDCl₃): 7.87-7.80 (m, 1H), 7.67-7.63 (m, 1H), 7.56 (dd, J=8.4, 2.2, 1H), 7.51-7.40 (m, 2H), 7.39-7.33 (m, 1H), 7.11-7.00 (m, 1H), 5.83-5.58 (m, 1H), 5.31-5.17 (m, 1H), 4.89-4.78 (m, 1H), 4.35-4.21 (m, 2H), 4.15-3.95 (m, 3H), 3.93 (s, 2H), 3.82-3.75 (m, 2H), 3.74 (s, 2H), 3.69-3.56 (m, 2H), 3.48 (s, 2H), 3.28-3.19 (m, 1H), 3.04-2.67 (m, 4H), 2.47-2.33 (m, 1H), 2.31-2.18 (m, 2H), 2.14-1.97 (m, 2H), 0.93-0.89 (m, 3H).

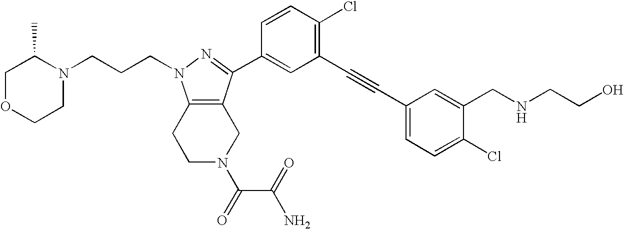

### Example 295

### 2-(3-{4-Chloro-3-[(4-chloro-3-{[(2-hydroxyethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide

MS (ESI): mass calcd. for C₃₃H₃₈Cl₂N₆O₄, 652.2; m/z found, 653.2 [M+H]⁺. ¹H NMR (CDCl₃): 7.83-7.74 (m, 1H), 7.69-7.64 (m, 1H), 7.61-7.39 (m, 4H), 5.17-5.10 (m, 1H), 4.81 (s, 1H), 4.20-3.92 (m, 6H), 3.87-3.59 (m, 5H), 3.41-3.35 (m, 2H), 3.30-3.24 (m, 1H), 3.01-2.72 (m, 6H), 2.52-2.40 (m, 1H), 2.38-2.24 (m, 2H), 2.15-1.96 (m, 2H), 0.96-0.93 (m, 3H).

The compounds in Table 1 (Examples 296-621) were prepared using methods analogous to those described in the preceding examples.

TABLE 1

| EX# | Chemical Name | MS Found |
|---|---|---|
| 296 | 3-(2-{3-[5-(Methylsulfonyl)-1-(3-morpholin-4-ylpropyl)-4,5,6,7-tetrahydro- | 525.60 |
| | 1H-pyrazolo[4,3-c]pyridin-3-yl]phenyl}ethyl)phenol | |
| 297 | 3-{4-Chloro-3-[(4-chlorophenyl)ethynyl]phenyl}-1-(3-morpholin-4-ylpropyl)- | 495.30 |
| | 4,5,6,7-tetrahydro-1H-pyrazolo[4,3-c]pyridine | |
| 298 | 8-{3-[3-{4-Chloro-3-[(4-chlorophenyl)ethynyl]phenyl}-5-(methylsulfonyl)- | 656.40 |
| | 4,5,6,7-tetrahydro-1H-pyrazolo[4,3-c]pyridin-1-yl]-2-hydroxypropyl}-2,8- | |
| | diazaspiro[4.5]decan-1-one | |
| 299 | 4-{2-Chloro-5-[5-(methylsulfonyl)-1-(3-morpholin-4-ylpropyl)-4,5,6,7- | 507.40 |
| | tetrahydro-1H-pyrazolo[4,3-c]pyridin-3-yl]phenyl}but-3-yn-1-ol | |
| 300 | 4-{2-Chloro-5-[5-(methylsulfonyl)-1-(3-morpholin-4-ylpropyl)-4,5,6,7- | 511.50 |
| | tetrahydro-1H-pyrazolo[4,3-c]pyridin-3-yl]phenyl}butan-1-ol | |
| 301 | 5-{2-Chloro-5-[5-(methylsulfonyl)-1-(3-morpholin-4-ylpropyl)-4,5,6,7- | 525.50 |
| | tetrahydro-1H-pyrazolo[4,3-c]pyridin-3-yl]phenyl}pentan-1-ol | |
| 302 | 3-{2-Chloro-5-[5-(methylsulfonyl)-1-(3-morpholin-4-ylpropyl)-4,5,6,7- | 497.50 |
| | tetrahydro-1H-pyrazolo[4,3-c]pyridin-3-yl]phenyl}propan-1-ol | |
| 303 | 3-{2-Chloro-5-[5-(methylsulfonyl)-1-(3-morpholin-4-ylpropyl)-4,5,6,7- | 492.40 |
| | tetrahydro-1H-pyrazolo[4,3-c]pyridin-3-yl]phenyl}prop-2-yn-1-amine | |
| 304 | 3-{2-Chloro-5-[5-(methylsulfonyl)-1-(3-morpholin-4-ylpropyl)-4,5,6,7- | 496.50 |
| | tetrahydro-1H-pyrazolo[4,3-c]pyridin-3-yl]phenyl}propan-1-amine | |
| 305 | N-(3-{5-[1-(2-Hydroxy-3-morpholin-4-ylpropyl)-5-(methylsulfonyl)-4,5,6,7- | 682.40 |
| | tetrahydro-1H-pyrazolo[4,3-c]pyridin-3-yl]-2-(trifluoromethyl)phenyl}prop-2- | |
| | yn-1-yl)benzenesulfonamide | |
| 306 | N-(3-{5-[1-(2-Hydroxy-3-morpholin-4-ylpropyl)-5-(methylsulfonyl)-4,5,6,7- | 686.50 |
| | tetrahydro-1H-pyrazolo[4,3-c]pyridin-3-yl]-2- | |
| | (trifluoromethyl)phenyl}propyl)benzenesulfonamide | |
| 307 | 1-[1-(3-{3-[3-(3-Aminoprop-1-yn-1-yl)-4-chlorophenyl]-5-(methylsulfonyl)- | 573.40 |
| | 4,5,6,7-tetrahydro-1H-pyrazolo[4,3-c]pyridin-1-yl}propyl)piperidin-4- | |
| | yl]pyrrolidin-2-one | |
| 308 | 1-[1-(3-{3-[3-(3-Aminopropyl)-4-chlorophenyl]-5-(methylsulfonyl)-4,5,6,7- | 577.30 |
| | tetrahydro-1H-pyrazolo[4,3-c]pyridin-1-yl}propyl)piperidin-4-yl]pyrrolidin-2- | |
| | one | |
| 309 | Methyl 2-{[(3-{2-chloro-5-[5-(methylsulfonyl)-1-{3-[4-(2-oxopyrrolidin-1- | 775.30 |
| | yl)piperidin-1-yl]propyl}-4,5,6,7-tetrahydro-1H-pyrazolo[4,3-c]pyridin-3- | |
| | yl]phenyl}propyl)amino]sulfonyl}benzoate | |
| 310 | 1-[1-(3-{3-[4-Chloro-3-(3-hydroxyprop-1-yn-1-yl)phenyl]-5-(methylsulfonyl)- | 574.30 |
| | 4,5,6,7-tetrahydro-1H-pyrazolo[4,3-c]pyridin-1-yl}propyl)piperidin-4- | |
| | yl]pyrrolidin-2-one | |
| 311 | 1-[1-(3-{3-[4-Chloro-3-(4-hydroxybutyl)phenyl]-5-(methylsulfonyl)-4,5,6,7- | 592.40 |
| | tetrahydro-1H-pyrazolo[4,3-c]pyridin-1-yl}propyl)piperidin-4-yl]pyrrolidin-2- | |
| | one | |
| 312 | 1-(1-{3-[3-{4-Chloro-3-[4-(dimethylamino)butyl]phenyl}-5-(methylsulfonyl)- | 619.40 |
| | 4,5,6,7-tetrahydro-1H-pyrazolo[4,3-c]pyridin-1-yl]propyl}piperidin-4- | |
| | yl)pyrrolidin-2-one | |
| 313 | 1-[1-(3-{3-[4-Chloro-3-(3-hydroxypropyl)phenyl]-5-(methylsulfonyl)-4,5,6,7- | 578.30 |
| | tetrahydro-1H-pyrazolo[4,3-c]pyridin-1-yl}propyl)piperidin-4-yl]pyrrolidin-2- | |
| | one | |
| 314 | 1-(1-{3-[3-{4-Chloro-3-[3-(dimethylamino)propyl]phenyl}-5-(methylsulfonyl)- | 605.40 |
| | 4,5,6,7-tetrahydro-1H-pyrazolo[4,3-c]pyridin-1-yl]propyl}piperidin-4- | |
| | yl)pyrrolidin-2-one | |
| 315 | 1-[4-({2-Chloro-5-[5-(methylsulfonyl)-1-(3-morpholin-4-ylpropyl)-4,5,6,7- | 582.30 |
| | tetrahydro-1H-pyrazolo[4,3-c]pyridin-3-yl]phenyl}ethynyl)phenyl]-N- | |
| | methylmethanamine | |
| 316 | N-{[4-({2-Chloro-5-[5-(methylsulfonyl)-1-(3-morpholin-4-ylpropyl)-4,5,6,7- | 672.40 |
| | tetrahydro-1H-pyrazolo[4,3-c]pyridin-3-yl]phenyl}ethynyl)phenyl]methyl}-2- | |
| | phenylethanamine | |
| 317 | N-{[4-({2-Chloro-5-[5-(methylsulfonyl)-1-(3-morpholin-4-ylpropyl)-4,5,6,7- | 624.40 |
| | tetrahydro-1H-pyrazolo[4,3-c]pyridin-3-yl]phenyl}ethynyl)phenyl]methyl}-N- | |
| | ethylethanamine | |
| 318 | N-{[4-({2-Chloro-5-[5-(methylsulfonyl)-1-(3-morpholin-4-ylpropyl)-4,5,6,7- | 624.40 |
| | tetrahydro-1H-pyrazolo[4,3-c]pyridin-3-yl]phenyl}ethynyl)phenyl]methyl}-2- | |
| | methylpropan-1-amine | |
| 319 | 1-[4-(2-{2-Chloro-5-[5-(methylsulfonyl)-1-(3-morpholin-4-ylpropyl)-4,5,6,7- | 696.30 |
| | tetrahydro-1H-pyrazolo[4,3-c]pyridin-3-yl]phenyl}ethyl)phenyl]-N-[(4- | |
| | chlorophenyl)methyl]methanamine | |
| 320 | 3-(3-{[4-(1H-Benzimidazol-2-yl)phenyl]ethynyl}-4-chlorophenyl)-5- | 655.30 |
| | (methylsulfonyl)-1-(3-morpholin-4-ylpropyl)-4,5,6,7-tetrahydro-1H- | |
| | pyrazolo[4,3-c]pyridine | |
| 321 | 4-({2-Chloro-5-[5-(methylsulfonyl)-1-(3-morpholin-4-ylpropyl)-4,5,6,7- | 644.40 |
| | tetrahydro-1H-pyrazolo[4,3-c]pyridin-3-yl]phenyl}ethynyl)-N- | |
| | (phenylmethyl)aniline | |
| 322 | {[4-({2-Chloro-5-[5-(methylsulfonyl)-1-(3-morpholin-4-ylpropyl)-4,5,6,7- | 593.30 |
| | tetrahydro-1H-pyrazolo[4,3-c]pyridin-3- | |
| | yl]phenyl}ethynyl)phenyl]amino}acetonitrile | |
| 323 | N-{[4-({2-Chloro-5-[5-(methylsulfonyl)-1-(3-morpholin-4-ylpropyl)-4,5,6,7- | 608.30 |
| | tetrahydro-1H-pyrazolo[4,3-c]pyridin-3- | |
| | yl]phenyl}ethynyl)phenyl]methyl}cyclopropanamine | |
| 324 | N-{[4-({2-Chloro-5-[5-(methylsulfonyl)-1-(3-morpholin-4-ylpropyl)-4,5,6,7- | 622.40 |
| | tetrahydro-1H-pyrazolo[4,3-c]pyridin-3- | |
| | yl]phenyl}ethynyl)phenyl]methyl}cyclobutanamine | |
| 325 | N-{[4-({2-Chloro-5-[5-(methylsulfonyl)-1-(3-morpholin-4-ylpropyl)-4,5,6,7- | 636.40 |
| | tetrahydro-1H-pyrazolo[4,3-c]pyridin-3- | |
| | yl]phenyl}ethynyl)phenyl]methyl}cyclopentanamine | |
| 326 | N-{[4-({2-Chloro-5-[5-(methylsulfonyl)-1-(3-morpholin-4-ylpropyl)-4,5,6,7- | 650.40 |
| | tetrahydro-1H-pyrazolo[4,3-c]pyridin-3- | |
| | yl]phenyl}ethynyl)phenyl]methyl}cyclohexanamine | |
| 327 | 4-({2-Chloro-5-[5-(methylsulfonyl)-1-(3-morpholin-4-ylpropyl)-4,5,6,7- | 658.30 |
| | tetrahydro-1H-pyrazolo[4,3-c]pyridin-3-yl]phenyl}ethynyl)-N-(2- | |
| | phenylethyl)aniline | |
| 328 | 1-(1-{3-[3-(4-Chloro-3-{[4-({[(4- | 773.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-yl)pyrrolidin-2-one | |
| 329 | 1,1-Dimethylethyl (1-{3-[3-(4-chloro-3-{[4-({[(4- | 805.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-yl)carbamate | |
| 330 | 1-{3-[3-(4-Chloro-3-{[4-({[(4- | 706.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-ol | |
| 331 | 1-{3-[3-(4-Chloro-3-{[4-({[(4- | 705.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-amine | |
| 332 | 1-[3-(4-Chloro-3-{[4-({[(4- | 708.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]-3- | |
| | morpholin-4-ylpropan-2-ol | |
| 333 | 1-[4-({2-Chloro-5-[5-(methylsulfonyl)-1-(3-pyrrolidin-1-ylpropyl)-4,5,6,7- | 676.20 |
| | tetrahydro-1H-pyrazolo[4,3-c]pyridin-3-yl]phenyl}ethynyl)phenyl]-N-[(4- | |
| | chlorophenyl)methyl]methanamine | |
| 334 | Ethyl 1-{3-[3-(4-chloro-3-{[4-({[(4- | 762.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidine-4-carboxylate | |
| 335 | 1-{4-[(2-Chloro-5-{1-[3-(1,4-dioxa-8-azaspiro[4.5]dec-8-yl)propyl]-5- | 748.30 |
| | (methylsulfonyl)-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 336 | 1-{3-[3-(4-Chloro-3-{[4-({[(4- | 734.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidine-4-carboxylic acid | |
| 337 | (1-{3-[3-(4-Chloro-3-{[4-({[(4- | 720.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-yl)methanol | |
| 338 | 1′-{3-[3-(4-Chloro-3-{[4-({[(4- | 787.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]propyl}- | |
| | 1,4′-bipiperidin-2-one | |
| 339 | 1-[4-({2-Chloro-5-[5-(methylsulfonyl)-1-(3-morpholin-4-ylpropyl)-4,5,6,7- | 688.30 |
| | tetrahydro-1H-pyrazolo[4,3-c]pyridin-3-yl]phenyl}ethynyl)phenyl]-N-{[4- | |
| | (methyloxy)phenyl]methyl}methanamine | |
| 340 | N-{[4-({2-Chloro-5-[5-(methylsulfonyl)-1-(3-morpholin-4-ylpropyl)-4,5,6,7- | 650.20 |
| | tetrahydro-1H-pyrazolo[4,3-c]pyridin-3-yl]phenyl}ethynyl)phenyl]methyl}- | |
| | 2,2,2-trifluoroethanamine | |
| 341 | 1-[4-({2-Chloro-5-[5-(methylsulfonyl)-1-(3-morpholin-4-ylpropyl)-4,5,6,7- | 622.30 |
| | tetrahydro-1H-pyrazolo[4,3-c]pyridin-3-yl]phenyl}ethynyl)phenyl]-N- | |
| | (cyclopropylmethyl)methanamine | |
| 342 | (2S)-2-({[4-({2-Chloro-5-[5-(methylsulfonyl)-1-(3-morpholin-4-ylpropyl)- | 688.30 |
| | 4,5,6,7-tetrahydro-1H-pyrazolo[4,3-c]pyridin-3- | |
| | yl]phenyl}ethynyl)phenyl]methyl}amino)-2-phenylethanol | |
| 343 | 1-{4-[(2-Chloro-5-{5-(methylsulfonyl)-1-[3-(4-morpholin-4-ylpiperidin-1- | 775.30 |
| | yl)propyl]-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 344 | 1-{4-[(2-Chloro-5-{1-[3-(4-methylpiperidin-1-yl)propyl]-5-(methylsulfonyl)- | 704.30 |
| | 4,5,6,7-tetrahydro-1H-pyrazolo[4,3-c]pyridin-3-yl}phenyl)ethynyl]phenyl}-N- | |
| | [(4-chlorophenyl)methyl]methanamine | |
| 345 | 1-[4-({2-Chloro-5-[5-(methylsulfonyl)-1-{3-[4-(trifluoromethyl)piperidin-1- | 758.30 |
| | yl]propyl}-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 346 | N-(1-{3-[3-(4-Chloro-3-{[4-({[(4- | 747.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-yl)acetamide | |
| 347 | Methyl N-{[4-({2-chloro-5-[5-(methylsulfonyl)-1-(3-morpholin-4-ylpropyl)- | 640.30 |
| | 4,5,6,7-tetrahydro-1H-pyrazolo[4,3-c]pyridin-3- | |
| | yl]phenyl}ethynyl)phenyl]methyl}glycinate | |
| 348 | 1-(1-{3-[3-{4-Chloro-3-[(4-{[(2,2,2- | 731.30 |
| | trifluoroethyl)amino]methyl}phenyl)ethynyl]phenyl}-5-(methylsulfonyl)- | |
| | 4,5,6,7-tetrahydro-1H-pyrazolo[4,3-c]pyridin-1-yl]propyl}piperidin-4- | |
| | yl)pyrrolidin-2-one | |
| 349 | N-{[4-({2-Chloro-5-[5-(methylsulfonyl)-1-(3-morpholin-4-ylpropyl)-4,5,6,7- | 608.30 |
| | tetrahydro-1H-pyrazolo[4,3-c]pyridin-3- | |
| | yl]phenyl}ethynyl)phenyl]methyl}prop-2-en-1-amine | |
| 350 | 1-{3-[3-(4-Chloro-3-{[4-({[(4- | 733.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidine-4-carboxamide | |
| 351 | Methyl (2S)-({[4-({2-chloro-5-[5-(methylsulfonyl)-1-(3-morpholin-4-ylpropyl)- | 716.30 |
| | 4,5,6,7-tetrahydro-1H-pyrazolo[4,3-c]pyridin-3- | |
| | yl]phenyl}ethynyl)phenyl]methyl}amino)(phenyl)ethanoate | |
| 352 | 1-(1-{3-[3-(4-Chloro-3-{[4-({[(1R)-2-hydroxy-1- | 769.40 |
| | phenylethyl]amino}methyl)phenyl]ethynyl}phenyl)-5-(methylsulfonyl)- | |
| | 4,5,6,7-tetrahydro-1H-pyrazolo[4,3-c]pyridin-1-yl]propyl}piperidin-4- | |
| | yl)pyrrolidin-2-one | |
| 353 | 1-{4-[(5-{1-[3-(4-Acetylpiperazin-1-yl)propyl]-5-(methylsulfonyl)-4,5,6,7- | 733.40 |
| | tetrahydro-1H-pyrazolo[4,3-c]pyridin-3-yl}-2-chlorophenyl)ethynyl]phenyl}- | |
| | N-[(4-chlorophenyl)methyl]methanamine | |
| 354 | 1-{4-[(2-Chloro-5-{1-[3-(4-methylpiperazin-1-yl)propyl]-5-(methylsulfonyl)- | 705.30 |
| | 4,5,6,7-tetrahydro-1H-pyrazolo[4,3-c]pyridin-3-yl}phenyl)ethynyl]phenyl}-N- | |
| | [(4-chlorophenyl)methyl]methanamine | |
| 355 | 1-{4-[(2-Chloro-5-{1-[3-(4,4-dimethylpiperidin-1-yl)propyl]-5- | 718.30 |
| | (methylsulfonyl)-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 356 | N-(1-{3-[3-(4-Chloro-3-{[4-({[(4- | 763.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-yl)-2-hydroxyacetamide | |
| 357 | 1-{4-[(2-Chloro-5-{1-[3-(4,4-difluoropiperidin-1-yl)propyl]-5-(methylsulfonyl)- | 726.30 |
| | 4,5,6,7-tetrahydro-1H-pyrazolo[4,3-c]pyridin-3-yl}phenyl)ethynyl]phenyl}-N- | |
| | [(4-chlorophenyl)methyl]methanamine | |
| 358 | 1-{4-[(2-Chloro-5-{1-[3-(4-fluoropiperidin-1-yl)propyl]-5-(methylsulfonyl)- | 708.10 |
| | 4,5,6,7-tetrahydro-1H-pyrazolo[4,3-c]pyridin-3-yl}phenyl)ethynyl]phenyl}-N- | |
| | [(4-chlorophenyl)methyl]methanamine | |
| 359 | N-(1-{3-[3-(4-Chloro-3-{[4-({[(4- | 783.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperidin-4-yl)methanesulfonamide | |
| 360 | 7-({2-Chloro-5-[5-(methylsulfonyl)-1-(3-morpholin-4-ylpropyl)-4,5,6,7- | 594.20 |
| | tetrahydro-1H-pyrazolo[4,3-c]pyridin-3-yl]phenyl}ethynyl)-1,2,3,4- | |
| | tetrahydroisoquinoline | |
| 361 | 1-[3-(4-Chloro-3-{[4-({[(4- | 706.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]-3- | |
| | piperidin-1-ylpropan-2-ol | |
| 362 | N-(1-{3-[3-(4-Chloro-3-{[4-({[(4- | 763.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]-2- | |
| | hydroxypropyl}piperidin-4-yl)acetamide | |
| 363 | 1-{3-[3-(4-Chloro-3-{[4-({[(4- | 749.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]-2- | |
| | hydroxypropyl}piperidine-4-carboxamide | |
| 364 | 3-(4-Chloro-3-{[2-(trifluoroacetyl)-2,3-dihydro-1H-isoindol-5- | 676.20 |
| | yl]ethynyl}phenyl)-5-(methylsulfonyl)-1-(3-morpholin-4-ylpropyl)-4,5,6,7- | |
| | tetrahydro-1H-pyrazolo[4,3-c]pyridine | |
| 365 | 6-({2-Chloro-5-[5-(methylsulfonyl)-1-(3-morpholin-4-ylpropyl)-4,5,6,7- | 594.20 |
| | tetrahydro-1H-pyrazolo[4,3-c]pyridin-3-yl]phenyl}ethynyl)-1,2,3,4- | |
| | tetrahydroisoquinoline | |
| 366 | 8-({2-Chloro-5-[5-(methylsulfonyl)-1-(3-morpholin-4-ylpropyl)-4,5,6,7- | 594.20 |
| | tetrahydro-1H-pyrazolo[4,3-c]pyridin-3-yl]phenyl}ethynyl)-1,2,3,4- | |
| | tetrahydroisoquinoline | |
| 367 | 1,1-Dimethylethyl 4-{3-[3-(4-chloro-3-{[4-({[(4- | 791.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperazine-1-carboxylate | |
| 368 | 1-[4-({2-Chloro-5-[5-(methylsulfonyl)-1-(3-piperazin-1-ylpropyl)-4,5,6,7- | 691.30 |
| | tetrahydro-1H-pyrazolo[4,3-c]pyridin-3-yl]phenyl}ethynyl)phenyl]-N-[(4- | |
| | chlorophenyl)methyl]methanamine | |
| 369 | N-(1-{3-[3-(4-Chloro-3-{[4-({[(4- | 669.30 |
| | chlorophenyl)methyl]amino}methyl)phenyl]ethynyl}phenyl)-4,5,6,7- | |
| | tetrahydro-1H-pyrazolo[4,3-c]pyridin-1-yl]propyl}piperidin-4-yl)acetamide | |
| 370 | 1,1-Dimethylethyl 7-({2-chloro-5-[5-(methylsulfonyl)-1-(3-morpholin-4- | 694.30 |
| | ylpropyl)-4,5,6,7-tetrahydro-1H-pyrazolo[4,3-c]pyridin-3-yl]phenyl}ethynyl)- | |
| | 3,4-dihydroisoquinoline-2(1H)-carboxylate | |
| 371 | 1,1-Dimethylethyl 7-({2-chloro-5-[1-(2-hydroxy-3-piperidin-1-ylpropyl)-5- | 708.30 |
| | (methylsulfonyl)-4,5,6,7-tetrahydro-1H-pyrazolo[4,3-c]pyridin-3- | |
| | yl]phenyl}ethynyl)-3,4-dihydroisoquinoline-2(1H)-carboxylate | |
| 372 | 1-[4-({2-Chloro-5-[1-{3-[4-(1,1-dimethylethyl)piperidin-1-yl]propyl}-5- | 746.30 |
| | (methylsulfonyl)-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 373 | 1,1-Dimethylethyl 7-({5-[1-{3-[4-(aminocarbonyl)piperidin-1-yl]-2- | 751.30 |
| | hydroxypropyl}-5-(methylsulfonyl)-4,5,6,7-tetrahydro-1H-pyrazolo[4,3- | |
| | c]pyridin-3-yl]-2-chlorophenyl}ethynyl)-3,4-dihydroisoquinoline-2(1H)- | |
| | carboxylate | |
| 374 | 1,1-Dimethylethyl 7-({5-[1-{3-[4-(aminocarbonyl)piperidin-1-yl]propyl}-5- | 735.40 |
| | (methylsulfonyl)-4,5,6,7-tetrahydro-1H-pyrazolo[4,3-c]pyridin-3-yl]-2- | |
| | chlorophenyl}ethynyl)-3,4-dihydroisoquinoline-2(1H)-carboxylate | |
| 375 | 7-({2-Chloro-5-[5-(methylsulfonyl)-1-(3-morpholin-4-ylpropyl)-4,5,6,7- | 608.30 |
| | tetrahydro-1H-pyrazolo[4,3-c]pyridin-3-yl]phenyl}ethynyl)-2,3,4,5- | |
| | tetrahydro-1H-3-benzazepine | |
| 376 | 1,1-Dimethylethyl {[3-({2-chloro-5-[5-(methylsulfonyl)-1-(3-morpholin-4- | 668.30 |
| | ylpropyl)-4,5,6,7-tetrahydro-1H-pyrazolo[4,3-c]pyridin-3- | |
| | yl]phenyl}ethynyl)phenyl]methyl}carbamate | |
| 377 | 1-[3-({2-Chloro-5-[5-(methylsulfonyl)-1-(3-morpholin-4-ylpropyl)-4,5,6,7- | 568.20 |
| | tetrahydro-1H-pyrazolo[4,3-c]pyridin-3- | |
| | yl]phenyl}ethynyl)phenyl]methanamine | |
| 378 | 7-({2-Chloro-5-[1-{3-[4-(1,1-dimethylethyl)piperidin-1-yl]propyl}-5- | 648.30 |
| | (methylsulfonyl)-4,5,6,7-tetrahydro-1H-pyrazolo[4,3-c]pyridin-3- | |
| | yl]phenyl}ethynyl)-1,2,3,4-tetrahydroisoquinoline | |
| 379 | 1-[3-({2-Chloro-5-[5-(methylsulfonyl)-1-(3-morpholin-4-ylpropyl)-4,5,6,7- | 658.20 |
| | tetrahydro-1H-pyrazolo[4,3-c]pyridin-3-yl]phenyl}ethynyl)phenyl]-N- | |
| | (phenylmethyl)methanamine | |
| 380 | 1-[4-({2-Chloro-5-[5-(methylsulfonyl)-1-{3-[4-(phenylcarbonyl)piperazin-1- | 795.30 |
| | yl]propyl}-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 381 | 7-({2-Chloro-5-[5-(methylsulfonyl)-1-(3-piperidin-1-ylpropyl)-4,5,6,7- | |
| | tetrahydro-1H-pyrazolo[4,3-c]pyridin-3-yl]phenyl}ethynyl)-1,2,3,4- | |
| | tetrahydroisoquinoline | |
| 382 | (3S)-1-{3-[3-(4-Chloro-3-{[4-({[(4- | 692.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}pyrrolidin-3-ol | |
| 383 | (3R)-1-{3-[3-(4-Chloro-3-{[4-({[(4- | 692.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}pyrrolidin-3-ol | |
| 384 | 1,1-Dimethylethyl {[2-({2-chloro-5-[5-(methylsulfonyl)-1-(3-morpholin-4- | 668.30 |
| | ylpropyl)-4,5,6,7-tetrahydro-1H-pyrazolo[4,3-c]pyridin-3- | |
| | yl]phenyl}ethynyl)phenyl]methyl}carbamate | |
| 385 | 1-[2-({2-Chloro-5-[5-(methylsulfonyl)-1-(3-morpholin-4-ylpropyl)-4,5,6,7- | 568.20 |
| | tetrahydro-1H-pyrazolo[4,3-c]pyridin-3- | |
| | yl]phenyl}ethynyl)phenyl]methanamine | |
| 386 | 1-[2-({2-Chloro-5-[5-(methylsulfonyl)-1-(3-morpholin-4-ylpropyl)-4,5,6,7- | 658.30 |
| | tetrahydro-1H-pyrazolo[4,3-c]pyridin-3-yl]phenyl}ethynyl)phenyl]-N- | |
| | (phenylmethyl)methanamine | |
| 387 | 1-[4-({2-Chloro-5-[1-{3-[(2R,6S)-2,6-dimethylmorpholin-4-yl]propyl}-5- | 720.30 |
| | (methylsulfonyl)-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 388 | 1-{4-[(2-Chloro-5-{1-[3-(4-cyclopropylpiperazin-1-yl)propyl]-5- | 731.30 |
| | (methylsulfonyl)-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 389 | (4-{3-[3-(4-Chloro-3-{[4-({[(4- | 722.30 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}morpholin-2-yl)methanol | |
| 390 | 1,1-Dimethylethyl 4-{3-[3-(4-chloro-3-{[4-({[(4- | 805.40 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]propyl}- | |
| | 1,4-diazepane-1-carboxylate | |
| 391 | 1-{4-[(2-Chloro-5-{1-[3-(1,4-diazepan-1-yl)propyl]-5-(methylsulfonyl)- | 705.20 |
| | 4,5,6,7-tetrahydro-1H-pyrazolo[4,3-c]pyridin-3-yl}phenyl)ethynyl]phenyl}-N- | |
| | [(4-chlorophenyl)methyl]methanamine | |
| 392 | 1,1-Dimethylethyl 5-({2-chloro-5-[5-(methylsulfonyl)-1-(3-morpholin-4- | 680.10 |
| | ylpropyl)-4,5,6,7-tetrahydro-1H-pyrazolo[4,3-c]pyridin-3-yl]phenyl}ethynyl)- | |
| | 1,3-dihydro-2H-isoindole-2-carboxylate | |
| 393 | 3-[4-Chloro-3-(2,3-dihydro-1H-isoindol-5-ylethynyl)phenyl]-5- | 580.10 |
| | (methylsulfonyl)-1-(3-morpholin-4-ylpropyl)-4,5,6,7-tetrahydro-1H- | |
| | pyrazolo[4,3-c]pyridine | |
| 394 | 3-({2-Chloro-5-[5-(methylsulfonyl)-1-(3-morpholin-4-ylpropyl)-4,5,6,7- | 595.10 |
| | tetrahydro-1H-pyrazolo[4,3-c]pyridin-3-yl]phenyl}ethynyl)-5,6,7,8- | |
| | tetrahydro-1,6-naphthyridine | |
| 395 | 1-{4-[2-(2-Chloro-5-{5-(methylsulfonyl)-1-[3-(4-phenylpiperazin-1-yl)propyl]- | 771.10 |
| | 4,5,6,7-tetrahydro-1H-pyrazolo[4,3-c]pyridin-3-yl}phenyl)ethyl]phenyl}-N-[(4- | |
| | chlorophenyl)methyl]methanamine | |
| 396 | 7-({2-Chloro-5-[5-(methylsulfonyl)-1-(3-morpholin-4-ylpropyl)-4,5,6,7- | 634.50 |
| | tetrahydro-1H-pyrazolo[4,3-c]pyridin-3-yl]phenyl}ethynyl)-2-cyclopropyl- | |
| | 1,2,3,4-tetrahydroisoquinoline | |
| 397 | 4-{3-[3-(4-Chloro-3-{[4-({[(4- | 705.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperazin-2-one | |
| 398 | 1-{4-[(2-Chloro-5-{1-[3-(1,1-dioxidothiomorpholin-4-yl)propyl]-5- | 740.00 |
| | (methylsulfonyl)-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 399 | 1-{4-[(2-Chloro-5-{5-(methylsulfonyl)-1-[3-(1,4-oxazepan-4-yl)propyl]- | 706.10 |
| | 4,5,6,7-tetrahydro-1H-pyrazolo[4,3-c]pyridin-3-yl}phenyl)ethynyl]phenyl}-N- | |
| | [(4-chlorophenyl)methyl]methanamine | |
| 400 | 1-(4-{[5-(5-Acetyl-1-{3-[(3S)-3-methylmorpholin-4-yl]propyl}-4,5,6,7- | 670.20 |
| | tetrahydro-1H-pyrazolo[4,3-c]pyridin-3-yl)-2-chlorophenyl]ethynyl}phenyl)- | |
| | N-[(4-chlorophenyl)methyl]methanamine | |
| 401 | 3-(4-Chloro-3-{[4-({[(4- | 671.20 |
| | chlorophenyl)methyl]amino}methyl)phenyl]ethynyl}phenyl)-1-{3-[(3S)-3- | |
| | methylmorpholin-4-yl]propyl}-1,4,6,7-tetrahydro-5H-pyrazolo[4,3-c]pyridine- | |
| | 5-carboxamide | |
| 402 | 2-[3-(4-Chloro-3-{[4-({[(4- | 686.20 |
| | chlorophenyl)methyl]amino}methyl)phenyl]ethynyl}phenyl)-1-{3-[(3S)-3- | |
| | methylmorpholin-4-yl]propyl}-1,4,6,7-tetrahydro-5H-pyrazolo[4,3-c]pyridin- | |
| | 5-yl]-2-oxoethanol | |
| 403 | 3-({2-Chloro-5-[5-(methylsulfonyl)-1-(3-morpholin-4-ylpropyl)-4,5,6,7- | 585.10 |
| | tetrahydro-1H-pyrazolo[4,3-c]pyridin-3-yl]phenyl}ethynyl)-5,6,7,8- | |
| | tetrahydro[1,2,4]triazolo[4,3-a]pyrazine | |
| 404 | 1-[4-({2-Chloro-5-[5-(methylsulfonyl)-1-(3-morpholin-4-ylpropyl)-4,5,6,7- | 659.10 |
| | tetrahydro-1H-pyrazolo[4,3-c]pyridin-3-yl]phenyl}ethynyl)phenyl]-N-(pyridin- | |
| | 3-ylmethyl)methanamine | |
| 405 | 1-[4-({2-Chloro-5-[5-(methylsulfonyl)-1-(3-morpholin-4-ylpropyl)-4,5,6,7- | 659.10 |
| | tetrahydro-1H-pyrazolo[4,3-c]pyridin-3-yl]phenyl}ethynyl)phenyl]-N-(pyridin- | |
| | 4-ylmethyl)methanamine | |
| 406 | 4-{3-[3-(4-Chloro-3-{[4-({[(4- | 719.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]propyl}-3- | |
| | methylpiperazin-2-one | |
| 407 | 2-(4-{3-[3-(4-Chloro-3-{[4-({[(4- | 783.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperazin-1-yl)phenol | |
| 408 | 1-[4-({2-Chloro-5-[5-(methylsulfonyl)-1-(3-morpholin-4-ylpropyl)-4,5,6,7- | 659.10 |
| | tetrahydro-1H-pyrazolo[4,3-c]pyridin-3-yl]phenyl}ethynyl)phenyl]-N-(pyridin- | |
| | 2-ylmethyl)methanamine | |
| 409 | 3-(4-{3-[3-(4-Chloro-3-{[4-({[(4- | 783.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperazin-1-yl)phenol | |
| 410 | 4-(4-{3-[3-(4-Chloro-3-{[4-({[(4- | 783.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}piperazin-1-yl)phenol | |
| 411 | 3-[3-(1H-Benzimidazol-5-ylethynyl)-4-chlorophenyl]-5-(methylsulfonyl)-1-(3- | 579.00 |
| | morpholin-4-ylpropyl)-4,5,6,7-tetrahydro-1H-pyrazolo[4,3-c]pyridine | |
| 412 | 1-[4-({5-[5-Acetyl-1-(3-thiomorpholin-4-ylpropyl)-4,5,6,7-tetrahydro-1H- | 672.22 |
| | pyrazolo[4,3-c]pyridin-3-yl]-2-chlorophenyl}ethynyl)phenyl]-N-[(4- | |
| | chlorophenyl)methyl]methanamine | |
| 413 | 2-[3-(4-Chloro-3-{[4-({[(4- | 690.20 |
| | chlorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridin-5-yl]- | |
| | 2-oxoethanol | |
| 414 | 2-[3-(4-Chloro-3-{[4-({[(4- | 703.20 |
| | chlorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridin-5-yl]- | |
| | 2-oxoacetamide | |
| 415 | 7-[(2-Chloro-5-{1-[3-(4-cyclopropylpiperazin-1-yl)propyl]-5-(methylsulfonyl)- | 633.20 |
| | 4,5,6,7-tetrahydro-1H-pyrazolo[4,3-c]pyridin-3-yl}phenyl)ethynyl]-1,2,3,4- | |
| | tetrahydroisoquinoline | |
| 416 | 1-{4-[(2-Chloro-5-{5-(methylsulfonyl)-1-[3-(4-pyridin-4-ylpiperazin-1- | 768.10 |
| | yl)propyl]-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 417 | 1-{4-[(2-Chloro-5-{5-(methylsulfonyl)-1-[3-(4-pyridin-3-ylpiperazin-1- | 768.10 |
| | yl)propyl]-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 418 | Methyl 4-{3-[3-(4-chloro-3-{[4-({[(4- | 750.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}morpholine-3-carboxylate | |
| 419 | (4-{3-[3-(4-Chloro-3-{[4-({[(4- | 722.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}morpholin-3-yl)methanol | |
| 420 | 1-[4-({2-Chloro-5-[5-(methylsulfonyl)-1-{3-[(1S,4S)-2-oxa-5- | 704.10 |
| | azabicyclo[2.2.1]hept-5-yl]propyl}-4,5,6,7-tetrahydro-1H-pyrazolo[4,3- | |
| | c]pyridin-3-yl]phenyl}ethynyl)phenyl]-N-[(4- | |
| | chlorophenyl)methyl]methanamine | |
| 421 | 1-[4-({2-Chloro-5-[5-(methylsulfonyl)-1-(3-morpholin-4-ylpropyl)-4,5,6,7- | 664.00 |
| | tetrahydro-1H-pyrazolo[4,3-c]pyridin-3-yl]phenyl}ethynyl)phenyl]-N-(2- | |
| | thienylmethyl)methanamine | |
| 422 | 1-[4-({2-Chloro-5-[5-(methylsulfonyl)-1-(3-morpholin-4-ylpropyl)-4,5,6,7- | 664.10 |
| | tetrahydro-1H-pyrazolo[4,3-c]pyridin-3-yl]phenyl}ethynyl)phenyl]-N-(3- | |
| | thienylmethyl)methanamine | |
| 423 | N-{[4-({2-Chloro-5-[5-(methylsulfonyl)-1-(3-morpholin-4-ylpropyl)-4,5,6,7- | 678.10 |
| | tetrahydro-1H-pyrazolo[4,3-c]pyridin-3-yl]phenyl}ethynyl)phenyl]methyl}-2- | |
| | (2-thienyl)ethanamine | |
| 424 | 1-[4-({2-Chloro-5-[5-(methylsulfonyl)-1-(3-morpholin-4-ylpropyl)-4,5,6,7- | 678.10 |
| | tetrahydro-1H-pyrazolo[4,3-c]pyridin-3-yl]phenyl}ethynyl)phenyl]-N-[(3- | |
| | methyl-2-thienyl)methyl]methanamine | |
| 425 | 1-[4-({2-Chloro-5-[5-(methylsulfonyl)-1-(3-morpholin-4-ylpropyl)-4,5,6,7- | 648.10 |
| | tetrahydro-1H-pyrazolo[4,3-c]pyridin-3-yl]phenyl}ethynyl)phenyl]-N-(furan-2- | |
| | ylmethyl)methanamine | |
| 426 | 1-[4-({2-Chloro-5-[5-(methylsulfonyl)-1-(3-morpholin-4-ylpropyl)-4,5,6,7- | 730.10 |
| | tetrahydro-1H-pyrazolo[4,3-c]pyridin-3-yl]phenyl}ethynyl)phenyl]-N-{[5- | |
| | methyl-2-(trifluoromethyl)furan-3-yl]methyl}methanamine | |
| 427 | 1-[5-({2-Chloro-5-[5-(methylsulfonyl)-1-(3-morpholin-4-ylpropyl)-4,5,6,7- | 659.10 |
| | tetrahydro-1H-pyrazolo[4,3-c]pyridin-3-yl]phenyl}ethynyl)pyridin-3-yl]-N- | |
| | (phenylmethyl)methanamine | |
| 428 | 1-[5-({2-Chloro-5-[5-(methylsulfonyl)-1-(3-morpholin-4-ylpropyl)-4,5,6,7- | 693.00 |
| | tetrahydro-1H-pyrazolo[4,3-c]pyridin-3-yl]phenyl}ethynyl)pyridin-3-yl]-N-[(4- | |
| | chlorophenyl)methyl]methanamine | |
| 429 | 2-{3-(4-Chloro-3-{[4-({[(4- | 760.30 |
| | chlorophenyl)methyl]amino}methyl)phenyl]ethynyl}phenyl)-1-[3-(4- | |
| | phenylpiperazin-1-yl)propyl]-1,4,6,7-tetrahydro-5H-pyrazolo[4,3-c]pyridin-5- | |
| | yl}-2-oxoacetamide | |
| 430 | 3-(4-Chloro-3-{[4-({[(4- | 732.30 |
| | chlorophenyl)methyl]amino}methyl)phenyl]ethynyl}phenyl)-1-[3-(4- | |
| | phenylpiperazin-1-yl)propyl]-1,4,6,7-tetrahydro-5H-pyrazolo[4,3-c]pyridine- | |
| | 5-carboxamide | |
| 431 | 1-[4-({2-Chloro-5-[1-{3-[(3S)-3-methylmorpholin-4-yl]propyl}-5- | 692.10 |
| | (methylsulfonyl)-4,5,6,7-tetrahydro-1H-pyrazolo[4,3-c]pyridin-3- | |
| | yl]phenyl}ethynyl)phenyl]-N-[(3-methyl-2-thienyl)methyl]methanamine | |
| 432 | 1-[4-({2-Chloro-5-[1-{3-[(3S)-3-methylmorpholin-4-yl]propyl}-5- | 662.10 |
| | (methylsulfonyl)-4,5,6,7-tetrahydro-1H-pyrazolo[4,3-c]pyridin-3- | |
| | yl]phenyl}ethynyl)phenyl]-N-(furan-2-ylmethyl)methanamine | |
| 433 | 1-[4-({2-Chloro-5-[1-{3-[(3S)-3-methylmorpholin-4-yl]propyl}-5- | 678.10 |
| | (methylsulfonyl)-4,5,6,7-tetrahydro-1H-pyrazolo[4,3-c]pyridin-3- | |
| | yl]phenyl}ethynyl)phenyl]-N-(2-thienylmethyl)methanamine | |
| 434 | 1-[4-({2-Chloro-5-[1-{3-[(3S)-3-methylmorpholin-4-yl]propyl}-5- | 678.10 |
| | (methylsulfonyl)-4,5,6,7-tetrahydro-1H-pyrazolo[4,3-c]pyridin-3- | |
| | yl]phenyl}ethynyl)phenyl]-N-(3-thienylmethyl)methanamine | |
| 435 | 2-[3-(4-Chloro-3-{[4-({[(4- | 697.24 |
| | chlorophenyl)methyl]amino}methyl)phenyl]ethynyl}phenyl)-1-{3-[(1S,4S)-2- | |
| | oxa-5-azabicyclo[2.2.1]hept-5-yl]propyl}-1,4,6,7-tetrahydro-5H-pyrazolo[4,3- | |
| | c]pyridin-5-yl]-2-oxoacetamide | |
| 436 | 1-[4-({2-Chloro-5-[5-(methylsulfonyl)-1-(3-morpholin-4-ylpropyl)-4,5,6,7- | 710.00 |
| | tetrahydro-1H-pyrazolo[4,3-c]pyridin-3-yl]phenyl}ethynyl)-2-fluorophenyl]-N- | |
| | [(4-chlorophenyl)methyl]methanamine | |
| 437 | 4-{3-[3-(4-Chloro-3-{[4-({[(4- | 736.00 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1- | |
| | yl]propyl}morpholine-3-carboxylic acid | |
| 438 | 1-[3-({2-Chloro-5-[5-(methylsulfonyl)-1-(3-morpholin-4-ylpropyl)-4,5,6,7- | 692.10 |
| | tetrahydro-1H-pyrazolo[4,3-c]pyridin-3-yl]phenyl}ethynyl)phenyl]-N-[(4- | |
| | chlorophenyl)methyl]methanamine | |
| 439 | 1-[3-({2-Chloro-5-[1-{3-[(3S)-3-methylmorpholin-4-yl]propyl}-5- | 706.10 |
| | (methylsulfonyl)-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 440 | 1-[3-({2-Chloro-5-[5-(methylsulfonyl)-1-{3-[(1S,4S)-2-oxa-5- | 704.10 |
| | azabicyclo[2.2.1]hept-5-yl]propyl}-4,5,6,7-tetrahydro-1H-pyrazolo[4,3- | |
| | c]pyridin-3-yl]phenyl}ethynyl)phenyl]-N-[(4- | |
| | chlorophenyl)methyl]methanamine | |
| 441 | 3-[4-Chloro-3-(1H-imidazol-4-ylethynyl)phenyl]-5-(methylsulfonyl)-1-(3- | 529.00 |
| | morpholin-4-ylpropyl)-4,5,6,7-tetrahydro-1H-pyrazolo[4,3-c]pyridine | |
| 442 | 2-{[4-({2-Chloro-5-[5-(methylsulfonyl)-1-(3-morpholin-4-ylpropyl)-4,5,6,7- | 684.20 |
| | tetrahydro-1H-pyrazolo[4,3-c]pyridin-3-yl]phenyl}ethynyl)phenyl]methyl}- | |
| | 1,2,3,4-tetrahydroisoquinoline | |
| 443 | 3-(4-Chloro-3-{[4-(1,3-dihydro-2H-isoindol-2- | 670.10 |
| | ylmethyl)phenyl]ethynyl}phenyl)-5-(methylsulfonyl)-1-(3-morpholin-4- | |
| | ylpropyl)-4,5,6,7-tetrahydro-1H-pyrazolo[4,3-c]pyridine | |
| 444 | (1R)—N-{[4-({2-Chloro-5-[5-(methylsulfonyl)-1-(3-morpholin-4-ylpropyl)- | 698.20 |
| | 4,5,6,7-tetrahydro-1H-pyrazolo[4,3-c]pyridin-3- | |
| | yl]phenyl}ethynyl)phenyl]methyl}-1,2,3,4-tetrahydronaphthalen-1-amine | |
| 445 | (1S,2R)-2-({[4-({2-Chloro-5-[5-(methylsulfonyl)-1-(3-morpholin-4-ylpropyl)- | 700.20 |
| | 4,5,6,7-tetrahydro-1H-pyrazolo[4,3-c]pyridin-3- | |
| | yl]phenyl}ethynyl)phenyl]methyl}amino)-2,3-dihydro-1H-inden-1-ol | |
| 446 | 1-{3-[(2-Chloro-5-{5-(methylsulfonyl)-1-[3-(4-phenylpiperazin-1-yl)propyl]- | 767.10 |
| | 4,5,6,7-tetrahydro-1H-pyrazolo[4,3-c]pyridin-3-yl}phenyl)ethynyl]phenyl}-N- | |
| | [(4-chlorophenyl)methyl]methanamine | |
| 447 | 1-{3-[(2-Chloro-5-{5-(methylsulfonyl)-1-[3-(4-pyridin-2-ylpiperazin-1- | 768.10 |
| | yl)propyl]-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 448 | (2S)-1-(4,4′-Bipiperidin-1-yl)-3-[3-{4-chloro-3-[(4- | 670.10 |
| | chlorophenyl)ethynyl]phenyl}-5-(methylsulfonyl)-4,5,6,7-tetrahydro-1H- | |
| | pyrazolo[4,3-c]pyridin-1-yl]propan-2-ol | |
| 449 | 2-[3-(4-Chloro-3-{[4-({[(4- | 683.26 |
| | chlorophenyl)methyl]amino}methyl)phenyl]ethynyl}phenyl)-1-(3-piperidin-1- | |
| | ylpropyl)-1,4,6,7-tetrahydro-5H-pyrazolo[4,3-c]pyridin-5-yl]-2-oxoacetamide | |
| 450 | 1-(3-{5-[Amino(oxo)acetyl]-3-(4-chloro-3-{[4-({[(4- | 727.30 |
| | chlorophenyl)methyl]amino}methyl)phenyl]ethynyl}phenyl)-4,5,6,7- | |
| | tetrahydro-1H-pyrazolo[4,3-c]pyridin-1-yl}propyl)piperidine-4-carboxamide | |
| 451 | 2-{3-(4-Chloro-3-{[4-({[(4- | 743.30 |
| | chlorophenyl)methyl]amino}methyl)phenyl]ethynyl}phenyl)-1-[3-(1,4-dioxa- | |
| | 8-azaspiro[4.5]dec-8-yl)propyl]-1,4,6,7-tetrahydro-5H-pyrazolo[4,3- | |
| | c]pyridin-5-yl}-2-oxoacetamide | |
| 452 | 2-[3-(4-Chloro-3-{[4-({[(4- | 753.30 |
| | chlorophenyl)methyl]amino}methyl)phenyl]ethynyl}phenyl)-1-{3-[4- | |
| | (trifluoromethyl)piperidin-1-yl]propyl}-1,4,6,7-tetrahydro-5H-pyrazolo[4,3- | |
| | c]pyridin-5-yl]-2-oxoacetamide | |
| 453 | 3-(4-Chloro-3-{[4-({[(4- | 641.20 |
| | chlorophenyl)methyl]amino}methyl)phenyl]ethynyl}phenyl)-1-(3-pyrrolidin-1- | |
| | ylpropyl)-1,4,6,7-tetrahydro-5H-pyrazolo[4,3-c]pyridine-5-carboxamide | |
| 454 | 3-(4-Chloro-3-{[4-({[(4- | 749.28 |
| | chlorophenyl)methyl]amino}methyl)phenyl]ethynyl}phenyl)-1-{3-[4-(1- | |
| | oxidopyridin-2-yl)piperazin-1-yl]propyl}-1,4,6,7-tetrahydro-5H-pyrazolo[4,3- | |
| | c]pyridine-5-carboxamide | |
| 455 | 3-(4-Chloro-3-{[4-({[(4- | 723.25 |
| | chlorophenyl)methyl]amino}methyl)phenyl]ethynyl}phenyl)-1-{3-[4- | |
| | (trifluoromethyl)piperidin-1-yl]propyl}-1,4,6,7-tetrahydro-5H-pyrazolo[4,3- | |
| | c]pyridine-5-carboxamide | |
| 456 | 1-[4-({2-Chloro-5-[5-(methylsulfonyl)-1-(3-morpholin-4-ylpropyl)-4,5,6,7- | 665.10 |
| | tetrahydro-1H-pyrazolo[4,3-c]pyridin-3-yl]phenyl}ethynyl)phenyl]-N-(1,3- | |
| | thiazol-2-ylmethyl)methanamine | |
| 457 | 1-[4-({2-Chloro-5-[5-(methylsulfonyl)-1-(3-morpholin-4-ylpropyl)-4,5,6,7- | 662.20 |
| | tetrahydro-1H-pyrazolo[4,3-c]pyridin-3-yl]phenyl}ethynyl)phenyl]-N-[(1- | |
| | methyl-1H-imidazol-5-yl)methyl]methanamine | |
| 458 | 3-(4-Chloro-3-{[4-({[(4- | 655.26 |
| | chlorophenyl)methyl]amino}methyl)phenyl]ethynyl}phenyl)-1-(3-piperidin-1- | |
| | ylpropyl)-1,4,6,7-tetrahydro-5H-pyrazolo[4,3-c]pyridine-5-carboxamide | |
| 459 | 1-{3-[4-(Acetylamino)piperidin-1-yl]propyl}-3-(4-chloro-3-{[4-({[(4- | 712.29 |
| | chlorophenyl)methyl]amino}methyl)phenyl]ethynyl}phenyl)-1,4,6,7- | |
| | tetrahydro-5H-pyrazolo[4,3-c]pyridine-5-carboxamide | |
| 460 | 3-(4-Chloro-3-{[4-({[(4- | 710.31 |
| | chlorophenyl)methyl]amino}methyl)phenyl]ethynyl}phenyl)-1-[3-(4- | |
| | cyclobutylpiperazin-1-yl)propyl]-1,4,6,7-tetrahydro-5H-pyrazolo[4,3- | |
| | c]pyridine-5-carboxamide | |
| 461 | 1-{3-[4-(Aminocarbonyl)piperidin-1-yl]propyl}-3-(4-chloro-3-{[4-({[(4- | 700.30 |
| | chlorophenyl)methyl]amino}methyl)phenyl]ethynyl}phenyl)-1,4,6,7- | |
| | tetrahydro-5H-pyrazolo[4,3-c]pyridine-5-carboxamide | |
| 462 | 3-(4-Chloro-3-{[4-({[(4- | 713.27 |
| | chlorophenyl)methyl]amino}methyl)phenyl]ethynyl}phenyl)-1-[3-(1,4-dioxa- | |
| | 8-azaspiro[4.5]dec-8-yl)propyl]-1,4,6,7-tetrahydro-5H-pyrazolo[4,3- | |
| | c]pyridine-5-carboxamide | |
| 463 | 3-(4-Chloro-3-{[4-({[(4- | 696.29 |
| | chlorophenyl)methyl]amino}methyl)phenyl]ethynyl}phenyl)-1-[3-(4- | |
| | cyclopropylpiperazin-1-yl)propyl]-1,4,6,7-tetrahydro-5H-pyrazolo[4,3- | |
| | c]pyridine-5-carboxamide | |
| 464 | (3S)-7-({2-Chloro-5-[5-(methylsulfonyl)-1-(3-morpholin-4-ylpropyl)-4,5,6,7- | 738.10 |
| | tetrahydro-1H-pyrazolo[4,3-c]pyridin-3-yl]phenyl}ethynyl)-2-{[(1,1- | |
| | dimethylethyl)oxy]carbonyl}-1,2,3,4-tetrahydroisoquinoline-3-carboxylic acid | |
| 465 | 3-[4-Chloro-3-({2-[(4-chlorophenyl)methyl]-1,2,3,4-tetrahydroisoquinolin-7- | 683.20 |
| | yl}ethynyl)phenyl]-1-(3-morpholin-4-ylpropyl)-1,4,6,7-tetrahydro-5H- | |
| | pyrazolo[4,3-c]pyridine-5-carboxamide | |
| 466 | (3S)-7-({2-Chloro-5-[5-(methylsulfonyl)-1-(3-morpholin-4-ylpropyl)-4,5,6,7- | 691.20 |
| | tetrahydro-1H-pyrazolo[4,3-c]pyridin-3-yl]phenyl}ethynyl)-3-(pyrrolidin-1- | |
| | ylcarbonyl)-1,2,3,4-tetrahydroisoquinoline | |
| 467 | 1,1-Dimethylethyl 7-({2-chloro-5-[1-{3-[(3S)-3-methylmorpholin-4-yl]propyl}- | 708.20 |
| | 5-(methylsulfonyl)-4,5,6,7-tetrahydro-1H-pyrazolo[4,3-c]pyridin-3- | |
| | yl]phenyl}ethynyl)-3,4-dihydroisoquinoline-2(1H)-carboxylate | |
| 468 | 1-{4-[(2-Chloro-5-{5-(methylsulfonyl)-1-[3-(2-oxa-6-azaspiro[3.3]hept-6- | 704.10 |
| | yl)propyl]-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 469 | 2-({[4-({2-Chloro-5-[5-(methylsulfonyl)-1-(3-morpholin-4-ylpropyl)-4,5,6,7- | 612.20 |
| | tetrahydro-1H-pyrazolo[4,3-c]pyridin-3- | |
| | yl]phenyl}ethynyl)phenyl]methyl}amino)ethanol | |
| 470 | N-({4-[(2-Chloro-5-{5-(methylsulfonyl)-1-[3-(4-pyridin-2-ylpiperazin-1- | 760.20 |
| | yl)propyl]-4,5,6,7-tetrahydro-1H-pyrazolo[4,3-c]pyridin-3- | |
| | yl}phenyl)ethynyl]phenyl}methyl)-2,3-dihydro-1H-inden-1-amine | |
| 471 | (2S)-1-(4,4′-Bipiperidin-1-yl)-3-[3-{4-chloro-3-[3-(diethylamino)prop-1-yn-1- | 745.30 |
| | yl]phenyl}-5-(methylsulfonyl)-4,5,6,7-tetrahydro-1H-pyrazolo[4,3-c]pyridin-1- | |
| | yl]propan-2-ol | |
| 472 | (2S)-1-(4,4′-Bipiperidin-1-yl)-3-{3-[4-chloro-3-(cyclohexylethynyl)phenyl]-5- | 642.30 |
| | (methylsulfonyl)-4,5,6,7-tetrahydro-1H-pyrazolo[4,3-c]pyridin-1-yl}propan-2- | |
| | ol | |
| 473 | (2S)-1-(4,4′-Bipiperidin-1-yl)-3-{3-[4-chloro-3-(pyridin-2-ylethynyl)phenyl]-5- | 637.20 |
| | (methylsulfonyl)-4,5,6,7-tetrahydro-1H-pyrazolo[4,3-c]pyridin-1-yl}propan-2- | |
| | ol | |
| 474 | (2S)-1-(4,4′-Bipiperidin-1-yl)-3-{3-[4-chloro-3-(3-phenylprop-1-yn-1- | 650.20 |
| | yl)phenyl]-5-(methylsulfonyl)-4,5,6,7-tetrahydro-1H-pyrazolo[4,3-c]pyridin-1- | |
| | yl}propan-2-ol | |
| 475 | (2S)-1-(4,4′-Bipiperidin-1-yl)-3-{3-[4-chloro-3-(pyridin-3-ylethynyl)phenyl]-5- | 637.20 |
| | (methylsulfonyl)-4,5,6,7-tetrahydro-1H-pyrazolo[4,3-c]pyridin-1-yl}propan-2- | |
| | ol | |
| 476 | 3-(4-Chloro-3-{2-[4-({[(4- | 661.28 |
| | chlorophenyl)methyl]amino}methyl)phenyl]ethyl}phenyl)-1-(3-morpholin-4- | |
| | ylpropyl)-1,4,6,7-tetrahydro-5H-pyrazolo[4,3-c]pyridine-5-carboxamide | |
| 477 | 3-{4-Chloro-3-[(4-{[(1R)-1,2,3,4-tetrahydronaphthalen-1- | 663.20 |
| | ylamino]methyl}phenyl)ethynyl]phenyl}-1-(3-morpholin-4-ylpropyl)-1,4,6,7- | |
| | tetrahydro-5H-pyrazolo[4,3-c]pyridine-5-carboxamide | |
| 478 | N-({4-[(2-Chloro-5-{5-(methylsulfonyl)-1-[3-(4-phenylpiperazin-1-yl)propyl]- | 747.20 |
| | 4,5,6,7-tetrahydro-1H-pyrazolo[4,3-c]pyridin-3- | |
| | yl}phenyl)ethynyl]phenyl}methyl)-1-phenylethanamine | |
| 479 | 1-[(2S)-3-(4,4′-Bipiperidin-1-yl)-2-hydroxypropyl]-3-{4-chloro-3-[2-(4- | 639.20 |
| | chlorophenyl)ethyl]phenyl}-1,4,6,7-tetrahydro-5H-pyrazolo[4,3-c]pyridine-5- | |
| | carboxamide | |
| 480 | N-({4-[(2-Chloro-5-{5-(methylsulfonyl)-1-[3-(4-phenylpiperazin-1-yl)propyl]- | 725.20 |
| | 4,5,6,7-tetrahydro-1H-pyrazolo[4,3-c]pyridin-3- | |
| | yl}phenyl)ethynyl]phenyl}methyl)-2,2,2-trifluoroethanamine | |
| 481 | 7-{[2-Chloro-5-(1-{3-[(3S)-3-methylmorpholin-4-yl]propyl}-4,5,6,7- | 570.30 |
| | tetrahydro-1H-pyrazolo[4,3-c]pyridin-3-yl)phenyl]ethynyl}-2-cyclopropyl- | |
| | 1,2,3,4-tetrahydroisoquinoline | |
| 482 | 3-[4-Chloro-3-(1H-pyrrolo[2,3-b]pyridin-6-ylethynyl)phenyl]-5- | 579.10 |
| | (methylsulfonyl)-1-(3-morpholin-4-ylpropyl)-4,5,6,7-tetrahydro-1H- | |
| | pyrazolo[4,3-c]pyridine | |
| 483 | 6-({2-Chloro-5-[5-(methylsulfonyl)-1-(3-morpholin-4-ylpropyl)-4,5,6,7- | 707.20 |
| | tetrahydro-1H-pyrazolo[4,3-c]pyridin-3-yl]phenyl}ethynyl)-3-(morpholin-4- | |
| | ylcarbonyl)-1,2,3,4-tetrahydroisoquinoline | |
| 484 | 6-({2-Chloro-5-[5-(methylsulfonyl)-1-(3-morpholin-4-ylpropyl)-4,5,6,7- | 691.20 |
| | tetrahydro-1H-pyrazolo[4,3-c]pyridin-3-yl]phenyl}ethynyl)-3-(pyrrolidin-1- | |
| | ylcarbonyl)-1,2,3,4-tetrahydroisoquinoline | |
| 485 | (3S)-7-({2-Chloro-5-[5-(methylsulfonyl)-1-(3-morpholin-4-ylpropyl)-4,5,6,7- | 720.20 |
| | tetrahydro-1H-pyrazolo[4,3-c]pyridin-3-yl]phenyl}ethynyl)-3-[(4- | |
| | methylpiperazin-1-yl)carbonyl]-1,2,3,4-tetrahydroisoquinoline | |
| 486 | (3S)-7-({2-Chloro-5-[5-(methylsulfonyl)-1-(3-morpholin-4-ylpropyl)-4,5,6,7- | 705.20 |
| | tetrahydro-1H-pyrazolo[4,3-c]pyridin-3-yl]phenyl}ethynyl)-3-(piperidin-1- | |
| | ylcarbonyl)-1,2,3,4-tetrahydroisoquinoline | |
| 487 | 3-(4-Chloro-3-{[4-({[(3- | 673.30 |
| | chlorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 488 | 3-(4-Chloro-3-{[4-({[(2- | 673.30 |
| | chlorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 489 | 8-({2-Chloro-5-[5-(methylsulfonyl)-1-(3-morpholin-4-ylpropyl)-4,5,6,7- | 608.20 |
| | tetrahydro-1H-pyrazolo[4,3-c]pyridin-3-yl]phenyl}ethynyl)-2,3,4,5- | |
| | tetrahydro-1H-2-benzazepine | |
| 490 | 1-{4-[(2-Chloro-5-{5-(methylsulfonyl)-1-[3-(4-phenylpiperazin-1-yl)propyl]- | 781.20 |
| | 4,5,6,7-tetrahydro-1H-pyrazolo[4,3-c]pyridin-3-yl}phenyl)ethynyl]phenyl}-N- | |
| | [(4-chlorophenyl)methyl]-N-methylmethanamine | |
| 491 | 1,1-Dimethylethyl (1S,4S)-5-{3-[3-(4-chloro-3-{[4-({[(4- | 803.20 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]propyl}- | |
| | 2,5-diazabicyclo[2.2.1]heptane-2-carboxylate | |
| 492 | 1,1-Dimethylethyl (1R,4R)-5-{3-[3-(4-chloro-3-{[4-({[(4- | 803.20 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]propyl}- | |
| | 2,5-diazabicyclo[2.2.1]heptane-2-carboxylate | |
| 493 | 1-[4-({2-Chloro-5-[1-{3-[(1S,4S)-2,5-diazabicyclo[2.2.1]hept-2-yl]propyl}-5- | 703.20 |
| | (methylsulfonyl)-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 494 | 1-[4-({2-Chloro-5-[1-{3-[(1R,4R)-2,5-diazabicyclo[2.2.1]hept-2-yl]propyl}-5- | 703.20 |
| | (methylsulfonyl)-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 495 | 1-[4-({2-Chloro-5-[5-(methylsulfonyl)-1-(3-morpholin-4-ylpropyl)-4,5,6,7- | 706.20 |
| | tetrahydro-1H-pyrazolo[4,3-c]pyridin-3-yl]phenyl}ethynyl)phenyl]-N-[(4- | |
| | chlorophenyl)methyl]-N-methylmethanamine | |
| 496 | 2-[3-{4-Chloro-3-[(4-{[(1R)-1,2,3,4-tetrahydronaphthalen-1- | 691.30 |
| | ylamino]methyl}phenyl)ethynyl]phenyl}-1-(3-morpholin-4-ylpropyl)-1,4,6,7- | |
| | tetrahydro-5H-pyrazolo[4,3-c]pyridin-5-yl]-2-oxoacetamide | |
| 497 | 1-[4-({2-Chloro-5-[5-(morpholin-4-ylcarbonyl)-1-(3-morpholin-4-ylpropyl)- | 729.30 |
| | 4,5,6,7-tetrahydro-1H-pyrazolo[4,3-c]pyridin-3-yl]phenyl}ethynyl)phenyl]-N- | |
| | [(4-chlorophenyl)methyl]methanamine | |
| 498 | 3-(4-Chloro-3-{[4-({[(4- | 687.30 |
| | chlorophenyl)methyl]amino}methyl)phenyl]ethynyl}phenyl)-N,N-dimethyl-1- | |
| | (3-morpholin-4-ylpropyl)-1,4,6,7-tetrahydro-5H-pyrazolo[4,3-c]pyridine-5- | |
| | carboxamide | |
| 499 | N-{2-[3-(4-Chloro-3-{[4-({[(4- | 713.27 |
| | chlorophenyl)methyl]amino}methyl)phenyl]ethynyl}phenyl)-1-(3-morpholin- | |
| | 4-ylpropyl)-1,4,6,7-tetrahydro-5H-pyrazolo[4,3-c]pyridin-5-yl]-2- | |
| | oxoethyl}acetamide | |
| 500 | 1-[4-({2-Chloro-5-[1-{3-[(3S)-3-methylmorpholin-4-yl]propyl}-5- | 720.20 |
| | (methylsulfonyl)-4,5,6,7-tetrahydro-1H-pyrazolo[4,3-c]pyridin-3- | |
| | yl]phenyl}ethynyl)phenyl]-N-[(4-chlorophenyl)methyl]-N- | |
| | methylmethanamine | |
| 501 | 7-({2-Chloro-5-[1-{3-[(3S)-3-methylmorpholin-4-yl]propyl}-5- | 690.20 |
| | (methylsulfonyl)-4,5,6,7-tetrahydro-1H-pyrazolo[4,3-c]pyridin-3- | |
| | yl]phenyl}ethynyl)-2-(2,2,2-trifluoroethyl)-1,2,3,4-tetrahydroisoquinoline | |
| 502 | 1-{4-[(2-Chloro-5-{5-(methylsulfonyl)-1-[3-(4-pyridin-2-ylpiperazin-1- | 782.20 |
| | yl)propyl]-4,5,6,7-tetrahydro-1H-pyrazolo[4,3-c]pyridin-3- | |
| | yl}phenyl)ethynyl]phenyl}-N-[(4-chlorophenyl)methyl]-N- | |
| | methylmethanamine | |
| 503 | 1-[4-({2-Chloro-5-[5-(methylsulfonyl)-1-(3-thiomorpholin-4-ylpropyl)-4,5,6,7- | 722.10 |
| | tetrahydro-1H-pyrazolo[4,3-c]pyridin-3-yl]phenyl}ethynyl)phenyl]-N-[(4- | |
| | chlorophenyl)methyl]-N-methylmethanamine | |
| 504 | N-{[3-({2-Chloro-5-[5-(methylsulfonyl)-1-(3-morpholin-4-ylpropyl)-4,5,6,7- | 672.20 |
| | tetrahydro-1H-pyrazolo[4,3-c]pyridin-3-yl]phenyl}ethynyl)phenyl]methyl}-2- | |
| | phenylethanamine | |
| 505 | 6-({2-Chloro-5-[5-(methylsulfonyl)-1-(3-morpholin-4-ylpropyl)-4,5,6,7- | 648.20 |
| | tetrahydro-1H-pyrazolo[4,3-c]pyridin-3-yl]phenyl}ethynyl)-2- | |
| | (cyclopropylmethyl)-1,2,3,4-tetrahydroisoquinoline | |
| 506 | 2-({[3-({2-Chloro-5-[5-(methylsulfonyl)-1-(3-morpholin-4-ylpropyl)-4,5,6,7- | 612.20 |
| | tetrahydro-1H-pyrazolo[4,3-c]pyridin-3- | |
| | yl]phenyl}ethynyl)phenyl]methyl}amino)ethanol | |
| 507 | N-{[3-({2-Chloro-5-[5-(methylsulfonyl)-1-(3-morpholin-4-ylpropyl)-4,5,6,7- | 672.20 |
| | tetrahydro-1H-pyrazolo[4,3-c]pyridin-3-yl]phenyl}ethynyl)phenyl]methyl}-1- | |
| | phenylethanamine | |
| 508 | 3-(4-Chloro-3-{[4-({[(3- | 653.30 |
| | methylphenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 509 | 3-({[3-({2-Chloro-5-[5-(methylsulfonyl)-1-(3-morpholin-4-ylpropyl)-4,5,6,7- | 626.20 |
| | tetrahydro-1H-pyrazolo[4,3-c]pyridin-3- | |
| | yl]phenyl}ethynyl)phenyl]methyl}amino)propan-1-ol | |
| 510 | 1-[3-({2-Chloro-5-[5-(methylsulfonyl)-1-(3-morpholin-4-ylpropyl)-4,5,6,7- | 652.20 |
| | tetrahydro-1H-pyrazolo[4,3-c]pyridin-3-yl]phenyl}ethynyl)phenyl]-N- | |
| | (tetrahydrofuran-2-ylmethyl)methanamine | |
| 511 | N-{[3-({2-Chloro-5-[5-(methylsulfonyl)-1-(3-morpholin-4-ylpropyl)-4,5,6,7- | 644.20 |
| | tetrahydro-1H-pyrazolo[4,3-c]pyridin-3- | |
| | yl]phenyl}ethynyl)phenyl]methyl}aniline | |
| 512 | 6-({2-Chloro-5-[1-{3-[(3S)-3-methylmorpholin-4-yl]propyl}-5- | 648.20 |
| | (methylsulfonyl)-4,5,6,7-tetrahydro-1H-pyrazolo[4,3-c]pyridin-3- | |
| | yl]phenyl}ethynyl)-2-cyclopropyl-1,2,3,4-tetrahydroisoquinoline | |
| 513 | 3-(4-Chloro-3-{[3-({[(2- | 673.22 |
| | chlorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 514 | 3-(4-Chloro-3-{[3-({[(3- | 673.22 |
| | chlorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 515 | 3-(4-Chloro-3-{[3-({[(2- | 657.30 |
| | fluorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 516 | 3-(4-Chloro-3-{[3-({[(3- | 657.25 |
| | fluorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 517 | 3-(4-Chloro-3-{[3-({[(4- | 657.25 |
| | fluorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 518 | 6-({2-Chloro-5-[5-(methylsulfonyl)-1-(3-morpholin-4-ylpropyl)-4,5,6,7- | 608.20 |
| | tetrahydro-1H-pyrazolo[4,3-c]pyridin-3-yl]phenyl}ethynyl)-2-methyl-1,2,3,4- | |
| | tetrahydroisoquinoline | |
| 519 | 6-({2-Chloro-5-[1-{3-[(3S)-3-methylmorpholin-4-yl]propyl}-5- | 622.20 |
| | (methylsulfonyl)-4,5,6,7-tetrahydro-1H-pyrazolo[4,3-c]pyridin-3- | |
| | yl]phenyl}ethynyl)-2-methyl-1,2,3,4-tetrahydroisoquinoline | |
| 520 | N-{[3-({2-Chloro-5-[5-(methylsulfonyl)-1-(3-morpholin-4-ylpropyl)-4,5,6,7- | 624.20 |
| | tetrahydro-1H-pyrazolo[4,3-c]pyridin-3-yl]phenyl}ethynyl)phenyl]methyl}-2- | |
| | methylpropan-1-amine | |
| 521 | 1-[3-({2-Chloro-5-[5-(methylsulfonyl)-1-(3-morpholin-4-ylpropyl)-4,5,6,7- | 659.20 |
| | tetrahydro-1H-pyrazolo[4,3-c]pyridin-3-yl]phenyl}ethynyl)phenyl]-N-(pyridin- | |
| | 3-ylmethyl)methanamine | |
| 522 | 1-[3-({2-Chloro-5-[5-(methylsulfonyl)-1-(3-morpholin-4-ylpropyl)-4,5,6,7- | 659.20 |
| | tetrahydro-1H-pyrazolo[4,3-c]pyridin-3-yl]phenyl}ethynyl)phenyl]-N-(pyridin- | |
| | 4-ylmethyl)methanamine | |
| 523 | 6-({2-Chloro-5-[5-(methylsulfonyl)-1-(3-morpholin-4-ylpropyl)-4,5,6,7- | 650.20 |
| | tetrahydro-1H-pyrazolo[4,3-c]pyridin-3-yl]phenyl}ethynyl)-2-(2- | |
| | methylpropyl)-1,2,3,4-tetrahydroisoquinoline | |
| 524 | 1-[3-({2-Chloro-5-[5-(methylsulfonyl)-1-(3-morpholin-4-ylpropyl)-4,5,6,7- | 736.20 |
| | tetrahydro-1H-pyrazolo[4,3-c]pyridin-3-yl]phenyl}ethynyl)phenyl]-N-{[4- | |
| | (methylsulfonyl)phenyl]methyl}methanamine | |
| 525 | 1,1-Dimethylethyl 6-({2-chloro-5-[5-(methylsulfonyl)-1-(3-morpholin-4- | 708.20 |
| | ylpropyl)-4,5,6,7-tetrahydro-1H-pyrazolo[4,3-c]pyridin-3-yl]phenyl}ethynyl)- | |
| | 1-oxo-3,4-dihydroisoquinoline-2(1H)-carboxylate | |
| 526 | Ethyl [6-({2-chloro-5-[5-(methylsulfonyl)-1-(3-morpholin-4-ylpropyl)-4,5,6,7- | 680.20 |
| | tetrahydro-1H-pyrazolo[4,3-c]pyridin-3-yl]phenyl}ethynyl)-3,4- | |
| | dihydroisoquinolin-2(1H)-yl]acetate | |
| 527 | 6-({2-Chloro-5-[5-(methylsulfonyl)-1-(3-morpholin-4-ylpropyl)-4,5,6,7- | 634.20 |
| | tetrahydro-1H-pyrazolo[4,3-c]pyridin-3-yl]phenyl}ethynyl)-2-prop-2-en-1-yl- | |
| | 1,2,3,4-tetrahydroisoquinoline | |
| 528 | 1-[3-({2-Chloro-5-[5-(methylsulfonyl)-1-(3-morpholin-4-ylpropyl)-4,5,6,7- | 672.20 |
| | tetrahydro-1H-pyrazolo[4,3-c]pyridin-3-yl]phenyl}ethynyl)phenyl]-N-methyl- | |
| | N-(phenylmethyl)methanamine | |
| 529 | (2R)-1-[3-(4-Chloro-3-{[4-({[(4- | 722.10 |
| | chlorophenyl)methyl]amino}methyl)phenyl]ethynyl}phenyl)-5- | |
| | (methylsulfonyl)-4,5,6,7-tetrahydro-1H-pyrazolo[4,3-c]pyridin-1-yl]-3-[(3S)- | |
| | 3-methylmorpholin-4-yl]propan-2-ol | |
| 530 | 1-[3-(2-{2-Chloro-5-[5-(methylsulfonyl)-1-(3-morpholin-4-ylpropyl)-4,5,6,7- | 676.30 |
| | tetrahydro-1H-pyrazolo[4,3-c]pyridin-3-yl]phenyl}ethyl)phenyl]-N-methyl-N- | |
| | (phenylmethyl)methanamine | |
| 531 | N-{[3-(2-{2-Chloro-5-[5-(methylsulfonyl)-1-(3-morpholin-4-ylpropyl)-4,5,6,7- | 628.20 |
| | tetrahydro-1H-pyrazolo[4,3-c]pyridin-3-yl]phenyl}ethyl)phenyl]methyl}-2- | |
| | methylpropan-1-amine | |
| 532 | 3-(4-Chloro-3-{[4-({[(2- | 653.30 |
| | methylphenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 533 | 3-[4-Chloro-3-({4-[({[2- | 669.30 |
| | (methyloxy)phenyl]methyl}amino)methyl]phenyl}ethynyl)phenyl]-1-(3- | |
| | thiomorpholin-4-ylpropyl)-1,4,6,7-tetrahydro-5H-pyrazolo[4,3-c]pyridine-5- | |
| | carboxamide | |
| 534 | 3-[4-Chloro-3-({4-[({[3- | 669.30 |
| | (methyloxy)phenyl]methyl}amino)methyl]phenyl}ethynyl)phenyl]-1-(3- | |
| | thiomorpholin-4-ylpropyl)-1,4,6,7-tetrahydro-5H-pyrazolo[4,3-c]pyridine-5- | |
| | carboxamide | |
| 535 | 1-[3-(2-{2-Chloro-5-[5-(methylsulfonyl)-1-(3-morpholin-4-ylpropyl)-4,5,6,7- | 662.20 |
| | tetrahydro-1H-pyrazolo[4,3-c]pyridin-3-yl]phenyl}ethyl)phenyl]-N- | |
| | (phenylmethyl)methanamine | |
| 536 | 1-[4-({2-Chloro-5-[1-{3-[(3R)-3-methylmorpholin-4-yl]propyl}-5- | 706.20 |
| | (methylsulfonyl)-4,5,6,7-tetrahydro-1H-pyrazolo[4,3-c]pyridin-3- | |
| | yl]phenyl}ethynyl)phenyl]-N-[(4-chlorophenyl)methyl]methanamine | |
| 537 | 1-{4-[(2-Chloro-5-{5-(methylsulfonyl)-1-[3-(1-oxidothiomorpholin-4- | 724.10 |
| | yl)propyl]-4,5,6,7-tetrahydro-1H-pyrazolo[4,3-c]pyridin-3- | |
| | yl}phenyl)ethynyl]phenyl}-N-[(4-chlorophenyl)methyl]methanamine | |
| 538 | 3-[4-Chloro-3-({4-[({[4- | 669.30 |
| | (methyloxy)phenyl]methyl}amino)methyl]phenyl}ethynyl)phenyl]-1-(3- | |
| | thiomorpholin-4-ylpropyl)-1,4,6,7-tetrahydro-5H-pyrazolo[4,3-c]pyridine-5- | |
| | carboxamide | |
| 539 | 1-[5-({2-Chloro-5-[1-{3-[(3S)-3-methylmorpholin-4-yl]propyl}-5- | 690.20 |
| | (methylsulfonyl)-4,5,6,7-tetrahydro-1H-pyrazolo[4,3-c]pyridin-3- | |
| | yl]phenyl}ethynyl)-2-fluorophenyl]-N-(phenylmethyl)methanamine | |
| 540 | 1-[5-({2-Chloro-5-[5-(methylsulfonyl)-1-(3-morpholin-4-ylpropyl)-4,5,6,7- | 688.20 |
| | tetrahydro-1H-pyrazolo[4,3-c]pyridin-3-yl]phenyl}ethynyl)-2- | |
| | (methyloxy)phenyl]-N-(phenylmethyl)methanamine | |
| 541 | (3R)-7-({2-Chloro-5-[5-(methylsulfonyl)-1-(3-morpholin-4-ylpropyl)-4,5,6,7- | 707.20 |
| | tetrahydro-1H-pyrazolo[4,3-c]pyridin-3-yl]phenyl}ethynyl)-3-(morpholin-4- | |
| | ylcarbonyl)-1,2,3,4-tetrahydroisoquinoline | |
| 542 | 3-(4-Chloro-3-{[4-({[(2- | 657.20 |
| | fluorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 543 | 3-(4-Chloro-3-{[4-({[(3- | 357.20 |
| | fluorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 544 | 3-(4-Chloro-3-{[4-({[(4- | 657.20 |
| | fluorophenyl)methyl]amino}methyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 545 | 1-[2-Chloro-4-({2-chloro-5-[5-(methylsulfonyl)-1-(3-morpholin-4-ylpropyl)- | 692.20 |
| | 4,5,6,7-tetrahydro-1H-pyrazolo[4,3-c]pyridin-3-yl]phenyl}ethynyl)phenyl]-N- | |
| | (phenylmethyl)methanamine | |
| 546 | 1-[2-Chloro-4-({2-chloro-5-[5-(methylsulfonyl)-1-(3-morpholin-4-ylpropyl)- | 726.10 |
| | 4,5,6,7-tetrahydro-1H-pyrazolo[4,3-c]pyridin-3-yl]phenyl}ethynyl)phenyl]-N- | |
| | [(4-chlorophenyl)methyl]methanamine | |
| 547 | 3-(4-Chloro-3-{[4-({[(4- | 687.20 |
| | chlorophenyl)methyl]amino}carbonyl)phenyl]ethynyl}phenyl)-1-(3- | |
| | thiomorpholin-4-ylpropyl)-1,4,6,7-tetrahydro-5H-pyrazolo[4,3-c]pyridine-5- | |
| | carboxamide | |
| 548 | 3-{4-Chloro-3-[(4-{[(morpholin-2- | 649.20 |
| | ylmethyl)oxy]methyl}phenyl)ethynyl]phenyl}-1-(3-thiomorpholin-4-ylpropyl)- | |
| | 1,4,6,7-tetrahydro-5H-pyrazolo[4,3-c]pyridine-5-carboxamide | |
| 549 | 3-(4-Chloro-3-{[4-({4-[(4R)-4-hydroxy-2-oxopyrrolidin-1-yl]piperidin-1- | 716.31 |
| | yl}methyl)phenyl]ethynyl}phenyl)-1-(3-thiomorpholin-4-ylpropyl)-1,4,6,7- | |
| | tetrahydro-5H-pyrazolo[4,3-c]pyridine-5-carboxamide | |
| 550 | 3-[4-Chloro-3-({4-[({[4- | 685.20 |
| | (methylsulfanyl)phenyl]methyl}amino)methyl]phenyl}ethynyl)phenyl]-1-(3- | |
| | thiomorpholin-4-ylpropyl)-1,4,6,7-tetrahydro-5H-pyrazolo[4,3-c]pyridine-5- | |
| | carboxamide | |
| 551 | 3-(3-{[4-({[(4-Aminophenyl)methyl]amino}methyl)phenyl]ethynyl}-4- | 655.20 |
| | chlorophenyl)-1-(3-thiomorpholin-4-ylpropyl)-1,4,6,7-tetrahydro-5H- | |
| | pyrazolo[4,3-c]pyridine-5-carboxamide | |
| 552 | 3-{4-Chloro-3-[(4-{[(3R)-3-hydroxypyrrolidin-1- | 633.23 |
| | yl]carbonyl}phenyl)ethynyl]phenyl}-1-(3-thiomorpholin-4-ylpropyl)-1,4,6,7- | |
| | tetrahydro-5H-pyrazolo[4,3-c]pyridine-5-carboxamide | |
| 553 | N-[(2-Chloro-5-{[2-chloro-5-(1-{3-[(3S)-3-methylmorpholin-4-yl]propyl}- | 595.90 |
| | 4,5,6,7-tetrahydro-1H-pyrazolo[4,3-c]pyridin-3- | |
| | yl)phenyl]ethynyl}phenyl)methyl]glycine | |
| 554 | 5-[(5-{5-[Amino(oxo)acetyl]-1-(3-thiomorpholin-4-ylpropyl)-4,5,6,7- | 684.20 |
| | tetrahydro-1H-pyrazolo[4,3-c]pyridin-3-yl}-2-chlorophenyl)ethynyl]-2-chloro- | |
| | N-(3-hydroxypropyl)benzamide | |
| 555 | 3-[4-Chloro-3-({4-chloro-3-[(3- | 656.20 |
| | hydroxypropyl)carbamoyl]phenyl}ethynyl)phenyl]-1-(3-thiomorpholin-4- | |
| | ylpropyl)-1,4,6,7-tetrahydro-5H-pyrazolo[4,3-c]pyridine-5-carboxamide | |
| 556 | 5-{[5-(5-[Amino(oxo)acetyl]-1-{3-[(3S)-3-methylmorpholin-4-yl]propyl}- | 707.30 |
| | 4,5,6,7-tetrahydro-1H-pyrazolo[4,3-c]pyridin-3-yl)-2-chlorophenyl]ethynyl}- | |
| | 2-chloro-N-[(2S)-pyrrolidin-2-ylmethyl]benzamide | |
| 557 | 5-[(5-{5-[Amino(oxo)acetyl]-1-(3-thiomorpholin-4-ylpropyl)-4,5,6,7- | 710.20 |
| | tetrahydro-1H-pyrazolo[4,3-c]pyridin-3-yl}-2-chlorophenyl)ethynyl]-2-chloro- | |
| | N-[(2S)-pyrrolidin-2-ylmethyl]benzamide | |
| 558 | 3-{4-Chloro-3-[(4-chloro-3-{[(2S)-pyrrolidin-2- | 682.20 |
| | ylmethyl]carbamoyl}phenyl)ethynyl]phenyl}-1-(3-thiomorpholin-4-ylpropyl)- | |
| | 1,4,6,7-tetrahydro-5H-pyrazolo[4,3-c]pyridine-5-carboxamide | |
| 559 | tert-Butyl 3-[(5-{[5-(5-[amino(oxo)acetyl]-1-{3-[(3S)-3-methylmorpholin-4- | 779.30 |
| | yl]propyl}-4,5,6,7-tetrahydro-1H-pyrazolo[4,3-c]pyridin-3-yl)-2- | |
| | chlorophenyl]ethynyl}-2-chlorobenzyl)oxy]pyrrolidine-1-carboxylate | |
| 560 | 5-{[5-(5-[Amino(oxo)acetyl]-1-{3-[(3S)-3-methylmorpholin-4-yl]propyl}- | 683.20 |
| | 4,5,6,7-tetrahydro-1H-pyrazolo[4,3-c]pyridin-3-yl)-2-chlorophenyl]ethynyl}- | |
| | 2-chloro-N-(3-hydroxypropyl)benzamide | |
| 561 | 2-(3-{4-Chloro-3-[(4-chloro-3-{[(2-hydroxy-2- | 681.20 |
| | methylpropyl)amino]methyl}phenyl)ethynyl]phenyl}-1-{3-[(3S)-3- | |
| | methylmorpholin-4-yl]propyl}-1,4,6,7-tetrahydro-5H-pyrazolo[4,3-c]pyridin- | |
| | 5-yl)-2-oxoacetamide | |
| 562 | 2-[3-{4-Chloro-3-[(4-chloro-3-{[(2-hydroxy-2- | 683.23 |
| | methylpropyl)amino]methyl}phenyl)ethynyl]phenyl}-1-(3-thiomorpholin-4- | |
| | ylpropyl)-1,4,6,7-tetrahydro-5H-pyrazolo[4,3-c]pyridin-5-yl]-2-oxoacetamide | |
| 563 | 2-[3-(4-Chloro-3-{[4-chloro-3-({[(1R)-2-hydroxy-1- | 731.20 |
| | phenylethyl]amino}methyl)phenyl]ethynyl}phenyl)-1-(3-thiomorpholin-4- | |
| | ylpropyl)-1,4,6,7-tetrahydro-5H-pyrazolo[4,3-c]pyridin-5-yl]-2-oxoacetamide | |
| 564 | 5-[(5-{5-[Amino(oxo)acetyl]-1-(3-thiomorpholin-4-ylpropyl)-4,5,6,7- | 745.21 |
| | tetrahydro-1H-pyrazolo[4,3-c]pyridin-3-yl}-2-chlorophenyl)ethynyl]-2-chloro- | |
| | N-[(1R)-2-hydroxy-1-phenylethyl]benzamide | |
| 565 | 5-[(5-{5-[Amino(oxo)acetyl]-1-(3-thiomorpholin-4-ylpropyl)-4,5,6,7- | 696.22 |
| | tetrahydro-1H-pyrazolo[4,3-c]pyridin-3-yl}-2-chlorophenyl)ethynyl]-2-chloro- | |
| | N-[3-(methylamino)propyl]benzamide | |
| 566 | 2-(3-[4-Chloro-3-({4-chloro-3-[(ethylamino)methyl]phenyl}ethynyl)phenyl]-1- | 637.20 |
| | {3-[(3S)-3-methylmorpholin-4-yl]propyl}-1,4,6,7-tetrahydro-5H-pyrazolo[4,3- | |
| | c]pyridin-5-yl)-2-oxoacetamide | |
| 567 | 2-[3-(4-Chloro-3-{[4-chloro-3-(pyrrolidin-1- | 679.20 |
| | ylcarbonyl)phenyl]ethynyl}phenyl)-1-(3-thiomorpholin-4-ylpropyl)-1,4,6,7- | |
| | tetrahydro-5H-pyrazolo[4,3-c]pyridin-5-yl]-2-oxoacetamide | |
| 568 | 2-[3-(4-Chloro-3-{[4-chloro-3-(piperazin-1- | 694.21 |
| | ylcarbonyl)phenyl]ethynyl}phenyl)-1-(3-thiomorpholin-4-ylpropyl)-1,4,6,7- | |
| | tetrahydro-5H-pyrazolo[4,3-c]pyridin-5-yl]-2-oxoacetamide | |
| 569 | 2-[3-(4-Chloro-3-{[4-chloro-3-(1,4-diazepan-1- | 708.22 |
| | ylcarbonyl)phenyl]ethynyl}phenyl)-1-(3-thiomorpholin-4-ylpropyl)-1,4,6,7- | |
| | tetrahydro-5H-pyrazolo[4,3-c]pyridin-5-yl]-2-oxoacetamide | |
| 570 | 3-(4-Chloro-3-{[4-chloro-3-(pyrrolidin-1-ylcarbonyl)phenyl]ethynyl}phenyl)-1- | 651.20 |
| | (3-thiomorpholin-4-ylpropyl)-1,4,6,7-tetrahydro-5H-pyrazolo[4,3-c]pyridine- | |
| | 5-carboxamide | |
| 571 | 3-(4-Chloro-3-{[4-chloro-3-(piperazin-1-ylcarbonyl)phenyl]ethynyl}phenyl)-1- | 666.21 |
| | (3-thiomorpholin-4-ylpropyl)-1,4,6,7-tetrahydro-5H-pyrazolo[4,3-c]pyridine- | |
| | 5-carboxamide | |
| 572 | 3-(4-Chloro-3-{[4-chloro-3-(1,4-diazepan-1- | 680.23 |
| | ylcarbonyl)phenyl]ethynyl}phenyl)-1-(3-thiomorpholin-4-ylpropyl)-1,4,6,7- | |
| | tetrahydro-5H-pyrazolo[4,3-c]pyridine-5-carboxamide | |
| 573 | 2-[3-(4-Chloro-3-{[4-chloro-3-(pyrrolidin-1- | 677.23 |
| | ylcarbonyl)phenyl]ethynyl}phenyl)-1-{3-[(3S)-3-methylmorpholin-4- | |
| | yl]propyl}-1,4,6,7-tetrahydro-5H-pyrazolo[4,3-c]pyridin-5-yl]-2-oxoacetamide | |
| 574 | 2-[3-(4-Chloro-3-{[4-chloro-3-(piperazin-1- | 692.24 |
| | ylcarbonyl)phenyl]ethynyl}phenyl)-1-{3-[(3S)-3-methylmorpholin-4- | |
| | yl]propyl}-1,4,6,7-tetrahydro-5H-pyrazolo[4,3-c]pyridin-5-yl]-2-oxoacetamide | |
| 575 | 2-[3-(4-Chloro-3-{[4-chloro-3-(1,4-diazepan-1- | 706.20 |
| | ylcarbonyl)phenyl]ethynyl}phenyl)-1-{3-[(3S)-3-methylmorpholin-4- | |
| | yl]propyl}-1,4,6,7-tetrahydro-5H-pyrazolo[4,3-c]pyridin-5-yl]-2-oxoacetamide | |
| 576 | tert-Butyl (3-{[(5-{[5-(5-[amino(oxo)acetyl]-1-{3-[(3S)-3-methylmorpholin-4- | 794.30 |
| | yl]propyl}-4,5,6,7-tetrahydro-1H-pyrazolo[4,3-c]pyridin-3-yl)-2- | |
| | chlorophenyl]ethynyl}-2- | |
| | chlorophenyl)carbonyl]amino}propyl)methylcarbamate | |
| 577 | tert-Butyl (3-{[(5-{[5-(5-[amino(oxo)acetyl]-1-{3-[(3S)-3-methylmorpholin-4- | 780.20 |
| | yl]propyl}-4,5,6,7-tetrahydro-1H-pyrazolo[4,3-c]pyridin-3-yl)-2- | |
| | chlorophenyl]ethynyl}-2-chlorophenyl)carbonyl]amino}propyl)carbamate | |
| 578 | 5-[(5-{5-[Amino(oxo)acetyl]-1-(3-thiomorpholin-4-ylpropyl)-4,5,6,7- | 708.22 |
| | tetrahydro-1H-pyrazolo[4,3-c]pyridin-3-yl}-2-chlorophenyl)ethynyl]-2-chloro- | |
| | N-[(3R)-pyrrolidin-3-ylmethyl]benzamide | |
| 579 | 5-[(5-{5-[Amino(oxo)acetyl]-1-(3-thiomorpholin-4-ylpropyl)-4,5,6,7- | 724.20 |
| | tetrahydro-1H-pyrazolo[4,3-c]pyridin-3-yl}-2-chlorophenyl)ethynyl]-2-chloro- | |
| | N-(piperidin-3-ylmethyl)benzamide | |
| 580 | 5-[(5-{5-[Amino(oxo)acetyl]-1-(3-thiomorpholin-4-ylpropyl)-4,5,6,7- | 724.22 |
| | tetrahydro-1H-pyrazolo[4,3-c]pyridin-3-yl}-2-chlorophenyl)ethynyl]-2-chloro- | |
| | N-(morpholin-2-ylmethyl)benzamide | |
| 581 | 5-[(5-{5-[Amino(oxo)acetyl]-1-(3-thiomorpholin-4-ylpropyl)-4,5,6,7- | 739.20 |
| | tetrahydro-1H-pyrazolo[4,3-c]pyridin-3-yl}-2-chlorophenyl)ethynyl]-2-chloro- | |
| | N-(2-morpholin-4-ylethyl)benzamide | |
| 582 | 3-{4-Chloro-3-[(4-chloro-3-{[(3R)-pyrrolidin-3- | 680.23 |
| | ylmethyl]carbamoyl}phenyl)ethynyl]phenyl}-1-(3-thiomorpholin-4-ylpropyl)- | |
| | 1,4,6,7-tetrahydro-5H-pyrazolo[4,3-c]pyridine-5-carboxamide | |
| 583 | 3-[4-Chloro-3-({4-chloro-3-[(piperidin-3- | 694.30 |
| | ylmethyl)carbamoyl]phenyl}ethynyl)phenyl]-1-(3-thiomorpholin-4-ylpropyl)- | |
| | 1,4,6,7-tetrahydro-5H-pyrazolo[4,3-c]pyridine-5-carboxamide | |
| 584 | 3-[4-Chloro-3-({4-chloro-3-[(morpholin-2- | 696.22 |
| | ylmethyl)carbamoyl]phenyl}ethynyl)phenyl]-1-(3-thiomorpholin-4-ylpropyl)- | |
| | 1,4,6,7-tetrahydro-5H-pyrazolo[4,3-c]pyridine-5-carboxamide | |
| 585 | 3-[4-Chloro-3-({4-chloro-3-[(2-morpholin-4- | 710.24 |
| | ylethyl)carbamoyl]phenyl}ethynyl)phenyl]-1-(3-thiomorpholin-4-ylpropyl)- | |
| | 1,4,6,7-tetrahydro-5H-pyrazolo[4,3-c]pyridine-5-carboxamide | |
| 586 | 5-{[5-(5-[Amino(oxo)acetyl]-1-{3-[(3S)-3-methylmorpholin-4-yl]propyl}- | 706.26 |
| | 4,5,6,7-tetrahydro-1H-pyrazolo[4,3-c]pyridin-3-yl)-2-chlorophenyl]ethynyl}- | |
| | 2-chloro-N-[(3R)-pyrrolidin-3-ylmethyl]benzamide | |
| 587 | 5-{[5-(5-[Amino(oxo)acetyl]-1-{3-[(3S)-3-methylmorpholin-4-yl]propyl}- | 722.30 |
| | 4,5,6,7-tetrahydro-1H-pyrazolo[4,3-c]pyridin-3-yl)-2-chlorophenyl]ethynyl}- | |
| | 2-chloro-N-(piperidin-3-ylmethyl)benzamide | |
| 588 | 2-[3-(4-Chloro-3-{[4-chloro-3-(morpholin-4-ylmethyl)phenyl]ethynyl}phenyl)- | 679.25 |
| | 1-{3-[(3S)-3-methylmorpholin-4-yl]propyl}-1,4,6,7-tetrahydro-5H- | |
| | pyrazolo[4,3-c]pyridin-5-yl]-2-oxoacetamide | |
| 589 | 2-(3-[4-Chloro-3-({4-chloro-3- | 649.20 |
| | [(cyclopropylamino)methyl]phenyl}ethynyl)phenyl]-1-{3-[(3S)-3- | |
| | methylmorpholin-4-yl]propyl}-1,4,6,7-tetrahydro-5H-pyrazolo[4,3-c]pyridin- | |
| | 5-yl)-2-oxoacetamide | |
| 590 | 3-[4-Chloro-3-({4-chloro-3-[(2-morpholin-4- | 692.20 |
| | ylethyl)carbamoyl]phenyl}ethynyl)phenyl]-1-(3-piperidin-1-ylpropyl)-1,4,6,7- | |
| | tetrahydro-5H-pyrazolo[4,3-c]pyridine-5-carboxamide | |
| 591 | 5-[(5-{5-[Amino(oxo)acetyl]-1-(3-piperidin-1-ylpropyl)-4,5,6,7-tetrahydro-1H- | 720.20 |
| | pyrazolo[4,3-c]pyridin-3-yl}-2-chlorophenyl)ethynyl]-2-chloro-N-(2- | |
| | morpholin-4-ylethyl)benzamide | |
| 592 | 2-{3-[4-Chloro-3-({4-chloro-3-[(ethylamino)methyl]phenyl}ethynyl)phenyl]-1- | 621.24 |
| | (3-piperidin-1-ylpropyl)-1,4,6,7-tetrahydro-5H-pyrazolo[4,3-c]pyridin-5-yl}-2- | |
| | oxoacetamide | |
| 593 | 2-Chloro-5-({2-chloro-5-[5-(methylsulfonyl)-1-(3-piperidin-1-ylpropyl)- | 727.20 |
| | 4,5,6,7-tetrahydro-1H-pyrazolo[4,3-c]pyridin-3-yl]phenyl}ethynyl)-N-(2- | |
| | morpholin-4-ylethyl)benzamide | |
| 594 | N-[2-Chloro-5-({2-chloro-5-[5-(methylsulfonyl)-1-(3-piperidin-1-ylpropyl)- | 628.10 |
| | 4,5,6,7-tetrahydro-1H-pyrazolo[4,3-c]pyridin-3- | |
| | yl]phenyl}ethynyl)benzyl]ethanamine | |
| 595 | 2-{3-[4-Chloro-3-({4-chloro-3- | 661.20 |
| | [(cyclopentylamino)methyl]phenyl}ethynyl)phenyl]-1-(3-piperidin-1-ylpropyl)- | |
| | 1,4,6,7-tetrahydro-5H-pyrazolo[4,3-c]pyridin-5-yl}-2-oxoacetamide | |
| 596 | 3-[4-Chloro-3-({4-chloro-3- | 605.25 |
| | [(cyclopropylamino)methyl]phenyl}ethynyl)phenyl]-1-(3-piperidin-1-ylpropyl)- | |
| | 1,4,6,7-tetrahydro-5H-pyrazolo[4,3-c]pyridine-5-carboxamide | |
| 597 | N-[2-Chloro-5-({2-chloro-5-[5-(methylsulfonyl)-1-(3-piperidin-1-ylpropyl)- | 668.20 |
| | 4,5,6,7-tetrahydro-1H-pyrazolo[4,3-c]pyridin-3- | |
| | yl]phenyl}ethynyl)benzyl]cyclopentanamine | |
| 598 | 5-{[5-(5-[Amino(oxo)acetyl]-1-{3-[(3S)-3-methylmorpholin-4-yl]propyl}- | 694.10 |
| | 4,5,6,7-tetrahydro-1H-pyrazolo[4,3-c]pyridin-3-yl)-2-chlorophenyl]ethynyl}- | |
| | N-(azetidin-3-ylmethyl)-2-chlorobenzamide | |
| 599 | 3-[4-Chloro-3-({4-chloro-3- | 651.24 |
| | [(cyclopentylamino)methyl]phenyl}ethynyl)phenyl]-1-(3-thiomorpholin-4- | |
| | ylpropyl)-1,4,6,7-tetrahydro-5H-pyrazolo[4,3-c]pyridine-5-carboxamide | |
| 600 | 2-{3-[4-Chloro-3-({4-chloro-3- | 633.24 |
| | [(cyclopropylamino)methyl]phenyl}ethynyl)phenyl]-1-(3-piperidin-1-ylpropyl)- | |
| | 1,4,6,7-tetrahydro-5H-pyrazolo[4,3-c]pyridin-5-yl}-2-oxoacetamide | |
| 601 | 2-[3-(4-Chloro-3-{[4-chloro-3-(morpholin-4-ylmethyl)phenyl]ethynyl}phenyl)- | 663.20 |
| | 1-(3-piperidin-1-ylpropyl)-1,4,6,7-tetrahydro-5H-pyrazolo[4,3-c]pyridin-5-yl]- | |
| | 2-oxoacetamide | |
| 602 | 3-(4-Chloro-3-{[4-chloro-3-(morpholin-4-ylmethyl)phenyl]ethynyl}phenyl)-1- | 635.20 |
| | (3-piperidin-1-ylpropyl)-1,4,6,7-tetrahydro-5H-pyrazolo[4,3-c]pyridine-5- | |
| | carboxamide | |
| 603 | N-[2-Chloro-5-({2-chloro-5-[5-(methylsulfonyl)-1-(3-piperidin-1-ylpropyl)- | 640.10 |
| | 4,5,6,7-tetrahydro-1H-pyrazolo[4,3-c]pyridin-3- | |
| | yl]phenyl}ethynyl)benzyl]cyclopropanamine | |
| 604 | 3-(4-Chloro-3-{[4-chloro-3-(morpholin-4-ylmethyl)phenyl]ethynyl}phenyl)-5- | 670.10 |
| | (methylsulfonyl)-1-(3-piperidin-1-ylpropyl)-4,5,6,7-tetrahydro-1H- | |
| | pyrazolo[4,3-c]pyridine | |
| 605 | 3-[4-Chloro-3-({4-chloro-3- | 623.10 |
| | [(cyclopropylamino)methyl]phenyl}ethynyl)phenyl]-1-(3-thiomorpholin-4- | |
| | ylpropyl)-1,4,6,7-tetrahydro-5H-pyrazolo[4,3-c]pyridine-5-carboxamide | |
| 606 | 3-(4-Chloro-3-{[4-chloro-3-(morpholin-4-ylmethyl)phenyl]ethynyl}phenyl)-1- | 653.10 |
| | (3-thiomorpholin-4-ylpropyl)-1,4,6,7-tetrahydro-5H-pyrazolo[4,3-c]pyridine- | |
| | 5-carboxamide | |
| 607 | 3-[4-Chloro-3-({4-[(2-morpholin-4-ylethyl)carbamoyl]phenyl}ethynyl)phenyl]- | 676.20 |
| | 1-(3-thiomorpholin-4-ylpropyl)-1,4,6,7-tetrahydro-5H-pyrazolo[4,3- | |
| | c]pyridine-5-carboxamide | |
| 608 | 4-({2-Chloro-5-[5-(methylsulfonyl)-1-(3-piperidin-1-ylpropyl)-4,5,6,7- | 693.20 |
| | tetrahydro-1H-pyrazolo[4,3-c]pyridin-3-yl]phenyl}ethynyl)-N-(2-morpholin-4- | |
| | ylethyl)benzamide | |
| 609 | 3-{4-Chloro-3-[(4-{[(2S)-pyrrolidin-2- | 646.20 |
| | ylmethyl]carbamoyl}phenyl)ethynyl]phenyl}-1-(3-thiomorpholin-4-ylpropyl)- | |
| | 1,4,6,7-tetrahydro-5H-pyrazolo[4,3-c]pyridine-5-carboxamide | |
| 610 | 4-{[5-(5-[Amino(oxo)acetyl]-1-{3-[(3S)-3-methylmorpholin-4-yl]propyl}- | 672.20 |
| | 4,5,6,7-tetrahydro-1H-pyrazolo[4,3-c]pyridin-3-yl)-2-chlorophenyl]ethynyl}- | |
| | N-[(2S)-pyrrolidin-2-ylmethyl]benzamide | |
| 611 | 4-({2-Chloro-5-[5-(methylsulfonyl)-1-(3-piperidin-1-ylpropyl)-4,5,6,7- | 663.20 |
| | tetrahydro-1H-pyrazolo[4,3-c]pyridin-3-yl]phenyl}ethynyl)-N-[(2S)-pyrrolidin- | |
| | 2-ylmethyl]benzamide | |
| 612 | 3-{4-Chloro-3-[(4-{[3- | 634.27 |
| | (methylamino)propyl]carbamoyl}phenyl)ethynyl]phenyl}-1-(3-thiomorpholin- | |
| | 4-ylpropyl)-1,4,6,7-tetrahydro-5H-pyrazolo[4,3-c]pyridine-5-carboxamide | |
| 613 | 4-{[5-(5-[Amino(oxo)acetyl]-1-{3-[(3S)-3-methylmorpholin-4-yl]propyl}- | 660.20 |
| | 4,5,6,7-tetrahydro-1H-pyrazolo[4,3-c]pyridin-3-yl)-2-chlorophenyl]ethynyl}- | |
| | N-[3-(methylamino)propyl]benzamide | |
| 614 | 4-({2-Chloro-5-[5-(methylsulfonyl)-1-(3-piperidin-1-ylpropyl)-4,5,6,7- | 651.20 |
| | tetrahydro-1H-pyrazolo[4,3-c]pyridin-3-yl]phenyl}ethynyl)-N-[3- | |
| | (methylamino)propyl]benzamide | |
| 615 | 3-[4-Chloro-3-({4-chloro-3-[(dimethylamino)methyl]phenyl}ethynyl)phenyl]- | 611.20 |
| | 1-(3-thiomorpholin-4-ylpropyl)-1,4,6,7-tetrahydro-5H-pyrazolo[4,3- | |
| | c]pyridine-5-carboxamide | |
| 616 | 2-(3-[4-Chloro-3-({4-chloro-3- | 637.20 |
| | [(dimethylamino)methyl]phenyl}ethynyl)phenyl]-1-{3-[(3S)-3- | |
| | methylmorpholin-4-yl]propyl}-1,4,6,7-tetrahydro-5H-pyrazolo[4,3-c]pyridin- | |
| | 5-yl)-2-oxoacetamide | |
| 617 | 1-[2-Chloro-5-({2-chloro-5-[5-(methylsulfonyl)-1-(3-piperidin-1-ylpropyl)- | 628.20 |
| | 4,5,6,7-tetrahydro-1H-pyrazolo[4,3-c]pyridin-3-yl]phenyl}ethynyl)phenyl]- | |
| | N,N-dimethylmethanamine | |
| 618 | 2-[3-(4-Chloro-3-{[4-(hydroxymethyl)phenyl]ethynyl}phenyl)-1-{3-[(3S)-3- | 576.20 |
| | methylmorpholin-4-yl]propyl}-1,4,6,7-tetrahydro-5H-pyrazolo[4,3-c]pyridin- | |
| | 5-yl]-2-oxoacetamide | |
| 619 | 2-(3-{4-Chloro-3-[(4-chloro-3-{[(1- | 651.20 |
| | methylethyl)amino]methyl}phenyl)ethynyl]phenyl}-1-{3-[(3S)-3- | |
| | methylmorpholin-4-yl]propyl}-1,4,6,7-tetrahydro-5H-pyrazolo[4,3-c]pyridin- | |
| | 5-yl)-2-oxoacetamide | |
| 620 | 2-(3-{4-Chloro-3-[(4-{[(4-chlorobenzyl)amino]methyl}phenyl)ethynyl]phenyl}- | 699.20 |
| | 1-{3-[(3R)-3-methylmorpholin-4-yl]propyl}-1,4,6,7-tetrahydro-5H- | |
| | pyrazolo[4,3-c]pyridin-5-yl)-2-oxoacetamide | |
| 621 | 2-(3-{4-Chloro-3-[(4-{[(4-chlorobenzyl)amino]methyl}phenyl)ethynyl]phenyl}- | 700.20 |
| | 1-[3-(3-methylmorpholin-4-yl)propyl]-1,4,6,7-tetrahydro-5H-pyrazolo[4,3- | |
| | c]pyridin-5-yl)-2-oxoacetamide | |

### Biological Testing:

Recombinant human cathepsin S (CatS) was expressed in the baculovirus system and purified in one step with a thiopropyl-sepharose column. 10-L yielded ˜700 mg of CatS and N-terminal sequencing confirmed identity. The assay is run in 150 mM sodium acetate pH 5.0 containing 1.5 mM DTT and 150 mM NaCl. The substrate for the assay is: Z-Valine-Valine-Arginine-AMC (catalog #1-1540, Bachem). The Kₘ for the substrate is around 5 μM but the presence of substrate inhibition makes kinetic analysis difficult. With 10 μM substrate the assay rate is linear over the range of 1-8 ng CatS in 100 μL reaction. Using 2 ng/well of CatS, the production of product is linear and yields-7-fold signal after 20 min with only 20% loss of substrate. Measurements are taken every min for 20 min. The rate is calculated from the slope of the increase in fluorescence and the percent inhibition is calculated from this.

Results for the compounds tested in this assay are presented in Tables 2, 3, and 4 as an average of results obtained. Compounds were tested in free base, hydrochloride salt, trifluoroacetic acid salt, citric acid salt, or formic acid salt forms.

TABLE 2

| | CatS IC₅₀ |
|---|---|
| EX. | (μM) |
| 1 | 0.19 |
| 2 | 0.35 |
| 3 | 0.27 |
| 4 | 0.75 |
| 5 | 0.29 |
| 6 | 0.95 |
| 7 | 0.25 |
| 8 | 0.19 |
| 9 | 0.16 |
| 10 | 0.42 |
| 11 | 2.60 |
| 12 | 0.79 |
| 13 | 0.32 |
| 14 | 20.0 |
| 15 | 0.15 |
| 16 | 1.15 |
| 17 | 1.90 |
| 18 | 0.18 |
| 19 | 0.42 |
| 20 | 0.54 |
| 21 | 1.32 |
| 22 | 1.85 |
| 23 | 1.44 |
| 24 | 0.56 |
| 25 | 0.70 |
| 26 | 0.72 |
| 27 | 1.01 |
| 28 | 0.20 |
| 29 | 0.28 |
| 30 | 0.43 |
| 31 | 0.44 |
| 32 | 0.49 |
| 33 | 0.75 |
| 34 | 0.87 |
| 35 | 1.05 |
| 36 | 1.45 |
| 37 | 0.12 |
| 38 | 0.33 |
| 39 | 0.61 |
| 40 | 0.41 |
| 41 | 3.95 |
| 42 | 9.75 |
| 43 | 5.45 |
| 44 | 7.85 |
| 45 | 0.92 |
| 46 | 7.95 |
| 47 | 10.6 |
| 48 | 6.75 |
| 49 | 19.5 |
| 50 | 6.80 |
| 51 | 8.25 |
| 52 | 13.5 |
| 53 | 15.5 |
| 54 | 7.05 |
| 55 | 1.07 |
| 56 | 0.91 |
| 57 | 0.23 |
| 58 | 0.53 |
| 59 | 0.66 |
| 60 | 0.88 |
| 61 | 0.20 |
| 62 | 1.23 |
| 63 | 1.40 |
| 64 | 5.40 |
| 65 | 1.07 |
| 66 | 0.25 |
| 67 | 3.05 |
| 68 | 2.85 |
| 69 | 0.53 |
| 70 | 0.27 |
| 71 | 0.17 |
| 72 | 0.49 |
| 73 | 0.35 |
| 74 | 0.43 |
| 75 | 0.35 |
| 76 | 0.24 |
| 77 | 0.64 |
| 78 | 1.40 |
| 79 | 1.50 |
| 80 | 9.93 |
| 81 | 5.47 |
| 82 | 6.10 |
| 83 | 7.10 |
| 84 | 8.10 |
| 85 | 8.93 |
| 86 | 13.0 |
| 87 | 11.4 |
| 88 | 0.52 |
| 89 | 0.29 |
| 90 | 0.42 |
| 91 | 0.94 |
| 92 | 1.04 |
| 93 | 1.05 |
| 94 | 1.10 |
| 95 | 1.15 |
| 96 | 1.20 |
| 97 | 1.31 |
| 98 | 1.41 |
| 99 | 2.45 |
| 100 | 2.50 |
| 101 | 2.06 |
| 102 | 0.84 |
| 103 | 0.31 |
| 104 | 0.93 |
| 105 | 1.43 |
| 106 | 4.42 |
| 107 | 0.50 |
| 108 | 0.66 |
| 109 | 2.27 |
| 110 | 3.03 |
| 111 | 2.04 |
| 112 | 4.92 |
| 113 | 3.58 |
| 114 | 1.52 |
| 115 | 1.69 |
| 116 | 0.04 |
| 117 | 0.04 |
| 118 | 0.13 |
| 119 | 0.06 |
| 120 | 0.03 |
| 121 | 0.15 |
| 122 | 0.26 |
| 123 | 0.01 |
| 124 | 0.15 |
| 125 | 0.08 |
| 126 | 0.01 |
| 127 | 0.02 |
| 128 | 0.11 |
| 129 | 0.12 |
| 130 | 0.08 |
| 131 | 0.16 |
| 132 | 0.39 |
| 133 | 0.29 |
| 134 | 0.18 |
| 135 | 0.06 |
| 136 | 0.05 |
| 137 | 0.14 |
| 138 | 0.35 |
| 139 | 0.23 |
| 140 | 0.25 |
| 141 | 0.31 |
| 142 | 0.28 |
| 143 | 0.22 |
| 144 | 0.06 |
| 145 | 0.09 |
| 146 | 0.06 |
| 147 | 0.41 |
| 148 | 0.08 |
| 149 | 0.11 |

TABLE 3

| | CatS IC₅₀ |
|---|---|
| EX. | (μM) |
| 150 | 0.08 |
| 151 | 0.09 |
| 152 | 0.11 |
| 153 | 0.18 |
| 154 | 0.26 |
| 155 | 0.79 |
| 156 | 0.05 |
| 157 | 0.04 |
| 158 | 0.05 |
| 159 | 0.06 |
| 160 | 0.06 |
| 161 | 0.07 |
| 162 | 0.09 |
| 163 | 0.13 |
| 164 | 0.73 |
| 165 | 0.83 |
| 166 | 2.52 |
| 167 | 0.03 |
| 168 | 0.03 |
| 169 | 0.05 |
| 170 | 0.05 |
| 171 | 0.20 |
| 172 | 0.03 |
| 173 | 0.12 |
| 174 | 0.27 |
| 175 | 0.19 |
| 176 | 0.23 |
| 177 | 0.01 |
| 178 | 0.01 |
| 179 | 0.02 |
| 180 | 0.02 |
| 181 | 0.06 |
| 182 | 0.08 |
| 183 | 0.08 |
| 184 | 0.32 |
| 185 | 0.52 |
| 186 | 0.69 |
| 187 | 0.15 |
| 188 | 0.55 |
| 189 | 0.14 |
| 190 | 1.11 |
| 191 | 0.63 |
| 192 | 0.08 |
| 193 | 0.05 |
| 194 | 0.02 |
| 195 | 0.13 |
| 196 | 0.65 |
| 197 | 0.20 |
| 198 | 0.07 |
| 199 | 0.06 |
| 200 | 0.09 |
| 201 | 0.19 |
| 202 | 0.39 |
| 203 | 0.57 |
| 204 | 0.12 |
| 205 | 0.27 |
| 206 | 0.21 |
| 207 | 2.86 |
| 208 | 3.13 |
| 209 | 0.10 |
| 210 | 0.19 |
| 211 | 0.11 |
| 212 | 0.08 |
| 213 | 0.02 |
| 214 | 0.04 |
| 215 | 0.03 |
| 216 | 0.07 |
| 217 | 0.02 |
| 218 | 0.03 |
| 219 | 0.04 |
| 220 | 0.03 |
| 221 | 0.02 |
| 222 | 0.02 |
| 223 | 0.02 |
| 224 | 0.02 |
| 225 | 0.01 |
| 226 | 0.03 |
| 227 | 0.04 |
| 228 | 0.09 |
| 229 | 0.09 |
| 230 | 0.01 |
| 231 | 2.69 |
| 232 | 1.53 |
| 233 | 7.92 |
| 234 | 0.28 |
| 235 | 0.33 |
| 236 | 7.13 |
| 237 | 0.01 |
| 238 | 0.003 |
| 239 | 0.61 |
| 240 | 0.01 |
| 241 | 0.03 |
| 242 | 0.03 |
| 243 | 0.04 |
| 244 | 0.06 |
| 245 | 0.03 |
| 246 | 0.01 |
| 247 | 0.07 |
| 248 | 0.02 |
| 249 | 0.05 |
| 250 | 0.08 |
| 251 | 0.04 |
| 252 | 0.11 |
| 253 | 0.06 |
| 254 | 0.24 |
| 255 | 0.07 |
| 256 | 0.05 |
| 257 | 0.07 |
| 258 | 0.08 |
| 259 | 0.08 |
| 260 | 0.13 |
| 261 | 0.13 |
| 262 | 0.12 |
| 263 | 0.13 |
| 264 | 0.37 |
| 265 | 0.14 |
| 266 | 0.35 |
| 267 | 0.01 |
| 268 | 0.02 |
| 269 | 0.01 |
| 270 | 0.02 |
| 271 | 0.02 |
| 272 | 0.03 |
| 273 | 0.04 |
| 274 | 0.04 |
| 275 | 0.05 |
| 276 | 0.18 |
| 277 | 0.20 |
| 278 | 0.11 |
| 279 | 0.11 |
| 280 | 0.04 |
| 281 | 0.12 |
| 282 | 0.15 |
| 283 | 0.10 |
| 284 | 0.20 |
| 285 | 0.02 |
| 286 | 0.03 |
| 287 | 0.05 |
| 288 | 0.12 |
| 289 | 0.01 |
| 290 | 0.21 |
| 291 | 0.03 |
| 292 | 0.02 |
| 293 | 0.05 |
| 294 | 0.02 |
| 295 | 0.03 |

TABLE 4

| | CatS IC₅₀ |
|---|---|
| EX. | (μM) |
| 296 | 15.50 |
| 297 | 1.60 |
| 298 | 0.05 |
| 299 | 4.80 |
| 300 | 9.20 |
| 301 | 7.05 |
| 302 | 11.50 |
| 303 | >20 |
| 304 | 17.00 |
| 305 | 3.95 |
| 306 | 3.22 |
| 307 | 4.65 |
| 308 | 5.65 |
| 309 | 0.32 |
| 310 | 2.12 |
| 311 | 1.35 |
| 312 | 5.62 |
| 313 | 1.96 |
| 314 | 3.66 |
| 315 | 1.36 |
| 316 | 0.58 |
| 317 | 2.71 |
| 318 | 1.15 |
| 319 | 0.13 |
| 320 | 0.09 |
| 321 | 4.65 |
| 322 | 0.73 |
| 323 | 0.75 |
| 324 | 1.34 |
| 325 | 1.45 |
| 326 | 1.37 |
| 327 | 0.76 |
| 328 | 0.02 |
| 329 | 0.03 |
| 330 | 0.09 |
| 331 | 0.17 |
| 332 | 0.19 |
| 333 | 0.18 |
| 334 | 0.03 |
| 335 | 0.06 |
| 336 | 0.15 |
| 337 | 0.07 |
| 338 | 0.02 |
| 339 | 0.23 |
| 340 | 0.39 |
| 341 | 0.83 |
| 342 | 0.31 |
| 343 | 0.04 |
| 344 | 0.06 |
| 345 | 0.11 |
| 346 | 0.02 |
| 347 | 0.61 |
| 348 | 0.10 |
| 349 | 0.55 |
| 350 | 0.04 |
| 351 | 0.12 |
| 352 | 0.02 |
| 353 | 0.15 |
| 354 | 0.36 |
| 355 | 0.11 |
| 356 | 0.02 |
| 357 | 0.21 |
| 358 | 0.10 |
| 359 | 0.08 |
| 360 | 0.17 |
| 361 | 0.17 |
| 362 | 0.05 |
| 363 | 0.09 |
| 364 | 0.17 |
| 365 | 0.28 |
| 366 | 0.68 |
| 367 | 0.09 |
| 368 | 0.33 |
| 369 | 0.41 |
| 370 | 0.72 |
| 371 | 0.38 |
| 372 | 0.06 |
| 373 | 0.17 |
| 374 | 0.16 |
| 375 | 0.41 |
| 376 | 1.01 |
| 377 | 0.47 |
| 378 | 0.08 |
| 379 | 0.29 |
| 380 | 0.06 |
| 381 | 0.10 |
| 382 | 0.11 |
| 383 | 0.12 |
| 384 | 2.79 |
| 385 | 2.72 |
| 386 | 0.77 |
| 387 | 0.13 |
| 388 | 0.06 |
| 389 | 0.23 |
| 390 | 0.15 |
| 391 | 0.26 |
| 392 | 0.18 |
| 393 | 0.22 |
| 394 | 3.81 |
| 395 | 0.02 |
| 396 | 0.23 |
| 397 | 0.51 |
| 398 | 0.38 |
| 399 | 0.11 |
| 400 | 0.19 |
| 401 | 0.11 |
| 402 | 0.15 |
| 403 | 8.52 |
| 404 | 0.37 |
| 405 | 0.29 |
| 406 | 0.46 |
| 407 | 0.02 |
| 408 | 0.28 |
| 409 | 0.02 |
| 410 | 0.04 |
| 411 | 0.13 |
| 412 | 0.16 |
| 413 | 0.10 |
| 414 | 0.03 |
| 415 | 0.09 |
| 416 | 0.07 |
| 417 | 0.02 |
| 418 | 1.03 |
| 419 | 0.18 |
| 420 | 0.10 |
| 421 | 0.19 |
| 422 | 0.17 |
| 423 | 0.27 |
| 424 | 0.20 |
| 425 | 0.07 |
| 426 | 0.39 |
| 427 | 1.99 |
| 428 | 3.75 |
| 429 | 0.02 |
| 430 | 0.04 |
| 431 | 0.17 |
| 432 | 0.23 |
| 433 | 0.24 |
| 434 | 0.20 |
| 435 | 0.08 |
| 436 | 0.28 |
| 437 | 0.57 |
| 438 | 0.72 |
| 439 | 0.32 |
| 440 | 0.39 |
| 441 | 3.06 |
| 442 | 0.30 |
| 443 | 0.69 |
| 444 | 0.21 |
| 445 | 0.20 |
| 446 | 0.07 |
| 447 | 0.07 |
| 448 | 0.01 |
| 449 | 0.10 |
| 450 | 0.07 |
| 451 | 0.10 |
| 452 | 0.10 |
| 453 | 0.14 |
| 454 | 0.04 |
| 455 | 0.04 |
| 456 | 0.12 |
| 457 | 0.09 |
| 458 | 0.06 |
| 459 | 0.02 |
| 460 | 0.17 |
| 461 | 0.03 |
| 462 | 0.05 |
| 463 | 0.11 |
| 464 | 0.63 |
| 465 | 0.15 |
| 466 | 0.05 |
| 467 | 0.26 |
| 468 | 0.19 |
| 469 | 0.53 |
| 470 | 0.05 |
| 471 | 7.42 |
| 472 | 4.51 |
| 473 | 0.12 |
| 474 | 0.43 |
| 475 | 0.18 |
| 476 | 0.12 |
| 477 | 0.10 |
| 478 | 0.04 |
| 479 | 0.02 |
| 480 | 0.10 |
| 481 | 3.52 |
| 482 | 0.18 |
| 483 | 0.14 |
| 484 | 0.18 |
| 485 | 0.05 |
| 486 | 0.03 |
| 487 | 0.06 |
| 488 | 0.14 |
| 489 | 0.48 |
| 490 | 0.07 |
| 491 | 0.12 |
| 492 | 0.22 |
| 493 | 0.30 |
| 494 | 0.31 |
| 495 | 0.77 |
| 496 | 0.07 |
| 497 | 1.63 |
| 498 | 4.56 |
| 499 | 0.16 |
| 500 | 0.58 |
| 501 | 0.75 |
| 502 | 0.11 |
| 503 | 0.66 |
| 504 | 0.28 |
| 505 | 0.63 |
| 506 | 0.52 |
| 507 | 0.57 |
| 508 | 0.11 |
| 509 | 0.50 |
| 510 | 0.45 |
| 511 | 1.61 |
| 512 | 0.22 |
| 513 | 0.23 |
| 514 | 0.14 |
| 515 | 0.25 |
| 516 | 0.19 |
| 517 | 0.19 |
| 518 | 0.39 |
| 519 | 0.40 |
| 520 | 0.44 |
| 521 | 0.37 |
| 522 | 0.33 |
| 523 | 0.53 |
| 524 | 0.53 |
| 525 | 0.35 |
| 526 | 0.37 |
| 527 | 0.30 |
| 528 | 0.58 |
| 529 | 0.12 |
| 530 | 1.33 |
| 531 | 0.64 |
| 532 | 0.09 |
| 533 | 0.10 |
| 534 | 0.09 |
| 535 | 0.64 |
| 536 | 0.20 |
| 537 | 0.28 |
| 538 | 0.11 |
| 539 | 0.21 |
| 540 | 0.14 |
| 541 | 0.96 |
| 542 | 0.18 |
| 543 | 0.13 |
| 544 | 0.13 |
| 545 | 0.27 |
| 546 | 0.30 |
| 547 | 0.12 |
| 548 | 0.12 |
| 549 | 1.01 |
| 550 | 0.10 |
| 551 | 0.10 |
| 552 | 0.50 |
| 553 | 0.45 |
| 554 | 0.02 |
| 555 | 0.02 |
| 556 | 0.03 |
| 557 | 0.03 |
| 558 | 0.03 |
| 559 | 0.10 |
| 560 | 0.02 |
| 561 | 0.03 |
| 562 | 0.03 |
| 563 | 0.03 |
| 564 | 0.03 |
| 565 | 0.02 |
| 566 | 0.02 |
| 567 | 0.16 |
| 568 | 0.14 |
| 569 | 0.15 |
| 570 | 0.26 |
| 571 | 0.13 |
| 572 | 0.17 |
| 573 | 0.19 |
| 574 | 0.14 |
| 575 | 0.17 |
| 576 | 0.05 |
| 577 | 0.02 |
| 578 | 0.02 |
| 579 | 0.02 |
| 580 | 0.03 |
| 581 | 0.03 |
| 582 | 0.04 |
| 583 | 0.03 |
| 584 | 0.04 |
| 585 | 0.04 |
| 586 | 0.02 |
| 587 | 0.02 |
| 588 | 0.04 |
| 589 | 0.03 |
| 590 | 0.07 |
| 591 | 0.03 |
| 592 | 0.02 |
| 593 | 0.06 |
| 594 | 0.03 |
| 595 | 0.01 |
| 596 | 0.03 |
| 597 | 0.04 |
| 598 | 0.03 |
| 599 | 0.03 |
| 600 | 0.03 |
| 601 | 0.03 |
| 602 | 0.04 |
| 603 | 0.05 |
| 604 | 0.08 |
| 605 | 0.04 |
| 606 | 0.04 |
| 607 | 0.05 |
| 608 | 0.07 |
| 609 | 0.07 |
| 610 | 0.03 |
| 611 | 0.10 |
| 612 | 0.08 |
| 613 | 0.04 |
| 614 | 0.06 |
| 615 | 0.03 |
| 616 | 0.012 |
| 617 | 0.04 |
| 618 | 0.02 |
| 619 | 0.02 |
| 620 | 0.15 |
| 621 | 0.13 |

While the invention has been illustrated by reference to examples, it is understood that the invention is intended not to be limited to the foregoing detailed description.

What is claimed is:

1. A compound of Formula (I): 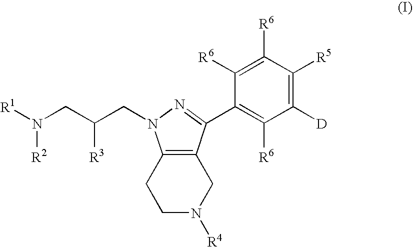
wherein:
R¹ and R² taken together with the nitrogen to which they are attached form a saturated monocyclic heterocycloalkyl group, each group optionally containing one additional heteroatom ring member that is O, S, or NR^{a}, and each group unsubstituted or substituted with one, two, or three R^{b} substituents;
where R^{a} is H, C₁₋₄alkyl, —COC₁₋₄alkyl, —CO(phenyl), or —CO₂C₁₋₄alkyl, or a monocyclic cycloalkyl ring, phenyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R^{b} substituent is independently:
i) OH, C₁₋₄alkyl, —C₁₋₄alkyl-OH, CF₃, —NR^{c}R^{d}, halo, —OC₁₋₄alkyl, —COC₁₋₄alkyl, —CO₂C₁₋₄alkyl, —CO₂H, or —CONR^{e}R^{f};
ii) a monocyclic heterocycloalkyl group unsubstituted or substituted with C₁₋₄alkyl, —COC₁₋₄alkyl, —CO₂C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group or monocyclic heteroaryl group, each group unsubstituted or substituted with C₁₋₄alkyl, OH, —OC₁₋₄alkyl, —NR^{c}R^{d}, or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo;
vi) two R^{b} substituents form a methylene or ethylene bridge; or
vii) two R^{b} substituents on adjacent carbons taken together with the carbons to which they are attached form a saturated monocyclic cycloalkyl group or saturated monocyclic heterocycloalkyl group, each group unsubstituted or substituted with C₁₋₄alkyl, OH, —OC₁₋₄alkyl, —NR^{c}R^{d}, or halo;
where R^{c} is H or C₁₋₄alkyl;
R^{d} is H, C₁₋₄alkyl, —COC₁₋₄alkyl, —COC₁₋₄alkyl-OH, —CO₂C₁₋₄alkyl, —CONR^{x}R^{y}, or —SO₂C₁₋₄alkyl;
where R^{x} and R^{y} are each independently H or C₁₋₄alkyl; and
R^{e} and R^{f} are each independently H or C₁₋₄alkyl;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, or —NR^{t}R^{u}; —COCF₃; —CO-(monocyclic heteroaryl); —CO—(C-linked monocyclic heterocycloalkyl); —CO-(phenyl); —SO₂C₁₋₄alkyl; —SO₂CF₃; —SO₂NR^{t}R^{u}; —CONR^{t}R^{u}; —COCO₂C₁₋₄alkyl; or —COCONR^{t}R^{u};
where R^{t} and R^{u} are each independently H, C₁₋₄alkyl, or —COC₁₋₄alkyl; or R^{t} and R^{u} taken together with the nitrogen to which they are attached form a monocyclic heterocycloalkyl ring;
R⁵ is halo or CF₃;
each R⁶ is independently H or F;
D is —C≡C—R⁷, —CH═CH—R³, —(CH₂)₂₋₃—R³, or —(CH₂)₃₋₅—R⁹;
where R⁷ is:
I) a C₁₋₄alkyl group unsubstituted or substituted with OH, —OC₁₋₄alkyl, —NR^{g}R^{h} phenyl, or phenoxy, each phenyl or phenoxy being unsubstituted or substituted with C₁₋₄alkyl, OH, —OC₁₋₄alkyl, halo, or CF₃;
where R^{g} and R^{h} are each independently H, C₁₋₄alkyl, —COC₁₋₄alkyl, —COphenyl, —CO₂C₁₋₄alkyl, —SO₂C₁₋₄alkyl, or —SO₂-phenyl; or R^{g} and R^{h} taken together with the nitrogen to which they are attached form a monocyclic heterocycloalkyl group; or
II) a monocyclic cycloalkyl group, phenyl group, or monocyclic heteroaryl group, each group unsubstituted or substituted with one or two R^{k} substituents;
where R⁸ is a phenyl group or monocyclic heteroaryl group, each group unsubstituted or substituted with one or two R^{k} substituents;
where R⁹ is OH or —NRⁿR^{o};
where Rⁿ is H or C₁₋₄alkyl; and
R^{o} is H, C₁₋₄alkyl, monocyclic cycloalkyl, —COC₁₋₄alkyl, —COphenyl, —CO₂C₁₋₄alkyl, —SO₂C₁₋₄alkyl, —SO₂-phenyl, —SO₂-benzyl, or —SO₂NR^{p}R^{g}, each phenyl or benzyl group being unsubstituted or substituted with one or two R^{k} substituents;
where R^{p} and R^{g} are each independently H or C₁₋₄alkyl;
or Rⁿ and R^{o} taken together with the nitrogen to which they are attached form a monocyclic saturated heterocycloalkyl ring unsubstituted or substituted with C₁₋₄alkyl, OH, —OC₁₋₄alkyl, halo, or CF₃;
where each R^{k} substituent in D is independently:
a) a C₁₋₄alkyl group unsubstituted or substituted with OH, —OC₁₋₄alkyl, —OC₁₋₄alkyl-OH, halo, —CO₂C₁₋₄alkyl, CO₂H, CN, —NR^{r}R^{s}, —N(R^{r})CO-phenyl, —N(R^{r})SO₂C₁₋₄alkyl, —N(R^{r}) SO₂-phenyl, —SO₂C₁₋₄alkyl, phenyl, or phenoxy;
b) a substituent of formula 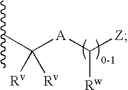
where each R^{v} is independently H or C₁₋₄alkyl, or both R^{v} substituents together form a carbonyl;
R^{w} is H, C₁₋₄alkyl, —CH₂OH, or —CO₂C₁₋₄alkyl;
A is O or NR^{aa};
where R^{aa} is H or C₁₋₄alkyl; and
Z is a phenyl group, benzyl group, cycloalkyl group, heterocycloalkyl group, heteroaryl group, or —CH₂— (heteroaryl) group, each group unsubstituted or substituted with one or two substituents independently selected from the group consisting of C₁₋₄alkyl, CF₃, halo, OH, —OC₁₋₄alkyl, —OCF₃, —OCHF₂, NR^{dd}R^{ee}, —CO₂C₁₋₄alkyl, —SC₁₋₄alkyl, and —SO₂C₁₋₄alkyl;
where R^{dd} and R^{ee} are each independently H or C₁₋₄alkyl;
c) two adjacent R^{k} substituents taken together with the carbons to which they are attached form a fused phenyl ring, monocyclic heteroaryl ring, monocyclic heterocycloalkyl ring, or monocyclic cycloalkyl ring, each fused ring unsubstituted or substituted with: C₁₋₄alkyl, —C₁₋₄alkyl-CF₃, —C₁₋₄alkyl-OH, —C₁₋₄alkyl-CO₂C₁₋₄alkyl, CF₃, C₂₋₄alkenyl, halo, OH, —OC₁₋₄alkyl, —COC₁₋₄alkyl, —COCF₃, —CO₂C₁₋₄alkyl, —CO₂H, —CONR^{ff}R^{gg}, or —SO₂C₁₋₄alkyl; or with a cycloalkyl group, —CH₂-(cycloalkyl) group, or benzyl group, each group unsubstituted or substituted with C₁₋₄alkyl, OH, —OC₁₋₄alkyl, halo, or CF₃;
where R^{ff} and R^{gg} are each independently H or C₁₋₄alkyl, or R^{ff} and R^{gg} taken together with the nitrogen to which they are attached form a monocyclic heterocycloalkyl ring, unsubstituted or substituted with C₁₋₄alkyl or OH; or
d) OH; —OC₁₋₄alkyl; halo; CF₃; —CHO; —CO₂C₁₋₄alkyl; CO₂H; CN; —NO₂; —CONR^{r}R^{s}, —NR^{r}R^{s}; —N(R^{r})-phenyl; —N(R^{r})-benzyl; —N(R^{r})-phenethyl; —N(R^{r})COC₁₋₄-alkyl; —N (R^{r}) CO-phenyl; —N(R^{r})SO₂C₁₋₄alkyl; —N (R^{r})SO₂-phenyl; —SO₂C₁₋₄alkyl; phenoxy;
or a heteroaryl group; where each phenyl, benzyl, phenethyl, phenoxy, or heteroaryl group is unsubstituted or substituted with C₁₋₄alkyl, OH, —OC₁₋₄alkyl, halo, or CF₃;
where R^{r} is H, C₁₋₄alkyl, C₂₋₄alkyl-OH; and
R^{s} is H, C₁₋₄alkyl, —C₁₋₄alkyl-CF₃, —C₁₋₄alkyl-CN, —C₂₋₄alkyl-OH, —C₂₋₄alkyl-NR^{bb}R^{cc}, —C₁₋₄alkylCO₂C₁₋₄alkyl, —C₁₋₄alkylCO₂H, C₃₋₄alkenyl, —COC₁₋₄alkyl, or —CO₂C₁₋₄alkyl;
where R^{bb} is H or C₁₋₄alkyl; and
R^{cc} is H, C₁₋₄alkyl, —COC₁₋₄alkyl, or —CO₂C₁₋₄alkyl;
or R^{bb} and R^{cc} taken together with the nitrogen to which they are attached from a monocyclic heterocycloalkyl ring;
or R^{r} and R^{s} taken together with the nitrogen to which they are attached form a heterocycloalkyl group unsubstituted or substituted with C₁₋₄alkyl, OH, —OC₁₋₄alkyl, halo, CF₃, or a monocyclic heterocycloalkyl ring unsubstituted or substituted with OH;
or a pharmaceutically acceptable salt, prodrug, or metabolite thereof.

2. A compound as defined in claim 1, wherein —NR¹R² is a structure of Formula (II): 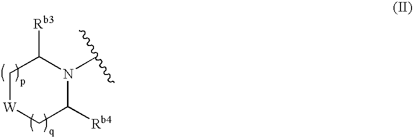
wherein:
W is NR^{a}, O, S, or C(R^{b1})(R^{b2});
where R^{a} is H or C₁₋₄alkyl;
R^{b1} is H, OH, or C₁₋₄alkyl; and
R^{b2} is H; C₁₋₄alkyl; a monocyclic heterocycloalkyl group unsubstituted or substituted with C₁₋₄alkyl, OH, —OC₁₋₄alkyl, NR^{c}R^{d}, or halo; or a monocyclic heterocycloalkyl group fused with a phenyl or pyridyl group, the resulting fused bicyclic group being unsubstituted or substituted with C₁₋₄alkyl, OH, —OC₁₋₄alkyl, NR^{c}R^{d}, or halo;
one of R^{b3} and R^{b4} is H and the other is H or C₁₋₄alkyl;
p is 0, 1, or 2; and
q is 0, 1, 2, or 3;
with the proviso that when W is NR^{a}, O, or S, then p and q are each greater than or equal to 1.

3. A compound as defined in claim 1, wherein —NR¹R² is a structure of Formula (III): 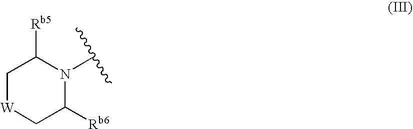
wherein W is O or S; and R^{b5} and R^{b6} are each independently H or C₁₋₄alkyl.

4. A compound as defined in claim 1, wherein R¹ and R² taken together with the nitrogen to which they are attached form azetidine, pyrrolidine, piperidine, piperazine substituted with R^{a}, morpholine, or thiomorpholine, each unsubstituted or substituted with one, two, or three R^{b} substituents.

5. A compound as defined in claim 1, wherein R¹ and R² taken together with the nitrogen to which they are attached form piperidine, piperazine substituted with R^{a}, or morpholine, each unsubstituted or substituted with one, two, or three R^{b} substituents.

6. A compound as defined in claim 1, wherein R¹ and R² taken together with the nitrogen to which they are attached form 1,1-dioxo-1λ⁶-thiomorpholine, thiomorpholine 1-oxide, piperazinone substituted with R^{a}, [1,4]oxazepane, each unsubstituted or substituted with one, two, or three R^{b} substituents; or 2,5-diaza-bicyclo[2.2.1]heptane substituted with R^{a}, 2-oxa-5-aza-bicyclo[2.2.1]heptane, 2-oxa-6-aza-spiro[3.3]heptane, or hexahydro-furo[3,4-c]pyrrole, each of the latter four groups unsubstituted or substituted with one R substituent.

7. A compound as defined in claim 1, wherein R^{a} is H, methyl, isopropyl, acetyl, or tert-butoxycarbonyl.

8. A compound as defined in claim 1, wherein R^{a} is phenyl, 2-hydroxyphenyl, 3-hydroxyphenyl, 4-hydroxyphenyl, benzoyl, pyridyl, 1-hydroxy-pyridyl, or cyclobutyl.

9. A compound as defined in claim 1, wherein each R^{b} substituent is independently OH, methyl, CF₃, methoxycarbonyl, dimethylamino, acetamido, tert-butoxycarbamoyl, fluoro, or methoxy.

10. A compound as defined in claim 1, wherein each R^{b} substituent is independently carbamoyl, amino, ethoxycarbonyl, carboxy, hydroxymethyl, 2-hydroxyacetylamino, methanesulfonylamino, or tert-butyl; or two R substituents on the same carbon taken together with the carbon to which they are attached form a dioxolane ring.

11. A compound as defined in claim 1, wherein each R^{b} substituent is independently pyrrolidinyl, 2-oxo-pyrrolidinyl, or piperidinyl, each optionally substituted.

12. A compound as defined in claim 1, wherein each R^{b} substituent is independently 2-oxo-piperidinyl, morpholinyl, 1-tert-butoxycarbonyl-piperidin-4-yl, 1-methyl-piperidin-4-yl, or 1-acetyl-piperidin-4-yl.

13. A compound as defined in claim 1, wherein each R^{b} substituent is independently pyrrolidin-1-yl, 2-oxo-pyrrolidin-1-yl, or 5-dimethylamino-1-methyl-1,3-dihydro-imidazo[4,5-b]pyridin-2-onyl, or two R substituents on the same carbon taken together with the carbon to which they are attached form 2-oxo-pyrrolidin-3-yl.

14. A compound as defined in claim 1, wherein each R^{b} substituent is independently phenyl or pyridyl, each optionally substituted.

15. A compound as defined in claim 1, wherein R³ is H or OH.

16. A compound as defined in claim 1, wherein R⁴ is —SO₂CH₃, —CONH₂, or —COCONH₂.

17. A compound as defined in claim 1, wherein R⁴ is dimethylaminooxalyl, acetyl, dimethylsulfamoyl, methylcarbamoyl, dimethylcarbamoyl, 2-aminoacetyl, 2-acetoxyacetyl, 2-acetylamino-acetyl, tetrahydrofuran-2-carbonyl, or morpholine-4-carbonyl.

18. A compound as defined in claim 1, wherein R⁴ is —SO₂CH₃.

19. A compound as defined in claim 1, wherein R⁵ is chloro or CF₃.

20. A compound as defined in claim 1, wherein R⁵ is chloro.

21. A compound as defined in claim 1, wherein each R⁶ is H.

22. A compound as defined in claim 1, wherein D is —C≡C—R⁷, and R⁷ is benzyl, phenethyl, phenpropyl, hydroxymethyl, 2-hydroxyethyl, 3-hydroxypropyl, butyl, phenoxymethyl, 2-methylpropyl, diethylaminomethyl, (1,1-dioxo-1λ⁶-thiomorpholin-4-yl)-methyl, benzamidomethyl, or (benzenesulfonamido)methyl.

23. A compound as defined in claim 1, wherein R⁷ is cyclopentyl, cyclohexyl, phenyl, thiophenyl, or pyridyl, each unsubstituted or substituted with one or two R^{k} substituents.

24. A compound as defined in claim 1, wherein R⁷ is phenyl, unsubstituted or substituted with two R^{k} substituents.

25. A compound as defined in claim 1, wherein each R^{k} substituent in D is independently a methyl group or ethyl group unsubstituted or substituted with OH, methoxy, fluoro, —CO₂CH₃, CO₂H, CN, amino, tert-butoxycarbamoyl, methylsulfonamido, acetamido, pyrrolidinyl, or piperidinyl.

26. A compound as defined in claim 1, wherein each R^{k} substituent in D is a methyl group substituted with NR^{r}R^{s}.

27. A compound as defined in claim 1, wherein each R^{k} substituent in D is methylaminomethyl, dimethylaminomethyl, diethylaminomethyl, isobutylaminomethyl, tert-butoxycarbonylamino-methyl, (2-hydroxyethyl)aminomethyl, (3-hydroxypropyl)aminomethyl, (methoxycarbonylmethyl-amino)-methyl, (carboxymethyl-amino)-methyl, (2,2,2-trifluoroethyl-amino)-methyl, allylamino-methyl, (2-hydroxy-2-methyl-propylamino)-methyl, ethylaminomethyl, propylaminomethyl, [bis-(2-hydroxy-ethyl)-amino]-methyl, 3-hydroxy-propoxymethyl, phenylsulfonylamino-methyl, or benzoylamino-methyl.

28. A compound as defined in claim 1, wherein each R^{k} substituent in D is 3,4-dihydro-1H-isoquinolin-2-ylmethyl, 1,3-dihydro-isoindol-2-ylmethyl, 4-(2-oxo-pyrrolidin-1-yl)-piperidin-1-ylmethyl, or 4-(4-hydroxy-2-oxo-pyrrolidin-1-yl)-piperidin-1-ylmethyl, morpholin-4-ylmethyl.

29. A compound as defined in claim 1, wherein each R^{k} substituent in D is independently OH, methoxy, chloro, bromo, fluoro, CF₃, CO₂H, CN, amino, dimethylamino, acetylamino, methylsulfonamido, or methylsulfonyl.

30. A compound as defined in claim 1, wherein each R^{k} substituent in D is phenoxy, 4-iodo-phenoxy, benzylamino, cyanomethyl-amino, benzimidazol-2-yl, phenethyl-amino, 3-(tert-butoxycarbonyl-methyl-amino)-propylcarbamoyl, 3-methylamino-propylcarbamoyl, pyrrolidine-1-carbonyl, 3-hydroxy-pyrrolidine-1-carbonyl, piperazine-1-carbonyl, [1,4]diazepane-1-carbonyl, 3-hydroxy-propylcarbamoyl, or 2-morpholin-4-yl-ethylcarbamoyl.

31. A compound as defined in claim 1, wherein R^{k} is a substituent of formula

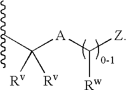

32. A compound as defined in claim 31, wherein R^{k} is phenethylamino-methyl, cyclopropylamino-methyl, cyclobutylamino-methyl, cyclopentylamino-methyl, cyclohexylamino-methyl, cyclopropylmethylamino-methyl, benzylamino-methyl, (4-chloro-benzylamino)-methyl, (4-methanesulfonyl-benzylamino)-methyl, (2-chloro-benzylamino)-methyl, (3-chloro-benzylamino)-methyl, (2-fluoro-benzylamino)-methyl, (3-fluoro-benzylamino)-methyl, (4-fluoro-benzylamino)-methyl, (3,4-dichloro-benzylamino)-methyl, (2-methoxy-benzylamino)-methyl, (3-methoxy-benzylamino)-methyl, (4-methoxy-benzylamino)-methyl, (2-methyl-benzylamino)-methyl, (3-methyl-benzylamino)-methyl, (4-methyl-benzylamino)-methyl, (4-dimethylamino-benzylamino)-methyl, (4-isopropoxy-benzylamino)-methyl, (4-difluoromethoxy-benzylamino)-methyl, (4-amino-benzylamino)-methyl, (benzyl)-methyl-amino)-methyl, [(4-chloro-benzyl)-methyl-amino]-methyl, (1-phenyl-ethylamino)-methyl, phenylaminomethyl, [(pyridin-2-ylmethyl)-amino]-methyl, [(pyridin-3-ylmethyl)-amino]-methyl, [(pyridin-4-ylmethyl)-amino]-methyl, (2-hydroxy-1-phenyl-ethylamino)-methyl, [(methoxycarbonyl-phenyl-methyl)-amino]-methyl, [(thiophen-2-ylmethyl)-amino]-methyl, [(thiophen-3-ylmethyl)-amino]-methyl, (2-thiophen-2-yl-ethylamino)-methyl, [(3-methyl-thiophen-2-ylmethyl)-amino]-methyl, [(furan-2-ylmethyl)-amino]-methyl, [(2-trifluoromethyl-furan-3-ylmethyl)-amino]-methyl, (1,2,3,4-tetrahydro-naphthalen-1-ylamino)-methyl, indan-1-ylaminomethyl, (2-hydroxy-indan-1-ylamino)-methyl, [(thiazol-2-ylmethyl)-amino]-methyl, [(1-methyl-1H-imidazol-2-ylmethyl)-amino]-methyl, [(tetrahydro-furan-2-ylmethyl)-amino]-methyl, or [(tetrahydro-pyran-4-ylmethyl)-amino]-methyl.

33. A compound as defined in claim 31, wherein R^{k} is (pyridin-2-ylmethyl)-carbamoyl, (pyridin-3-ylmethyl)-carbamoyl, (pyridin-4-ylmethyl)-carbamoyl, benzyl-carbamoyl, (4-chlorobenzyl)-carbamoyl, (pyrrolidin-2-ylmethyl)-carbamoyl, (pyrrolidin-3-ylmethyl)-carbamoyl, 2-hydroxy-1-phenyl-ethylcarbamoyl, (morpholin-2-ylmethyl)-carbamoyl, (piperidin-3-ylmethyl)-carbamoyl, or (azetidin-3-ylmethyl)-carbamoyl.

34. A compound as defined in claim 31, wherein R^{k} is pyridin-2-ylmethoxymethyl, pyridin-3-ylmethoxymethyl, pyridin-4-ylmethoxymethyl, piperidin-4-ylmethoxymethyl, morpholin-2-ylmethoxymethyl, pyrrolidin-3-yloxymethyl, or 1-tert-butoxycarbonyl-pyrrolidin-3-yloxymethyl.

35. A compound as defined in claim 1, wherein two adjacent R^{k} substituents taken together with the ring to which they are attached form a bicyclic fused ring system selected from the group consisting of indole, tetrahydroisoquinoline, 3,4-dihydro-2H-isoquinolin-1-one, 2,3,4,5-tetrahydro-1H-benzo[d]azepine, 2,3,4,5-tetrahydro-1H-benzo[c]azepine, 2,3-dihydro-1H-isoindole, benzimidazole, imidazole, 1H-pyrrolo[2,3-b]pyridine, and 5,6,7,8-tetrahydro-[1,2,4]triazolo[4,3-a]pyrazine, each fused ring system optionally substituted.

36. A compound as defined in claim 35, wherein the fused ring system is substituted with methyl, isopropyl, isobutyl, 2,2,2-trifluoroethyl, hydroxymethyl, ethoxycarbonylmethyl, allyl, acetyl, —COCF₃, tert-butoxycarbonyl, methoxycarbonyl, carboxy, carbamoyl, methylcarbamoyl, dimethylcarbamoyl, pyrrolidine-1-carbonyl, piperidine-1-carbonyl, 4-methyl-piperazine-1-carbonyl, or morpholine-4-carbonyl.

37. A compound as defined in claim 1, wherein R^{r} is H or methyl.

38. A compound as defined in claim 1, wherein R^{s} is H, methyl, acetyl, or tert-butoxycarbonyl.

39. A compound as defined in claim 1, wherein R^{r} and R^{s} taken together with the nitrogen to which they are attached form azetidinyl, pyrrolidinyl, or piperidinyl, each unsubstituted or substituted with methyl, OH, methoxy, fluoro, or CF₃.

40. A compound as defined in claim 1, wherein R⁷ is 1H-indol-5-yl, 4-cyanomethyl-phenyl, 3-cyanomethyl-phenyl, 4-hydroxymethyl-phenyl, 3-hydroxymethyl-phenyl, 4-hydroxy-phenyl, 4-(3-carboxy-propyl)-phenyl, 4-(2-carboxy-ethyl)-phenyl, 4-(methoxycarbonyl)methyl-phenyl, 3-(methoxycarbonyl)methyl-phenyl, thiophen-2-yl, 3,4-dichloro-phenyl, 4-(4-iodo-phenoxy)-phenyl, 4-carboxymethyl-phenyl, 3-carboxymethyl-phenyl, 4-phenoxy-phenyl, 4-bromo-phenyl, 4-carboxy-phenyl, pyridin-4-yl, pyridin-3-yl, pyridin-2-yl, thiophen-3-yl, 2-methoxy-phenyl, 3-chlorophenyl, 2-chlorophenyl, 3-hydroxyphenyl, 4-chlorophenyl, 4-methylphenyl, 4-trifluoromethylphenyl, 4-fluorophenyl, 4-methoxyphenyl, 2,4-difluorophenyl, 2-trifluoromethylphenyl, 2-methylphenyl, 3-trifluoromethylphenyl, 4-amino-phenyl, phenyl, 4-(tert-butoxcarbamoyl)methyl-phenyl, benzyl, phenethyl, phenpropyl, hydroxymethyl, 2-hydroxyethyl, 3-hydroxypropyl, butyl, cyclohexyl, (diethylamino)methyl, (1,1-dioxo-1 A⁶-thiomorpholin-4-yl)methyl, 2-methylpropyl, phenoxymethyl, (benzamido)methyl, (benzenesulfonamido)methyl, 4-acetamido-phenyl, 4-aminomethyl-phenyl, 4-(methanesulfonamido)methyl-phenyl, 4-(benzenesulfonamido)methyl-phenyl, 4-(acetamido)methyl-phenyl, 4-(benzamido)methyl-phenyl, 4-(benzylamino)methyl-phenyl, 4-(4-methyl-benzylamino)methyl-phenyl, 4-(4-chloro-benzylamino)methyl-phenyl, 4-[benzyl(methyl)amino]methyl-phenyl, 4-pyrrolidin-1-ylmethyl-phenyl, 4-piperidin-1-ylmethyl-phenyl, or 1,2,3,4-tetrahydro-isoquinolin-1-yl.

41. A compound as defined in claim 1, wherein D is —CH═CH—R³ or —(CH₂)₂₋₃—R³, and R³ is phenyl, unsubstituted or substituted with one or two R^{k} substituents.

42. A compound as defined in claim 1, wherein R⁸ is 1H-indol-5-yl, phenyl, 4-phenoxyphenyl, 3-hydroxyphenyl, 4-chlorophenyl, 4-methoxyphenyl, 2,4-difluorophenyl, 2-methylphenyl, or 4-hydroxymethyl-phenyl.

43. A compound as defined in claim 1, wherein D is —(CH₂)₃₋₅—R⁹, and R⁹ is OH.

44. A compound as defined in claim 1, wherein R⁹ is NRⁿR^{o}.

45. A compound as defined in claim 1, wherein R⁹ is dimethylamino, cyclopentylamino, acetamido, or methanesulfonamido.

46. A compound as defined in claim 1, wherein R⁹ is benzamido, benzene-sulfonamido, or benzylsulfonamido, each unsubstituted or substituted with one or two R^{k} substituents.

47. A compound as defined in claim 1, wherein R⁹ is pyrrolidine, piperidine, morpholine, piperazine, or azepine, each unsubstituted or substituted with methyl, OH, fluoro, or CF₃.

48. A compound as defined in claim 1, wherein R⁹ is OH, benzamido, methanesulfonamido, benzene-sulfonamido, benzylsulfonamido, 3,4-dichlorobenzene-sulfonamido, 4-chlorobenzene-sulfonamido, 4-methylbenzenesulfonamido, 4-methoxybenzene-sulfonamido, N,N-dimethyl-sulfamoylurea, acetamido, 2-carboxybenzenesulfonamido, 2-nitrobenzene-sulfonamido, 3-chlorobenzene-sulfonamido, 3-methoxybenzene-sulfonamido, 2-methylbenzene-sulfonamido, 2-chlorobenzenesulfonamido, 3-nitrobenzenesulfonamido, 3-methylbenzenesulfonamido, 3-cyanobenzenesulfonamido, 3-methanesulfonyl-benzenesulfonamido, 2-methanesulfonyl-benzenesulfonamido, pyrrolidin-1-yl, piperidin-1-yl, 3-methyl-pipieridin-1-yl, 4,4-difluoro-piperidin-1-yl, morpholin-4-yl, 4-methyl-piperazin-1-yl, azepan-1-yl, or cyclopentylamino.

49. A compound as defined in claim 1, wherein compounds of Formula (I) are selected from compounds of the following Formula (IV): 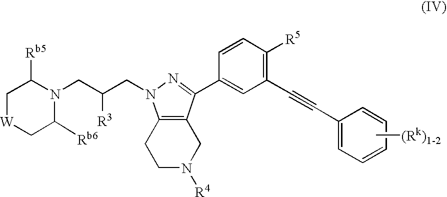
wherein:
W is O or S;
R^{b5} and R^{b6} are each independently H or C₁₋₄alkyl;
R³ is H or OH;
R⁴ is —SO₂CH₃, —CONH₂, or —COCONH₂;
R⁵ is chloro; and
each R^{k} substituent is independently:
a) a methyl group or ethyl group, each group unsubstituted or substituted with OH, —OC₁₋₄alkyl, halo, —CO₂C₁₋₄alkyl, CO₂H, CN, —NR^{r}R^{s}, —N(R^{r})COphenyl, —N(R^{r})SO₂C₁₋₄alkyl, —N(R^{r}) SO₂-phenyl, or —SO₂C₁₋₄alkyl;
where R^{r} is H, C₁₋₄alkyl, C₂₋₄alkyl-OH; and
R^{s} is H, C₁₋₄alkyl, C₁₋₄alkyl-CF₃, C₁₋₄alkyl-CN, C₂₋₄alkyl-OH, C₂₋₄alkyl-NR^{bb}R^{cc}, —C₁₋₄alkylCO₂C₁₋₄alkyl, —C₁₋₄alkylCO₂H, C₃₋₄alkenyl, —COC₁₋₄alkyl, or —CO₂C₁₋₄alkyl;
where R^{bb} is H or C₁₋₄alkyl; and
R^{cc} is H, C₁₋₄alkyl, —COC₁₋₄alkyl, or —CO₂C₁₋₄alkyl;
or R^{bb} and R^{cc} taken together with the nitrogen to which they are attached from a monocyclic heterocycloalkyl ring;
or R^{r} and R^{s} taken together with the nitrogen to which they are attached form a heterocycloalkyl group unsubstituted or substituted with C₁₋₄alkyl, OH, —OC₁₋₄alkyl, halo, CF₃, or a monocyclic heterocycloalkyl ring unsubstituted or substituted with OH;
b) a substituent of formula 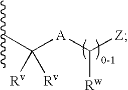
where each R^{v} is independently H or C₁₋₄alkyl, or both R^{v} substituents together form a carbonyl;
R^{w} is H, C₁₋₄alkyl, —CH₂OH, or —CO₂C₁₋₄alkyl;
A is O or NR^{aa};
where R^{aa} is H or C₁₋₄alkyl; and
Z is a phenyl group, benzyl group, cycloalkyl group, heterocycloalkyl group, heteroaryl group, or —CH₂— (heteroaryl) group, each group unsubstituted or substituted with one or two substituents independently selected from the group consisting of C₁₋₄alkyl, CF₃, halo, OH, —OC₁₋₄alkyl, —OCF₃, —OCHF₂, NR^{dd}R^{ee}, —CO₂C₁₋₄alkyl, —SC₁₋₄alkyl, and —SO₂C₁₋₄alkyl;
where R^{dd} and R^{ee} are each independently H or C₁₋₄alkyl; or
c) two adjacent R^{k} substituents taken together with the carbons to which they are attached form a fused phenyl ring, monocyclic heteroaryl ring, monocyclic heterocycloalkyl ring, or monocyclic cycloalkyl ring, each fused ring unsubstituted or substituted with: C₁₋₄alkyl, —C₁₋₄alkyl-CF₃, —C₁₋₄alkyl-OH, —C₁₋₄alkyl-CO₂C₁₋₄alkyl, CF₃, C₂₋₄alkenyl, halo, OH, —OC₁₋₄alkyl, —COC₁₋₄alkyl, —COCF₃, —CO₂C₁₋₄alkyl, —CO₂H, CONR^{ff}R^{gg}, or —SO₂C₁₋₄alkyl; or a with cycloalkyl group, —CH₂— (cycloalkyl) group, or benzyl group, each group unsubstituted or substituted with C₁₋₄alkyl, OH, —OC₁₋₄alkyl, halo, or CF₃;
where R^{ff} and R^{gg} are each independently H or C₁₋₄alkyl, or R^{ff} and R^{gg} taken together with the nitrogen to which they are attached form a monocyclic heterocycloalkyl ring, unsubstituted or substituted with C₁₋₄alkyl or OH;
and pharmaceutically acceptable salts, prodrugs, and metabolites thereof.

50. A compound selected from the group consisting of:
3-[4-Chloro-3-(1H-indol-5-ylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[2-(1H-indol-5-yl)-ethyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
4-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-butyric acid;
3-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-propionic acid;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
3-(4-Chloro-3-thiophen-2-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3,4-dichloro-phenylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[4-(4-iodo-phenoxy)-phenylethynyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
3-[4-Chloro-3-(4-phenoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[3-(4-Bromo-phenylethynyl)-4-chloro-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzoic acid;
3-(4-Chloro-3-pyridin-4-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-3-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-2-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-thiophen-3-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
3-[4-Chloro-3-(4-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-p-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(4-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2,4-difluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-o-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzaldehyde;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenylamine;
3-(4-Chloro-3-phenylethynyl-phenyl)-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-carbamic acid tert-butyl ester;
3-[4-Chloro-3-(3-phenyl-prop-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-phenyl-but-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(5-phenyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-yn-1-ol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-but-3-yn-1-ol;
5-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-pent-4-yn-1-ol;
3-(4-Chloro-3-hex-1-ynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-cyclohexylethynyl-phenyl)-5-methanesulfonyl-1-(3-morpholin-4-yl-propyl)-4,5,6,7-tetrahydro-1H-pyrazolo[4,3-c]pyridine;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-diethyl-amine;
3-{4-Chloro-3-[3-(1,1-dioxo-1 A⁶-thiomorpholin-4-yl)-prop-1-ynyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(4-methyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-phenoxy-prop-1-ynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzenesulfonamide;
3-{4-Chloro-3-[2-(4-phenoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenol;
3-{4-Chloro-3-[2-(4-chloro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-{4-Chloro-3-[2-(4-methoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-{4-Chloro-3-[2-(2,4-difluoro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(2-o-tolyl-ethyl)-phenyl]-5-methanesulfonyl-1-(3-morpholin-4-yl-propyl)-4,5,6,7-tetrahydro-1H-pyrazolo[4,3-c]pyridine;
[4-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenyl]-methanol;
3-(4-Chloro-3-phenethyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-phenyl-propyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetamide;
3-(4-Chloro-3-Z-styryl-phenyl)-5-methanesulfonyl-1-(3-morpholin-4-yl-propyl)-4,5,6,7-tetrahydro-1H-pyrazolo[4,3-c]pyridine;
3-(4-Chloro-3-E-styryl-phenyl)-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzylamine;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methanesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-acetamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzamide;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-(4-methyl-benzyl)-amine;
(4-Chloro-benzyl)-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methyl-amine;
3-[4-Chloro-3-(4-pyrrolidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-piperidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-methanesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-C-phenyl-methanesulfonamide;
3,4-Dichloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
4-Chloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methyl-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methoxy-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-N,N-dimethyl-sulfamoylurea;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-acetamide;
2-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propylsulfamoyl}-benzoic acid;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-nitro-benzenesulfonamide;
3-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-4-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methoxy-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methyl-benzenesulfonamide;
2-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-nitro-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-cyano-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methanesulfonyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methanesulfonyl-benzenesulfonamide;
1-[1-(3-{3-[4-Chloro-3-(3-pyrrolidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-piperidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(3-methyl-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[3-(4,4-difluoro-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(3-morpholin-4-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(4-methyl-piperazin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(3-Azepan-1-yl-propyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-cyclopentylamino-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-pyrrolidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-piperidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[4-(3-methyl-piperidin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[4-(4-methyl-piperazin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(4-morpholin-4-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Azepan-1-yl-butyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-cyclopentylamino-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
8-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-2,8-diaza-spiro[4.5]decan-1-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid amide;
3-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-1,3-dihydro-imidazo[4,5-b]pyridin-2-one;
[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
1-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-3-morpholin-4-yl-propan-2-ol;
2-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-cyclopentanone;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-morpholin-4-yl-propan-2-ol;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperidin-1-yl-propan-2-ol;
3-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-,3-dihydro-imidazo[4,5-b]pyridin-2-one;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid amide;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-pyrrolidin-1-yl-propan-2-ol;
[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
4-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperazine-1-carboxylic acid tert-butyl ester;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperazin-1-yl-propan-2-ol;
1-(4-Amino-piperidin-1-yl)-3-{3-[4-chloro-3-(4-chloro-phenylethynyl)-phenyl]-5-methanesulfonyl-4,5,6,7-tetrahydro-pyrazolo[4,3-c]pyridin-1-yl}-propan-2-ol;
1-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-3-piperidin-1-yl-propan-2-ol;
1-(3-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-propyl)-piperidine-4-carboxylic acid amide;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-oxo-acetic acid methyl ester;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-pyridin-2-yl-methanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-furan-2-yl-methanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2,2,2-trifluoro-ethanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-fluoro-ethanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-(tetrahydro-furan-2-yl)-methanone;
Acetic acid 2-[3-[4-chloro-3-(4-chloro-phenylethynyl)-phenyl]-1-(3-morpholin-4-yl-propyl)-1,4,6,7-tetrahydro-pyrazolo[4,3-c]pyridin-5-yl]-2-oxo-ethyl ester;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-hydroxy-ethanone;
3-[4-Chloro-3-(4-chloro-phenylethynyl)-phenyl]-1-(3-morpholin-4-yl-propyl)-1,4,6,7-tetrahydro-pyrazolo[4,3-c]pyridine-5-carboxylic acid amide;
3-{4-Chloro-3-[2-(4-chloro-phenyl)-ethyl]-phenyl}-1-(3-morpholin-4-yl-propyl)-1,4,6,7-tetrahydro-pyrazolo[4,3-c]pyridine-5-carboxylic acid amide;
1-{1-[3-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-pyrrolidin-2-one;
1-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-3-morpholin-4-yl-propan-2-ol;
8-[3-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-2-hydroxy-propyl]-2,8-diaza-spiro[4.5]decan-1-one; and
1-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-3-morpholin-4-yl-propan-2-ol;
and pharmaceutically acceptable salts thereof.

51. A compound selected from the group consisting of:
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoacetamide;
3-(4-Chloro-3-[4-({[(4-chlorophenyl)methyl]aminomethyl)phenyl]ethynyl]phenyl)-1-(3-morpholin-4-ylpropyl)-1,4,6,7-tetrahydro-5H-pyrazolo[4,3-c]pyridine-5-carboxamide;
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-N,N-dimethyl-2-oxoacetamide;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-sulfonamide;
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoethanamine;
3-(4-Chloro-3-{[4-({[(4-chlorophenyl)methyl]amino]methyl)phenyl]ethynyl]phenyl)-N-methyl-1-(3-morpholin-4-ylpropyl)-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-pyridin-2-ylpiperazin-1-yl)propyl]-1,4,6,7-tetrahydro-5H-pyrazolo[4,3-c]pyridine-5-carboxamide;
2-(3-{4-Chloro-3-[(4-{[(4-chlorobenzyl)amino]methyl-phenyl)ethynyl]phenyl}-1-{3-[(3S,5S)-3,5-dimethylmorpholin-4-yl]propyl}-1,4,6,7-tetrahydro-5H-pyrazolo[4,3-c]pyridin-5-yl)-2-oxoacetamide;
2-[3-(4-Chloro-3-[4-(([(4-chlorophenyl)methyl]amino]methyl)phenyl]ethynyl]phenyl)-{3-[(3S)-3-methylmorpholin-4-yl]propyl}-1,4,6,7-tetrahydro-5H-pyrazolo[4,3-c]pyridin-5-yl]-2-oxoacetamide;
2-{3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridin-5-yl}-2-oxoacetamide;
3-(4-Chloro-3-{[4-({[(4-chlorophenyl)methyl]amino]methyl)phenylethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-[(pyridin-3-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide; 3-{4-Chloro-3-[(4-[(pyridin-3-ylmethyl)amino]carbonyl]phenyl)ethynyl]phenyl}-1-(3-thiomorpholin-4-ylpropyl)-1,4,6,7-tetrahydro-5H-pyrazolo[4,3-c]pyridine-5-carboxamide; 3-{4-Chloro-3-[(4-{[(piperidin-4-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[4-(2-oxopyrrolidin-1-yl)piperidin-1-yl]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
1-[4-({2-Chloro-5-[1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-({4-chloro-3-[(ethylamino)methyl]phenyl}ethynyl)phenyl]-1-(3-piperidin-1-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(2-morpholin-4-ylethyl)benzamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl]-N-(2-morpholin-4-ylethyl)benzamide;
3-{4-Chloro-3-[(4-{[(4-chlorobenzyl)amino]methyl(phenyl)ethynyl]phenyl}-1-{3-[(3aR,6aS)-tetrahydro-1H-furo[3,4-c]pyrrol-5(3H)-yl]propyl}-1,4,6,7-tetrahydro-5H-pyrazolo[4,3-c]pyridine-5-carboxamide;
3-(4-Chloro-3-{[3-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoethanol;
3-(4-Chloro-3-{[4-({[(4-methylphenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(1-methylethyl)oxy]phenyl}methyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-({4-[({[4-(dimethylamino)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(difluoromethyl)oxy]phenyl]methyl)amino]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
1-{4-[(2-Chloro-5-{5-(methylsulfonyl)-1-[3-(4-pyridin-2-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperidin-1-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-[4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)benzaldehyde;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydro-2H-pyran-4-ylmethyl)methanamine;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(tetrahydro-2H-pyran-4-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
Methyl N-{[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycine;
2-(1,1-Dimethylethyl) 3-methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-methyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N,N-dimethyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
Methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
Methyl (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
2-(1,1-Dimethylethyl) 3-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
2-(1,1-Dimethylethyl) 1-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-1,2(1H)-dicarboxylate;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-1-carboxylate;
(2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
Methyl (2R)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3,4-dichlorophenyl)methyl]methanamine;
(1S,2R)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl]phenyl)ethynyl]phenyl}methyl)amino]-2,3-dihydro-1H-inden-1-ol;
(1R,2S)-1-[({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl methyl)amino]-2,3-dihydro-1H-inden-2-ol;
(1R)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1S)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1R)—N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(2S)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)amino]-2-phenylethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
Methyl N-{[5-({5-[5-(aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl}ethynyl)-2-chlorophenyl]methyl}glycinate;
N-{[5-({5-[5-(Aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl]ethynyl)-2-chlorophenyl]methyl]glycine;
3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridine-5-carboxylic acid amide;
3-{4-Chloro-3-[(4-chloro-3-{[(tetrahydrofuran-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
3-{4-Chloro-3-[(4-chloro-3-{[(phenylmethyl)amino]methyl(phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-methyl-1,2,3,4-tetrahydroisoquinoline;
N-[1-(3-{3-[4-Chloro-3-(1,2,3,4-tetrahydroisoquinolin-7-ylethynyl)phenyl]-5-(methylsulfonyl)-4,5,6,7-tetrahydro-1H-pyrazolo[4,3-c]pyridin-1-yl}propyl)piperidin-4-yl]acetamide;
7-(2-{2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethyl)-1,2,3,4-tetrahydroisoquinoline;
7-[2-(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethyl]-1,2,3,4-tetrahydroisoquinoline;
1,1-Dimethylethyl 1′-{(2S)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
1,1-Dimethylethyl 1′-[(2S)-3-[3-[4-chloro-3-(4-methylpent-1-yn-1-yl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(cyclohexylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl}-2-hydroxypropyl]-4,4′-bipiperidine-1-carboxylate;
1,1-Dimethylethyl 1′-[(2S)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-(2S)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]-2-hydroxypropyl-4,4′-bipiperidine-1-carboxylate;
1,1-Dimethylethyl 1′-{(2S)-3-[5-(aminocarbonyl)-3-{4-chloro-3-[(4-chlorophenyl)ethynyl]phenyl}-4,5,6,7-tetrahydro-1H-pyrazolo[4,3-c]pyridin-1-yl]-2-hydroxypropyl}-4,4′-bipiperidine-1-carboxylate;
1-[(2S)-3-(4,4′-Bipiperidin-1-yl)-2-hydroxypropyl]-3-{4-chloro-3-[(4-chlorophenyl)ethynyl]phenyl}-1,4,6,7-tetrahydro-5H-pyrazolo[4,3-c]pyridine-5-carboxamide;
(2S)-1-(4,4′-Bipiperidin-1-yl)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl}propan-2-ol;
(2S)-1-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(2S)-1-(1′-Acetyl-4,4′-bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
1,1-Dimethylethyl 1′-{(2S)-3-[5-(aminocarbonyl)-3-{4-chloro-3-[2-(4-chlorophenyl)ethyl]phenyl}-4,5,6,7-tetrahydro-1H-pyrazolo[4,3-c]pyridin-1-yl]-2-hydroxypropyl}-4,4′-bipiperidine-1-carboxylate;
2-[3-(4-Chloro-3-{2-[4-({[(4-chlorophenyl)methyl]amino{methyl)phenyl]ethyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
3-(4-Chloro-3-{2-[4-({[(4-chlorophenyl)methyl]amino]methyl)phenyl]ethyl]phenyl)-1-{3-[(3S)-3-methylmorpholin-4-yl]propyl}-1,4,6,7-tetrahydro-5H-pyrazolo[4,3-c]pyridine-5-carboxamide;
1,1-Dimethylethyl 1′-{(2S)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
(2S)-1-(4,4′-Bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-(1′-Acetyl-4,4′-bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-[3-{4-Chloro-3-[2-(4-chlorophenyl)ethyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(S)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(S)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(S)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(2-{3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
2-{3-(4-Chloro-3-{4-[(4-chloro-benzylamino)-methyl]-phenylethynyl}-phenyl)-1-[(2R)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
(S)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
2-{3-[4-Chloro-3-(4-chloro-3-{[((2S)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
2-{3-[4-Chloro-3-(4-chloro-3-{[((2R)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-cyclopentylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-propylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[3-(3-{[Bis-(2-hydroxy-ethyl)-amino]-methyl}-4-chloro-phenylethynyl)-4-chloro-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-5-(5-{5-Aminooxalyl-1-[3-(3-methyl-morpholin-4-yl)-propyl]-4,5,6,7-tetrahydro-1H-pyrazolo[4,3-c]pyridin-3-yl}-2-chloro-phenylethynyl)-2-chloro-N-(3-methylamino-propyl)-benzamide;
(S)-2-{3-{4-Chloro-3-[4-chloro-3-(3-hydroxy-propoxymethyl)-phenylethynyl]-phenyl}-1-[3-(3-methyl-morpholin-4-yl)-propyl]-1,4,6,7-tetrahydro-pyrazolo[4,3-c]pyridin-5-yl}-2-oxo-acetamide;
(S)-2-[3-[4-Chloro-3-[4-chloro-3-(pyrrolidin-3-yloxymethyl)-phenylethynyl]-phenyl]-1-[3-(3-methyl-morpholin-4-yl)-propyl]-1,4,6,7-tetrahydro-pyrazolo[4,3-c]pyridin-5-yl]-2-oxo-acetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-(1-methylethyl)-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
2-(3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
2-(3-{4-Chloro-3-[(4-chloro-3-{[(phenylmethyl)amino]methyl-phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-(2-{2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine; 2-(3-{4-Chloro-3-[(4-chloro-3-{[(pyridin-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-(phenylmethyl)methanamine;
3-{4-Chloro-3-[(4-chloro-3-[(phenylmethyl)amino]methyl(phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-chloro-3-[(pyridin-2-ylmethyl)amino]methyl(phenyl)ethynyl]phenyl}-1-(3-thiomorpholin-4-ylpropyl)-1,4,6,7-tetrahydro-5H-pyrazolo[4,3-c]pyridine-5-carboxamide;
2-({[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]methyl]amino)ethanol;
Methyl N-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)methyl]glycinate; and
2-(3-{4-Chloro-3-[(4-chloro-3-{[(2-hydroxyethyl)amino]methylphenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
and pharmaceutically acceptable salts thereof.

52. A compound selected from the group consisting of:
3-(2-{3-[5-(Methylsulfonyl)-1-(3-morpholin-4-ylpropyl)-4,5,6,7-tetrahydro-1H-pyrazolo[4,3-c]pyridin-3-yl]phenyl}ethyl)phenol;
3-{4-Chloro-3-[(4-chlorophenyl)ethynyl]phenyl}-1-(3-morpholin-4-ylpropyl)-4,5,6,7-tetrahydro-1H-pyrazolo[4,3-c]pyridine;
8-{3-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-2,8-diazaspiro[4.5]decan-1-one;
4-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}but-3-yn-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}prop-2-yn-1-amine;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl}prop-2-yn-1-yl)benzenesulfonamide;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl]propyl)benzenesulfonamide;
1-[1-(3-{3-[3-(3-Aminoprop-1-yn-1-yl)-4-chlorophenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl 2-{[(3-{2-chloro-5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]phenyl}propyl)amino]sulfonyl}benzoate;
1-[1-(3-{3-[4-Chloro-3-(3-hydroxyprop-1-yn-1-yl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{3-[4-Chloro-3-(4-hydroxybutyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-{4-Chloro-3-[4-(dimethylamino)butyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[1-(3-{3-[4-Chloro-3-(3-hydroxypropyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-{4-Chloro-3-[3-(dimethylamino)propyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methylmethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-N-ethylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenylethynyl)phenyl]methyl}-2-methylpropan-1-amine;
1-[4-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-[(4-chlorophenyl)methyl]methanamine;
3-(3-{[4-(1H-Benzimidazol-2-yl)phenyl]ethynyl]-4-chlorophenyl)-5-(methylsulfonyl)-1-(3-morpholin-4-ylpropyl)-4,5,6,7-tetrahydro-1H-pyrazolo[4,3-c]pyridine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(phenylmethyl)aniline; {[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]amino]acetonitrile;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclopropanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclobutanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclopentanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclohexanamine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(2-phenylethyl)aniline;
1-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1,1-Dimethylethyl (1-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}piperidin-4-yl)carbamate;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-ol;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-amine;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-morpholin-4-ylpropan-2-ol;
1-[4-({2-Chloro-5-[5-(methylsulfonyl)-1-(3-pyrrolidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
Ethyl 1-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxylate;
1-{4-[(2-Chloro-5-{1-[3-(1,4-dioxa-8-azaspiro[4.5]dec-8-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxylic acid;
(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanol;
1′-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-1,4′-bipiperidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methyloxy)phenyl]methyl}methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2,2,2-trifluoroethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(cyclopropylmethyl)methanamine;
(2S)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
1-{4-[(2-Chloro-5-{5-(methylsulfonyl)-1-[3-(4-morpholin-4-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4-methylpiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1-[4-({2-Chloro-5-[5-(methylsulfonyl)-1-{3-[4-(trifluoromethyl)piperidin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)acetamide;
Methyl N-{[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
1-(1-{3-[3-{4-Chloro-3-[(4-{[(2,2,2-trifluoroethyl)amino]methyl]phenyl)ethynyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}prop-2-en-1-amine;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxamide;
Methyl (2S)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-(1-{3-[3-(4-Chloro-3-{[4-({[(1R)-2-hydroxy-1-phenylethyl]aminomethyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1-{4-[(5-{1-[3-(4-Acetylpiperazin-1-yl)propyl]-5-(methylsulfonyl)-4,5,6,7-tetrahydro-1H-pyrazolo[4,3-c]pyridin-3-yl}-2-chlorophenyl)ethynyl]phenyl}-N-[(4-chlorophenyl)methyl]methanamine;
1-{4-[(2-Chloro-5-{1-[3-(4-methylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4,4-dimethylpiperidin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)-2-hydroxyacetamide;
1-{4-[(2-Chloro-5-{1-[3-(4,4-difluoropiperidin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4-fluoropiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanesulfonamide;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-piperidin-1-ylpropan-2-ol;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidin-4-yl)acetamide;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidine-4-carboxamide;
3-(4-Chloro-3-{[2-(trifluoroacetyl)-2,3-dihydro-1H-isoindol-5-yl]ethynyl}phenyl)-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1,1-Dimethylethyl 4-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}piperazine-1-carboxylate;
1-[4-({2-Chloro-5-[5-(methylsulfonyl)-1-(3-piperazin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-4,5,6,7-tetrahydro-1H-pyrazolo[4,3-c]pyridin-1-yl]propyl}piperidin-4-yl)acetamide;
1,1-Dimethylethyl 7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl)ethynyl)-3,4-dihydroisoquinoline-2(1H)-carboxylate;
1,1-Dimethylethyl 7-({2-chloro-5-[1-(2-hydroxy-3-piperidin-1-ylpropyl)-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-[4-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1,1-Dimethylethyl 7-({5-[1-{3-[4-(aminocarbonyl)piperidin-1-yl]-2-hydroxypropyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
1,1-Dimethylethyl 7-({5-[1-{3-[4-(aminocarbonyl)piperidin-1-yl]propyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-3-benzazepine;
1,1-Dimethylethyl {[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
7-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[5-(methylsulfonyl)-1-{3-[4-(phenylcarbonyl)piperazin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
7-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-1,2,3,4-tetrahydroisoquinoline;
(3S)-1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
(3R)-1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
1,1-Dimethylethyl {[2-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[1-{3-[(2R,6S)-2,6-dimethylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholin-2-yl)methanol;
1,1-Dimethylethyl 4-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-1,4-diazepane-1-carboxylate;
1-{4-[(2-Chloro-5-{[1-[3-(1,4-diazepan-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1,1-Dimethylethyl 5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,3-dihydro-2H-isoindole-2-carboxylate;
3-[4-Chloro-3-(2,3-dihydro-1H-isoindol-5-ylethynyl)phenyl]-5-(methylsulfonyl)-1-(3-morpholin-4-ylpropyl)-4,5,6,7-tetrahydro-1H-pyrazolo[4,3-c]pyridine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro-1,6-naphthyridine;
1-{4-[2-(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethyl]phenyl}-N-[(4-chlorophenyl)methyl]methanamine;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-2-one;
1-{4-[(2-Chloro-5-{1-[3-(1,1-dioxidothiomorpholin-4-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(1,4-oxazepan-4-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-(4-{[5-(5-Acetyl-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}phenyl)-N-[(4-chlorophenyl)methyl]methanamine;
3-(4-Chloro-3-{[4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridine-5-carboxamide;
2-[3-{[4-Chloro-3-{[(4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoethanol;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro[1,2,4]triazolo[4,3-a]pyrazine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-3-methylpiperazin-2-one;
2-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
3-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
4-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
3-[3-(1H-Benzimidazol-5-ylethynyl)-4-chlorophenyl]-5-(methylsulfonyl)-1-(3-morpholin-4-ylpropyl)-4,5,6,7-tetrahydro-1H-pyrazolo[4,3-c]pyridine;
1-[4-({5-[5-Acetyl-1-(3-thiomorpholin-4-ylpropyl)-4,5,6,7-tetrahydro-1H-pyrazolo[4,3-c]pyridin-3-yl]-2-chlorophenyl]ethynyl)phenyl]-N-[(4-chlorophenyl)methyl]methanamine;
2-[3-{[4-Chloro-3-{[(4-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridin-5-yl]-2-oxoethanol;
2-[3-{[4-Chloro-3-{[(4-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridin-5-yl]-2-oxoacetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-1,2,3,4-tetrahydroisoquinoline;
1-{4-[(2-Chloro-5-{5-(methylsulfonyl)-1-[3-(4-pyridin-4-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-pyridin-3-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
Methyl 4-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylate;
(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholin-3-yl)methanol;
1-[4-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(3-thienylmethyl)methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-(2-thienyl)ethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[5-methyl-2-(trifluoromethyl)furan-3-yl]methyl}methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-[(4-chlorophenyl)methyl]methanamine; 2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoacetamide;
3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridine-5-carboxamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(3-thienylmethyl)methanamine;
2-[3-{[4-Chloro-3-{[(4-({[(4-chlorophenyl)methyl]amino{methyl)phenyl]ethynyl]phenyl)-1-{3-[(1S,4S)-2-oxa-5-azabicyclo[2.2.1]hept-5-yl]propyl}-1,4,6,7-tetrahydro-5H-pyrazolo[4,3-c]pyridin-5-yl]-2-oxoacetamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-[(4-chlorophenyl)methyl]methanamine;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylic acid;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-(1H-imidazol-4-ylethynyl)phenyl]-5-(methylsulfonyl)-1-(3-morpholin-4-ylpropyl)-4,5,6,7-tetrahydro-1H-pyrazolo[4,3-c]pyridine;
2-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydroisoquinoline;
3-(4-Chloro-3-{[4-(1,3-dihydro-2H-isoindol-2-ylmethyl)phenyl]ethynyl}phenyl)-5-(methylsulfonyl)-1-(3-morpholin-4-ylpropyl)-4,5,6,7-tetrahydro-1H-pyrazolo[4,3-c]pyridine;
(1R)—N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydronaphthalen-1-amine;
(1S,2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl)ethynyl]phenyl]methyl]amino)-2,3-dihydro-1H-inden-1-ol;
1-{3-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-{3-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
(2S)-1-(4,4′-Bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
2-[3-{[4-Chloro-3-{[(4-({[(4-chlorophenyl)methyl]amino(methyl)phenyl]ethynyl}-phenyl)-1-(3-piperidin-1-ylpropyl)-1,4,6,7-tetrahydro-5H-pyrazolo[4,3-c]pyridin-5-yl]-2-oxoacetamide;
1-(3-{5-[Amino(oxo)acetyl]-3-(4-chloro-3-{[4-({[(4-chlorophenyl)methyl]aminomethyl)phenyl]ethynyl]phenyl)-4,5,6,7-tetrahydro-1H-pyrazolo[4,3-c]pyridin-1-yl}propyl)piperidine-4-carboxamide;
2-{3-(4-Chloro-3-{[4-({[(4-chlorophenyl)methyl]amino-4-methyl)phenyl]ethynyl-phenyl)-1-[3-(1,4-dioxa-8-azaspiro[4.5]dec-8-yl)propyl]-1,4,6,7-tetrahydro-5H-pyrazolo[4,3-c]pyridin-5-yl}-2-oxoacetamide;
2-[3-{[4-Chloro-3-{[(4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridin-5-yl]-2-oxoacetamide;
3-(4-Chloro-3-{[4-({[(4-chlorophenyl)methyl]amino(methyl)phenyl]ethynyl}-phenyl)-1-(3-pyrrolidin-1-ylpropyl)-1,4,6,7-tetrahydro-5H-pyrazolo[4,3-c]pyridine-5-carboxamide;
3-(4-Chloro-3-{[4-({[(4-chlorophenyl)methyl]amino(methyl)phenyl]ethynyl]phenyl)-1-{3-[4-(1-oxidopyridin-2-yl)piperazin-1-yl]propyl}-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridine-5-carboxamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(1,3-thiazol-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(1-methyl-1H-imidazol-5-yl)methyl]methanamine;
3-(4-Chloro-3-{[4-({[(4-chlorophenyl)methyl]amino-methyl)phenyl]ethynyl]phenyl)-1-(3-piperidin-1-ylpropyl)-1,4,6,7-tetrahydro-5H-pyrazolo[4,3-c]pyridine-5-carboxamide;
1-{3-[4-(Acetylamino)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-cyclobutylpiperazin-1-yl)propyl]-1,4,6,7-tetrahydro-5H-pyrazolo[4,3-c]pyridine-5-carboxamide;
1-{3-[4-(Aminocarbonyl)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-{[4-({[(4-chlorophenyl)methyl]amino-4-methyl)phenyl]ethynyl]phenyl)-1-[3-(1,4-dioxa-8-azaspiro[4.5]dec-8-yl)propyl]-1,4,6,7-tetrahydro-5H-pyrazolo[4,3-c]pyridine-5-carboxamide;
3-(4-Chloro-3-[4-(3-[(4-chlorophenyl)methyl]amino]methyl)phenyl]ethynyl]phenyl)-1-[3-(4-cyclopropylpiperazin-1-yl)propyl]-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
1,1-Dimethylethyl 7-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-{4-[(2-Chloro-5-{5-(methylsulfonyl)-1-[3-(2-oxa-6-azaspiro[3.3]hept-6-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-2,3-dihydro-1H-inden-1-amine;
(2S)-1-(4,4′-Bipiperidin-1-yl)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]propan-2-ol;
(2S)-1-(4,4′-Bipiperidin-1-yl)-3-{3-[4-chloro-3-(cyclohexylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl}propan-2-ol;
3-(4-Chloro-3-{2-[4-({[(4-chlorophenyl)methyl]amino-methyl)phenyl]ethyl]phenyl)-1-(3-morpholin-4-ylpropyl)-1,4,6,7-tetrahydro-5H-pyrazolo[4,3-c]pyridine-5-carboxamide;
3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
1-[(2S)-3-(4,4′-Bipiperidin-1-yl)-2-hydroxypropyl]-3-{4-chloro-3-[2-(4-chlorophenyl)ethyl]phenyl}-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}methyl)-2,2,2-trifluoroethanamine;
7-{[2-Chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}-2-cyclopropyl-1,2,3,4-tetrahydroisoquinoline;
3-[4-Chloro-3-(1H-pyrrolo[2,3-b]pyridin-6-ylethynyl)phenyl]-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-[(4-methylpiperazin-1-yl)carbonyl]-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
3-(4-Chloro-3-{[4-({[(3-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(2-chlorophenyl)methyl]amino(methyl)phenyl]ethynyl]phenyl)-1-(3-thiomorpholin-4-ylpropyl)-1,4,6,7-tetrahydro-5H-pyrazolo[4,3-c]pyridine-5-carboxamide;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-2-benzazepine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]-N-methylmethanamine;
1,1-Dimethylethyl (1S,4S)-5-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1,1-Dimethylethyl (1R,4R)-5-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1-[4-({2-Chloro-5-[1-{3-[(1S,4S)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(1R,4R)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
2-[3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridin-5-yl]-2-oxoacetamide;
1-[4-({2-Chloro-5-[5-(morpholin-4-ylcarbonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-carboxamide;
N-{2-[3-(4-Chloro-3-{[4-({[(4-chlorophenyl)methyl]amino-3-methyl)phenyl]ethynyl]phenyl)-1-(3-morpholin-4-ylpropyl)-1,4,6,7-tetrahydro-5H-pyrazolo[4,3-c]pyridin-5-yl]-2-oxoethylacetamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl-4-ethynyl)-2-(2,2,2-trifluoroethyl)-1,2,3,4-tetrahydroisoquinoline;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]-N-methylmethanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(cyclopropylmethyl)-1,2,3,4-tetrahydroisoquinoline;
2-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
3-(4-Chloro-3-{[4-([(3-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl]ethynyl)phenyl]methyl]amino)propan-1-ol;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydrofuran-2-ylmethyl)methanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}aniline;
6-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
3-(4-Chloro-3-{[3-({[(2-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(2-fluorophenyl)methyl]amino-3-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-fluorophenyl)methyl]amino-methyl)phenyl]ethynyl(phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(4-fluorophenyl)methyl]amino-3-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-methyl-1,2,3,4-tetrahydroisoquinoline;
6-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-methylpropan-1-amine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(2-methylpropyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methylsulfonyl)phenyl]methyl}methanamine;
1,1-Dimethylethyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1-oxo-3,4-dihydroisoquinoline-2(1H)-carboxylate;
Ethyl [6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinolin-2(1H)-yl]acetate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-prop-2-en-1-yl-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methyl-N-(phenylmethyl)methanamine;
(2R)-1-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-[(3S)-3-methylmorpholin-4-yl]propan-2-ol;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-methyl-N-(phenylmethyl)methanamine;
N-{[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenylethyl)phenyl]methyl}-2-methylpropan-1-amine;
3-(4-Chloro-3-{[4-([(2-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-{[(4-[({[2-(methyloxy)phenyl]methyl]amino)methyl]phenyl]ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-{[(4-[({[3-(methyloxy)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine;
1-[4-({2-Chloro-5-[1-{3-[(3R)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{5-(methylsulfonyl)-1-[3-(1-oxidothiomorpholin-4-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
3-[4-Chloro-3-({4-[({[4-(methyloxy)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
1-[5-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-fluorophenyl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(methyloxy)phenyl]-N-(phenylmethyl)methanamine;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
3-(4-Chloro-3-[4-({[(2-fluorophenyl)methyl]amino]methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(3-fluorophenyl)methyl]amino-methyl)phenyl]ethynyl(phenyl)-1-(3-thiomorpholin-4-ylpropyl)-1,4,6,7-tetrahydro-5H-pyrazolo[4,3-c]pyridine-5-carboxamide;
3-(4-Chloro-3-([4-({[(4-fluorophenyl)methyl]amino]methyl)phenyl]ethynyl]phenyl)-1-(3-thiomorpholin-4-ylpropyl)-1,4,6,7-tetrahydro-5H-pyrazolo[4,3-c]pyridine-5-carboxamide;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-[4-([(4-chlorophenyl)methyl]aminocarbonyl)phenyl]ethynyl]phenyl)-1-(3-thiomorpholin-4-ylpropyl)-1,4,6,7-tetrahydro-5H-pyrazolo[4,3-c]pyridine-5-carboxamide; 3-{4-Chloro-3-[(4-([(morpholin-2-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-({4-[(4R)-4-hydroxy-2-oxopyrrolidin-1-yl]piperidin-1-yl}methyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-[4-Chloro-3-(−4-[({[4-(methylsulfanyl)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-(3-{[4-({[(4-Aminophenyl)methyl]amino]methyl)phenyl]ethynyl}-4-chlorophenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(3R)-3-hydroxypyrrolidin-1-yl]carbonyl}phenyl)ethynyl]phenyl}-1-(3-thiomorpholin-4-ylpropyl)-1,4,6,7-tetrahydro-5H-pyrazolo[4,3-c]pyridine-5-carboxamide;
N-[(2-Chloro-5-{[2-chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}phenyl)methyl]glycine;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(3-hydroxypropyl)benzamide;
3-[4-Chloro-3-({4-chloro-3-[(3-hydroxypropyl)carbamoyl]phenyl(ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(2S)-pyrrolidin-2-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(2S)-pyrrolidin-2-ylmethyl]benzamide;
3-{4-Chloro-3-[(4-chloro-3-[(2S)-pyrrolidin-2-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
tert-Butyl 3-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorobenzyl)oxy]pyrrolidine-1-carboxylate;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(3-hydroxypropyl)benzamide;
2-(3-{4-Chloro-3-[(4-chloro-3-{[(2-hydroxy-2-methylpropyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-[3-{4-Chloro-3-[(4-chloro-3-{[(2-hydroxy-2-methylpropyl)amino]methyl}phenyl)ethynyl]phenyl}-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-({[(1R)-2-hydroxy-1-phenylethyl]amino}methyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(1R)-2-hydroxy-1-phenylethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[3-(methylamino)propyl]benzamide;
2-(3-{4-Chloro-3-({4-chloro-3-[(ethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
2-[3-{[4-Chloro-3-{[(4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
3-(4-Chloro-3-{[4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
2-[3-{[4-Chloro-3-{[(4-chloro-3-(pyrrolidin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)methylcarbamate;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)carbamate;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(3R)-pyrrolidin-3-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(piperidin-3-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(morpholin-2-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
3-{4-Chloro-3-[(4-chloro-3-[(3R)-pyrrolidin-3-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-(4-chloro-3-[(piperidin-3-ylmethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(morpholin-2-ylmethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(3R)-pyrrolidin-3-ylmethyl]benzamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(piperidin-3-ylmethyl)benzamide;
2-[3-{[4-Chloro-3-{[(4-chloro-3-(morpholin-4-ylmethyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-({4-chloro-3-[(cyclopropylamino) methyl]phenylethynyl)phenyl]-1-{3-[(3S)-3-methylmorpholin-4-yl]propyl}-1,4,6,7-tetrahydro-5H-pyrazolo[4,3-c]pyridin-5-yl)-2-oxoacetamide;
3-[4-Chloro-3-({4-chloro-3-[(2-morpholin-4-ylethyl)carbamoyl]phenyl}ethynyl)phenyl]-1-(3-piperidin-1-ylpropyl)-1,4,6,7-tetrahydro-5H-pyrazolo[4,3-c]pyridine-5-carboxamide;
5-[(5-{5-[Amino(oxo)acetyl]-1-(3-piperidin-1-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
2-{3-[4-Chloro-3-{(4-chloro-3-[(ethylamino) methyl]phenylethynyl)phenyl]-1-(3-piperidin-1-ylpropyl)-1,4,6,7-tetrahydro-5H-pyrazolo[4,3-c]pyridin-5-yl}-2-oxoacetamide;
2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]ethanamine;
2-{3-[4-Chloro-3-({4-chloro-3-[(cyclopentylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
3-[4-Chloro-3-({4-chloro-3-[(cyclopropylamino)methyl]phenyl}ethynyl)phenyl]-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopentanamine;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(azetidin-3-ylmethyl)-2-chlorobenzamide;
3-[4-Chloro-3-(4-chloro-3-[(cyclopentylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide; 2-{3-[4-Chloro-3-(−4-chloro-3-[(cyclopropylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
2-[3-{[4-Chloro-3-{[(4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridin-5-yl]-2-oxoacetamide;
3-(4-Chloro-3-{[4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopropanamine;
3-(4-Chloro-3-{[4-chloro-3-(morpholin-4-ylmethyl)phenyl]ethynyl]phenyl)-5-(methylsulfonyl)-1-(3-piperidin-1-ylpropyl)-4,5,6,7-tetrahydro-1H-pyrazolo[4,3-c]pyridine;
3-[4-Chloro-3-{(4-chloro-3-[(cyclopropylamino)methy]phenyl]ethynyl)phenyl]-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(morpholin-4-ylmethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-{(4-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
3-(4-Chloro-3-[(4-{[(2S)-pyrrolidin-2-ylmethyl]carbamoyl]phenyl)ethynyl]phenyl]-1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl]-N-[(2S)-pyrrolidin-2-ylmethyl]benzamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[(2S)-pyrrolidin-2-ylmethyl]benzamide;
3-{4-Chloro-3-[(4-{[3-(methylamino)propyl]carbamoyl}phenyl)ethynyl]phenyl}-1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl]-N-[3-(methylamino)propyl]benzamide; and
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[3-(methylamino)propyl]benzamide;
3-[4-Chloro-3-{(4-chloro-3-[(dimethylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
2-(3-{4-Chloro-3-({4-chloro-3-[(dimethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N,N-dimethylmethanamine;
2-[3-{[4-Chloro-3-{[(4-(hydroxymethyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-[(4-chloro-3-{[(1-methylethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-(3-{4-Chloro-3-[(4-{[(4-chlorobenzyl)amino]methyl-phenyl)ethynyl]phenyl}-1-{3-[(3R)-3-methylmorpholin-4-yl]propyl}-1,4,6,7-tetrahydro-5H-pyrazolo[4,3-c]pyridin-5-yl)-2-oxoacetamide;
2-(3-{4-Chloro-3-({4-{[(4-chlorobenzyl)amino]methyl-phenyl)ethynyl]phenyl}-1-[3-(3-methylmorpholin-4-yl)propyl]-1,4,6,7-tetrahydro-5H-pyrazolo[4,3-c]pyridin-5-yl)-2-oxoacetamide;
and pharmaceutically acceptable salts thereof.

53. A compound selected from the group consisting of:
4-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}butan-1-ol;
5-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}pentan-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}propan-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}propan-1-amine;
1-[1-(3-{3-[3-(3-Aminopropyl)-4-chlorophenyl]-5-(methylsulfonyl)-4,5,6,7-tetrahydro-1H-pyrazolo[4,3-c]pyridin-1-yl}propyl)piperidin-4-yl]pyrrolidin-2-one;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynylamine; and
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propylamine;
and pharmaceutically acceptable salts thereof.

54. A compound as defined in claim 1, wherein said compound is a compound of Formula (I) or a pharmaceutically acceptable salt of a compound of Formula (I).

55. A pharmaceutical composition for treating a disease, disorder, or medical condition mediated by cathepsin S activity, comprising:
(a) an effective amount of at least one chemical entity selected from compounds of Formula (I): 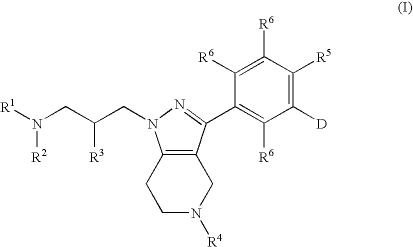
wherein:
R¹ and R² taken together with the nitrogen to which they are attached form a saturated monocyclic heterocycloalkyl group, each group optionally containing one additional heteroatom ring member that is O, S, or NR^{a}, and each group unsubstituted or substituted with one, two, or three R^{b} substituents;
where R^{a} is H, C₁₋₄alkyl, —COC₁₋₄alkyl, —CO(phenyl), or —CO₂C₁₋₄alkyl, or a monocyclic cycloalkyl ring, phenyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R substituent is independently:
i) OH, C₁₋₄alkyl, —C₁₋₄alkyl-OH, CF₃, —NR^{c}R^{d}, halo, —OC₁₋₄alkyl, —COC₁₋₄alkyl, —CO₂C₁₋₄alkyl, —CO₂H, or —CONR^{e}R^{f};
ii) a monocyclic heterocycloalkyl group unsubstituted or substituted with C₁₋₄alkyl, —COC₁₋₄alkyl, —CO₂C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group or monocyclic heteroaryl group, each group unsubstituted or substituted with C₁₋₄alkyl, OH, —OC₁₋₄alkyl, —NR^{c}R^{d}, or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo;
vi) two R^{b} substituents form a methylene or ethylene bridge; or
vii) two R^{b} substituents on adjacent carbons taken together with the carbons to which they are attached form a saturated monocyclic cycloalkyl group or saturated monocyclic heterocycloalkyl group, each group unsubstituted or substituted with C₁₋₄alkyl, OH, —OC₁₋₄alkyl, —NR^{c}R^{d}, or halo;
where R^{c} is H or C₁₋₄alkyl;
R^{d} is H, C₁₋₄alkyl, —COC₁₋₄alkyl, —COC₁₋₄alkyl-OH, —CO₂C₁₋₄alkyl, —CONR^{x}R^{y}, or —SO₂C₁₋₄alkyl;
where R^{x} and R^{y} are each independently H or C₁₋₄alkyl; and
R^{e} and R^{f} are each independently H or C₁₋₄alkyl;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, or —NR^{t}R^{u}; —COCF₃; —CO-(monocyclic heteroaryl); —CO—(C-linked monocyclic heterocycloalkyl); —CO-(phenyl); —SO₂C₁₋₄alkyl; —SO₂CF₃; —SO₂NR^{t}R^{u}; —CONR^{t}R^{u}; —COCO₂C₁₋₄alkyl; or —COCONR^{t}R^{u};
where R^{t} and R^{u} are each independently H, C₁₋₄alkyl, or —COC₁₋₄alkyl; or R^{t} and R^{u} taken together with the nitrogen to which they are attached form a monocyclic heterocycloalkyl ring;
R⁵ is halo or CF₃;
each R⁶ is independently H or F;
D is —C≡C—R⁷, —CH═CH—R³, —(CH₂)₂₋₃—R³, or —(CH₂)₃₋₅—R⁹;
where R⁷ is:
I) a C₁₋₄alkyl group unsubstituted or substituted with OH, —OC₁₋₄alkyl, —NR^{g}R^{h}; phenyl, or phenoxy, each phenyl or phenoxy being unsubstituted or substituted with C₁₋₄alkyl, OH, —OC₁₋₄alkyl, halo, or CF₃;
where R^{g} and R^{h} are each independently H, C₁₋₄alkyl, —COC₁₋₄alkyl, —COphenyl, —CO₂C₁₋₄alkyl, —SO₂C₁₋₄alkyl, or —SO₂-phenyl; or R^{g} and R^{h} taken together with the nitrogen to which they are attached form a monocyclic heterocycloalkyl group; or
II) a monocyclic cycloalkyl group, phenyl group, or monocyclic heteroaryl group, each group unsubstituted or substituted with one or two R^{k} substituents;
where R⁸ is a phenyl group or monocyclic heteroaryl group, each group unsubstituted or substituted with one or two R^{k} substituents;
where R⁹ is OH or —NRⁿR^{o};
where Rⁿ is H or C₁₋₄alkyl; and
R^{o} is H, C₁₋₄alkyl, monocyclic cycloalkyl, —COC₁₋₄alkyl, —COphenyl, —CO₂C₁₋₄alkyl, —SO₂C₁₋₄alkyl, —SO₂-phenyl, —SO₂-benzyl, or —SO₂NR^{p}R^{g}, each phenyl or benzyl group being unsubstituted or substituted with one or two R^{k} substituents;
where R^{p} and R^{g} are each independently H or C₁₋₄alkyl;
or Rⁿ and R^{o} taken together with the nitrogen to which they are attached form a monocyclic saturated heterocycloalkyl ring unsubstituted or substituted with C₁₋₄alkyl, OH, —OC₁₋₄alkyl, halo, or CF₃;
where each R^{k} substituent in D is independently:
a) a C₁₋₄alkyl group unsubstituted or substituted with OH, —OC₁₋₄alkyl, —OC₁₋₄alkyl-OH, halo, —CO₂C₁₋₄alkyl, CO₂H, CN, —NR^{r}R^{s}, —N(R^{r})COphenyl, —N(R^{r})SO₂C₁₋₄alkyl, —N(R^{r}) SO₂-phenyl, —SO₂C₁₋₄alkyl, phenyl, or phenoxy; 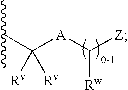
b) a substituent of formula where each R^{v} is independently H or C₁₋₄alkyl, or both R^{v} substituents together form a carbonyl;
R^{w} is H, C₁₋₄alkyl, —CH₂OH, or —CO₂C₁₋₄alkyl;
A is O or NR^{aa};
where R^{aa} is H or C₁₋₄alkyl; and
Z is a phenyl group, benzyl group, cycloalkyl group, heterocycloalkyl group, heteroaryl group, or —CH₂— (heteroaryl) group, each group unsubstituted or substituted with one or two substituents independently selected from the group consisting of C₁₋₄alkyl, CF₃, halo, OH, —OC₁₋₄alkyl, —OCF₃, —OCHF₂, NR^{dd}R^{ee}, —CO₂C₁₋₄alkyl, —SC₁₋₄alkyl, and —SO₂C₁₋₄alkyl;
where R^{dd} and R^{ee} are each independently H or C₁₋₄alkyl;
c) two adjacent R^{k} substituents taken together with the carbons to which they are attached form a fused phenyl ring, monocyclic heteroaryl ring, monocyclic heterocycloalkyl ring, or monocyclic cycloalkyl ring, each fused ring unsubstituted or substituted with: C₁₋₄alkyl, —C₁₋₄alkyl-CF₃, —C₁₋₄alkyl-OH, —C₁₋₄alkyl-CO₂C₁₋₄alkyl, CF₃, C₂₋₄alkenyl, halo, OH, —OC₁₋₄alkyl, —COC₁₋₄alkyl, —COCF₃, —CO₂C₁₋₄alkyl, —CO₂H, —CONR^{ff}R^{gg}, or —SO₂C₁₋₄alkyl; or with a cycloalkyl group, —CH₂-(cycloalkyl) group, or benzyl group, each group unsubstituted or substituted with C₁₋₄alkyl, OH, —OC₁₋₄alkyl, halo, or CF₃;
where R^{ff} and R^{gg} are each independently H or C₁₋₄alkyl, or R^{ff} and R^{gg} taken together with the nitrogen to which they are attached form a monocyclic heterocycloalkyl ring, unsubstituted or substituted with C₁₋₄alkyl or OH; or
d) OH; —OC₁₋₄alkyl; halo; CF₃; —CHO; —CO₂C₁₋₄alkyl; CO₂H; CN; —NO₂; —CONR^{r}R^{s}, —NR^{r}R^{s}; —N(R^{r})_phenyl; —N(R^{r})-benzyl; —N(R^{r})-phenethyl; —N(R^{r})COC₁₋₄-alkyl; —N (R^{r}) CO-phenyl; —N(R^{r})SO₂C₁₋₄alkyl; —N (R^{r})SO₂-phenyl; —SO₂C₁₋₄alkyl; phenoxy; or a heteroaryl group; where each phenyl, benzyl, phenethyl, phenoxy, or heteroaryl group is unsubstituted or substituted with C₁₋₄alkyl, OH, —OC₁₋₄alkyl, halo, or CF₃;
where R^{r} is H, C₁₋₄alkyl, C₂₋₄alkyl-OH; and
R^{s} is H, C₁₋₄alkyl, —C₁₋₄alkyl-CF₃, —C₁₋₄alkyl-CN, —C₂₋₄alkyl-OH, —C₂₋₄alkyl-NR^{bb}R^{cc}, —C₁₋₄alkylCO₂C₁₋₄alkyl, —C₁₋₄alkylCO₂H, C₃₋₄alkenyl, —COC₁₋₄alkyl, or —CO₂C₁₋₄alkyl;
where R^{b} is H or C₁₋₄alkyl; and
R^{cc} is H, C₁₋₄alkyl, —COC₁₋₄alkyl, or —CO₂C₁₋₄alkyl;
or R^{bb} and R^{cc} taken together with the nitrogen to which they are attached from a monocyclic heterocycloalkyl ring;
or R^{r} and R^{s} taken together with the nitrogen to which they are attached form a heterocycloalkyl group unsubstituted or substituted with C₁₋₄alkyl, OH, —OC₁₋₄alkyl, halo, CF₃, or a monocyclic heterocycloalkyl ring unsubstituted or substituted with OH;
and pharmaceutically acceptable salts, prodrugs, and metabolites thereof; and
(b) a pharmaceutically acceptable excipient.

56. A pharmaceutical composition according to claim 55, wherein said chemical entity is selected from the group consisting of:
3-[4-Chloro-3-(1H-indol-5-ylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[2-(1H-indol-5-yl)-ethyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
4-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-butyric acid;
3-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-propionic acid;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
3-(4-Chloro-3-thiophen-2-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3,4-dichloro-phenylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[4-(4-iodo-phenoxy)-phenylethynyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
3-[4-Chloro-3-(4-phenoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[3-(4-Bromo-phenylethynyl)-4-chloro-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzoic acid;
3-(4-Chloro-3-pyridin-4-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-3-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-2-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-thiophen-3-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
3-[4-Chloro-3-(4-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-p-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(4-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2,4-difluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-o-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzaldehyde;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenylamine;
3-(4-Chloro-3-phenylethynyl-phenyl)-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-carbamic acid tert-butyl ester;
3-[4-Chloro-3-(3-phenyl-prop-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-phenyl-but-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(5-phenyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-yn-1-ol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-but-3-yn-1-ol;
5-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-pent-4-yn-1-ol;
3-(4-Chloro-3-hex-1-ynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-cyclohexylethynyl-phenyl)-5-methanesulfonyl-1-(3-morpholin-4-yl-propyl)-4,5,6,7-tetrahydro-1H-pyrazolo[4,3-c]pyridine;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-diethyl-amine;
3-{4-Chloro-3-[3-(1,1-dioxo-1λ⁶-thiomorpholin-4-yl)-prop-1-ynyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(4-methyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-phenoxy-prop-1-ynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzenesulfonamide;
3-{4-Chloro-3-[2-(4-phenoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenol;
3-{4-Chloro-3-[2-(4-chloro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-{4-Chloro-3-[2-(4-methoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-{4-Chloro-3-[2-(2,4-difluoro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(2-o-tolyl-ethyl)-phenyl]-5-methanesulfonyl-1-(3-morpholin-4-yl-propyl)-4,5,6,7-tetrahydro-1H-pyrazolo[4,3-c]pyridine;
[4-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenyl]-methanol;
3-(4-Chloro-3-phenethyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-phenyl-propyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetamide;
3-(4-Chloro-3-Z-styryl-phenyl)-5-methanesulfonyl-1-(3-morpholin-4-yl-propyl)-4,5,6,7-tetrahydro-1H-pyrazolo[4,3-c]pyridine;
3-(4-Chloro-3-E-styryl-phenyl)-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzylamine;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methanesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-acetamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzamide;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-(4-methyl-benzyl)-amine;
(4-Chloro-benzyl)-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methyl-amine;
3-[4-Chloro-3-(4-pyrrolidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-piperidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-methanesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-C-phenyl-methanesulfonamide;
3,4-Dichloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
4-Chloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methyl-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl]-propyl)-4-methoxy-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-N,N-dimethyl-sulfamoylurea;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-acetamide;
2-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propylsulfamoyl}-benzoic acid;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-nitro-benzenesulfonamide;
3-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-4-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methoxy-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methyl-benzenesulfonamide;
2-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-nitro-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-cyano-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methanesulfonyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methanesulfonyl-benzenesulfonamide;
1-[1-(3-{3-[4-Chloro-3-(3-pyrrolidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-piperidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(3-methyl-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[3-(4,4-difluoro-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(3-morpholin-4-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(4-methyl-piperazin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(3-Azepan-1-yl-propyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-cyclopentylamino-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-pyrrolidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-piperidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[4-(3-methyl-piperidin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[4-(4-methyl-piperazin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(4-morpholin-4-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Azepan-1-yl-butyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-cyclopentylamino-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
8-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-2,8-diaza-spiro[4.5]decan-1-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid amide;
3-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-1,3-dihydro-imidazo[4,5-b]pyridin-2-one;
[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
1-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-3-morpholin-4-yl-propan-2-ol;
2-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-cyclopentanone;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-morpholin-4-yl-propan-2-ol;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperidin-1-yl-propan-2-ol;
3-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-,3-dihydro-imidazo[4,5-b]pyridin-2-one;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid amide;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-pyrrolidin-1-yl-propan-2-ol;
[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
4-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperazine-1-carboxylic acid tert-butyl ester;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperazin-1-yl-propan-2-ol;
1-(4-Amino-piperidin-1-yl)-3-{3-[4-chloro-3-(4-chloro-phenylethynyl)-phenyl]-5-methanesulfonyl-4,5,6,7-tetrahydro-pyrazolo[4,3-c]pyridin-1-yl}-propan-2-ol;
1-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-3-piperidin-1-yl-propan-2-ol;
1-(3-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-propyl)-piperidine-4-carboxylic acid amide;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-oxo-acetic acid methyl ester;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-pyridin-2-yl-methanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-furan-2-yl-methanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2,2,2-trifluoro-ethanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-fluoro-ethanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-(tetrahydro-furan-2-yl)-methanone;
Acetic acid 2-[3-[4-chloro-3-(4-chloro-phenylethynyl)-phenyl]-1-(3-morpholin-4-yl-propyl)-1,4,6,7-tetrahydro-pyrazolo[4,3-c]pyridin-5-yl]-2-oxo-ethyl ester;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-hydroxy-ethanone;
3-[4-Chloro-3-(4-chloro-phenylethynyl)-phenyl]-1-(3-morpholin-4-yl-propyl)-1,4,6,7-tetrahydro-pyrazolo[4,3-c]pyridine-5-carboxylic acid amide;
3-{4-Chloro-3-[2-(4-chloro-phenyl)-ethyl]-phenyl}-1-(3-morpholin-4-yl-propyl)-1,4,6,7-tetrahydro-pyrazolo[4,3-c]pyridine-5-carboxylic acid amide;
1-{1-[3-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-pyrrolidin-2-one;
1-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-3-morpholin-4-yl-propan-2-ol;
8-[3-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-2-hydroxy-propyl]-2,8-diaza-spiro[4.5]decan-1-one; and
1-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-3-morpholin-4-yl-propan-2-ol;
and pharmaceutically acceptable salts thereof.

57. A pharmaceutical composition according to claim 55, wherein said chemical entity is selected from the group consisting of:
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoacetamide;
3-(4-Chloro-3-{[4-({[(4-chlorophenyl)methyl]amino(methyl)phenyl]ethynyl]phenyl)-1-(3-morpholin-4-ylpropyl)-1,4,6,7-tetrahydro-5H-pyrazolo[4,3-c]pyridine-5-carboxamide;
2-[3-(4-Chloro-3-{[4-({[(4-chlorophenyl)methyl]amino]methyl)phenyl]ethynyl]phenyl)-1-(3-morpholin-4-ylpropyl)-1,4,6,7-tetrahydro-5H-pyrazolo[4,3-c]pyridin-5-yl]-N,N-dimethyl-2-oxoacetamide;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-sulfonamide;
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoethanamine;
3-(4-Chloro-3-{[4-({[(4-chlorophenyl)methyl]amino]methyl)phenyl]ethynyl]phenyl)-N-methyl-1-(3-morpholin-4-ylpropyl)-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-pyridin-2-ylpiperazin-1-yl)propyl]-1,4,6,7-tetrahydro-5H-pyrazolo[4,3-c]pyridine-5-carboxamide; 2-(3-{4-Chloro-3-[(4-[(4-chlorobenzyl)amino]methyl-phenyl)ethynyl]phenyl}-1-{3-[(3S,5S)-3,5-dimethylmorpholin-4-yl]propyl}-1,4,6,7-tetrahydro-5H-pyrazolo[4,3-c]pyridin-5-yl)-2-oxoacetamide;
2-[3-{[4-Chloro-3-{[(4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-{3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridin-5-yl}-2-oxoacetamide;
3-(4-Chloro-3-{[4-({[(4-chlorophenyl)methyl]amino(methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(pyridin-3-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-{[(pyridin-3-ylmethyl)amino]carbonyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-{[(piperidin-4-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[4-(2-oxopyrrolidin-1-yl)piperidin-1-yl]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
1-[4-({2-Chloro-5-[1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-({4-chloro-3-[(ethylamino)methyl]phenyl}ethynyl)phenyl]-1-(3-piperidin-1-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(2-morpholin-4-ylethyl)benzamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(2-morpholin-4-ylethyl)benzamide;
3-{4-Chloro-3-[(4-{[(4-chlorobenzyl)amino]methyl]phenyl)ethynyl]phenyl-1-{3-[(3aR,6aS)-tetrahydro-1H-furo[3,4-c]pyrrol-5(3H)-yl]propyl}-1,4,6,7-tetrahydro-5H-pyrazolo[4,3-c]pyridine-5-carboxamide;
3-(4-Chloro-3-{[3-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoethanol;
3-(4-Chloro-3-{[4-([(4-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(1-methylethyl)oxy]phenyl}methyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-({4-[({[4-(dimethylamino)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(difluoromethyl)oxy]phenyl]methyl)amino]methylphenyl)ethynyl]phenyl}-1-(3-thiomorpholin-4-ylpropyl)-1,4,6,7-tetrahydro-5H-pyrazolo[4,3-c]pyridine-5-carboxamide;
1-{4-[(2-Chloro-5-{5-(methylsulfonyl)-1-[3-(4-pyridin-2-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperidin-1-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-[4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)benzaldehyde;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydro-2H-pyran-4-ylmethyl)methanamine;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(tetrahydro-2H-pyran-4-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
Methyl N-{[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycine;
2-(1,1-Dimethylethyl) 3-methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-methyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N,N-dimethyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
Methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
Methyl (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
2-(1,1-Dimethylethyl) 3-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
2-(1,1-Dimethylethyl) 1-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-1,2(1H)-dicarboxylate;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-1-carboxylate;
(2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
Methyl (2R)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3,4-dichlorophenyl)methyl]methanamine;
(1S,2R)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl]phenyl)ethynyl]phenyl}methyl)amino]-2,3-dihydro-1H-inden-1-ol;
(1R,2S)-1-[({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl methyl)amino]-2,3-dihydro-1H-inden-2-ol;
(1R)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1S)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1R)—N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(2S)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)amino]-2-phenylethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
Methyl N-{[5-({5-[5-(aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl}ethynyl)-2-chlorophenyl]methyl}glycinate;
N-{[5-({5-[5-(Aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl]ethynyl)-2-chlorophenyl]methyl]glycine;
3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridine-5-carboxylic acid amide;
3-{4-Chloro-3-[(4-chloro-3-{[(tetrahydrofuran-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
3-{4-Chloro-3-[(4-chloro-3-{[(phenylmethyl)amino]methyl(phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-methyl-1,2,3,4-tetrahydroisoquinoline;
N-[1-(3-{3-[4-Chloro-3-(1,2,3,4-tetrahydroisoquinolin-7-ylethynyl)phenyl]-5-(methylsulfonyl)-4,5,6,7-tetrahydro-1H-pyrazolo[4,3-c]pyridin-1-yl}propyl)piperidin-4-yl]acetamide;
7-(2-{2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethyl)-1,2,3,4-tetrahydroisoquinoline;
7-[2-(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethyl]-1,2,3,4-tetrahydroisoquinoline;
1,1-Dimethylethyl 1′-[(2S)-3-[3-[4-chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
1,1-Dimethylethyl 1′-[(2S)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl]-2-hydroxypropyl]-4,4′-bipiperidine-1-carboxylate;
1,1-Dimethylethyl 1′-[(2S)-3-{3-[4-chloro-3-(cyclohexylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-(2S)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]-2-hydroxypropyl-4,4′-bipiperidine-1-carboxylate;
1,1-Dimethylethyl 1′-[(2S)-3-[5-(aminocarbonyl)-3-[4-chloro-3-[(4-chlorophenyl)ethynyl]phenyl}-4,5,6,7-tetrahydro-1H-pyrazolo[4,3-c]pyridin-1-yl]-2-hydroxypropyl]-4,4′-bipiperidine-1-carboxylate;
1-[(2S)-3-(4,4′-Bipiperidin-1-yl)-2-hydroxypropyl]-3-{4-chloro-3-[(4-chlorophenyl)ethynyl]phenyl}-1,4,6,7-tetrahydro-5H-pyrazolo[4,3-c]pyridine-5-carboxamide;
(2S)-1-(4,4′-Bipiperidin-1-yl)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl}propan-2-ol;
(2S)-1-[3-[4-Chloro-3-[(4-chlorophenyl)ethynyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(2S)-1-(1′-Acetyl-4,4′-bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
1,1-Dimethylethyl 1′-[(2S)-3-[5-(aminocarbonyl)-3-[4-chloro-3-[2-(4-chlorophenyl)ethyl]phenyl}-4,5,6,7-tetrahydro-1H-pyrazolo[4,3-c]pyridin-1-yl]-2-hydroxypropyl]-4,4′-bipiperidine-1-carboxylate;
2-[3-(4-Chloro-3-{2-[4-({[(4-chlorophenyl)methyl]amino{methyl)phenyl]ethyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
3-(4-Chloro-3-{2-[4-({[(4-chlorophenyl)methyl]amino{methyl)phenyl]ethyl]phenyl)-1-{3-[(3S)-3-methylmorpholin-4-yl]propyl}-1,4,6,7-tetrahydro-5H-pyrazolo[4,3-c]pyridine-5-carboxamide;
1,1-Dimethylethyl 1′-{(2S)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
(2S)-1-(4,4′-Bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-(1′-Acetyl-4,4′-bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-[3-{4-Chloro-3-[2-(4-chlorophenyl)ethyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(S)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(S)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(S)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(2-{3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
2-{3-(4-Chloro-3-{4-[(4-chloro-benzylamino)-methyl]-phenylethynyl}-phenyl)-1-[(2R)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
(S)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
2-{3-[4-Chloro-3-(4-chloro-3-{[((2S)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
2-{3-[4-Chloro-3-(4-chloro-3-{[((2R)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-cyclopentylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-propylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[3-(3-{[Bis-(2-hydroxy-ethyl)-amino]-methyl}-4-chloro-phenylethynyl)-4-chloro-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-5-(5-{5-Aminooxalyl-1-[3-(3-methyl-morpholin-4-yl)-propyl]-4,5,6,7-tetrahydro-1H-pyrazolo[4,3-c]pyridin-3-yl}-2-chloro-phenylethynyl)-2-chloro-N-(3-methylamino-propyl)-benzamide;
(S)-2-[3-[4-Chloro-3-[4-chloro-3-(3-hydroxy-propoxymethyl)-phenylethynyl]-phenyl}-1-[3-(3-methyl-morpholin-4-yl)-propyl]-1,4,6,7-tetrahydro-pyrazolo[4,3-c]pyridin-5-yl}-2-oxo-acetamide;
(S)-2-[3-[4-Chloro-3-[4-chloro-3-(pyrrolidin-3-yloxymethyl)-phenylethynyl]-phenyl]-1-[3-(3-methyl-morpholin-4-yl)-propyl]-1,4,6,7-tetrahydro-pyrazolo[4,3-c]pyridin-5-yl]-2-oxo-acetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-(1-methylethyl)-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
2-(3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
2-(3-{4-Chloro-3-[(4-chloro-3-{[(phenylmethyl)amino]methyl{phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-(2-{2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine;
2-(3-{4-Chloro-3-[(4-chloro-3-{[(pyridin-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-(phenylmethyl)methanamine;
3-{4-Chloro-3-[(4-chloro-3-[(phenylmethyl)amino]methyl}phenyl)ethynyl]phenyl}-[(3-thiomorpholin-4-ylpropyl)-1,4,6,7-tetrahydro-5H-pyrazolo[4,3-c]pyridine-5-carboxamide;
3-{4-Chloro-3-[(4-chloro-3-{[(pyridin-2-ylmethyl)amino]methyl-phenyl)ethynyl]phenyl}-1-(3-thiomorpholin-4-ylpropyl)-1,4,6,7-tetrahydro-5H-pyrazolo[4,3-c]pyridine-5-carboxamide;
2-({[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]methyl]amino)ethanol;
Methyl N-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)methyl]glycinate; and
2-(3-{4-Chloro-3-[(4-chloro-3-{[(2-hydroxyethyl)amino]methylphenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
and pharmaceutically acceptable salts thereof.

58. A pharmaceutical composition according to claim 55, wherein said chemical entity is selected from the group consisting of:
3-(2-{3-[5-(Methylsulfonyl)-1-(3-morpholin-4-ylpropyl)-4,5,6,7-tetrahydro-1H-pyrazolo[4,3-c]pyridin-3-yl]phenyl}ethyl)phenol;
3-{4-Chloro-3-[(4-chlorophenyl)ethynyl]phenyl}-1-(3-morpholin-4-ylpropyl)-4,5,6,7-tetrahydro-1H-pyrazolo[4,3-c]pyridine;
8-{3-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-2,8-diazaspiro[4.5]decan-1-one;
4-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}but-3-yn-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}prop-2-yn-1-amine;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl}prop-2-yn-1-yl)benzenesulfonamide;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl]propyl)benzenesulfonamide;
1-[1-(3-{3-[3-(3-Aminoprop-1-yn-1-yl)-4-chlorophenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl 2-{[(3-{2-chloro-5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]phenyl}propyl)amino]sulfonyl}benzoate;
1-[1-(3-{3-[4-Chloro-3-(3-hydroxyprop-1-yn-1-yl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{3-[4-Chloro-3-(4-hydroxybutyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-[4-Chloro-3-[4-(dimethylamino)butyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[1-(3-{3-[4-Chloro-3-(3-hydroxypropyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-[4-Chloro-3-[3-(dimethylamino)propyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methylmethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-N-ethylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenylaethynyl)phenyl]methyl}-2-methylpropan-1-amine;
1-[4-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-[(4-chlorophenyl)methyl]methanamine;
3-(3-{[4-(1H-Benzimidazol-2-yl)phenyl]ethynyl]-4-chlorophenyl)-5-(methylsulfonyl)-1-(3-morpholin-4-ylpropyl)-4,5,6,7-tetrahydro-1H-pyrazolo[4,3-c]pyridine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(phenylmethyl)aniline; {[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]amino]acetonitrile;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclopropanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclobutanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclopentanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclohexanamine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(2-phenylethyl)aniline;
1-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1,1-Dimethylethyl (1-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)carbamate;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-ol;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-amine;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-morpholin-4-ylpropan-2-ol;
1-[4-({2-Chloro-5-[5-(methylsulfonyl)-1-(3-pyrrolidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
Ethyl 1-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxylate;
1-{4-[(2-Chloro-5-{1-[3-(1,4-dioxa-8-azaspiro[4.5]dec-8-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxylic acid;
(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanol;
1′-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-1,4′-bipiperidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methyloxy)phenyl]methyl}methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2,2,2-trifluoroethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(cyclopropylmethyl)methanamine;
(2S)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
1-{4-[(2-Chloro-5-{5-(methylsulfonyl)-1-[3-(4-morpholin-4-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4-methylpiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1-[4-({2-Chloro-5-[5-(methylsulfonyl)-1-{3-[4-(trifluoromethyl)piperidin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)acetamide;
Methyl N-{[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
1-(1-{3-[3-{4-Chloro-3-[(4-{[(2,2,2-trifluoroethyl)amino]methyl]phenyl)ethynyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}prop-2-en-1-amine;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxamide;
Methyl (2S)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-(1-{3-[3-(4-Chloro-3-{[4-({[(1R)-2-hydroxy-1-phenylethyl]aminomethyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1-{4-[(5-{1-[3-(4-Acetylpiperazin-1-yl)propyl]-5-(methylsulfonyl)-4,5,6,7-tetrahydro-1H-pyrazolo[4,3-c]pyridin-3-yl}-2-chlorophenyl)ethynyl]phenyl}-N-[(4-chlorophenyl)methyl]methanamine;
1-{4-[(2-Chloro-5-{1-[3-(4-methylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4,4-dimethylpiperidin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)-2-hydroxyacetamide;
1-{4-[(2-Chloro-5-{1-[3-(4,4-difluoropiperidin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4-fluoropiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanesulfonamide;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-piperidin-1-ylpropan-2-ol;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidin-4-yl)acetamide;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidine-4-carboxamide;
3-(4-Chloro-3-{[2-(trifluoroacetyl)-2,3-dihydro-1H-isoindol-5-yl]ethynyl}phenyl)-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1,1-Dimethylethyl 4-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}piperazine-1-carboxylate;
1-[4-({2-Chloro-5-[5-(methylsulfonyl)-1-(3-piperazin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-4,5,6,7-tetrahydro-1H-pyrazolo[4,3-c]pyridin-1-yl]propyl}piperidin-4-yl)acetamide;
1,1-Dimethylethyl 7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl)ethynyl)-3,4-dihydroisoquinoline-2(1H)-carboxylate;
1,1-Dimethylethyl 7-({2-chloro-5-[1-(2-hydroxy-3-piperidin-1-ylpropyl)-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-[4-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1,1-Dimethylethyl 7-({5-[1-{3-[4-(aminocarbonyl)piperidin-1-yl]-2-hydroxypropyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
1,1-Dimethylethyl 7-({5-[1-{3-[4-(aminocarbonyl)piperidin-1-yl]propyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-3-benzazepine;
1,1-Dimethylethyl {[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
7-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[5-(methylsulfonyl)-1-{3-[4-(phenylcarbonyl)piperazin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
7-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-1,2,3,4-tetrahydroisoquinoline;
(3S)-1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
(3R)-1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
1,1-Dimethylethyl {[2-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[1-{3-[(2R,6S)-2,6-dimethylmorpholin-4-yl]propyl]-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholin-2-yl)methanol;
1,1-Dimethylethyl 4-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-1,4-diazepane-1-carboxylate;
1-{4-[(2-Chloro-5-{1-[3-(1,4-diazepan-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1,1-Dimethylethyl 5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,3-dihydro-2H-isoindole-2-carboxylate;
3-[4-Chloro-3-(2,3-dihydro-1H-isoindol-5-ylethynyl)phenyl]-5-(methylsulfonyl)-1-(3-morpholin-4-ylpropyl)-4,5,6,7-tetrahydro-1H-pyrazolo[4,3-c]pyridine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro-1,6-naphthyridine;
1-{4-[2-(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethyl]phenyl}-N-[(4-chlorophenyl)methyl]methanamine;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-2-one;
1-{4-[(2-Chloro-5-{1-[3-(1,1-dioxidothiomorpholin-4-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(1,4-oxazepan-4-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-(4-{[5-(5-Acetyl-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}phenyl)-N-[(4-chlorophenyl)methyl]methanamine;
3-(4-Chloro-3-{[4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridine-5-carboxamide;
2-[3-{[4-Chloro-3-{[(4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoethanol;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro[1,2,4]triazolo[4,3-a]pyrazine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
4-{3-[3-(4-Chloro-3-{[4-({[(4-chlorophenyl)methyl]amino]methyl)phenylethynyl]phenyl)-5-(methylsulfonyl)-4,5,6,7-tetrahydro-1H-pyrazolo[4,3-c]pyridin-1-yl]propyl}-3-methylpiperazin-2-one;
2-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
3-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
4-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
3-[3-(1H-Benzimidazol-5-ylethynyl)-4-chlorophenyl]-5-(methylsulfonyl)-1-(3-morpholin-4-ylpropyl)-4,5,6,7-tetrahydro-1H-pyrazolo[4,3-c]pyridine;
1-[4-({5-[5-Acetyl-1-(3-thiomorpholin-4-ylpropyl)-4,5,6,7-tetrahydro-1H-pyrazolo[4,3-c]pyridin-3-yl]-2-chlorophenyl]ethynyl)phenyl]-N-[(4-chlorophenyl)methyl]methanamine;
2-[3-(4-Chloro-3-{[4-({[(4-chlorophenyl)methyl]amino]methyl)phenyl]ethynyl]phenyl)-1-(3-thiomorpholin-4-ylpropyl)-1,4,6,7-tetrahydro-5H-pyrazolo[4,3-c]pyridin-5-yl]-2-oxoethanol;
2-[3-{[4-Chloro-3-{[(4-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridin-5-yl]-2-oxoacetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-1,2,3,4-tetrahydroisoquinoline;
1-{4-[(2-Chloro-5-{5-(methylsulfonyl)-1-[3-(4-pyridin-4-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-pyridin-3-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
Methyl 4-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylate;
(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholin-3-yl)methanol;
1-[4-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(3-thienylmethyl)methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-(2-thienyl)ethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[5-methyl-2-(trifluoromethyl)furan-3-yl]methyl}methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-[(4-chlorophenyl)methyl]methanamine;
2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoacetamide;
3-(4-Chloro-3-[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridine-5-carboxamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(3-thienylmethyl)methanamine;
2-[3-{[4-Chloro-3-{[(4-({[(4-chlorophenyl)methyl]amino{methyl)phenyl]ethynyl]phenyl)-1-{3-[(1S,4S)-2-oxa-5-azabicyclo[2.2.1]hept-5-yl]propyl}-1,4,6,7-tetrahydro-5H-pyrazolo[4,3-c]pyridin-5-yl]-2-oxoacetamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-[(4-chlorophenyl)methyl]methanamine;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylic acid;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-(1H-imidazol-4-ylethynyl)phenyl]-5-(methylsulfonyl)-1-(3-morpholin-4-ylpropyl)-4,5,6,7-tetrahydro-1H-pyrazolo[4,3-c]pyridine;
2-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydroisoquinoline;
3-(4-Chloro-3-{[4-(1,3-dihydro-2H-isoindol-2-ylmethyl)phenyl]ethynyl}phenyl)-5-(methylsulfonyl)-1-(3-morpholin-4-ylpropyl)-4,5,6,7-tetrahydro-1H-pyrazolo[4,3-c]pyridine;
(1R)—N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydronaphthalen-1-amine;
(1S,2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2,3-dihydro-1H-inden-1-ol;
1-{3-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{3-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
(2S)-1-(4,4′-Bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
2-[3-{[4-Chloro-3-{[(4-({[(4-chlorophenyl)methyl]amino(methyl)phenyl]ethynyl}-phenyl)-1-(3-piperidin-1-ylpropyl)-1,4,6,7-tetrahydro-5H-pyrazolo[4,3-c]pyridin-5-yl]-2-oxoacetamide;
1-(3-{5-[Amino(oxo)acetyl]-3-(4-chloro-3-{[4-({[(4-chlorophenyl)methyl]aminomethyl)phenyl]ethynyl]phenyl)-4,5,6,7-tetrahydro-1H-pyrazolo[4,3-c]pyridin-1-yl}propyl)piperidine-4-carboxamide;
2-{3-(4-Chloro-3-{[4-({[(4-chlorophenyl)methyl]amino-4-methyl)phenyl]ethynyl-phenyl)-1-[3-(1,4-dioxa-8-azaspiro[4.5]dec-8-yl)propyl]-1,4,6,7-tetrahydro-5H-pyrazolo[4,3-c]pyridin-5-yl}-2-oxoacetamide;
2-[3-(4-Chloro-3-{[4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridin-5-yl]-2-oxoacetamide;
3-(4-Chloro-3-{[4-({[(4-chlorophenyl)methyl]amino-methyl)phenyl]ethynyl]phenyl)-1-(3-pyrrolidin-1-ylpropyl)-1,4,6,7-tetrahydro-5H-pyrazolo[4,3-c]pyridine-5-carboxamide;
3-(4-Chloro-3-{[4-({[(4-chlorophenyl)methyl]amino(methyl)phenyl]ethynyl]phenyl)-1-{3-[4-(1-oxidopyridin-2-yl)piperazin-1-yl]propyl}-1,4,6,7-tetrahydro-5H-pyrazolo[4,3-c]pyridine-5-carboxamide;
3-(4-Chloro-3-[4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridine-5-carboxamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(1,3-thiazol-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(1-methyl-1H-imidazol-5-yl)methyl]methanamine;
3-(4-Chloro-3-{[4-({[(4-chlorophenyl)methyl]amino(methyl)phenyl]ethynyl]phenyl)-1-(3-piperidin-1-ylpropyl)-1,4,6,7-tetrahydro-5H-pyrazolo[4,3-c]pyridine-5-carboxamide;
1-{3-[4-(Acetylamino)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-[4-(3-[(4-chlorophenyl)methyl]amino]methyl)phenyl]ethynyl]phenyl)-1-[3-(4-cyclobutylpiperazine-1-yl)propyl]-1,4,6,7-tetrahydro-5H-pyrazolo[4,3-c]pyridine-5-carboxamide;
1-{3-[4-(Aminocarbonyl)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-{[4-({[(4-chlorophenyl)methyl]amino-4-methyl)phenyl]ethynyl]phenyl)-1-[3-(1,4-dioxa-8-azaspiro[4.5]dec-8-yl)propyl]-1,4,6,7-tetrahydro-5H-pyrazolo[4,3-c]pyridine-5-carboxamide;
3-(4-Chloro-3-[4-(3-[(4-chlorophenyl)methyl]amino]methyl)phenyl]ethynyl]phenyl)-1-[3-(4-cyclopropylpiperazin-1-yl)propyl]-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
1,1-Dimethylethyl 7-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-{4-[(2-Chloro-5-{5-(methylsulfonyl)-1-[3-(2-oxa-6-azaspiro[3.3]hept-6-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-2,3-dihydro-1H-inden-1-amine;
(2S)-1-(4,4′-Bipiperidin-1-yl)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]propan-2-ol;
(2S)-1-(4,4′-Bipiperidin-1-yl)-3-{3-[4-chloro-3-(cyclohexylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl}propan-2-ol;
3-(4-Chloro-3-{2-[4-({[(4-chlorophenyl)methyl]amino(methyl)phenyl]ethyl]phenyl)-1-(3-morpholin-4-ylpropyl)-1,4,6,7-tetrahydro-5H-pyrazolo[4,3-c]pyridine-5-carboxamide;
3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
1-[(2S)-3-(4,4′-Bipiperidin-1-yl)-2-hydroxypropyl]-3-{4-chloro-3-[2-(4-chlorophenyl)ethyl]phenyl}-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}methyl)-2,2,2-trifluoroethanamine;
7-{[2-Chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}-2-cyclopropyl-1,2,3,4-tetrahydroisoquinoline;
3-[4-Chloro-3-(1H-pyrrolo[2,3-b]pyridin-6-ylethynyl)phenyl]-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-[(4-methylpiperazin-1-yl)carbonyl]-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
3-(4-Chloro-3-{[4-({[(3-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(2-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-2-benzazepine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]-N-methylmethanamine;
1,1-Dimethylethyl (1S,4S)-5-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1,1-Dimethylethyl (1R,4R)-5-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1-[4-({2-Chloro-5-[1-{3-[(1S,4S)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(1R,4R)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
2-[3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridin-5-yl]-2-oxoacetamide;
1-[4-({2-Chloro-5-[5-(morpholin-4-ylcarbonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-carboxamide;
N-{2-[3-(4-Chloro-3-{[4-({[(4-chlorophenyl)methyl]amino-methyl)phenyl]ethynyl]phenyl)-1-(3-morpholin-4-ylpropyl)-1,4,6,7-tetrahydro-5H-pyrazolo[4,3-c]pyridin-5-yl]-2-oxoethylacetamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl-4-ethynyl)-2-(2,2,2-trifluoroethyl)-1,2,3,4-tetrahydroisoquinoline;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]-N-methylmethanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(cyclopropylmethyl)-1,2,3,4-tetrahydroisoquinoline;
2-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
3-(4-Chloro-3-{[4-([(3-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl]ethynyl)phenyl]methyl]amino)propan-1-ol;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydrofuran-2-ylmethyl)methanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}aniline;
6-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
3-(4-Chloro-3-[3-({[(2-chlorophenyl)methyl]amino-3-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(2-fluorophenyl)methyl]amino-3-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(4-fluorophenyl)methyl]amino-3-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-methyl-1,2,3,4-tetrahydroisoquinoline;
6-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenylaethynyl)phenyl]methyl}-2-methylpropan-1-amine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(2-methylpropyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methylsulfonyl)phenyl]methyl}methanamine;
1,1-Dimethylethyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1-oxo-3,4-dihydroisoquinoline-2(1H)-carboxylate;
Ethyl [6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinolin-2(1H)-yl]acetate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-prop-2-en-1-yl-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methyl-N-(phenylmethyl)methanamine;
(2R)-1-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-[(3S)-3-methylmorpholin-4-yl]propan-2-ol;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-methyl-N-(phenylmethyl)methanamine;
N-{[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenylethyl)phenyl]methyl}-2-methylpropan-1-amine;
3-(4-Chloro-3-{[4-([(2-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-({4-[({[2-(methyloxy)phenyl]methyl]amino)methy]phenyl]ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-(−4-[({[3-(methyloxy)phenyl]methyl]amino)methyl]phenyl(ethynyl)phenyl]-1-(3-thiomorpholin-4-ylpropyl)-1,4,6,7-tetrahydro-5H-pyrazolo[4,3-c]pyridine-5-carboxamide;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine;
1-[4-({2-Chloro-5-[1-{3-[(3R)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{5-(methylsulfonyl)-1-[3-(1-oxidothiomorpholin-4-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
3-[4-Chloro-3-({4-[({[4-(methyloxy)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
1-[5-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-fluorophenyl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(methyloxy)phenyl]-N-(phenylmethyl)methanamine;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}-ethynyl)-3-(morpholin-4-ylcarbonyl)-1,2,3,4-tetrahydroisoquinoline;
3-(4-Chloro-3-{[4-({[(2-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(3-fluorophenyl)methyl]amino-methyl)phenyl]ethynyl(phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(4-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-{[4-({[(4-chlorophenyl)methyl]amino-carbonyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(morpholin-2-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-({4-[(4R)-4-hydroxy-2-oxopyrrolidin-1-yl]piperidin-1-yl}methyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-[4-Chloro-3-({4-[({[4-(methylsulfanyl)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-(3-{[4-({[(4-Aminophenyl)methyl]amino]methyl)phenyl]ethynyl}-4-chlorophenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(3R)-3-hydroxypyrrolidin-1-yl]carbonyl}phenyl)ethynyl]phenyl}-1-(3-thiomorpholin-4-ylpropyl)-1,4,6,7-tetrahydro-5H-pyrazolo[4,3-c]pyridine-5-carboxamide;
N-[(2-Chloro-5-{[2-chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}phenyl)methyl]glycine;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(3-hydroxypropyl)benzamide;
3-[4-Chloro-3-({4-chloro-3-[(3-hydroxypropyl)carbamoyl]phenyl(ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(2S)-pyrrolidin-2-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(2S)-pyrrolidin-2-ylmethyl]benzamide;
3-{4-Chloro-3-[(4-chloro-3-[(2S)-pyrrolidin-2-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
tert-Butyl 3-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorobenzyl)oxy]pyrrolidine-1-carboxylate;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(3-hydroxypropyl)benzamide;
2-(3-{4-Chloro-3-[(4-chloro-3-{[(2-hydroxy-2-methylpropyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-[3-{4-Chloro-3-[(4-chloro-3-{[(2-hydroxy-2-methylpropyl)amino]methyl}phenyl)ethynyl]phenyl}-1-(3-thiomorpholin-4-ylpropyl)-1,4,6,7-tetrahydro-5H-pyrazolo[4,3-c]pyridin-5-yl]-2-oxoacetamide;
2-[3-(4-Chloro-3-{[4-chloro-3-({[(1R)-2-hydroxy-1-phenylethyl]amino}methyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(1R)-2-hydroxy-1-phenylethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[3-(methylamino)propyl]benzamide;
2-(3-{4-Chloro-3-({4-chloro-3-[(ethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
2-[3-(4-Chloro-3-{[4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
3-(4-Chloro-3-{[4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
2-[3-{[4-Chloro-3-{[(4-chloro-3-(pyrrolidin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)methylcarbamate;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)carbamate;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(3R)-pyrrolidin-3-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(piperidin-3-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(morpholin-2-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
3-{4-Chloro-3-[(4-chloro-3-[(3R)-pyrrolidin-3-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(piperidin-3-ylmethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(morpholin-2-ylmethyl)carbamoyl]phenyl(ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(3R)-pyrrolidin-3-ylmethyl]benzamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(piperidin-3-ylmethyl)benzamide;
2-[3-(4-Chloro-3-{[4-chloro-3-(morpholin-4-ylmethyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-({4-chloro-3-[(cyclopropylamino) methyl]phenylethynyl)phenyl]-1-{3-[(3S)-3-methylmorpholin-4-yl]propyl}-1,4,6,7-tetrahydro-5H-pyrazolo[4,3-c]pyridin-5-yl)-2-oxoacetamide;
3-[4-Chloro-3-({4-chloro-3-[(2-morpholin-4-ylethyl)carbamoyl]phenyl}ethynyl)phenyl]-1-(3-piperidin-1-ylpropyl)-1,4,6,7-tetrahydro-5H-pyrazolo[4,3-c]pyridine-5-carboxamide;
5-[(5-{5-[Amino(oxo)acetyl]-1-(3-piperidin-1-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
2-{3-[4-Chloro-3-({4-chloro-3-[(ethylamino) methyl]phenylethynyl)phenyl]-1-(3-piperidin-1-ylpropyl)-1,4,6,7-tetrahydro-5H-pyrazolo[4,3-c]pyridin-5-yl}-2-oxoacetamide;
2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]ethanamine;
2-{3-[4-Chloro-3-({4-chloro-3-[(cyclopentylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
3-[4-Chloro-3-(4-chloro-3-[(cyclopropylamino) methyl]phenylethynyl)phenyl]-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopentanamine;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(azetidin-3-ylmethyl)-2-chlorobenzamide;
3-[4-Chloro-3-({4-chloro-3-[(cyclopentylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
2-{3-[4-Chloro-3-({4-chloro-3-[(cyclopropylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
2-[3-{[4-Chloro-3-{[(4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridin-5-yl]-2-oxoacetamide;
3-(4-Chloro-3-{[4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopropanamine;
3-(4-Chloro-3-{[4-chloro-3-(morpholin-4-ylmethyl)phenyl]ethynyl]phenyl)-5-(methylsulfonyl)-1-(3-piperidin-1-ylpropyl)-4,5,6,7-tetrahydro-1H-pyrazolo[4,3-c]pyridine;
3-[4-Chloro-3-({4-chloro-3-[(cyclopropylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(morpholin-4-ylmethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-({4-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
3-{4-Chloro-3-[(4-{[(2S)-pyrrolidin-2-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-[(2S)-pyrrolidin-2-ylmethyl]benzamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[(2S)-pyrrolidin-2-ylmethyl]benzamide;
3-{4-Chloro-3-[(4-{[3-(methylamino)propyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-[3-(methylamino)propyl]benzamide; and
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[3-(methylamino)propyl]benzamide;
3-[4-Chloro-3-({4-chloro-3-[(dimethylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
2-(3-{4-Chloro-3-({4-chloro-3-[(dimethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N,N-dimethylmethanamine;
2-[3-{[4-Chloro-3-{[(4-(hydroxymethyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-[(4-chloro-3-{[(1-methylethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-(3-{4-Chloro-3-[(4-{[(4-chlorobenzyl)amino]methyl-phenyl)ethynyl]phenyl}-1-{3-[(3R)-3-methylmorpholin-4-yl]propyl}-1,4,6,7-tetrahydro-5H-pyrazolo[4,3-c]pyridin-5-yl)-2-oxoacetamide;
2-(3-{4-Chloro-3-[(4-{[(4-chlorobenzyl)amino]methyl[phenyl)ethynyl]phenyl}-1-[3-(3-methylmorpholin-4-yl)propyl]-1,4,6,7-tetrahydro-5H-pyrazolo[4,3-c]pyridin-5-yl)-2-oxoacetamide;
and pharmaceutically acceptable salts thereof.

59. A pharmaceutical composition according to claim 55, wherein said chemical entity is selected from the group consisting of:
4-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}butan-1-ol;
5-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}pentan-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}propan-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}propan-1-amine;
1-[1-(3-{3-[3-(3-Aminopropyl)-4-chlorophenyl]-5-(methylsulfonyl)-4,5,6,7-tetrahydro-1H-pyrazolo[4,3-c]pyridin-1-yl}propyl)piperidin-4-yl]pyrrolidin-2-one;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynylamine; and
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propylamine;
and pharmaceutically acceptable salts thereof.

60. A method of treating a subject suffering from or diagnosed with a disease, disorder, or medical condition mediated by cathepsin S activity, comprising administering to a subject in need of such treatment an effective amount of at least one chemical entity selected from compounds of Formula (I): 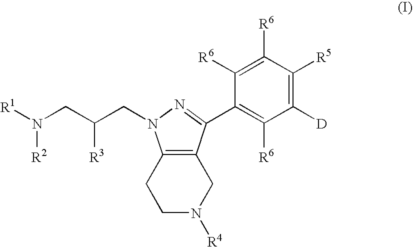
wherein:
R¹ and R² taken together with the nitrogen to which they are attached form a saturated monocyclic heterocycloalkyl group, each group optionally containing one additional heteroatom ring member that is O, S, or NR^{a}, and each group unsubstituted or substituted with one, two, or three R^{b} substituents;
where R^{a} is H, C₁₋₄alkyl, —COC₁₋₄alkyl, —CO(phenyl), or —CO₂C₁₋₄alkyl, or a monocyclic cycloalkyl ring, phenyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R^{b} substituent is independently:
i) OH, C₁₋₄alkyl, —C₁₋₄alkyl-OH, CF₃, —NR^{c}R^{d}, halo, —OC₁₋₄alkyl, —COC₁₋₄alkyl, —CO₂C₁₋₄alkyl, —CO₂H, or —CONR^{e}R^{f};
ii) a monocyclic heterocycloalkyl group unsubstituted or substituted with C₁₋₄alkyl, —COC₁₋₄alkyl, —CO₂C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group or monocyclic heteroaryl group, each group unsubstituted or substituted with C₁₋₄alkyl, OH, —OC₁₋₄alkyl, —NR^{c}R^{d}, or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo;
vi) two R^{b} substituents form a methylene or ethylene bridge; or
vii) two R^{b} substituents on adjacent carbons taken together with the carbons to which they are attached form a saturated monocyclic cycloalkyl group or saturated monocyclic heterocycloalkyl group, each group unsubstituted or substituted with C₁₋₄alkyl, OH, —OC₁₋₄alkyl, —NR^{c}R^{d}, or halo;
where R^{c} is H or C₁₋₄alkyl;
R^{d} is H, C₁₋₄alkyl, —COC₁₋₄alkyl, —COC₁₋₄alkyl-OH, —CO₂C₁₋₄alkyl, —CONR^{x}R^{y}, or —SO₂C₁₋₄alkyl;
where R^{x} and R^{y} are each independently H or C₁₋₄alkyl; and
R^{e} and R^{f} are each independently H or C₁₋₄alkyl;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, or —NR^{t}R^{u}; —COCF₃; —CO-(monocyclic heteroaryl); —CO—(C-linked monocyclic heterocycloalkyl); —CO-(phenyl); —SO₂C₁₋₄alkyl; —SO₂CF₃; —SO₂NR^{t}R^{u}; —CONR^{t}R^{u}; —COCO₂C₁₋₄alkyl; or —COCONR^{t}R^{u};
where R^{t} and R^{u} are each independently H, C₁₋₄alkyl, or —COC₁₋₄alkyl; or R^{t} and R^{u} taken together with the nitrogen to which they are attached form a monocyclic heterocycloalkyl ring;
R⁵ is halo or CF₃;
each R⁶ is independently H or F;
D is —C≡C—R⁷, —CH═CH—R³, —(CH₂)₂₋₃—R³, or —(CH₂)₃₋₅—R⁹;
where R⁷ is:
I) a C₁₋₄alkyl group unsubstituted or substituted with OH, —OC₁₋₄alkyl, —NR^{g}R^{h}, phenyl, or phenoxy, each phenyl or phenoxy being unsubstituted or substituted with C₁₋₄alkyl, OH, —OC₁₋₄alkyl, halo, or CF₃;
where R^{g} and R^{h} are each independently H, C₁₋₄alkyl, —COC₁₋₄alkyl, —COphenyl, —CO₂C₁₋₄alkyl, —SO₂C₁₋₄alkyl, or —SO₂-phenyl; or R^{g} and R^{h} taken together with the nitrogen to which they are attached form a monocyclic heterocycloalkyl group; or
II) a monocyclic cycloalkyl group, phenyl group, or monocyclic heteroaryl group, each group unsubstituted or substituted with one or two R^{k} substituents;
where R⁸ is a phenyl group or monocyclic heteroaryl group, each group unsubstituted or substituted with one or two R^{k} substituents;
where R⁹ is OH or —NRⁿR^{o};
where Rⁿ is H or C₁₋₄alkyl; and
R^{o} is H, C₁₋₄alkyl, monocyclic cycloalkyl, —COC₁₋₄alkyl, —COphenyl, —CO₂C₁₋₄alkyl, —SO₂C₁₋₄alkyl, —SO₂-phenyl, —SO₂-benzyl, or —SO₂NR^{p}R^{g}, each phenyl or benzyl group being unsubstituted or substituted with one or two R^{k} substituents;
where R^{p} and R^{g} are each independently H or C₁₋₄alkyl;
or Rⁿ and R^{o} taken together with the nitrogen to which they are attached form a monocyclic saturated heterocycloalkyl ring unsubstituted or substituted with C₁₋₄alkyl, OH, —OC₁₋₄alkyl, halo, or CF₃;
where each R^{k} substituent in D is independently:
a) a C₁₋₄alkyl group unsubstituted or substituted with OH, —OC₁₋₄alkyl, —OC₁₋₄alkyl-OH, halo, —CO₂C₁₋₄alkyl, CO₂H, CN, —NR^{r}R^{s}, —N(R^{r})COphenyl, —N(R^{r})SO₂C₁₋₄alkyl, —N(R^{r}) SO₂-phenyl, —SO₂C₁₋₄alkyl, phenyl, or phenoxy;
b) a substituent of formula 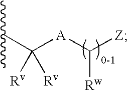
where each R^{v} is independently H or C₁₋₄alkyl, or both R^{v} substituents together form a carbonyl;
R^{w} is H, C₁₋₄alkyl, —CH₂OH, or —CO₂C₁₋₄alkyl;
A is O or NR^{aa};
where R^{aa} is H or C₁₋₄alkyl; and
Z is a phenyl group, benzyl group, cycloalkyl group, heterocycloalkyl group, heteroaryl group, or —CH₂— (heteroaryl) group, each group unsubstituted or substituted with one or two substituents independently selected from the group consisting of C₁₋₄alkyl, CF₃, halo, OH, —OC₁₋₄alkyl, —OCF₃, —OCHF₂, NR^{dd}R^{ee}, —CO₂C₁₋₄alkyl, —SC₁₋₄alkyl, and —SO₂C₁₋₄alkyl;
where R^{dd} and R^{ee} are each independently H or C₁₋₄alkyl;
c) two adjacent R^{k} substituents taken together with the carbons to which they are attached form a fused phenyl ring, monocyclic heteroaryl ring, monocyclic heterocycloalkyl ring, or monocyclic cycloalkyl ring, each fused ring unsubstituted or substituted with: C₁₋₄alkyl, —C₁₋₄alkyl-CF₃, —C₁₋₄alkyl-OH, —C₁₋₄alkyl-CO₂C₁₋₄alkyl, CF₃, C₂₋₄alkenyl, halo, OH, —OC₁₋₄alkyl, —COC₁₋₄alkyl, —COCF₃, —CO₂C₁₋₄alkyl, —CO₂H, —CONR^{ff}R^{gg}, or —SO₂C₁₋₄alkyl; or with a cycloalkyl group, —CH₂-(cycloalkyl) group, or benzyl group, each group unsubstituted or substituted with C₁₋₄alkyl, OH, —OC₁₋₄alkyl, halo, or CF₃;
where R^{ff} and R^{gg} are each independently H or C₁₋₄alkyl, or R^{ff} and R^{gg} taken together with the nitrogen to which they are attached form a monocyclic heterocycloalkyl ring, unsubstituted or substituted with C₁₋₄alkyl or OH; or
d) OH; —OC₁₋₄alkyl; halo; CF₃; —CHO; —CO₂C₁₋₄alkyl; CO₂H; CN; —NO₂; —CONR^{r}R^{s}, —NR^{r}R^{s}; —N(R^{r})_phenyl; —N(R^{r})-benzyl; —N(R^{r})-phenethyl; —N(R^{r})COC₁₋₄-alkyl; —N (R^{r}) CO-phenyl; —N(R^{r})SO₂C₁₋₄alkyl; —N (R^{r})SO₂-phenyl; —SO₂C₁₋₄alkyl; phenoxy; or a heteroaryl group; where each phenyl, benzyl, phenethyl, phenoxy, or heteroaryl group is unsubstituted or substituted with C₁₋₄alkyl, OH, —OC₁₋₄alkyl, halo, or CF₃;
where R^{r} is H, C₁₋₄alkyl, C₂₋₄alkyl-OH; and
R^{s} is H, C₁₋₄alkyl, —C₁₋₄alkyl-CF₃, —C₁₋₄alkyl-CN, —C₂₋₄alkyl-OH, —C₂₋₄alkyl-NR^{bb}R^{cc}, —C₁₋₄alkylCO₂C₁₋₄alkyl, —C₁₋₄alkylCO₂H, C₃₋₄alkenyl, —COC₁₋₄alkyl, or —CO₂C₁₋₄alkyl;
where R^{b} is H or C₁₋₄alkyl; and
R^{cc} is H, C₁₋₄alkyl, —COC₁₋₄alkyl, or —CO₂C₁₋₄alkyl;
or R^{bb} and R^{cc} taken together with the nitrogen to which they are attached from a monocyclic heterocycloalkyl ring;
or R^{r} and R^{s} taken together with the nitrogen to which they are attached form a heterocycloalkyl group unsubstituted or substituted with C₁₋₄alkyl, OH, —OC₁₋₄alkyl, halo, CF₃, or a monocyclic heterocycloalkyl ring unsubstituted or substituted with OH;
and pharmaceutically acceptable salts, prodrugs, and metabolites thereof.

61. A method according to claim 60, wherein said chemical entity is selected from the group consisting of:
3-[4-Chloro-3-(1H-indol-5-ylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[2-(1H-indol-5-yl)-ethyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
4-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-butyric acid;
3-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-propionic acid;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
3-(4-Chloro-3-thiophen-2-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3,4-dichloro-phenylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[4-(4-iodo-phenoxy)-phenylethynyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
3-[4-Chloro-3-(4-phenoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[3-(4-Bromo-phenylethynyl)-4-chloro-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzoic acid;
3-(4-Chloro-3-pyridin-4-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-3-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-2-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-thiophen-3-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
3-[4-Chloro-3-(4-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-p-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(4-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2,4-difluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-o-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzaldehyde;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenylamine;
3-(4-Chloro-3-phenylethynyl-phenyl)-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-carbamic acid tert-butyl ester;
3-[4-Chloro-3-(3-phenyl-prop-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-phenyl-but-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(5-phenyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-yn-1-ol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-but-3-yn-1-ol;
5-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-pent-4-yn-1-ol;
3-(4-Chloro-3-hex-1-ynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-cyclohexylethynyl-phenyl)-5-methanesulfonyl-1-(3-morpholin-4-yl-propyl)-4,5,6,7-tetrahydro-1H-pyrazolo[4,3-c]pyridine;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-diethyl-amine;
3-{4-Chloro-3-[3-(1,1-dioxo-1λ⁶-thiomorpholin-4-yl)-prop-1-ynyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(4-methyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-phenoxy-prop-1-ynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzenesulfonamide;
3-{4-Chloro-3-[2-(4-phenoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenol;
3-{4-Chloro-3-[2-(4-chloro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-{4-Chloro-3-[2-(4-methoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-{4-Chloro-3-[2-(2,4-difluoro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(2-o-tolyl-ethyl)-phenyl]-5-methanesulfonyl-1-(3-morpholin-4-yl-propyl)-4,5,6,7-tetrahydro-1H-pyrazolo[4,3-c]pyridine;
[4-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenyl]-methanol;
3-(4-Chloro-3-phenethyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-phenyl-propyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetamide;
3-(4-Chloro-3-Z-styryl-phenyl)-5-methanesulfonyl-1-(3-morpholin-4-yl-propyl)-4,5,6,7-tetrahydro-1H-pyrazolo[4,3-c]pyridine;
3-(4-Chloro-3-E-styryl-phenyl)-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzylamine;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methanesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-acetamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzamide;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-(4-methyl-benzyl)-amine;
(4-Chloro-benzyl)-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methyl-amine;
3-[4-Chloro-3-(4-pyrrolidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-piperidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-methanesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-C-phenyl-methanesulfonamide;
3,4-Dichloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
4-Chloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methyl-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methoxy-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-N,N-dimethyl-sulfamoylurea;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-acetamide;
2-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propylsulfamoyl}-benzoic acid;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-nitro-benzenesulfonamide;
3-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-4-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methoxy-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methyl-benzenesulfonamide;
2-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-nitro-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-cyano-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methanesulfonyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methanesulfonyl-benzenesulfonamide;
1-[1-(3-{3-[4-Chloro-3-(3-pyrrolidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-piperidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(3-methyl-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[3-(4,4-difluoro-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(3-morpholin-4-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(4-methyl-piperazin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(3-Azepan-1-yl-propyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-cyclopentylamino-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-pyrrolidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-piperidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[4-(3-methyl-piperidin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[4-(4-methyl-piperazin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(4-morpholin-4-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Azepan-1-yl-butyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-cyclopentylamino-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
8-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-2,8-diaza-spiro[4.5]decan-1-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid amide;
3-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-1,3-dihydro-imidazo[4,5-b]pyridin-2-one;
[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
1-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-3-morpholin-4-yl-propan-2-ol;
2-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-cyclopentanone;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-morpholin-4-yl-propan-2-ol;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperidin-1-yl-propan-2-ol;
3-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-,3-dihydro-imidazo[4,5-b]pyridin-2-one;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid amide;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-pyrrolidin-1-yl-propan-2-ol;
[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
4-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperazine-1-carboxylic acid tert-butyl ester;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperazin-1-yl-propan-2-ol;
1-(4-Amino-piperidin-1-yl)-3-{3-[4-chloro-3-(4-chloro-phenylethynyl)-phenyl]-5-methanesulfonyl-4,5,6,7-tetrahydro-pyrazolo[4,3-c]pyridin-1-yl}-propan-2-ol;
1-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-3-piperidin-1-yl-propan-2-ol;
1-(3-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-propyl)-piperidine-4-carboxylic acid amide;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-oxo-acetic acid methyl ester;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-pyridin-2-yl-methanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-furan-2-yl-methanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2,2,2-trifluoro-ethanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-fluoro-ethanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-(tetrahydro-furan-2-yl)-methanone;
Acetic acid 2-[3-[4-chloro-3-(4-chloro-phenylethynyl)-phenyl]-1-(3-morpholin-4-yl-propyl)-1,4,6,7-tetrahydro-pyrazolo[4,3-c]pyridin-5-yl]-2-oxo-ethyl ester;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-hydroxy-ethanone;
3-[4-Chloro-3-(4-chloro-phenylethynyl)-phenyl]-1-(3-morpholin-4-yl-propyl)-1,4,6,7-tetrahydro-pyrazolo[4,3-c]pyridine-5-carboxylic acid amide;
3-{4-Chloro-3-[2-(4-chloro-phenyl)-ethyl]-phenyl}-1-(3-morpholin-4-yl-propyl)-1,4,6,7-tetrahydro-pyrazolo[4,3-c]pyridine-5-carboxylic acid amide;
1-{1-[3-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-pyrrolidin-2-one;
1-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-3-morpholin-4-yl-propan-2-ol;
8-[3-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-2-hydroxy-propyl]-2,8-diaza-spiro[4.5]decan-1-one; and
1-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-3-morpholin-4-yl-propan-2-ol;
and pharmaceutically acceptable salts thereof.

62. A method according to claim 60, wherein said chemical entity is selected from the group consisting of:
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoacetamide;
3-(4-Chloro-3-[4-({[(4-chlorophenyl)methyl]aminomethyl)phenyl]ethynyl]phenyl)-1-(3-morpholin-4-ylpropyl)-1,4,6,7-tetrahydro-5H-pyrazolo[4,3-c]pyridine-5-carboxamide;
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-N,N-dimethyl-2-oxoacetamide;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-sulfonamide;
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoethanamine;
3-(4-Chloro-3-{[4-({[(4-chlorophenyl)methyl]amino]methyl)phenyl]ethynyl]phenyl)-N-methyl-1-(3-morpholin-4-ylpropyl)-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-pyridin-2-ylpiperazin-1-yl)propyl]-1,4,6,7-tetrahydro-5H-pyrazolo[4,3-c]pyridine-5-carboxamide;
2-(3-{4-Chloro-3-[(4-[(4-chlorobenzyl)amino]methylphenyl)ethynyl]phenyl}-1-{3-[(3S,5S)-3,5-dimethylmorpholin-4-yl]propyl}-1,4,6,7-tetrahydro-5H-pyrazolo[4,3-c]pyridin-5-yl)-2-oxoacetamide;
2-[3-{[4-Chloro-3-{[(4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-{3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridin-5-yl}-2-oxoacetamide;
3-(4-Chloro-3-{[4-({[(4-chlorophenyl)methyl]amino(methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(pyridin-3-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-{[(pyridin-3-ylmethyl)amino]carbonyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-{[(piperidin-4-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[4-(2-oxopyrrolidin-1-yl)piperidin-1-yl]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
1-[4-({2-Chloro-5-[1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-({4-chloro-3-[(ethylamino)methyl]phenyl}ethynyl)phenyl]-1-(3-piperidin-1-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(2-morpholin-4-ylethyl)benzamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(2-morpholin-4-ylethyl)benzamide;
3-{4-Chloro-3-[(4-{[(4-chlorobenzyl)amino]methyl(phenyl)ethynyl]phenyl}-1-{3-[(3aR,6aS)-tetrahydro-1H-furo[3,4-c]pyrrol-5(3H)-yl]propyl}-1,4,6,7-tetrahydro-5H-pyrazolo[4,3-c]pyridine-5-carboxamide;
3-(4-Chloro-3-{[3-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoethanol;
3-(4-Chloro-3-{[4-([(4-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(1-methylethyl)oxy]phenyl}methyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-({4-[({[4-(dimethylamino)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(difluoromethyl)oxy]phenyl]methyl)amino]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
1-{4-[(2-Chloro-5-{5-(methylsulfonyl)-1-[3-(4-pyridin-2-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperidin-1-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-[4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)benzaldehyde;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydro-2H-pyran-4-ylmethyl)methanamine;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(tetrahydro-2H-pyran-4-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
Methyl N-{[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycine;
2-(1,1-Dimethylethyl) 3-methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-methyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N,N-dimethyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
Methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
Methyl (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
2-(1,1-Dimethylethyl) 3-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
2-(1,1-Dimethylethyl) 1-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-1,2(1H)-dicarboxylate;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-1-carboxylate;
(2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
Methyl (2R)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3,4-dichlorophenyl)methyl]methanamine;
(1S,2R)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl]phenyl)ethynyl]phenyl}methyl)amino]-2,3-dihydro-1H-inden-1-ol;
(1R,2S)-1-[({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)amino]-2,3-dihydro-1H-inden-2-ol;
(1R)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1S)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1R)—N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(2S)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)amino]-2-phenylethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
Methyl N-{[5-({5-[5-(aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl}ethynyl)-2-chlorophenyl]methyl}glycinate;
N-{[5-({5-[5-(Aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl]ethynyl)-2-chlorophenyl]methyl]glycine;
3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridine-5-carboxylic acid amide;
3-{4-Chloro-3-[(4-chloro-3-{[(tetrahydrofuran-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
3-{4-Chloro-3-[(4-chloro-3-{[(phenylmethyl)amino]methyl(phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-methyl-1,2,3,4-tetrahydroisoquinoline;
N-[1-(3-{3-[4-Chloro-3-(1,2,3,4-tetrahydroisoquinolin-7-ylethynyl)phenyl]-5-(methylsulfonyl)-4,5,6,7-tetrahydro-1H-pyrazolo[4,3-c]pyridin-1-yl}propyl)piperidin-4-yl]acetamide;
7-(2-{2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethyl)-1,2,3,4-tetrahydroisoquinoline;
7-[2-(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethyl]-1,2,3,4-tetrahydroisoquinoline;
1,1-Dimethylethyl 1′-{(2S)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
1,1-Dimethylethyl 1′-[(2S)-3-[3-[4-chloro-3-(4-methylpent-1-yn-1-yl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(cyclohexylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-(2S)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]-2-hydroxypropyl-4,4′-bipiperidine-1-carboxylate;
1,1-Dimethylethyl 1′-{(2S)-3-[5-(aminocarbonyl)-3-{4-chloro-3-[(4-chlorophenyl)ethynyl]phenyl}-4,5,6,7-tetrahydro-1H-pyrazolo[4,3-c]pyridin-1-yl]-2-hydroxypropyl}-4,4′-bipiperidine-1-carboxylate;
1-[(2S)-3-(4,4′-Bipiperidin-1-yl)-2-hydroxypropyl]-3-{4-chloro-3-[(4-chlorophenyl)ethynyl]phenyl}-1,4,6,7-tetrahydro-5H-pyrazolo[4,3-c]pyridine-5-carboxamide;
(2S)-1-(4,4′-Bipiperidin-1-yl)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl}propan-2-ol;
(2S)-1-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(2S)-1-(1′-Acetyl-4,4′-bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
1,1-Dimethylethyl 1′-{(2S)-3-[5-(aminocarbonyl)-3-{4-chloro-3-[2-(4-chlorophenyl)ethyl]phenyl}-4,5,6,7-tetrahydro-1H-pyrazolo[4,3-c]pyridin-1-yl]-2-hydroxypropyl}-4,4′-bipiperidine-1-carboxylate;
2-[3-(4-Chloro-3-{2-[4-({[(4-chlorophenyl)methyl]amino]methyl)phenyl]ethyl]phenyl)-{3-[(3S)-3-methylmorpholin-4-yl]propyl}-1,4,6,7-tetrahydro-5H-pyrazolo[4,3-c]pyridin-5-yl]-2-oxoacetamide;
3-(4-Chloro-3-{2-[4-({[(4-chlorophenyl)methyl]amino]methyl)phenyl]ethyl]phenyl)-{3-[(3S)-3-methylmorpholin-4-yl]propyl}-1,4,6,7-tetrahydro-5H-pyrazolo[4,3-c]pyridine-5-carboxamide;
1,1-Dimethylethyl 1′-{(2S)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
(2S)-1-(4,4′-Bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-(1′-Acetyl-4,4′-bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-[3-{4-Chloro-3-[2-(4-chlorophenyl)ethyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(S)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(S)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(S)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(2-{3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
2-{3-(4-Chloro-3-{4-[(4-chloro-benzylamino)-methyl]-phenylethynyl}-phenyl)-1-[(2R)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
(S)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
2-{3-[4-Chloro-3-(4-chloro-3-{[((2S)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
2-{3-[4-Chloro-3-(4-chloro-3-{[((2R)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-cyclopentylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-propylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[3-(3-{[Bis-(2-hydroxy-ethyl)-amino]-methyl)-4-chloro-phenylethynyl)-4-chloro-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-5-(5-{5-Aminooxalyl-1-[3-(3-methyl-morpholin-4-yl)-propyl]-4,5,6,7-tetrahydro-1H-pyrazolo[4,3-c]pyridin-3-yl}-2-chloro-phenylethynyl)-2-chloro-N-(3-methylamino-propyl)-benzamide;
(S)-2-{3-{4-Chloro-3-[4-chloro-3-(3-hydroxy-propoxymethyl)-phenylethynyl]-phenyl}-1-[3-(3-methyl-morpholin-4-yl)-propyl]-1,4,6,7-tetrahydro-pyrazolo[4,3-c]pyridin-5-yl}-2-oxo-acetamide;
(S)-2-{3-{4-Chloro-3-[4-chloro-3-(pyrrolidin-3-yloxymethyl)-phenylethynyl]-phenyl}-1-[3-(3-methyl-morpholin-4-yl)-propyl]-1,4,6,7-tetrahydro-pyrazolo[4,3-c]pyridin-5-yl}-2-oxo-acetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-(1-methylethyl)-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
2-(3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
2-(3-{4-Chloro-3-[(4-chloro-3-{[(phenylmethyl)amino]methyl{phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-(2-{2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine; 2-(3-{4-Chloro-3-[(4-chloro-3-{[(pyridin-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-(phenylmethyl)methanamine;
3-{4-Chloro-3-[(4-chloro-3-[(phenylmethyl)amino]methyl(phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-chloro-3-[(pyridin-2-ylmethyl)amino]methyl(phenyl)ethynyl]phenyl}-1-(3-thiomorpholin-4-ylpropyl)-1,4,6,7-tetrahydro-5H-pyrazolo[4,3-c]pyridine-5-carboxamide;
2-({[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]methyl]amino)ethanol;
Methyl N-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)methyl]glycinate; and
2-(3-{4-Chloro-3-[(4-chloro-3-{[(2-hydroxyethyl)amino]methylphenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
and pharmaceutically acceptable salts thereof.

63. A method according to claim 60, wherein said chemical entity is selected from the group consisting of:
3-(2-{3-[5-(Methylsulfonyl)-1-(3-morpholin-4-ylpropyl)-4,5,6,7-tetrahydro-1H-pyrazolo[4,3-c]pyridin-3-yl]phenyl}ethyl)phenol;
3-{4-Chloro-3-[(4-chlorophenyl)ethynyl]phenyl}-1-(3-morpholin-4-ylpropyl)-4,5,6,7-tetrahydro-1H-pyrazolo[4,3-c]pyridine;
8-{3-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-2,8-diazaspiro[4.5]decan-1-one;
4-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}but-3-yn-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}prop-2-yn-1-amine;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl}prop-2-yn-1-yl)benzenesulfonamide;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl]propyl)benzenesulfonamide;
1-[1-(3-{3-[3-(3-Aminoprop-1-yn-1-yl)-4-chlorophenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl 2-{[(3-{2-chloro-5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]phenyl}propyl)amino]sulfonyl}benzoate;
1-[1-(3-{3-[4-Chloro-3-(3-hydroxyprop-1-yn-1-yl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{3-[4-Chloro-3-(4-hydroxybutyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-[4-Chloro-3-[4-(dimethylamino)butyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[1-(3-{3-[4-Chloro-3-(3-hydroxypropyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-[4-Chloro-3-[3-(dimethylamino)propyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methylmethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-N-ethylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenylaethynyl)phenyl]methyl}-2-methylpropan-1-amine;
1-[4-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-[(4-chlorophenyl)methyl]methanamine;
3-(3-{[4-(1H-Benzimidazol-2-yl)phenyl]ethynyl]-4-chlorophenyl)-5-(methylsulfonyl)-1-(3-morpholin-4-ylpropyl)-4,5,6,7-tetrahydro-1H-pyrazolo[4,3-c]pyridine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(phenylmethyl)aniline; {[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]amino]acetonitrile;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclopropanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclobutanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclopentanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclohexanamine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(2-phenylethyl)aniline;
1-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1,1-Dimethylethyl (1-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}piperidin-4-yl)carbamate;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-ol;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-amine;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-morpholin-4-ylpropan-2-ol;
1-[4-({2-Chloro-5-[5-(methylsulfonyl)-1-(3-pyrrolidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
Ethyl 1-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxylate;
1-{4-[(2-Chloro-5-{1-[3-(1,4-dioxa-8-azaspiro[4.5]dec-8-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxylic acid;
(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanol; 1′-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-1,4′-bipiperidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methyloxy)phenyl]methyl}methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2,2,2-trifluoroethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(cyclopropylmethyl)methanamine;
(2S)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
1-{4-[(2-Chloro-5-{5-(methylsulfonyl)-1-[3-(4-morpholin-4-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4-methylpiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1-[4-({2-Chloro-5-[5-(methylsulfonyl)-1-{3-[4-(trifluoromethyl)piperidin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)acetamide;
Methyl N-{[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
1-(1-{3-[3-{4-Chloro-3-[(4-{[(2,2,2-trifluoroethyl)amino]methyl]phenyl)ethynyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}prop-2-en-1-amine;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxamide;
Methyl (2S)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-(1-{3-[3-(4-Chloro-3-{[4-({[(1R)-2-hydroxy-1-phenylethyl]aminomethyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1-{4-[(5-{1-[3-(4-Acetylpiperazin-1-yl)propyl]-5-(methylsulfonyl)-4,5,6,7-tetrahydro-1H-pyrazolo[4,3-c]pyridin-3-yl}-2-chlorophenyl)ethynyl]phenyl}-N-[(4-chlorophenyl)methyl]methanamine;
1-{4-[(2-Chloro-5-{1-[3-(4-methylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4,4-dimethylpiperidin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)-2-hydroxyacetamide;
1-{4-[(2-Chloro-5-{1-[3-(4,4-difluoropiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1-{4-[(2-Chloro-5-{1-[3-(4-fluoropiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanesulfonamide;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-piperidin-1-ylpropan-2-ol;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidin-4-yl)acetamide;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidine-4-carboxamide;
3-(4-Chloro-3-{[2-(trifluoroacetyl)-2,3-dihydro-1H-isoindol-5-yl]ethynyl}phenyl)-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1,1-Dimethylethyl 4-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}piperazine-1-carboxylate;
1-[4-({2-Chloro-5-[5-(methylsulfonyl)-1-(3-piperazin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-4,5,6,7-tetrahydro-1H-pyrazolo[4,3-c]pyridin-1-yl]propyl}piperidin-4-yl)acetamide;
1,1-Dimethylethyl 7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl)ethynyl)-3,4-dihydroisoquinoline-2(1H)-carboxylate;
1,1-Dimethylethyl 7-({2-chloro-5-[1-(2-hydroxy-3-piperidin-1-ylpropyl)-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-[4-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1,1-Dimethylethyl 7-({5-[1-{3-[4-(aminocarbonyl)piperidin-1-yl]-2-hydroxypropyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
1,1-Dimethylethyl 7-({5-[1-{3-[4-(aminocarbonyl)piperidin-1-yl]propyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-3-benzazepine;
1,1-Dimethylethyl {[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
7-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[5-(methylsulfonyl)-1-{3-[4-(phenylcarbonyl)piperazin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
7-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-1,2,3,4-tetrahydroisoquinoline;
(3S)-1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
(3R)-1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
1,1-Dimethylethyl {[2-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[1-{3-[(2R,6S)-2,6-dimethylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
(4-{3-[3-(4-Chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}morpholin-2-yl)methanol;
1,1-Dimethylethyl 4-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-1,4-diazepane-1-carboxylate;
1-{4-[(2-Chloro-5-{[1-[3-(1,4-diazepan-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1,1-Dimethylethyl 5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,3-dihydro-2H-isoindole-2-carboxylate;
3-[4-Chloro-3-(2,3-dihydro-1H-isoindol-5-ylethynyl)phenyl]-5-(methylsulfonyl)-1-(3-morpholin-4-ylpropyl)-4,5,6,7-tetrahydro-1H-pyrazolo[4,3-c]pyridine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro-1,6-naphthyridine;
1-{4-[2-(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethyl]phenyl}-N-[(4-chlorophenyl)methyl]methanamine;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-2-one;
1-{4-[(2-Chloro-5-{1-[3-(1,1-dioxidothiomorpholin-4-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(1,4-oxazepan-4-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-(4-{[5-(5-Acetyl-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}phenyl)-N-[(4-chlorophenyl)methyl]methanamine;
3-(4-Chloro-3-{[4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridine-5-carboxamide;
2-[3-(4-Chloro-3-{[4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoethanol;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro[1,2,4]triazolo[4,3-a]pyrazine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-3-methylpiperazin-2-one;
2-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
3-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
4-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
3-[3-(1H-Benzimidazol-5-ylethynyl)-4-chlorophenyl]-5-(methylsulfonyl)-1-(3-morpholin-4-ylpropyl)-4,5,6,7-tetrahydro-1H-pyrazolo[4,3-c]pyridine;
1-[4-({5-[5-Acetyl-1-(3-thiomorpholin-4-ylpropyl)-4,5,6,7-tetrahydro-1H-pyrazolo[4,3-c]pyridin-3-yl]-2-chlorophenyl]ethynyl)phenyl]-N-[(4-chlorophenyl)methyl]methanamine;
2-[3-(4-Chloro-3-{[4-({[(4-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridin-5-yl]-2-oxoethanol;
2-[3-{[4-Chloro-3-{[(4-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridin-5-yl]-2-oxoacetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-1,2,3,4-tetrahydroisoquinoline;
1-{4-[(2-Chloro-5-{5-(methylsulfonyl)-1-[3-(4-pyridin-4-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-pyridin-3-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
Methyl 4-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylate;
(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholin-3-yl)methanol;
1-[4-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(3-thienylmethyl)methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-(2-thienyl)ethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[5-methyl-2-(trifluoromethyl)furan-3-yl]methyl}methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-[(4-chlorophenyl)methyl]methanamine;
2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoacetamide;
3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridine-5-carboxamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(3-thienylmethyl)methanamine;
2-[3-(4-Chloro-3-{[4-({[(4-chlorophenyl)methyl]amino{methyl)phenyl]ethynyl]phenyl)-1-{3-[(1S,4S)-2-oxa-5-azabicyclo[2.2.1]hept-5-yl]propyl}-1,4,6,7-tetrahydro-5H-pyrazolo[4,3-c]pyridin-5-yl]-2-oxoacetamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-[(4-chlorophenyl)methyl]methanamine;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylic acid;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-(1H-imidazol-4-ylethynyl)phenyl]-5-(methylsulfonyl)-1-(3-morpholin-4-ylpropyl)-4,5,6,7-tetrahydro-1H-pyrazolo[4,3-c]pyridine;
2-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydroisoquinoline;
3-(4-Chloro-3-{[4-(1,3-dihydro-2H-isoindol-2-ylmethyl)phenyl]ethynyl}phenyl)-5-(methylsulfonyl)-1-(3-morpholin-4-ylpropyl)-4,5,6,7-tetrahydro-1H-pyrazolo[4,3-c]pyridine;
(1R)—N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydronaphthalen-1-amine;
(1S,2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2,3-dihydro-1H-inden-1-ol;
1-{3-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{3-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
(2S)-1-(4,4′-Bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
2-[3-{[4-Chloro-3-{[(4-({[(4-chlorophenyl)methyl]amino(methyl)phenyl]ethynyl}-phenyl)-1-(3-piperidin-1-ylpropyl)-1,4,6,7-tetrahydro-5H-pyrazolo[4,3-c]pyridin-5-yl]-2-oxoacetamide;
1-(3-{5-[Amino(oxo)acetyl]-3-(4-chloro-3-{[4-({[(4-chlorophenyl)methyl]amino]methyl)phenyl]ethynylphenyl)-4,5,6,7-tetrahydro-1H-pyrazolo[4,3-c]pyridin-1-yl}propyl)piperidine-4-carboxamide;
2-{3-(4-Chloro-3-{[4-({[(4-chlorophenyl)methyl]amino-4-methyl)phenyl]ethynyl]phenyl)-1-[3-(1,4-dioxa-8-azaspiro[4.5]dec-8-yl)propyl]-1,4,6,7-tetrahydro-5H-pyrazolo[4,3-c]pyridin-5-yl}-2-oxoacetamide;
2-[3-(4-Chloro-3-{[4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridin-5-yl]-2-oxoacetamide;
3-(4-Chloro-3-{[4-({[(4-chlorophenyl)methyl]amino(methyl)phenyl]ethynyl}-phenyl)-1-(3-pyrrolidin-1-ylpropyl)-1,4,6,7-tetrahydro-5H-pyrazolo[4,3-c]pyridine-5-carboxamide;
3-(4-Chloro-3-[4-({[(4-chlorophenyl)methyl]amino(methyl)phenyl]ethynyl]phenyl)-1-{3-[4-(1-oxidopyridin-2-yl)piperazin-1-yl]propyl}-1,4,6,7-tetrahydro-5H-pyrazolo[4,3-c]pyridine-5-carboxamide;
3-(4-Chloro-3-[4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridine-5-carboxamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(1,3-thiazol-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(1-methyl-1H-imidazol-5-yl)methyl]methanamine;
3-(4-Chloro-3-{[4-({[(4-chlorophenyl)methyl]amino(methyl)phenyl]ethynyl]phenyl)-1-(3-piperidin-1-ylpropyl)-1,4,6,7-tetrahydro-5H-pyrazolo[4,3-c]pyridine-5-carboxamide;
1-{3-[4-(Acetylamino)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-[4-(3-[(4-chlorophenyl)methyl]amino]methyl)phenyl]ethynyl]phenyl)-1-[3-(4-cyclobutylpiperazin-1-yl)propyl]-1,4,6,7-tetrahydro-5H-pyrazolo[4,3-c]pyridine-5-carboxamide;
1-{3-[4-(Aminocarbonyl)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-{[4-({[(4-chlorophenyl)methyl]amino-4-methyl)phenyl]ethynyl]phenyl)-1-[3-(1,4-dioxa-8-azaspiro[4.5]dec-8-yl)propyl]-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-cyclopropylpiperazin-1-yl)propyl]-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
1,1-Dimethylethyl 7-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-{4-[(2-Chloro-5-{5-(methylsulfonyl)-1-[3-(2-oxa-6-azaspiro[3.3]hept-6-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-2,3-dihydro-1H-inden-1-amine;
(2S)-1-(4,4′-Bipiperidin-1-yl)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]propan-2-ol;
(2S)-1-(4,4′-Bipiperidin-1-yl)-3-{3-[4-chloro-3-(cyclohexylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl}propan-2-ol;
3-(4-Chloro-3-{2-[4-({[(4-chlorophenyl)methyl]amino(methyl)phenyl]ethyl}phenyl)-1-(3-morpholin-4-ylpropyl)-1,4,6,7-tetrahydro-5H-pyrazolo[4,3-c]pyridine-5-carboxamide;
3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
1-[(2S)-3-(4,4′-Bipiperidin-1-yl)-2-hydroxypropyl]-3-{4-chloro-3-[2-(4-chlorophenyl)ethyl]phenyl}-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}methyl)-2,2,2-trifluoroethanamine;
7-{[2-Chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}-2-cyclopropyl-1,2,3,4-tetrahydroisoquinoline;
3-[4-Chloro-3-(1H-pyrrolo[2,3-b]pyridin-6-ylethynyl)phenyl]-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-[(4-methylpiperazin-1-yl)carbonyl]-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
3-(4-Chloro-3-{[4-({[(3-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(2-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-2-benzazepine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]-N-methylmethanamine;
1,1-Dimethylethyl (1S,4S)-5-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1,1-Dimethylethyl (1R,4R)-5-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1-[4-({2-Chloro-5-[1-{3-[(1S,4S)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(1R,4R)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
2-[3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridin-5-yl]-2-oxoacetamide;
1-[4-({2-Chloro-5-[5-(morpholin-4-ylcarbonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-carboxamide;
N-{2-[3-(4-Chloro-3-{[4-({[(4-chlorophenyl)methyl]amino-methyl)phenyl]ethynyl]phenyl)-1-(3-morpholin-4-ylpropyl)-1,4,6,7-tetrahydro-5H-pyrazolo[4,3-c]pyridin-5-yl]-2-oxoethylacetamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl-4-ethynyl)-2-(2,2,2-trifluoroethyl)-1,2,3,4-tetrahydroisoquinoline;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]-N-methylmethanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(cyclopropylmethyl)-1,2,3,4-tetrahydroisoquinoline;
2-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
3-(4-Chloro-3-{[4-([(3-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)propan-1-ol;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydrofuran-2-ylmethyl)methanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}aniline;
6-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
3-(4-Chloro-3-{[3-({[(2-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(2-fluorophenyl)methyl]amino-3-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(4-fluorophenyl)methyl]amino-3-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-methyl-1,2,3,4-tetrahydroisoquinoline;
6-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenylaethynyl)phenyl]methyl}-2-methylpropan-1-amine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(2-methylpropyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methylsulfonyl)phenyl]methyl}methanamine;
1,1-Dimethylethyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1-oxo-3,4-dihydroisoquinoline-2(1H)-carboxylate;
Ethyl [6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinolin-2(1H)-yl]acetate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-prop-2-en-1-yl-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methyl-N-(phenylmethyl)methanamine;
(2R)-1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-[(3S)-3-methylmorpholin-4-yl]propan-2-ol;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-methyl-N-(phenylmethyl)methanamine;
N-{[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenylethyl)phenyl]methyl}-2-methylpropan-1-amine;
3-(4-Chloro-3-{[4-([(2-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-{(4-[({[2-(methyloxy)phenyl]methyl]amino)methy]phenyl]ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-(−4-[({[3-(methyloxy)phenyl]methyl]amino)methyl]phenyl(ethynyl)phenyl]-1-(3-thiomorpholin-4-ylpropyl)-1,4,6,7-tetrahydro-5H-pyrazolo[4,3-c]pyridine-5-carboxamide;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine;
1-[4-({2-Chloro-5-[1-{3-[(3R)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{5-(methylsulfonyl)-1-[3-(1-oxidothiomorpholin-4-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
3-[4-Chloro-3-{(4-[({[4-(methyloxy)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
1-[5-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-fluorophenyl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(methyloxy)phenyl]-N-(phenylmethyl)methanamine;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(morpholin-4-ylcarbonyl)-1,2,3,4-tetrahydroisoquinoline;
3-(4-Chloro-3-{[4-({[(2-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-[4-({[(3-fluorophenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(4-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-{[4-([(4-chlorophenyl)methyl]aminocarbonyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(morpholin-2-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-({4-[(4R)-4-hydroxy-2-oxopyrrolidin-1-yl]piperidin-1-yl}methyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-[4-Chloro-3-({4-[({[4-(methylsulfanyl)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-(3-{[4-({[(4-Aminophenyl)methyl]amino]methyl)phenyl]ethynyl}-4-chlorophenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(3R)-3-hydroxypyrrolidin-1-yl]carbonyl}phenyl)ethynyl]phenyl}-1-(3-thiomorpholin-4-ylpropyl)-1,4,6,7-tetrahydro-5H-pyrazolo[4,3-c]pyridine-5-carboxamide;
N-[(2-Chloro-5-{[2-chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}phenyl)methyl]glycine;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(3-hydroxypropyl)benzamide;
3-[4-Chloro-3-({4-chloro-3-[(3-hydroxypropyl)carbamoyl]phenyl(ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(2S)-pyrrolidin-2-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(2S)-pyrrolidin-2-ylmethyl]benzamide;
3-{4-Chloro-3-[(4-chloro-3-[(2S)-pyrrolidin-2-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
tert-Butyl 3-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorobenzyl)oxy]pyrrolidine-1-carboxylate;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl-2-chloro-N-(3-hydroxypropyl)benzamide;
2-(3-{4-Chloro-3-[(4-chloro-3-{[(2-hydroxy-2-methylpropyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-[3-{4-Chloro-3-[(4-chloro-3-{[(2-hydroxy-2-methylpropyl)amino]methyl}phenyl)ethynyl]phenyl}-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-({[(1R)-2-hydroxy-1-phenylethyl]amino}methyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(1R)-2-hydroxy-1-phenylethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[3-(methylamino)propyl]benzamide;
2-(3-{4-Chloro-3-({4-chloro-3-[(ethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
2-[3-{[4-Chloro-3-{[(4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
3-(4-Chloro-3-{[4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
2-[3-{[4-Chloro-3-{[(4-chloro-3-(pyrrolidin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)methylcarbamate;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)carbamate;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(3R)-pyrrolidin-3-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(piperidin-3-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(morpholin-2-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
3-{4-Chloro-3-[(4-chloro-3-[(3R)-pyrrolidin-3-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(piperidin-3-ylmethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(morpholin-2-ylmethyl)carbamoyl]phenyl(ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(3R)-pyrrolidin-3-ylmethyl]benzamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(piperidin-3-ylmethyl)benzamide;
2-[3-{[4-Chloro-3-{[(4-chloro-3-(morpholin-4-ylmethyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-({4-chloro-3-[(cyclopropylamino) methyl]phenylethynyl)phenyl]-1-{3-[(3S)-3-methylmorpholin-4-yl]propyl}-1,4,6,7-tetrahydro-5H-pyrazolo[4,3-c]pyridin-5-yl)-2-oxoacetamide;
3-[4-Chloro-3-({4-chloro-3-[(2-morpholin-4-ylethyl)carbamoyl]phenyl}ethynyl)phenyl]-1-(3-piperidin-1-ylpropyl)-1,4,6,7-tetrahydro-5H-pyrazolo[4,3-c]pyridine-5-carboxamide;
5-[(5-{5-[Amino(oxo)acetyl]-1-(3-piperidin-1-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
2-{3-[4-Chloro-3-({4-chloro-3-[(ethylamino) methyl]phenylethynyl)phenyl]-1-(3-piperidin-1-ylpropyl)-1,4,6,7-tetrahydro-5H-pyrazolo[4,3-c]pyridin-5-yl}-2-oxoacetamide;
2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]ethanamine;
2-{3-[4-Chloro-3-({4-chloro-3-[(cyclopentylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
3-[4-Chloro-3-({4-chloro-3-[(cyclopropylamino)methyl]phenyl}ethynyl)phenyl]-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopentanamine;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(azetidin-3-ylmethyl)-2-chlorobenzamide;
3-[4-Chloro-3-(4-chloro-3-[(cyclopentylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
2-{3-[4-Chloro-3-(4-chloro-3-[(cyclopropylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
2-[3-{[4-Chloro-3-{[(4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridin-5-yl]-2-oxoacetamide;
3-(4-Chloro-3-{[4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopropanamine;
3-(4-Chloro-3-{[4-chloro-3-(morpholin-4-ylmethyl)phenyl]ethynyl]phenyl)-5-(methylsulfonyl)-1-(3-piperidin-1-ylpropyl)-4,5,6,7-tetrahydro-1H-pyrazolo[4,3-c]pyridine;
3-[4-Chloro-3-({4-chloro-3-[(cyclopropylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(morpholin-4-ylmethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-({4-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
3-{4-Chloro-3-[(4-{[(2S)-pyrrolidin-2-ylmethyl]carbamoylphenyl)ethynyl]phenyl}-1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-[(2S)-pyrrolidin-2-ylmethyl]benzamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[(2S)-pyrrolidin-2-ylmethyl]benzamide;
3-{4-Chloro-3-[(4-{[3-(methylamino)propyl]carbamoyl}phenyl)ethynyl]phenyl}1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-[3-(methylamino)propyl]benzamide; and
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[3-(methylamino)propyl]benzamide;
3-[4-Chloro-3-({4-chloro-3-[(dimethylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
2-(3-{4-Chloro-3-({4-chloro-3-[(dimethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N,N-dimethylmethanamine;
2-[3-{[4-Chloro-3-{[(4-(hydroxymethyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-[(4-chloro-3-{[(1-methylethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-(3-{4-Chloro-3-[(4-{[(4-chlorobenzyl)amino]methyl{phenyl)ethynyl]phenyl}-1-{3-[(3R)-3-methylmorpholin-4-yl]propyl}-1,4,6,7-tetrahydro-5H-pyrazolo[4,3-c]pyridin-5-yl)-2-oxoacetamide;
2-(3-{4-Chloro-3-[(4-{[(4-chlorobenzyl)amino]methyl-phenyl)ethynyl]phenyl}-1-[3-(3-methylmorpholin-4-yl)propyl]-1,4,6,7-tetrahydro-5H-pyrazolo[4,3-c]pyridin-5-yl)-2-oxoacetamide;
and pharmaceutically acceptable salts thereof.

64. A method according to claim 60, wherein said chemical entity is selected from the group consisting of:
4-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}butan-1-ol;
5-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}pentan-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}propan-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}propan-1-amine;
1-[1-(3-{3-[3-(3-Aminopropyl)-4-chlorophenyl]-5-(methylsulfonyl)-4,5,6,7-tetrahydro-1H-pyrazolo[4,3-c]pyridin-1-yl}propyl)piperidin-4-yl]pyrrolidin-2-one;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynylamine; and
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propylamine;
and pharmaceutically acceptable salts thereof.

65. A method according to claim 60, wherein the disease, disorder, or medical condition is an autoimmune disease, an allergic condition, inflammation, a bowel disorder, tissue transplant rejection, pain, or cancer.

66. A method according to claim 60, wherein the disease, disorder, or medical condition is selected from the group consisting of: lupus, asthma, allergic reaction, atopic allergy, hay fever, atopic dermatitis, food allergy, rhinitis, skin immune system disorders, psoriasis, uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease, chronic obstructive pulmonary disorder, tissue transplant rejection, pain, chronic pain, and cancer.

67. A method according to claim 60, wherein the disease, disorder, or medical condition is selected from the group consisting of: psoriasis, pain, multiple sclerosis, atherosclerosis, and rheumatoid arthritis.

68. A method for modulating cathepsin S activity, comprising exposing cathepsin S to an effective amount of at least one chemical entity selected from compounds of Formula (I), pharmaceutically acceptable salts of compounds of Formula (I), pharmaceutically acceptable prodrugs of compounds of Formula (I), and pharmaceutically active metabolites of compounds of Formula (I): 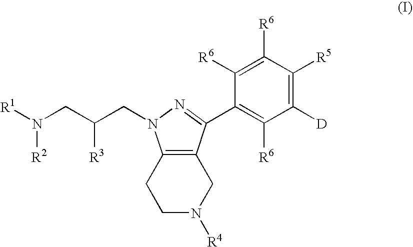
wherein:
R¹ and R² taken together with the nitrogen to which they are attached form a saturated monocyclic heterocycloalkyl group, each group optionally containing one additional heteroatom ring member that is O, S, or NR^{a}, and each group unsubstituted or substituted with one, two, or three R^{b} substituents;
where R^{a} is H, C₁₋₄alkyl, —COC₁₋₄alkyl, —CO(phenyl), or —CO₂C₁₋₄alkyl, or a monocyclic cycloalkyl ring, phenyl ring, or monocyclic heteroaryl ring, each ring unsubstituted or substituted with OH, C₁₋₄alkyl, CF₃, halo, —OC₁₋₄alkyl, cyano, or —COC₁₋₄alkyl; and
each R substituent is independently:
i) OH, C₁₋₄alkyl, —C₁₋₄alkyl-OH, CF₃, —NR^{c}R^{d}, halo, —OC₁₋₄alkyl, —COC₁₋₄alkyl, —CO₂C₁₋₄alkyl, —CO₂H, or —CONR^{e}R^{f};
ii) a monocyclic heterocycloalkyl group unsubstituted or substituted with C₁₋₄alkyl, —COC₁₋₄alkyl, —CO₂C₁₋₄alkyl, OH, —OC₁₋₄alkyl, —NR^{c}R^{d}, or halo;
iii) a monocyclic heterocycloalkyl group fused with a phenyl or pyridyl group, the resulting fused bicyclic group being unsubstituted or substituted with C₁₋₄alkyl, OH, —OC₁₋₄alkyl, —NR^{c}R^{d}, or halo; or
iv) a phenyl group or monocyclic heteroaryl group, each group unsubstituted or substituted with C₁₋₄alkyl, OH, —OC₁₋₄alkyl, —NR^{c}R^{d}, or halo; or
v) two R^{b} substituents on the same carbon taken together with the carbon to which they are attached form a saturated monocyclic heterocycloalkyl group, unsubstituted or substituted with C₁₋₄alkyl, OH, —OC₁₋₄alkyl, —NR^{c}R^{d}, or halo;
vi) two R^{b} substituents form a methylene or ethylene bridge; or
vii) two R^{b} substituents on adjacent carbons taken together with the carbons to which they are attached form a saturated monocyclic cycloalkyl group or saturated monocyclic heterocycloalkyl group, each group unsubstituted or substituted with C₁₋₄alkyl, OH, —OC₁₋₄alkyl, —NR^{c}R^{d}, or halo;
where R^{c} is H or C₁₋₄alkyl;
R^{d} is H, C₁₋₄alkyl, —COC₁₋₄alkyl, —COC₁₋₄alkyl-OH, —CO₂C₁₋₄alkyl, —CONR^{x}R^{y}, or —SO₂C₁₋₄alkyl;
where R^{x} and R^{y} are each independently H or C₁₋₄alkyl; and
R^{e} and R^{f} are each independently H or C₁₋₄alkyl;
R³ is H, OH, C₁₋₄alkyl, or —OC₁₋₄alkyl;
R⁴ is H; C₁₋₄alkyl; —COC₁₋₄alkyl unsubstituted or substituted with OH, F, —OCOC₁₋₄alkyl, or —NR^{t}R^{u}; —COCF₃; —CO-(monocyclic heteroaryl); —CO—(C-linked monocyclic heterocycloalkyl); —CO-(phenyl); —SO₂C₁₋₄alkyl; —SO₂CF₃; —SO₂NR^{t}R^{u}; —CONR^{t}R^{u}; —COCO₂C₁₋₄alkyl; or —COCONR^{t}R^{u};
where R^{t} and R^{u} are each independently H, C₁₋₄alkyl, or —COC₁₋₄alkyl; or R^{t} and R^{u} taken together with the nitrogen to which they are attached form a monocyclic heterocycloalkyl ring;
R⁵ is halo or CF₃;
each R⁶ is independently H or F;
D is —C≡C—R⁷, —CH═CH—R³, —(CH₂)₂₋₃—R³, or —(CH₂)₃₋₅—R⁹;
where R⁷ is:
I) a C₁₋₄alkyl group unsubstituted or substituted with OH, —OC₁₋₄alkyl, —NR^{g}R^{h}, phenyl, or phenoxy, each phenyl or phenoxy being unsubstituted or substituted with C₁₋₄alkyl, OH, —OC₁₋₄alkyl, halo, or CF₃;
where R^{g} and R^{h} are each independently H, C₁₋₄alkyl, —COC₁₋₄alkyl, —COphenyl, —CO₂C₁₋₄alkyl, —SO₂C₁₋₄alkyl, or —SO₂-phenyl; or R^{g} and R^{h} taken together with the nitrogen to which they are attached form a monocyclic heterocycloalkyl group; or
II) a monocyclic cycloalkyl group, phenyl group, or monocyclic heteroaryl group, each group unsubstituted or substituted with one or two R^{k} substituents;
where R⁸ is a phenyl group or monocyclic heteroaryl group, each group unsubstituted or substituted with one or two R^{k} substituents;
where R⁹ is OH or —NRⁿR^{o};
where Rⁿ is H or C₁₋₄alkyl; and
R^{o} is H, C₁₋₄alkyl, monocyclic cycloalkyl, —COC₁₋₄alkyl, —COphenyl, —CO₂C₁₋₄alkyl, —SO₂C₁₋₄alkyl, —SO₂-phenyl, —SO₂-benzyl, or —SO₂NR^{p}R^{g}, each phenyl or benzyl group being unsubstituted or substituted with one or two R^{k} substituents;
where R^{p} and R^{g} are each independently H or C₁₋₄alkyl;
or Rⁿ and R^{o} taken together with the nitrogen to which they are attached form a monocyclic saturated heterocycloalkyl ring unsubstituted or substituted with C₁₋₄alkyl, OH, —OC₁₋₄alkyl, halo, or CF₃;
where each R^{k} substituent in D is independently:
a) a C₁₋₄alkyl group unsubstituted or substituted with OH, —OC₁₋₄alkyl, —OC₁₋₄alkyl-OH, halo, —CO₂C₁₋₄alkyl, CO₂H, CN, —NR^{r}R^{s}, —N(R^{r})COphenyl, —N(R^{r})SO₂C₁₋₄alkyl, —N(R^{r}) SO₂-phenyl, —SO₂C₁₋₄alkyl, phenyl, or phenoxy;
b) a substituent of formula 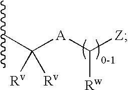
where each R^{v} is independently H or C₁₋₄alkyl, or both R^{v} substituents together form a carbonyl;
R^{w} is H, C₁₋₄alkyl, —CH₂OH, or —CO₂C₁₋₄alkyl;
A is O or NR^{aa};
where R^{aa} is H or C₁₋₄alkyl; and
Z is a phenyl group, benzyl group, cycloalkyl group, heterocycloalkyl group, heteroaryl group, or —CH₂— (heteroaryl) group, each group unsubstituted or substituted with one or two substituents independently selected from the group consisting of C₁₋₄alkyl, CF₃, halo, OH, —OC₁₋₄alkyl, —OCF₃, —OCHF₂, NR^{dd}R^{ee}, —CO₂C₁₋₄alkyl, —SC₁₋₄alkyl, and —SO₂C₁₋₄alkyl;
where R^{dd} and R^{ee} are each independently H or C₁₋₄alkyl;
c) two adjacent R^{k} substituents taken together with the carbons to which they are attached form a fused phenyl ring, monocyclic heteroaryl ring, monocyclic heterocycloalkyl ring, or monocyclic cycloalkyl ring, each fused ring unsubstituted or substituted with: C₁₋₄alkyl, —C₁₋₄alkyl-CF₃, —C₁₋₄alkyl-OH, —C₁₋₄alkyl-CO₂C₁₋₄alkyl, CF₃, C₂₋₄alkenyl, halo, OH, —OC₁₋₄alkyl, —COC₁₋₄alkyl, —COCF₃, —CO₂C₁₋₄alkyl, —CO₂H, —CONR^{ff}R^{gg}, or —SO₂C₁₋₄alkyl; or with a cycloalkyl group, —CH₂-(cycloalkyl) group, or benzyl group, each group unsubstituted or substituted with C₁₋₄alkyl, OH, —OC₁₋₄alkyl, halo, or CF₃;
where R^{ff} and R^{gg} are each independently H or C₁₋₄alkyl, or R^{ff} and R^{gg} taken together with the nitrogen to which they are attached form a monocyclic heterocycloalkyl ring, unsubstituted or substituted with C₁₋₄alkyl or OH; or
d) OH; —OC₁₋₄alkyl; halo; CF₃; —CHO; —CO₂C₁₋₄alkyl; CO₂H; CN; —NO₂; —CONR^{r}R^{s}, —NR^{r}R^{s}; —N(R^{r})_phenyl; —N(R^{r})-benzyl; —N(R^{r})-phenethyl; —N(R^{r})COC₁₋₄-alkyl; —N (R^{r}) CO-phenyl; —N(R^{r})SO₂C₁₋₄alkyl; —N (R^{r})SO₂-phenyl; —SO₂C₁₋₄alkyl; phenoxy; or a heteroaryl group; where each phenyl, benzyl, phenethyl, phenoxy, or heteroaryl group is unsubstituted or substituted with C₁₋₄alkyl, OH, —OC₁₋₄alkyl, halo, or CF₃;
where R^{r} is H, C₁₋₄alkyl, C₂₋₄alkyl-OH; and
R^{s} is H, C₁₋₄alkyl, —C₁₋₄alkyl-CF₃, —C₁₋₄alkyl-CN, —C₂₋₄alkyl-OH, —C₂₋₄alkyl-NR^{bb}R^{cc}, —C₁₋₄alkylCO₂C₁₋₄alkyl, —C₁₋₄alkylCO₂H, C₃₋₄alkenyl, —COC₁₋₄alkyl, or —CO₂C₁₋₄alkyl;
where R^{b} is H or C₁₋₄alkyl; and
R^{cc} is H, C₁₋₄alkyl, —COC₁₋₄alkyl, or —CO₂C₁₋₄alkyl;
or R^{b} and R^{cc} taken together with the nitrogen to which they are attached from a monocyclic heterocycloalkyl ring;
or R^{r} and R^{s} taken together with the nitrogen to which they are attached form a heterocycloalkyl group unsubstituted or substituted with C₁₋₄alkyl, OH, —OC₁₋₄alkyl, halo, CF₃, or a monocyclic heterocycloalkyl ring unsubstituted or substituted with OH.

69. A method according to claim 68, wherein the cathepsin S is in a subject with a disease, disorder, or medical condition mediated by cathepsin S activity.

70. A method according to claim 69, wherein the disease, disorder, or medical condition is an autoimmune disease, an allergic condition, inflammation, a bowel disorder, tissue transplant rejection, pain, or cancer.

71. A method according to claim 69, wherein the disease, disorder, or medical condition is selected from the group consisting of: lupus, asthma, allergic reaction, atopic allergy, hay fever, atopic dermatitis, food allergy, rhinitis, skin immune system disorders, psoriasis, uveitis, inflammation, upper airway inflammation, Sjögren's syndrome, arthritis, rheumatoid arthritis, osteoarthritis, type I diabetes, atherosclerosis, multiple sclerosis, coeliac disease, inflammatory bowel disease, chronic obstructive pulmonary disorder, tissue transplant rejection, pain, chronic pain, and cancer.

72. A method as in claim 69, wherein the disease, disorder, or medical condition is psoriasis, pain, multiple sclerosis, atherosclerosis, or rheumatoid arthritis.

73. A method according to claim 68, wherein said chemical entity is selected from the group consisting of:
3-[4-Chloro-3-(1H-indol-5-ylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[2-(1H-indol-5-yl)-ethyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetonitrile;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-methanol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
4-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-butyric acid;
3-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-propionic acid;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid methyl ester;
3-(4-Chloro-3-thiophen-2-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3,4-dichloro-phenylethynyl)-phenyl]-5-methanesulfonyl-1-(3-morpholin-4-yl-propyl)-4,5,6,7-tetrahydro-1H-pyrazolo[4,3-c]pyridine;
3-{4-Chloro-3-[4-(4-iodo-phenoxy)-phenylethynyl]-phenyl}-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetic acid;
3-[4-Chloro-3-(4-phenoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[3-(4-Bromo-phenylethynyl)-4-chloro-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzoic acid;
3-(4-Chloro-3-pyridin-4-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-3-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-pyridin-2-ylethynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-thiophen-3-ylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(2-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenol;
3-[4-Chloro-3-(4-chloro-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-p-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(4-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-fluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-methoxy-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2,4-difluoro-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(2-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
3-(4-Chloro-3-o-tolylethynyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-trifluoromethyl-phenylethynyl)-phenyl]-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzaldehyde;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenylamine;
3-(4-Chloro-3-phenylethynyl-phenyl)-5-methanesulfonyl-1-(3-morpholin-4-yl-propyl)-4,5,6,7-tetrahydro-1H-pyrazolo[4,3-c]pyridine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-carbamic acid tert-butyl ester;
3-[4-Chloro-3-(3-phenyl-prop-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-phenyl-but-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(5-phenyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-yn-1-ol;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-but-3-yn-1-ol;
5-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-pent-4-yn-1-ol;
3-(4-Chloro-3-hex-1-ynyl-phenyl)-5-methanesulfonyl-1-(3-morpholin-4-yl-propyl)-4,5,6,7-tetrahydro-1H-pyrazolo[4,3-c]pyridine;
3-(4-Chloro-3-cyclohexylethynyl-phenyl)-5-methanesulfonyl-1-(3-morpholin-4-yl-propyl)-4,5,6,7-tetrahydro-1H-pyrazolo[4,3-c]pyridine;
(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-diethyl-amine;
3-{4-Chloro-3-[3-(1,1-dioxo-1 A⁶-thiomorpholin-4-yl)-prop-1-ynyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(4-methyl-pent-1-ynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(3-phenoxy-prop-1-ynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-prop-2-ynyl)-benzenesulfonamide;
3-{4-Chloro-3-[2-(4-phenoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenol;
3-{4-Chloro-3-[2-(4-chloro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-{4-Chloro-3-[2-(4-methoxy-phenyl)-ethyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-[2-(2,4-difluoro-phenyl)-ethyl]-phenyl}-5-methanesulfonyl-1-(3-morpholin-4-yl-propyl)-4,5,6,7-tetrahydro-1H-pyrazolo[4,3-c]pyridine;
3-[4-Chloro-3-(2-o-tolyl-ethyl)-phenyl]-5-methanesulfonyl-1-(3-morpholin-4-yl-propyl)-4,5,6,7-tetrahydro-1H-pyrazolo[4,3-c]pyridine;
[4-(2-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-ethyl)-phenyl]-methanol;
3-(4-Chloro-3-phenethyl-phenyl)-5-methanesulfonyl-1-(3-morpholin-4-yl-propyl)-4,5,6,7-tetrahydro-1H-pyrazolo[4,3-c]pyridine;
3-[4-Chloro-3-(3-phenyl-propyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-phenyl)-acetamide;
3-(4-Chloro-3-Z-styryl-phenyl)-5-methanesulfonyl-1-(3-morpholin-4-yl-propyl)-4,5,6,7-tetrahydro-1H-pyrazolo[4,3-c]pyridine;
3-(4-Chloro-3-E-styryl-phenyl)-5-methanesulfonyl-1-(3-morpholin-4-yl-propyl)-4,5,6,7-tetrahydro-1H-pyrazolo[4,3-c]pyridine;
4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzylamine;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methanesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzenesulfonamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-acetamide;
N-(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-benzamide;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
(4-{2-Chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-(4-methyl-benzyl)-amine;
(4-Chloro-benzyl)-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-amine;
Benzyl-(4-{2-chloro-5-[5-methanesulfonyl-1-(3-morpholin-4-yl-propyl)-4,5,6,7-tetrahydro-1H-pyrazolo[4,3-c]pyridin-3-yl]-phenylethynyl}-benzyl)-methyl-amine;
3-[4-Chloro-3-(4-pyrrolidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
3-[4-Chloro-3-(4-piperidin-1-ylmethyl-phenylethynyl)-phenyl]-5-methanesulfonyl-1-(3-morpholin-4-yl-propyl)-4,5,6,7-tetrahydro-1H-pyrazolo[4,3-c]pyridine;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-methanesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-C-phenyl-methanesulfonamide;
3,4-Dichloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
4-Chloro-N-(3-{2-chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methyl-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-4-methoxy-benzenesulfonamide;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-N,N-dimethyl-sulfamoylurea;
N-(3-{2-Chloro-5-[5-methanesulfonyl-1-(3-morpholin-4-yl-propyl)-4,5,6,7-tetrahydro-1H-pyrazolo[4,3-c]pyridin-3-yl]-phenyl}-propyl)-acetamide;
2-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propylsulfamoyl}-benzoic acid;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-nitro-benzenesulfonamide;
3-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-4-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methoxy-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methyl-benzenesulfonamide;
2-Chloro-N-{3-[2-chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-nitro-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-cyano-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-3-methanesulfonyl-benzenesulfonamide;
N-{3-[2-Chloro-5-(5-methanesulfonyl-1-{3-[4-(2-oxo-pyrrolidin-1-yl)-piperidin-1-yl]-propyl}-4,5,6,7-tetrahydro-1H-pyrazolo[4,3-c]pyridin-3-yl)-phenyl]-propyl}-2-methanesulfonyl-benzenesulfonamide;
1-[1-(3-{3-[4-Chloro-3-(3-pyrrolidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-piperidin-1-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(3-methyl-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[3-(4,4-difluoro-piperidin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(3-morpholin-4-yl-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[3-(4-methyl-piperazin-1-yl)-propyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[3-(3-Azepan-1-yl-propyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(3-cyclopentylamino-propyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-pyrrolidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-piperidin-1-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-{1-[3-(3-{4-Chloro-3-[4-(3-methyl-piperidin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-{1-[3-(3-{4-Chloro-3-[4-(4-methyl-piperazin-1-yl)-butyl]-phenyl}-5-methanesulfonyl-4,5,6,7-tetrahydro-pyrazolo[4,3-c]pyridin-1-yl)-propyl]-piperidin-4-yl}-pyrrolidin-2-one;
1-[1-(3-{3-[4-Chloro-3-(4-morpholin-4-yl-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Azepan-1-yl-butyl)-4-chloro-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[4-Chloro-3-(4-cyclopentylamino-butyl)-phenyl]-5-methanesulfonyl-4,5,6,7-tetrahydro-pyrazolo[4,3-c]pyridin-1-yl}-propyl)-piperidin-4-yl]-pyrrolidin-2-one;
1-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-pyrrolidin-2-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
8-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-2,8-diaza-spiro[4.5]decan-1-one;
1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidine-4-carboxylic acid amide;
3-[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-1,3-dihydro-imidazo[4,5-b]pyridin-2-one;
[1-(3-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
1-{3-[3-(4-Chloro-phenylethynyl)-4-trifluoromethyl-phenyl]-5-methanesulfonyl-4,5,6,7-tetrahydro-pyrazolo[4,3-c]pyridin-1-yl}-3-morpholin-4-yl-propan-2-ol;
2-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-cyclopentanone;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-morpholin-4-yl-propan-2-ol;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperidin-1-yl-propan-2-ol;
3-[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-5-dimethylamino-1-methyl-1,3-dihydro-imidazo[4,5-b]pyridin-2-one;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid methyl ester;
1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidine-4-carboxylic acid amide;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-pyrrolidin-1-yl-propan-2-ol;
[1-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperidin-4-yl]-carbamic acid tert-butyl ester;
4-(3-{3-[4-Chloro-3-(4-chloro-phenylethynyl)-phenyl]-5-methanesulfonyl-4,5,6,7-tetrahydro-pyrazolo[4,3-c]pyridin-1-yl}-2-hydroxy-propyl)-piperazine-1-carboxylic acid tert-butyl ester;
1-{3-[4-Chloro-3-(4-chloro-phenylethynyl)-phenyl]-5-methanesulfonyl-4,5,6,7-tetrahydro-pyrazolo[4,3-c]pyridin-1-yl}-3-piperazin-1-yl-propan-2-ol;
1-(4-Amino-piperidin-1-yl)-3-{3-[4-chloro-3-(4-chloro-phenylethynyl)-phenyl]-5-methanesulfonyl-4,5,6,7-tetrahydro-pyrazolo[4,3-c]pyridin-1-yl}-propan-2-ol;
1-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-3-piperidin-1-yl-propan-2-ol;
1-(3-{3-[4-Chloro-3-(1,2,3,4-tetrahydro-isoquinolin-7-ylethynyl)-phenyl]-5-methanesulfonyl-4,5,6,7-tetrahydro-pyrazolo[4,3-c]pyridin-1-yl}-propyl)-piperidine-4-carboxylic acid amide;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-oxo-acetic acid methyl ester;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-pyridin-2-yl-methanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-furan-2-yl-methanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2,2,2-trifluoro-ethanone;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-fluoro-ethanone;
[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-(tetrahydro-furan-2-yl)-methanone;
Acetic acid 2-[3-[4-chloro-3-(4-chloro-phenylethynyl)-phenyl]-1-(3-morpholin-4-yl-propyl)-1,4,6,7-tetrahydro-pyrazolo[4,3-c]pyridin-5-yl]-2-oxo-ethyl ester;
1-[3-[4-Chloro-3-(4-chloro-phenylethynyl)-phenyl]-1-(3-morpholin-4-yl-propyl)-1,4,6,7-tetrahydro-pyrazolo[4,3-c]pyridin-5-yl]-2-hydroxy-ethanone;
3-[4-Chloro-3-(4-chloro-phenylethynyl)-phenyl]-1-(3-morpholin-4-yl-propyl)-1,4,6,7-tetrahydro-pyrazolo[4,3-c]pyridine-5-carboxylic acid amide;
3-{4-Chloro-3-[2-(4-chloro-phenyl)-ethyl]-phenyl}-1-(3-morpholin-4-yl-propyl)-1,4,6,7-tetrahydro-pyrazolo[4,3-c]pyridine-5-carboxylic acid amide;
1-{1-[3-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-2-hydroxy-propyl]-piperidin-4-yl}-pyrrolidin-2-one;
1-(3-{3-[2-(4-Chloro-phenyl)-ethyl]-4-trifluoromethyl-phenyl}-5-methanesulfonyl-4,5,6,7-tetrahydro-pyrazolo[4,3-c]pyridin-1-yl)-3-morpholin-4-yl-propan-2-ol;
8-[3-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-2-hydroxy-propyl]-2,8-diaza-spiro[4.5]decan-1-one; and
1-(3-{4-Chloro-3-[2-(4-chloro-phenyl)-ethyl]-phenyl}-5-methanesulfonyl-4,5,6,7-tetrahydro-pyrazolo[4,3-c]pyridin-1-yl)-3-morpholin-4-yl-propan-2-ol;
and pharmaceutically acceptable salts thereof.

74. A method according to claim 68, wherein said chemical entity is selected from the group consisting of:
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoacetamide;
3-(4-Chloro-3-[4-({[(4-chlorophenyl)methyl]aminomethyl)phenyl]ethynyl]phenyl)-1-(3-morpholin-4-ylpropyl)-1,4,6,7-tetrahydro-5H-pyrazolo[4,3-c]pyridine-5-carboxamide;
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-N,N-dimethyl-2-oxoacetamide;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-sulfonamide;
2-[3-{[4-Chloro-3-{[(4-({[(4-chlorophenyl)methyl]amino(methyl)phenyl]ethynyl}-phenyl)-1-(3-morpholin-4-ylpropyl)-1,4,6,7-tetrahydro-5H-pyrazolo[4,3-c]pyridin-5-yl]-2-oxoethanamine;
3-(4-Chloro-3-{[4-({[(4-chlorophenyl)methyl]amino]methyl)phenyl]ethynyl]phenyl)-N-methyl-1-(3-morpholin-4-ylpropyl)-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-pyridin-2-ylpiperazin-1-yl)propyl]-1,4,6,7-tetrahydro-5H-pyrazolo[4,3-c]pyridine-5-carboxamide;
2-(3-{4-Chloro-3-[(4-[(4-chlorobenzyl)amino]methylphenyl)ethynyl]phenyl}-1-{3-[(3S,5S)-3,5-dimethylmorpholin-4-yl]propyl}-1,4,6,7-tetrahydro-5H-pyrazolo[4,3-c]pyridin-5-yl)-2-oxoacetamide;
2-[3-{[4-Chloro-3-{[(4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-{3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridin-5-yl}-2-oxoacetamide;
3-(4-Chloro-3-{[4-({[(4-chlorophenyl)methyl]amino(methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(pyridin-3-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-{[(pyridin-3-ylmethyl)amino]carbonyl-phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-{[(piperidin-4-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[4-(2-oxopyrrolidin-1-yl)piperidin-1-yl]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
1-[4-({2-Chloro-5-[1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-({4-chloro-3-[(ethylamino)methyl]phenyl}ethynyl)phenyl]-1-(3-piperidin-1-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(2-morpholin-4-ylethyl)benzamide;
4-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(2-morpholin-4-ylethyl)benzamide;
3-{4-Chloro-3-[(4-{[(4-chlorobenzyl)amino]methyl(phenyl)ethynyl]phenyl}-1-{3-[(3aR,6aS)-tetrahydro-1H-furo[3,4-c]pyrrol-5(3H)-yl]propyl}-1,4,6,7-tetrahydro-5H-pyrazolo[4,3-c]pyridine-5-carboxamide;
3-(4-Chloro-3-{[3-({[(4-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoethanol;
3-(4-Chloro-3-{[4-([(4-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(1-methylethyl)oxy]phenyl}methyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-({4-[({[4-(dimethylamino)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-{4-Chloro-3-[(4-{[({4-[(difluoromethyl)oxy]phenyl]methyl)amino]methyl}phenyl)ethynyl]phenyl}-1-(3-thiomorpholin-4-ylpropyl)-1,4,6,7-tetrahydro-5H-pyrazolo[4,3-c]pyridine-5-carboxamide;
1-{4-[(2-Chloro-5-{5-(methylsulfonyl)-1-[3-(4-pyridin-2-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperidin-1-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-[4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)benzaldehyde;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydro-2H-pyran-4-ylmethyl)methanamine;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(tetrahydro-2H-pyran-4-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
Methyl N-{[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycine;
2-(1,1-Dimethylethyl) 3-methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-methyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N,N-dimethyl-1,2,3,4-tetrahydroisoquinoline-3-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxamide;
Methyl (3S)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
Methyl (3R)-7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylic acid;
2-(1,1-Dimethylethyl) 3-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-2,3(1H)-dicarboxylate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-3-carboxylate;
[6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinolin-3-yl]methanol;
2-(1,1-Dimethylethyl) 1-methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinoline-1,2(1H)-dicarboxylate;
Methyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline-1-carboxylate;
(2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
Methyl (2R)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3,4-dichlorophenyl)methyl]methanamine;
(1S,2R)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl]phenyl)ethynyl]phenyl}methyl)amino]-2,3-dihydro-1H-inden-1-ol;
(1R,2S)-1-[({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl methyl)amino]-2,3-dihydro-1H-inden-2-ol;
(1R)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1S)—N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(1R)—N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1,2,3,4-tetrahydronaphthalen-1-amine;
(2S)-2-[({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)amino]-2-phenylethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
Methyl N-{[5-({5-[5-(aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl}ethynyl)-2-chlorophenyl]methyl}glycinate;
N-{[5-({5-[5-(Aminocarbonyl)-1-{3-[(3S)-3-methylmorpholin-4-yl]propyl}-4,5,6,7-tetrahydro-1H-pyrazolo[4,3-c]pyridin-3-yl]-2-chlorophenyl]ethynyl)-2-chlorophenyl]methyl]glycine;
3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridine-5-carboxylic acid amide;
3-{4-Chloro-3-[(4-chloro-3-{[(tetrahydrofuran-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
3-{4-Chloro-3-[(4-chloro-3-{[(phenylmethyl)amino]methyl}phenyl)ethynyl]phenyl-1-{3-[(3S)-3-methylmorpholin-4-yl]propyl}-1,4,6,7-tetrahydro-5H-pyrazolo[4,3-c]pyridine-5-carboxamide;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-methyl-1,2,3,4-tetrahydroisoquinoline;
N-[1-(3-{3-[4-Chloro-3-(1,2,3,4-tetrahydroisoquinolin-7-ylethynyl)phenyl]-5-(methylsulfonyl)-4,5,6,7-tetrahydro-1H-pyrazolo[4,3-c]pyridin-1-yl}propyl)piperidin-4-yl]acetamide;
7-(2-{2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethyl)-1,2,3,4-tetrahydroisoquinoline;
7-[2-(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethyl]-1,2,3,4-tetrahydroisoquinoline;
1,1-Dimethylethyl 1′-{(2S)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
1,1-Dimethylethyl 1′-[(2S)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(cyclohexylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-[(2S)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl-2-hydroxypropyl]-4,4′-bipiperidine-1-carboxylate;
1,1-Dimethylethyl 1′-{(2S)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
1,1-Dimethylethyl 1′-{(2S)-3-[5-(aminocarbonyl)-3-{4-chloro-3-[(4-chlorophenyl)ethynyl]phenyl}-4,5,6,7-tetrahydro-1H-pyrazolo[4,3-c]pyridin-1-yl]-2-hydroxypropyl}-4,4′-bipiperidine-1-carboxylate;
1-[(2S)-3-(4,4′-Bipiperidin-1-yl)-2-hydroxypropyl]-3-{4-chloro-3-[(4-chlorophenyl)ethynyl]phenyl}-1,4,6,7-tetrahydro-5H-pyrazolo[4,3-c]pyridine-5-carboxamide;
(2S)-1-(4,4′-Bipiperidin-1-yl)-3-{3-[4-chloro-3-(4-methylpent-1-yn-1-yl)phenyl]-5-(methylsulfonyl)-4,5,6,7-tetrahydro-1H-pyrazolo[4,3-c]pyridin-1-yl}propan-2-ol;
(2S)-1-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(2S)-1-(1′-Acetyl-4,4′-bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
1,1-Dimethylethyl 1′-{(2S)-3-[5-(aminocarbonyl)-3-{4-chloro-3-[2-(4-chlorophenyl)ethyl]phenyl}-4,5,6,7-tetrahydro-1H-pyrazolo[4,3-c]pyridin-1-yl]-2-hydroxypropyl}-4,4′-bipiperidine-1-carboxylate;
2-[3-(4-Chloro-3-{2-[4-({[(4-chlorophenyl)methyl]amino{methyl)phenyl]ethyl]phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
3-(4-Chloro-3-{2-[4-({[(4-chlorophenyl)methyl]amino]methyl)phenyl]ethyl]phenyl)-1-{3-[(3S)-3-methylmorpholin-4-yl]propyl}-1,4,6,7-tetrahydro-5H-pyrazolo[4,3-c]pyridine-5-carboxamide;
1,1-Dimethylethyl 1′-{(2S)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]-2-hydroxypropyl}-4,4′-bipiperidine-1-carboxylate;
(2S)-1-(4,4′-Bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-(1′-Acetyl-4,4′-bipiperidin-1-yl)-3-[3-{4-chloro-3-[2-(4-chlorophenyl)ethyl]phenyl}-5-(methylsulfonyl)-4,5,6,7-tetrahydro-1H-pyrazolo[4,3-c]pyridin-1-yl]propan-2-ol;
(2S)-1-[3-{4-Chloro-3-[2-(4-chlorophenyl)ethyl]phenyl}-5-(methylsulfonyl)-4,5,6,7-tetrahydro-1H-pyrazolo[4,3-c]pyridin-1-yl]-3-(1′-methyl-4,4′-bipiperidin-1-yl)propan-2-ol;
(S)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridin-5-yl]-2-oxo-acetamide;
(S)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-thiomorpholin-4-yl-propyl)-1,4,6,7-tetrahydro-pyrazolo[4,3-c]pyridine-5-carboxylic acid amide;
(S)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(R)-2-[3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridin-5-yl]-2-oxo-acetamide;
(2-{3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
2-{3-(4-Chloro-3-{4-[(4-chloro-benzylamino)-methyl]-phenylethynyl}-phenyl)-1-[(2R)-2-hydroxy-3-((3S)-3-methyl-morpholin-4-yl)-propyl]-1,4,6,7-tetrahydro-pyrazolo[4,3-c]pyridin-5-yl}-2-oxo-acetamide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
3-(4-Chloro-3-{4-[(4-chloro-benzylamino)-methyl]-phenylethynyl}-phenyl)-1-[(2S)-2-hydroxy-3-((3S)-3-methyl-morpholin-4-yl)-propyl]-1,4,6,7-tetrahydro-pyrazolo[4,3-c]pyridine-5-carboxylic acid amide;
(S)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(R)-3-(4-Chloro-3-{4-[(4-chloro-benzylamino)-methyl]-phenylethynyl}-phenyl)-1-(2-hydroxy-3-morpholin-4-yl-propyl)-1,4,6,7-tetrahydro-pyrazolo[4,3-c]pyridine-5-carboxylic acid amide;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-thiomorpholin-4-yl-propan-2-ol;
(S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(R)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-morpholin-4-yl-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
(2S)-1-[3-(4-Chloro-3-{4-[(4-chloro-benzylamino)-methyl]-phenylethynyl}-phenyl)-5-methanesulfonyl-4,5,6,7-tetrahydro-pyrazolo[4,3-c]pyridin-1-yl]-3-((3S)-3-methyl-morpholin-4-yl)-propan-2-ol;
2-{3-[4-Chloro-3-(4-chloro-3-{[((2S)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
2-{3-[4-Chloro-3-(4-chloro-3-{[((2R)-tetrahydro-furan-2-ylmethyl)-amino]-methyl}-phenylethynyl)-phenyl]-1-[3-((3S)-3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-cyclopentylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[4-Chloro-3-(4-chloro-3-propylaminomethyl-phenylethynyl)-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-2-{3-(4-Chloro-3-{4-chloro-3-[(3-hydroxy-propylamino)-methyl]-phenylethynyl}-phenyl)-1-[3-(3-methyl-morpholin-4-yl)-propyl]-1,4,6,7-tetrahydro-pyrazolo[4,3-c]pyridin-5-yl}-2-oxo-acetamide;
(S)-2-{3-[3-(3-{[Bis-(2-hydroxy-ethyl)-amino]-methyl}-4-chloro-phenylethynyl)-4-chloro-phenyl]-1-[3-(3-methyl-morpholin-4-yl)-propyl]-1,4,6,7-tetrahydro-pyrazolo[4,3-c]pyridin-5-yl}-2-oxo-acetamide;
(S)-5-(5-{5-Aminooxalyl-1-[3-(3-methyl-morpholin-4-yl)-propyl]-4,5,6,7-tetrahydro-1H-pyrazolo[4,3-c]pyridin-3-yl}-2-chloro-phenylethynyl)-2-chloro-N-(3-methylamino-propyl)-benzamide;
(S)-2-{3-{4-Chloro-3-[4-chloro-3-(3-hydroxy-propoxymethyl)-phenylethynyl]-phenyl}-1-[3-(3-methyl-morpholin-4-yl)-propyl]-1,4,6,7-tetrahydro-pyrazolo[4,3-c]pyridin-5-yl}-2-oxo-acetamide;
(S)-2-[3-[4-Chloro-3-[4-chloro-3-(pyrrolidin-3-yloxymethyl)-phenylethynyl]-phenyl]-1-[3-(3-methyl-morpholin-4-yl)-propyl]-1,4,6,7-tetrahydro-pyrazolo[4,3-c]pyridin-5-yl}-2-oxo-acetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-(1-methylethyl)-1,2,3,4-tetrahydroisoquinoline;
7-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]-2-cyclopropyl-1,2,3,4-tetrahydroisoquinoline;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridine-5-carboxamide;
2-(3-{4-Chloro-3-[(2-cyclopropyl-1,2,3,4-tetrahydroisoquinolin-7-yl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
2-(3-{4-Chloro-3-[(4-chloro-3-{[(phenylmethyl)amino]methyl-phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-5-(2-{2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine;
2-(3-{4-Chloro-3-[(4-chloro-3-{[(pyridin-2-ylmethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-(phenylmethyl)methanamine;
3-{4-Chloro-3-[(4-chloro-3-[(phenylmethyl)amino]methyl(phenyl)ethynyl]phenyl}-1-(3-thiomorpholin-4-ylpropyl)-1,4,6,7-tetrahydro-5H-pyrazolo[4,3-c]pyridine-5-carboxamide;
3-{4-Chloro-3-[(4-chloro-3-[(pyridin-2-ylmethyl)amino]methyl(phenyl)ethynyl]phenyl}-1-(3-thiomorpholin-4-ylpropyl)-1,4,6,7-tetrahydro-5H-pyrazolo[4,3-c]pyridine-5-carboxamide;
2-({[2-Chloro-5-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]methyl]amino)ethanol;
Methyl N-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)methyl]glycinate; and
2-(3-{4-Chloro-3-[(4-chloro-3-{[(2-hydroxyethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
and pharmaceutically acceptable salts thereof.

75. A method according to claim 68, wherein said chemical entity is selected from the group consisting of:
3-(2-{3-[5-(Methylsulfonyl)-1-(3-morpholin-4-ylpropyl)-4,5,6,7-tetrahydro-1H-pyrazolo[4,3-c]pyridin-3-yl]phenyl}ethyl)phenol;
3-{4-Chloro-3-[(4-chlorophenyl)ethynyl]phenyl}-1-(3-morpholin-4-ylpropyl)-4,5,6,7-tetrahydro-1H-pyrazolo[4,3-c]pyridine;
8-{3-[3-{4-Chloro-3-[(4-chlorophenyl)ethynyl]phenyl}-5-(methylsulfonyl)-4,5,6,7-tetrahydro-1H-pyrazolo[4,3-c]pyridin-1-yl]-2-hydroxypropyl}-2,8-diazaspiro[4.5]decan-1-one;
4-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}but-3-yn-1-ol;
3-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}prop-2-yn-1-amine;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl}prop-2-yn-1-yl)benzenesulfonamide;
N-(3-{5-[1-(2-Hydroxy-3-morpholin-4-ylpropyl)-5-(methylsulfonyl)-4,5,6,7-tetrahydro-1H-pyrazolo[4,3-c]pyridin-3-yl]-2-(trifluoromethyl)phenyl]propyl)benzenesulfonamide;
1-[1-(3-{3-[3-(3-Aminoprop-1-yn-1-yl)-4-chlorophenyl]-5-(methylsulfonyl)-4,5,6,7-tetrahydro-1H-pyrazolo[4,3-c]pyridin-1-yl}propyl)piperidin-4-yl]pyrrolidin-2-one;
Methyl 2-{[(3-{2-chloro-5-[5-(methylsulfonyl)-1-{3-[4-(2-oxopyrrolidin-1-yl)piperidin-1-yl]propyl}-4,5,6,7-tetrahydro-1H-pyrazolo[4,3-c]pyridin-3-yl]phenyl}propyl)amino]sulfonyl}benzoate;
1-[1-(3-{3-[4-Chloro-3-(3-hydroxyprop-1-yn-1-yl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-[1-(3-{3-[4-Chloro-3-(4-hydroxybutyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-{4-Chloro-3-[4-(dimethylamino)butyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[1-(3-{3-[4-Chloro-3-(3-hydroxypropyl)phenyl]-5-(methylsulfonyl)-4,5,6,7-tetrahydro-1H-pyrazolo[4,3-c]pyridin-1-yl}propyl)piperidin-4-yl]pyrrolidin-2-one;
1-(1-{3-[3-{4-Chloro-3-[3-(dimethylamino)propyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methylmethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl; —N-ethylethanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenylaethynyl)phenyl]methyl}-2-methylpropan-1-amine;
1-[4-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-[(4-chlorophenyl)methyl]methanamine;
3-(3-{[4-(1H-Benzimidazol-2-yl)phenyl]ethynyl]-4-chlorophenyl)-5-(methylsulfonyl)-1-(3-morpholin-4-ylpropyl)-4,5,6,7-tetrahydro-1H-pyrazolo[4,3-c]pyridine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(phenylmethyl)aniline; {[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]amino]acetonitrile;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclopropanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]cyclobutanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclopentanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}cyclohexanamine;
4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-N-(2-phenylethyl)aniline;
1-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1,1-Dimethylethyl (1-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}piperidin-4-yl)carbamate;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-ol;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-amine;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-morpholin-4-ylpropan-2-ol;
1-[4-({2-Chloro-5-[5-(methylsulfonyl)-1-(3-pyrrolidin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
Ethyl 1-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxylate;
1-{4-[(2-Chloro-5-{1-[3-(1,4-dioxa-8-azaspiro[4.5]dec-8-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{3-[3-(4-Chloro-3-{[4-({[(4-chlorophenyl)methyl]amino]methyl)phenylethynyl]phenyl)-5-(methylsulfonyl)-4,5,6,7-tetrahydro-1H-pyrazolo[4,3-c]pyridin-1-yl]propyl}piperidine-4-carboxylic acid;
(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanol;
1′-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-1,4′-bipiperidin-2-one;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methyloxy)phenyl]methyl}methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2,2,2-trifluoroethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(cyclopropylmethyl)methanamine;
(2S)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2-phenylethanol;
1-{4-[(2-Chloro-5-{5-(methylsulfonyl)-1-[3-(4-morpholin-4-ylpiperidin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4-methylpiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1-[4-({2-Chloro-5-[5-(methylsulfonyl)-1-{3-[4-(trifluoromethyl)piperidin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)acetamide;
Methyl N-{[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}glycinate;
1-(1-{3-[3-[4-Chloro-3-[(4-{[(2,2,2-trifluoroethyl)amino]methyl]phenyl)ethynyl]phenyl}-5-(methylsulfonyl)-4,5,6,7-tetrahydro-1H-pyrazolo[4,3-c]pyridin-1-yl]propyl}piperidin-4-yl)pyrrolidin-2-one;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}prop-2-en-1-amine;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidine-4-carboxamide;
Methyl (2S)-({[4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)(phenyl)ethanoate;
1-(1-{3-[3-(4-Chloro-3-{[4-({[(1R)-2-hydroxy-1-phenylethyl]aminomethyl)phenyl]ethynyl]phenyl)-5-(methylsulfonyl)-4,5,6,7-tetrahydro-1H-pyrazolo[4,3-c]pyridin-1-yl]propyl}piperidin-4-yl)pyrrolidin-2-one;
1-{4-[(5-{1-[3-(4-Acetylpiperazin-1-yl)propyl]-5-(methylsulfonyl)-4,5,6,7-tetrahydro-1H-pyrazolo[4,3-c]pyridin-3-yl}-2-chlorophenyl)ethynyl]phenyl}-N-[(4-chlorophenyl)methyl]methanamine;
1-{4-[(2-Chloro-5-{1-[3-(4-methylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4,4-dimethylpiperidin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)-2-hydroxyacetamide;
1-{4-[(2-Chloro-5-{1-[3-(4,4-difluoropiperidin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{1-[3-(4-fluoropiperidin-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperidin-4-yl)methanesulfonamide;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1-[3-(4-Chloro-3-{[4-([(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-piperidin-1-ylpropan-2-ol;
N-(1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidin-4-yl)acetamide;
1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-2-hydroxypropyl}piperidine-4-carboxamide;
3-(4-Chloro-3-{[2-(trifluoroacetyl)-2,3-dihydro-1H-isoindol-5-yl]ethynyl}phenyl)-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,2,3,4-tetrahydroisoquinoline;
1,1-Dimethylethyl 4-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazine-1-carboxylate;
1-[4-({2-Chloro-5-[5-(methylsulfonyl)-1-(3-piperazin-1-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
N-(1-{3-[3-(4-Chloro-3-{[4-({[(4-chlorophenyl)methyl]amino]methyl)phenyl]ethynyl]phenyl)-4,5,6,7-tetrahydro-1H-pyrazolo[4,3-c]pyridin-1-yl]propyl}piperidin-4-yl)acetamide;
1,1-Dimethylethyl 7-({2-chloro-5-[5-(methylsulfonyl)-1-(3-morpholin-4-ylpropyl)-4,5,6,7-tetrahydro-1H-pyrazolo[4,3-c]pyridin-3-yl]phenyl)ethynyl)-3,4-dihydroisoquinoline-2(1H)-carboxylate;
1,1-Dimethylethyl 7-({2-chloro-5-[1-(2-hydroxy-3-piperidin-1-ylpropyl)-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-[4-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1,1-Dimethylethyl 7-({5-[1-{3-[4-(aminocarbonyl)piperidin-1-yl]-2-hydroxypropyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
1,1-Dimethylethyl 7-({5-[1-{3-[4-(aminocarbonyl)piperidin-1-yl]propyl}-5-(methylsulfonyl)-4,5,6,7-tetrahydro-1H-pyrazolo[4,3-c]pyridin-3-yl]-2-chlorophenyl}ethynyl)-3,4-dihydroisoquinoline-2(1H)-carboxylate;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-3-benzazepine;
1,1-Dimethylethyl {[3-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
7-({2-Chloro-5-[1-{3-[4-(1,1-dimethylethyl)piperidin-1-yl]propyl}-5-(methylsulfonyl)-4,5,6,7-tetrahydro-1H-pyrazolo[4,3-c]pyridin-3-yl]phenyl}ethynyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[5-(methylsulfonyl)-1-{3-[4-(phenylcarbonyl)piperazin-1-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
7-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-1,2,3,4-tetrahydroisoquinoline;
(3S)-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
(3R)-1-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}pyrrolidin-3-ol;
1,1-Dimethylethyl {[2-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}carbamate;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methanamine;
1-[2-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[4-({2-Chloro-5-[1-{3-[(2R,6S)-2,6-dimethylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholin-2-yl)methanol;
1,1-Dimethylethyl 4-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}-1,4-diazepane-1-carboxylate;
1-{4-[(2-Chloro-5-{[1-[3-(1,4-diazepan-1-yl)propyl]-5-(methylsulfonyl)-4,5,6,7-tetrahydro-1H-pyrazolo[4,3-c]pyridin-3-yl]phenyl)ethynyl]phenyl}-N-[(4-chlorophenyl)methyl]methanamine;
1,1-Dimethylethyl 5-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1,3-dihydro-2H-isoindole-2-carboxylate;
3-[4-Chloro-3-(2,3-dihydro-1H-isoindol-5-ylethynyl)phenyl]-5-(methylsulfonyl)-1-(3-morpholin-4-ylpropyl)-4,5,6,7-tetrahydro-1H-pyrazolo[4,3-c]pyridine;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro-1,6-naphthyridine;
1-{4-[2-(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethyl]phenyl}-N-[(4-chlorophenyl)methyl]methanamine;
7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-cyclopropyl-1,2,3,4-tetrahydroisoquinoline;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-2-one;
1-{4-[(2-Chloro-5-{1-[3-(1,1-dioxidothiomorpholin-4-yl)propyl]-5-(methylsulfonyl)-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(1,4-oxazepan-4-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-(4-{[5-(5-Acetyl-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}phenyl)-N-[(4-chlorophenyl)methyl]methanamine;
3-(4-Chloro-3-{[4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridine-5-carboxamide;
2-[3-{[4-Chloro-3-{[(4-({[(4-chlorophenyl)methyl]amino{methyl)phenyl]ethynyl]phenyl)-1-{3-[(3S)-3-methylmorpholin-4-yl]propyl}-1,4,6,7-tetrahydro-5H-pyrazolo[4,3-c]pyridin-5-yl]-2-oxoethanol;
3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-5,6,7,8-tetrahydro[1,2,4]triazolo[4,3-a]pyrazine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-3-methylpiperazin-2-one;
2-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-2-ylmethyl)methanamine;
3-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
4-(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}piperazin-1-yl)phenol;
3-[3-(1H-Benzimidazol-5-ylethynyl)-4-chlorophenyl]-5-(methylsulfonyl)-1-(3-morpholin-4-ylpropyl)-4,5,6,7-tetrahydro-1H-pyrazolo[4,3-c]pyridine;
1-[4-({5-[5-Acetyl-1-(3-thiomorpholin-4-ylpropyl)-4,5,6,7-tetrahydro-1H-pyrazolo[4,3-c]pyridin-3-yl]-2-chlorophenyl]ethynyl)phenyl]-N-[(4-chlorophenyl)methyl]methanamine;
2-[3-{[4-Chloro-3-{[(4-({[(4-chlorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridin-5-yl]-2-oxoethanol;
2-[3-{[4-Chloro-3-{[(4-({[(4-chlorophenyl)methyl]amino]methyl)phenyl]ethynyl]phenyl)-1-(3-thiomorpholin-4-ylpropyl)-1,4,6,7-tetrahydro-5H-pyrazolo[4,3-c]pyridin-5-yl]-2-oxoacetamide;
7-[(2-Chloro-5-{1-[3-(4-cyclopropylpiperazin-1-yl)propyl]-5-(methylsulfonyl)-4,5,6,7-tetrahydro-1H-pyrazolo[4,3-c]pyridin-3-yl}phenyl)ethynyl]-1,2,3,4-tetrahydroisoquinoline;
1-{4-[(2-Chloro-5-{5-(methylsulfonyl)-1-[3-(4-pyridin-4-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
1-{4-[(2-Chloro-5-{5-(methylsulfonyl)-1-[3-(4-pyridin-3-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
Methyl 4-{3-[3-(4-chloro-3-{[4-({[(4-chlorophenyl)methyl]amino}methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylate;
(4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholin-3-yl)methanol;
1-[4-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(3-thienylmethyl)methanamine;
N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-(2-thienyl)ethanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[5-methyl-2-(trifluoromethyl)furan-3-yl]methyl}methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)pyridin-3-yl]-N-[(4-chlorophenyl)methyl]methanamine;
2-{3-(4-Chloro-3-{[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridin-5-yl}-2-oxoacetamide;
3-(4-Chloro-3-[4-({[(4-chlorophenyl)methyl]amino]methyl)phenyl]ethynyl]phenyl)-1-[3-(4-phenylpiperazin-1-yl)propyl]-1,4,6,7-tetrahydro-5H-pyrazolo[4,3-c]pyridine-5-carboxamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(3-methyl-2-thienyl)methyl]methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(furan-2-ylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(2-thienylmethyl)methanamine;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-(3-thienylmethyl)methanamine;
2-[3-{[4-Chloro-3-{[(4-({[(4-chlorophenyl)methyl]amino{methyl)phenyl]ethynyl]phenyl)-1-{3-[(1S,4S)-2-oxa-5-azabicyclo[2.2.1]hept-5-yl]propyl}-1,4,6,7-tetrahydro-5H-pyrazolo[4,3-c]pyridin-5-yl]-2-oxoacetamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-fluorophenyl]-N-[(4-chlorophenyl)methyl]methanamine;
4-{3-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}morpholine-3-carboxylic acid;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[3-({2-Chloro-5-[5-(methylsulfonyl)-1-{3-[(1S,4S)-2-oxa-5-azabicyclo[2.2.1]hept-5-yl]propyl}-4,5,6,7-tetrahydro-1H-pyrazolo[4,3-c]pyridin-3-yl]phenyl}ethynyl)phenyl]-N-[(4-chlorophenyl)methyl]methanamine;
3-[4-Chloro-3-(1H-imidazol-4-ylethynyl)phenyl]-5-(methylsulfonyl)-1-(3-morpholin-4-ylpropyl)-4,5,6,7-tetrahydro-1H-pyrazolo[4,3-c]pyridine;
2-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydroisoquinoline;
3-(4-Chloro-3-{[4-(1,3-dihydro-2H-isoindol-2-ylmethyl)phenyl]ethynyl}phenyl)-5-(methylsulfonyl)-1-(3-morpholin-4-ylpropyl)-4,5,6,7-tetrahydro-1H-pyrazolo[4,3-c]pyridine;
(1R)—N-{[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1,2,3,4-tetrahydronaphthalen-1-amine;
(1S,2R)-2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)-2,3-dihydro-1H-inden-1-ol;
1-{3-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]methanamine;
1-{3-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
(2S)-1-(4,4′-Bipiperidin-1-yl)-3-[3-{4-chloro-3-[(4-chlorophenyl)ethynyl]phenyl}-5-(methylsulfonyl)-4,5,6,7-tetrahydro-1H-pyrazolo[4,3-c]pyridin-1-yl]propan-2-ol;
2-[3-{[4-Chloro-3-{[(4-({[(4-chlorophenyl)methyl]amino(methyl)phenyl]ethynyl}-phenyl)-1-(3-piperidin-1-ylpropyl)-1,4,6,7-tetrahydro-5H-pyrazolo[4,3-c]pyridin-5-yl]-2-oxoacetamide;
1-(3-{5-[Amino(oxo)acetyl]-3-(4-chloro-3-{[4-({[(4-chlorophenyl)methyl]aminomethyl)phenyl]ethynyl]phenyl)-4,5,6,7-tetrahydro-1H-pyrazolo[4,3-c]pyridin-1-yl}propyl)piperidine-4-carboxamide;
2-{3-(4-Chloro-3-{[4-({[(4-chlorophenyl)methyl]amino-4-methyl)phenyl]ethynyl]phenyl)-1-[3-(1,4-dioxa-8-azaspiro[4.5]dec-8-yl)propyl]-1,4,6,7-tetrahydro-5H-pyrazolo[4,3-c]pyridin-5-yl}-2-oxoacetamide;
2-[3-(4-Chloro-3-{[4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridin-5-yl]-2-oxoacetamide;
3-(4-Chloro-3-{[4-({[(4-chlorophenyl)methyl]amino(methyl)phenyl]ethynyl}-phenyl)-1-(3-pyrrolidin-1-ylpropyl)-1,4,6,7-tetrahydro-5H-pyrazolo[4,3-c]pyridine-5-carboxamide;
3-(4-Chloro-3-[4-({[(4-chlorophenyl)methyl]amino(methyl)phenyl]ethynyl]phenyl)-1-{3-[4-(1-oxidopyridin-2-yl)piperazin-1-yl]propyl}-1,4,6,7-tetrahydro-5H-pyrazolo[4,3-c]pyridine-5-carboxamide;
3-(4-Chloro-3-[4-({[(4-chlorophenyl)methyl]amino]methyl)phenyl]ethynyl]phenyl)-1-{3-[4-(trifluoromethyl)piperidin-1-yl]propyl}-1,4,6,7-tetrahydro-5H-pyrazolo[4,3-c]pyridine-5-carboxamide;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(1,3-thiazol-2-ylmethyl)methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(1-methyl-1H-imidazol-5-yl)methyl]methanamine;
3-(4-Chloro-3-{[4-({[(4-chlorophenyl)methyl]amino(methyl)phenyl]ethynyl]phenyl)-1-(3-piperidin-1-ylpropyl)-1,4,6,7-tetrahydro-5H-pyrazolo[4,3-c]pyridine-5-carboxamide;
1-{3-[4-(Acetylamino)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-cyclobutylpiperazin-1-yl)propyl]-1,4,6,7-tetrahydro-5H-pyrazolo[4,3-c]pyridine-5-carboxamide;
1-{3-[4-(Aminocarbonyl)piperidin-1-yl]propyl}-3-(4-chloro-3-{[4-({[(4-chlorophenyl)methyl]amino}methyl)phenyl]ethynyl}phenyl)-1,4,6,7-tetrahydro-5H-pyrazolo[4,3-c]pyridine-5-carboxamide;
3-(4-Chloro-3-{[4-({[(4-chlorophenyl)methyl]amino-4-methyl)phenyl]ethynyl-phenyl)-1-[3-(1,4-dioxa-8-azaspiro[4.5]dec-8-yl)propyl]-1,4,6,7-tetrahydro-5H-pyrazolo[4,3-c]pyridine-5-carboxamide;
3-(4-Chloro-3-{[4-({[(4-chlorophenyl)methyl]amino]methyl)phenyl]ethynyl]phenyl)-1-[3-(4-cyclopropylpiperazin-1-yl)propyl]-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-{[(1,1-dimethylethyl)oxy]carbonyl}-1,2,3,4-tetrahydroisoquinoline-3-carboxylic acid;
3-[4-Chloro-3-({2-[(4-chlorophenyl)methyl]-1,2,3,4-tetrahydroisoquinolin-7-yl}ethynyl)phenyl]-1-(3-morpholin-4-ylpropyl)-1,4,6,7-tetrahydro-5H-pyrazolo[4,3-c]pyridine-5-carboxamide;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
1,1-Dimethylethyl 7-({2-chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-3,4-dihydroisoquinoline-2(1H)-carboxylate;
1-{4-[(2-Chloro-5-{5-(methylsulfonyl)-1-[3-(2-oxa-6-azaspiro[3.3]hept-6-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
2-({[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-({4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl}phenyl)ethynyl]phenyl}methyl)-2,3-dihydro-1H-inden-1-amine;
(2S)-1-(4,4′-Bipiperidin-1-yl)-3-[3-{4-chloro-3-[3-(diethylamino)prop-1-yn-1-yl]phenyl}-5-(methylsulfonyl)-4,5,6,7-tetrahydro-1H-pyrazolo[4,3-c]pyridin-1-yl]propan-2-ol;
(2S)-1-(4,4′-Bipiperidin-1-yl)-3-{3-[4-chloro-3-(cyclohexylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-2-ylethynyl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(3-phenylprop-1-yn-1-yl)phenyl]-5-(methylsulfonyl)-4,5,6,7-tetrahydro-1H-pyrazolo[4,3-c]pyridin-1-yl}propan-2-ol;
(2S)-1-(4,4′-Bipiperidin-1-yl)-3-{3-[4-chloro-3-(pyridin-3-ylethynyl)phenyl]-5-(methylsulfonyl)-4,5,6,7-tetrahydro-1H-pyrazolo[4,3-c]pyridin-1-yl}propan-2-ol;
3-(4-Chloro-3-{2-[4-({[(4-chlorophenyl)methyl]amino-methyl)phenyl]ethyl]phenyl)-1-(3-morpholin-4-ylpropyl)-1,4,6,7-tetrahydro-5H-pyrazolo[4,3-c]pyridine-5-carboxamide;
3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl}phenyl)ethynyl]phenyl}methyl)-1-phenylethanamine;
1-[(2S)-3-(4,4′-Bipiperidin-1-yl)-2-hydroxypropyl]-3-{4-chloro-3-[2-(4-chlorophenyl)ethyl]phenyl}-1,4,6,7-tetrahydro-5H-pyrazolo[4,3-c]pyridine-5-carboxamide;
N-({4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}methyl)-2,2,2-trifluoroethanamine;
7-{[2-Chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}-2-cyclopropyl-1,2,3,4-tetrahydroisoquinoline;
3-[4-Chloro-3-(1H-pyrrolo[2,3-b]pyridin-6-ylethynyl)phenyl]-5-(methylsulfonyl)-1-(3-morpholin-4-ylpropyl)-4,5,6,7-tetrahydro-1H-pyrazolo[4,3-c]pyridine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(pyrrolidin-1-ylcarbonyl)-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-[(4-methylpiperazin-1-yl)carbonyl]-1,2,3,4-tetrahydroisoquinoline;
(3S)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3-(piperidin-1-ylcarbonyl)-1,2,3,4-tetrahydroisoquinoline;
3-(4-Chloro-3-{[4-({[(3-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(2-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
8-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2,3,4,5-tetrahydro-1H-2-benzazepine;
1-{4-[(2-Chloro-5-{5-(methylsulfonyl)-1-[3-(4-phenylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]-N-methylmethanamine;
1,1-Dimethylethyl (1S,4S)-5-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1,1-Dimethylethyl (1R,4R)-5-{3-[3-(4-chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]propyl}-2,5-diazabicyclo[2.2.1]heptane-2-carboxylate;
1-[4-({2-Chloro-5-[1-{3-[(1S,4S)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[1-{3-[(1R,4R)-2,5-diazabicyclo[2.2.1]hept-2-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-[4-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
2-[3-{4-Chloro-3-[(4-{[(1R)-1,2,3,4-tetrahydronaphthalen-1-ylamino]methyl}phenyl)ethynyl]phenyl}-1-(3-morpholin-4-ylpropyl)-1,4,6,7-tetrahydro-5H-pyrazolo[4,3-c]pyridin-5-yl]-2-oxoacetamide;
1-[4-({2-Chloro-5-[5-(morpholin-4-ylcarbonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-{[4-({[(4-chlorophenyl)methyl]amino]methyl)phenyl]ethynyl]phenyl)-N,N-dimethyl-1-(3-morpholin-4-ylpropyl)-1,4,6,7-tetrahydro-5H-pyrazolo[4,3-c]pyridine-5-carboxamide;
N-{2-[3-(4-Chloro-3-{[4-({[(4-chlorophenyl)methyl]amino-methyl)phenyl]ethynyl]phenyl)-1-(3-morpholin-4-ylpropyl)-1,4,6,7-tetrahydro-5H-pyrazolo[4,3-c]pyridin-5-yl]-2-oxoethylacetamide;
1-[4-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
7-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl-4-ethynyl)-2-(2,2,2-trifluoroethyl)-1,2,3,4-tetrahydroisoquinoline;
1-{4-[(2-Chloro-5-{5-(methylsulfonyl)-1-[3-(4-pyridin-2-ylpiperazin-1-yl)propyl]-4,5,6,7-tetrahydro-1H-pyrazolo[4,3-c]pyridin-3-yl]phenyl)ethynyl]phenyl}-N-[(4-chlorophenyl)methyl]-N-methylmethanamine;
1-[4-({2-Chloro-5-[5-(methylsulfonyl)-1-(3-thiomorpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]-N-methylmethanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-2-phenylethanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(cyclopropylmethyl)-1,2,3,4-tetrahydroisoquinoline;
2-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl]amino)ethanol;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}-1-phenylethanamine;
3-(4-Chloro-3-[4-([(3-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-({[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl]ethynyl)phenyl]methyl]amino)propan-1-ol;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(tetrahydrofuran-2-ylmethyl)methanamine;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]methyl}aniline;
6-({2-Chloro-5-[1-{3-[(3S)-3-methylmorpholin-4-yl]propyl]-5-(methylsulfonyl)-4,5,6,7-tetrahydro-1H-pyrazolo[4,3-c]pyridin-3-yl]phenyl}ethynyl)-2-cyclopropyl-1,2,3,4-tetrahydroisoquinoline;
3-(4-Chloro-3-{[3-({[(2-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-chlorophenyl)methyl]amino(methyl)phenyl]ethynyl}-phenyl)-1-(3-thiomorpholin-4-ylpropyl)-1,4,6,7-tetrahydro-5H-pyrazolo[4,3-c]pyridine-5-carboxamide;
3-(4-Chloro-3-[3-({[(2-fluorophenyl)methyl]amino]methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[3-({[(3-fluorophenyl)methyl]amino-methyl)phenyl]ethynyl(phenyl)-1-(3-thiomorpholin-4-ylpropyl)-1,4,6,7-tetrahydro-5H-pyrazolo[4,3-c]pyridine-5-carboxamide;
3-(4-Chloro-3-[3-({[(4-fluorophenyl)methyl]amino]methyl)phenyl]ethynyl]phenyl)-1-(3-thiomorpholin-4-ylpropyl)-1,4,6,7-tetrahydro-5H-pyrazolo[4,3-c]pyridine-5-carboxamide;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-methyl-1,2,3,4-tetrahydroisoquinoline;
6-({2-Chloro-5-[1-{3-[(3S)-3-methylmorpholin-4-yl]propyl]-5-(methylsulfonyl)-4,5,6,7-tetrahydro-1H-pyrazolo[4,3-c]pyridin-3-yl]phenyl}ethynyl)-2-methyl-1,2,3,4-tetrahydroisoquinoline;
N-{[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenylaethynyl)phenyl]methyl}-2-methylpropan-1-amine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-3-ylmethyl)methanamine;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(pyridin-4-ylmethyl)methanamine;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(2-methylpropyl)-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-{[4-(methylsulfonyl)phenyl]methyl}methanamine;
1,1-Dimethylethyl 6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-1-oxo-3,4-dihydroisoquinoline-2(1H)-carboxylate;
Ethyl [6-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-3,4-dihydroisoquinolin-2(1H)-yl]acetate;
6-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-prop-2-en-1-yl-1,2,3,4-tetrahydroisoquinoline;
1-[3-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-methyl-N-(phenylmethyl)methanamine;
(2R)-1-[3-(4-Chloro-3-{[4-({[(4-chlorophenyl)methyl]amino]methyl)phenyl]ethynyl]phenyl)-5-(methylsulfonyl)-4,5,6,7-tetrahydro-1H-pyrazolo[4,3-c]pyridin-1-yl]-3-[(3S)-3-methylmorpholin-4-yl]propan-2-ol;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-methyl-N-(phenylmethyl)methanamine;
N-{[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenylethyl)phenyl]methyl}-2-methylpropan-1-amine;
3-(4-Chloro-3-{[4-([(2-methylphenyl)methyl]aminomethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-{(4-[({[2-(methyloxy)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-{(4-[({[3-(methyloxy)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
1-[3-(2-{2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethyl)phenyl]-N-(phenylmethyl)methanamine;
1-[4-({2-Chloro-5-[1-{3-[(3R)-3-methylmorpholin-4-yl]propyl}-5-(methylsulfonyl)-4,5,6,7-tetrahydro-1H-pyrazolo[4,3-c]pyridin-3-yl]phenyl}ethynyl)phenyl]-N-[(4-chlorophenyl)methyl]methanamine;
1-{4-[(2-Chloro-5-{5-(methylsulfonyl)-1-[3-(1-oxidothiomorpholin-4-yl)propyl]-4,5,6,7-tetrahydro-1H-pyrazolo[4,3-c]pyridin-3-yl}phenyl)ethynyl]phenyl}-N-[(4-chlorophenyl)methyl]methanamine;
3-[4-Chloro-3-(−4-[({[4-(methyloxy)phenyl]methyl]amino)methyl]phenyl(ethynyl)phenyl]-1-(3-thiomorpholin-4-ylpropyl)-1,4,6,7-tetrahydro-5H-pyrazolo[4,3-c]pyridine-5-carboxamide;
1-[5-({2-Chloro-5-[1-{3-[(3S)-3-methylmorpholin-4-yl]propyl}-5-(methylsulfonyl)-4,5,6,7-tetrahydro-1H-pyrazolo[4,3-c]pyridin-3-yl]phenyl}ethynyl)-2-fluorophenyl]-N-(phenylmethyl)methanamine;
1-[5-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)-2-(methyloxy)phenyl]-N-(phenylmethyl)methanamine;
(3R)-7-({2-Chloro-5-[5-(methylsulfonyl)-1-(3-morpholin-4-ylpropyl)-4,5,6,7-tetrahydro-1H-pyrazolo[4,3-c]pyridin-3-yl]phenyl-4-ethynyl)-3-(morpholin-4-ylcarbonyl)-1,2,3,4-tetrahydroisoquinoline;
3-(4-Chloro-3-{[4-({[(2-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(3-fluorophenyl)methyl]amino-methyl)phenyl]ethynyl(phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-({[(4-fluorophenyl)methyl]amino-methyl)phenyl]ethynyl]phenyl)-1-(3-thiomorpholin-4-ylpropyl)-1,4,6,7-tetrahydro-5H-pyrazolo[4,3-c]pyridine-5-carboxamide;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-(phenylmethyl)methanamine;
1-[2-Chloro-4-({2-chloro-5-[5-(methylsulfonyl)-1-(3-morpholin-4-ylpropyl)-4,5,6,7-tetrahydro-1H-pyrazolo[4,3-c]pyridin-3-yl]phenyl}ethynyl)phenyl]-N-[(4-chlorophenyl)methyl]methanamine;
3-(4-Chloro-3-{[4-([(4-chlorophenyl)methyl]aminocarbonyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(morpholin-2-ylmethyl)oxy]methyl]phenyl)ethynyl]phenyl}-1-(3-thiomorpholin-4-ylpropyl)-1,4,6,7-tetrahydro-5H-pyrazolo[4,3-c]pyridine-5-carboxamide;
3-(4-Chloro-3-{[4-({4-[(4R)-4-hydroxy-2-oxopyrrolidin-1-yl]piperidin-1-yl}methyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-[4-Chloro-3-({4-[({[4-(methylsulfanyl)phenyl]methyl]amino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-(3-{[4-({[(4-Aminophenyl)methyl]amino]methyl)phenyl]ethynyl}-4-chlorophenyl)-1-(3-thiomorpholin-4-ylpropyl)-1,4,6,7-tetrahydro-5H-pyrazolo[4,3-c]pyridine-5-carboxamide;
3-{4-Chloro-3-[(4-{[(3R)-3-hydroxypyrrolidin-1-yl]carbonyl}phenyl)ethynyl]phenyl}-1-(3-thiomorpholin-4-ylpropyl)-1,4,6,7-tetrahydro-5H-pyrazolo[4,3-c]pyridine-5-carboxamide;
N-[(2-Chloro-5-{[2-chloro-5-(1-{3-[(3S)-3-methylmorpholin-4-yl]propyl}-4,5,6,7-tetrahydro-1H-pyrazolo[4,3-c]pyridin-3-yl)phenyl]ethynyl}phenyl)methyl]glycine;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(3-hydroxypropyl)benzamide;
3-[4-Chloro-3-({4-chloro-3-[(3-hydroxypropyl)carbamoyl]phenyl(ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(2S)-pyrrolidin-2-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(2S)-pyrrolidin-2-ylmethyl]benzamide;
3-{4-Chloro-3-[(4-chloro-3-{[(2S)-pyrrolidin-2-yl methyl]carbamoyl}phenyl)ethynyl]phenyl}-[1-(3-thiomorpholin-4-ylpropyl)-1,4,6,7-tetrahydro-5H-pyrazolo[4,3-c]pyridine-5-carboxamide;
tert-Butyl 3-[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorobenzyl)oxy]pyrrolidine-1-carboxylate;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(3-hydroxypropyl)benzamide;
2-(3-{4-Chloro-3-[(4-chloro-3-{[(2-hydroxy-2-methylpropyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-[3-{4-Chloro-3-[(4-chloro-3-{[(2-hydroxy-2-methylpropyl)amino]methyl}phenyl)ethynyl]phenyl}-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-({[(1R)-2-hydroxy-1-phenylethyl]amino}methyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(1R)-2-hydroxy-1-phenylethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[3-(methylamino)propyl]benzamide;
2-(3-{4-Chloro-3-({4-chloro-3-[(ethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
2-[3-{[4-Chloro-3-{[(4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridin-5-yl]-2-oxoacetamide;
3-(4-Chloro-3-{[4-chloro-3-(pyrrolidin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(piperazin-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(1,4-diazepan-1-ylcarbonyl)phenyl]ethynyl}phenyl)-1-(3-thiomorpholin-4-ylpropyl)-1,4,6,7-tetrahydro-5H-pyrazolo[4,3-c]pyridine-5-carboxamide;
2-[3-{[4-Chloro-3-{[(4-chloro-3-(pyrrolidin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(piperazin-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-[3-{[4-Chloro-3-{[(4-chloro-3-(1,4-diazepan-1-ylcarbonyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)methylcarbamate;
tert-Butyl (3-{[(5-{[5-(5-[amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chlorophenyl)carbonyl]amino}propyl)carbamate;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-[(3R)-pyrrolidin-3-ylmethyl]benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(piperidin-3-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(morpholin-2-ylmethyl)benzamide;
5-[(5-{5-[Amino(oxo)acetyl]-1-(3-thiomorpholin-4-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
3-{4-Chloro-3-[(4-chloro-3-[(3R)-pyrrolidin-3-ylmethyl]carbamoyl-phenyl)ethynyl]phenyl}-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(piperidin-3-ylmethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(morpholin-2-ylmethyl)carbamoyl]phenyl(ethynyl)phenyl]-1-(3-thiomorpholin-4-ylpropyl)-1,4,6,7-tetrahydro-5H-pyrazolo[4,3-c]pyridine-5-carboxamide;
3-[4-Chloro-3-({4-chloro-3-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-[(3R)-pyrrolidin-3-ylmethyl]benzamide;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-2-chloro-N-(piperidin-3-ylmethyl)benzamide;
2-[3-{[4-Chloro-3-{[(4-chloro-3-(morpholin-4-ylmethyl)phenyl]ethynylphenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-({4-chloro-3-[(cyclopropylamino)methyl]phenyl(ethynyl)phenyl]-1-{3-[(3S)-3-methylmorpholin-4-yl]propyl}-1,4,6,7-tetrahydro-5H-pyrazolo[4,3-c]pyridin-5-yl)-2-oxoacetamide;
3-[4-Chloro-3-(4-chloro-3-[(2-morpholin-4-ylethyl)carbamoyl]phenyl}ethynyl)phenyl]-1-(3-piperidin-1-ylpropyl)-1,4,6,7-tetrahydro-5H-pyrazolo[4,3-c]pyridine-5-carboxamide;
5-[(5-{5-[Amino(oxo)acetyl]-1-(3-piperidin-1-ylpropyl)-4,5,6,7-tetrahydro-1H-pyrazolo[4,3-c]pyridin-3-yl}-2-chlorophenyl)ethynyl]-2-chloro-N-(2-morpholin-4-ylethyl)benzamide;
2-{3-[4-Chloro-3-{(4-chloro-3-[(ethylamino) methyl]phenylethynyl)phenyl]-1-(3-piperidin-1-ylpropyl)-1,4,6,7-tetrahydro-5H-pyrazolo[4,3-c]pyridin-5-yl}-2-oxoacetamide;
2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]ethanamine;
2-{3-[4-Chloro-3-({4-chloro-3-[(cyclopentylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
3-[4-Chloro-3-({4-chloro-3-[(cyclopropylamino)methyl]phenyl}ethynyl)phenyl]-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopentanamine;
5-{[5-(5-[Amino(oxo)acetyl]-1-{3-[(3S)-3-methylmorpholin-4-yl]propyl}-4,5,6,7-tetrahydro-1H-pyrazolo[4,3-c]pyridin-3-yl)-2-chlorophenyl]ethynyl}-N-(azetidin-3-ylmethyl)-2-chlorobenzamide;
3-[4-Chloro-3-(4-chloro-3-[(cyclopentylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
2-{3-[4-Chloro-3-(4-chloro-3-[(cyclopropylamino)methyl]phenyl}ethynyl)phenyl]-1-(3-piperidin-1-ylpropyl)-1,4,6,7-tetrahydro-5H-pyrazolo[4,3-c]pyridin-5-yl}-2-oxoacetamide;
2-[3-{[4-Chloro-3-{[(4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridin-5-yl]-2-oxoacetamide;
3-(4-Chloro-3-{[4-chloro-3-(morpholin-4-ylmethyl)phenyl]ethynyl]phenyl)-1-(3-piperidin-1-ylpropyl)-1,4,6,7-tetrahydro-5H-pyrazolo[4,3-c]pyridine-5-carboxamide;
N-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)benzyl]cyclopropanamine;
3-(4-Chloro-3-{[4-chloro-3-(morpholin-4-ylmethyl)phenyl]ethynyl]phenyl)-5-(methylsulfonyl)-1-(3-piperidin-1-ylpropyl)-4,5,6,7-tetrahydro-1H-pyrazolo[4,3-c]pyridine;
3-[4-Chloro-3-(4-chloro-3-[(cyclopropylamino) methy]phenyl]ethynyl)phenyl]-1-(3-thiomorpholin-4-ylpropyl)-1,4,6,7-tetrahydro-5H-pyrazolo[4,3-c]pyridine-5-carboxamide;
3-(4-Chloro-3-{[4-chloro-3-(morpholin-4-ylmethyl)phenyl]ethynyl]phenyl)-1-(3-thiomorpholin-4-ylpropyl)-1,4,6,7-tetrahydro-5H-pyrazolo[4,3-c]pyridine-5-carboxamide;
3-[4-Chloro-3-{(4-[(2-morpholin-4-ylethyl)carbamoyl]phenyl}ethynyl)phenyl]-1-(3-thiomorpholin-4-ylpropyl)-1,4,6,7-tetrahydro-5H-pyrazolo[4,3-c]pyridine-5-carboxamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-(2-morpholin-4-ylethyl)benzamide;
3-{4-Chloro-3-[(4-[(2S)-pyrrolidin-2-ylmethyl]carbamoyl]phenyl)ethynyl]phenyl]-1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1 {3-[(3S)-3-methylmorpholin-4-yl]propyl}-4,5,6,7-tetrahydro-1H-pyrazolo[4,3-c]pyridin-3-yl)-2-chlorophenyl]ethynyl]-N-[(2S)-pyrrolidin-2-ylmethyl]benzamide;
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[(2S)-pyrrolidin-2-ylmethyl]benzamide;
3-{4-Chloro-3-[(4-{[3-(methylamino)propyl]carbamoyl}phenyl)ethynyl]phenyl}-1-(3-thiomorpholin-4-ylpropyl)-1,4,6,7-tetrahydro-5H-pyrazolo[4,3-c]pyridine-5-carboxamide;
4-{[5-(5-[Amino(oxo)acetyl]-1 {3-[(3S)-3-methylmorpholin-4-yl]propyl}-4,5,6,7-tetrahydro-1H-pyrazolo[4,3-c]pyridin-3-yl)-2-chlorophenyl]ethynyl]-N-[3-(methylamino)propyl]benzamide; and
4-({2-Chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)-N-[3-(methylamino)propyl]benzamide;
3-[4-Chloro-3-{(4-chloro-3-[(dimethylamino)methyl]phenyl}ethynyl)phenyl]-1-(3-thiomorpholin-4-ylpropyl)-1,4,6,7-tetrahydro-5H-pyrazolo[4,3-c]pyridine-5-carboxamide;
2-(3-{4-Chloro-3-({4-chloro-3-[(dimethylamino) methyl]phenylethynyl)phenyl]-{3-[(3S)-3-methylmorpholin-4-yl]propyl}-1,4,6,7-tetrahydro-5H-pyrazolo[4,3-c]pyridin-5-yl)-2-oxoacetamide;
1-[2-Chloro-5-({2-chloro-5-[5-(methylsulfonyl)-1-(3-piperidin-1-ylpropyl)-4,5,6,7-tetrahydro-1H-pyrazolo[4,3-c]pyridin-3-yl]phenyl}ethynyl)phenyl]-N,N-dimethylmethanamine;
2-[3-{[4-Chloro-3-{[(4-(hydroxymethyl)phenyl]ethynyl}phenyl)-1-{3-[(3S)-3-methylmorpholin-4-yl]propyl}-1,4,6,7-tetrahydro-5H-pyrazolo[4,3-c]pyridin-5-yl]-2-oxoacetamide;
2-(3-{4-Chloro-3-[(4-chloro-3-{[(1-methylethyl)amino]methyl}phenyl)ethynyl]phenyl}-1-{3-[(3S)-3-methylmorpholin-4-yl]propyl}-1,4,6,7-tetrahydro-5H-pyrazolo[4,3-c]pyridin-5-yl)-2-oxoacetamide;
2-(3-{4-Chloro-3-[(4-{[(4-chlorobenzyl)amino]methyl{phenyl)ethynyl]phenyl}-1-{3-[(3R)-3-methylmorpholin-4-yl]propyl}-1,4,6,7-tetrahydro-5H-pyrazolo[4,3-c]pyridin-5-yl)-2-oxoacetamide;
